(19) United States
(12) Patent Application Publication
(10) Pub. No.: US 2023/0151034 A1
(43) Pub. Date: May 18, 2023

(54) PEPTIDOMIMETIC N5-METHYL-N2-(NONANOYL-L-LEUCYL)-L-GLUTAMINATE DERIVATIVES, TRIAZASPIRO[4.14]NONADECANE DERIVATIVES AND SIMILAR COMPOUNDS AS INHIBITORS OF NOROVIRUS AND CORONAVIRUS REPLICATION

(71) Applicant: COCRYSTAL PHARMA, INC., Bothell, WA (US)

(72) Inventors: Irina C. Jacobson, Sammamish, WA (US); Sam SK Lee, Edmonds, WA (US); Juan Carlos Pizarro Novoa, Bothell, WA (US)

(21) Appl. No.: 17/911,461

(22) PCT Filed: Mar. 17, 2021
(86) PCT No.: PCT/US21/22680

§ 371 Date: Sep. 14, 2022

Related U.S. Application Data
(60) Provisional application No. 63/027,492, filed on May 20, 2020.
(60) Provisional application No. 62/990,721, filed on Mar. 17, 2020.

(51) Int. Cl. C07F 9/40; C07C 271/20; C07D 267/14; C07F 9/6561; C07D 273/00

(52) U.S. Cl. CPC C07F 9/4018; C07C 271/20; C07D 267/14; C07F 9/6561; C07D 273/00

(57) ABSTRACT

Peptidomimetic N5-methyl-N2-(nonanoyl-L-leucyl)-L-glutaminate derivatives, triazaspiro[4.14]nonadecane derivatives and similar compounds for use in methods of inhibiting the replication of noroviruses and coronaviruses in a biological sample or patient, for use in reducing the amount of noroviruses or coronaviruses in a biological sample or patient, and for use in treating norovirus and coronavirus in a patient, comprising administering to said biological sample or patient a safe and effective amount of a compound represented by formulae I or II, or a pharmaceutically acceptable salt thereof. The present description discloses the synthesis and characterisation of exemplary compounds as well as pharmacological data thereof (e.g. page 99 to page 271; examples 1 to 3; compounds A1 to A104 and B1 to B66; tables A to E).

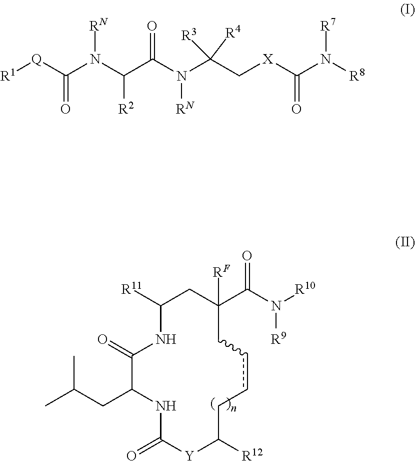

### FIELD OF THE DISCLOSURE

This disclosure relates generally to inhibitors of norovirus and coronavirus replication, and methods of treating or preventing norovirus and coronavirus infections by administering the inhibitors to a patient in need of treatment thereof.

### BACKGROUND

Noroviruses are important enteric pathogens involved in non-bacterial gastroenteritis outbreaks worldwide. Noroviruses mainly occur from person to person via the fecal-oral route but also through contaminated food or water. Indirect contamination is also possible owing to the persistence of the virus in the environment. Human noroviruses belong to the genus Norovirus, family Caliciviridae and are non-enveloped viruses with a positive-sense, single-stranded RNA genome. Norovirus strains are classified into seven groups. Viruses belonging to groups GI, GII, and GIV infect humans, while groups GII, GIII, GIV, GV, GVI and GVII NoVs have been described in animals.

Coronaviruses are a family of common viruses that cause a range of illnesses in humans from the common cold to severe acute respiratory syndrome (SARS). Coronaviruses can also cause a number of diseases in animals. Coronaviruses are enveloped, positive-stranded RNA viruses whose name derives from their characteristic crown-like appearance in electron micrographs. Coronaviruses are classified as a family within the Nidovirales order, viruses that replicate using a nested set of mRNAs. The coronavirus subfamily is further classified into four genera: alpha, beta, gamma, and delta coronaviruses. The human coronaviruses (HCoVs) are in two of these genera: alpha coronaviruses (including HCoV-229E and HCoV-NL63) and beta coronaviruses (including HCoV-HKU1, HCoV-OC43, Middle East respiratory syndrome coronavirus (MERS-CoV), the severe acute respiratory syndrome coronavirus (SARS-CoV), and SARS-CoV-2).

In 2012, a novel coronavirus emerged in Saudi Arabia and became known as Middle East Respiratory Syndrome coronavirus (MERS-CoV). About half of reported cases of MERS-CoV infection have resulted in death and a majority of reported cases have occurred in older to middle age men. Only a small number of reported cases involved subjects with mild respiratory illness. Human to human transmission of MERS-CoV has been found to be possible, but very limited. Another novel coronavirus emerged in Wuhan, China in late 2019. This virus is known as SARS-CoV-2, 2019-nCoV, or Wuhan coronavirus, and it the cause of a worldwide pandemic in late 2019 and 2020.

Given the widespread transmission and potential health effects of these viruses, there is a need for drugs for treating norovirus and coronavirus infections.

### SUMMARY

The present disclosure generally relates to methods of treating norovirus and coronavirus infections, to methods of inhibiting the replication of noroviruses and coronaviruses, to methods of reducing the amount of noroviruses and coronaviruses, and to compounds and compositions that can be employed for such methods.

The disclosure provides compounds of Formula (I), and pharmaceutically acceptable salts thereof:

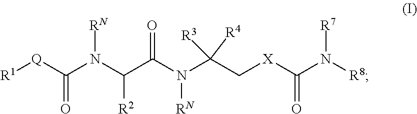

wherein X is NR^{N}, O, or CR⁵R⁶; Q is O, NR^{O}, or a bond; each R^{N} is independently H or C₁₋₆alkyl; each R^{O} is independently H or C₁₋₆alkyl; R¹ is C₁₋₈alkyl, C₁₋₁₂alkylene-C₆₋₁₀aryl, 5- to 12-membered heterocycle having 1 to 3 ring heteroatoms selected from N, O, and S, or C₅₋₈carbocyclyl, and the C₁₋₁₂alkylene is optionally substituted with a C₃₋₅ carbocycle, the C₆₋₁₀aryl is optionally substituted with 1-3 halo, the 5- to 12-membered heterocycle is optionally substituted with 1-3 substituents independently selected from COO—C₁₋₆alkyl, C₁₋₆ alkylene-C₆₋₁₀aryl, and SO₂—C₁₋₆alkyl, and the C₅₋₈carbocyclyl is optionally substituted with C₆₋₁₀aryl or C₆₋₁₀aryl substituted with 1-3 halo; or R^{O} and R¹ together with the nitrogen to which they are attached form a 5- to 12-membered heterocycle having 1-3 ring heteroatoms selected from N, O, and S, and optionally substituted with 1-3 halo; R² is C₁₋₆ alkyl or C₁₋₆alkylene-C₅₋₈carbocyclyl, wherein C₅₋₈carbocyclyl is optionally substituted with 1-3 substituents independently selected from C₁₋₆alkyl and halo; R³ is H or C₁₋₆alkyl; R⁴ is C₁₋₆alkylene-OH, C₁₋₆alkylene-OH substituted with PO(OCH₂CH₂)₂ or SO₃H, CHO, C(O)-(4-8 membered heterocycle having 1-3 ring heteroatoms selected from N, O, and S, CONR^{A}R^{B}, or C(O)—C(O)NR^{N}—Y¹—X¹-A, wherein A is C₅₋₈carbocyclyl, 4-12-membered heterocycle having 1-3 ring heteroatoms selected from N, O, and S, C₆₋₁₀aryl, or 5-8-membered heteroaryl having 1-3 ring heteroatoms selected from N, O, and S, and the carbocyclyl, heterocyclyl, aryl, or heteroaryl is optionally substituted with 1-2 substituents independently selected from halo, C₁₋₆alkyl, and COO—C₁₋₆alkyl; Y¹ is C₁₋₆alkylene optionally substituted with 1-3 substituents independently selected from halo, OH, NR^{N}R^{N}, and C₁₋₆alkoxy; X¹ is null, NR^{N}R^{N}, C(O), SO₂, or OC(O); R⁵ and R⁶ are each independently H or C₁₋₆alkyl; R⁷ is C₁₋₆alkyl, C₁₋₆alkylene-O—C₁₋₆alkyl, C₁₋₆alkylene-O—C₁₋₆alkylene-O—C₁₋₆ alkyl, C₁₋₆alkylene-CONH—C₁₋₆alkyl, C₁₋₆alkylene-CON(CH₃)₂, C₁₋₆alkylene-NHCONH—C₁₋₆alkyl, C₁₋₆alkylene-OCONH—C₁₋₆ alkyl, C₁₋₆alkylene-NHCOO—C₁₋₆alkyl, C₁₋₆alkylene-NHSO₂—C₁₋₆alkyl, C₁₋₆alkylene-C₆₋₁₀aryl and the C₁₋₆alkylene is optionally substituted with C₃₋₅carbocyclyl, or C₁₋₆alkylene-5-8 membered heteroaryl having 1-3 ring heteroatoms selected from N, O, and S, wherein C₆₋₁₀aryl and 5-8 membered heteroaryl are optionally substituted with 1-3 substituents independently selected from C₁₋₆alkoxy and halo; R⁸ is H or C₁₋₆alkyl; or R⁴ and R⁸ together with the atoms to which they are attached form a 5- to 8-membered heterocycle having 1-3 ring heteroatoms selected from N, O, and S, optionally substituted with 1-3 R^{C}; or R⁶ and R⁸ together with the atoms to which they are attached form a 5- to 8-membered heterocycle having 1-3 ring heteroatoms selected from N, O, and S, optionally substituted with CN, C₁₋₆ alkylene-O—C₁₋₆alkyl, C₁₋₆alkylene-O—C₁₋₆alkylene-O—C₁₋₆alkyl, C₁₋₆alkylene-C₆₋₁₀aryl, or a 6-membered heterocycle having 1-3 ring heteroatoms selected from N, O, and S, and R⁷ can be H; or R⁷ and R⁸ together with the nitrogen to which they are attached form a 5-12 membered heterocycle having 1-3 ring heteroatoms selected from N, O, and S, optionally substituted with halo, C₆₋₁₀aryl, or COO—C₁₋₆alkyl; R^{A} and R^{B} are each independently H, C₁₋₆alkyl, or C₁₋₆alkoxy; and each R^{C} is independently OH or CONH(C₁₋₆alkyl).

In some cases, X is NH, O, or CR⁵R⁶; Q is O, NR^{N}, or a bond; each R^{N} is independently H or C₁₋₆alkyl; R¹ is C₁₋₈alkyl, C₁₋₁₂alkylene-C₆₋₁₀aryl, 5- to 12-membered heterocycle, or C₅₋₈carbocyclyl, wherein C₁₋₁₂alkylene is optionally substituted with a C₃₋₅ carbocycle, C₆₋₁₀aryl is optionally substituted with 1-3 halo, 5- to 12-membered heterocycle is optionally substituted with COO—C₁₋₆alkyl or SO₂—C₁₋₆alkyl, and C₅₋₈carbocyclyl is optionally substituted with C₆₋₁₀aryl; R² is C₁₋₆alkyl or C₁₋₆alkylene-C₅₋₈carbocyclyl, wherein C₅₋₈carbocyclyl is optionally substituted with 1-3 substituents independently selected from C₁₋₆alkyl and halo; R³ is H or C₁₋₆alkyl; R⁴ is C₁₋₆alkylene-OH, C₁₋₆alkylene-OH substituted with PO(OCH₂CH₂)₂, C₁₋₆alkylene-OH substituted with SO₃H, CHO, or CONR^{A}R^{B}; R⁵ and R⁶ are each independently H or C₁₋₆alkyl; R⁷ is C₁₋₆alkyl, C₁₋₆alkylene-O—C₁₋₆alkyl, C₁₋₆alkylene-O—C₁₋₆alkylene-O—C₁₋₆alkyl, C₁₋₆alkylene-CONH—C₁₋₆ alkyl, C₀₋₆alkylene-CON(CH₃)₂, C₁₋₆alkylene-NHCONH—C₁₋₆alkyl, C₁₋₆alkylene-OCONH—C₁₋₆alkyl, C₁₋₆alkylene-NHCOO—C₁₋₆alkyl, C₁₋₆alkylene-NHSO₂—C₁₋₆alkyl, C₁₋₆alkylene-C₆₋₁₀aryl wherein C₁₋₆alkylene is optionally substituted with C₃₋₅ carbocyclyl, or C₁₋₆alkylene-5-8 membered heteroaryl, wherein C₆₋₁₀aryl and 5-8 membered heteroaryl are optionally substituted with 1-3 substituents selected from C₁₋₆alkoxy and halo; R⁸ is H or C₁₋₆alkyl; or R⁴ and R⁸ together with the atoms to which they are attached form a 6-membered heterocycle optionally substituted with 1-3 R^{C}; or R⁶ and R⁸ together with the atoms to which they are attached form a 5-membered heterocycle optionally substituted with CN, C₁₋₆ alkylene-O—C₁₋₆alkyl, C₁₋₆alkylene-O—C₁₋₆alkylene-O—C₁₋₆alkyl, C₁₋₆alkylene-C₆₋₁₀aryl, or a 6-membered heterocycle, and R⁷ can be H; or R⁷ and R⁸ together with the nitrogen to which they are attached form a 5-12 membered heterocycle optionally substituted with halo, C₆₋₁₀aryl, or COO—C₁₋₆alkyl; R^{A} and R^{B} are each independently H, C₁₋₆alkyl, or C₁₋₆alkoxy; and each R^{C} is independently OH or CONH(C₁₋₆alkyl).

In some cases, the compounds of Formula (I) have the structure of Formula (IA):

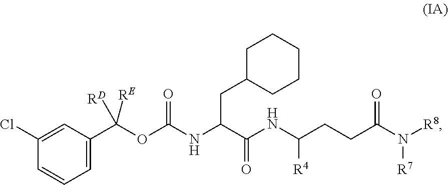

wherein R^{D} is H and R^{E} is H or C₁₋₆ alkyl or R^{D} and R^{E}together with the carbon to which they are attached form a 3-5 membered carbocycle.

Also provided herein are compounds of Formula (II), and pharmaceutically acceptable salts thereof:

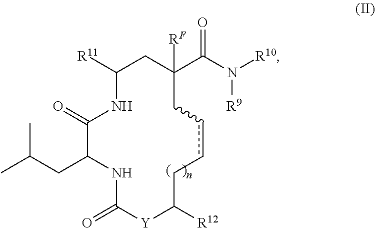

wherein Y is O or a bond; R^{F} is H, or R^{F} and R¹⁰ together with the atoms to which they are attached form a five-membered heterocycle; R⁹ is C₁₋₆alkyl, or C₁₋₆alkylene-C₆₋₁₀aryl, wherein C₆₋₁₀aryl is optionally substituted with 1-3 substituents independently selected from C₁₋₃alkoxy and halo; R¹⁰ is H or C₁₋₆alkyl; R¹¹ is C₁₋₆alkylene-OH, C₁₋₆alkylene-OH substituted with PO(OCH₂CH₂)₂, CHO, or (CO)₁₋₂NR¹³R¹⁴; or R¹⁰ and R¹¹ together with the atoms to which they are attached form a six-membered heterocycle optionally substituted with 1-3 substituents independently selected from OH and CONR¹³R¹⁴; R¹² is H, C₁₋₈alkyl, or C₁₋₆alkylene-C₆₋₁₀aryl, wherein C₆₋₁₀aryl is optionally substituted with 1-3 substituents independently selected from C₁₋₃alkoxy and halo; R¹³ and R¹⁴ are each independently H, C₁₋₆alkyl, or C₁₋₆alkoxy; [symbol] indicates a single or a double bond; and n is 1-3.

In some cases, the compound of Formula (II) has a structure of Formula (IIA), (IIB), (IIC), or (IID):

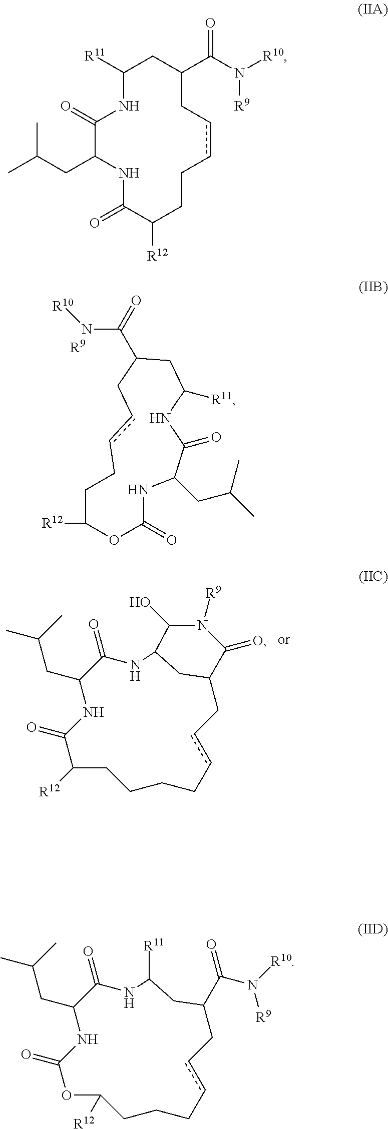

Further provided are methods of administering to a biological sample or patient a safe and effective amount of a compound as disclosed herein, e.g., as represented by Formulas I, IA, II, IIA, IIB, IIC, or IID.

Also provided herein are methods of reducing the amount of virus in a biological sample or in a patient by administering to said biological sample or patient an effective amount of a compound as disclosed herein, e.g., as represented by any of Formulas I, IA, II, IIA, IIB, IIC, or IID or a compound of Table A or B.

Further provided are methods of treating or preventing a viral infection in a patient, comprising administering to said patient an effective amount of a compound as disclosed herein, e.g., as represented by Formulas I, IA, II, IIA, IIB, IIC, or IID or a compound of Table A or B.

Also provided are pharmaceutical compositions comprising a compound as disclosed herein, e.g., as represented by any of Formulas I, IA, II, IIA, IIB, IIC, or IID, or a compound of Table A or B, or a pharmaceutically acceptable salt thereof, and a pharmaceutically acceptable excipient, carrier, adjuvant or vehicle.

Also provided are uses of a compound described herein for inhibiting or reducing the replication of virus in a biological sample or patient, for reducing the amount of virus in a biological sample or patient, or for treating a viral infection in a patient.

Further provided herein are uses of a compound described herein for the manufacture of a medicament for treating a viral infection in a patient, for reducing the amount of virus in a biological sample or in a patient, or for inhibiting the replication of virus in a biological sample or patient.

### DETAILED DESCRIPTION

Provided herein are compounds, and their use in treating or preventing a viral infection (e.g., a norovirus or coronavirus infection). Also provided are uses of the compounds described herein, or pharmaceutically acceptable salts thereof, or pharmaceutically acceptable compositions comprising such a compound or a pharmaceutically acceptable salt thereof, for inhibiting the replication of viruses in a biological sample or in a patient, for reducing the amount of viruses (reducing viral titer) in a biological sample or in a patient, and for treating a viral infection in a patient.

Unless otherwise indicated, structures depicted herein are also meant to include all isomeric (e.g., enantiomeric, diastereomeric, cis-trans, conformational, and rotational) forms of the structure. For example, the R and S configurations for each asymmetric center, (Z) and (E) double bond isomers, and (Z) and (E) conformational isomers are included in this disclosure, unless only one of the isomers is specifically indicated. Therefore, single stereochemical isomers as well as enantiomeric, diastereomeric, cis/trans, conformational, and rotational mixtures of the present compounds are within the scope of the disclosure. In some cases, the compounds disclosed herein are stereoisomers. “Stereoisomers” refer to compounds that differ in the chirality of one or more stereocenters. Stereoisomers include enantiomers and diastereomers. The compounds disclosed herein can exist as a single stereoisomer, or as a mixture of stereoisomers. Stereochemistry of the compounds shown herein indicate a relative stereochemistry, not absolute, unless discussed otherwise. As indicated herein, a single stereoisomer, diastereomer, or enantiomer refers to a compound that is at least more than 50% of the indicated stereoisomer, diastereomer, or enantiomer, and in some cases, at least 90% or 95% of the indicated stereoisomer, diastereomer, or enantiomer.

Unless otherwise indicated, all tautomeric forms of the compounds of the disclosure are within the scope of the disclosure.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this disclosure. Such compounds are useful, for example, as analytical tools or probes in biological assays. Such compounds, especially deuterium analogs, can also be therapeutically useful.

The compounds of the disclosure are defined herein by their chemical structures and/or chemical names. Where a compound is referred to by both a chemical structure and a chemical name, and the chemical structure and chemical name conflict, the chemical structure is determinative of the compound's identity.

### I. Compounds

Provided herein are compounds of Formula (I), and pharmaceutically acceptable salts thereof:

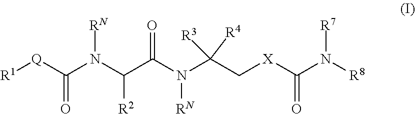

wherein X is NR^{N}, O, or CR⁵R⁶;

Q is O, NR^{O}, or a bond;

each R^{N} is independently H or C₁₋₆alkyl;

each R^{O} is independently H or C₁₋₆alkyl;

R¹ is C₁₋₈alkyl, C₁₋₁₂alkylene-C₆₋₁₀aryl, 5- to 12-membered heterocycle having 1 to 3 ring heteroatoms selected from N, O, and S, or C₅₋₈carbocyclyl, and
the C₁₋₁₂alkylene is optionally substituted with a C₃₋₅ carbocycle,
the C₆₋₁₀aryl is optionally substituted with 1-3 halo,
the 5- to 12-membered heterocycle is optionally substituted with 1-3 substituents independently selected from COO—C₁₋₆alkyl, C₁₋₆alkylene-C₆₋₁₀aryl, and SO₂—C₁₋₆alkyl, and
the C₅₋₈carbocyclyl is optionally substituted with C₆₋₁₀aryl or C₆₋₁₀aryl substituted with 1-3 halo; or

R^{O} and R¹ together with the nitrogen to which they are attached form a 5- to 12-membered heterocycle having 1-3 ring heteroatoms selected from N, O, and S, and optionally substituted with 1-3 halo;

R² is C₁₋₆alkyl or C₁₋₆alkylene-C₅₋₈carbocyclyl, wherein C₅₋₈carbocyclyl is optionally substituted with 1-3 substituents independently selected from C₁₋₆alkyl and halo;

R³ is H or C₁₋₆alkyl;

R⁴ is C₁₋₆alkylene-OH, C₁₋₆alkylene-OH substituted with PO(OCH₂CH₂)₂ or SO₃H, CHO, C(O)-(4-8 membered heterocycle having 1-3 ring heteroatoms selected from N, O, and S, CONR^{A}R^{B}, or C(O)—C(O)NR^{N}—Y¹—X¹-A, wherein A is C₅₋₈carbocyclyl, 4-12-membered heterocycle having 1-3 ring heteroatoms selected from N, O, and S, C₆₋₁₀aryl, or 5-8-membered heteroaryl having 1-3 ring heteroatoms selected from N, O, and S, and the carbocyclyl, heterocyclyl, aryl, or heteroaryl is optionally substituted with 1-2 substituents independently selected from halo, C₁₋₆alkyl, and COO—C₁₋₆alkyl;

Y¹ is C₁₋₆alkylene optionally substituted with 1-3 substituents independently selected from halo, OH, NR^{N}R^{N}, and C₁₋₆alkoxy;

X¹ is null, NR^{N}R^{N}, C(O), SO₂, or OC(O);

R⁵ and R⁶ are each independently H or C₁₋₆alkyl;

R⁷ is C₁₋₆alkyl, C₁₋₆alkylene-O—C₁₋₆alkyl, C₁₋₆alkylene-O—C₁₋₆alkylene-O—C₁₋₆alkyl, C₁₋₆alkylene-CONH—C₁₋₆alkyl, C₀₋₆alkylene-CON(CH₃)₂, C₁₋₆alkylene-NHCONH—C₁₋₆alkyl, C₁₋₆alkylene-OCONH—C₁₋₆alkyl, C₁₋₆alkylene-NHCOO—C₁₋₆alkyl, C₁₋₆alkylene-NHSO₂—C₁₋₆alkyl, C₀₋₆alkylene-C₆₋₁₀aryl and the C₀₋₆alkylene is optionally substituted with C₃₋₅carbocyclyl, or C₁₋₆alkylene-5-8 membered heteroaryl having 1-3 ring heteroatoms selected from N, O, and S, wherein C₆₋₁₀aryl and 5-8 membered heteroaryl are optionally substituted with 1-3 substituents independently selected from C₁₋₆alkoxy and halo;

R⁸ is H or C₁₋₆alkyl; or

R⁴ and R⁸ together with the atoms to which they are attached form a 5- to 8-membered heterocycle having 1-3 ring heteroatoms selected from N, O, and S, optionally substituted with 1-3 R^{C}; or

R⁶ and R⁸ together with the atoms to which they are attached form a 5- to 8-membered heterocycle having 1-3 ring heteroatoms selected from N, O, and S, optionally substituted with CN, C₁₋₆alkylene-O—C₁₋₆alkyl, C₁₋₆alkylene-O—C₁₋₆ alkylene-O—C₁₋₆alkyl, C₀₋₆alkylene-C₆₋₁₀aryl, or a 6-membered heterocycle having 1-3 ring heteroatoms selected from N, O, and S, and R⁷ can be H; or

R⁷ and R⁸ together with the nitrogen to which they are attached form a 5-12 membered heterocycle having 1-3 ring heteroatoms selected from N, O, and S, optionally substituted with halo, C₆₋₁₀aryl, or COO—C₁₋₆alkyl;

R^{A} and R^{B} are each independently H, C₁₋₆alkyl, or C₁₋₆alkoxy; and

each R^{C} is independently OH or CONH(C₁₋₆alkyl).

In some cases, X is NH, O, or CR⁵R⁶;

Q is O, NR^{N}, or a bond;

each R^{N} is independently H or C₁₋₆alkyl;

R¹ is C₁₋₈alkyl, C₁₋₁₂alkylene-C₆₋₁₀aryl, 5- to 12-membered heterocycle, or C₅₋₈carbocyclyl, wherein C₁₋₁₂alkylene is optionally substituted with a C₃₋₅ carbocycle, C₆₋₁₀aryl is optionally substituted with 1-3 halo, 5- to 12-membered heterocycle is optionally substituted with COO—C₁₋₆alkyl or SO₂—C₁₋₆alkyl, and C₅₋₈carbocyclyl is optionally substituted with C₆₋₁₀aryl;

R² is C₁₋₆alkyl or C₁₋₆alkylene-C₅₋₈carbocyclyl, wherein C₅₋₈carbocyclyl is optionally substituted with 1-3 substituents independently selected from C₁₋₆alkyl and halo;

R³ is H or C₁₋₆alkyl;

R⁴ is C₁₋₆alkylene-OH, C₁₋₆alkylene-OH substituted with PO(OCH₂CH₂)₂, C₁₋₆alkylene-OH substituted with SO₃H, CHO, or CONR^{A}R^{B};

R⁵ and R⁶ are each independently H or C₁₋₆alkyl;

R⁷ is C₁₋₆alkyl, C₁₋₆alkylene-O—C₁₋₆alkyl, C₁₋₆alkylene-O—C₁₋₆alkylene-O—C₁₋₆alkyl, C₁₋₆alkylene-CONH—C₁₋₆alkyl, C₁₋₆alkylene-CON(CH₃)₂, C₁₋₆alkylene-NHCONH—C₁₋₆alkyl, C₁₋₆alkylene-OCONH—C₁₋₆alkyl, C₁₋₆alkylene-NHCOO—C₁₋₆alkyl, C₁₋₆alkylene-NHSO₂—C₁₋₆alkyl, C₁₋₆alkylene-C₆₋₁₀aryl wherein C₁₋₆alkylene is optionally substituted with C₃₋₅carbocyclyl, or C₁₋₆alkylene-5-8 membered heteroaryl, wherein C₆₋₁₀aryl and 5-8 membered heteroaryl are optionally substituted with 1-3 substituents selected from C₁₋₆alkoxy and halo;

R⁸ is H or C₁₋₆alkyl; or

R⁴ and R⁸ together with the atoms to which they are attached form a 6-membered heterocycle optionally substituted with 1-3 R^{C}; or

R⁶ and R⁸ together with the atoms to which they are attached form a 5-membered heterocycle optionally substituted with CN, C₁₋₆alkylene-O—C₁₋₆alkyl, C₁₋₆alkylene-O—C₁₋₆alkylene-O—C₁₋₆alkyl, C₁₋₆alkylene-C₆₋₁₀aryl, or a 6-membered heterocycle, and R⁷ can be H; or

R⁷ and R⁸ together with the nitrogen to which they are attached form a 5-12 membered heterocycle optionally substituted with halo, C₆₋₁₀aryl, or COO—C₁₋₆alkyl;

R^{A} and R^{B} are each independently H, C₁₋₆alkyl, or C₁₋₆alkoxy; and

R^{C} is OH or CONH(C₁₋₆alkyl).

As used herein, the term “alkyl” or “alkylene” as used herein means a saturated straight or branched chain hydrocarbon. Each of “alkyl” and “alkylene” as used herein can be optionally substituted as set forth below. The term Cₙmeans the alkyl group has “n” carbon atoms. For example, C₄alkyl refers to an alkyl group that has 4 carbon atoms. C₁₋₈alkyl refers to an alkyl group having a number of carbon atoms encompassing the entire range (i.e., 1 to 8 carbon atoms), as well as all subgroups (e.g., 1-6, 2-6, 1-5, 2-6, 1-7, 2-7, 1-4, 2-5, 1, 2, 3, 4, 5, 6, 7, and 8 carbon atoms). In some embodiments, the “alkyl” is C₁-C₁₂ alkyl, C₁-C₈ alkyl, or C₁-C₆ alkyl. “C₀ alkylene” refers to a bond. Specific examples include, but are not limited to, methyl, ethyl, isopropyl, n-propyl, sec-butyl, and t-butyl.

As used herein, the term “alkoxy” refers to an alkyl group, as previously defined, attached to the molecule through an oxygen (“alkoxy” e.g., —O-alkyl) atom.

As used herein, the terms “halogen” and “halo” mean F, Cl, Br, or I.

The term “carbocycle” (or “carbocyclyl” or “carbocyclic”) refers to a non-aromatic monocyclic, fused, bridged or spiro ring system whose ring atoms are carbon and which can be saturated or have one or more units of unsaturation. The carbocycle can have three to eight ring carbon atoms. In some embodiments, the number of carbon atoms is 3 to 5. In various embodiments, the number of carbon atoms is 5 to 8. In some embodiments, the number of carbon atoms is 6. “Fused” bicyclic ring systems comprise two rings which share two adjoining ring atoms. Bridged bicyclic group comprise two rings which share three or four adjacent ring atoms. Spiro bicyclic ring systems share one ring atom. Cycloalkyl groups can include cycloalkenyl groups. Specific examples include, but are not limited to, cyclohexyl, cyclopentenyl, cyclopropyl, and cyclobutyl. A carbocycle ring is unsubstituted or substituted as described herein.

The term “heterocycle” as used herein refers to a non-aromatic monocyclic, fused, spiro or bridged ring system which can be saturated or contain one or more units of unsaturation, having three to twelve ring atoms in which one or more (e.g., one to four, one to three, or one, two, three, or four) ring atoms is a heteroatom selected from, N, S, and O. In some embodiments, the heterocycle comprises up to three (e.g., 1 to 3, 1, 2, or 3) ring heteroatoms selected from N, S and O. In some embodiments, the heterocycle comprises 6 to 12 ring members. In some embodiments, the heterocycle comprises 5-6 ring members. In some embodiments, the heterocycle comprises 5 ring members. In some embodiments, the heterocycle comprises 6 ring members. Examples of heterocycles include, but are not limited to, quinuclidinyl, piperidinyl, piperizinyl, pyrrolidinyl, pyrazolidinyl, imidazolidinyl, azepanyl, diazepanyl, triazepanyl, azocanyl, diazocanyl, triazocanyl, oxazolidinyl, isoxazolidinyl, thiazolidinyl, isothiazolidinyl, oxazocanyl, oxazepanyl, thiazepanyl, thiazocanyl, benzimidazolonyl, tetrahydrofuranyl, tetrahydrothiophenyl, morpholino (including, for example, 3-morpholino, 4-morpholino), 2-thiomorpholino, 3-thiomorpholino, 4-thiomorpholino, 1-pyrrolidinyl, 2-pyrrolidinyl, 3-pyrrolidinyl, pyrrolidin-2-one, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl, indolinyl, tetrahydroquinolinyl, tetrahydroisoquinolinyl, benzothiolanyl, benzodithianyl, 3-(1-alkyl)-benzimidazol-2-onyl, and 1,3-dihydro-imidazol-2-onyl. A heterocycle ring is unsubstituted or substituted as described herein.

The term “aryl” refers to aromatic ring groups have only carbon ring atoms (typically six to fourteen or six to ten) and include monocyclic aromatic rings such as phenyl and fused polycyclic aromatic ring systems in which two or more carbocyclic aromatic rings are fused to one another. Examples include 1-naphthyl, 2-naphthyl, 1-anthracyl and 2-anthracyl. Also included within the scope of the term aryl, as it is used herein, is a group in which an aromatic ring is “fused” to one or more non-aromatic rings (carbocyclic or heterocyclic), such as in an indanyl, phthalimidyl, naphthimidyl, phenanthridinyl, or tetrahydronaphthyl, where the radical or point of attachment is on the aromatic ring. An aryl ring is unsubstituted or substituted as described herein.

The terms “heteroaryl” refers to a heterocycle that is aromatic, having five to eight members (e.g., 5 to 6 members), including monocyclic heteroaromatic rings and polycyclic aromatic rings in which a monocyclic aromatic ring is fused to one or more other aromatic ring. Heteroaryl groups have one or more ring (e.g., 1 to 4, 1 to 3, 1, 2, 3, or 4) heteroatoms selected from N, O, and S. Also included within the scope of the term “heteroaryl”, as it is used herein, is a group in which an aromatic ring is “fused” to one or more non-aromatic rings (carbocyclic or heterocyclic), where the radical or point of attachment is on the aromatic ring. Examples of heteroaryl groups include pyridyl, pyrazinyl, pyrimidinyl, pyridazinyl, imidazolyl, pyrrolyl, pyrazolyl, triazolyl, tetrazolyl, oxazolyl, isoxazolyl, oxadiazolyl, thiazolyl, isothiazolyl or thiadiazolyl including, for example, 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 3-pyrazolyl, 4-pyrazolyl, 1-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, 3-pyridazinyl, 2-thiazolyl, 4-thiazolyl, 5-thiazolyl, 2-triazolyl, 5-triazolyl, tetrazolyl, 2-thienyl, 3-thienyl, isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, pyrazinyl, and 1,3,5-triazinyl. A heteroaryl ring is unsubstituted or substituted as described herein.

As described herein, compounds of the disclosure may optionally be substituted with one or more substituents, such as illustrated generally, or as exemplified by particular classes, subclasses, and species of the disclosure. It will be appreciated that the phrase “optionally substituted” is used interchangeably with the phrase “substituted or unsubstituted.” In general, the term “substituted”, whether preceded by the term “optionally” or not, refers to the replacement of one or more hydrogen radicals in a given structure with the radical of a specified substituent. Unless otherwise indicated, an optionally substituted group may have a substituent at each substitutable position of the group. When more than one position in a given structure can be substituted with more than one substituent selected from a specified group, the substituent may be either the same or different at each position. When the term “optionally substituted” follows a list, said term also refers to all of the substitutable groups in the prior list unless otherwise indicated. For example: if R⁷ is C₆₋₁₀aryl or 5-8 membered heteroaryl optionally substituted with 1-3 substituents selected from C₁₋₆alkoxy and halo, then each of C₆₋₁₀aryl and 5-8 membered heteroaryl can optionally be substituted with 1-3 substituents selected from C₁₋₆alkoxy and halo.

In some cases, the compounds of Formula (I) have the structure of Formula (IA):

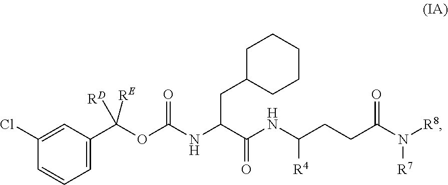

wherein R^{D} is H and R^{E} is H or C₁₋₆ alkyl or R^{D} and R^{E}together with the carbon to which they are attached form a 3-5 membered carbocycle.

In some cases, X is O. In some cases, X is CR⁵R⁶. In some cases, X is CH₂. In some cases, X is NR^{N}. In some cases, X is NH.

In some cases, Q is O. In some cases, Q is NR^{O}. In some cases, Q is NCH₃. In some cases, Q is NH. In some cases, Q is a bond.

In some cases, at least one R^{N} is H. In some cases, at least one R^{N} is C₁₋₆alkyl. In some cases, each R^{N} is H. In some cases, each R^{N} is C₁₋₆alkyl, e.g., methyl.

In some cases, at least one R^{O} is H. In some cases, at least one R^{O} is C₁₋₆alkyl. In some cases, each R^{O} is H. In some cases, each R^{O} is C₁₋₆alkyl, e.g., methyl.

In some cases, R¹ is C₁₋₈alkyl. In some cases, R¹ is C₆alkyl. In some cases, R¹ is C₈alkyl. In some cases, R¹ is C₁₋₆alkylene-C₆₋₁₀aryl. In some cases, C₁₋₆alkylene is substituted with a 3-5 membered carbocycle. In some cases, C₁₋₆alkylene is substituted with a 3 membered carbocycle. In some cases, C₆₋₁₀aryl is unsubstituted. In some cases, C₆₋₁₀aryl is substituted with 1-3 halo. In some cases, R¹ is C₁₋₆alkylene-C₆aryl. In some cases, C₆aryl is substituted with 1-3 halo. In some cases, C₆₋₁₀aryl is substituted with chloro. In some cases, R¹ is chlorobenzyl. In some cases, R¹ is 5-to 12-membered heterocycle having 1 to 3 ring heteroatoms selected from N, O, and S. In some cases, 5- to 12-membered heterocycle is unsubstituted. In some cases, 5- to 12-membered heterocycle is substituted with 1-3 substituents independently selected from COO—C₁₋₆alkyl, C₁₋₆alkylene-C₆₋₁₀aryl, and SO₂—C₁₋₆alkyl. In some cases, 5- to 12-membered heterocycle is substituted with COO—C₁₋₆alkyl or SO₂—C₁₋₆alkyl. In some cases, 5- to 12-membered heterocycle is substituted with COO—C₁₋₆alkyl. In some cases, 5- to 12-membered heterocycle is substituted with SO₂—C₁₋₆alkyl. In some cases, R¹ is 6-membered heterocycle. In some cases, R¹ is C₅₋₈carbocyclyl. In some cases, R¹ is C₆carbocyclyl. In some cases, C₅₋₈carbocyclyl is substituted with C₆₋₁₀aryl. In some cases, C₅₋₈carbocyclyl is substituted with phenyl. In some cases, C₆₋₁₀aryl is substituted with 1-3 halo. In some cases, C₆₋₁₀aryl is substituted with 1-3 chloro. In some cases, R¹ is

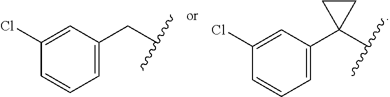 [SMILES: *Cc1cccc(Cl)c1 ; *C1(c2cccc(Cl)c2)CC1]

In some cases, R¹ is

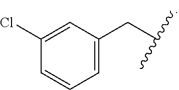 [SMILES: *Cc1cccc(Cl)c1]

In some cases, R¹ is

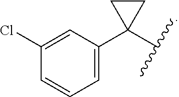 [SMILES: *C1(c2cccc(Cl)c2)CC1]

In some cases, R¹ is

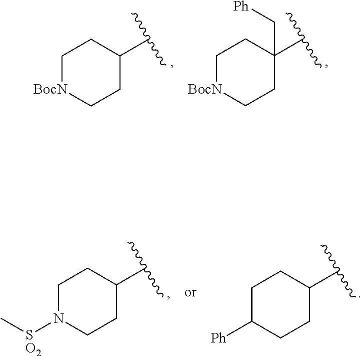 [SMILES: *C1CCN(C(=O)OC(C)(C)C)CC1 ; *C1(Cc2ccccc2)CCN(C(=O)OC(C)(C)C)CC1 ; *C1CCN(S(C)(=O)=O)CC1 ; *C1CCC(c2ccccc2)CC1]

In some cases, R¹ is

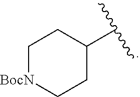 [SMILES: *C1CCN(C(=O)OC(C)(C)C)CC1]

In some cases, R¹ is

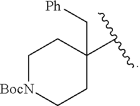 [SMILES: *C1(Cc2ccccc2)CCN(C(=O)OC(C)(C)C)CC1]

In some cases, R¹ is

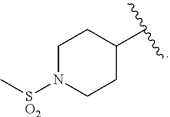 [SMILES: *C1CCN(S(C)(=O)=O)CC1]

In some cases, R¹ is

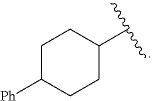 [SMILES: *C1CCC(c2ccccc2)CC1]

In some cases, Q is NR¹. In some embodiments, R^{O} and R¹ together with the nitrogen to which they are attached form a 5- to 12-membered heterocycle optionally substituted with 1-3 halo. In some cases, R^{O} and R¹ together with the nitrogen to which they are attached form a 5- to 8-membered heterocycle optionally substituted with 1-3 halo. In some cases, R^{O} and R¹ together with the nitrogen to which they are attached form a 9-membered heterocycle optionally substituted with 1-3 halo.

In some cases, R¹-Q- is

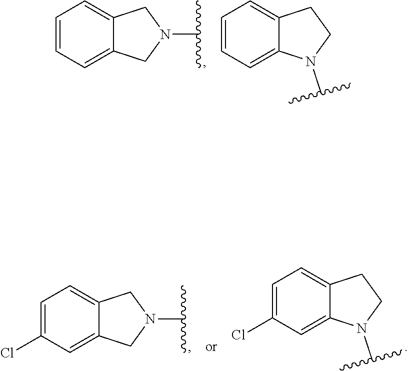

In some cases, R¹-Q- is

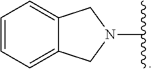 [SMILES: *N1Cc2ccccc2C1]

In some cases, R¹-Q- is

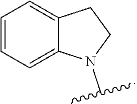 [SMILES: *N1CCc2ccccc21]

In some cases, R¹-Q- is

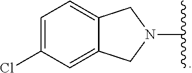 [SMILES: *N1Cc2ccc(Cl)cc2C1]

In some cases, R¹-Q- is

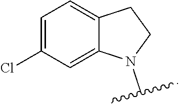 [SMILES: *N1CCc2ccc(Cl)cc21]

In some cases, R² is C₁₋₆alkyl. In some cases, R² is C₄alkyl. In some cases, R² is

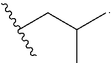 [SMILES: *CC(C)C]

In some cases, R² is C₁₋₆alkylene-C₅₋₈carbocyclyl. In some cases, C₅₋₈carbocyclyl is substituted with 1-3 substituents selected from C₁₋₆alkyl and halo. In some cases, C₅₋₈carbocyclyl is substituted with 1-3 C₁₋₆alkyl. In some cases, C₅₋₈carbocyclyl is substituted with 1-3 halo. In some cases, R² is C₁₋₆alkylene-C₆carbocyclyl. In some cases, R² is

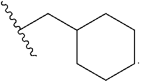 [SMILES: *CC1CCCCC1]

In some cases, R³ is H. In some cases, R³ is C₁₋₆ alkyl. In some cases, R³ is methyl.

In some cases, at least one of R^{D} and R^{E} is H. In some cases, at least one of R^{D} and R^{E} is C₁₋₆ alkyl. In some cases, at least one of R^{D} and R^{E} is C₄alkyl. In some cases, one of R^{D} and R^{E} is

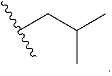 [SMILES: *CC(C)C]

In some cases, R^{D} and R^{E} are each H. In some cases, R^{D} and R^{E} together with the carbon to which they are attached form a 3-5 membered carbocycle. In some cases, R^{D} and R^{E} together with the carbon to which they are attached form a 3 membered carbocycle.

In some cases, R⁴ is C₁₋₆alkylene-OH. In some cases, R⁴ is C₁₋₆alkylene-OH substituted with PO(OCH₂CH₂)₂. In some cases, R⁴ is CHO. In some cases, C(O)-(4-8 membered heterocycle having 1-3 ring heteroatoms selected from N, O, and S. In some cases, R⁴ is CONR^{A}R^{B}. In some cases, at least one of R^{A} and R^{B} is H. In some cases, at least one of R^{A} and R^{B} is C₁₋₆alkyl. In some cases, at least one of R^{A} and R^{B} is C₁₋₆alkoxy. In some cases, R^{A} is C₁₋₆alkyl. In some cases, R^{A} is methyl. In some cases, R^{B} is C₁₋₆alkoxy. In some cases, R^{B} is methoxy. In some cases, R⁴ is

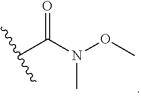 [SMILES: *C(=O)N(C)OC]

In some cases, R⁴ is C₁₋₆alkylene-OH substituted with SO₃H. In some cases, R⁴ is C(O)—C(O)NR^{N}—Y¹—X¹-A, wherein A is C₅₋₈carbocyclyl, 4-12-membered heterocycle having 1-3 ring heteroatoms selected from N, O, and S, C₆₋₁₀aryl, or 5-8-membered heteroaryl having 1-3 ring heteroatoms selected from N, O, and S, and the carbocyclyl, heterocyclyl, aryl, or heteroaryl is optionally substituted with 1-2 substituents independently selected from halo, C₁₋₆alkyl, and COO—C₁₋₆alkyl, Y¹ is C₁₋₆alkylene optionally substituted with 1-3 substituents independently selected from halo, OH, NR^{N}R^{N}, and C₁₋₆alkoxy; and X¹ is null, NR^{N}R^{N}, C(O), SO₂, or OC(O). In come cases, R^{N} is H.

In some cases, Y¹ is unsubstituted C₁₋₆alkylene. In some cases, Y¹ is C₁₋₆alkylene substituted with 1-3 (or 1) substituent(s) independently selected from halo, OH, NR^{N}R^{N}, and C₁₋₆alkoxy. In some cases, Y¹ is C₁₋₆alkylene substituted with halo. In some cases, Y¹ is C₁₋₆alkylene substituted with OH. In some cases, Y¹ is C₁₋₆alkylene substituted with NR^{N}R^{N}. In some cases, Y¹ is C₁₋₆alkylene substituted with C₁₋₆alkoxy.

In some cases, X¹ is null. In some cases, X¹ is NR^{N}R^{N}, C(O), SO₂, or OC(O). In some cases, X¹ is NR^{N}R^{N}. In some cases, X¹ is C(O). In some cases, X¹ is SO₂. In some cases, X¹ is OC(O).

In some cases, A is C₅₋₈carbocyclyl or C₆₋₁₀aryl, and optionally substituted with 1-2 substituents independently selected from halo, C₁₋₆alkyl, and COO—C₁₋₆alkyl. In some cases, A is 4-12-membered heterocycle having 1-3 ring heteroatoms selected from N, O, and S, or 5-8-membered heteroaryl having 1-3 ring heteroatoms selected from N, O, and S, optionally substituted with 1-2 substituents independently selected from halo, C₁₋₆alkyl, and COO—C₁₋₆alkyl. In some cases, A is C₅₋₈carbocyclyl. In some cases, A is a 4-12-membered heterocycle having 1-3 ring heteroatoms selected from N, O, and S. In some cases, A is C₆₋₁₀aryl. In some cases, A is a 5-8-membered heteroaryl having 1-3 ring heteroatoms selected from N, O, and S. In some cases, A comprises pyridyl (e.g., 2-pyridyl).

In some cases, R⁴ and R⁸ together with the atoms to which they are attached form a 5- to 8-membered heterocycle having 1-3 ring heteroatoms selected from N, O, and S. In some cases, R⁴ and R⁸ together with the atoms to which they are attached form a 6-membered heterocycle. In some cases, R⁴ and R⁸ together with the atoms to which they are attached form a 6-membered heterocycle substituted with 1-3 R^{C}. In some cases, the 6-membered heterocycle is substituted with 1 or 2 R^{C}. In some cases, the 6-membered heterocycle is substituted with OH.

In some cases, R⁶ and R⁸ together with the atoms to which they are attached form a 5- to 8-membered heterocycle having 1-3 ring heteroatoms selected from N, O, and S. In some cases, R⁶ and R⁸ together with the atoms to which they are attached form a 5-membered heterocycle. In some cases, the heterocycle is substituted with CN, C₁₋₆ alkylene-O—C₁₋₆alkyl, C₁₋₆alkylene-O—C₁₋₆alkylene-O—C₁₋₆alkyl, C₀₋₆alkylene-C₆₋₁₀aryl, or a 6-membered heterocycle. In some cases, the heterocycle is substituted with CN. In some cases, the heterocycle is substituted with C₁₋₆alkylene-O—C₁₋₆alkyl. In some cases, the heterocycle is substituted with C₂alkylene-O—C₂alkyl. In some cases, the heterocycle is substituted with C₁₋₆alkylene-O—C₁₋₆alkylene-O—C₁₋₆alkyl. In some cases, the heterocycle is substituted with C₂alkylene-O—C₂alkylene-O—C₂alkyl. In some cases, the heterocycle is substituted with C₁₋₆alkylene-C₆₋₁₀aryl. In some cases, the heterocycle is substituted with C₀alkylene-C₆₋₁₀aryl. In some cases, the heterocycle is substituted with phenyl. In some cases, the heterocycle is substituted with a 6-membered heterocycle. In some cases, the heterocycle is substituted with morpholinyl.

In some cases, R⁷ is H. In some cases, R⁷ is C₁₋₆alkyl. In some cases, R⁷ is methyl. In some cases, R⁷ is C₅alkyl. In some cases, R⁷ is C₁₋₆alkylene-O—C₁₋₆alkyl or C₁₋₆alkylene-O—C₁₋₆alkylene-O—C₁₋₆alkyl. In some cases, R⁷ is C₁₋₆alkylene-O—C₁₋₆alkyl. In some cases, R⁷ is C₂alkylene-O—C₂alkyl. In some cases, R⁷ is C₁₋₆alkylene-O—C₁₋₆alkylene-O—C₁₋₆alkyl. In some cases, R⁷ is C₂alkylene-O—C₂alkylene-O—C₂alkyl. In some cases, R⁷ is C₁₋₆alkylene-CONH—C₁₋₆ alkyl, C₁₋₆alkylene-NHCONH—C₁₋₆alkyl, C₁₋₆alkylene-OCONH—C₁₋₆alkyl, or C₁₋₆alkylene-NHSO₂—C₁₋₆alkyl. In some cases, R⁷ is C₁₋₆alkylene-CONH—C₁₋₆alkyl. In some cases, R⁷ is C₁alkylene-CONH—C₂alkyl. In some cases, R⁷ is C₁₋₆alkylene-CON(CH₃)₂. In some cases, R⁷ is CON(CH₃)₂. In some cases, R⁷ is C₁₋₆alkylene-NHCONH—C₁₋₆alkyl. In some cases, R⁷ is C₂alkylene-NHCONH—C₂alkyl. In some cases, R⁷ is C₁₋₆alkylene-OCONH—C₁₋₆alkyl. In some cases, R⁷ is C₂alkylene-OCONH—C₂alkyl. In some cases, R⁷ is C₁₋₆alkylene-NHCOO—C₁₋₆alkyl. In some cases, R⁷ is C₂alkylene-NHCOO—C₂alkyl. In some cases, R⁷ is C₁₋₆alkylene-NHSO₂—C₁₋₆alkyl. In some cases, R⁷ is C₂alkylene-NHSO₂—C₂alkyl. In some cases, R⁷ is C₁₋₆alkylene-C₆₋₁₀aryl or C₁₋₆alkylene-5-8 membered heteroaryl having 1-3 ring heteroatoms selected from N, O, and S, and the aryl or heteroaryl is optionally substituted with 1-3 substituents independently selected from C₁₋₆alkoxy and halo. In some cases, R⁷ is C₁₋₆alkylene-C₆₋₁₀aryl wherein C₁₋₆alkylene is optionally substituted with C₃₋₅carbocyclyl. In some cases, R⁷ is C₁₋₆alkylene-C₆₋₁₀aryl wherein C₁₋₆alkylene is substituted with C₃₋₅ carbocyclyl. In some cases, R⁷ is C₁₋₆alkylene-C₆₋₁₀aryl wherein C₁₋₆alkylene is substituted with C₃carbocyclyl. In some cases, R⁷ is C₁alkylene-C₆aryl. In some cases, R⁷ is C₂alkylene-C₆aryl. In some cases, C₆₋₁₀aryl is substituted with 1 substituent selected from C₁₋₆alkoxy and halo. In some cases, C₆₋₁₀aryl is substituted with C₁₋₆alkoxy. In some cases, C₆₋₁₀aryl is substituted with halo. In some cases, C₆₋₁₀aryl is substituted with chloro. In some cases, R⁷ is C₁₋₆alkylene-5-8 membered heteroaryl having 1-3 ring heteroatoms selected from N, O, and S, and the heteroaryl is optionally substituted with 1-3 substituents independently selected from C₁₋₆alkoxy and halo. In some cases, R⁷ is C₂alkylene-pyridyl, optionally substituted with 1-3 substituents independently selected from C₁₋₆alkoxy and halo.

In some cases, R⁸ is H. In some cases, R⁸ is C₁₋₆alkyl. In some cases, R⁸ is methyl.

In some cases, R⁷ and R⁸ together with the nitrogen to which they are attached form a 5-12 membered heterocycle. In some cases, R⁷ and R⁸ together with the atoms to which they are attached form a 5 membered heterocycle. In some cases, R⁷ and R⁸ together with the nitrogen to which they are attached form a 6 membered heterocycle. In some cases, the 6 membered heterocycle is substituted with C₆₋₁₀aryl. In some cases, the 6 membered heterocycle is substituted with phenyl. In some cases, the 5-12 membered heterocycle is substituted with halo. In some cases, the 5-12 membered heterocycle is substituted with COO—C₁₋₆alkyl. In some cases, R⁷ and R⁸ together with the nitrogen to which they are attached form

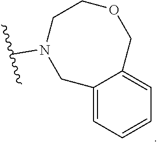 [SMILES: *N1CCOCc2ccccc2C1]

Also provided herein are compounds of Formula (II), and pharmaceutically acceptable salts thereof:

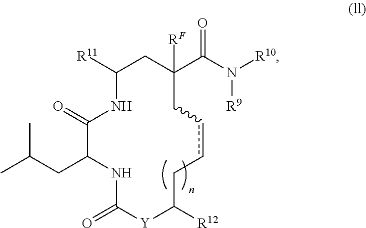

wherein

Y is O or a bond;

R^{F} is H, or

R^{F} and R¹⁰ together with the atoms to which they are attached form a five-membered heterocycle;

R⁹ is C₁₋₆alkyl, or C₁₋₆alkylene-C₆₋₁₀aryl, wherein C₆₋₁₀aryl is optionally substituted with 1-3 substituents independently selected from C₁₋₃alkoxy and halo;

R¹⁰ is H or C₁₋₆alkyl;

R¹¹ is C₁₋₆alkylene-OH, C₁₋₆alkylene-OH substituted with PO(OCH₂CH₂)₂, CHO, or (CO)₁₋₂NR¹³R¹⁴; or

R¹⁰ and R¹¹ together with the atoms to which they are attached form a six-membered heterocycle optionally substituted with 1-3 substituents independently selected from OH and CONR¹³R¹⁴;

R¹² is H, C₁₋₈alkyl, or C₁₋₆alkylene-C₆₋₁₀aryl, wherein C₆₋₁₀aryl is optionally substituted with 1-3 substituents independently selected from C₁₋₃alkoxy and halo;

R¹³ and R¹⁴ are each independently H, C₁₋₆alkyl, or C₁₋₆alkoxy;

[symbol] indicates a single or a double bond; and

n is 1-3.

In some cases, the compound of Formula (II) has a structure of Formula (IIA), (IIB), (IIC), or (IID):

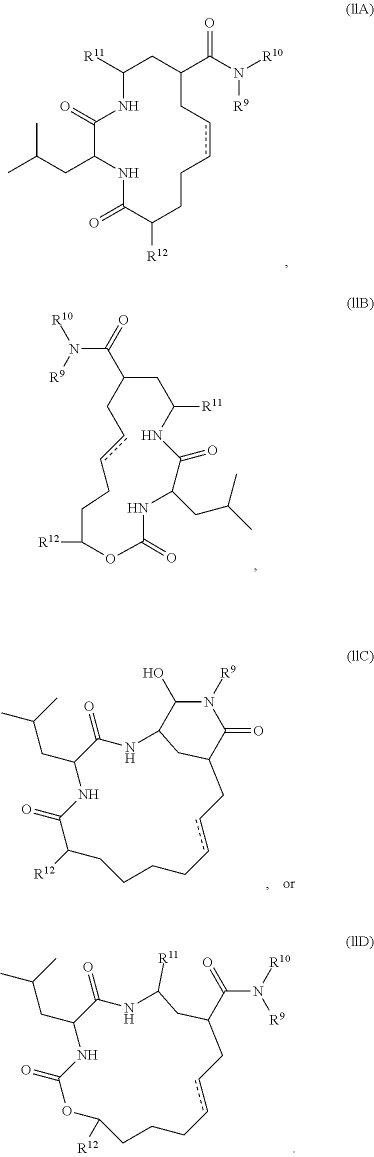

In some cases, Y is O. In some cases, Y is a bond.

In some cases, n is 1. In some cases, n is 2. In some cases, n is 3.

In some cases, R^{F} is H.

In some cases, R^{F} and R¹⁰ together with the atoms to which they are attached form a five-membered heterocycle.

In some cases, R⁹ is C₁₋₆alkyl. In some cases, R⁹ is methylR⁹ is C₁₋₆alkylene-C₆₋₁₀aryl. In some cases, R⁹ is C₁alkylene-C₆₋₁₀aryl. In some cases, C₆₋₁₀aryl is substituted with 1-3 substituents independently selected from C₁₋₃alkoxy and halo. In some cases, C₆₋₁₀aryl is substituted with 1-3 C₁₋₃alkoxy. In some cases, C₆₋₁₀aryl is substituted with 1-3 halo. In some cases, R⁹ is benzyl.

In some cases, R¹⁰ is H. In some cases, R¹⁰ is C₁₋₆alkyl. In some cases, R¹⁰ is methyl.

In some cases, R¹⁰ and R¹¹ together with the atoms to which they are attached form a six-membered heterocycle. In some cases, the six-membered heterocycle is substituted with 1-3 substituents independently selected from OH and CONR¹³R¹⁴. In some cases, the six-membered heterocycle is substituted with OH.

In some cases, R¹¹ is C₁₋₆alkylene-OH. In some cases, R¹¹ is C₁₋₆alkylene-OH substituted with PO(OCH₂CH₂)₂. In some cases, R¹¹ is CHO. In some cases, R¹¹ is CONR¹³R¹⁴. In some cases, R¹¹ is

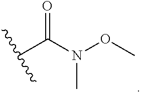 [SMILES: *C(=O)N(C)OC]

In some cases, one of R¹³ and R¹⁴ is H. In some cases, R¹³ is C₁₋₆alkyl. In some cases, R¹³ is methyl. In some cases, R¹⁴ is C₁₋₆alkoxy. In some cases, R¹⁴ is methoxy.

In some cases, R¹² is H. In some cases, R¹² is C₁₋₈alkyl. In some cases, R¹² is C₇alkyl. In some cases, R¹² is C₈alkyl. In some cases, R¹² is C₁₋₆alkylene-C₆₋₁₀aryl. In some cases, R¹² is unsubstituted C₁₋₆alkylene-C₆₋₁₀aryl. In some cases, R¹² is benzyl. In some cases, R¹² is C₁₋₆alkylene-C₆₋₁₀aryl substituted with 1-3 substituents independently selected from C₁₋₃alkoxy and halo.

In some cases, [symbol] indicates a single bond. In some cases, [symbol] indicates a double bond. In some cases, the double bond is cis. In some cases, the double bond is trans.

It is understood that selections of values of each variable are those that result in the formation of stable or chemically feasible compounds.

Specific compounds contemplated include compounds in the following Tables. Compounds showing particular stereocenters indicate at least a relative stereoisomerism. Compounds having a chiral center without indication of a particular stereoisomerism indicate a mixture of stereocenters at that chiral center.

The compound can be a compound as listed in Table A, or a pharmaceutically acceptable salt thereof.

TABLE A

| Compound | |
|---|---|
| no. | Structure |
| A1 | 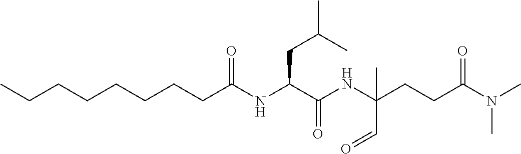 [SMILES: CCCCCCCCC(=O)N[C@@H](CC(C)C)C(=O)NC(C)(C=O)CCC(=O)N(C)C] |
| A2 | 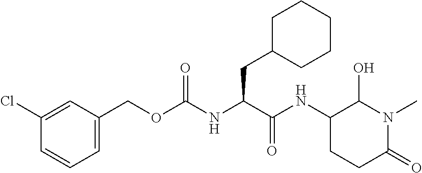 [SMILES: CN1C(=O)CCC(NC(=O)[C@H](CC2CCCCC2)NC(=O)OCc2cccc(Cl)c2)C1O] |
| A3 | 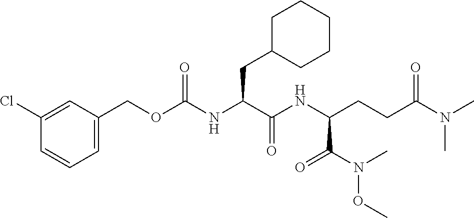 [SMILES: CON(C)C(=O)[C@H](CCC(=O)N(C)C)NC(=O)[C@H](CC1CCCCC1)NC(=O)OCc1cccc(Cl)c1] |
| A4 | 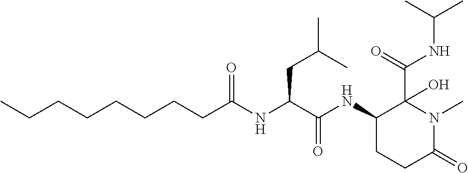 [SMILES: CCCCCCCCC(=O)N[C@@H](CC(C)C)C(=O)N[C@@H]1CCC(=O)N(C)C1(O)C(=O)NC(C)C] |
| A5 | 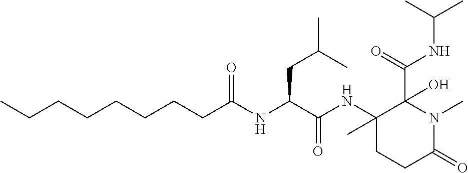 [SMILES: CCCCCCCCC(=O)N[C@@H](CC(C)C)C(=O)NC1(C)CCC(=O)N(C)C1(O)C(=O)NC(C)C] |
| A6 | 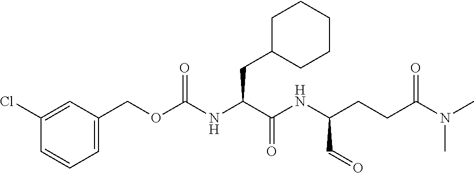 [SMILES: CN(C)C(=O)CC[C@@H](C=O)NC(=O)[C@H](CC1CCCCC1)NC(=O)OCc1cccc(Cl)c1] |
| A7 | 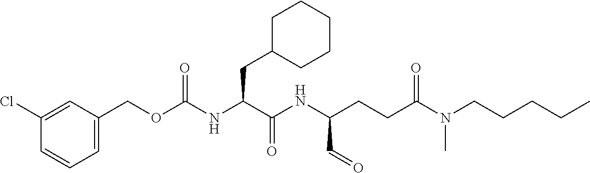 [SMILES: CCCCCN(C)C(=O)CC[C@@H](C=O)NC(=O)[C@H](CC1CCCCC1)NC(=O)OCc1cccc(Cl)c1] |
| A8 | 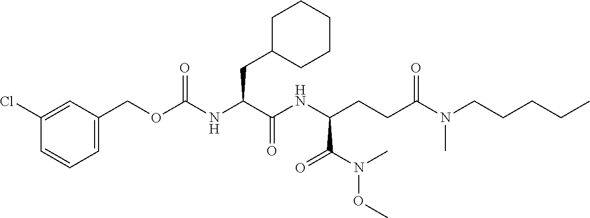 [SMILES: CCCCCN(C)C(=O)CC[C@H](NC(=O)[C@H](CC1CCCCC1)NC(=O)OCc1cccc(Cl)c1)C(=O)N(C)OC] |
| A9 | 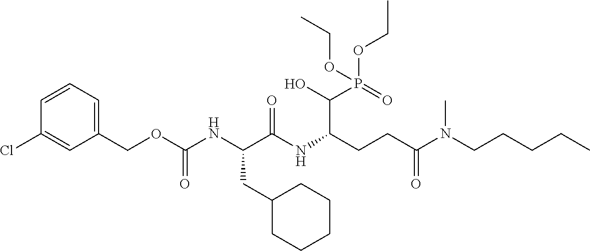 [SMILES: CCCCCN(C)C(=O)CC[C@H](NC(=O)[C@H](CC1CCCCC1)NC(=O)OCc1cccc(Cl)c1)C(O)P(=O)(OCC)OCC] |
| A10 | 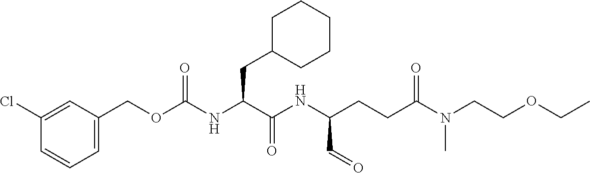 [SMILES: CCOCCN(C)C(=O)CC[C@@H](C=O)NC(=O)[C@H](CC1CCCCC1)NC(=O)OCc1cccc(Cl)c1] |
| A11 | 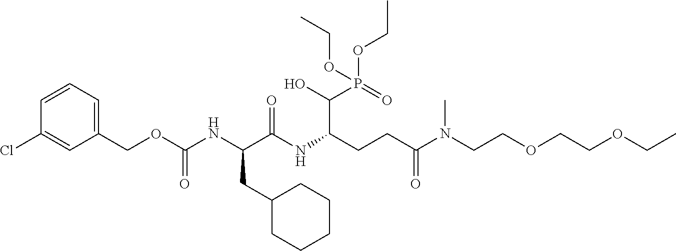 [SMILES: CCOCCOCCN(C)C(=O)CC[C@H](NC(=O)[C@@H](CC1CCCCC1)NC(=O)OCc1cccc(Cl)c1)C(O)P(=O)(OCC)OCC] |
| A12 | 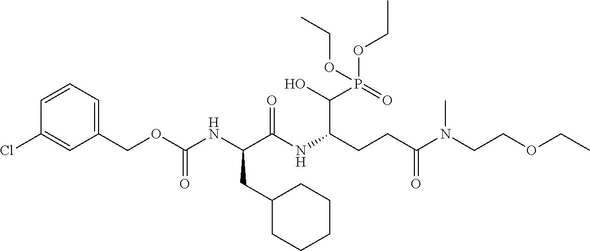 [SMILES: CCOCCN(C)C(=O)CC[C@H](NC(=O)[C@@H](CC1CCCCC1)NC(=O)OCc1cccc(Cl)c1)C(O)P(=O)(OCC)OCC] |
| A13 | 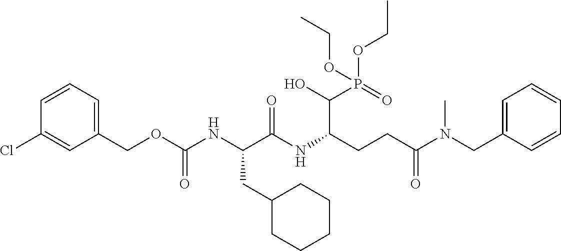 [SMILES: CCOP(=O)(OCC)C(O)[C@H](CCC(=O)N(C)Cc1ccccc1)NC(=O)[C@H](CC1CCCCC1)NC(=O)OCc1cccc(Cl)c1] |
| A14 | 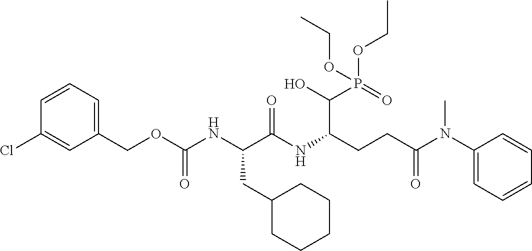 [SMILES: CCOP(=O)(OCC)C(O)[C@H](CCC(=O)N(C)c1ccccc1)NC(=O)[C@H](CC1CCCCC1)NC(=O)OCc1cccc(Cl)c1] |
| A15 | 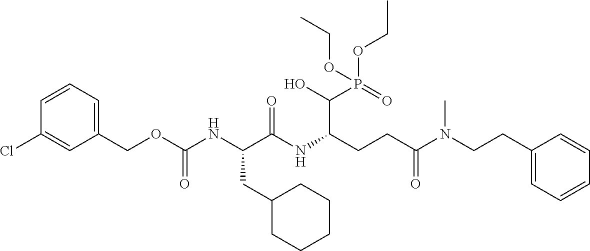 [SMILES: CCOP(=O)(OCC)C(O)[C@H](CCC(=O)N(C)CCc1ccccc1)NC(=O)[C@H](CC1CCCCC1)NC(=O)OCc1cccc(Cl)c1] |
| A16 | 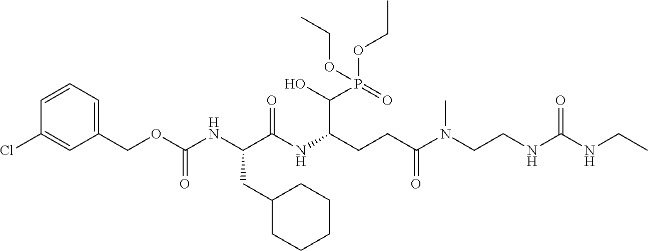 [SMILES: CCNC(=O)NCCN(C)C(=O)CC[C@H](NC(=O)[C@H](CC1CCCCC1)NC(=O)OCc1cccc(Cl)c1)C(O)P(=O)(OCC)OCC] |
| A17 | 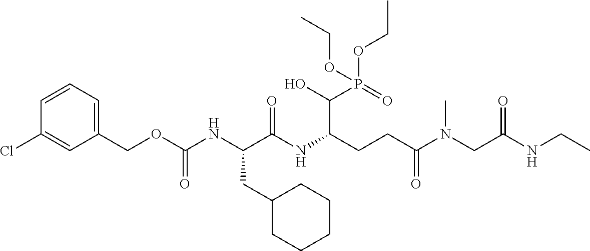 [SMILES: CCNC(=O)CN(C)C(=O)CC[C@H](NC(=O)[C@H](CC1CCCCC1)NC(=O)OCc1cccc(Cl)c1)C(O)P(=O)(OCC)OCC] |
| A18 | 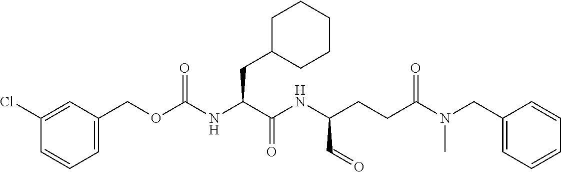 [SMILES: CN(Cc1ccccc1)C(=O)CC[C@@H](C=O)NC(=O)[C@H](CC1CCCCC1)NC(=O)OCc1cccc(Cl)c1] |
| A19 | 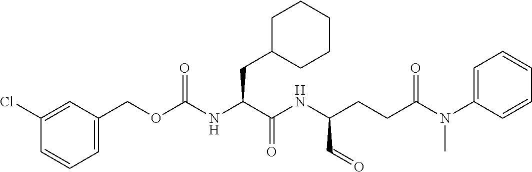 [SMILES: CN(C(=O)CC[C@@H](C=O)NC(=O)[C@H](CC1CCCCC1)NC(=O)OCc1cccc(Cl)c1)c1ccccc1] |
| A20 | 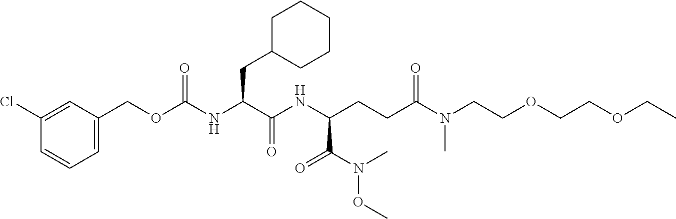 [SMILES: CCOCCOCCN(C)C(=O)CC[C@H](NC(=O)[C@H](CC1CCCCC1)NC(=O)OCc1cccc(Cl)c1)C(=O)N(C)OC] |
| A21 | 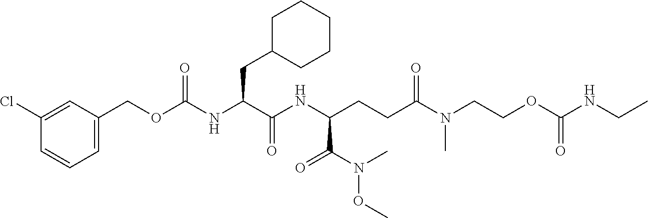 [SMILES: CCNC(=O)OCCN(C)C(=O)CC[C@H](NC(=O)[C@H](CC1CCCCC1)NC(=O)OCc1cccc(Cl)c1)C(=O)N(C)OC] |
| A22 | 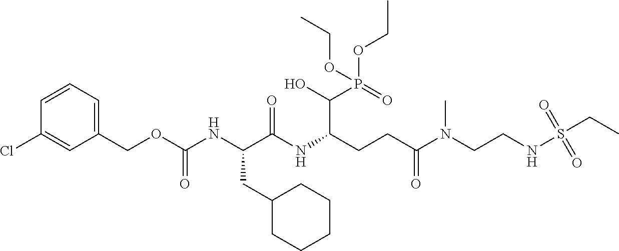 [SMILES: CCOP(=O)(OCC)C(O)[C@H](CCC(=O)N(C)CCNS(=O)(=O)CC)NC(=O)[C@H](CC1CCCCC1)NC(=O)OCc1cccc(Cl)c1] |
| A23 | 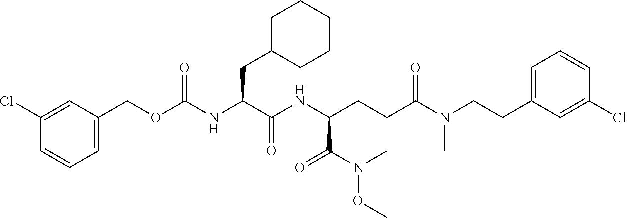 [SMILES: CON(C)C(=O)[C@H](CCC(=O)N(C)CCc1cccc(Cl)c1)NC(=O)[C@H](CC1CCCCC1)NC(=O)OCc1cccc(Cl)c1] |
| A24 | 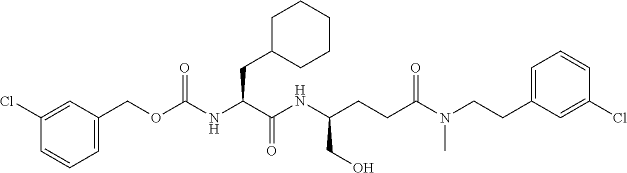 [SMILES: CN(CCc1cccc(Cl)c1)C(=O)CC[C@@H](CO)NC(=O)[C@H](CC1CCCCC1)NC(=O)OCc1cccc(Cl)c1] |
| A25 | 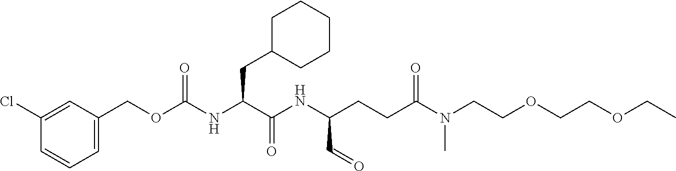 [SMILES: CCOCCOCCN(C)C(=O)CC[C@@H](C=O)NC(=O)[C@H](CC1CCCCC1)NC(=O)OCc1cccc(Cl)c1] |
| A26 | 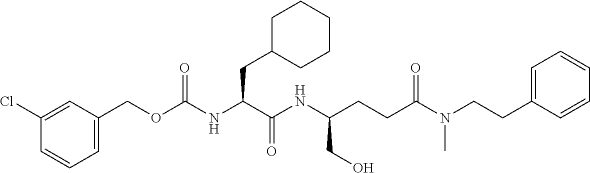 [SMILES: CN(CCc1ccccc1)C(=O)CC[C@@H](CO)NC(=O)[C@H](CC1CCCCC1)NC(=O)OCc1cccc(Cl)c1] |
| A27 | 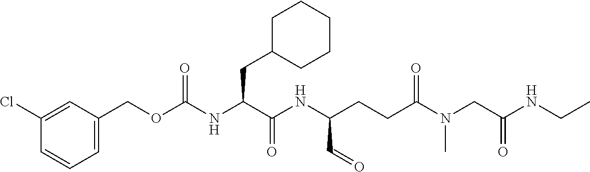 [SMILES: CCNC(=O)CN(C)C(=O)CC[C@@H](C=O)NC(=O)[C@H](CC1CCCCC1)NC(=O)OCc1cccc(Cl)c1] |
| A28 | 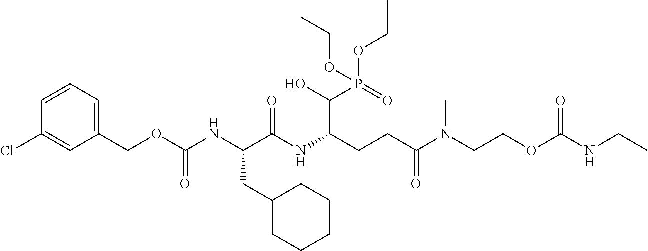 [SMILES: CCNC(=O)OCCN(C)C(=O)CC[C@H](NC(=O)[C@H](CC1CCCCC1)NC(=O)OCc1cccc(Cl)c1)C(O)P(=O)(OCC)OCC] |
| A29 | 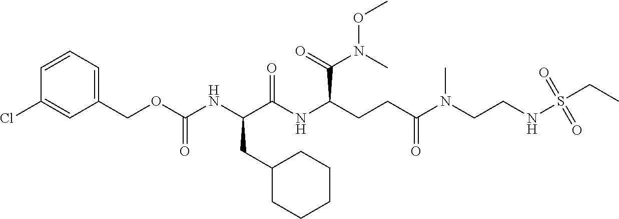 [SMILES: CCS(=O)(=O)NCCN(C)C(=O)CC[C@@H](NC(=O)[C@@H](CC1CCCCC1)NC(=O)OCc1cccc(Cl)c1)C(=O)N(C)OC] |
| A30 | 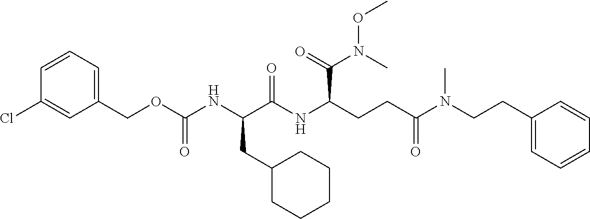 [SMILES: CON(C)C(=O)[C@@H](CCC(=O)N(C)CCc1ccccc1)NC(=O)[C@@H](CC1CCCCC1)NC(=O)OCc1cccc(Cl)c1] |
| A31 | 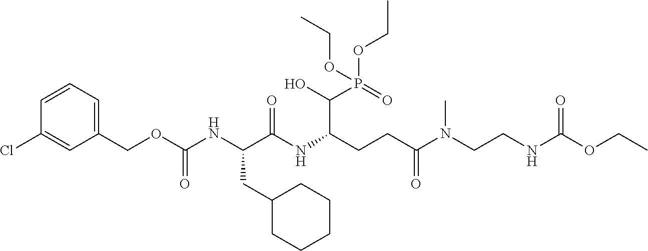 [SMILES: CCOC(=O)NCCN(C)C(=O)CC[C@H](NC(=O)[C@H](CC1CCCCC1)NC(=O)OCc1cccc(Cl)c1)C(O)P(=O)(OCC)OCC] |
| A32 | 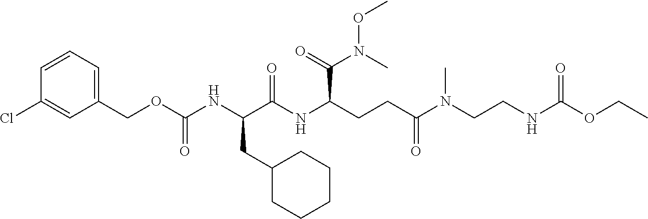 [SMILES: CCOC(=O)NCCN(C)C(=O)CC[C@@H](NC(=O)[C@@H](CC1CCCCC1)NC(=O)OCc1cccc(Cl)c1)C(=O)N(C)OC] |
| A33 | 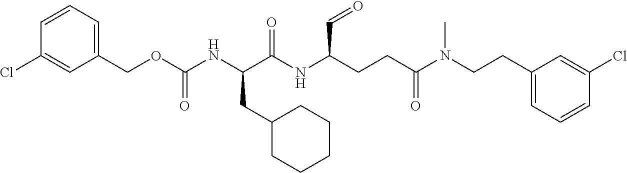 [SMILES: CN(CCc1cccc(Cl)c1)C(=O)CC[C@H](C=O)NC(=O)[C@@H](CC1CCCCC1)NC(=O)OCc1cccc(Cl)c1] |
| A34 | 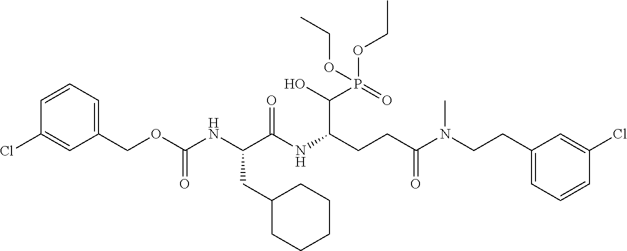 [SMILES: CCOP(=O)(OCC)C(O)[C@H](CCC(=O)N(C)CCc1cccc(Cl)c1)NC(=O)[C@H](CC1CCCCC1)NC(=O)OCc1cccc(Cl)c1] |
| A35 | 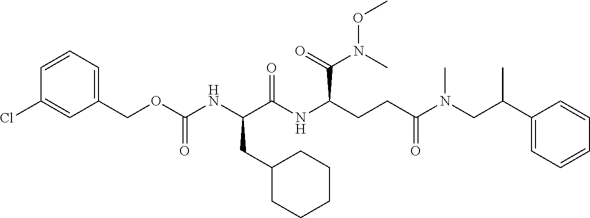 [SMILES: CON(C)C(=O)[C@@H](CCC(=O)N(C)CC(C)c1ccccc1)NC(=O)[C@@H](CC1CCCCC1)NC(=O)OCc1cccc(Cl)c1] |
| A36 | 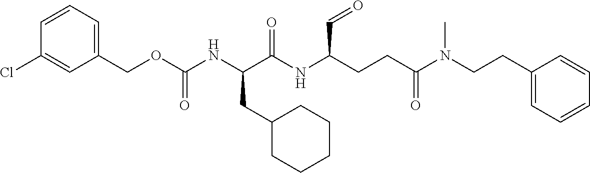 [SMILES: CN(CCc1ccccc1)C(=O)CC[C@H](C=O)NC(=O)[C@@H](CC1CCCCC1)NC(=O)OCc1cccc(Cl)c1] |
| A37 | 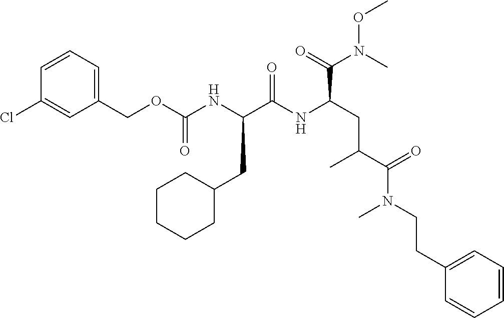 [SMILES: CON(C)C(=O)[C@@H](CC(C)C(=O)N(C)CCc1ccccc1)NC(=O)[C@@H](CC1CCCCC1)NC(=O)OCc1cccc(Cl)c1] |
| A38 | 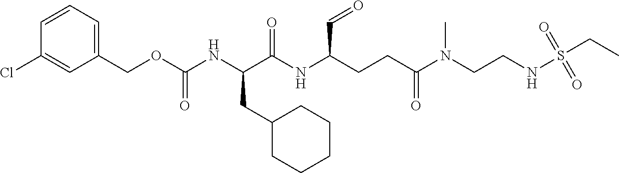 [SMILES: CCS(=O)(=O)NCCN(C)C(=O)CC[C@H](C=O)NC(=O)[C@@H](CC1CCCCC1)NC(=O)OCc1cccc(Cl)c1] |
| A39 | 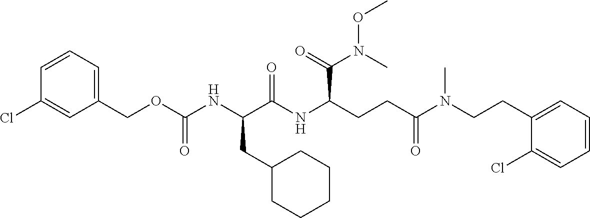 [SMILES: CON(C)C(=O)[C@@H](CCC(=O)N(C)CCc1ccccc1Cl)NC(=O)[C@@H](CC1CCCCC1)NC(=O)OCc1cccc(Cl)c1] |
| A40 | 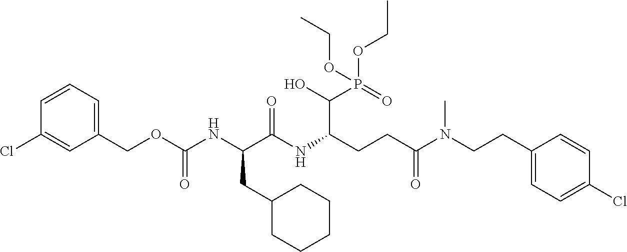 [SMILES: CCOP(=O)(OCC)C(O)[C@H](CCC(=O)N(C)CCc1ccc(Cl)cc1)NC(=O)[C@@H](CC1CCCCC1)NC(=O)OCc1cccc(Cl)c1] |
| A41 | 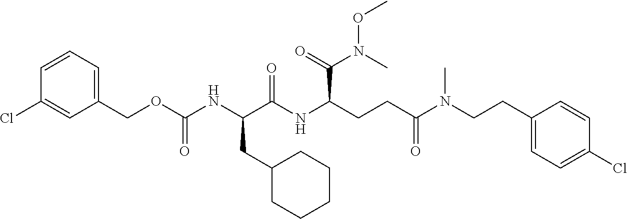 [SMILES: CON(C)C(=O)[C@@H](CCC(=O)N(C)CCc1ccc(Cl)cc1)NC(=O)[C@@H](CC1CCCCC1)NC(=O)OCc1cccc(Cl)c1] |
| A42 | 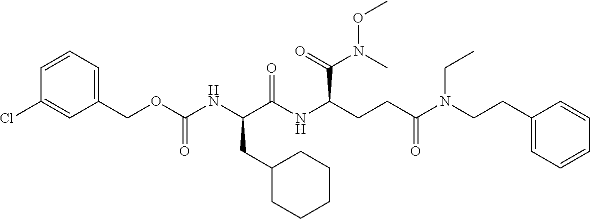 [SMILES: CCN(CCc1ccccc1)C(=O)CC[C@@H](NC(=O)[C@@H](CC1CCCCC1)NC(=O)OCc1cccc(Cl)c1)C(=O)N(C)OC] |
| A43 | 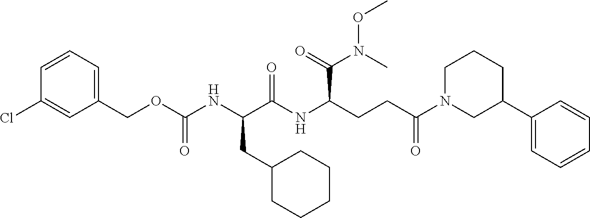 [SMILES: CON(C)C(=O)[C@@H](CCC(=O)N1CCCC(c2ccccc2)C1)NC(=O)[C@@H](CC1CCCCC1)NC(=O)OCc1cccc(Cl)c1] |
| A44 | 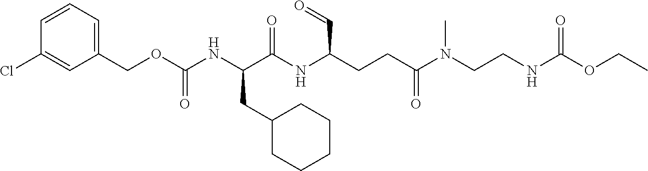 [SMILES: CCOC(=O)NCCN(C)C(=O)CC[C@H](C=O)NC(=O)[C@@H](CC1CCCCC1)NC(=O)OCc1cccc(Cl)c1] |
| A45 | 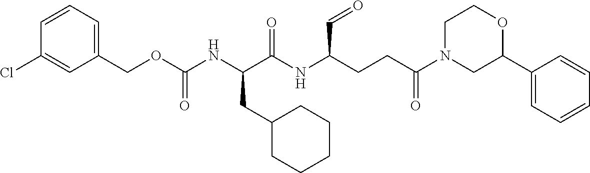 [SMILES: O=C[C@@H](CCC(=O)N1CCOC(c2ccccc2)C1)NC(=O)[C@@H](CC1CCCCC1)NC(=O)OCc1cccc(Cl)c1] |
| A46 | 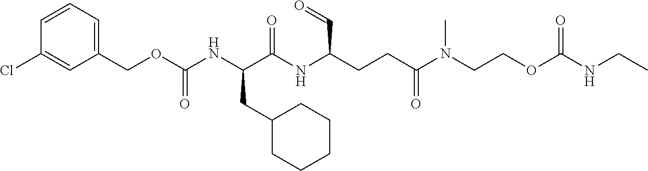 [SMILES: CCNC(=O)OCCN(C)C(=O)CC[C@H](C=O)NC(=O)[C@@H](CC1CCCCC1)NC(=O)OCc1cccc(Cl)c1] |
| A47 | 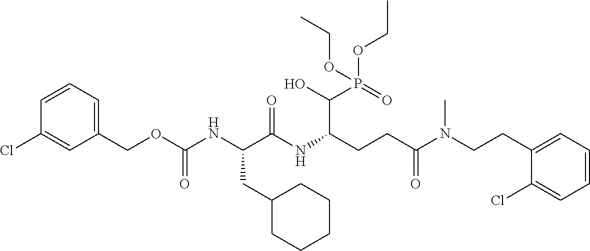 [SMILES: CCOP(=O)(OCC)C(O)[C@H](CCC(=O)N(C)CCc1ccccc1Cl)NC(=O)[C@H](CC1CCCCC1)NC(=O)OCc1cccc(Cl)c1] |
| A48 | 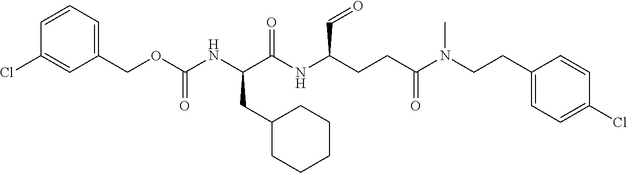 [SMILES: CN(CCc1ccc(Cl)cc1)C(=O)CC[C@H](C=O)NC(=O)[C@@H](CC1CCCCC1)NC(=O)OCc1cccc(Cl)c1] |
| A49 | 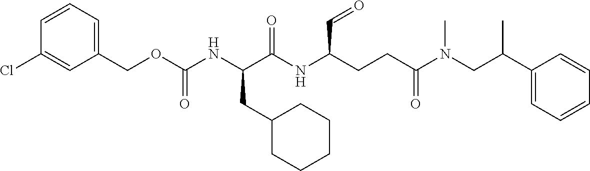 [SMILES: CC(CN(C)C(=O)CC[C@H](C=O)NC(=O)[C@@H](CC1CCCCC1)NC(=O)OCc1cccc(Cl)c1)c1ccccc1] |
| A50 | 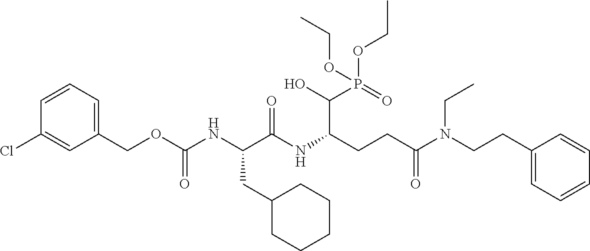 [SMILES: CCOP(=O)(OCC)C(O)[C@H](CCC(=O)N(CC)CCc1ccccc1)NC(=O)[C@H](CC1CCCCC1)NC(=O)OCc1cccc(Cl)c1] |
| A51 | 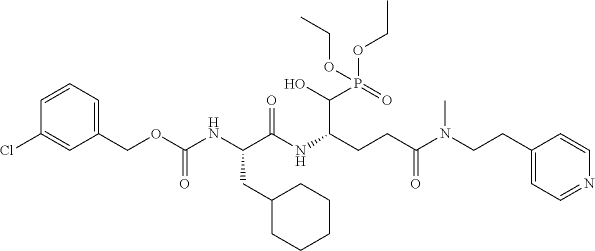 [SMILES: CCOP(=O)(OCC)C(O)[C@H](CCC(=O)N(C)CCc1ccncc1)NC(=O)[C@H](CC1CCCCC1)NC(=O)OCc1cccc(Cl)c1] |
| A52 | 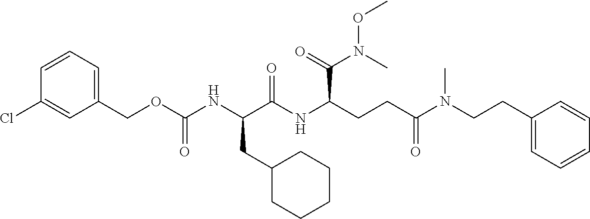 [SMILES: CON(C)C(=O)[C@@H](CCC(=O)N(C)CCc1ccccc1)NC(=O)[C@@H](CC1CCCCC1)NC(=O)OCc1cccc(Cl)c1] |
| A53 | 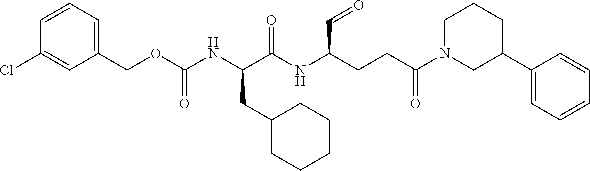 [SMILES: O=C[C@@H](CCC(=O)N1CCCC(c2ccccc2)C1)NC(=O)[C@@H](CC1CCCCC1)NC(=O)OCc1cccc(Cl)c1] |
| A54 | 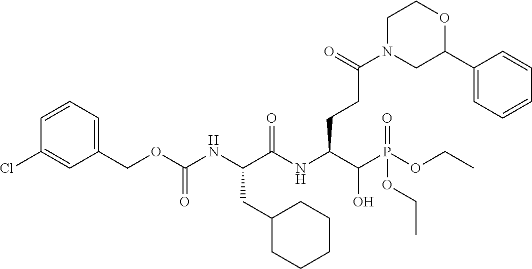 [SMILES: CCOP(=O)(OCC)C(O)[C@H](CCC(=O)N1CCOC(c2ccccc2)C1)NC(=O)[C@H](CC1CCCCC1)NC(=O)OCc1cccc(Cl)c1] |
| A55 | 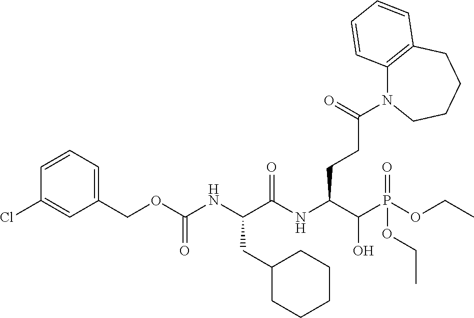 [SMILES: CCOP(=O)(OCC)C(O)[C@H](CCC(=O)N1CCCCc2ccccc21)NC(=O)[C@H](CC1CCCCC1)NC(=O)OCc1cccc(Cl)c1] |
| A56 | 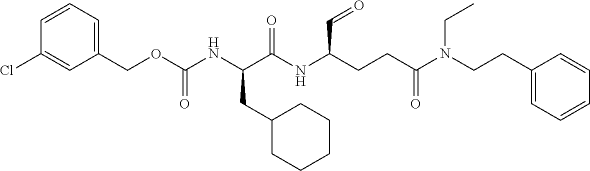 [SMILES: CCN(CCc1ccccc1)C(=O)CC[C@H](C=O)NC(=O)[C@@H](CC1CCCCC1)NC(=O)OCc1cccc(Cl)c1] |
| A57 | 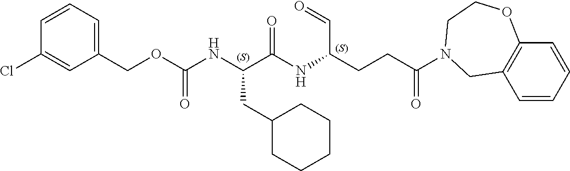 |
| A58 | 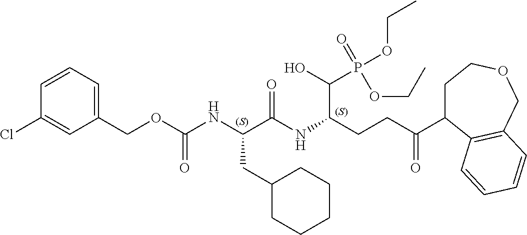 |
| A59 | 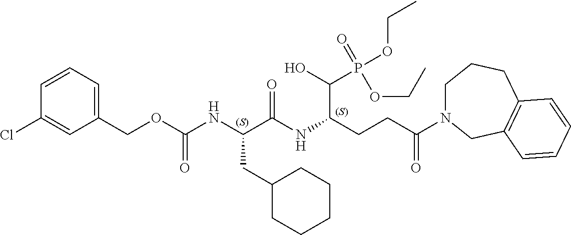 |
| A60 | 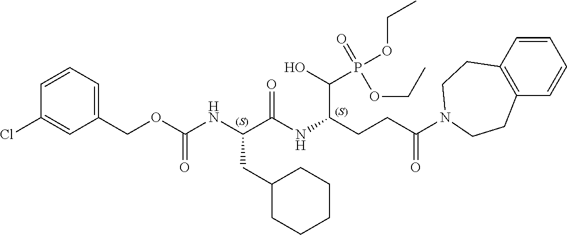 |
| A61 | 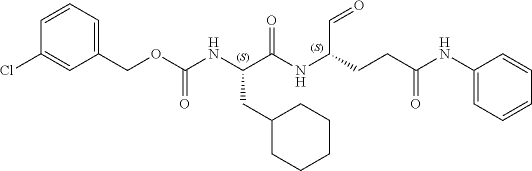 |
| A62 | 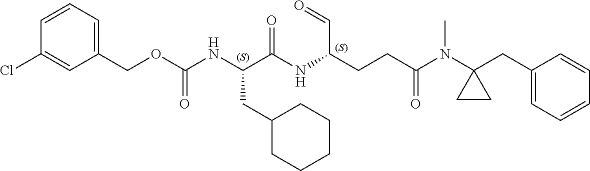 |
| A63 | 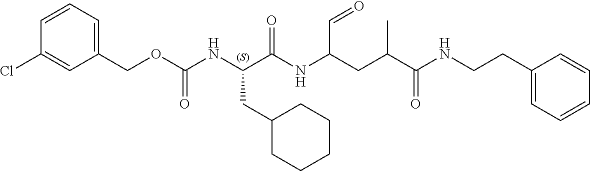 |
| A64 | 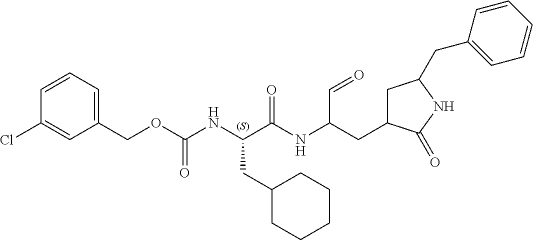 |
| A65 | 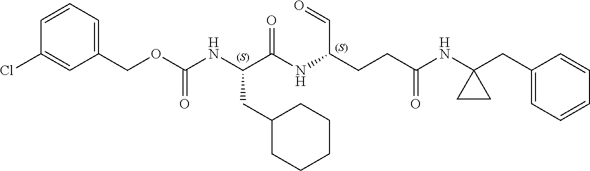 |
| A66 | 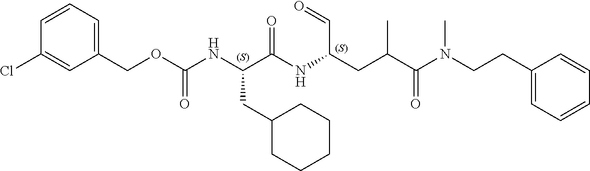 |
| A67 | 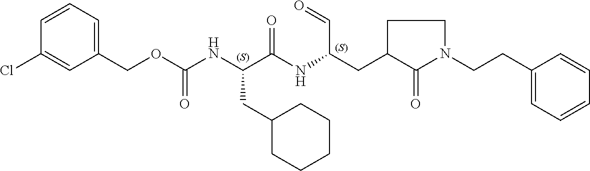 |
| A68 | 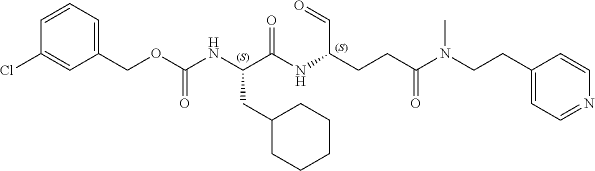 |
| A69 | 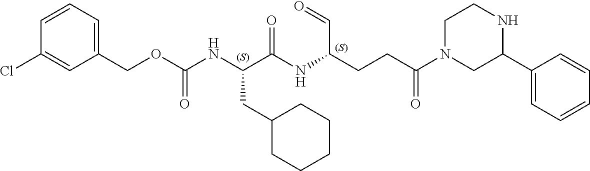 |
| A70 | 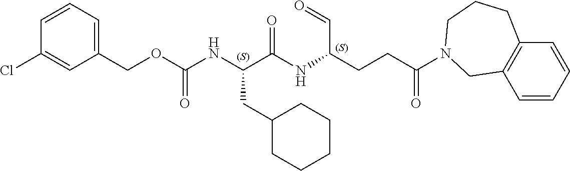 |
| A71 | 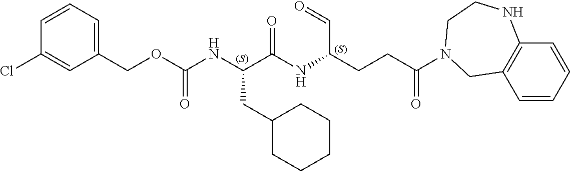 |
| A72 | 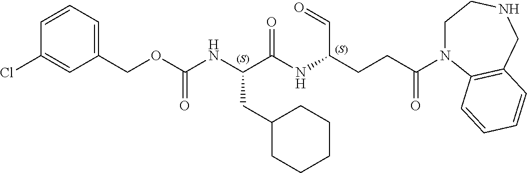 |
| A73 | 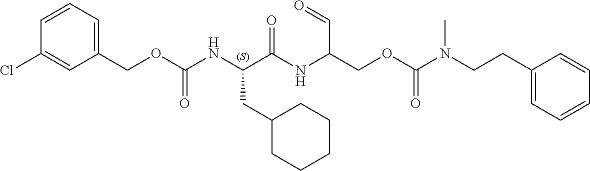 |
| A74 | 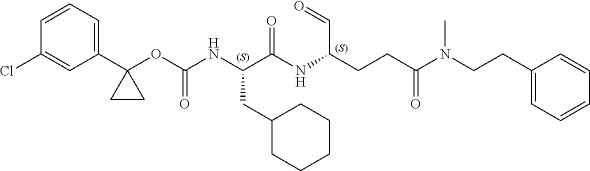 |
| A75 | 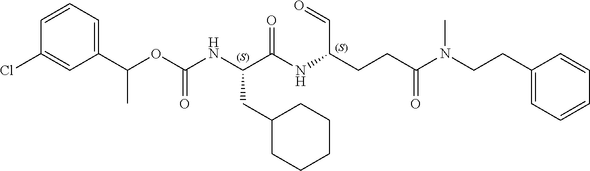 |
| A76 | 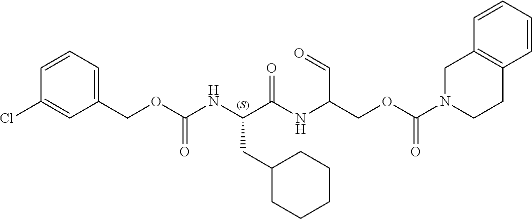 |
| A77 | 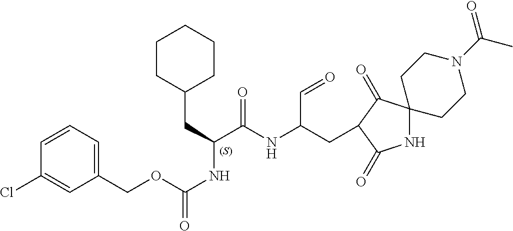 |
| A78 | 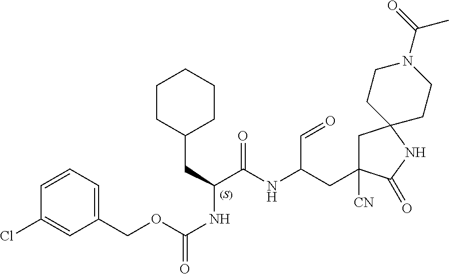 |
| A79 | 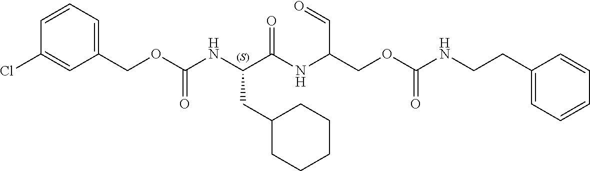 |
| A80 | 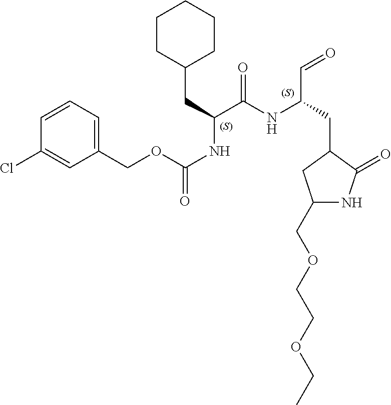 |
| A81 | 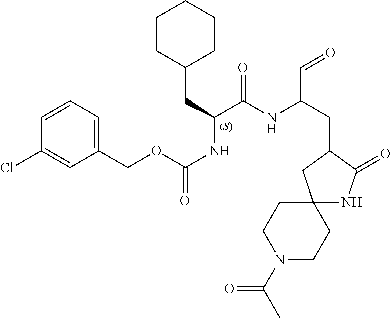 |
| A82 | 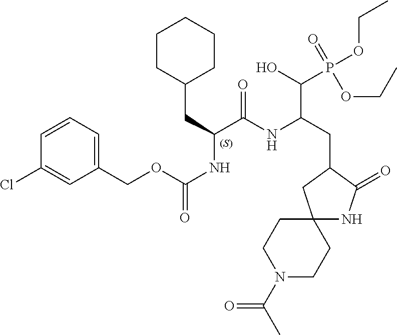 |
| A83 | 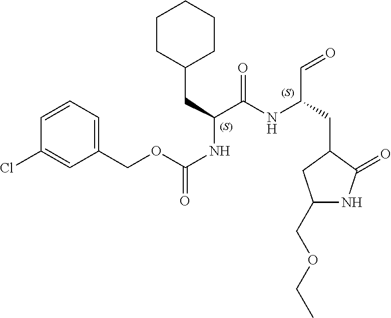 |
| A84 | 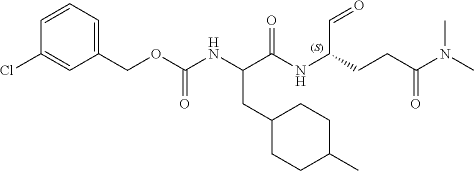 |
| A85 | 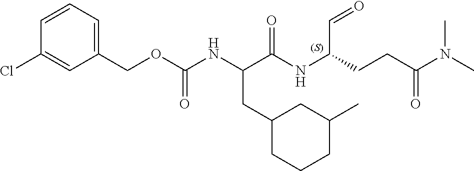 |
| A86 | 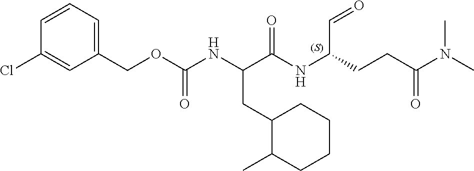 |
| A87 | 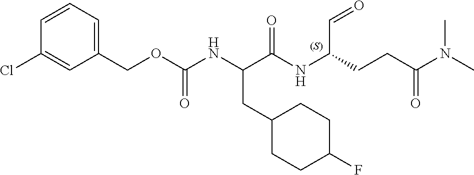 |
| A88 | 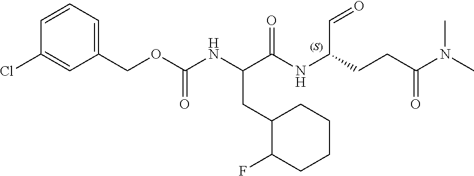 |
| A89 | 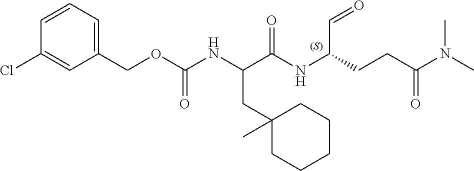 |
| A90 | 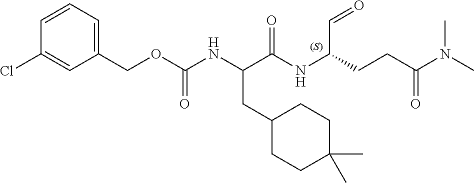 |
| A91 | 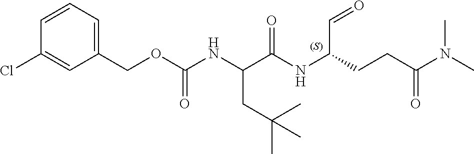 |
| A92 | 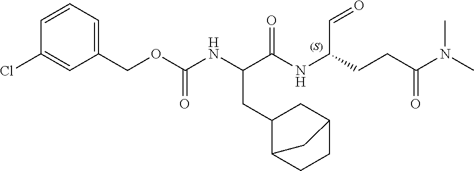 |
| A93 | 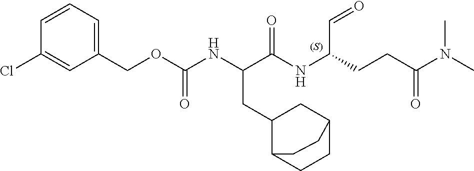 |
| A94 | 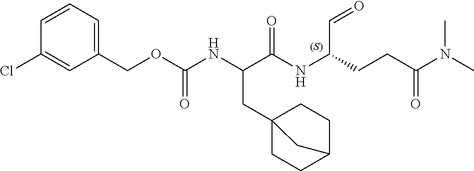 |
| A95 | 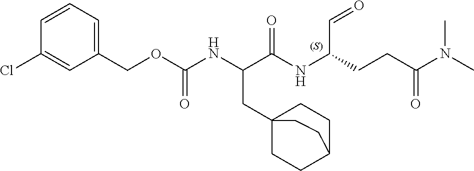 |
| A96 | 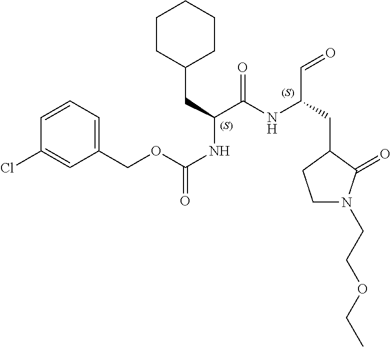 |
| A97 | 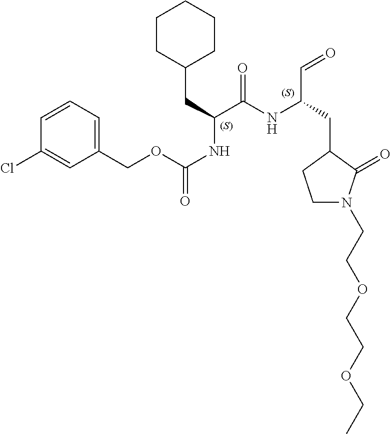 |
| A98 | 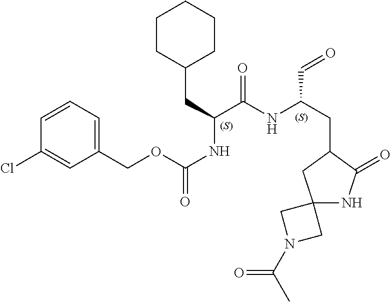 |
| A99 | 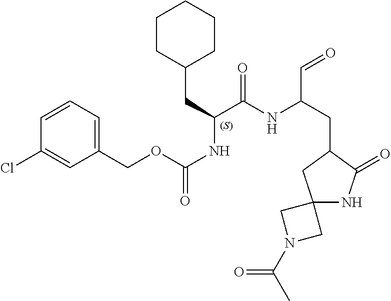 |
| A100 | 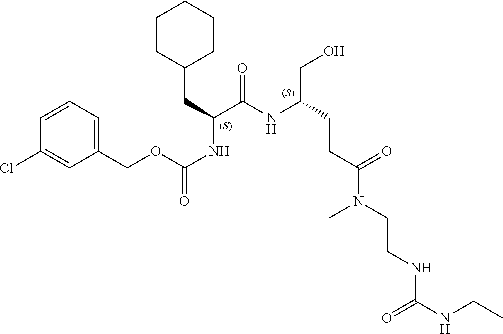 |
| A101 | 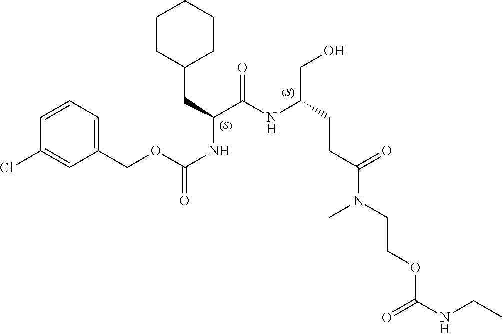 |
| A102 | 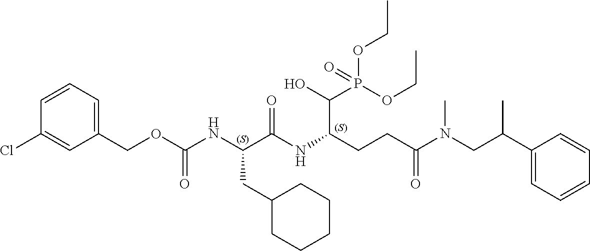 |
| A104 | 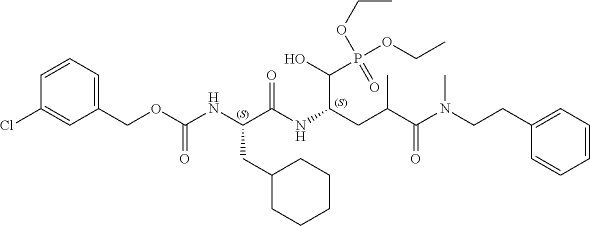 |
| A105 | 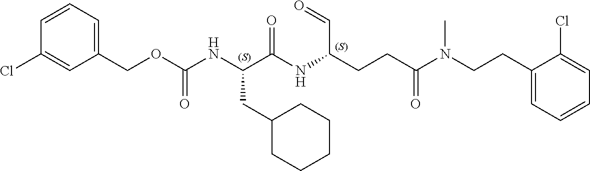 |
| A106 | 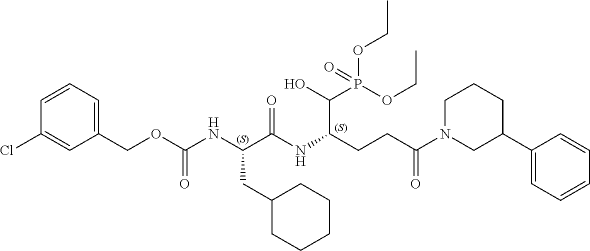 |
| A107 | 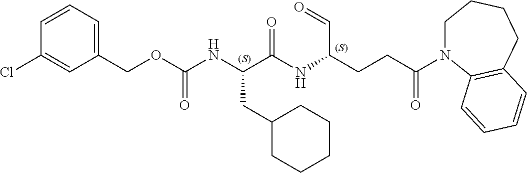 |
| A108 | 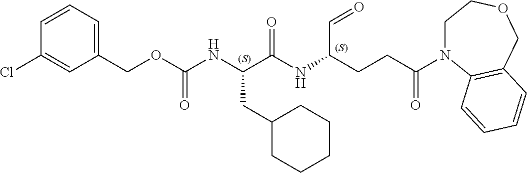 |
| A109 | 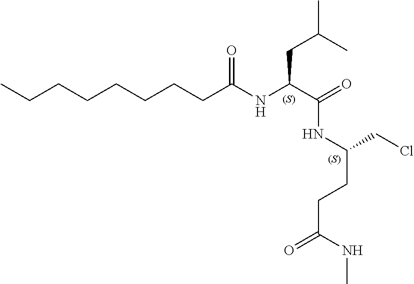 |
| A110 | 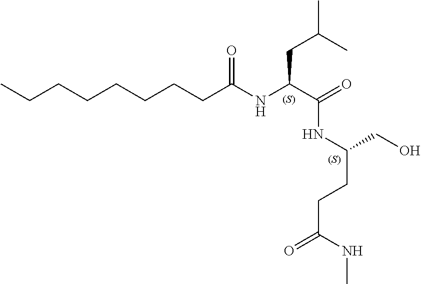 |
| A111 | 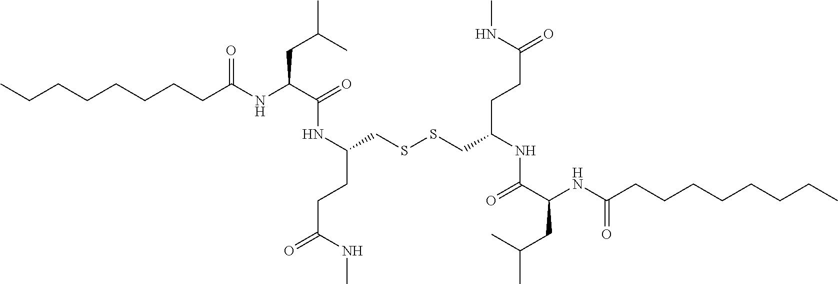 [SMILES: CCCCCCCCC(=O)N[C@@H](CC(C)C)C(=O)N[C@@H](CCC(=O)NC)CSSC[C@H](CCC(=O)NC)NC(=O)[C@H](CC(C)C)NC(=O)CCCCCCCC] |
| A112 | 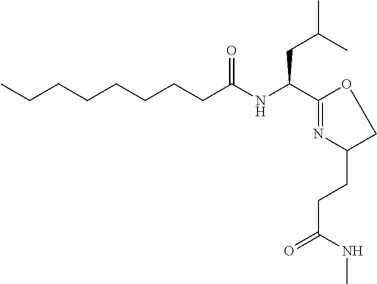 [SMILES: CCCCCCCCC(=O)N[C@@H](CC(C)C)C1=NC(CCC(=O)NC)CO1] |
| A113 | 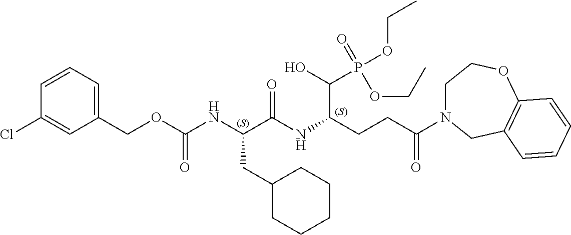 |
| A114 | 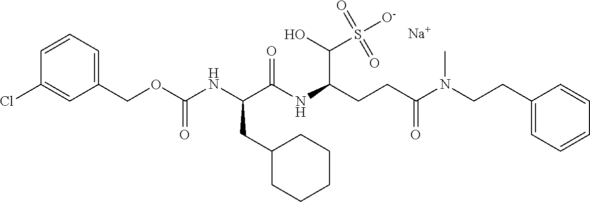 [SMILES: CN(CCc1ccccc1)C(=O)CC[C@@H](NC(=O)[C@@H](CC1CCCCC1)NC(=O)OCc1cccc(Cl)c1)C(O)S(=O)(=O)[O-] ; [Na+]] |
| A115 | 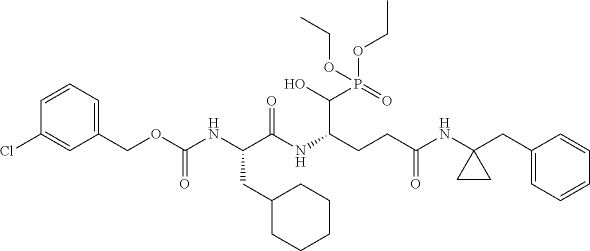 [SMILES: CCOP(=O)(OCC)C(O)[C@H](CCC(=O)NC1(Cc2ccccc2)CC1)NC(=O)[C@H](CC1CCCCC1)NC(=O)OCc1cccc(Cl)c1] |
| A116 | 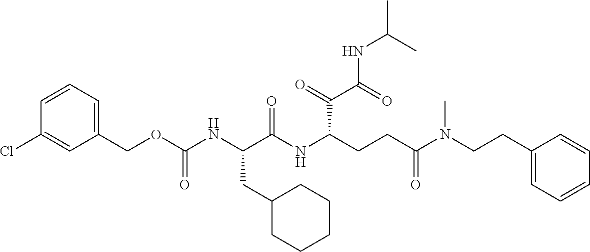 [SMILES: CC(C)NC(=O)C(=O)[C@H](CCC(=O)N(C)CCc1ccccc1)NC(=O)[C@H](CC1CCCCC1)NC(=O)OCc1cccc(Cl)c1] |
| A117 | 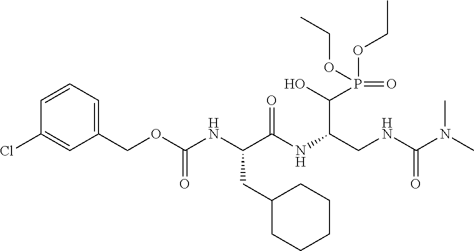 [SMILES: CCOP(=O)(OCC)C(O)[C@H](CNC(=O)N(C)C)NC(=O)[C@H](CC1CCCCC1)NC(=O)OCc1cccc(Cl)c1] |
| A118 | 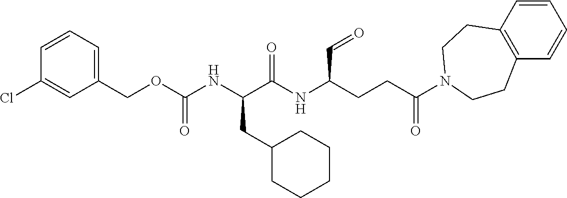 [SMILES: O=C[C@@H](CCC(=O)N1CCc2ccccc2CC1)NC(=O)[C@@H](CC1CCCCC1)NC(=O)OCc1cccc(Cl)c1] |
| A119 | 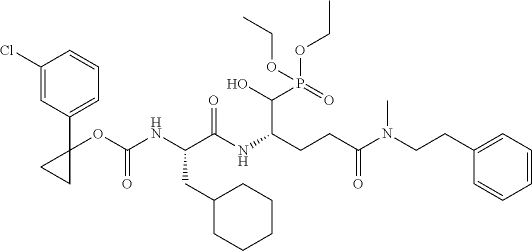 [SMILES: CCOP(=O)(OCC)C(O)[C@H](CCC(=O)N(C)CCc1ccccc1)NC(=O)[C@H](CC1CCCCC1)NC(=O)OC1(c2cccc(Cl)c2)CC1] |
| A120 | 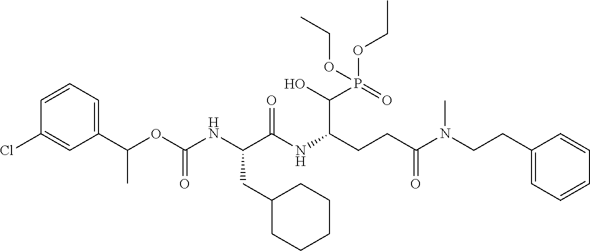 [SMILES: CCOP(=O)(OCC)C(O)[C@H](CCC(=O)N(C)CCc1ccccc1)NC(=O)[C@H](CC1CCCCC1)NC(=O)OC(C)c1cccc(Cl)c1] |
| A121 | 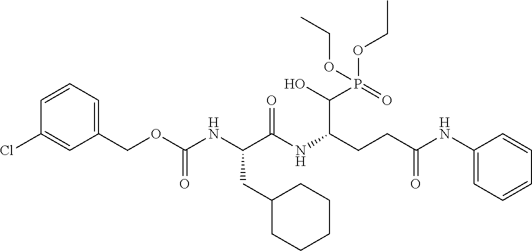 [SMILES: CCOP(=O)(OCC)C(O)[C@H](CCC(=O)Nc1ccccc1)NC(=O)[C@H](CC1CCCCC1)NC(=O)OCc1cccc(Cl)c1] |
| A122 | 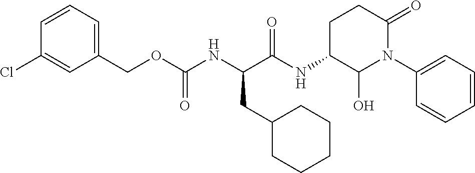 [SMILES: O=C(N[C@H](CC1CCCCC1)C(=O)N[C@@H]1CCC(=O)N(c2ccccc2)C1O)OCc1cccc(Cl)c1] |
| A123 | 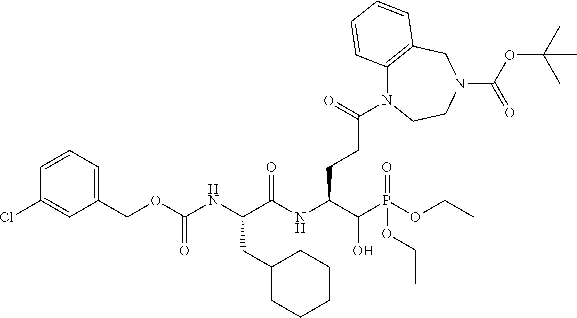 [SMILES: CCOP(=O)(OCC)C(O)[C@H](CCC(=O)N1CCN(C(=O)OC(C)(C)C)Cc2ccccc21)NC(=O)[C@H](CC1CCCCC1)NC(=O)OCc1cccc(Cl)c1] |
| A124 | 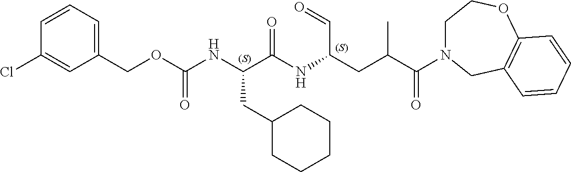 |
| A125 | 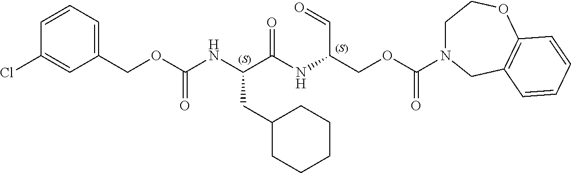 |
| A126 | 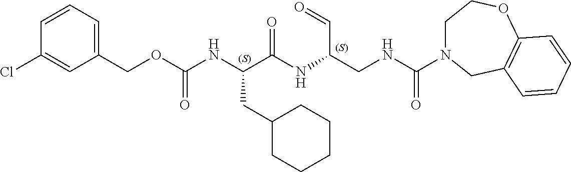 |
| A127 | 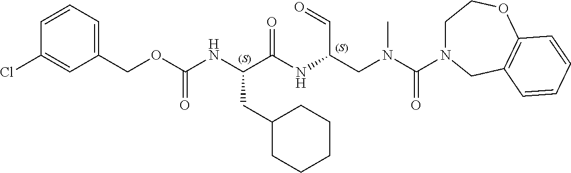 |
| A128 | 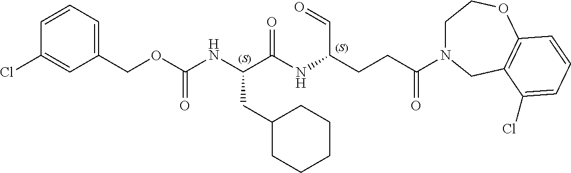 |
| A129 | 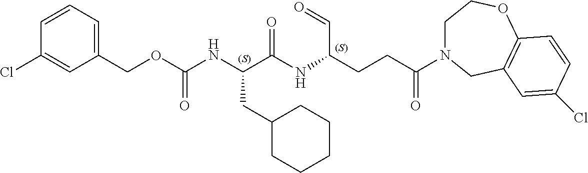 |
| A130 | 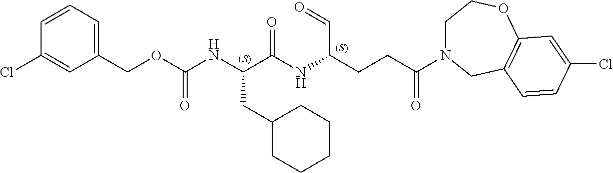 |
| A131 | 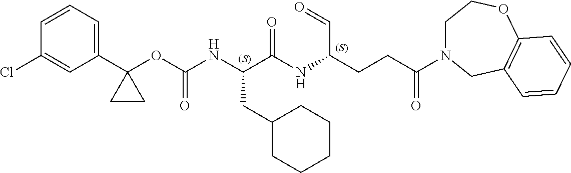 |
| A132 | 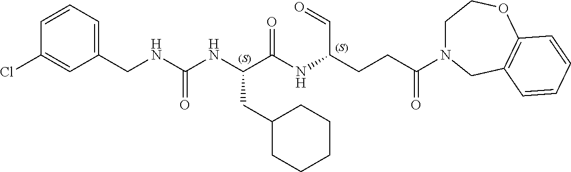 |
| A133 | 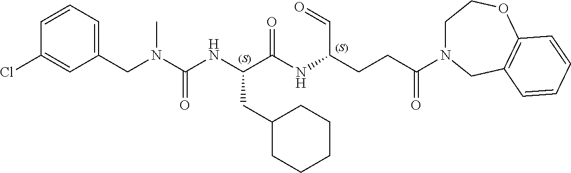 |
| A134 | 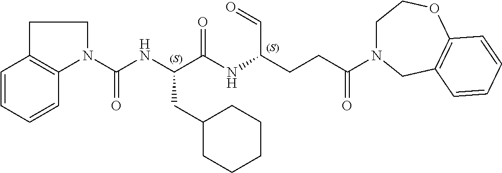 |
| A135 | 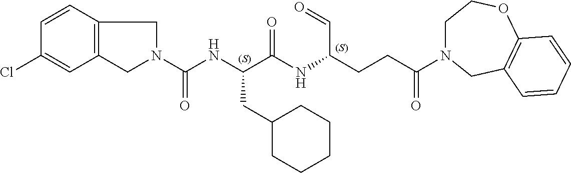 |
| A136 | 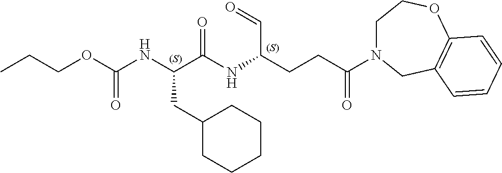 |
| A137 | 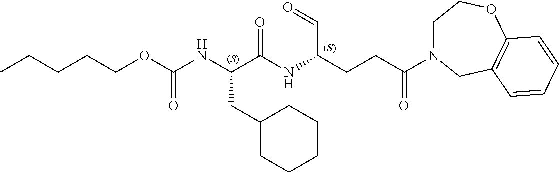 |
| A138 | 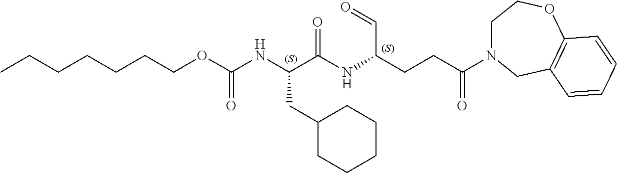 |
| A139 | 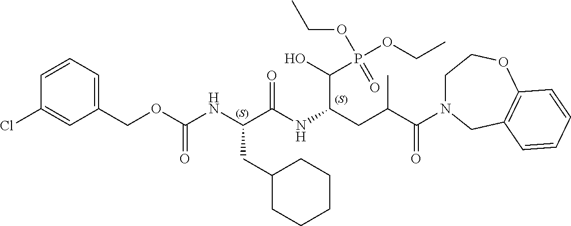 |
| A140 | 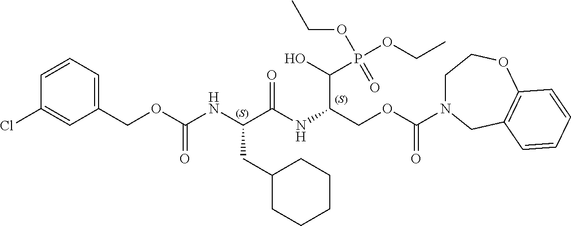 |
| A141 | 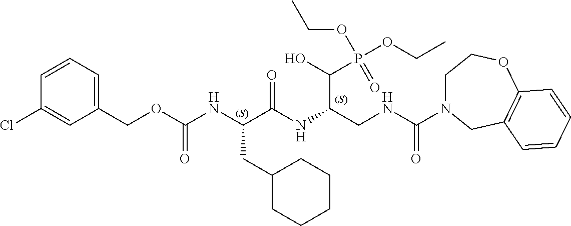 |
| A142 | 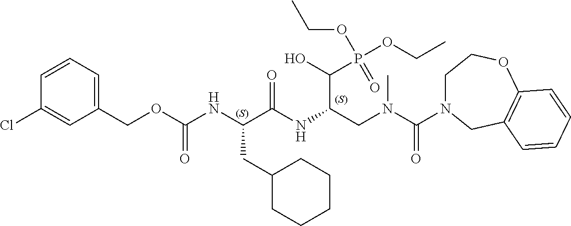 |
| A143 | 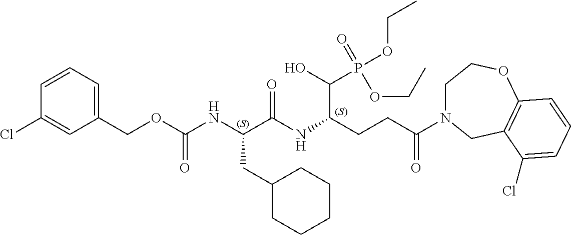 |
| A144 | 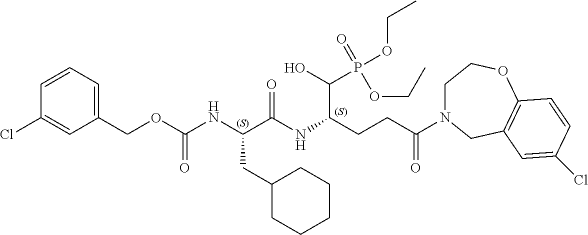 |
| A145 | 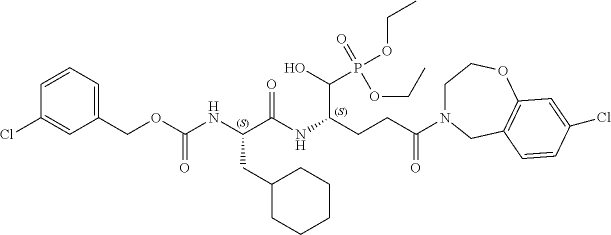 |
| A146 | 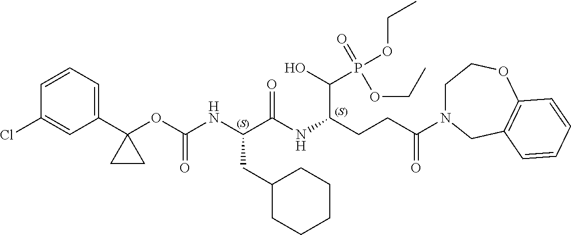 |
| A147 | 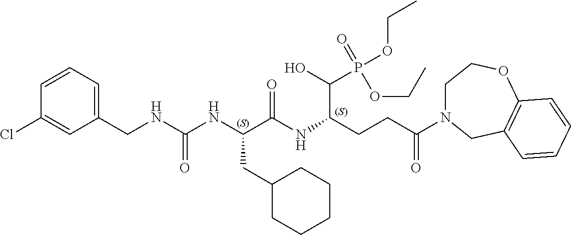 |
| A148 | 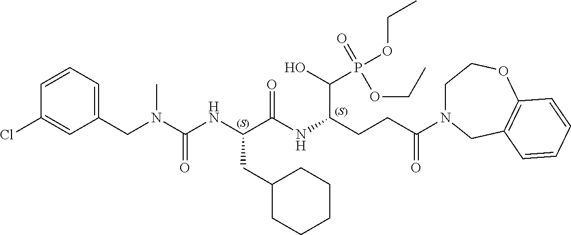 |
| A149 | 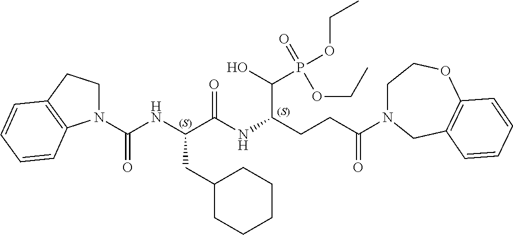 |
| A150 | 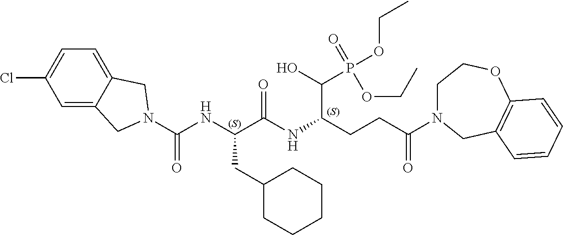 |
| A151 | 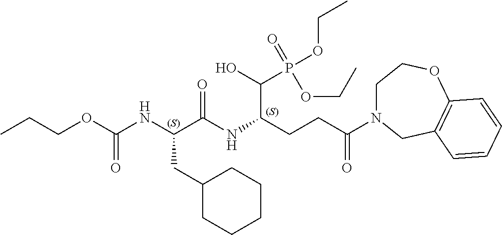 |
| A152 | 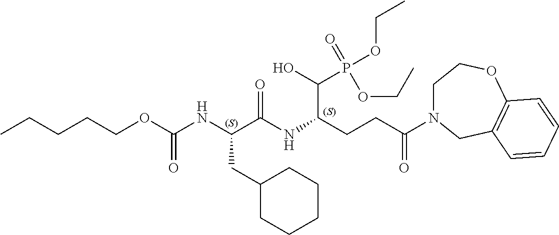 |
| A153 | 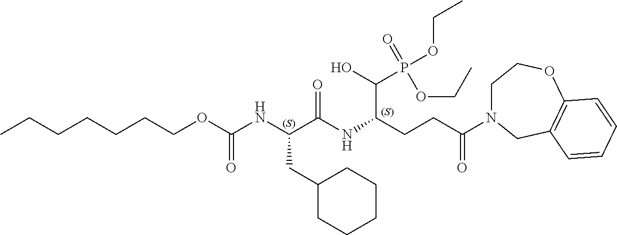 |
| A154 | 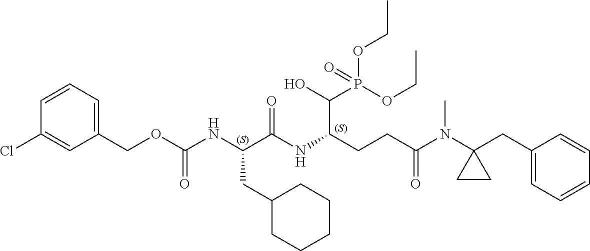 |
| A155 | 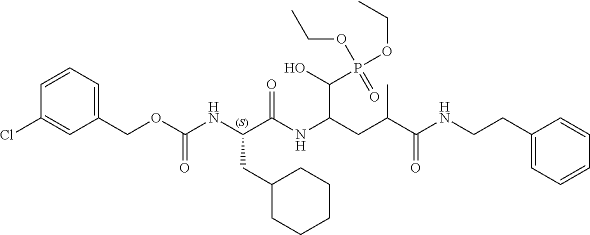 |
| A156 | 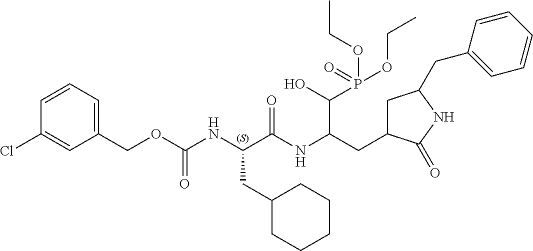 |
| A157 | 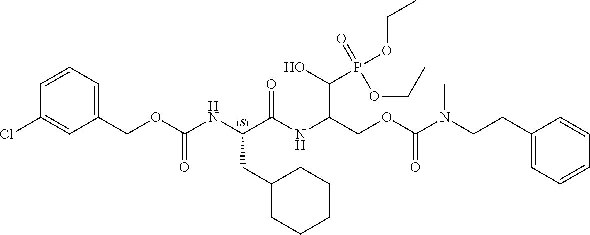 |
| A158 | 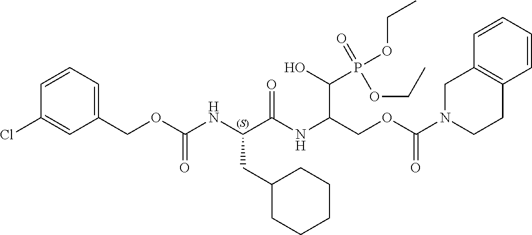 |
| A159 | 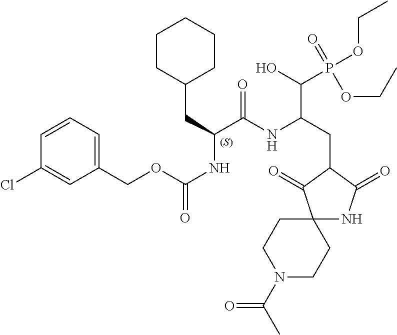 |
| A160 | 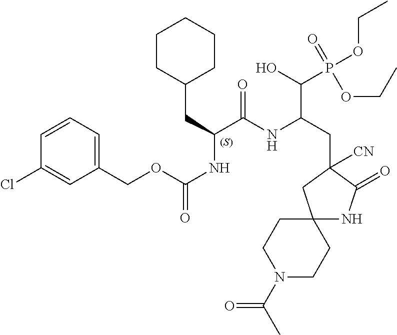 |
| A161 | 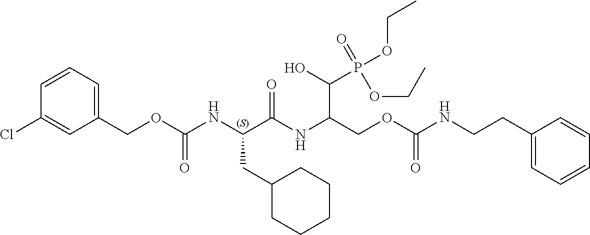 |
| A162 | 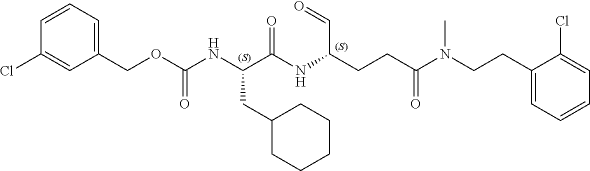 |
| A163 | 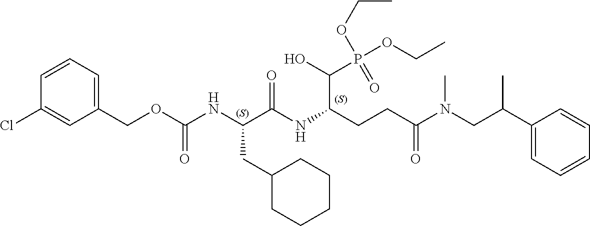 |
| A164 | 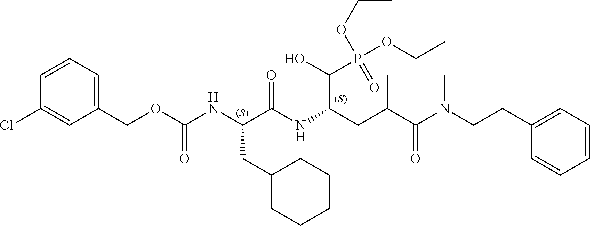 |
| A165 | 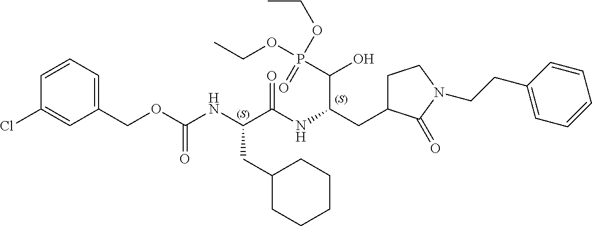 |
| A166 | 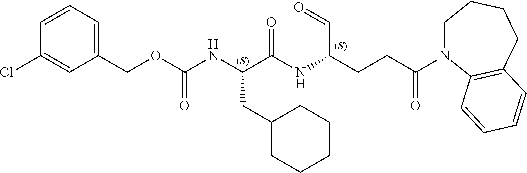 |
| A167 | 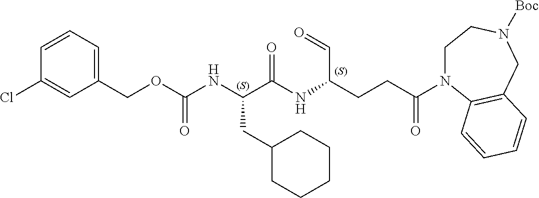 |
| A168 | 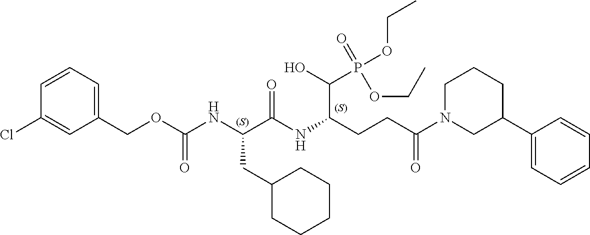 |
| A169 | 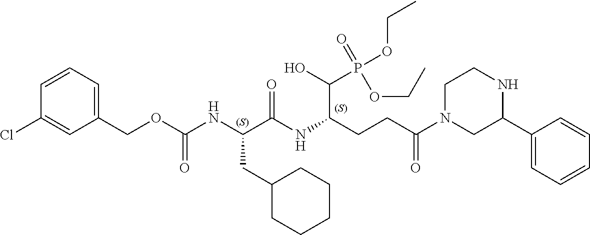 |
| A170 | 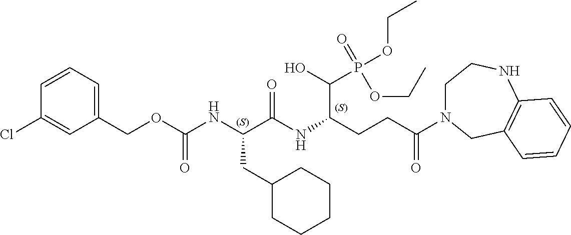 |
| A171 | 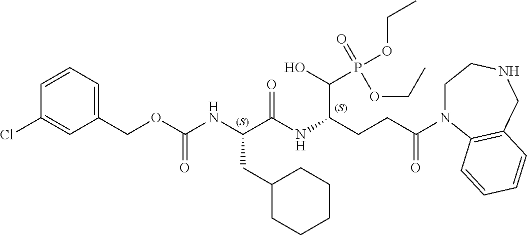 |
| A172 | 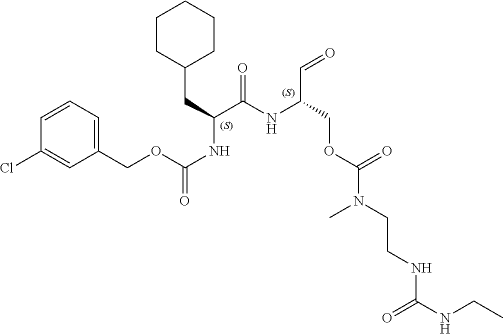 |
| A173 | 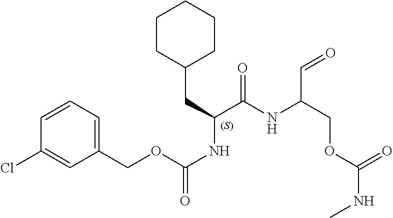 |
| A174 | 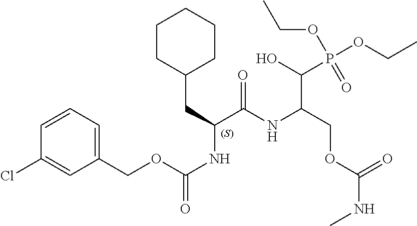 |
| A175 | 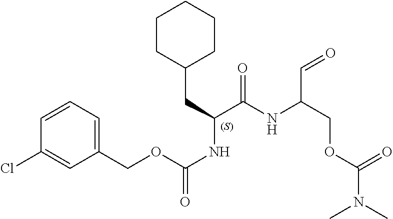 |
| A176 | 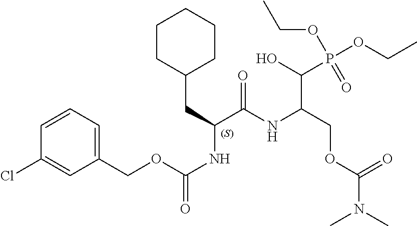 |
| A177 | 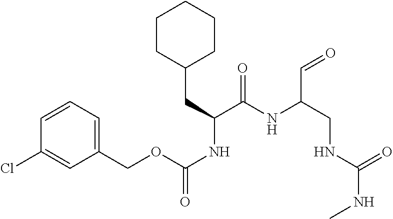 [SMILES: CNC(=O)NCC(C=O)NC(=O)[C@H](CC1CCCCC1)NC(=O)OCc1cccc(Cl)c1] |
| A178 | 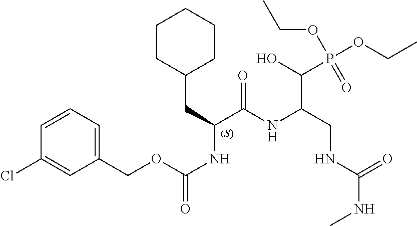 |
| A179 | 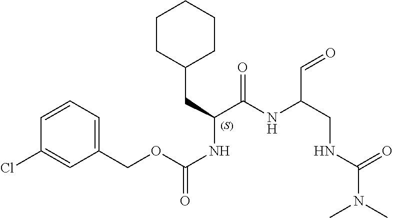 |
| A180 | 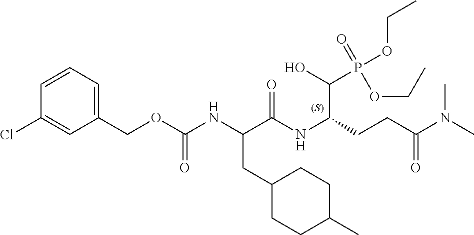 |
| A181 | 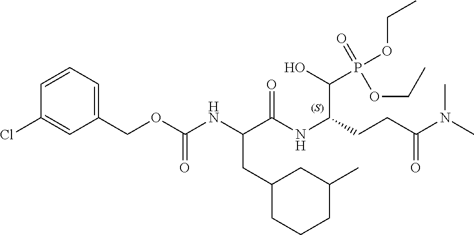 |
| A182 | 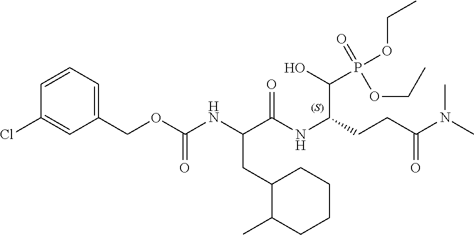 |
| A183 | 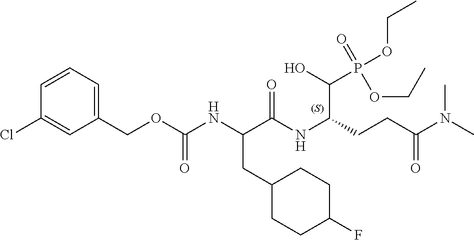 |
| A184 | 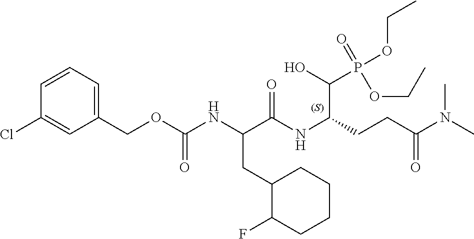 |
| A185 | 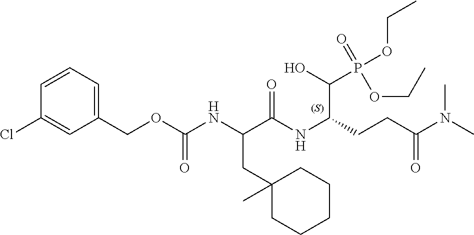 |
| A186 | 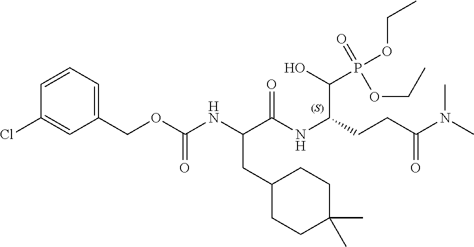 |
| A187 | 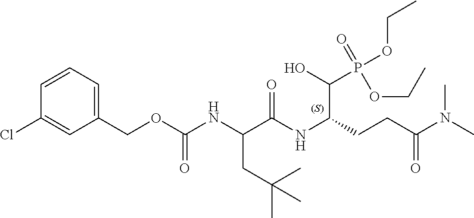 |
| A188 | 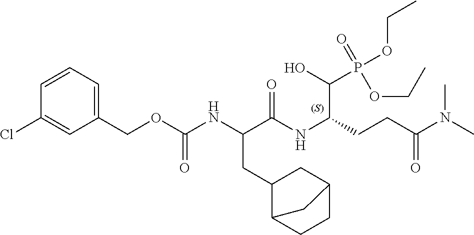 |
| A189 | 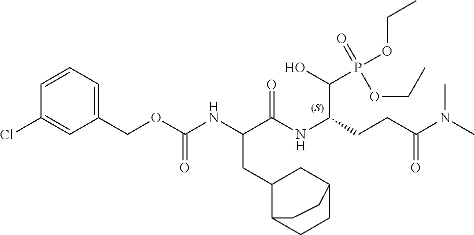 |
| A190 | 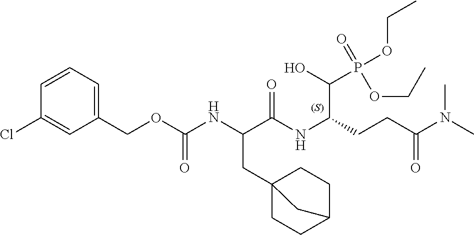 |
| A191 | 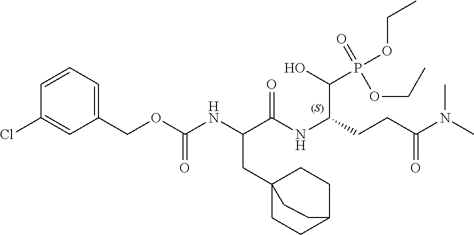 |
| A192 | 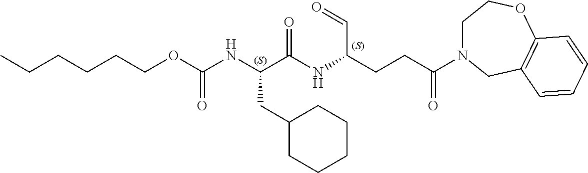 |
| A193 | 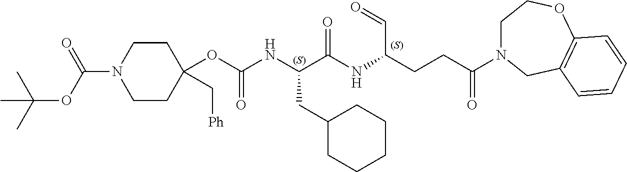 |
| A194 | 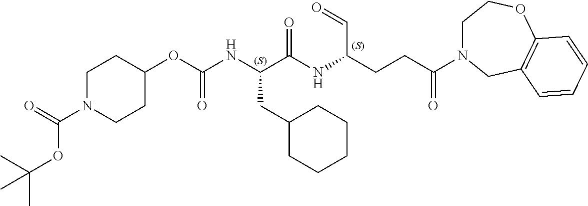 |
| A195 | 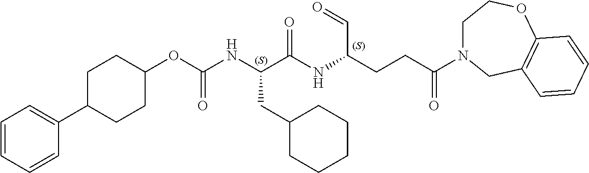 |
| A196 | 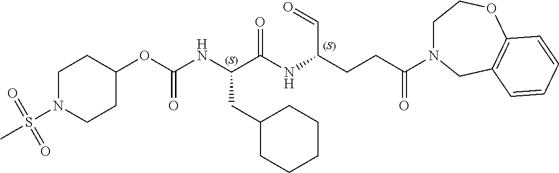 |
| A197 | 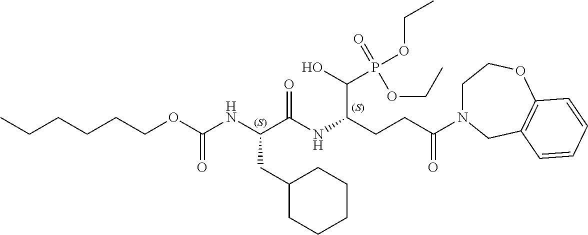 |
| A198 | 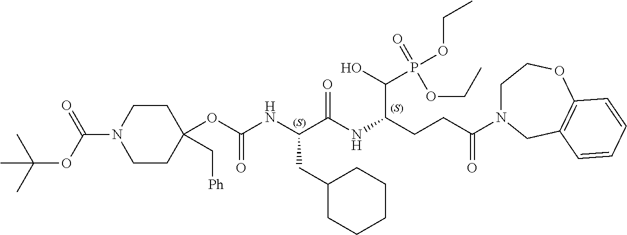 |
| A199 | 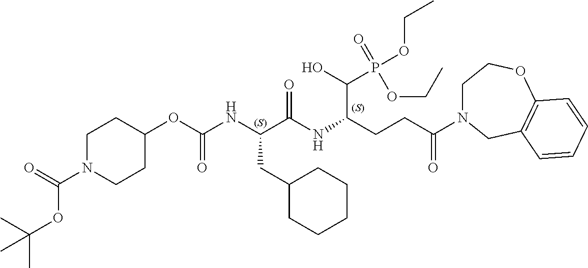 |
| A200 | 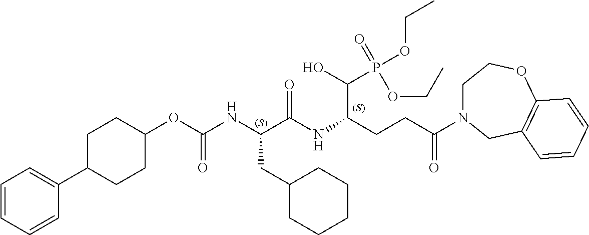 |
| A201 | 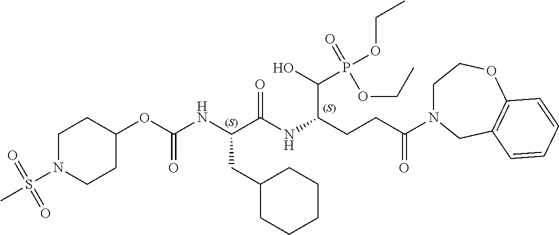 |
| A202 | 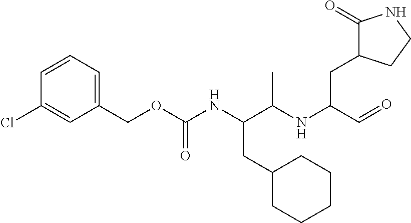 [SMILES: CC(NC(C=O)CC1CCNC1=O)C(CC1CCCCC1)NC(=O)OCc1cccc(Cl)c1] |
| A203 | 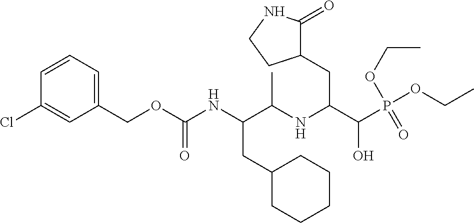 [SMILES: CCOP(=O)(OCC)C(O)C(CC1CCNC1=O)NC(C)C(CC1CCCCC1)NC(=O)OCc1cccc(Cl)c1] |
| A204 | 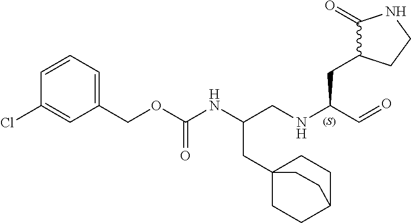 |
| A205 | 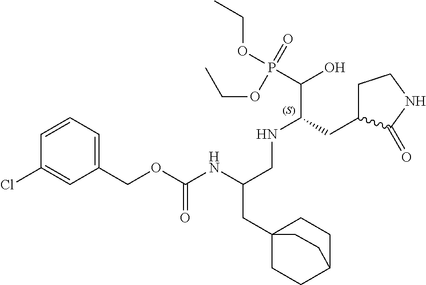 |
| A206 | 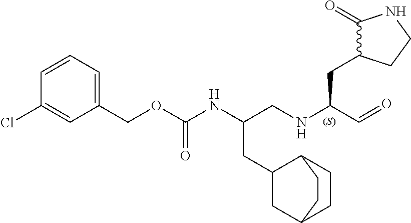 |
| A207 | 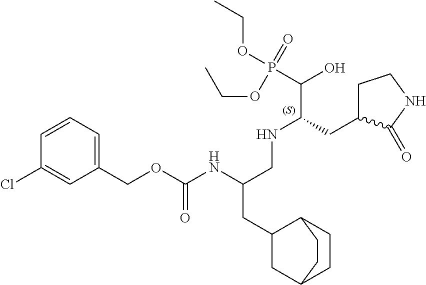 |
| A208 | 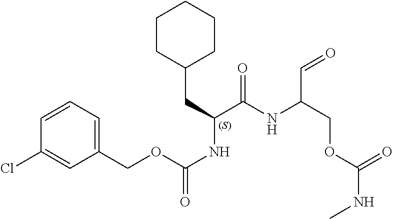 |
| A209 | 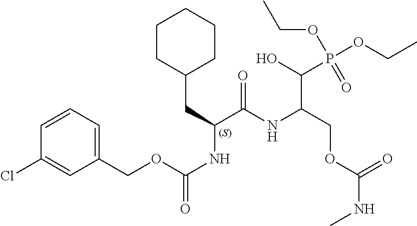 |
| A210 | 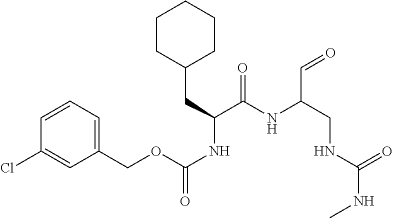 [SMILES: CNC(=O)NCC(C=O)NC(=O)[C@H](CC1CCCCC1)NC(=O)OCc1cccc(Cl)c1] |
| A211 | 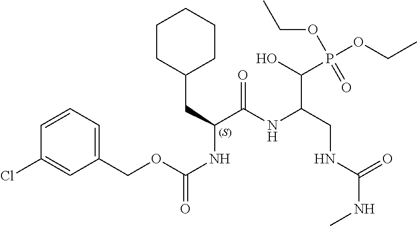 |
| A212 | 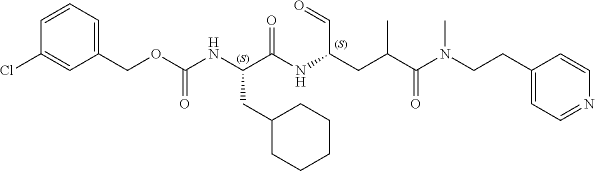 |
| A213 | 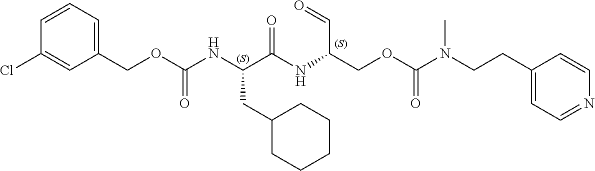 |
| A214 | 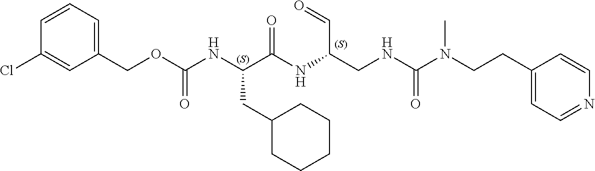 |
| A215 | 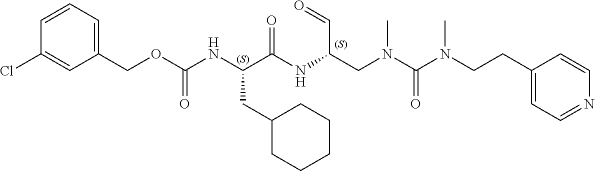 |
| A216 | 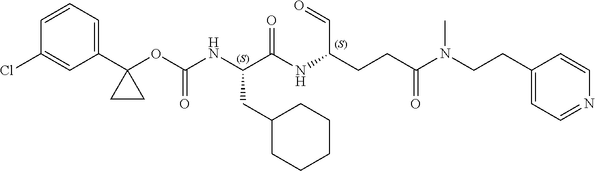 |
| A217 | 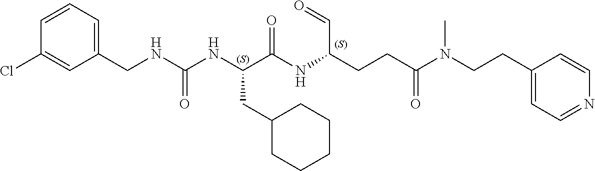 |
| A218 | 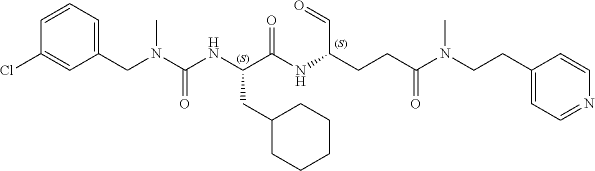 |
| A219 | 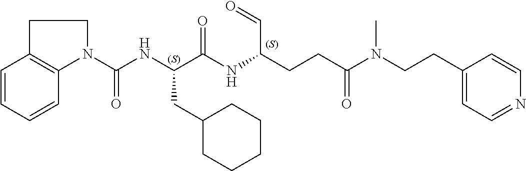 |
| A220 | 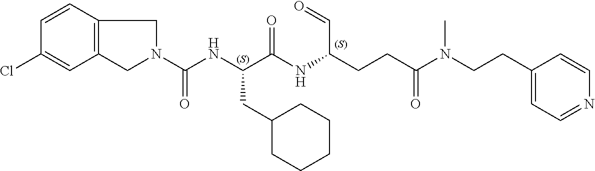 |
| A221 | 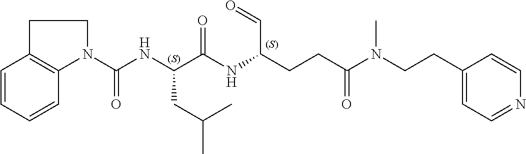 |
| A222 | 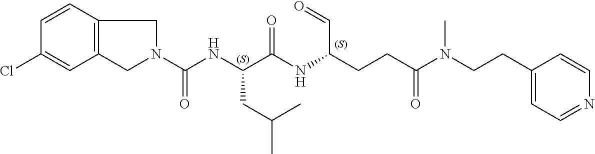 |
| A223 | 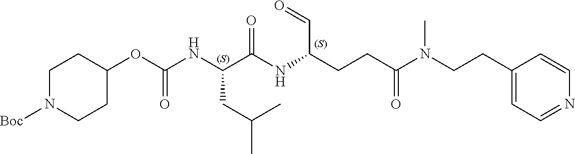 |
| A224 | 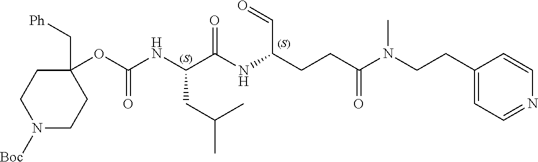 |
| A225 | 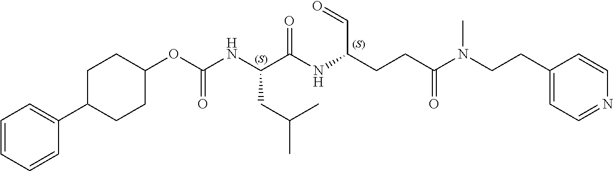 |
| A226 | 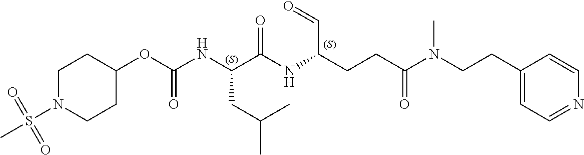 |
| A227 | 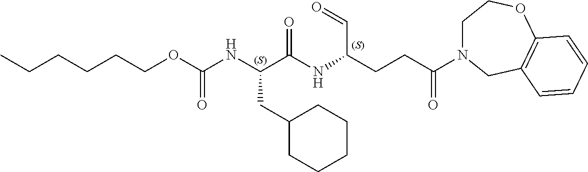 |
| A228 | 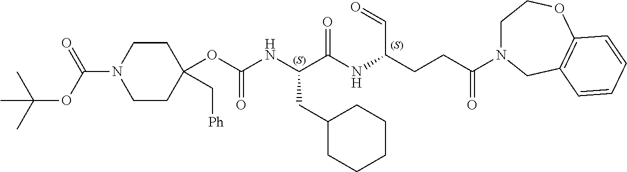 |
| A229 | 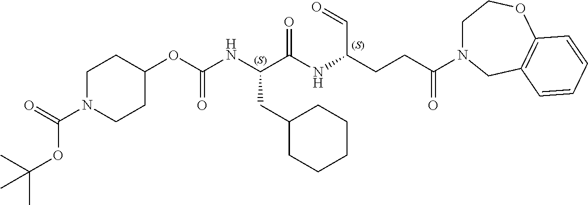 |
| A230 | 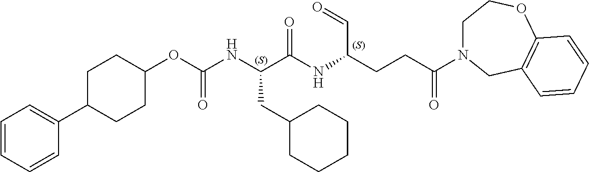 |
| A231 | 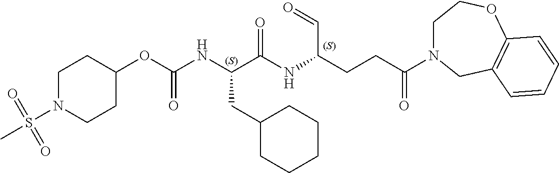 |
| A232 | 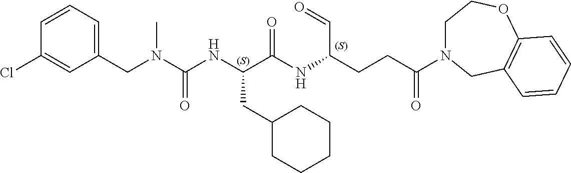 |
| A233 | 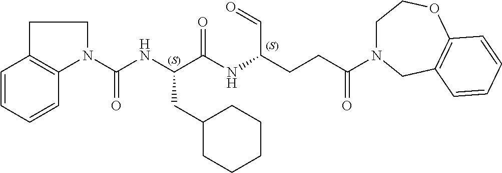 |
| A234 | 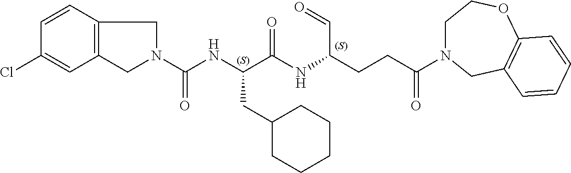 |
| A235 | 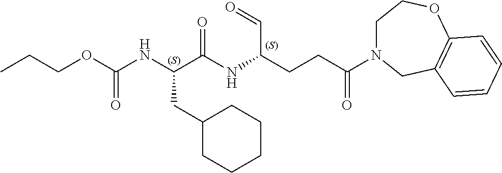 |
| A236 | 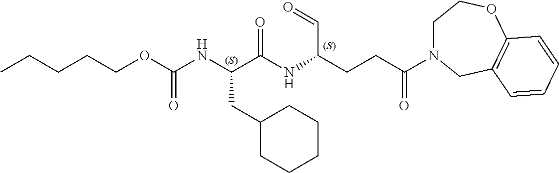 |
| A237 | 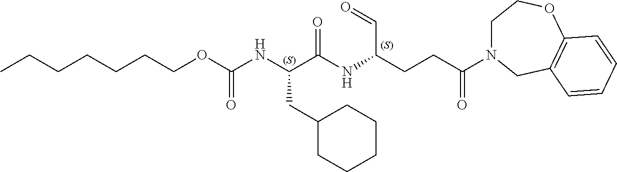 |

The compound can be a compound as listed in Table B, or a pharmaceutically acceptable salt thereof.

TABLE B

| Com- | |
|---|---|
| pound | |
| no. | Structure |
| B1 | 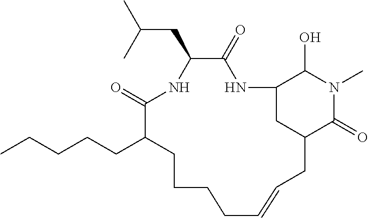 [SMILES: CCCCCC1CCCC/C=C/CC2CC(NC(=O)[C@H](CC(C)C)NC1=O)C(O)N(C)C2=O] |
| B4 | 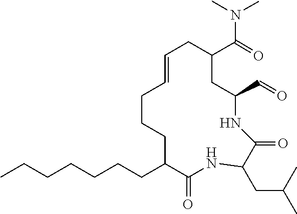 [SMILES: CCCCCCCC1CCC/C=C/CC(C(=O)N(C)C)C[C@@H](C=O)NC(=O)C(CC(C)C)NC1=O] |
| B7 | 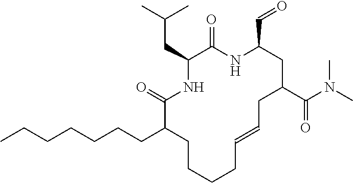 [SMILES: CCCCCCCC1CCCC/C=C/CC(C(=O)N(C)C)C[C@H](C=O)NC(=O)[C@H](CC(C)C)NC1=O] |
| B8 | 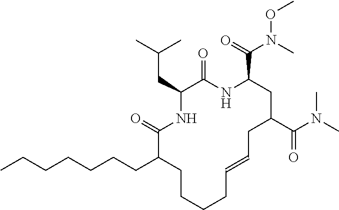 [SMILES: CCCCCCCC1CCCC/C=C/CC(C(=O)N(C)C)C[C@H](C(=O)N(C)OC)NC(=O)[C@H](CC(C)C)NC1=O] |
| B9 | 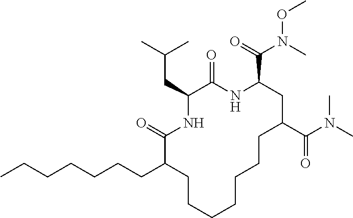 [SMILES: CCCCCCCC1CCCCCCCC(C(=O)N(C)C)C[C@H](C(=O)N(C)OC)NC(=O)[C@H](CC(C)C)NC1=O] |
| B10 | 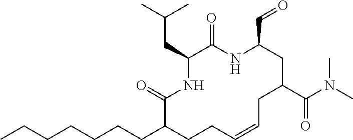 [SMILES: CCCCCCCC1CC/C=C\CC(C(=O)N(C)C)C[C@H](C=O)NC(=O)[C@H](CC(C)C)NC1=O] |
| B11 | 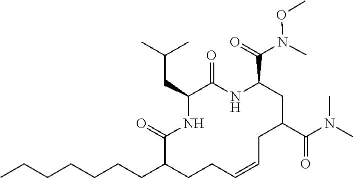 [SMILES: CCCCCCCC1CC/C=C\CC(C(=O)N(C)C)C[C@H](C(=O)N(C)OC)NC(=O)[C@H](CC(C)C)NC1=O] |
| B12 | 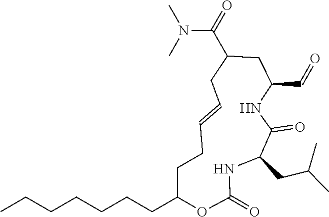 [SMILES: CCCCCCCC1CC/C=C/CC(C(=O)N(C)C)C[C@@H](C=O)NC(=O)[C@@H](CC(C)C)NC(=O)O1] |
| B13 | 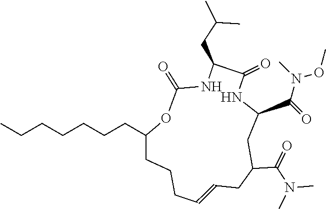 [SMILES: CCCCCCCC1CCC/C=C/CC(C(=O)N(C)C)C[C@H](C(=O)N(C)OC)NC(=O)[C@H](CC(C)C)NC(=O)O1] |
| B14 | 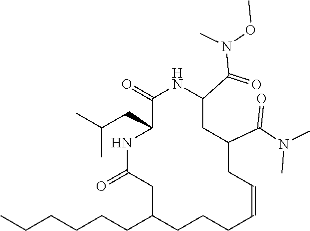 [SMILES: CCCCCCC1CCC/C=C\CC(C(=O)N(C)C)CC(C(=O)N(C)OC)NC(=O)[C@@H](CC(C)C)NC(=O)C1] |
| B15 | 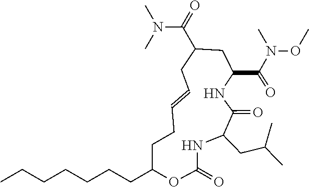 [SMILES: CCCCCCCC1CC/C=C/CC(C(=O)N(C)C)C[C@@H](C(=O)N(C)OC)NC(=O)C(CC(C)C)NC(=O)O1] |
| B16 | 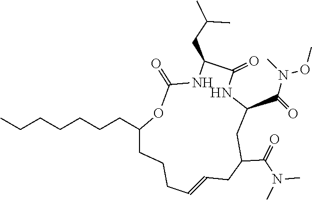 [SMILES: CCCCCCCC1CCC/C=C/CC(C(=O)N(C)C)C[C@H](C(=O)N(C)OC)NC(=O)[C@H](CC(C)C)NC(=O)O1] |
| B17 | 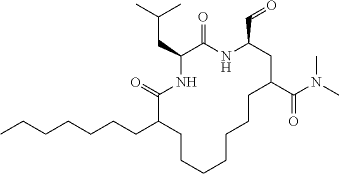 [SMILES: CCCCCCCC1CCCCCCCC(C(=O)N(C)C)C[C@H](C=O)NC(=O)[C@H](CC(C)C)NC1=O] |
| B18 | 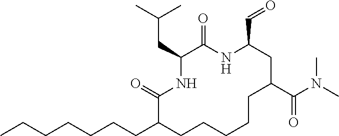 [SMILES: CCCCCCCC1CCCCCC(C(=O)N(C)C)C[C@H](C=O)NC(=O)[C@H](CC(C)C)NC1=O] |
| B19 | 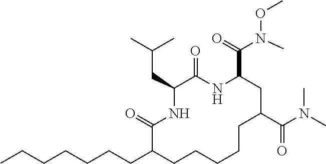 [SMILES: CCCCCCCC1CCCCCC(C(=O)N(C)C)C[C@H](C(=O)N(C)OC)NC(=O)[C@H](CC(C)C)NC1=O] |
| B20 | 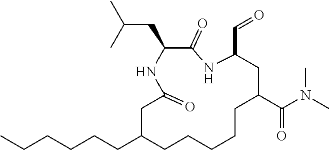 [SMILES: CCCCCCC1CCCCCC(C(=O)N(C)C)C[C@H](C=O)NC(=O)[C@H](CC(C)C)NC(=O)C1] |
| B21 | 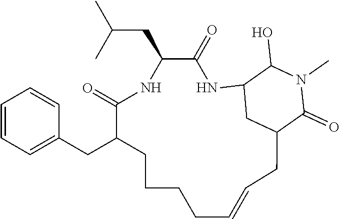 [SMILES: CC(C)C[C@@H]1NC(=O)C(Cc2ccccc2)CCCC/C=C/CC2CC(NC1=O)C(O)N(C)C2=O] |
| B22 | 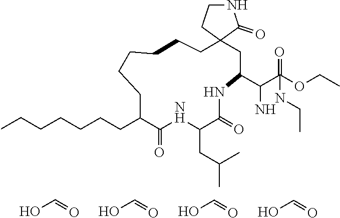 |
| B23 | 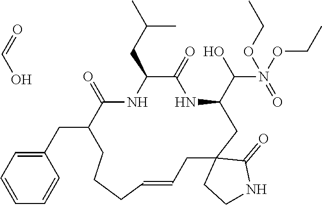 [SMILES: CCO[N+]([O-])(OCC)C(O)[C@H]1CC2(C/C=C/CCCC(Cc3ccccc3)C(=O)N[C@@H](CC(C)C)C(=O)N1)CCNC2=O ; O=CO] |
| B24 | 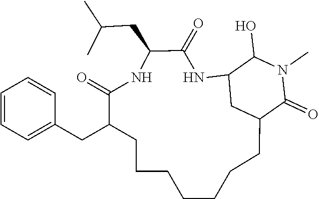 [SMILES: CC(C)C[C@@H]1NC(=O)C(Cc2ccccc2)CCCCCCCC2CC(NC1=O)C(O)N(C)C2=O] |
| B25 | 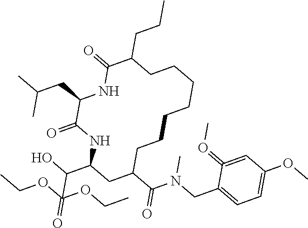 |
| B26 | 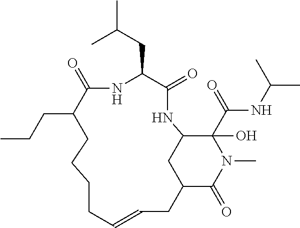 [SMILES: CCCC1CCCC/C=C/CC2CC(NC(=O)[C@H](CC(C)C)NC1=O)C(O)(C(=O)NC(C)C)N(C)C2=O] |
| B27 | 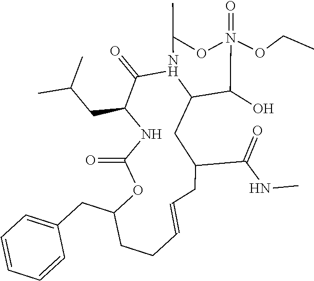 |
| B28 | 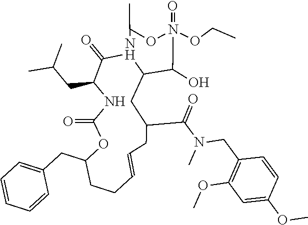 |
| B29 | 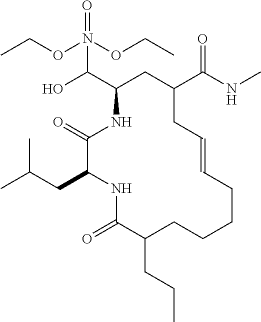 |
| B30 | 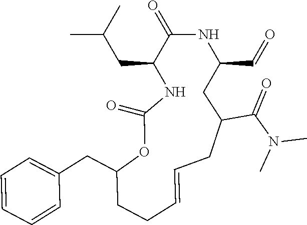 [SMILES: CC(C)C[C@@H]1NC(=O)OC(Cc2ccccc2)CC/C=C/CC(C(=O)N(C)C)C[C@H](C=O)NC1=O] |
| B31 | 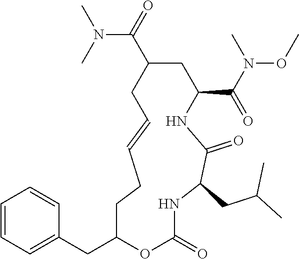 [SMILES: CON(C)C(=O)[C@@H]1CC(C(=O)N(C)C)C/C=C/CCC(Cc2ccccc2)OC(=O)N[C@H](CC(C)C)C(=O)N1] |
| B32 | 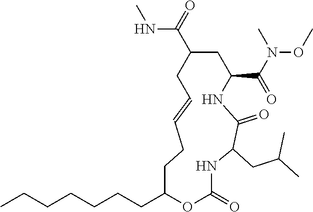 [SMILES: CCCCCCCC1CC/C=C/CC(C(=O)NC)C[C@@H](C(=O)N(C)OC)NC(=O)C(CC(C)C)NC(=O)O1] |
| B33 | 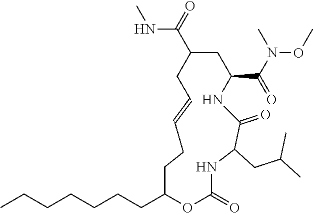 [SMILES: CCCCCCCC1CC/C=C/CC(C(=O)NC)C[C@@H](C(=O)N(C)OC)NC(=O)C(CC(C)C)NC(=O)O1] |
| B34 | 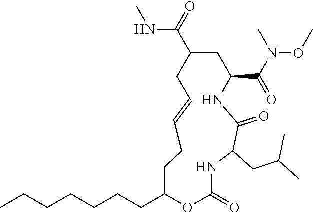 [SMILES: CCCCCCCC1CC/C=C/CC(C(=O)NC)C[C@@H](C(=O)N(C)OC)NC(=O)C(CC(C)C)NC(=O)O1] |
| B35 | 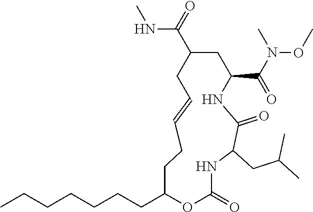 [SMILES: CCCCCCCC1CC/C=C/CC(C(=O)NC)C[C@@H](C(=O)N(C)OC)NC(=O)C(CC(C)C)NC(=O)O1] |
| B36 | 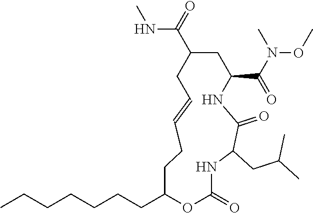 [SMILES: CCCCCCCC1CC/C=C/CC(C(=O)NC)C[C@@H](C(=O)N(C)OC)NC(=O)C(CC(C)C)NC(=O)O1] |
| B37 | 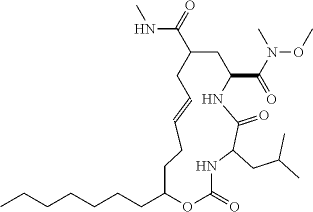 [SMILES: CCCCCCCC1CC/C=C/CC(C(=O)NC)C[C@@H](C(=O)N(C)OC)NC(=O)C(CC(C)C)NC(=O)O1] |
| B38 | 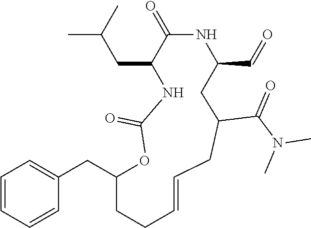 |
| B41 | 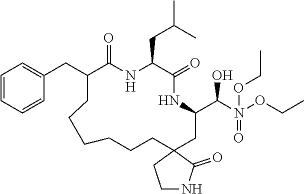 [SMILES: CCO[N+]([O-])(OCC)[C@H](O)[C@H]1CC2(CCCCCCC(Cc3ccccc3)C(=O)N[C@@H](CC(C)C)C(=O)N1)CCNC2=O] |
| B42 | 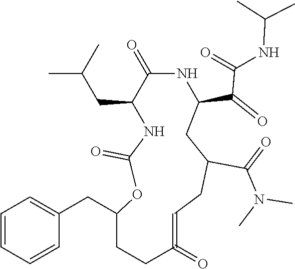 |
| B43 | 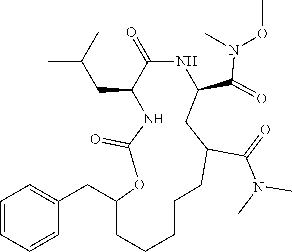 [SMILES: CON(C)C(=O)[C@H]1CC(C(=O)N(C)C)CCCCCC(Cc2ccccc2)OC(=O)N[C@@H](CC(C)C)C(=O)N1] |
| B44 | 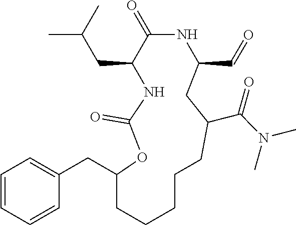 [SMILES: CC(C)C[C@@H]1NC(=O)OC(Cc2ccccc2)CCCCCC(C(=O)N(C)C)C[C@H](C=O)NC1=O] |
| B45 | 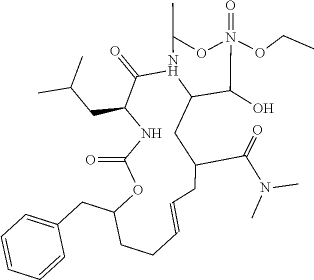 |
| B46 | 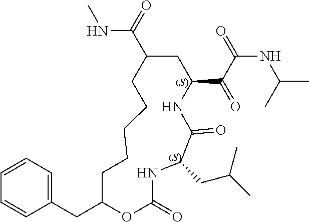 |
| B47 | 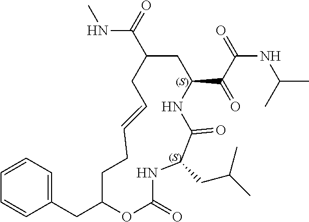 |
| B48 | 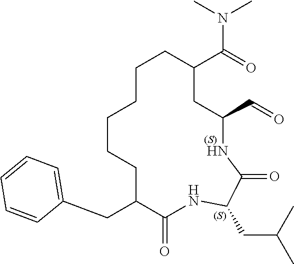 |
| B49 | 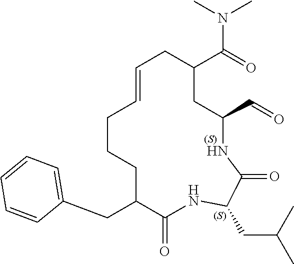 |
| B50 | 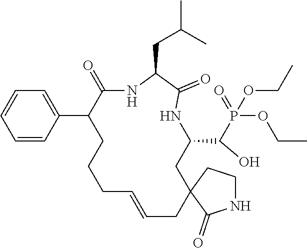 [SMILES: CCOP(=O)(OCC)C(O)[C@@H]1CC2(C/C=C/CCCC(c3ccccc3)C(=O)N[C@@H](CC(C)C)C(=O)N1)CCNC2=O] |
| B51 | 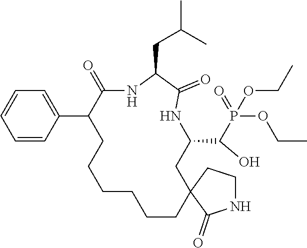 [SMILES: CCOP(=O)(OCC)C(O)[C@@H]1CC2(CCCCCCC(c3ccccc3)C(=O)N[C@@H](CC(C)C)C(=O)N1)CCNC2=O] |
| B52 | 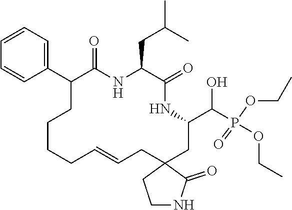 [SMILES: CCOP(=O)(OCC)C(O)[C@@H]1CC2(C/C=C/CCCCC(c3ccccc3)C(=O)N[C@@H](CC(C)C)C(=O)N1)CCNC2=O] |
| B53 | 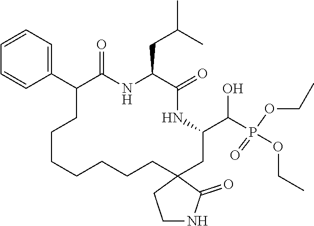 [SMILES: CCOP(=O)(OCC)C(O)[C@@H]1CC2(CCCCCCCC(c3ccccc3)C(=O)N[C@@H](CC(C)C)C(=O)N1)CCNC2=O] |
| B54 | 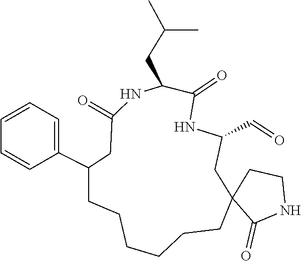 [SMILES: CC(C)C[C@@H]1NC(=O)CC(c2ccccc2)CCCCCCC2(CCNC2=O)C[C@@H](C=O)NC1=O] |
| B55 | 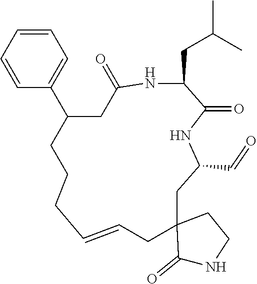 [SMILES: CC(C)C[C@@H]1NC(=O)CC(c2ccccc2)CCC/C=C/CC2(CCNC2=O)C[C@@H](C=O)NC1=O] |
| B56 | 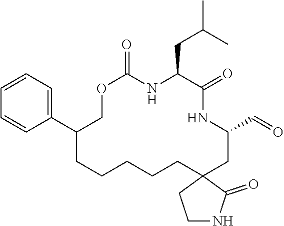 [SMILES: CC(C)C[C@@H]1NC(=O)OCC(c2ccccc2)CCCCCC2(CCNC2=O)C[C@@H](C=O)NC1=O] |
| B57 | 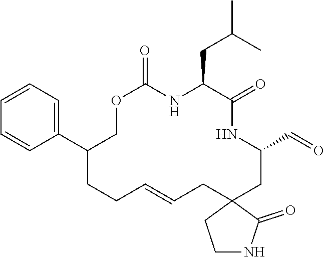 [SMILES: CC(C)C[C@@H]1NC(=O)OCC(c2ccccc2)CC/C=C/CC2(CCNC2=O)C[C@@H](C=O)NC1=O] |
| B58 | 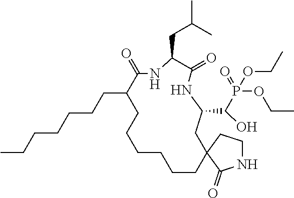 [SMILES: CCCCCCCC1CCCCCCC2(CCNC2=O)C[C@@H](C(O)P(=O)(OCC)OCC)NC(=O)[C@H](CC(C)C)NC1=O] |
| B59 | 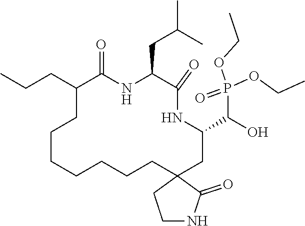 [SMILES: CCCC1CCCCCCCC2(CCNC2=O)C[C@@H](C(O)P(=O)(OCC)OCC)NC(=O)[C@H](CC(C)C)NC1=O] |
| B60 | 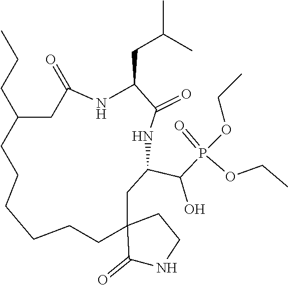 [SMILES: CCCC1CCCCCCC2(CCNC2=O)C[C@@H](C(O)P(=O)(OCC)OCC)NC(=O)[C@H](CC(C)C)NC(=O)C1] |
| B61 | 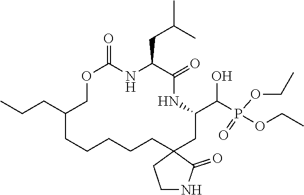 [SMILES: CCCC1CCCCCC2(CCNC2=O)C[C@@H](C(O)P(=O)(OCC)OCC)NC(=O)[C@H](CC(C)C)NC(=O)OC1] |
| B62 | 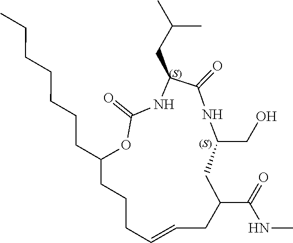 |
| B63 | 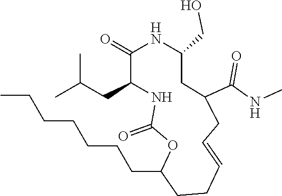 [SMILES: CCCCCCCC1CC/C=C/CC(C(=O)NC)C[C@@H](CO)NC(=O)[C@H](CC(C)C)NC(=O)O1] |
| B64 | 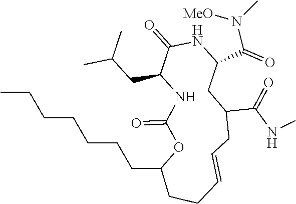 [SMILES: CCCCCCCC1CC/C=C/CC(C(=O)NC)C[C@@H](C(=O)N(C)OC)NC(=O)[C@H](CC(C)C)NC(=O)O1] |
| B65 | 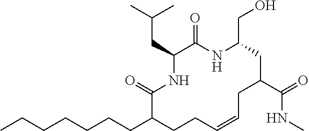 [SMILES: CCCCCCCC1CC/C=C\CC(C(=O)NC)C[C@@H](CO)NC(=O)[C@H](CC(C)C)NC1=O] |
| B66 | 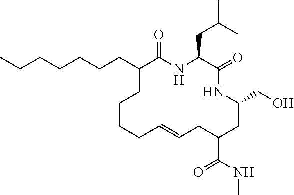 [SMILES: CCCCCCCC1CCCC/C=C/CC(C(=O)NC)C[C@@H](CO)NC(=O)[C@H](CC(C)C)NC1=O] |
| B67 | 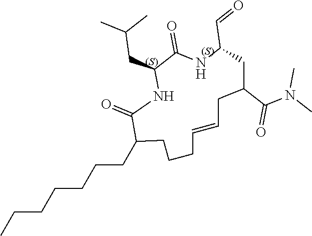 |
| B68 | 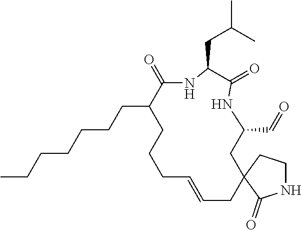 [SMILES: CCCCCCCC1CCC/C=C/CC2(CCNC2=O)C[C@@H](C=O)NC(=O)[C@H](CC(C)C)NC1=O] |
| B69 | 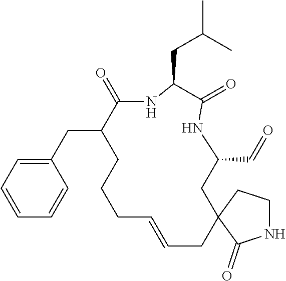 [SMILES: CC(C)C[C@@H]1NC(=O)C(Cc2ccccc2)CCC/C=C/CC2(CCNC2=O)C[C@@H](C=O)NC1=O] |
| B70 | 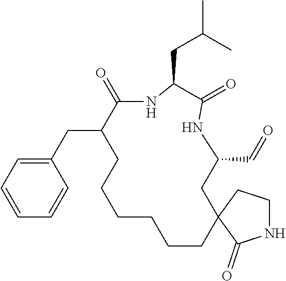 [SMILES: CC(C)C[C@@H]1NC(=O)C(Cc2ccccc2)CCCCCCC2(CCNC2=O)C[C@@H](C=O)NC1=O] |
| B71 | 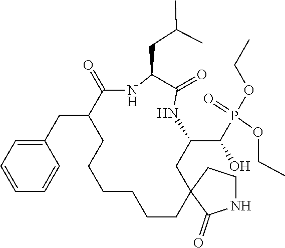 [SMILES: CCOP(=O)(OCC)[C@H](O)[C@@H]1CC2(CCCCCCC(Cc3ccccc3)C(=O)N[C@@H](CC(C)C)C(=O)N1)CCNC2=O] |

In some cases, the compound is selected from A6, A8, A10, A15, A18, A27, A57, B22, and B37, or a pharmaceutically acceptable salt thereof.

The compounds disclosed herein can be useful as inhibitors of norovirus or coronavirus replication in biological samples or in a patient. These compounds can also be useful in reducing the amount of noroviruses or coronaviruses (viral titer) in a biological sample or in a patient. They can also be useful for therapeutic and prophylactic treatment of infections caused by the noroviruses or coronaviruses in a biological sample or in a patient.

### Pharmaceutically Acceptable Salts

The compounds described herein can exist in free form, or, where appropriate, as salts. Those salts that are pharmaceutically acceptable are of particular interest since they are useful in administering the compounds described below for medical purposes. Salts that are not pharmaceutically acceptable are useful in manufacturing processes, for isolation and purification purposes, and in some instances, for use in separating stereoisomeric forms of the compounds of the disclosure or intermediates thereof.

As used herein, the term “pharmaceutically acceptable salt” refers to salts of a compound which are, within the scope of sound medical judgment, suitable for use in contact with the tissues of humans and lower animals without undue side effects, such as, toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds described herein include those derived from suitable inorganic and organic acids and bases. These salts can be prepared in situ during the final isolation and purification of the compounds.

Where the compound described herein contains a basic group, or a sufficiently basic bioisostere, acid addition salts can be prepared by 1) reacting the purified compound in its free-base form with a suitable organic or inorganic acid and 2) isolating the salt thus formed. In practice, acid addition salts might be a more convenient form for use and use of the salt amounts to use of the free basic form.

Examples of pharmaceutically acceptable, non-toxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Where the compound described herein contains a carboxy group or a sufficiently acidic bioisostere, base addition salts can be prepared by 1) reacting the purified compound in its acid form with a suitable organic or inorganic base and 2) isolating the salt thus formed. In practice, use of the base addition salt might be more convenient and use of the salt form inherently amounts to use of the free acid form. Salts derived from appropriate bases include alkali metal (e.g., sodium, lithium, and potassium), alkaline earth metal (e.g., magnesium and calcium), ammonium and N⁺(C₁₋₄ alkyl)₄ salts. This disclosure also envisions the quaternization of any basic nitrogen-containing groups of the compounds disclosed herein. Water or oil-soluble or dispersible products may be obtained by such quaternization.

Basic addition salts include pharmaceutically acceptable metal and amine salts. Suitable metal salts include the sodium, potassium, calcium, barium, zinc, magnesium, and aluminum. The sodium and potassium salts are usually preferred. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which are frequently used in medicinal chemistry because of their low toxicity and acceptability for medical use. Ammonia, ethylenediamine, N-methyl-glucamine, lysine, arginine, ornithine, choline, N,N′-dibenzylethylenediamine, chloroprocaine, dietanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, dicyclohexylamine and the like.

Other acids and bases, while not in themselves pharmaceutically acceptable, may be employed in the preparation of salts useful as intermediates in obtaining the compounds described herein and their pharmaceutically acceptable acid or base addition salts.

It should be understood that a compound disclosed herein can be present as a mixture/combination of different pharmaceutically acceptable salts. Also contemplated are mixtures/combinations of compounds in free form and pharmaceutically acceptable salts.

### II. Pharmaceutical Compositions

The compounds described herein can be formulated into pharmaceutical compositions that further comprise a pharmaceutically acceptable carrier, diluent, adjuvant or vehicle. In embodiments, the present disclosure relates to a pharmaceutical composition comprising a compound described above or salt thereof, and a pharmaceutically acceptable carrier, diluent, adjuvant or vehicle. In embodiments, the pharmaceutical composition comprises a safe and effective amount of a compound as disclosed herein or a pharmaceutically acceptable salt thereof and a pharmaceutically acceptable carrier, diluent, adjuvant or vehicle. Pharmaceutically acceptable carriers include, for example, pharmaceutical diluents, excipients or carriers suitably selected with respect to the intended form of administration, and consistent with conventional pharmaceutical practices.

An “effective amount” includes a “therapeutically effective amount” and a “prophylactically effective amount”. The term “therapeutically effective amount” refers to an amount effective in treating and/or ameliorating a norovirus or coronavirus virus infection in a patient. The term “prophylactically effective amount” refers to an amount effective in preventing and/or substantially lessening the chances or the size of norovirus or coronavirus virus infection outbreak.

A pharmaceutically acceptable carrier may contain inert ingredients which do not unduly inhibit the biological activity of the compounds. The pharmaceutically acceptable carriers should be biocompatible, e.g., non-toxic, non-inflammatory, non-immunogenic or devoid of other undesired reactions or side-effects upon the administration to a subject. Standard pharmaceutical formulation techniques can be employed.

The pharmaceutically acceptable carrier, adjuvant, or vehicle, as used herein, includes any solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds described herein, such as by producing any undesirable biological effect or otherwise interacting in a deleterious manner with any other component(s) of the pharmaceutically acceptable composition, its use is contemplated to be within the scope of this disclosure. As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful or uncomfortable or risky. Side effects include, but are not limited to fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances and sexual dysfunction.

Some examples of materials which can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

### Formulations for Pulmonary Delivery

In some embodiments, the pharmaceutical compositions disclosed herein are adapted to be administered to the lower respiratory tract (e.g., the lungs) directly through the airways by inhalation. Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose or starch. Inhalable dry powder compositions may be presented in capsules and cartridges of gelatin or a like material, or blisters of laminated aluminum foil for use in an inhaler or insufflators. Each capsule or cartridge may generally contain e.g., from about 10 mg to about 100 g of each active compound. Alternatively, the composition may be presented without excipients.

The inhalable compositions may be packaged for unit dose or multi-dose delivery. For example, the compositions can be packaged for multi-dose delivery in a manner analogous to that described in GB 2242134, U.S. Pat. Nos. 6,632,666, 5,860,419, 5,873,360, and 5,590,645 (all illustrating the “Diskus” device), or GB2i78965, GB2129691, GB2169265, U.S. Pat. Nos. 4,778,054, 4,811,731 and 5,035,237 (which illustrate the “Diskhaler” device), or EP 69715 (“Turbuhaler” device), or GB 2064336 and U.S. Pat. No. 4,353,656 (“Rotahaler” device).

Spray compositions for topical delivery to the lung by inhalation may be formulated as aqueous solutions or suspensions or as aerosols delivered from pressurized packs, such as a metered dose inhaler (MDI), with the use of a suitable liquefied propellant, including hydrofluoroalkanes such as dichlorodifluoromethane, trichlorofluoromethane, dichlorotetrafluoroethane, and especially 1,1,1,2-tetrafluoroethane, 1,1,1,2,3,3,3-heptafluoro-n-propane and mixtures thereof. Aerosol compositions suitable for inhalation can be presented either as suspensions or as solutions.

Medicaments for administration by inhalation typically have a controlled particle size. The optimum particle size for inhalation into the bronchial system is usually about 1 to about 10 μm, and in some embodiments, from about 2 to about 5 μm. Particles having a size above about 20 μm are generally too large when inhaled to reach the small airways. To achieve these particle sizes the particles of the active ingredient may be subjected to a size reducing process such as micronization. The desired size fraction may be separated out by air classification or sieving. Preferably, the particles will be crystalline.

Intranasal sprays may be formulated with aqueous or non-aqueous vehicles with the addition of agents such as thickening agents, buffer salts or acid or alkali to adjust the pH, isotonic adjusting agents or anti-oxidants.

Solutions for inhalation by nebulization may be formulated with an aqueous vehicle with the addition of agents such as acid or alkali, buffer salts, isotonic adjusting agents or antimicrobial agents. They may be sterilized by filtration or heating in an autoclave, or presented as a non-sterile product. Nebulizers supply the aerosol as a mist created from an aqueous formulation.

In some embodiments, the pharmaceutical compositions disclosed herein can be formulated with supplementary active ingredients.

In some embodiments, the pharmaceutical composition disclosed herein is administered from a dry powder inhaler. In other embodiments, the pharmaceutical composition disclosed herein is administered by an aerosol dispensing device, optionally in conjunction with an inhalation chamber such as the “Volumatic” ® inhalation chamber.

The carrier can be a solvent or dispersion medium containing, for example, water, ethanol, polyol (for example, glycerol, propylene glycol and liquid polyethylene glycol, and the like), suitable mixtures thereof, and/or vegetable oils. The proper fluidity can be maintained, for example, by the use of a coating such as, for example, lecithin, by the maintenance of the required particle size in the case of dispersion and by the use of surfactants. Preventing the action of microorganisms in the compositions disclosed herein is achieved by adding antibacterial and/or antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In some embodiments, a pharmaceutical composition can be within a matrix which controls the release of the composition. In some embodiments, the matrix can comprise lipid, polyvinyl alcohol, polyvinyl acetate, polycaprolactone, poly(glycolic)acid, poly(lactic)acid, polycaprolactone, polylactic acid, polyanhydrides, polylactide-co-glycolides, polyamino acids, polyethylene oxide, acrylic terminated polyethylene oxide, polyamides, polyethylenes, polyacrylonitriles, polyphosphazenes, poly(ortho esters), sucrose acetate isobutyrate (SAIB), and combinations thereof and other polymers such as those disclosed, for example, in U.S. Pat. Nos. 6,667,371; 6,613,355; 6,596,296; 6,413,536; 5,968,543; 4,079,038; 4,093,709; 4,131,648; 4,138,344; 4,180,646; 4,304,767; 4,946,931, each of which is expressly incorporated by reference herein in its entirety. In these embodiments, the matrix sustainedly releases the drug.

Pharmaceutically acceptable carriers and/or diluents may also include any solvents, dispersion media, coatings, antibacterials and/or antifungals, isotonic and absorption delaying agents and the like. The use of such media and agents for pharmaceutically active substances is well known in the art. Except insofar as any conventional medium or agent is incompatible with the active ingredient, use thereof in the pharmaceutical compositions is contemplated.

The pharmaceutical compositions can be formulated for administration in accordance with conventional techniques. See, e.g., Remington, The Science and Practice of Pharmacy (20th Ed. 2000). For example, the intranasal pharmaceutical compositions of the present disclosure can be formulated as an aerosol (this term includes both liquid and dry powder aerosols). Aerosols of liquid particles can be produced by any suitable means, such as with a pressure-driven aerosol nebulizer or an ultrasonic nebulizer, as is known to those of skill in the art. See, e.g., U.S. Pat. No. 4,501,729. Aerosols of solid particles (e.g., lyophilized, freeze dried, etc.) can likewise be produced with any solid particulate medicament aerosol generator, by techniques known in the pharmaceutical art. As another example, the pharmaceutical compositions can be formulated as an on-demand dissolvable form, which provides a lyophilized portion of the pharmaceutical composition and a dissolving solution portion of the pharmaceutical composition.

In some embodiments, the pharmaceutical composition is in the form of an aqueous suspension, which can be prepared from solutions or suspensions. With respect to solutions or suspensions, dosage forms can be comprised of micelles of lipophilic substances, liposomes (phospholipid vesicles/membranes) and/or a fatty acid (e.g., palmitic acid). In particular embodiments, the pharmaceutical composition is a solution or suspension that is capable of dissolving in the fluid secreted by mucous membranes of the epithelium of the tissue to which it is administered, applied and/or delivered, which can advantageously enhance absorption.

The pharmaceutical composition can be an aqueous solution, a nonaqueous solution or a combination of an aqueous and nonaqueous solution. Suitable aqueous solutions include, but are not limited to, aqueous gels, aqueous suspensions, aqueous microsphere suspensions, aqueous microsphere dispersions, aqueous liposomal dispersions, aqueous micelles of liposomes, aqueous microemulsions, and any combination of the foregoing, or any other aqueous solution that can dissolve in the fluid secreted by the mucosal membranes of the nasal cavity. Exemplary nonaqueous solutions include, but are not limited to, nonaqueous gels, nonaqueous suspensions, nonaqueous microsphere suspensions, nonaqueous microsphere dispersions, nonaqueous liposomal dispersions, nonaqueous emulsions, nonaqueous microemulsions, and any combination of the foregoing, or any other nonaqueous solution that can dissolve or mix in the fluid secreted by mucosal membranes.

Examples of powder formulations include, without limitation, simple powder mixtures, micronized powders, freeze dried powder, lyophilized powder, powder microspheres, coated powder microspheres, liposomal dispersions, and any combination of the foregoing. Powder microspheres can be formed from various polysaccharides and celluloses, which include without limitation starch, methylcellulose, xanthan gum, carboxymethylcellulose, hydroxypropyl cellulose, carbomer, alginate polyvinyl alcohol, acacia, chitosans, and any combination thereof.

In particular embodiments, the composition is one that is at least partially, or even substantially (e.g., at least 80%, 90%, 95% or more) soluble in the fluids that are secreted by mucosa so as to facilitate absorption. Alternatively or additionally, the composition can be formulated with a carrier and/or other substances that foster dissolution of the agent within secretions, including without limitation fatty acids (e.g., palmitic acid), gangliosides (e.g., GM-1), phospholipids (e.g., phosphatidylserine), and emulsifiers (e.g., polysorbate 80).

Those skilled in the art will appreciate that for intranasal administration or delivery, because the volume of the pharmaceutical composition administered is generally small, nasal secretions may alter the pH of the administered dose since the range of pH in the nasal cavity can be as wide as 5 to 8. Such alterations can affect the concentration of un-ionized drug available for absorption. Accordingly, in representative embodiments, the pharmaceutical composition further comprises a buffer to maintain or regulate pH in situ. Typical buffers include, but are not limited to, ascorbate, acetate, citrate, prolamine, carbonate, and phosphate buffers.

In embodiments, the pH of the pharmaceutical composition is selected so that the internal environment of the mucosal tissue after administration is on the acidic to neutral side, which (1) can provide the active compound in an un-ionized form for absorption, (2) prevents growth of pathogenic bacteria, which is more likely to occur in an alkaline environment, and (3) reduces the likelihood of irritation of the mucosa.

For liquid and powder sprays or aerosols, the pharmaceutical composition can be formulated to have any suitable and desired particle or droplet size. In illustrative embodiments, the majority and/or the mean size of the particles or droplets range from equal to or greater than about 1, 2.5, 5, 10, 15 or 20 microns and/or equal to or less than about 25, 30, 40, 45, 50, 60, 75, 100, 125, 150, 175, 200, 225, 250, 275, 300, 325, 350, 375, 400, or 425 microns (including all combinations of the foregoing). Representative examples of suitable ranges for the majority and/or mean particle or droplet size include, without limitation, from about 5 to 100 microns, from about 10 to 60 microns, from about 175 to 325 microns, and from about 220 to 300 microns which facilitate the deposition of a safe and effective amount of the active compound, for example, in the nasal cavity (e.g., in the upper third of the nasal cavity, the superior meatus, the olfactory region and/or the sinus region to target the olfactory neural pathway). In general, particles or droplets smaller than about 5 microns will be deposited in the trachea or even the lung, whereas particles or droplets that are about 50 microns or larger generally do not reach the nasal cavity and are deposited in the anterior nose.

International patent publication WO 2005/023335 (Kurve Technology, Inc.) describes particles and droplets having a diameter size suitable for the practice of representative embodiments of pharmaceutical compositions disclosed herein. In particular embodiments, the particles or droplets have a mean diameter of about 5 to 30 microns, about 10 to 20 microns, about 10 to 17 microns, about 10 to 15 microns, about 12 to 17 microns, about 10 to 15 microns or about 10 to 12 microns. The particles can “substantially” have a mean diameter or size as described herein, i.e., at least about 50%, 60%, 70%, 80%, 90% or 95 or more of the particles are of the indicated diameter or size range.

The pharmaceutical composition can be delivered as a nebulized or atomized liquid having a droplet size as described above.

According to particular embodiments of this disclosure that comprise methods of intranasal delivery, it can be desirable to prolong the residence time of the pharmaceutical composition in the nasal cavity (e.g., in the upper third of the nasal cavity, the superior meatus, the olfactory region and/or in the sinus region), for example, to enhance absorption. Thus, the pharmaceutical composition can optionally be formulated with a bioadhesive polymer, a gum (e.g., xanthan gum), chitosan (e.g., highly purified cationic polysaccharide), pectin (or any carbohydrate that thickens like a gel or emulsifies when applied to nasal mucosa), a microsphere (e.g., starch, albumin, dextran, cyclodextrin), gelatin, a liposome, carbamer, polyvinyl alcohol, alginate, acacia, chitosans and/or cellulose (e.g., methyl or propyl; hydroxyl or carboxy; carboxymethyl or hydroxylpropyl), which are agents that enhance residence time in the nasal cavity. As a further approach, increasing the viscosity of the formulation can also provide a means of prolonging contact of the agent with the nasal epithelium. The pharmaceutical composition can be formulated as a nasal emulsion, ointment or gel, which offers advantages for local application because of their viscosity.

Moist and highly vascularized membranes can facilitate rapid absorption; consequently, the pharmaceutical composition can optionally comprise a humectant, particularly in the case of a gel-based composition so as to assure adequate intranasal moisture content. Examples of suitable humectants include but are not limited to glycerin or glycerol, mineral oil, vegetable oil, membrane conditioners, soothing agents, and/or sugar alcohols (e.g., xylitol, sorbitol; and/or mannitol). The concentration of the humectant in the pharmaceutical composition will vary depending upon the agent selected and the formulation.

The pharmaceutical composition can also optionally include an absorption enhancer, such as an agent that inhibits enzyme activity, reduces mucous viscosity or elasticity, decreases mucociliary clearance effects, opens tight junctions, and/or solubilizes the active compound. Chemical enhancers are known in the art and include chelating agents (e.g., EDTA), fatty acids, bile acid salts, surfactants, and/or preservatives. Enhancers for penetration can be particularly useful when formulating compounds that exhibit poor membrane permeability, lack of lipophilicity, and/or are degraded by aminopeptidases. The concentration of the absorption enhancer in the pharmaceutical composition will vary depending upon the agent selected and the formulation.

To extend shelf life, preservatives can optionally be added to the pharmaceutical composition. Suitable preservatives include but are not limited to benzyl alcohol, parabens, thimerosal, chlorobutanol and benzalkonium chloride, and combinations of the foregoing. The concentration of the preservative will vary depending upon the preservative used, the compound being formulated, the formulation, and the like. In representative embodiments, the preservative is present in an amount of about 2% by weight or less.

The pharmaceutical compositions described herein can optionally contain an odorant, e.g., as described in EP 0 504 263 B1, to provide a sensation of odor, to aid in inhalation of the composition so as to promote delivery to the olfactory region and/or to trigger transport by the olfactory neurons.

As another option, the composition can comprise a flavoring agent, e.g., to enhance the taste and/or acceptability of the composition to the subject.

### Porous Particles for Pulmonary Administration

In some embodiments, the particles are porous, so that they have an appropriate density to avoid deposition in the back of the throat when administered via an inhaler. The combination of relatively large particle size and relatively low density avoids phagocytosis in the lungs, provides appropriately targeted delivery, avoids systemic delivery of the components, and provides a high concentration of the components in the lung.

Representative methods for preparing such particles, and for delivering such particles, are described, for example, in U.S. Pat. No. 7,384,649, entitled, “Particulate compositions for pulmonary delivery,” U.S. Pat. No. 7,182,961, entitled “Particulate compositions for pulmonary delivery,” U.S. Pat. No. 7,146,978, entitled, “Inhalation device and method,” U.S. Pat. No. 7,048,908, entitled “Particles for inhalation having sustained release properties,” U.S. Pat. No. 6,956,021, entitled “Stable spray-dried protein formulations,” U.S. Pat. No. 6,766,799, entitled “Inhalation device,” and U.S. Pat. No. 6,732,732, entitled “Inhalation device and method.”

Additional patents disclosing such particles include U.S. Pat. No. 7,279,182, entitled “Formulation for spray-drying large porous particles,” U.S. Pat. No. 7,252,840, entitled “Use of simple amino acids to form porous particles,” U.S. Pat. No. 7,032,593, entitled “Inhalation device and method,” U.S. Pat. No. 7,008,644, entitled “Method and apparatus for producing dry particles,” U.S. Pat. No. 6,848,197, entitled “Control of process humidity to produce large, porous particles,” and U.S. Pat. No. 6,749,835, entitled “Formulation for spray-drying large porous particles.”

U.S. Pat. No. 7,678,364, entitled “Particles for inhalation having sustained release properties,” discloses methods for delivering particles to the pulmonary system comprising: administering to the respiratory tract of a patient in need of treatment, prophylaxis or diagnosis a safe and effective amount of a dry powder comprising: a) a multivalent metal cation which is complexed with a therapeutic, prophylactic or diagnostic agent; b) a pharmaceutically acceptable carrier; and c) a multivalent metal cation-containing component wherein the dry powder is spray-dried and has a total amount of multivalent metal cation which is about 10% w/w or more of the total weight of the agent, a tap density of about 0.4 g/cm³ or less, a median geometric diameter of from about 5 micrometers to about 30 micrometers and an aerodynamic diameter of from about 1 to about 5 microns.

The amount of the compounds described herein, or salts thereof, present in the particles can range from about 0.1 weight % to about 95 weight %, though in some cases, can even be as high as 100%. For example, from about 1 to about 50%, such as from about 5 to about 30%. Particles in which the compound is distributed throughout a particle can be preferred.

In some embodiments, the particles include a surfactant other than the phospholipids described above. As used herein, the term “surfactant” refers to any agent which preferentially absorbs to an interface between two immiscible phases, such as the interface between water and an organic polymer solution, a water/air interface or organic solvent/air interface. Surfactants generally possess a hydrophilic moiety and a lipophilic moiety, such that, upon absorbing to particles, they tend to present moieties to the external environment that do not attract similarly-coated particles, thus reducing particle agglomeration. Surfactants may also promote absorption of a therapeutic or diagnostic agent and increase bioavailability of the agent.

Suitable surfactants which can be employed in fabricating the particles disclosed herein include but are not limited to hexadecanol; fatty alcohols such as polyethylene glycol (PEG); polyoxyethylene-9-lauryl ether; a surface active fatty acid, such as palmitic acid or oleic acid; glycocholate; surfactin; a poloxamer; a sorbitan fatty acid ester such as sorbitan trioleate (Span 85); Tween® 80 and tyloxapol.

The surfactant can be present in the particles in an amount ranging from about 0 to about 5 weight %. Preferably, it can be present in the particles in an amount ranging from about 0.1 to about 1.0 weight %.

Particles that have a tap density less than about 0.4 g/cm³, median diameters of at least about 5 μm, and an aerodynamic diameter of from about 1 μm to about 5 μm, or from about 1 μm to about 3 μm, are more capable of escaping inertial and gravitational deposition in the oropharyngeal region, and are targeted to the airways or the deep lung. The use of larger, more porous particles is advantageous since they are able to aerosolize more efficiently than smaller, denser aerosol particles such as those currently used for inhalation therapies.

### Liposomal Delivery

The compositions described herein are advantageously delivered to the lungs, so as to provide the compounds at the site of an actual or potential norovirus or coronavirus infection. This can be accomplished by pulmonary delivery via metered-dose inhalers or other pulmonary delivery devices, and also by lodging particles in the capillary beds surrounding the alveoli in the lungs.

Nanocarriers, such as liposomes, including small unilamellar vesicles, show several advantages over other conventional approaches for delivering drugs to the lungs, including prolonged drug release and cell-specific targeted drug delivery. Nano-sized drug carriers can also be advantageous for delivering poorly water soluble drugs, and certain of the compounds described herein are poorly water-soluble. Additional advantages include their ability to provide controlled release, protection from metabolism and degradation, decreased drug toxicity and targeting capabilities.

The liposomes (preferably unilamellar vesicles) have a size less than 200 nm as measured by dynamic light scattering, and preferably characterized by being comprised of chemically pure synthetic phospholipids, most preferably having aliphatic side chains of a length of at least 16 carbons, and containing one or more of the compounds described herein, or a pharmaceutically acceptable salt thereof, sufficient to preferentially deliver (i.e., target) a quantity of the compounds thereof to the capillary beds surrounding the alveoli. Vesicle diameter can be measured, for example, by dynamic light scattering using a helium-neon 100 mW NEC gas laser and a Malvern K7027 correlator, ideally with at least two or three measurements made for each for each size determination.

The expression “chemically pure phospholipids” is meant to define phospholipids which are essentially free of deleterious detergent moieties and impurities which cause aggregation of small unilamellar vesicles (SUVs) formed therefrom, and which are more than 97% pure. Preferably, the liposomes have a diameter predominantly of from about 50 to about 160 nm, are essentially neutral in charge, and incorporate phospholipids having a side chain length of from 16 to 18 carbon atoms. More preferably, the liposomes are prepared from distearoyl phosphatidylcholine (DSPC) and include cholesterol (most preferably in an amount of from 10 to 50% of total lipid) as a vesicle stabilizer.

It can also be advantageous that the liposomes have a melting point above body temperature (i.e., above 37° C.). For this reason, it can be advantageous to use pure phospholipids, preferably ones that are saturated, and have a carbon chain length of at least 16 carbons, preferably between 16 and 18 carbons. Distearoylphosphatidyl choline (DSPC) is a preferred phospholipid.

Cholesterol helps to stabilize the liposomes, and is preferably added in a sufficient amount to provide liposome stability. Most preferably, the liposomes further comprise a pegylated phospholipid, such as DSPEPEG. The method involves introducing into a patient's bloodstream an amount of liposomes, of a size of less than 200 nm (preferably unilamellar vesicles) and preferably characterized by being comprised of chemically pure synthetic phospholipids, most preferably having aliphatic side chains of a length of at least 16 carbons, and containing the compounds described herein, or a pharmaceutically acceptable salt or prodrug thereof, sufficient to preferentially deliver (i.e., target) a quantity of the compounds to the capillary beds in the lungs, surrounding the alveoli.

The compounds described herein can be combined with other anti-norovirus or anti-coronavirus agents. Such additional agents can also be present in the liposomes, can be present in different liposomes, or can be co-administered via a different route.

The liposomes include one or more of the compounds described herein, or a pharmaceutically acceptable salt thereof, and can optionally include other anti-norovirus or anti-coronavirus agents. The liposomes can be prepared by dissolving the phospholipid and cholesterol in an appropriate organic solvent, such as chloroform, and evaporating the solvent to form a lipid film. If an ionophore is employed to load the compounds described herein into the liposomes, the ionophore may be added to the lipid solution before evaporation. The dried lipid film is then rehydrated in an appropriate aqueous phase, such as phosphate-buffered saline or other physiologically appropriate solution. Water-soluble drugs or therapeutic agents may be contained in the hydrating solution, although if remote loading is desired a loading agent such as a chelating agent described above may be added to the hydrating solution to be encapsulated within the inner aqueous space of the liposome.

Upon the addition of the hydrating solution, liposomes of varying size spontaneously form and encapsulate a portion of the aqueous phase. Thereafter, the liposomes and suspending aqueous solution are subjected to a shear force such as extrusion, sonication, or processing through a homogenizer according to the method described in U.S. Pat. No. 4,753,788; to produce vesicles within the specified size.

The liposomes can then be processed to remove undesirable compounds from the suspending solution, for example, un-encapsulated drug, which may be accomplished through processes such as gel chromatography or ultrafiltration.

The use of liposomes in dry powder aerosols for targeted lung delivery is described, for example, in Willis et al., Lung, June 2012, 190(3):251-262. One advantage is that the phospholipids used to prepare the liposomes are similar to endogenous lung surfactant.

### Routes of Administration and Dosages

The compounds and pharmaceutically acceptable compositions described above can be administered to humans and other animals orally, rectally, parenterally, intracisternally, intravaginally, intraperitoneally, topically (as by powders, ointments, or drops), bucally, as an oral or nasal spray, to the pulmonary system, such as by using an inhaler, such as a metered dose inhaler (MDI), or the like, depending on the severity of the infection being treated. In some embodiments, the compound or composition disclosed herein is administered orally, via inhalation, or intravenously.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are specifically suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound described herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions described herein may be orally administered in any orally acceptable dosage form including, but not limited to, capsules, tablets, aqueous suspensions or solutions. In the case of tablets for oral use, carriers commonly used include, but are not limited to, lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions described herein may be administered in the form of suppositories for rectal administration. These can be prepared by mixing the agent with a suitable non-irritating excipient which is solid at room temperature but liquid at rectal temperature and therefore will melt in the rectum to release the drug. Such materials include, but are not limited to, cocoa butter, beeswax and polyethylene glycols.

The pharmaceutical compositions described herein may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topical application also includes the use of transdermal patches.

For topical applications, the pharmaceutical compositions may be formulated in a suitable ointment containing the active component suspended or dissolved in one or more carriers. Carriers for topical administration of the compounds of this disclosure include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

For ophthalmic use, the pharmaceutical compositions may be formulated as micronized suspensions in isotonic, pH adjusted sterile saline, or, specifically, as solutions in isotonic, pH adjusted sterile saline, either with or without a preservative such as benzalkonium chloride. Alternatively, for ophthalmic uses, the pharmaceutical compositions may be formulated in an ointment such as petrolatum.

The pharmaceutical compositions may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The compounds for use in the methods of the disclosure can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

### III. Methods of Treatment

Provided herein are uses of a compound described herein as a therapeutic agent. The compounds described herein or pharmaceutically acceptable salts thereof can be used to reduce viral titer in a biological sample (e.g. an infected cell culture) or in humans (e.g. lung viral titer in a patient). The compounds described herein or pharmaceutically acceptable salts thereof can be used in methods of treating viral infections. Non-limiting examples of viral infections which can be treated with the compounds described herein or their pharmaceutically acceptable salts include coronavirus infections, calicivirus infections, and picornavirus infections.

Non-limiting examples of calicivirus infections include norovirus mediated conditions and norovirus infection. The terms “norovirus mediated condition”, “norovirus infection”, and “norovirus”, as used herein, are used interchangeably to mean the disease caused by an infection with a norovirus.

Noroviruses are infectious viruses that cause gastroenteritis in mammals. Noroviruses are RNA viruses of the family Caliciviridae, which comprises seven genogroups: GI, GII, GIII, GIV, GV, GVI, and GVII. Genogroup II, the most prevalent human genogroup, presently contains 19 genotypes. Genogroups 1, 11 and IV infect humans, whereas genogroup Ill infects bovine species, and genogroup V has recently been isolated in mice. The two groups most associated with gastroenteritis in humans are genogroup I (GI), which includes Norwalk virus, Desert Shield virus and Southampton virus; and genogroup II (GII), which includes Bristol virus, Lordsdale virus, Toronto virus, Mexico virus, Hawaii virus and Snow Mountain virus.

In some embodiments, the compounds used herein are for treatment of noroviruses which are associated with gastroenteritis. In some embodiments, noroviruses are associated with Norwalk virus. In some embodiments, noroviruses are associated with HuNV GGII.4.

In some embodiments, the compounds disclosed herein can be used in the treatment of norovirus, wherein the compound binds to free virus, or inhibits a norovirus protease. In some cases, the compound can target both (free virus and protease).

In humans, common symptoms of norovirus are nausea, vomiting, watery diarrhea, abdominal pain, and in some cases, loss of taste. Norovirus can establish a long term infection in people who are immunocompromised. In severe cases, persistent infections can lead to norovirus-associated enteropathy, intestinal villous atrophy, and malabsorption. Norovirus-associated gastroenteritis is also called “winter vomiting bug”.

A person usually develops symptoms of gastroenteritis 12 to 48 hours after being exposed to norovirus. General lethargy, weakness, muscle aches, headaches, and low-grade fevers may occur.

The terms “coronavirus mediated condition” and “coronavirus infection” as used herein, are used interchangeably to mean the disease caused by an infection with a coronavirus. Non-limiting examples of coronaviruses include severe acute respiratory syndrome-related coronavirus (SARS), Middle East respiratory syndrome-related coronavirus (MERS), and SARS-CoV-2 virus (also known as 2019-nCoV, or Wuhan coronavirus). Non-limiting examples of coronavirus mediated conditions or coronavirus infections include SARS, MERS, and COVID-19.

Coronaviruses are a family of viruses that cause diseases in mammals and birds. Coronaviruses are in the subfamily Orthocoronavirinae in the family Coronaviridae, in the order Nidovirales. There are four main genera of coronaviruses, known as alpha, beta, gamma, and delta. Coronaviruses that affect humans include Human coronavirus 229E (HCoV-229E), Human coronavirus OC43 (HCoV-OC43), Severe acute respiratory syndrome-related coronavirus (SARS-CoV), Human coronavirus NL63 (HCoV-NL63, New Haven coronavirus), Human coronavirus HKU1, Middle East respiratory syndrome-related coronavirus (MERS-CoV, previously known as novel coronavirus 2012 and HCoV-EMC), and SARS-CoV-2 (also known as 2019-nCoV and Wuhan coronavirus).

In humans, coronaviruses cause respiratory infections, including the common cold, which are typically mild, though rarer forms such as SARS, MERS and SARS-CoV-2 (the cause of the 2019-20 COVID-19 outbreak) can be lethal. Symptoms vary in other species: in chickens, they cause an upper respiratory disease, while in cows and pigs coronaviruses cause diarrhea. There are no vaccines or antiviral drugs to prevent or treat human coronavirus infections. The coronaviruses HCoV-229E, -NL63, -OC43, and -HKU1 continually circulate in the human population and cause respiratory infections in adults and children worldwide

In some embodiments, the compounds used herein are for treatment of alphacoronaviruses or betacoronaviruses. In some cases, the compounds used herein are for treatment of alphacoronaviruses. Non-limiting examples of alphacoronaviruses include HCoV-229E and HCoV-NL63. In some embodiments, the compounds used herein are for treatment of betacoronaviruses. Non-limiting examples of betacoronaviruses are HCoV-HKU1, HCoV-OC43, Middle East respiratory syndrome coronavirus (MERS-CoV), the severe acute respiratory syndrome coronavirus (SARS-CoV), and SARS-CoV-2. In some embodiments, the compounds used herein are for treatment of coronaviruses which are associated with SARS, MERS, and COVID-19. In some embodiments, coronaviruses are associated with SARS. In some embodiments, coronaviruses are associated with MERS. In some embodiments, coronaviruses are associated with COVID-19.

In some embodiments, the compounds disclosed herein can be used in the treatment of coronavirus, wherein the compound binds to free virus, or inhibits a coronavirus protease. In some cases, the compound can target both (free virus and protease).

In humans, common symptoms of coronavirus are fever, cough, shortness of breath, and myalgia.

Non-limiting examples of picornavirus infections include rhinovirus mediated conditions and rhinovirus infections. The terms “rhinovirus mediated condition” and “rhinovirus infection” as used herein, are used interchangeably to mean the disease caused by an infection with a rhinovirus.

Picornaviruses infect both humans and animals, can cause severe paralysis (paralytic poliomyelitis), aseptic meningitis, hepatitis, pleurodynia, myocarditis, skin rashes, and colds; although asymptomatic infection is common. Several medically important genera are members of this family, such as enterovirus (including poliovirus (PV), rhinoviruses, and human enteroviruses (e.g. coxsackie viruses)); hepatovirus which includes hepatitis A virus (HAV); and aphthoviruses which include the foot- and mouth disease virus (FMDV). Rhinoviruses are recognized as the principle cause of the common cold in humans, and comprise three different species: A, B, and C. Transmission is primarily by the aerosol route and the virus replicates in the nose.

In some embodiments, the compounds disclosed herein can be used in the treatment of picornavirus infection. In some embodiments, the compounds disclosed herein can be used in the treatment of rhinovirus infection. In some embodiments, the compounds disclosed herein can be used in the treatment of rhinovirus infection wherein the compound binds to free virus, or inhibits a rhinovirus protease. In some cases, the compound can target both (free virus and protease).

The terms, “disease”, “disorder”, and “condition” may be used interchangeably here to refer to norovirus or coronavirus virus mediated medical or pathological condition.

As used herein, the terms “subject” and “patient” are used interchangeably. The terms “subject” and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In one embodiment, the subject is a non-human animal such as a farm animal (e.g., a horse, cow, pig or sheep), or a pet (e.g., a dog, cat, guinea pig or rabbit). In a preferred embodiment, the subject is a “human”.

The term “biological sample”, as used herein, includes, without limitation, cell cultures or extracts thereof; biopsied material obtained from a mammal or extracts thereof; blood, saliva, urine, feces, semen, tears, or other body fluids or extracts thereof.

As used herein, “multiplicity of infection” or “MOI” is the ratio of infectious agents (e.g. phage or virus) to infection targets (e.g. cell). For example, when referring to a group of cells inoculated with infectious virus particles, the multiplicity of infection or MOI is the ratio defined by the number of infectious virus particles deposited in a well divided by the number of target cells present in that well.

As used herein the terms “inhibition of the replication of noroviruses” and “inhibition of the replication of coronaviruses” includes both the reduction in the amount of virus replication (e.g. the reduction by at least 10%) and the complete arrest of virus replication (i.e., 100% reduction in the amount of virus replication). In some embodiments, the replication of norovirus or coronavirus viruses are inhibited by at least 50%, at least 65%, at least 75%, at least 85%, at least 90%, or at least 95%.

Norovirus or coronavirus virus replication can be measured by any suitable method known in the art. For example, norovirus or coronavirus viral titer in a biological sample (e.g. an infected cell culture) or in humans (e.g. lung viral titer in a patient) can be measured. More specifically, for cell based assays, in each case cells are cultured in vitro, virus is added to the culture in the presence or absence of a test agent, and after a suitable length of time a virus-dependent endpoint is evaluated. Such assays are known in the art. A first type of cell assay that can be used in the disclosure depends on death of the infected target cells, a process called cytopathic effect (CPE), where virus infection causes exhaustion of the cell resources and eventual lysis of the cell. In the first type of cell assay, a low fraction of cells in the wells of a microtiter plate are infected (typically 1/10 to 1/1000), the virus is allowed to go through several rounds of replication over 48-72 hours, then the amount of cell death is measured using a decrease in cellular ATP content compared to uninfected controls. A second type of cell assay that can be employed in the disclosure depends on the multiplication of virus-specific RNA molecules in the infected cells, with RNA levels being directly measured using the branched-chain DNA hybridization method (bDNA). In the second type of cell assay, a low number of cells are initially infected in wells of a microtiter plate, the virus is allowed to replicate in the infected cells and spread to additional rounds of cells, then the cells are lysed and viral RNA content is measured. This assay is stopped early, usually after 18-36 hours, while all the target cells are still viable. Viral RNA is quantitated by hybridization to specific oligonucleotide probes fixed to wells of an assay plate, then amplification of the signal by hybridization with additional probes linked to a reporter enzyme.

As used herein a “viral titer (or titer)” is a measure of virus concentration. Titer testing can employ serial dilution to obtain approximate quantitative information from an analytical procedure that inherently only evaluates as positive or negative. The titer corresponds to the highest dilution factor that still yields a positive reading; for example, positive readings in the first 8 serial twofold dilutions translate into a titer of 1:256. To determine the titer, several dilutions will be prepared, such as 10⁻¹, 10⁻², 10⁻³, 10⁻⁸.

As used herein, the terms “treat”, “treatment” and “treating” refer to both therapeutic and prophylactic treatments. For example, therapeutic treatments includes the reduction or mitigation of the progression, severity and/or duration of norovirus or coronavirus mediated conditions, or the amelioration of one or more symptoms (specifically, one or more discernible symptoms) of norovirus or coronavirus mediated conditions, resulting from the administration of one or more therapies (e.g., one or more therapeutic agents such as a compound or composition of the disclosure). In specific embodiments, the therapeutic treatment includes the amelioration of at least one measurable physical parameter of a norovirus or coronavirus mediated condition. In other embodiments the therapeutic treatment includes the inhibition of the progression of a norovirus or coronavirus mediated condition, either physically by, e.g., stabilization of a discernible symptom, physiologically by, e.g., stabilization of a physical parameter, or both. In other embodiments the therapeutic treatment includes the reduction or stabilization of norovirus or coronavirus mediated infections. Antiviral drugs can be used in the community setting to treat people who already have norovirus or coronavirus to reduce the severity of symptoms and reduce the number of days that they are sick.

The term “chemotherapy” refers to the use of medications, e.g. small molecule drugs (rather than “vaccines”) for treating a disorder or disease.

The terms “prophylaxis” or “prophylactic use” and “prophylactic treatment” as used herein, refer to any medical or public health procedure whose purpose is to prevent, rather than treat or cure a disease. As used herein, the terms “prevent”, “prevention” and “preventing” refer to the reduction in the risk of acquiring or developing a given condition, or the reduction or inhibition of the recurrence or said condition in a subject who is not ill, but who has been or may be near a person with the disease. The term “chemoprophylaxis” refers to the use of medications, e.g. small molecule drugs (rather than “vaccines”) for the prevention of a disorder or disease.

As used herein, prophylactic use includes the use in situations in which an outbreak has been detected, to prevent contagion or spread of the infection in places where a lot of people that are at high risk of serious norovirus or coronavirus complications live in close contact with each other (e.g. in a hospital ward, daycare center, prison, nursing home, etc.). It also includes the use among populations who require protection from the norovirus or coronavirus but who either do not get protection after vaccination (e.g. due to weak immune system), or when the vaccine is unavailable to them, or when they cannot get the vaccine because of side effects. It also includes use during the two weeks following vaccination, since during that time the vaccine is still ineffective. Prophylactic use may also include treating a person who is not ill with the norovirus or coronavirus or not considered at high risk for complications, in order to reduce the chances of getting infected with norovirus or coronavirus and passing it on to a high-risk person in close contact with him (for instance, healthcare workers, nursing home workers, etc.).

In some embodiments, the methods of the disclosure are a preventative or “prophylactic” measure to a patient, specifically a human, having a predisposition to complications resulting from infection by a norovirus or coronavirus virus. Prophylactic use includes use in situations in which an “index case” or an “outbreak” has been confirmed, in order to prevent the spread of infection in the rest of the community or population group.

In embodiments, the methods of the disclosure are applied as a “prophylactic” measure to members of a community or population group, specifically humans, in order to prevent the spread of infection.

As used herein, an “effective amount” refers to an amount sufficient to elicit the desired biological response. In the present disclosure the desired biological response is to inhibit the replication of norovirus or coronavirus, to reduce the amount of norovirus or coronavirus or to reduce or ameliorate the severity, duration, progression, or onset of a norovirus or coronavirus virus infection, prevent the advancement of a norovirus or coronavirus infection, prevent the recurrence, development, onset or progression of a symptom associated with a norovirus or coronavirus infection, or enhance or improve the prophylactic or therapeutic effect(s) of another therapy used against norovirus or coronavirus infections. The precise amount of compound administered to a subject will depend on the mode of administration, the type and severity of the infection and on the characteristics of the subject, such as general health, age, sex, body weight and tolerance to drugs. The skilled artisan will be able to determine appropriate dosages depending on these and other factors. When co-administered with other anti-viral agents, e.g., when co-administered with an anti-norovirus or coronavirus medication, an “effective amount” of the second agent will depend on the type of drug used. Suitable dosages are known for approved agents and can be adjusted by the skilled artisan according to the condition of the subject, the type of condition(s) being treated and the amount of a compound described herein being used. In cases where no amount is expressly noted, a safe and effective amount should be assumed. For example, compounds described herein can be administered to a subject in a dosage range from between approximately 0.01 to 100 mg/kg body weight/day for therapeutic or prophylactic treatment.

Generally, dosage regimens can be selected in accordance with a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the renal and hepatic function of the subject; and the particular compound or salt thereof employed, the duration of the treatment; drugs used in combination or coincidental with the specific compound employed, and like factors well known in the medical arts. The skilled artisan can readily determine and prescribe the effective amount of the compounds described herein required to treat, to prevent, inhibit (fully or partially) or arrest the progress of the disease.

Dosages of the compounds for uses described herein can range from between about 0.01 to about 100 mg/kg body weight/day, about 0.01 to about 50 mg/kg body weight/day, about 0.1 to about 50 mg/kg body weight/day, or about 1 to about 25 mg/kg body weight/day. It is understood that the total amount per day can be administered in a single dose or can be administered in multiple dosing, such as twice a day (e.g., every 12 hours), three times a day (e.g., every 8 hours), or four times a day (e.g., every 6 hours).

For therapeutic treatment, the compounds described herein can be administered to a patient within, for example, 48 hours (or within 40 hours, or less than 2 days, or less than 1.5 days, or within 24 hours) of onset of symptoms (e.g., nasal congestion, sore throat, cough, aches, fatigue, headaches, and chills/sweats). The therapeutic treatment can last for any suitable duration, for example, for 5 days, 7 days, 10 days, 14 days, etc. For prophylactic treatment during a community outbreak, the compounds described herein can be administered to a patient within, for example, 2 days of onset of symptoms in the index case, and can be continued for any suitable duration, for example, for 7 days, 10 days, 14 days, 20 days, 28 days, 35 days, 42 days, etc.

### Combination Therapy

The compounds described herein can be used in combination therapy, i.e., in conjunction with other anti-norovirus or anti-coronavirus compounds, or in conjunction with a vaccine. Combination therapy can be particularly advantageous where a patient might be exposed to more than one form of the norovirus or coronavirus virus.

A safe and effective amount can be achieved in the method or pharmaceutical composition of the disclosure employing a compound of Formulas I, IA, II, IIA, IIB, IIC, or IID, or a pharmaceutically acceptable salt thereof alone or in combination with an additional suitable therapeutic agent, for example, an antiviral agent or a vaccine. When “combination therapy” is employed, a safe and effective amount can be achieved using a first amount of a compound of Formulas I, IA, II, IIA, IIB, IIC, or IID, or a pharmaceutically acceptable salt thereof, and a second amount of an additional suitable therapeutic agent (e.g. an antiviral agent or vaccine).

In embodiments, the compound of Formulas I, IA, II, IIA, IIB, IIC, or IID, or a pharmaceutically acceptable salt, and the additional therapeutic agent, are each administered in a safe and effective amount (i.e., each in an amount which would be therapeutically effective if administered alone). In other embodiments, the compound of Formulas I, IA, II, IIA, IIB, IIC, or IID, or a pharmaceutically acceptable salt thereof, and the additional therapeutic agent, are each administered in an amount which alone does not provide a therapeutic effect (a sub-therapeutic dose). In yet other embodiments, the compound of Formulas I, IA, II, IIA, IIB, IIC, or IID, or a pharmaceutically acceptable salt thereof can be administered in a safe and effective amount, while the additional therapeutic agent is administered in a sub-therapeutic dose. In still other embodiments, the compound of Formulas I, IA, II, IIA, IIB, IIC, or IID, a pharmaceutically acceptable salt thereof can be administered in a sub-therapeutic dose, while the additional therapeutic agent, for example, a suitable antiviral therapeutic agent is administered in a safe and effective amount.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a subject.

Coadministration encompasses administration of the first and second amounts of the compounds of the coadministration in an essentially simultaneous manner, such as in a single pharmaceutical composition, for example, capsule or tablet having a fixed ratio of first and second amounts, or in multiple, separate capsules or tablets for each. In addition, such coadministration also encompasses use of each compound in a sequential manner in either order.

In embodiments, the present disclosure is directed to methods of combination therapy for inhibiting the virus's replication in biological samples or patients, or for treating or preventing norovirus or coronavirus infections in patients using the compounds or pharmaceutical compositions described herein, e.g., a compound of Formulas I, IA, II, IIA, IIB, IIC, or IID, or a pharmaceutically acceptable salt thereof. Accordingly, pharmaceutical compositions also include those comprising a compound as disclosed herein (e.g., an inhibitor of virus replication) in combination with an anti-viral compound exhibiting anti-Norovirus or coronavirus virus activity.

Methods of use of the compounds and compositions disclosed herein also include combination of chemotherapy with a compound or composition of Formulas I, IA, II, IIA, IIB, IIC, or IID, or a pharmaceutically acceptable salt thereof or with a combination of a compound or composition of this disclosure with another anti-viral agent.

When co-administration involves the separate administration of the first amount of a compound of Formulas I, IA, II, IIA, IIB, IIC, or IID, or a pharmaceutically acceptable salt thereof and a second amount of an additional therapeutic agent, the compounds are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life and kinetic profile. For example, a compound of Formulas I, IA, II, IIA, IIB, IIC, or IID, or a pharmaceutically acceptable salt thereof and the second therapeutic agent can be administered in any order within about 24 hours of each other, within about 16 hours of each other, within about 8 hours of each other, within about 4 hours of each other, within about 1 hour of each other or within about 30 minutes of each other.

More, specifically, a first therapy (e.g., a prophylactic or therapeutic agent such as a compound of the disclosure) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent such as an anti-viral agent) to a subject.

It is understood that the method of co-administration of a first amount of a compound of Formulas I, IA, II, IIA, IIB, IIC, or IID, or a pharmaceutically acceptable salt thereof and a second amount of an additional therapeutic agent can result in an enhanced or synergistic therapeutic effect, wherein the combined effect is greater than the additive effect that would result from separate administration of the first amount of the compound of Formulas I, IA, II, IIA, IIB, IIC, or IID, or a pharmaceutically acceptable salt thereof and the second amount of the additional therapeutic agent.

As used herein, the term “synergistic” refers to a combination of a compound disclosed herein and another therapy (e.g., a prophylactic or therapeutic agent), which is more effective than presumed additive effects of the therapies. A synergistic effect of a combination of therapies (e.g., a combination of prophylactic or therapeutic agents) can permit the use of lower dosages of one or more of the therapies and/or less frequent administration of said therapies to a subject. The ability to utilize lower dosages of a therapy (e.g., a prophylactic or therapeutic agent) and/or to administer said therapy less frequently can reduce the toxicity associated with the administration of said therapy to a subject without reducing the efficacy of said therapy in the prevention, management or treatment of a disorder. In addition, a synergistic effect can result in improved efficacy of agents in the prevention, management or treatment of a disorder. Finally, a synergistic effect of a combination of therapies (e.g., a combination of prophylactic or therapeutic agents) may avoid or reduce adverse or unwanted side effects associated with the use of either therapy alone.

When the combination therapy using compounds as disclosed herein is in combination with a virus vaccine, both therapeutic agents can be administered so that the period of time between each administration can be longer (e.g. days, weeks or months).

The presence of a synergistic effect can be determined using suitable methods for assessing drug interaction. Suitable methods include, for example, the Sigmoid-Emax equation (Holford, N. H. G. and Scheiner, L. B., Clin. Pharmacokinet. 6: 429-453 (1981)), the equation of Loewe additivity (Loewe, S, and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied with experimental data to generate a corresponding graph to aid in assessing the effects of the drug combination. The corresponding graphs associated with the equations referred to above are the concentration-effect curve, isobologram curve and combination index curve, respectively.

### IV. Compound Preparation

Also provided herein are methods of preparing a compound as disclosed herein. In embodiments, the methods are directed to the synthesis of compounds represented by Formulas I, IA, II, IIA, IIB, IIC, or IID, or pharmaceutically acceptable salts thereof.

In general, compounds of Formula I or IA can be synthesized according to Scheme 1.

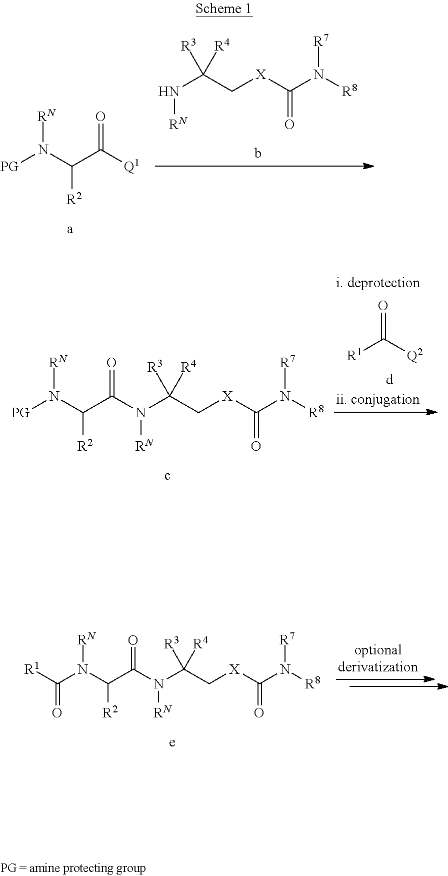

Coupling of a protected amino acid derivative a with an w-amino compound b produces amide compounds having structure c. It is understood that the moiety represented by Q¹ is reactive to the amine moiety of b such that Q¹ of a and —NHR^{N} of b form an amide bond when the coupling reaction between a and b is carried out. Subsequent deprotection of amide compound c followed by coupling with carbonyl compound d gives a polyamide compound of Formula I having a structure e. Similar to the coupling of a and b, the moiety represented by Q² in d is reactive to the amine moiety revealed by the deprotection of c such that Q² of d and —NHR^{N} of deprotected c form an amide bond when the coupling reaction between c and d is carried out. Optional subsequent derivatization gives specific compounds as described herein, e.g., compounds of Formula I or IA. Appropriate derivatization reactions can be selected based on the nature of substituents, as will be evident to a skilled chemist.

The coupling of compounds a and b and deprotected c and d can be catalyzed by appropriate reagents selected based on the precise nature of compounds a, b, c, and d. For example, when moiety Q¹ of a and/or Q² of d are hydroxyl groups (i.e., —OH), the coupling of compounds a and b (and deprotected c and d) can be catalyzed by a carbodiimide reagent e.g., 1-ethyl-3-(3-dimethylaminopropyl)carbodiimide (EDC). In embodiments, the coupling reaction may not require a catalyst, e.g., when compound a is an acyl chloride (i.e., when Q¹ is Cl).

Compounds a, b, and d can be purchased commercially or prepared by a variety of methods from commercially-available starting materials. For example, amides such as compounds having structure b can be prepared by the nucleophilic ring-opening addition of an amine (e.g., an organic primary or secondary amine such as methylamine or 1-(2,4-dimethoxyphenyl)-N-methylmethanamine) to a substituted proline derivative. Substitutions on the proline ring can be chosen to facilitate downstream derivatization of the molecule. An example of a possible synthesis of a compound b is shown in Scheme 2, below.

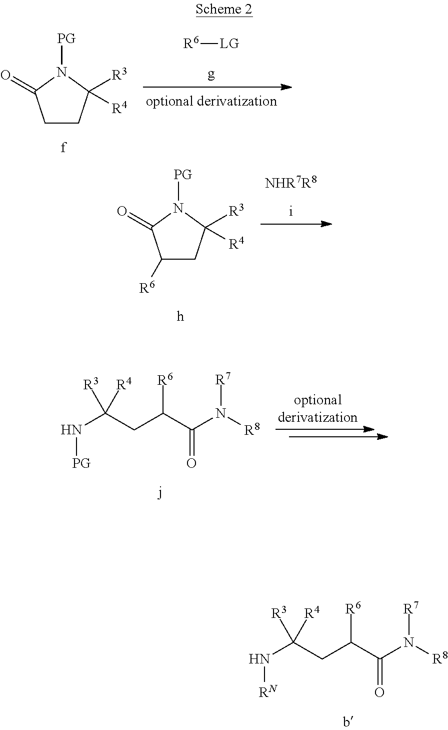

A protected proline derivative f is first optionally derivatized with a compound g having a leaving group LG (e.g., a halide or pseudohalide), e.g., through a Frater-Seebach alkylation, to form a derivative h. The proline derivative h (or f if the optional derivatization is not undertaken) is reacted with a substituted amine compound i, in the process opening the proline ring to form a compound of structure j. For example, i can be treated with a dialkylamine (e.g., methylamine) under appropriate conditions (e.g., heating in toluene) to yield a linear compound j. This compound g can be further derivatized to form a specific compound b′ analogous to compound b shown in Scheme 1. Further derivatization reactions can be chosen depending on the desired functionality, e.g., deprotection and treatment with an appropriate reagent to introduce an R^{N} moiety. The necessity of derivatizing a compound f to produce a compound of structure h can be determined by considering the nature of the compound b which will be reacted with the compound a, and the desired structure of the ultimate compound of Formula I.

The compounds of Schemes 1 and 2 above are also useful for preparing compounds of Formula II as described herein. For example, a compound of formula b′ having R⁶=alkenyl can be prepared as shown in Scheme 2, such that b′ can be reacted with a compound a′ and cyclized to form a compound of Formula II, e.g., compound k, as shown in Scheme 3.

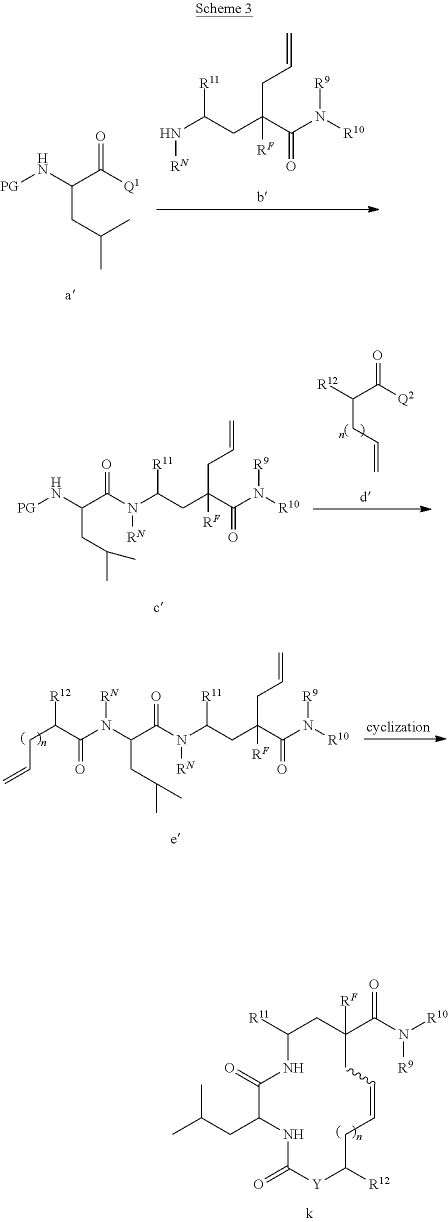

Compounds of structure k can be synthesized as shown in Scheme 3. A specific compound e′ having two alkene moieties can be prepared as shown in Scheme 1, using the ring-opening synthesis of compound b′ shown in Scheme 2. This compound e′ can undergo an intramolecular cyclization reaction, e.g., through treatment of e′ with a second-generation Grubb's catalyst in a solvent, e.g., dichloromethane, to give a cyclic compound of Formula (II) having a formula k.

The variables in these formulae are either the same as the definitions provided in the section defining the compounds described herein, or, where the functional groups defined by the variables would be labile under the reaction conditions described herein, can be either protected forms of the functional groups, or synthons for such groups. Examples of protecting groups are detailed in Greene, T. W., Wuts, P. G in “Protective Groups in Organic Synthesis”, Third Edition, John Wiley & Sons, New York: 1999 (and other editions of the book), the entire contents of which are hereby incorporated by reference.

Any suitable reaction condition known in the art, for example, in PCT WO 2005/095400 and PCT WO 2007/084557 for the coupling of a dioxaboraolan with a chloro-diazaindole can be employed. For example, the reaction between these precursors can be performed in the presence of Pd(PPh₃)₄. Specific exemplary conditions are described in the working examples in the Examples section below.

### Chiral Separations

The compounds described herein can have asymmetric centers and occur as racemates, racemic mixtures, individual diastereomers or enantiomers, with all isomeric forms being included in the present disclosure. Compounds of the present disclosure having a chiral center can exist in and be isolated in optically active and racemic forms. Some compounds can exhibit polymorphism. The present disclosure encompasses racemic, optically-active, polymorphic, or stereoisomeric forms, or mixtures thereof, of a compound of the disclosure, which possess the useful properties described herein. The optically active forms can be prepared by, for example, resolution of the racemic form by recrystallization techniques, by synthesis from optically-active starting materials, by chiral synthesis, or by chromatographic separation using a chiral stationary phase or by enzymatic resolution. One can either purify the respective compound, then derivatize the compound to form the compounds described herein, or purify the compound themselves.

Optically active forms of the compounds can be prepared using any method known in the art, including but not limited to by resolution of the racemic form by recrystallization techniques, by synthesis from optically-active starting materials, by chiral synthesis, or by chromatographic separation using a chiral stationary phase.

Examples of methods to obtain optically active materials include at least the following.

i) Physical separation of crystals: a technique whereby macroscopic crystals of the individual enantiomers are manually separated. This technique can be used if crystals of the separate enantiomers exist, i.e., the material is a conglomerate, and the crystals are visually distinct;

ii) simultaneous crystallization: a technique whereby the individual enantiomers are separately crystallized from a solution of the racemate, possible only if the latter is a conglomerate in the solid state;

iii) enzymatic resolutions: a technique whereby partial or complete separation of a racemate by virtue of differing rates of reaction for the enantiomers with an enzyme;

iv) enzymatic asymmetric synthesis: a synthetic technique whereby at least one step of the synthesis uses an enzymatic reaction to obtain an enantiomerically pure or enriched synthetic precursor of the desired enantiomer;

v) chemical asymmetric synthesis: a synthetic technique whereby the desired enantiomer is synthesized from an achiral precursor under conditions that produce asymmetry (i.e., chirality) in the product, which can be achieved using chiral catalysts or chiral auxiliaries;

vi) diastereomer separations: a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) first- and second-order asymmetric transformations: a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;

viii) kinetic resolutions: this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;

ix) enantiospecific synthesis from non-racemic precursors: a synthetic technique whereby the desired enantiomer is obtained from non-chiral starting materials and where the stereochemical integrity is not or is only minimally compromised over the course of the synthesis;

x) chiral liquid chromatography: a technique whereby the enantiomers of a racemate are separated in a liquid mobile phase by virtue of their differing interactions with a stationary phase (including but not limited to via chiral HPLC). The stationary phase can be made of chiral material or the mobile phase can contain an additional chiral material to provoke the differing interactions;

xi) chiral gas chromatography: a technique whereby the racemate is volatilized and enantiomers are separated by virtue of their differing interactions in the gaseous mobile phase with a column containing a fixed non-racemic chiral adsorbent phase;

xii) extraction with chiral solvents: a technique whereby the enantiomers are separated by virtue of preferential dissolution of one enantiomer into a particular chiral solvent;

xiii) transport across chiral membranes: a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through.

Chiral chromatography, including but not limited to simulated moving bed chromatography, is used in one embodiment. A wide variety of chiral stationary phases are commercially available.

The present disclosure will be better understood with reference to the following non-limiting examples.

### EXAMPLES

### Example 1—Synthesis of Compounds of Formula (I)

### Synthesis of Compounds A109 and A110

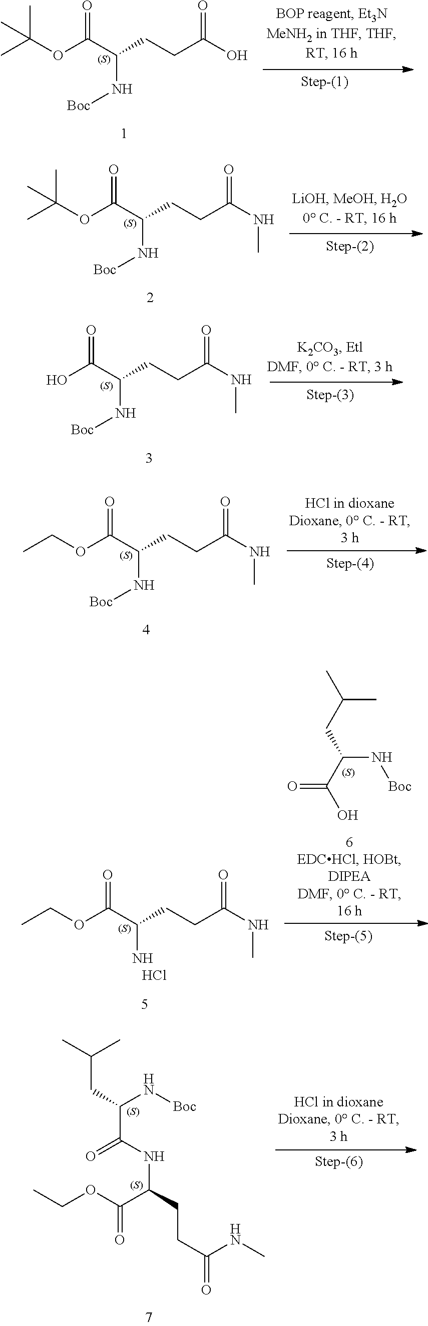

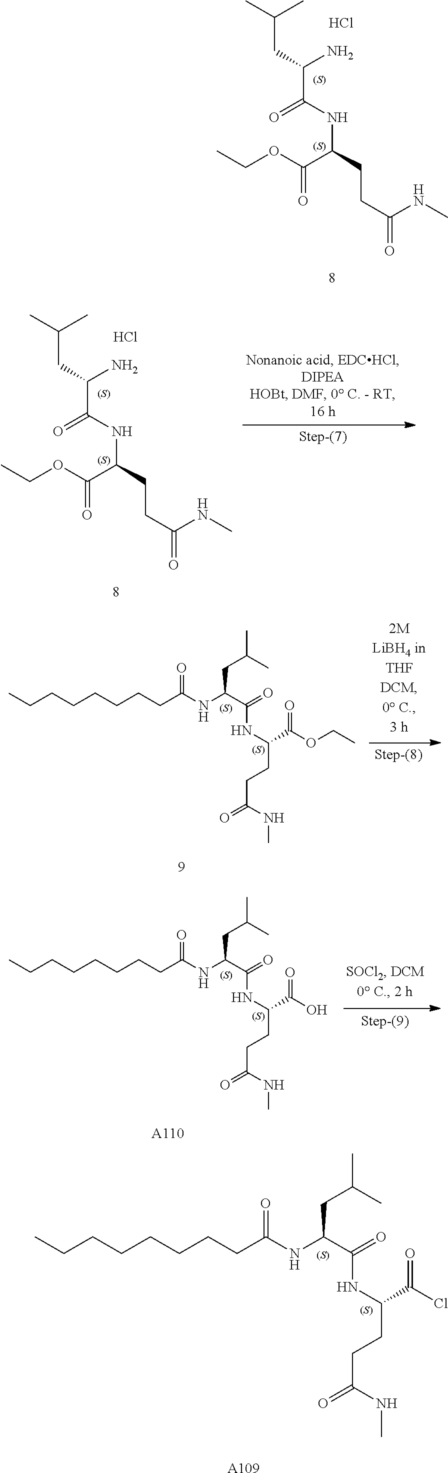

### tert-butyl N²-(tert-butoxycarbonyl)-N⁵-methyl-L-glutaminate (2)

To a stirred solution of (S)-5-(tert-butoxy)-4-((tert-butoxycarbonyl)amino)-5-oxopentanoic acid (1) (10 g, 32.96 mmol) in THE (100 mL) at 0° C. was added BOP reagent (17.49 g, 39.55 mmol), Et₃N (9.5 mL, 65.93 mmol) and stirred for 10 min, then added 2M methylamine in THE (32.96 mL, 65.93) at 0° C. and stirred at RT for 16 h. Reaction mixture was poured in to ice H₂O (200 mL) and extracted with EtOAc (2×200 mL), dried over anhydrous Na₂SO₄ and evaporated under reduced pressure. The crude residue was purified by Grace Normal Phase with eluent 60% of EtOAc in hexane to afford tert-butyl N²-(tert-butoxycarbonyl)-N⁵-methyl-L-glutaminate (2).

### N²-(tert-butoxycarbonyl)-N⁵-methyl-L-glutamine (3)

To a stirred solution of tert-butyl N²-(tert-butoxycarbonyl)-N⁵-methyl-L-glutaminate (2) (9 g, 28.48 mmol) in methanol (100 mL) and water (10 mL), was added lithium hydroxide (3.41 g, 142.41 mmol) at 0° C. and stirred at room temperature for 16 h. Reaction mixture evaporated under reduced pressure, crude compound acidified with citric acid solution up to pH ˜4, and extracted with ethyl acetate (2×200 mL), dried over sodium sulfate, concentrated under reduced pressure to afford crude compound N²-(tert-butoxycarbonyl)-N⁵-methyl-L-glutamine (3).

### ethyl N²-(tert-butoxycarbonyl)-N⁵-methyl-L-glutaminate (4)

To a stirred solution of N²-(tert-butoxycarbonyl)-N⁵-methyl-L-glutamine (3) (4.2 g, 16.11 mmol) in DMF (25 mL), were added potassium carbonate (3.34 g, 24.23 mmol) and EtI (1.94 mL, 24.23 mmol) at 0° C. then stirred at room temperature for 3 h, then to the reaction mixture added ice water (100 mL) and extracted with EtOAc (2×100 mL). Combined organic layer were washed with NaHCO₃ solution (100 mL), brine solution (100 mL) and dried over anhydrous Na₂SO₄ and concentrated under reduced pressure and purified by Grace normal phase, compound eluted at 70% EtOAc in pet ether to afford pure compound ethyl N²-(tert-butoxycarbonyl)-N⁵-methyl-L-glutaminate (4).

### ethyl N⁵-methyl-L-glutaminate hydrochloride (5)

To a stirred solution of ethyl N²-(tert-butoxycarbonyl)-N⁵-methyl-L-glutaminate (5) (1.1 g, 3.81 mmol) in 1,4-dioxane (10 mL), was added 4N HCl in 1,4-dioxane (10 mL) at 0° C. then reaction mixture was stirred at room temperature for 3 h. After consumption of starting material by TLC, distilled off the solvent to afford crude ethyl N⁵-methyl-L-glutaminate hydrochloride (5).

### ethyl N²-((tert-butoxycarbonyl)-L-leucyl)-N⁵-methyl-L-glutaminate (7)

To a stirred solution of (tert-butoxycarbonyl)-L-leucine (6) (900 mg, 3.89 mmol) in DMF (10 mL), were added EDC.HCl (1.11 g, 5.83 mmol), HOBT (790 mg, 5.83 mmol) and DIPEA (2.15 mL, 11.67 mmol) at 0° C. and stirred for 10 min, then added ethyl N5-methyl-L-glutaminate hydrochloride (5) (958 mg, 4.28 mmol) at 0° C. and stirred at room temperature for 16 h. Reaction mixture was diluted with ice water (25 mL), extracted with EtOAc (2×25 mL), dried over anhydrous Na₂SO₄ and evaporated under reduced pressure. The crude residue was purified by Grace normal phase with eluent of 60% EtOAc in pet ether to afford ethyl N²-((tert-butoxycarbonyl)-L-leucyl)-N⁵-methyl-L-glutaminate (7).

### ethyl N²-(L-leucyl)-N⁵-methyl-L-glutaminate hydrochloride (8)

To a stirred solution of ethyl N²-((tert-butoxycarbonyl)-L-leucyl)-N^{P}-methyl-L-glutaminate (7) (600 mg, 1.49 mmol) in 1,4-dioxane (10 mL), was added 4N HCl in 1,4-dioxane (10 mL) at 0° C. then reaction mixture was stirred at room temperature for 3 h. After consumption of starting material by TLC, distilled off the solvent to afford crude ethyl N²-(L-leucyl)-N⁵-methyl-L-glutaminate hydrochloride (8).

### ethyl N⁵-methyl-N²-(nonanoyl-L-leucyl)-L-glutaminate (9)

To a stirred solution of Nonanoic acid (400 mg, 2.52 mmol) in DMF (5 mL), were added EDC.HCl (726 mg, 3.79 mmol), HOBT (513 mg, 3.79 mmol) and DIPEA (1.4 mL, 7.58 mmol) at 0° C. and stirred for 10 min, then added ethyl N²-(L-leucyl)-N⁵-methyl-L-glutaminate (8) (937 mg, 2.78 mmol) at 0° C. and stirred at room temperature for 16 h. Reaction mixture was diluted with ice water (10 mL), extracted with EtOAc (2×20 mL), dried over anhydrous Na₂SO₄ and evaporated under reduced pressure. The crude residue was purified by column Grace normal phase with eluent 80% EtAOc in pet ether to afford ethyl N⁵-methyl-N²-(nonanoyl-L-leucyl)-L-glutaminate (9).

### N—((S)-1-(((S)-1-hydroxy-5-(methylamino)-5-oxopentan-2-yl)amino)-4-methyl-1-oxopentan-2-yl)nonanamide A110

To a stirred solution of ethyl N⁵-methyl-N²-(nonanoyl-L-leucyl)-L-glutaminate (9) (400 mg, 0.907 mmol) in dry DCM (4 mL), was added 2M LiBH₄ (1.3 mL, 2.72 mmol) at 0° C. and the reaction mixture was stirred at 0° C. for 3 h. After consumption of starting material, the reaction mixture was poured in ice water (10 mL) and extracted with DCM (2×10 mL), combined organic layer was washed with brine, dried over Na₂SO₄ and concentrated, crude compound was purified by Grace normal phase using 100% ethyl acetate as eluent to afford N—((S)-1-(((S)-1-hydroxy-5-(methylamino)-5-oxopentan-2-yl)amino)-4-methyl-1-oxopentan-2-yl)nonanamide A110

TLC system: 100% EtOAc in pet ether Rf: 0.1

LCMS (ESI): m/z 400.30 (M+H)⁺

### N—((S)-1-(((S)-1-chloro-5-(methylamino)-5-oxopentan-2-yl)amino)-4-methyl-1-oxopentan-2-yl)nonanamide A109

To a stirred solution of N—((S)-1-(((S)-1-hydroxy-5-(methylamino)-5-oxopentan-2-yl)amino)-4-methyl-1-oxopentan-2-yl)nonanamide A110 (140 mg, 0.35 mmol) in dry DCM (5 mL) was cooled to 0° C. and added SOCl₂ (0.05 mL, 0.70 mmol) then the reaction mixture was stirred at 0° C. for 2 h. After consumption of starting material, the reaction mixture was poured in to ice water (10 mL) and extracted with DCM (2×10 mL), organic layer was washed with brine solution, dried over Na₂SO₄ and concentrated, Crude compound was purified by prep-HPLC to afford N—((S)-1-(((S)-1-chloro-5-(methylamino)-5-oxopentan-2-yl)amino)-4-methyl-1-oxopentan-2-yl)nonanamide A109.

TLC system: 100% EtOAc in pet ether Rf: 0.5

LCMS (ESI): m/z 418.46 (M+H)⁺

### Synthesis of Compound A1

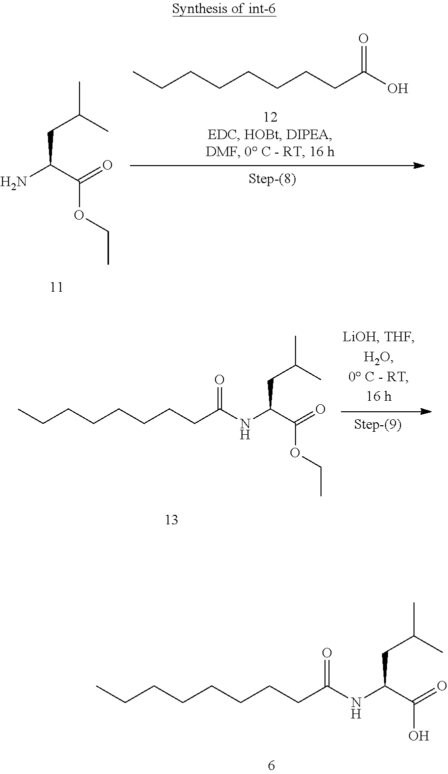

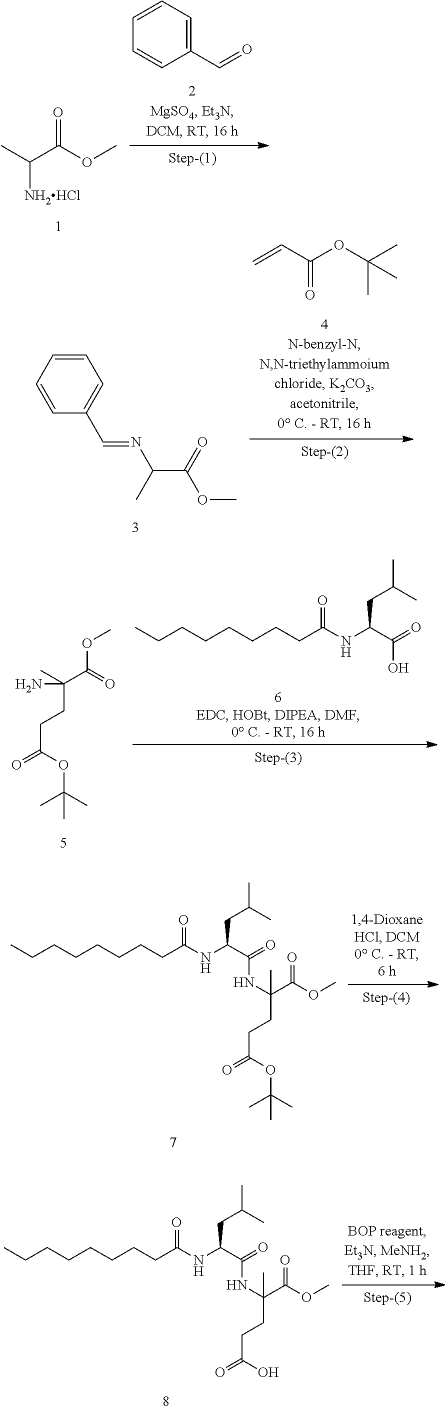

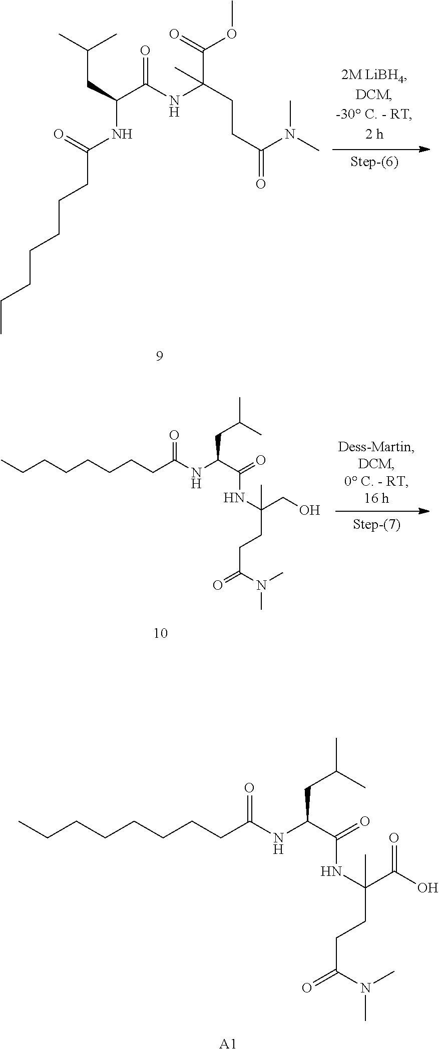

### Methyl (E)-2-(benzylideneamino)propanoate (3)

To a stirred solution of methyl alaninate hydrochloride (1) (2 g, 14.388 mmol) in DCM (30 mL) at 0° C. was added magnesium sulphate (1.2 g, 10.028 mmol), Et₃N (2.4 mL) and benzaldehyde (1.5 g, 14.326 mmol) and raised temperature to RT and stirred for 16 h. Reaction mixture filtered through celite pad and celite bed was washed with DCM. Organic layer washed with water (150 mL), extracted with DCM (2×150 mL), dried over anhydrous Na₂SO₄ and evaporated under reduced pressure to afford compound methyl (E)-2-(benzylideneamino)propanoate (3).

### 5-(tert-butyl) 1-methyl 2-amino-2-methylpentanedioate (5)

To a stirred solution of methyl (E)-2-(benzylideneamino)propanoate (3) (1 g, 4.83 mmol) in acetonitrile (20 mL) at 0° C. was added K₂CO₃ (1.9 mg, 14.49 mmol), N-benzyl-N,N,N-triethyl ammonium chloride (109 mg, 0.483 mmol) sequentially and stirred at room temperature for 16 h. Reaction mixture was diluted with ice water (50 mL) extracted with EtOAc (2×50 mL), dried over Na₂SO₄ and evaporated under reduced pressure. The crude residue was purified by silica column purification to obtained 5-(tert-butyl) 1-methyl 2-amino-2-methylpentanedioate (5).

### Ethyl nonanoyl-L-leucinate (13)

To a stirred solution of nonanoic acid (12) (3.0 g, 18.987 mmol) in DMF (60 mL), were added EDC.HCl (7.16 g, 37.37 mmol), HOBT (5.04 g, 37.37 mmol) and Et₃N (10.5 mL, 75.94 mmol) at 0° C. and stirred 10 min, was added ethyl L-leucinate (11) (4.4 g, 22.78 mmol) at 0° C. and stirred at room temperature for 16 h. Reaction mixture was diluted with ice water (150 mL), extracted with ethyl acetate (2×150 mL), dried over sodium sulfate and evaporated under reduced pressure. The crude residue was purified by column Grace normal phase with eluent 10% EtOAc in pet ether to afford ethyl nonanoyl-L-leucinate (13).

### Nonanoyl-L-leucine (6)

To a stirred solution of ethyl nonanoyl-L-leucinate (13) (240 mg, 0.716 mmol) in THE (2 mL) and water (0.5 mL), was added LiOH (69 mg, 2.86 mmol) at 0° C. and stirred at room temperature for 16 h. Reaction mixture completely distilled under reduced pressure, crude compound acidified with 2N HCl solution up to pH-4, and extracted with EtOAc (2×20 mL), dried over anhydrous Na₂SO₄, concentrated under reduced pressure to afford nonanoyl-L-leucine (6).

### 5-(tert-butyl) 1-methyl 2-methyl-2-((S)-4-methyl-2-nonanamidopentanamido)pentanedioate (7)

To a stirred solution of nonanoyl-L-leucine (6) (250 mg, 0.92 mmol) in DMF (15 mL) was added EDC.HCl 353 mg, 1.84 mmol), HOBT (249 mg, 1.84 mmol) and Et₃N (0.5 mL, 3.66 mmol) at 0° C. and stirred for 10 min, then added 5-(tert-butyl) 1-methyl 2-amino-2-methylpentanedioate (5) (250 mg, 0.92 mmol) at 0° C. and stirred at room temperature for 16 h. Reaction mixture was diluted with ice water (50 mL), extracted with EtOAc (2×50 mL), dried over anhydrous Na₂SO₄ and evaporated under reduced pressure. The crude residue was purified by column Grace normal phase using 20% ethyl acetate in pet ether to as eluent afford 5-(tert-butyl) 1-methyl 2-methyl-2-((S)-4-methyl-2-nonanamidopentanamido)pentanedioate (7).

### 5-methoxy-4-methyl-4-((S)-4-methyl-2-nonanamidopentanamido)-5-oxopentanoic acid (8)

To a stirred solution of 5-(tert-butyl) 1-methyl 2-methyl-2-((S)-4-methyl-2-nonanamidopentanamido)pentanedioate (7) (200 mg, 0.41 mmol) in DCM (2 mL) at 0° C. was added 4N dioxane HCl (0.4 mL, 2 v (w/w)) and stirred at room temperature for 6 h. Solvent was distilled off under reduced pressure to afford crude residue. Crude was basified with aq.NaHCO₃ (10 mL) and extracted with EtOAc (2×30 mL), dried over anhydrous Na₂SO₄ and evaporated under reduced pressure to obtained 5-(tert-butyl) 1-methyl 2-amino-2-methylpentanedioate (8).

### Methyl 5-(dimethylamino)-2-methyl-2-((S)-4-methyl-2-nonanamidopentanamido)-5-oxopentanoate (9)

To a stirred solution of 5-(tert-butyl) 1-methyl 2-amino-2-methylpentanedioate (8) (2 g, 4.55 mmol) in THE (24 mL) was added Bop reagent (2.4 g, 5.46 mmol), Et₃N (1.9 mL, 13.66 mmol) and 2M Me₂NH (4.5 mL) (9.11 mmol) at RT and stirred at same temperature for 1 h. Reaction mixture was diluted with ice water (50 mL), extracted with EtOAc (2×50 mL), dried over anhydrous Na₂SO₄ and evaporated under reduced pressure. The crude residue was purified by Grace normal phase using 50% EtOAc in pet ether to afford methyl 5-(dimethylamino)-2-methyl-2-((S)-4-methyl-2-nonanamidopentanamido)-5-oxopentanoate (9).

### N-((2S)-1-((5-(dimethylamino)-1-hydroxy-2-methyl-5-oxopentan-2-yl)amino)-4-methyl-1-oxopentan-2-yl)nonanamide (10)

To a stirred solution of methyl 5-(dimethylamino)-2-methyl-2-((S)-4-methyl-2-nonanamidopentanamido)-5-oxopentanoate (9) (1 g, 2.19 mmol) in DCM (10 mL) at −30° C. added lithium borohydride (2 M in THF) (1.6 mL, 3.29 mmol) and stirred at same temperature for 2 h. Reaction mixture quenched with 2N HCl and extracted with EtOAc (2×50 mL), dried over anhydrous Na₂SO₄ and evaporated under reduced pressure. The crude residue was purified by Grace normal phase using 5% MeOH/DCM to afford N-((2S)-1-((5-(dimethylamino)-1-hydroxy-2-methyl-5-oxopentan-2-yl)amino)-4-methyl-1-oxopentan-2-yl)nonanamide (10).

### N-((2S)-1-((5-(dimethylamino)-2-methyl-1,5-dioxopentan-2-yl)amino)-4-methyl-1-oxopentan-2-yl)nonanamide A1

To a stirred solution of N-((2S)-1-((5-(dimethylamino)-1-hydroxy-2-methyl-5-oxopentan-2-yl)amino)-4-methyl-1-oxopentan-2-yl)nonanamide (10) (200 mg, 0.468 mmol) in DCM (2 mL) was added Dess-Martin periodinane (297 mg, 0.702 mmol) and stirred at room temperature for 16 h. Reaction mixture was diluted with ice water (20 mL), extracted with EtOAc (2×30 mL), dried over anhydrous Na₂SO₄ and evaporated under reduced pressure. The crude residue was purified by silica column purification to obtained N-((2S)-1-((5-(dimethylamino)-2-methyl-1,5-dioxopentan-2-yl)amino)-4-methyl-1-oxopentan-2-yl)nonanamide A1.

TLC system: 5% Methanol in dichloromethane; Rf: 0.5

LCMS (ESI): m/z 426.33 (M+H)⁺

### Synthesis of Compound A111

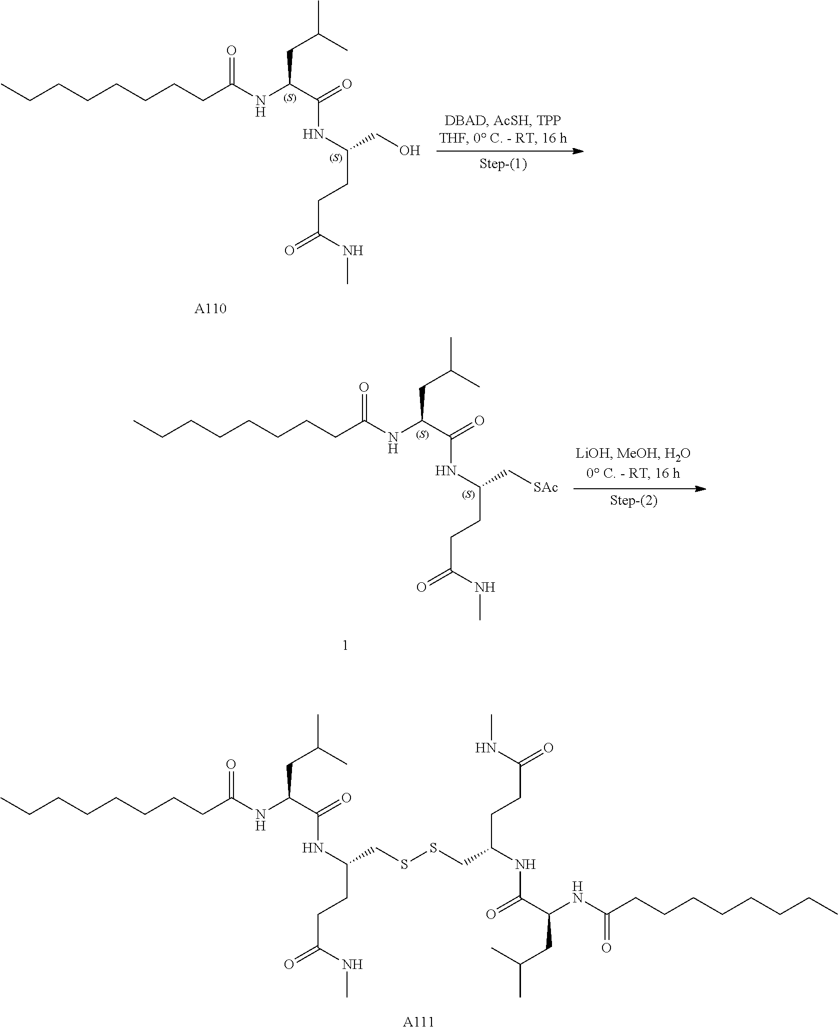

### S—((S)-2-((S)-4-methyl-2-nonanamidopentanamido)-5-(methylamino)-5-oxopentyl) ethanethioate (1)

To a stirred solution of DBAD (576 mg, 2.50 mmol), TPP (656 mg, 2.50 mmol) in dry THE (10 mL) at 0° C. was added thioacetic acid (0.101 mg, 1.37 mmol) and stirred for 5 min. then added N—((S)-1-(((S)-1-hydroxy-5-(methylamino)-5-oxopentan-2-yl)amino)-4-methyl-1-oxopentan-2-yl)nonanamide A110 (500 mg, 1.25 mmol) at 0° C. and stirred at RT for 16 h. After consumption of starting material, the reaction mixture was poured in ice water (10 mL) and extracted with EtOAc (2×10 mL). Organic layer was washed with brine solution, dried over anhydrous Na₂SO₄ and evaporated under vacuum. Crude compound was purified by column chromatography using 80% EtOAc in pet ether as eluent to afford S—((S)-2-((S)-4-methyl-2-nonanamidopentanamido)-5-(methylamino)-5-oxopentyl) ethanethioate (1)

### N,N′-((2S,2'S)-(((2S,2'S)-disulfanediylbis(5-(methylamino)-5-oxopentane-1,2-diyl))bis(azanediyl))bis(4-methyl-1-oxopentane-1,2-diyl))dinonanamide A111

To a stirred solution of S—((S)-2-((S)-4-methyl-2-nonanamidopentanamido)-5-(methylamino)-5-oxopentyl) ethanethioate (1) (600 mg, 1.31 mmol) in MeOH (10 mL) and H₂O (2 mL) was added LiOH (63 mg, 2.62 mmol) at 0° C. and stirred at room temperature for 16 h. Reaction mixture was distilled off under reduced pressure and crude residue was acidified to pH ˜4 with citric acid solution and extracted with EtOAc (2×20 mL), dried over Na₂SO₄, concentrated under reduced pressure. It was purified by prep HPLC to afford N,N′-((2S,2'S)-(((2S,2'S)-disulfanediylbis(5-(methylamino)-5-oxopentane-1,2-diyl))bis(azanediyl))bis(4-methyl-1-oxopentane-1,2-diyl))dinonanamide A111.

TLC system: 10% methanol in DCM; Rf: 0.2

LCMS (ESI): m/z 829.5 (M+H)⁺

### Synthesis of Compound A112

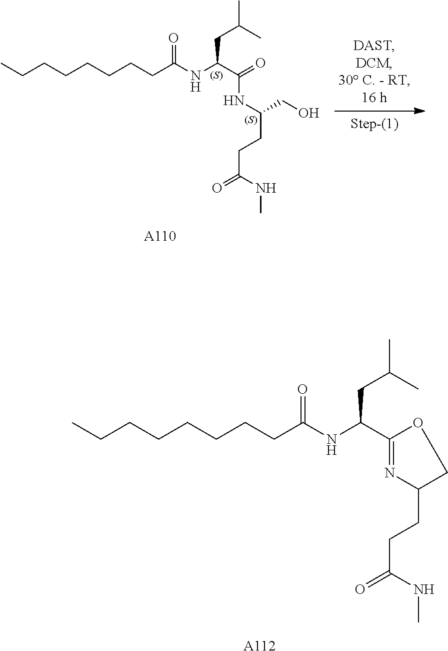

To a stirred solution of N—((S)-1-(((S)-1-hydroxy-5-(methylamino)-5-oxopentan-2-yl)amino)-4-methyl-1-oxopentan-2-yl)nonanamide A110 (250 mg, 0.63 mmol) in dry DCM (25 mL), was added diethylaminosulfur trifluoride (0.12 mL, 0.93 mmol) at −20° C. and the reaction mixture was stirred at 0° C. for 2 h. After consumption of starting material, the reaction mixture was poured in ice water (10 mL) and extracted with DCM (2×20 mL). Organic layer was washed with brine solution, dried over Na₂SO₄ and concentrated, crude compound was purified by prep HPLC to afford N-((1 S)-3-methyl-1-(4-(3-(methylamino)-3-oxopropyl)-4,5-dihydrooxazol-2-yl)butyl)nonanamide A112.

TLC system: 10% methanol in DCM; Rf: 0.5

LCMS (ESI): m/z 382.41 (M+H)⁺

### Synthesis of Compound A2

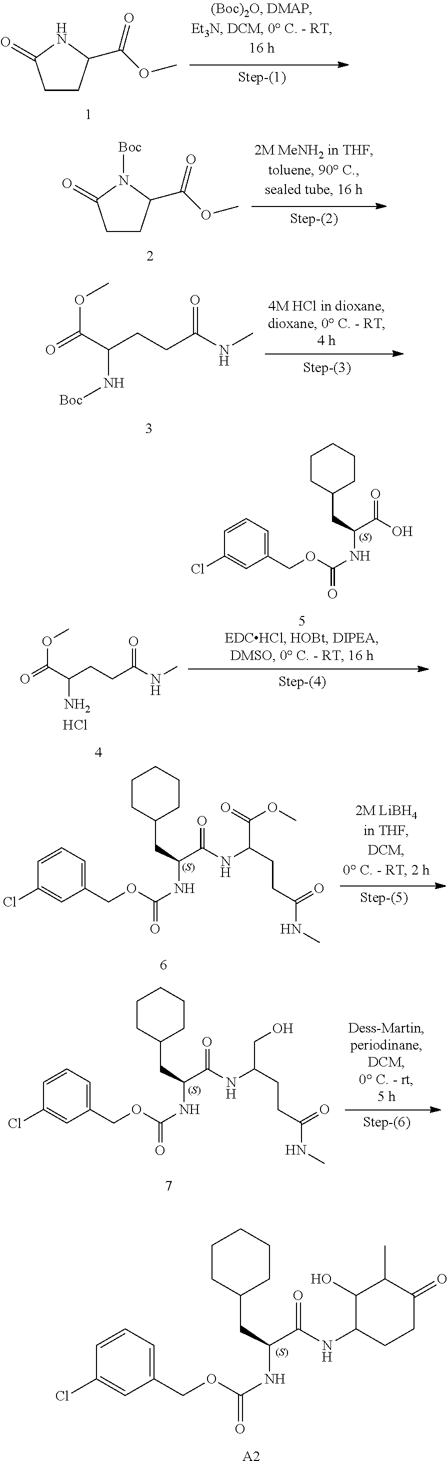

### 1-(tert-butyl) 2-methyl 5-oxopyrrolidine-1,2-dicarboxylate (2)

To a stirred solution of methyl 5-oxopyrrolidine-2-carboxylate (1) (10 g, 69.93 mmol) in DCM (100 mL) at 0° C. was added Et₃N (30 mL, 209.79 mmol) and DMAP (1.7 g, 13.98 mmol) and stirred for 10 min, then added (Boc)₂O (30.4 mL, 139.86 mmol) at 0° C. and stirred at RT for 16 h. Reaction mixture was poured in to ice cold H₂O (200 mL) and extracted with DCM (2×200 mL), dried over anhydrous Na₂SO₄ and evaporated under reduced pressure. The crude residue was purified by Grace normal phase eluent with 10% EtOAc in hexane to afford 1-(tert-butyl) 2-methyl 5-oxopyrrolidine-1,2-dicarboxylate (2).

### Methyl N²-(tert-butoxycarbonyl)-N⁵-methylglutaminate (3)

To a stirred solution of 1-(tert-butyl) 2-methyl 5-oxopyrrolidine-1,2-dicarboxylate (2) (2 g, 8.23 mmol) in toluene (20 mL) and 2M methyl amine (12.3 mL) taken in a sealed tube and heated to 90° C. for 16 h. Reaction mixture was poured in to ice H₂O (20 mL) and extracted with EtOAc (2×20 mL), dried over anhydrous Na₂SO₄ and evaporated under reduced pressure. The crude residue was purified by Grace normal phase eluted with 2% MeOH in DCM to afford methyl N²-(tert-butoxycarbonyl)-N⁵-methylglutaminate (3).

### Methyl N⁵-methylglutaminate hydrochloride (4)

To a stirred solution of methyl N²-(tert-butoxycarbonyl)-N⁵-methylglutaminate (3) (2.2 g, 8.02 mmol) in 1,4-dioxane (20 mL) was added 4N HCl in 1,4-dioxane (4 mL) at 0° C. and the reaction mixture was stirred at room temperature for 4 h. After consumption of starting material by TLC, dioxane solvent distilled off to afford crude methyl N⁵-methylglutaminate hydrochloride (4).

### Methyl N²—((S)-2-((((3-chlorobenzyl)oxy)carbonyl)amino)-3-cyclohexylpropanoyl)-N⁵-methylglutaminate (6)

To a stirred solution of (S)-2-((((3-chlorobenzyl)oxy)carbonyl)amino)-3-cyclohexylpropanoic acid (5) (250 mg, 0.73 mmol) (synthesis reported in Compd-17 final report) in DMSO (5 mL), were added EDC.HCl (211 mg, 1.1061 mmol), HOBT (150 mg, 1.1061 mmol) and DIPEA (0.4 mL, 2.2123 mmol) at 0° C. and stirred 10 min, was added methyl N⁵-methylglutaminate hydrochloride (4) (141 mg, 0.8112 mmol) in 2 mL DMSO at 0° C. and stirred at room temperature for 16 h. Reaction mixture was diluted with ice water (25 mL), extracted with ethyl acetate (2×25 mL), dried over sodium sulfate and evaporated under reduced pressure. The crude residue was purified by column Grace (RP) with 0.1% F.A in water acetonitrile to afford methyl N²—((S)-2-((((3-chlorobenzyl) oxy) carbonyl) amino)-3-cyclohexylpropanoyl)-N⁵-methylglutaminate (6).

### 3-chlorobenzyl ((2S)-3-cyclohexyl-1-((1-hydroxy-5-(methylamino)-5-oxopentan-2-yl)amino)-1-oxopropan-2-yl)carbamate (7)

To a stirred solution of methyl N²—((S)-2-((((3-chlorobenzyl) oxy) carbonyl) amino)-3-cyclohexylpropanoyl)-N⁵-methylglutaminate (6) (150 mg, 0.303 mmol) in dry DCM (5 mL), was added 2M LiBH₄ (0.3 mL, 0.60 mmol) at 0° C. and the reaction mixture was stirred at 0° C. for 3 h. After consumption of starting material, the reaction mixture was poured in ice water (10 mL) and extracted with DCM (2×10 mL), Organic layer was washed with brine solution, dried over Na₂SO₄ and concentrated, crude compound was purified by Grace (RP) afford 3-chlorobenzyl ((2S)-3-cyclohexyl-1-((1-hydroxy-5-(methylamino)-5-oxopentan-2-yl)amino)-1-oxopropan-2-yl)carbamate (7).

### 3-chlorobenzyl ((2S)-3-cyclohexyl-1-((2-hydroxy-1-methyl-6-oxopiperidin-3-yl) amino)-1-oxopropan-2-yl)carbamate A2

3-chlorobenzyl ((2S)-3-cyclohexyl-1-((1-hydroxy-5-(methylamino)-5-oxopentan-2-yl) amino)-1-oxopropan-2-yl) carbamate (7) (100 mg, 0.214 mmol) was dissolved in DCM (10 mL) under a nitrogen atmosphere and cooled to 0° C. Dess-Martin periodinane reagent (181 mg, 0.37 mmol) was added to the reaction mixture. The ice bath was removed and the reaction mixture was stirred at room temperature for 3 h (monitoring by TLC indicated complete disappearance of the starting material). A solution of 10% aqueous sodium thiosulfate (20 mL) was added, and the solution was stirred for another 15 min. The aqueous layer was removed, and the organic layer was washed with 10% aqueous sodium thiosulfate (20 mL), followed by saturated aqueous sodium bicarbonate (2×20 mL), water (2×20 mL), and brine (20 mL). The organic layer was dried over anhydrous sodium sulfate and concentrated. The yellow residue was purified by Prep HPLC to afforded 3-chlorobenzyl ((2S)-3-cyclohexyl-1-((2-hydroxy-1-methyl-6-oxopiperidin-3-yl) amino)-1-oxopropan-2-yl) carbamate A2.

TLC system: 10% methanol in DCM; Rf: 0.25

LCMS (ESI): m/z 466.25 (M+H)⁺

### Synthesis of Compound A3

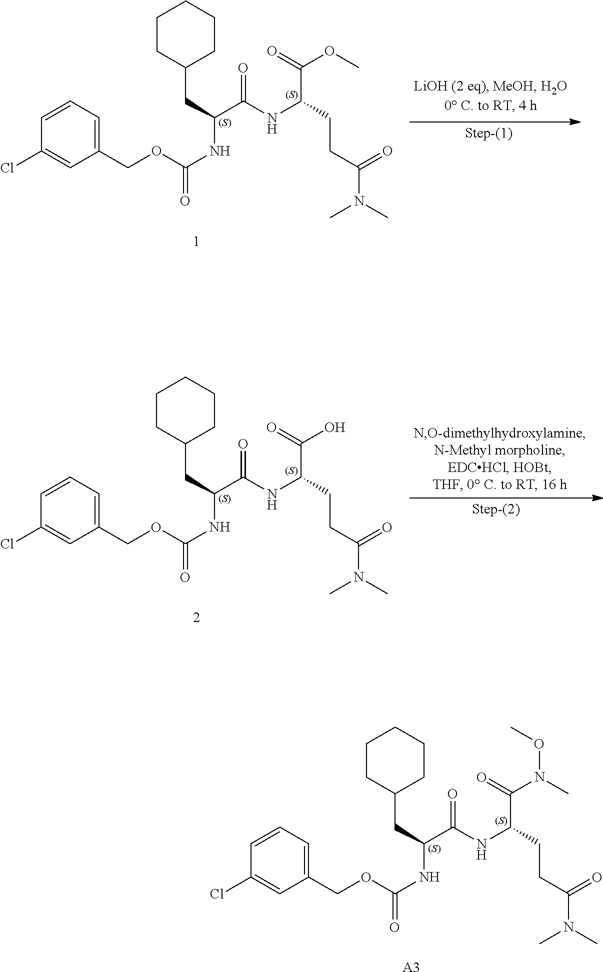

### N2-((S)-2-((((3-chlorobenzyl)oxy)carbonyl)amino)-3-cyclohexylpropanoyl)-N5,N5-dimethyl-L-glutamine (2)

To a stirred solution of methyl N2-((S)-2-((((3-chlorobenzyl)oxy)carbonyl)amino)-3-cyclohexylpropanoyl)-N5,N5-dimethyl-L-glutaminate (1) (500 mg, 0.982 mmol) in methanol (15 mL), water (5 mL) was added LiOH (47 mg, 1.964 mmol) at 0° C. and the reaction mixture was stirred at RT for 2 h. After consumption of starting material, the reaction mixture concentrated under reduced pressure to afford crude product; this crude was acidified with 1N HCl and extracted with ethyl acetate (2×100 mL). Organic layer was washed with brine solution, dried over Na₂SO₄ and concentrated to afford N2-((S)-2-((((3-chlorobenzyl) oxy) carbonyl) amino)-3-cyclohexylpropanoyl)-N5, N5-dimethyl-L-glutaminedimethyl-L-glutamine (2).

### 3-Chlorobenzyl ((S)-3-cyclohexyl-1-(((S)-5-(dimethylamino)-1-(methoxy (methyl) amino)-1,5-dioxopentan-2-yl)amino)-1-oxopropan-2-yl)carbamate Compound A3

To a stirred solution of N2-((S)-2-((((3-chlorobenzyl) oxy) carbonyl) amino)-3-cyclohexylpropanoyl)-N5, N5-dimethyl-L-glutaminedimethyl-L-glutamine (190 mg, 0.384 mmol) in THE (5 mL), DCM (5 mL) was added EDC.HCl (88 mg, 0.461 mmol), HOBt (62 mg, 0.461 mmol), N-methyl morpholine (0.066 mL, 0.461 mmol) followed by N,O-dimethyl hydroxylamine (44 mg, 0.461 mmol) at 0° C. then stirred at RT for 16 h. The progress of the reaction was monitored by TLC. After consumption of starting material the reaction mixture was diluted with DCM and washed with water (2×30 mL). Organic layer was washed with brine solution, dried over anhydrous Na₂SO₄ and concentrated to afford crude product; this crude was purified by normal phase chromatography using 3% methanol in dichloromethane to afford pure 3-chlorobenzyl ((S)-3-cyclohexyl-1-(((S)-5-(dimethylamino)-1-(methoxy(methyl)amino)-1,5-dioxopentan-2-yl)amino)-1-oxopropan-2-yl)carbamate A3.

TLC system: 10% Methanol in DCM; Rf: 0.4

LCMS (ESI): m/z 539.32 (M+H)⁺

### Synthesis of Compound A4

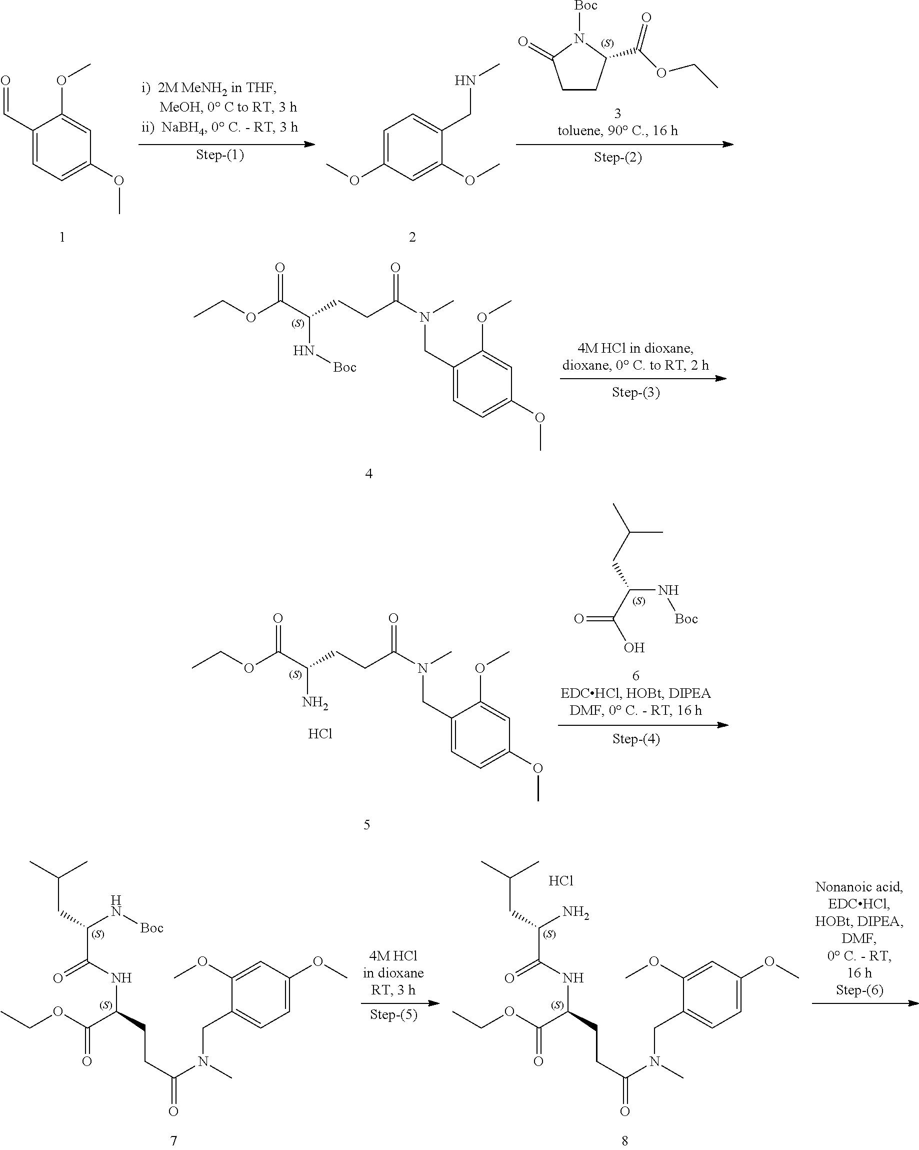

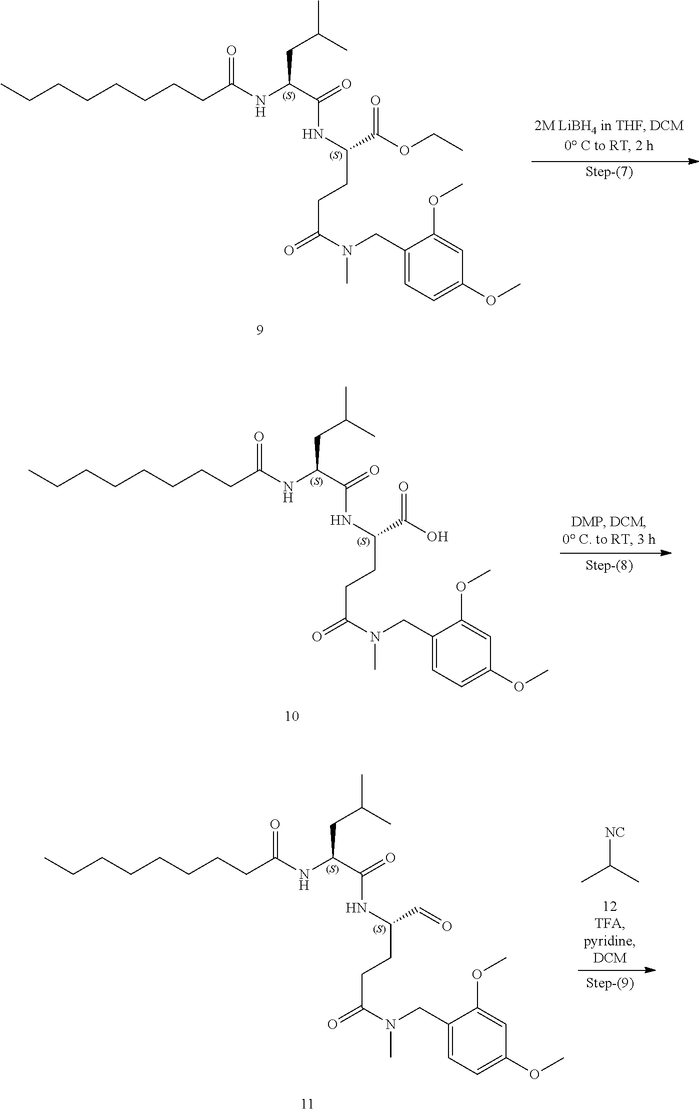

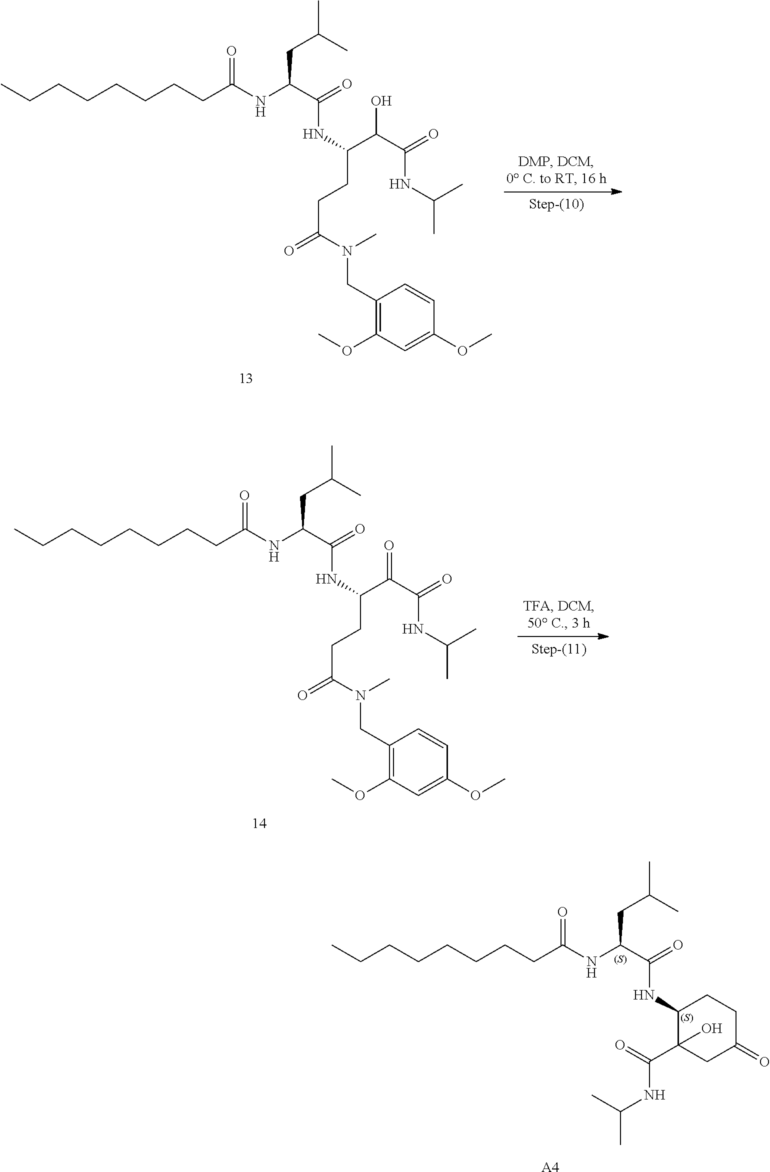

### 1-(2,4-Dimethoxyphenyl)-N-methylmethanamine (2)

To a stirred solution of 2,4-dimethoxybenzaldehyde (1) (10 g, 60.24 mmol) in MeOH (100 mL) was cooled to 0° C. and added 2M MeNH₂ in THE (30 mL, 60.24 mmol). Then reaction mixture allowed to stir at RT for 1 h. Then added NaBH₄ (3.3 g, 90.36 mmol) slowly portion wise at 0° C. and the reaction mixture was stirred at RT for 3 h. After consumption of starting material, the reaction mixture acidified with 2N HCl (100 mL) and total solvent was evaporated. Then reaction mixture was basified with 2 N NaOH solution and adjusted pH up to 13 and extracted with EtOAc (2×400 mL). Organic layer was washed with brine solution, dried over Na₂SO₄ and concentrated to afforded pure compound of 1-(2,4-dimethoxyphenyl)-N-methylmethanamine (2).

### Ethyl N²-(tert-butoxycarbonyl)-N⁵-(2,4-dimethoxybenzyl)-N⁵-methyl-L-glutaminate (4)

To a stirred solution of 1-(2,4-dimethoxyphenyl)-N-methylmethanamine (2) (1 g, 3.89 mmol) in toluene (10 mL) was added 1-(tert-butyl) 2-ethyl (S)-5-oxopyrrolidine-1,2-dicarboxylate (0.7 g, 3.89 mmol) at RT then reaction mixture was heated to 90° C. for 16 h. The progress of the reaction was monitored by TLC & LC-MS. After consumption of starting material, the reaction mixture was cooled to RT and the volatiles were evaporated under reduced pressure to obtain crude compound, crude was purified through silica gel (100-200 mesh) column chromatography by eluting with 40% EtOAc in pet ether to afford Ethyl N²-(tert-butoxycarbonyl)-N⁵-(2,4-dimethoxybenzyl)-N⁵-methyl-L-glutaminate (4).

### Ethyl N⁵-(2,4-dimethoxybenzyl)-N⁵-methyl-L-glutaminate (5)

To a stirred solution of Ethyl N²-(tert-butoxycarbonyl)-N⁵-(2,4-dimethoxybenzyl)-N⁵-methyl-L-glutaminate (4) (9 g, 20.54 mmol) in 1,4-dioxane (90 mL) was added 4M HCl in dioxane (30.8 mL) at 0° C. The mixture was allowed to RT and stirred for 2 h. After consumption of starting material, solvent was evaporated to afford crude salt. It was triturated with diethyl ether to afford Ethyl N⁵-(2,4-dimethoxybenzyl)-N⁵-methyl-L-glutaminate (5).

### Ethyl N²-((tert-butoxycarbonyl)-L-leucyl)-N⁵-(2,4-dimethoxybenzyl)-N⁵-methyl-L-glutaminate (7)

To a stirred solution of (tert-butoxycarbonyl)-L-leucine (6) (5.4 g, 23.66 mmol)) in dry DMF (80 mL) at 0° C. was added EDC.HCl (4.5 g, 28.39 mmol), HOBt (3.8 g, 28.39 mmol) and DIPEA (13 mL, 70.98 mmol) and stirred for 15 minutes at RT then added Ethyl N5-(2,4-dimethoxybenzyl)-N5-methyl-L-glutaminate (5) (8.0 g, 23.66 mmol) at 0° C. then reaction mixture was allowed to stirred at room temperature for 16 h. After consumption of starting material, the reaction mixture was quenched with ice water (150 mL) and extracted with EtOAc (2×500 mL). Combined organic layer were washed with brine solution, dried over anhydrous Na₂SO₄, filtered and evaporated under reduced pressure. Crude was purified through silica gel (100-200) column chromatography by eluting with 7% MeOH in DCM to afford Ethyl N²-((tert-butoxycarbonyl)-L-leucyl)-N⁵-(2,4-dimethoxybenzyl)-N⁵-methyl-L-glutaminate (7).

### Ethyl N²-(L-leucyl)-N⁵-(2,4-dimethoxybenzyl)-N⁵-methyl-L-glutaminate (8)

To a stirred solution of Ethyl N²-((tert-butoxycarbonyl)-L-leucyl)-N⁵-(2,4-dimethoxybenzyl)-N⁵-methyl-L-glutaminate (7) (36.29 mmol) in 1,4-dioxane (100 mL) was added 4M HCl in dioxane (60 mL) at 0° C. The mixture was allowed to RT and stirred for 2 h. After consumption of starting material, solvent was evaporated to afford crude salt. It was triturated with pentene to afforded Ethyl N²-(L-leucyl)-N⁵-(2,4-dimethoxybenzyl)-N⁵-methyl-L-glutaminate (8).

### Ethyl N⁵-(2,4-dimethoxybenzyl)-N⁵-methyl-N²-(nonanoyl-L-leucyl)-L-glutaminate (9)

To a stirred solution of Nonanoic acid (3.4 g, 22.17 mmol) in DMF (100 mL) was added EDC.HCl (5.08 g, 26.62 mmol), HOBt (3.59 g, 26.62 mmol), and DIPEA (12.2 mL, 67.86 mmol) at 0° C. then stirred for 15 min and added Ethyl N²-(L-leucyl)-N⁵-(2,4-dimethoxybenzyl)-N⁵-methyl-L-glutaminate (8) (10 g, 22.17 mmol) at 0° C. Reaction mixture was stirred at room temperature for 16 h. The progress of the reaction was monitored by TLC. After consumption of starting material, the reaction mixture was quenched with water (100 mL) and extracted with EtOAc (2×500 mL). Combined organic layer was washed with brine solution, dried over anhydrous Na₂SO₄ and concentrated to get crude; Crude was purified through silica gel (100-200 mesh) column chromatography by eluting with 5% MeOH in DCM to afforded Ethyl N⁵-(2,4-dimethoxybenzyl)-N⁵-methyl-N²-(nonanoyl-L-leucyl)-L-glutaminate.

### N—((S)-1-(((S)-5-((2,4-dimethoxybenzyl)(methyl)amino)-1-hydroxy-5-oxopentan-2-yl)amino)-4-methyl-1-oxopentan-2-yl)nonanamide (10)

To a stirred solution of Ethyl N⁵-(2,4-dimethoxybenzyl)-N⁵-methyl-N²-(nonanoyl-L-leucyl)-L-glutaminate (310 mg, 0.52 mmol) in DCM (5 mL) at 0° C., was added 2M LiBH₄ in THE (0.4 mL, 0.78 mmol) and allowed to room temperature stirred for 2 h. The progress of the reaction was monitored by TLC. After consumption of starting material, reaction mixture was quenched with NH₄Cl solution and extracted with EtOAc (2×50 mL), dried over anhydrous Na₂SO₄ filtered and solvent was evaporated under reduced pressure. Crude was purified through silica gel (100-200 mesh) column chromatography by eluting with 5% MeOH in DCM to afford N—((S)-1-(((S)-5-((2,4-dimethoxybenzyl)(methyl)amino)-1-hydroxy-5-oxopentan-2-yl)amino)-4-methyl-1-oxopentan-2-yl)nonanamide.

### N—((S)-1-(((S)-5-((2,4-dimethoxybenzyl)(methyl)amino)-1,5-dioxopentan-2-yl)amino)-4-methyl-1-oxopentan-2-yl)nonanamide (11)

To a stirred solution of (N—((S)-1-(((S)-5-((2,4-dimethoxybenzyl)(methyl)amino)-1-hydroxy-5-oxopentan-2-yl)amino)-4-methyl-1-oxopentan-2-yl)nonanamide (10) (500 mg, 0.91 mmol) in DCM (5 mL) at 0° C. was added Dess-Martin periodinane (772 mg, 1.82 mmol) and reaction mass was allowed to room temperature stirred for 2 h. The progress of the reaction was monitored by TLC. After consumption of starting material, Reaction mixture was quenched with Hypo solution and NaHCO₃ solution then extracted with ethyl acetate (2×50 mL), dried over sodium sulfate filtered and evaporated under reduced pressure. The crude residue was triturated with di ethyl ether to afford N—((S)-1-(((S)-5-((2,4-dimethoxybenzyl)(methyl)amino)-1,5-dioxopentan-2-yl)amino)-4-methyl-1-oxopentan-2-yl)nonanamide (11).

### (3S)—N6-(2,4-dimethoxybenzyl)-2-hydroxy-N¹-isopropyl-N⁶-methyl-3-((S)-4-methyl-2-nonanamidopentanamido)hexanediamide (13)

To a stirred solution of N—((S)-1-(((S)-5-((2,4-dimethoxybenzyl)(methyl)amino)-1,5-dioxopentan-2-yl)amino)-4-methyl-1-oxopentan-2-yl)nonanamide A4 (2 g, 3.65 mmol) in DCM (20 mL) were added isopropyl isocyanide (11) (500 mg, 7.3 mmol), pyridine (1.2 mL, 14.6 mmol) followed by TFA (0.6 mL, 7.306 mmol) at 0° C. and stirred at RT for 16 h. Reaction mixture was diluted with DCM and washed with sat. NaHCO₃ solution (3×30 mL) followed by brine solution (1×30 mL). Organic layer was dried over sodium sulfate and concentrated to get crude product; this crude was purified by Grace normal phase chromatography by eluting 5% methanol in dichloromethane to afford (3S)—N⁶-(2,4-dimethoxybenzyl)-2-hydroxy-N1-isopropyl-N6-methyl-3-((S)-4-methyl-2-nonanamidopentanamido)hexanediamide (12).

### (S)—N⁶-(2, 4-Dimethoxybenzyl)-N¹-isopropyl-N⁶-methyl-3-((S)-4-methyl-2-nonanamidopentanamido)-2-oxohexanediamide (14)

To a stirred solution of (3S)—N⁶-(2,4-dimethoxybenzyl)-2-hydroxy-N+-isopropyl-N⁶-methyl-3-((S)-4-methyl-2-nonanamidopentanamido)hexanediamide (13) (580 mg, 0.914 mmol) was dissolved in DCM (15 mL) was added Dess-Martin periodinane (1.55 g, 3.65 mmol) at 0° C. and stirred at RT for 16 h. Reaction mixture was diluted with DCM and washed with sat hypo solution (3×30 mL) followed by sat NaHCO₃ solution (3×30 mL). Organic layer was dried over sodium sulfate and concentrated to get crude; this crude compound was purified by normal phase chromatography with eluting 6% methanol in dichloromethane to afford (S)-N6-(2, 4-dimethoxybenzyl)-N¹-isopropyl-N⁶-methyl-3-((S)-4-methyl-2-nonanamidopentanamido)-2-oxohexanediamide (14).

### (3S)-2-hydroxy-N-isopropyl-1-methyl-3-((S)-4-methyl-2-nonanamidopentanamido)-6-oxopiperidine-2-carboxamide A4

To a stirred solution of (S)-N6-(2,4-dimethoxybenzyl)-N¹-isopropyl-N⁶-methyl-3-((S)-4-methyl-2-nonanamidopentanamido)-2-oxohexanediamide (14) (260 mg, 0.411 mmol) in DCM (20 mL) was added TFA (0.34 mL, 4.11 mmol) at RT and stirred at 50° C. for 3 h. Reaction mixture was diluted with DCM (25 mL) and washed with sat. NaHCO₃ solution (3×20 mL). Organic layer was dried over anhydrous Na₂SO₄ and evaporated to get crude residue. Crude compound was purified by prep HPLC to afford (3S)-2-hydroxy-N-isopropyl-1-methyl-3-((S)-4-methyl-2-nonanamidopentanamido)-6-oxopiperidine-2-carboxamide A4.

TLC system: 10% Methanol in DCM; Rf: 0.3

LCMS (ESI): m/z 483.3 (M+H)⁺

### Synthesis of Compound A5

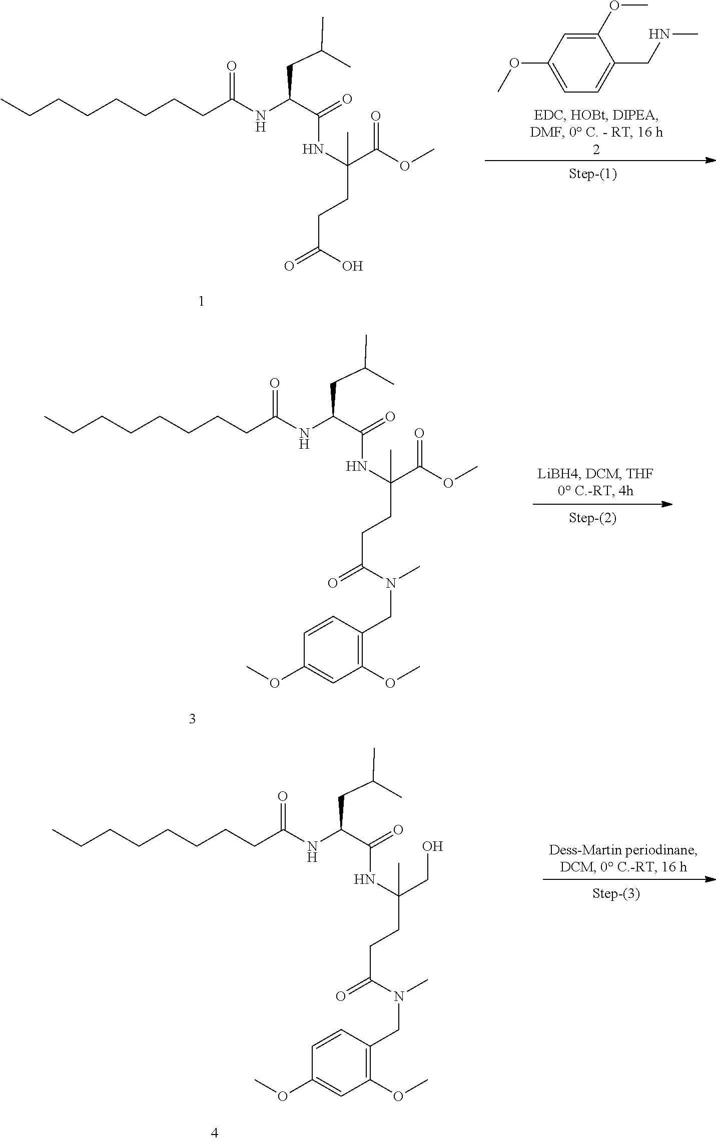

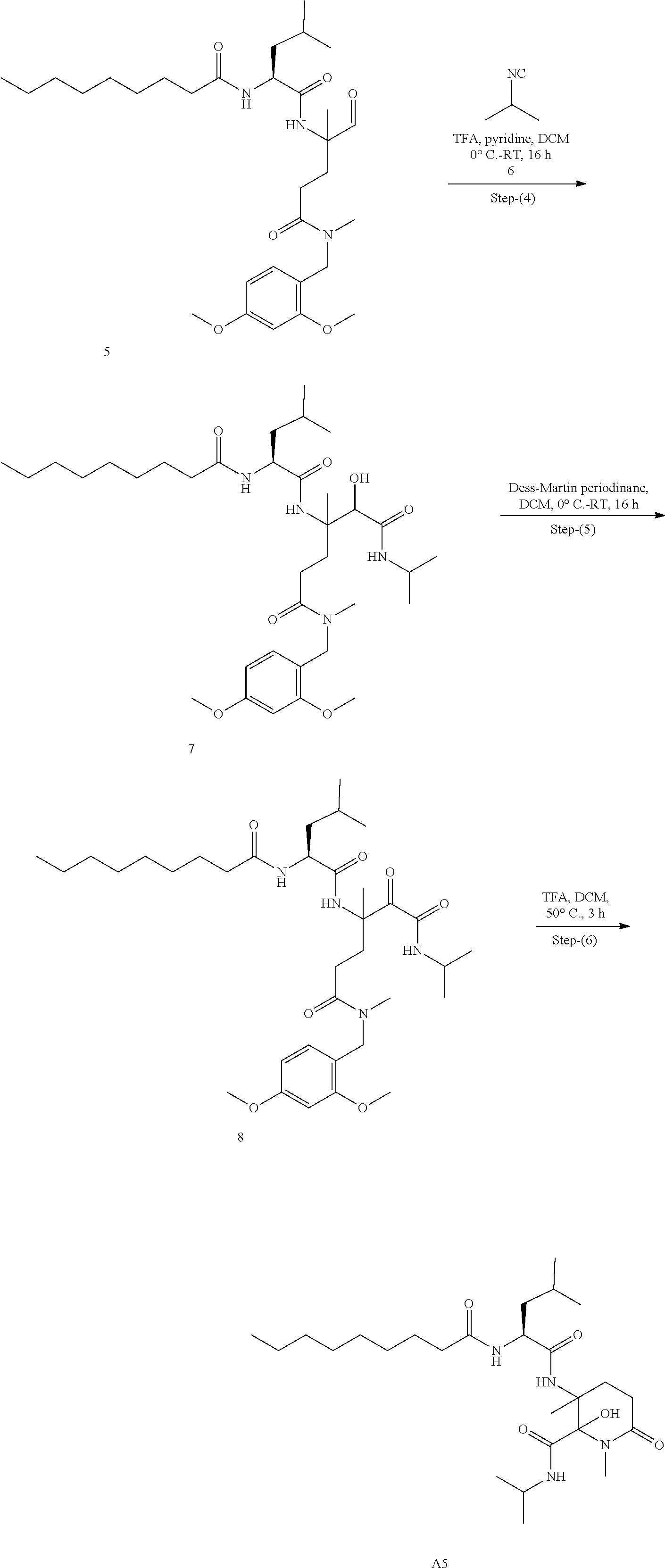

### Methyl 5-((2,4-dimethoxybenzyl)(methyl)amino)-2-methyl-2-((S)-4-methyl-2-nonanamidopentanamido)-5-oxopentanoate (3)

To a stirred solution of 5-methoxy-4-methyl-4-((S)-4-methyl-2-nonanamidopentanamido)-5-oxopentanoic acid (1) (synthesis described in Compound A1) (1.75 g, 3.98 mmol) in DMF (30 mL) at 0° C. was added EDC.HCl (1.14 g, 5.97 mmol), HOBT (807 mg, 5.97 mmol) and Et₃N (1.6 mL, 11.95 mmol) and stirred for 10 min, then added 1-(2,4-dimethoxyphenyl)-N-methylmethanamine (2) (865 mg, 4.78 mmol) at 0° C. and stirred at room temperature for 16 h. Reaction mixture was diluted with ice water (150 mL), extracted with EtOAc (2×150 mL), dried over anhydrous Na₂SO₄ and evaporated under reduced pressure. The crude residue was purified by column Grace normal phase using 60% EtOAc in pet ether to afford methyl 5-((2,4-dimethoxybenzyl)(methyl)amino)-2-methyl-2-((S)-4-methyl-2-nonanamidopentanamido)-5-oxopentanoate (3).

### N-((2S)-1-((5-((2,4-dimethoxybenzyl)(methyl)amino)-1-hydroxy-2-methyl-5-oxopentan-2-yl)amino)-4-methyl-1-oxopentan-2-yl)nonanamide (4)

To a stirred solution of methyl 5-((2,4-dimethoxybenzyl)(methyl)amino)-2-methyl-2-((S)-4-methyl-2-nonanamidopentanamido)-5-oxopentanoate (3) (170 mg, 0.28 mmol) in DCM (4 mL) at 0° C. added lithium borohydride (2M in THF) (0.4 mL, 0.86 mmol) and stirred at same temperature for 4 h. Reaction mixture quenched with 2N HCl and extracted with EtOAc (2×50 mL), dried over anhydrous Na₂SO₄ and evaporated under reduced pressure. The crude residue was purified by Grace normal phase using 5% MeOH/DCM to afford N-((2S)-1-((5-((2,4-dimethoxybenzyl)(methyl)amino)-1-hydroxy-2-methyl-5-oxopentan-2-yl)amino)-4-methyl-1-oxopentan-2-yl)nonanamide (4).

### N-((2S)-1-((5-((2,4-dimethoxybenzyl)(methyl)amino)-2-methyl-1,5-dioxopentan-2-yl)amino)-4-methyl-1-oxopentan-2-yl)nonanamide (5)

To a stirred solution of N-((2S)-1-((5-((2,4-dimethoxybenzyl)(methyl)amino)-1-hydroxy-2-methyl-5-oxopentan-2-yl)amino)-4-methyl-1-oxopentan-2-yl)nonanamide (4) (110 mg, 0.19 mmol) in DCM (2 mL) at 0° C. was added Dess-Martin periodinane (81 mg, 0.39 mmol and stirred at room temperature for 16 h. Reaction mixture was diluted with ice water (20 mL), extracted with EtOAc (2×30 mL), organic layer washed with sat.NaHCO₃ (2×30 mL) and dried over anhydrous Na₂SO₄ and evaporated under reduced pressure. The crude residue was purified by silica gel (100-200 mesh) column purification afforded N-((2S)-1-((5-((2,4-dimethoxybenzyl)(methyl)amino)-2-methyl-1,5-dioxopentan-2-yl)amino)-4-methyl-1-oxopentan-2-yl)nonanamide (5).

### N⁶-(2,4-dimethoxybenzyl)-2-hydroxy-N¹-isopropyl-N⁶,3-dimethyl-3-((S)-4-methyl-2-nonanamidopentanamido)hexanediamide (7)

To a stirred solution of N-((2S)-1-((5-((2,4-dimethoxybenzyl)(methyl)amino)-2-methyl-1,5-dioxopentan-2-yl)amino)-4-methyl-1-oxopentan-2-yl)nonanamide (5) (1.14 g, 2.03 mmol) in DCM (15 mL) at 0° C. was added pyridine (0.4 mL, 8.128 mmol), 2-isocyanopropane (6) (0.3 mL, 4.06 mmol) and TFA (0.3 mL, 4.06 mmol) and stirred at rt for 16 h. Reaction mixture quenched with 1N HCl and extracted with EtOAc (3×50 mL), dried over anhydrous Na₂SO₄, concentrated under reduced pressure to afford compound N⁶-(2,4-dimethoxybenzyl)-2-hydroxy-N¹-isopropyl-N⁶,3-dimethyl-3-((S)-4-methyl-2-nonanamidopentanamido)hexanediamide (7).

### N⁶-(2,4-dimethoxybenzyl)-N¹-isopropyl-N6,3-dimethyl-3-((S)-4-methyl-2-nonanamidopentanamido)-2-oxohexanediamide (8)

To a stirred solution of N⁶-(2,4-dimethoxybenzyl)-2-hydroxy-N¹-isopropyl-N⁶,3-dimethyl-3-((S)-4-methyl-2-nonanamidopentanamido)hexanediamide 7 (400 mg, 0.71 mmol) in DCM (10 mL) at 0° C. was added Dess-Martin periodinane (604 mg, 1.42 mmol) and stirred at room temperature for 16 h. Reaction mixture was diluted with ice water (20 mL), extracted with EtOAc (2×30 mL), organic layer washed with sat.NaHCO₃ (2×30 mL) and dried over anhydrous Na₂SO₄ and evaporated under reduced pressure. The crude residue was purified by silica gel (100-200 mesh) column purification afforded N⁶-(2,4-dimethoxybenzyl)-N¹-isopropyl-N6,3-dimethyl-3-((S)-4-methyl-2-nonanamidopentanamido)-2-oxohexanediamide 8.

### 2-hydroxy-N-isopropyl-1,3-dimethyl-3-((S)-4-methyl-2-nonanamidopentanamido)-6-oxopiperidine-2-carboxamide A5

To a stirred solution of N6-(2,4-dimethoxybenzyl)-N1-isopropyl-N6,3-dimethyl-3-((S)-4-methyl-2-nonanamidopentanamido)-2-oxohexanediamide (8) (500 mg, 0.77 mmol) in DCM (2 mL) was added TFA (0.6 mL, 7.74 mmol) and slowly heated to 50° C. for 6 h. Solvent was evaporated under reduced pressure to afford crude compound. Crude residue was basified with aq. NaHCO₃ (10 mL) and extracted with EtOAc (2×30 mL), dried over anhydrous Na₂SO₄ and evaporated under reduced pressure to obtained 2-hydroxy-N-isopropyl-1,3-dimethyl-3-((S)-4-methyl-2-nonanamidopentanamido)-6-oxopiperidine-2-carboxamide A5.

TLC system: 10% Methanol in dichloromethane; Rf: 0.5

LCMS (ESI): m/z 497.3 (M+H)⁺

### Synthesis of Compound A6

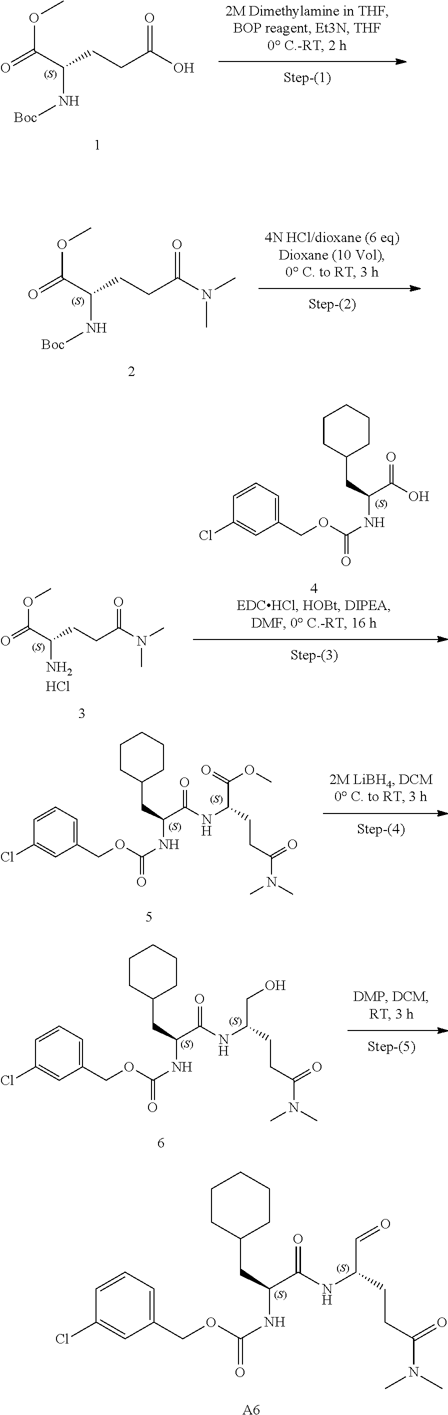

### Methyl N2-(tert-butoxycarbonyl)-N5, N5-dimethyl-L-glutaminate (2)

To a stirred solution of (S)-4-((tert-butoxycarbonyl)amino)-5-methoxy-5-oxopentanoic acid (1) (5 g, 19.15 mmol) in THE (50 mL) cooled to 0° C. and added BOP reagent (10.16 g, 22.18 mmol), 2M dimethylamine in THE (19 mL, 38.31 mmol) followed by Et₃N (3.86 mL, 38.31 mmol) and the reaction mixture was stirred at RT for 2 h. After consumption of starting material, the reaction mixture quenched with water (30 mL) and extracted with ethyl acetate (2×100 mL). Organic layer was washed with brine solution, dried over Na₂SO₄ and concentrated to get crude. Crude compound was purified by column chromatography to afford pure compound methyl N2-(tert-butoxycarbonyl)-N5, N5-dimethyl-L-glutaminate 2.

### Methyl N5, N5-dimethyl-L-glutaminate (3)

To a stirred solution of methyl N2-(tert-butoxycarbonyl)-N5,N5-dimethyl-L-glutaminate (2) (3 g, 10.38 mmol) in 1,4-dioxane (15 mL) was added 4 M HCl in dioxane (15 mL, 62.28 mmol) with drop wise at 0° C. Reaction mixture was stirred at RT for 3 h. The progress of the reaction was monitored by TLC. After consumption of starting material, the reaction mixture was evaporated under reduced pressure to obtain crude compound, the resulting crude triturated with diethyl ether to afford pure methyl N5,N5-dimethyl-L-glutaminate (3).

### Methyl N2-((S)-2-((((3-chlorobenzyl) oxy) carbonyl) amino)-3-cyclohexylpropanoyl)-N5, N5-dimethyl-L-glutaminate (5)

To a stirred solution of (S)-2-((((3-chlorobenzyl)oxy)carbonyl)amino)-3-cyclohexylpropanoic acid (4) (300 mg, 0.88 mmol) (synthesis reported in Compd-17 final report) in DMF (10 mL) was added HOBt (143 mg, 1.06 mmol), EDC.HCl (202 mg, 1.06 mmol) and DIPEA (0.48 mL, 2.65 mmol) followed by methyl N5,N5-dimethyl-L-glutaminate (3) (167 mg, 0.88 mmol) at 0° C. Reaction mixture was stirred at room temperature for 16 h. The progress of the reaction was monitored by TLC. After consumption of starting material, the reaction mixture was quenched with water (10 mL) and extracted with EtOAc (2×100 mL). Combined organic layer was washed with brine solution, dried over Na₂SO₄ and concentrated to get crude; Crude compound was purified by normal phase chromatography to afford pure compound methyl N2-((S)-2-(((3-chlorobenzyl) oxy) carbonyl) amino)-3-cyclohexylpropanoyl)-N5, N5-dimethyl-L-glutaminate (4).

### 3-chlorobenzyl ((S)-3-cyclohexyl-1-(((S)-5-(dimethylamino)-1-hydroxy-5-oxopentan-2-yl)amino)-1-oxopropan-2-yl)carbamate (6)

To a stirred solution of methyl N2-((S)-2-((((3-chlorobenzyl)oxy)carbonyl) amino)-3-cyclohexylpropanoyl)-N5,N5-dimethyl-L-glutaminate (5) (220 mg, 0.43 mmol) in DCM (10 mL) was added 2M LiBH₄ in THE (0.64 mL, 1.29 mmol) at 0° C., reaction mixture stirred for 1 h at 0° C. Reaction mixture was quenched with water (20 mL) and extracted with DCM (2×20 mL). Organic layer was washed with brine solution, dried over Na₂SO₄ and concentrated to get crude. Crude compound was purified by normal phase chromatography to afford pure 3-chlorobenzyl ((S)-3-cyclohexyl-1-(((S)-5-(dimethylamino)-1-hydroxy-5-oxopentan-2-yl) amino)-1-oxopropan-2-yl) carbamate (6).

### 3-chlorobenzyl ((S)-3-cyclohexyl-1-(((S)-5-(dimethylamino)-1,5-dioxopentan-2-yl)amino)-1-oxopropan-2-yl)carbamate A6

To a stirred solution of 3-chlorobenzyl ((S)-3-cyclohexyl-1-(((S)-5-(dimethylamino)-1-hydroxy-5-oxopentan-2-yl) amino)-1-oxopropan-2-yl) carbamate (6) (50 mg, 0.10 mmol) was dissolved in DCM (5 mL) was added Dess-Martin periodinane (88 mg, 0.19 mmol) at 0° C. and stirred at RT for 3 h. Reaction mixture was diluted with DCM (15 mL) and washed with sat. hypo solution (3×20 mL), sat. NaHCO₃ solution (3×20 mL). Organic layer was dried over anhydrous Na₂SO₄, filtered and concentrated to get crude compound. It was purified by prep HPLC to afford pure 3-chlorobenzyl ((S)-3-cyclohexyl-1-(((S)-5-(dimethylamino)-1,5-dioxopentan-2-yl)amino)-1-oxopropan-2-yl)carbamate A6.

TLC system: 10% Methanol in DCM; Rf: 0.4

LCMS (ESI): m/z 480.28 (M+H)⁺

### Synthesis of Compounds A7, A8, and A9

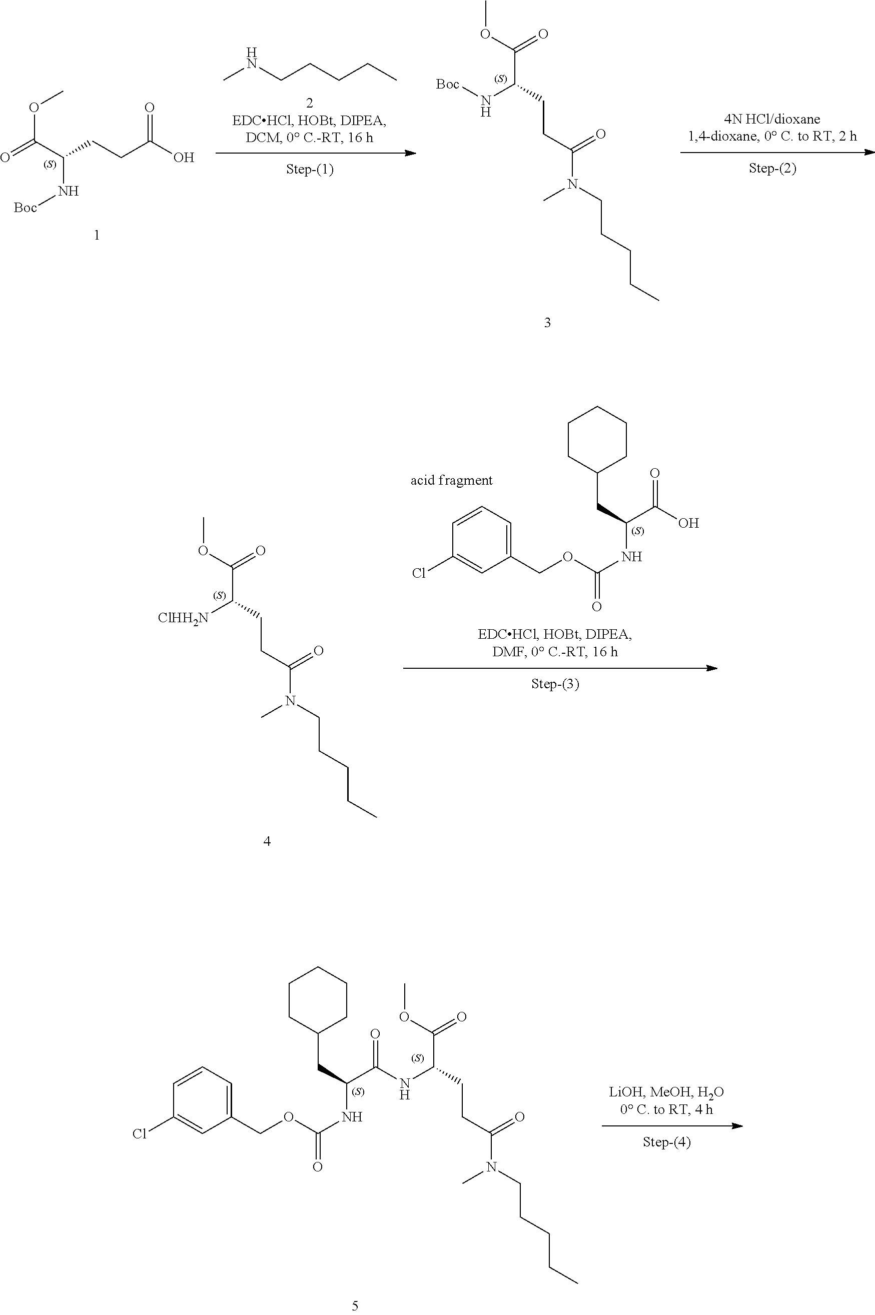

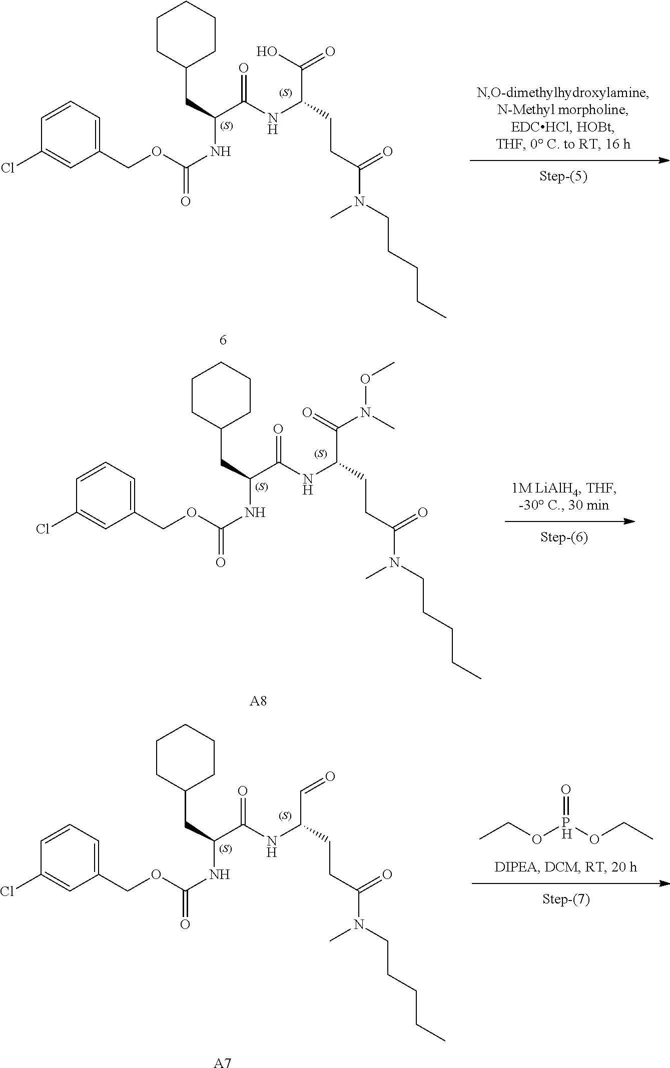

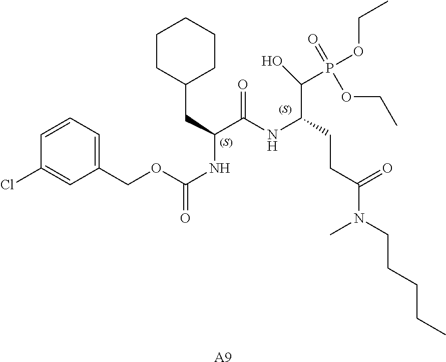

### Methyl N2-(tert-butoxycarbonyl)-N5-methyl-N5-pentyl-L-glutaminate (3)

To a stirred solution of (S)-4-((tert-butoxycarbonyl)amino)-5-methoxy-5-oxopentanoic acid (1) (1 g, 3.83 mmol) in DMF (10 mL) at 0° C. was added EDC.HCl (1.1 g, 5.74 mmol), HOBT (775 mg, 5.74 mmol) and DIPEA (2.1 mL, 11.49 mmol) and stirred 10 min, then added N-methylpentan-1-amine (2) (425 mg, 4.21 mmol) in 5 mL DMF at 0° C. and stirred at room temperature for 16 h. Reaction mixture was diluted with ice water (25 mL), extracted with EtOAc (2×30 mL). Organic layer was washed with water (20 mL), brine (20 mL) dried over anhydrous Na₂SO₄, filtered and evaporated under reduced pressure to afford crude product, this crude was purified by silica (100-200 mesh) column chromatography by eluting with pet ether to afford methyl N²-(tert-butoxycarbonyl)-N⁵-methyl-N⁵-pentyl-L-glutaminate (3).

### Methyl N⁵-methyl-N⁵-pentyl-L-glutaminate hydrochloride (4)

To a stirred solution of methyl N²-(tert-butoxycarbonyl)-N⁵-methyl-N⁵-pentyl-L-glutaminate (3) (1 g, 2.9069 mmol) in 1,4-dioxane (10 mL), was added 4N HCl in 1,4-dioxane (10 mL) at 0° C. then reaction mixture was stirred at room temperature for 3 h. After consumption of starting material by TLC, distilled off the solvent to afford crude methyl N⁵-methyl-N⁵-pentyl-L-glutaminate hydrochloride (4).

### Methyl N2-((S)-2-((((3-chlorobenzyl)oxy)carbonyl)amino)-3-cyclohexylpropanoyl)-N⁵-methyl-N⁵-pentyl-L-glutaminate (5)

To a stirred solution of (S)-2-((((3-chlorobenzyl)oxy)carbonyl)amino)-3-cyclohexylpropanoic acid (968 mg, 2.85 mmol)) (synthesis reported in Compd-17 final report) in DMF (10 mL) was added HOBt (578 mg, 4.2857 m mmol), EDC.HCl (818 mg, 4.2857 mmol) and DIPEA (1.6 mL, 8.5714 mmol) followed by methyl N⁵-methyl-N⁵-pentyl-L-glutaminate hydrochloride (4) (800 mg, 2.8571 mmol) at 0° C. Reaction mixture was stirred at room temperature for 16 h. The progress of the reaction was monitored by TLC. After consumption of starting material, the reaction mixture was quenched with water (20 mL) and extracted with EtOAc (2×30 mL). Combined organic layer was washed with brine solution, dried over Na₂SO₄ and concentrated to get crude; Crude compound was purified by normal phase chromatography to afford pure compound methyl N2-((S)-2-((((3-chlorobenzyl)oxy)carbonyl)amino)-3-cyclohexylpropanoyl)-N5-methyl-N5-pentyl-L-glutaminate (5).

### N²—((S)-2-((((3-chlorobenzyl)oxy)carbonyl)amino)-3-cyclohexylpropanoyl)-N⁵-methyl-N⁵-pentyl-L-glutamine (6)

To a stirred solution of methyl N²—((S)-2-((((3-chlorobenzyl)oxy)carbonyl)amino)-3-cyclohexylpropanoyl)-N⁵-methyl-N⁵-pentyl-L-glutaminate (5) (1 g, 1.7699 mmol) in THE (20 mL), water (10 mL) was added LiOH (127 mg, 5.3097 mmol) at 0° C. and the reaction mixture was stirred at RT for 4 h. After consumption of starting material, the reaction mixture concentrated under reduced pressure to afford crude product; this crude was acidified with 1N HCl and extracted with DCM (2×50 mL). Organic layer was washed with brine solution, dried over Na₂SO₄ and concentrated to afford N²—((S)-2-((((3-chlorobenzyl)oxy)carbonyl)amino)-3-cyclohexylpropanoyl)-N-methyl-N⁵-pentyl-L-glutamine (6).

### 3-chlorobenzyl ((S)-3-cyclohexyl-1-(((S)-1-(methoxy (methyl) amino)-5-(methyl(pentyl)amino)-1,5-dioxopentan-2-yl)amino)-1-oxopropan-2-yl)carbamate A8

To a stirred solution of N²—((S)-2-((((3-chlorobenzyl)oxy)carbonyl)amino)-3-cyclohexylpropanoyl)-N-methyl-N⁵-pentyl-L-glutamine (6) (800 mg, 1.45 mmol) in THE (10 mL) and DCM (10 mL) at 0° C. sequentially added EDC.HCl (415 mg, 2.17 mmol), HOBT (294 mg, 2.17 mmol), N-methyl morpholine (0.6 mL, 4.35 mmol) and N,O-dimethylhydroxylamine hydro chloride (170 mg, 1.74 mmol) and stirred at room temperature for 16 h. Reaction mixture was diluted with ice water (20 mL), extracted with EtOAc (2×25 mL), dried over anhydrous Na₂SO₄ and evaporated under reduced pressure. The crude residue was purified by normal phase chromatography to afford pure compound 3-chlorobenzyl ((S)-3-cyclohexyl-1-(((S)-1-(methoxy(methyl)amino)-5-(methyl(pentyl)amino)-1,5-dioxopentan-2-yl)amino)-1-oxopropan-2-yl)carbamate A8.

TLC system: 80% EtOAc in Hexane; Rf: 0.4

LCMS (ESI): m/z=595.41 (M+H)⁺

### 3-chlorobenzyl ((S)-3-cyclohexyl-1-(((S)-5-(methyl(pentyl)amino)-1,5-dioxopentan-2-yl)amino)-1-oxopropan-2-yl)carbamate A7

To a stirred solution of 3-chlorobenzyl ((S)-3-cyclohexyl-1-(((S)-1-(methoxy(methyl)amino)-5-(methyl(pentyl)amino)-1,5-dioxopentan-2-yl)amino)-1-oxopropan-2-yl)carbamate A8 (100 mg, 0.16 mmol) in THE (5 mL) at −30° C. was added 2M LiAlH₄ (0.12 mL, 0.25 mmol) and reaction mixture was stirred at −30° C. for 30 min. After consumption of starting material, diluted with ice water (50 mL) extracted with EtOAc (2×25 mL), combined organic layer was washed with water (2×20 mL), and brine (20 mL). Organic layer was dried over anhydrous Na₂SO₄ and concentrated to afford crude product; it was purified by prep HPLC to afforded 3-chlorobenzyl ((S)-3-cyclohexyl-1-(((S)-5-(methyl(pentyl)amino)-1,5-dioxopentan-2-yl)amino)-1-oxopropan-2-yl)carbamate A7.

TLC system: 50% EtOAc in Hexane; Rf: 0.3

LCMS (ESI): m/z 536.33 [M+H]⁺

### 3-chlorobenzyl ((2S)-3-cyclohexyl-1-(((2S)-1-(diethoxyphosphoryl)-1-hydroxy-5-(methyl(pentyl)amino)-5-oxopentan-2-yl)amino)-1-oxopropan-2-yl)carbamate A9

To a stirred solution of 3-chlorobenzyl ((S)-3-cyclohexyl-1-(((S)-5-(methyl(pentyl)amino)-1,5-dioxopentan-2-yl)amino)-1-oxopropan-2-yl)carbamate A7 (130 mg, 0.2429 mmol) in DCM (5 mL) added DIPEA (0.13 mL, 0.72 mmol) followed by added diethyl phosphite (0.1 mL, 0.72 mmol). Then reaction mixture stirred at RT for 16 h and then reaction mixture quenched with ammonium chloride (15 mL) and extracted with DCM (2×20 mL). Combined organic layer dried over anhydrous Na₂SO₄, and evaporated to afford crude residue. It was purified prep HPLC to afforded 3-chlorobenzyl ((2S)-3-cyclohexyl-1-(((2S)-1-(diethoxyphosphoryl)-1-hydroxy-5-(methyl (pentyl) amino)-5-oxopentan-2-yl) amino)-1-oxopropan-2-yl) carbamate A9.

TLC system: 80% EtOAc in Hexane; Rf: 0.5

LCMS (ESI): m/z 674.3 (M+H)⁺

### Synthesis of Compounds A10 and A12

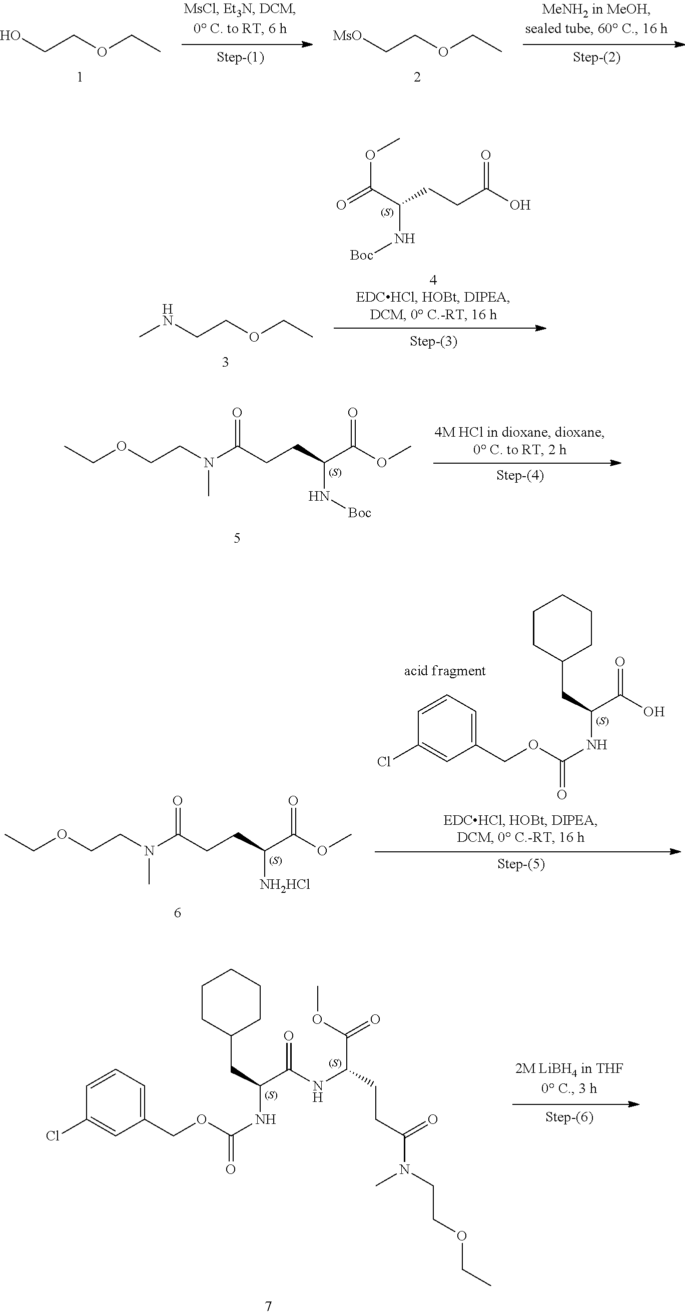

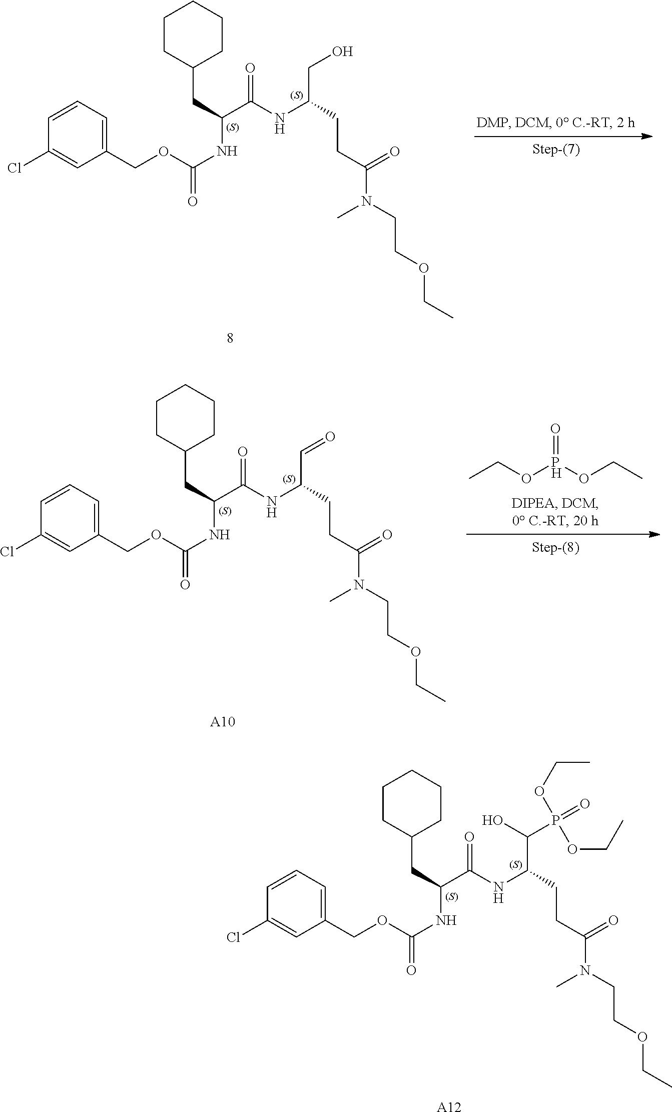

### 2-ethoxyethyl methanesulfonate (2)

To a stirred solution of 2-ethoxyethan-1-ol (1) (1 g, 11.09 mmol) in DCM (50 mL) at 0° C. was added NEt₃ (3.2 mL, 22.19 mmol), mesyl chloride (1 mL, 13.31 mmol) and stirred at RT for 6 h. After consumption of starting material, reaction mixture was quenched with water (15 mL) and extracted with DCM (2×15 mL). Combined organic layer was washed with water (15 mL), brine (15 mL) dried over anhydrous Na₂SO₄, filtered and evaporated under reduced pressure to afford crude 2-ethoxyethyl methanesulfonate (2).

### 2-ethoxy-N-methylethan-1-amine (3)

To a stirred solution of 2-ethoxyethyl methanesulfonate (2) (2 g, 11.90 mmol) in methylamine in methanol (20 mL) was stirred at 60° C. for 16 h. After consumption of starting material, reaction mixture evaporated under reduced pressure to afford crude 2-ethoxy-N-methylethan-1-amine (3). Methyl N²-(tert-butoxycarbonyl)-N⁵-(2-ethoxyethyl)-N⁵-methyl-D-glutaminate (5)

To a stirred solution of (S)-4-((tert-butoxycarbonyl)amino)-5-methoxy-5-oxopentanoic acid (4) (2.28 g, 8.73 mmol) in DMF (10 mL) at 0° C. was added EDC.HCl (2.78 g, 14.56 mmol), HOBT (1.96 g, 14.56 mmol), DIPEA (5.6 mL, 29.12 mmol) and 2-ethoxy-N-methylethan-1-amine (3) (1 g, 9.71 mmol) sequentially and stirred at room temperature for 16 h. Reaction mixture was diluted with ice water (20 mL) extracted with EtOAc (2×25 mL) dried over anhydrous Na₂SO₄ and evaporated under reduced pressure. The crude residue was purified by grace normal phase by using 40% EtOAc in pet ether as eluent to afford Methyl N²-(tert-butoxycarbonyl)-N⁵-(2-ethoxyethyl)-N⁵-methyl-D-glutaminate (5)

### Methyl N⁵-(2-ethoxyethyl)-N⁵-methyl-D-glutaminate hydrochloride (6)

To a stirred solution of Methyl-N²-(tert-butoxycarbonyl)-N5-(2-ethoxyethyl)-N⁵-methyl-D-glutaminate (5) (1.8 g, 5.20 mmol) in 1,4-dioxane (20 mL) at 0° C. was added 4 M HCl in dioxane (10 mL) by drop wise. Reaction mixture was stirred at RT for 2 h. The progress of the reaction was monitored by TLC. After consumption of starting material, the reaction mixture was evaporated under reduced pressure to obtain crude compound, the resulting crude triturated with diethyl ether to afford methyl N⁵-(2-ethoxyethyl)-N⁵-methyl-D-glutaminate hydrochloride (6).

### Methyl N²—((S)-2-((((3-chlorobenzyl)oxy)carbonyl)amino)-3-cyclohexylpropanoyl)-N⁵-(2-ethoxyethyl)-N⁵-methyl-L-glutaminate (7)

To a stirred solution of (S)-2-((((3-chlorobenzyl)oxy)carbonyl)amino)-3-cyclohexylpropanoic acid (2.2 g, 6.50 mmol) in DMF (20 mL) was added EDC-HCl (1.86 g, 9.75 mmol), HOBT (1.31 g, 9.75 mmol), DIPEA (3.6 mL, 19.51 mmol) and methyl N⁵-(2-ethoxyethyl)-N⁵-methyl-D-glutaminate hydrochloride (1.6 g, 6.50 mmol) at 0° C. sequentially and stirred at room temperature for 16 h. Reaction mixture was diluted with ice water (40 mL), extracted with EtOAc (2×40 mL), dried over anhydrous sodium sulfate and evaporated under reduced pressure. The crude residue was purified by grace RP, compound eluted at 60% MeCN in 0.1% formic acid in water to afford methyl N²—((S)-2-((((3-chlorobenzyl)oxy)carbonyl)amino)-3-cyclohexylpropanoyl)-N⁵-(2-ethoxyethyl)-N⁵-methyl-L-glutaminate (7).

### 3-chlorobenzyl ((S)-3-cyclohexyl-1-(((S)-5-((2-ethoxyethyl)(methyl)amino)-1-hydroxy-5-oxopentan-2-yl)amino)-1-oxopropan-2-yl)carbamate (8)

To a stirred solution of Methyl-N²—((S)-2-((((3-chlorobenzyl)oxy)carbonyl)amino)-3-cyclohexylpropanoyl)-N⁵-(2-ethoxyethyl)-N⁵-methyl-L-glutaminate (7) (1.5 g, 2.64 mmol) in DCM (30 mL) at 0° C. was added 2M LiBH₄ in THE (3.96 mL, 7.93 mmol) then reaction mixture was stirred for 3 h at 0° C. Reaction mixture was quenched with water (20 mL) and extracted with DCM (2×20 mL). Combined organic layer was washed with brine solution, dried over Na₂SO₄ and concentrated to get crude residue. It was purified by normal phase chromatography to afford pure 3-chlorobenzyl ((S)-3-cyclohexyl-1-(((S)-5-((2-ethoxyethyl)(methyl)amino)-1-hydroxy-5-oxopentan-2-yl)amino)-1-oxopropan-2-yl)carbamate (8)

### 3-chlorobenzyl ((S)-3-cyclohexyl-1-(((S)-5-((2-ethoxyethyl)(methyl)amino)-1,5-dioxopentan-2-yl)amino)-1-oxopropan-2-yl)carbamate A10

To a stirred solution of 3-chlorobenzyl ((S)-3-cyclohexyl-1-(((S)-5-((2-ethoxyethyl)(methyl)amino)-1-hydroxy-5-oxopentan-2-yl)amino)-1-oxopropan-2-yl)carbamate (8) (200 mg, 0.370 mmol) was dissolved in DCM (2 mL) at 0° C. and added Dess-Martin periodinane (314 mg, 0.74 mmol) and stirred at RT for 2 h. Reaction mixture was diluted with DCM (50 mL) and washed with sat. hypo solution (3×20 mL), sat. NaHCO₃ solution (3×20 mL). Organic layer was dried over anhydrous Na₂SO₄, filtered and concentrated to get crude residue. It was purified by prep HPLC to afford pure 3-chlorobenzyl ((S)-3-cyclohexyl-1-(((S)-5-((2-ethoxyethyl)(methyl)amino)-1,5-dioxopentan-2-yl)amino)-1-oxopropan-2-yl)carbamate A10.

TLC system: 10% MeOH in DCM; Rf: 0.4

LCMS (ESI): m/z 538.35. (M+H)⁺

### 3-chlorobenzyl ((2S)-3-cyclohexyl-1-(((2S)-1-(diethoxyphosphoryl)-5-((2-ethoxyethyl)(methyl)amino)-1-hydroxy-5-oxopentan-2-yl)amino)-1-oxopropan-2-yl)carbamate A12

To a stirred solution of 3-chlorobenzyl ((S)-3-cyclohexyl-1-(((S)-5-((2-ethoxyethyl)(methyl)amino)-1,5-dioxopentan-2-yl)amino)-1-oxopropan-2-yl)carbamate (200 mg, 0.37 mmol) in DCM (2 mL) at 0° C. was added DIPEA (0.19 mL, 1.117 mmol) followed by diethyl phosphite (0.2 mL, 1.117 mmol) then reaction mixture stirred at RT for 16 h. RM was quenched with water (15 mL) and extracted with DCM (2×20 mL). Combined organic layer dried over anhydrous Na₂SO₄, evaporated to afford crude residue. It was purified prep HPLC to afforded 3-chlorobenzyl ((2S)-3-cyclohexyl-1-(((2S)-1-(diethoxyphosphoryl)-5-((2-ethoxyethyl)(methyl)amino)-1-hydroxy-5-oxopentan-2-yl)amino)-1-oxopropan-2-yl)carbamate A12.

TLC system: 5% MeOH in DCM; Rf: 0.4

LCMS (ESI): m/z 676.43 (M+H)⁺

### Synthesis of Compounds A25 and A11

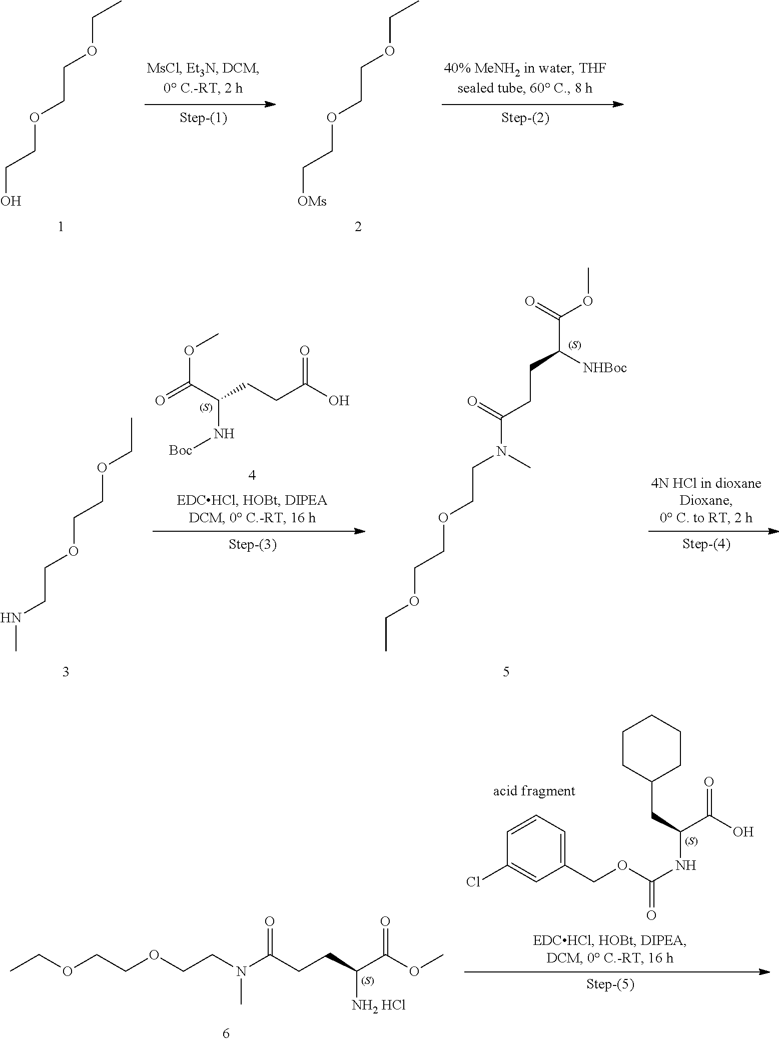

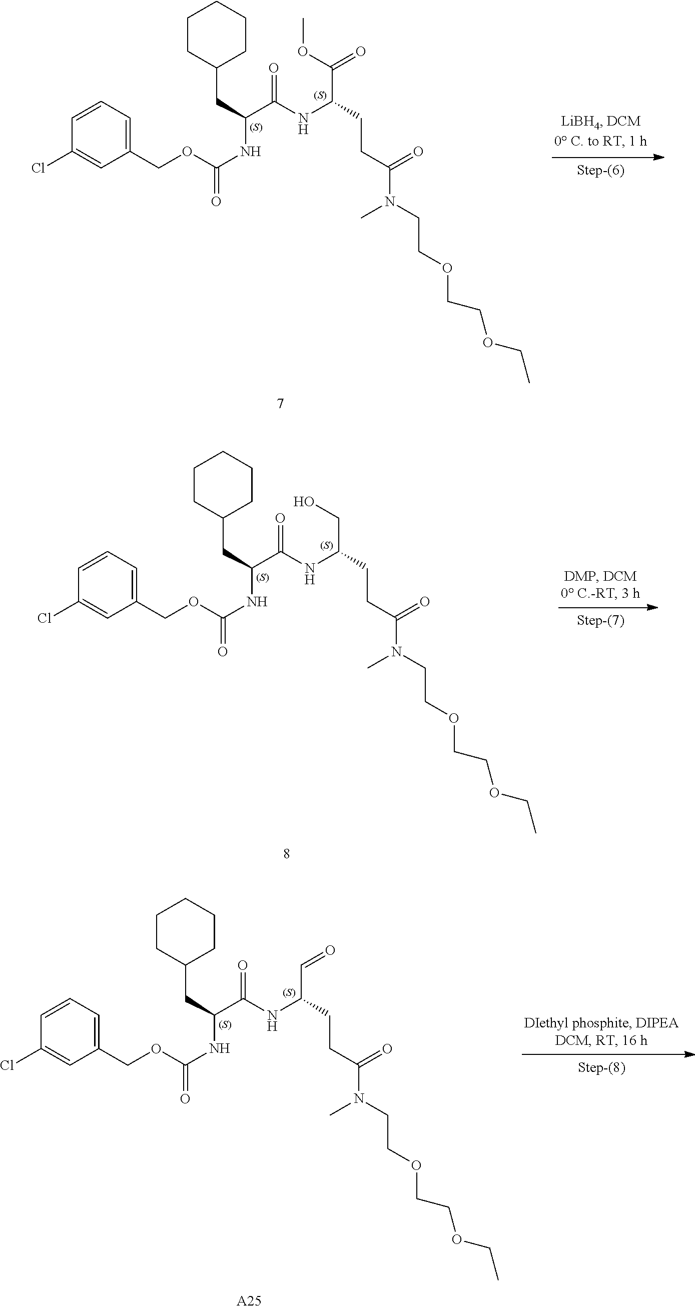

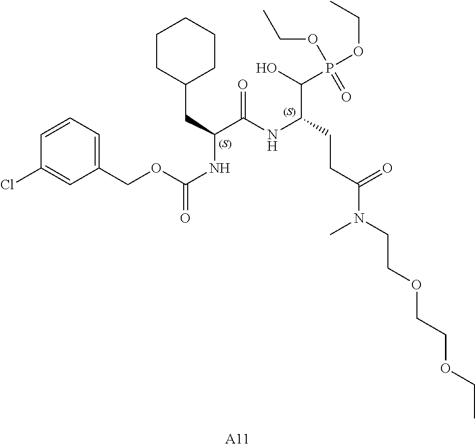

### 2-(2-Ethoxyethoxy)ethyl methanesulfonate (2)

To a stirred solution of 2-(2-ethoxyethoxy)ethan-1-ol (1) (1 g, 7.462 mmol) in DCM (20 mL) at 0° C. was added Et₃N (2.1 mL, 14.92 mmol), methanesulfonyl chloride (689 μL, 8.95 mmol) in portion wise over 5 minutes under nitrogen atmosphere and the reaction mixture was stirred at RT for 2 h. After 2 h, the reaction mixture was diluted with DCM (30 mL) and washed with water (10 mL) and brine solution (10 mL). Organic layer was dried over anhydrous Na₂SO₄ and concentrated under vacuo to afford 2-(2-ethoxyethoxy)ethyl methanesulfonate (2). The compound was taken for next step without any purification.

### 2-(2-Ethoxyethoxy)-N-methylethan-1-amine (3)

To a stirred solution of 2-(2-ethoxyethoxy)ethyl methanesulfonate (2) (5 g, 23.58 mmol) in THE (2 mL) was added 40% methyl amine in H₂O (25 mL) and heated to 60° C. for 8 h. Then the mixture was evaporated to dryness to afford 2-(2-ethoxyethoxy)-N-methylethan-1-amine (3). The compound was taken for next step without any purification.

### Methyl N²-(tert-butoxycarbonyl)-N⁵-(2-(2-ethoxyethoxy)ethyl)-N⁵-methyl-L-glutaminate (5)

To a stirred solution of (S)-4-((tert-butoxycarbonyl)amino)-5-methoxy-5-oxopentanoic acid (4) (4 g, 15.32 mmol) in DMF (40 mL) at 0° C. was added HOBt (3.1 g, 22.98 mmol), EDC.HCl (4.39 g, 22.98 mmol) and DIPEA (8.16 mL, 45.97 mmol) followed by 2-(2-ethoxyethoxy)-N-methylethan-1-amine (3) (4.5 g, 30.65 mmol) and the reaction mixture was stirred at room temperature for 16 h. The progress of the reaction was monitored by TLC. After 16 h, the reaction mixture was quenched with ice water (50 mL) and extracted with EtOAc (2×200 mL). Combined organic layer was washed with brine solution (50 mL), dried over Na₂SO₄ and concentrated to get crude residue. The crude was purified by normal phase silica chromatography (100-200 mesh) to afford methyl N²-(tert-butoxycarbonyl)-N⁵-(2-(2-ethoxyethoxy)ethyl)-N⁵-methyl-L-glutaminate (5).

### Methyl N⁵-(2-(2-ethoxyethoxy)ethyl)-N⁵-methyl-L-glutaminate hydrochloride (6)

To a stirred solution of methyl N2-(tert-butoxycarbonyl)-N⁵-(2-(2-ethoxyethoxy)ethyl)-N⁵-methyl-L-glutaminate (5) (1 g, 2.564 mmol) in 1,4-dioxane (10 mL) at 0° C. was added 4N HCl in dioxane (10 mL) drop wise and the resulting reaction mixture was stirred for 2 h at RT. After consumption of starting material, the volatiles were removed under vacuo. Crude residue was triturated with diethylether twice (2×20 mL) to afford methyl N⁵-(2-(2-ethoxyethoxy)ethyl)-N⁵-methyl-L-glutaminate hydrochloride (6).

### Methyl N²—((S)-2-((((3-chlorobenzyl)oxy)carbonyl)amino)-3-cyclohexylpropanoyl)-N⁵-(2-(2-ethoxyethoxy)ethyl)-N5-methyl-L-glutaminate (7)

To a stirred solution of (S)-2-((((3-chlorobenzyl)oxy)carbonyl)amino)-3-cyclohexylpropanoic acid (acid fragment) (750 mg, 2.212 mmol) in DMF (7.5 mL) at 0° C. was added HOBt (448 mg, 3.318 mmol), EDC.HCl (633 mg, 3.318 mmol) and DIPEA (1.16 mL, 6.636 mmol) followed by methyl N⁵-(2-(2-ethoxyethoxy)ethyl)-N⁵-methyl-L-glutaminate hydrochloride (6, 865 mg, 2.654 mmol) and the reaction mixture was stirred at room temperature for 16 h. The progress of the reaction was monitored by TLC and LCMS. After 16 h, the reaction mixture was quenched with ice water (20 mL) and extracted with EtOAc (2×50 mL). Combined organic layer was washed with brine solution (30 mL), the organic layer was dried over Na₂SO₄ and concentrated to get crude. The crude compound was purified by normal phase silica chromatography to afford pure compound methyl N²-(tert-butoxycarbonyl)-N⁵-(2-(2-ethoxyethoxy)ethyl)-N⁵-methyl-L-glutaminate (7).

### 3-Chlorobenzyl ((13S,16S)-17-cyclohexyl-13-(hydroxymethyl)-9-methyl-10,15-dioxo-3,6-dioxa-9,14-diazaheptadecan-16-yl)carbamate (8)

To a stirred solution of methyl N²—((S)-2-((((3-chlorobenzyl)oxy)carbonyl)amino)-3-cyclohexylpropanoyl)-N⁵-(2-(2-ethoxyethoxy)ethyl)-N⁵-methyl-L-glutaminate (7) (500 mg, 0.8183 mmol) in DCM (5 mL) at 0° C. was added 2M LiBH₄ in THE (0.614 mL, 1.22 mmol) then reaction mixture was stirred at same temperature for 1 h. The progress of the reaction was monitored by TLC and LCMS. After 1 h, the reaction mixture was quenched with ice water (20 mL) and extracted with DCM (2×20 mL). The combined organic layer was washed with brine solution (20 mL), dried over Na₂SO₄ and concentrated to get crude residue. The crude was purified by normal phase silica chromatography (100-200 mesh) to afford pure 3-Chlorobenzyl ((13S,16S)-17-cyclohexyl-13-(hydroxymethyl)-9-methyl-10,15-dioxo-3,6-dioxa-9,14-diazaheptadecan-16-yl)carbamate (8).

### 3-Chlorobenzyl ((13S,16S)-17-cyclohexyl-13-formyl-9-methyl-10,15-dioxo-3,6-dioxa-9,14-diazaheptadecan-16-yl)carbamate A25

To a stirred solution of 3-Chlorobenzyl ((13S,16S)-17-cyclohexyl-13-(hydroxymethyl)-9-methyl-10,15-dioxo-3,6-dioxa-9,14-diazaheptadecan-16-yl)carbamate (8) (350 mg, 0.6 mmol) was dissolved in DCM (7 mL) at 0° C. was added Dess-Martin periodinane (763 mg, 1.8 mmol) and stirred at RT for 3 h. The progress of the reaction was monitored by TLC and LCMS. Reaction mixture was diluted by DCM (15 mL) and washed with sat. Hypo solution (3×20 mL), sat. NaHCO₃ solution (3×20 mL). Organic layer was dried over anhydrous Na₂SO₄, filtered and concentrated to get crude compound. It was purified by prep HPLC to afford pure 3-Chlorobenzyl ((13S,16S)-17-cyclohexyl-13-formyl-9-methyl-10,15-dioxo-3,6-dioxa-9,14-diazaheptadecan-16-yl)carbamate A25.

TLC system: 10% Methanol in DCM; Rf: 0.5

LCMS (ESI): m/z 582.45 (M+H)⁺

### 3-Chlorobenzyl ((13S,16S)-17-cyclohexyl-13-((diethoxyphosphoryl)(hydroxy)methyl)-9-methyl-10,15-dioxo-3,6-dioxa-9,14-diazaheptadecan-16-yl)carbamate A11

To a stirred solution of 3-chlorobenzyl ((13S,16S)-17-cyclohexyl-13-formyl-9-methyl-10,15-dioxo-3,6-dioxa-9,14-diazaheptadecan-16-yl)carbamate A25 (150 mg, 0.25 mmol) in DCM (3 mL) was added DIPEA (0.14 mL, 0.77 mmol) and diethyl phosphite (0.105 mL, 0.77 mmol). Then reaction mixture was stirred at RT for 16 h. The progress of the reaction was monitored by TLC and LCMS. The reaction mixture was diluted by DCM (50 mL) and washed with water (2×50 mL) and brine solution (20 mL). Organic layer was dried over anhydrous Na₂SO₄, and evaporated to afford crude residue. It was purified by prep HPLC and afforded 3-chlorobenzyl ((13S,16S)-17-cyclohexyl-13 ((diethoxyphosphoryl)(hydroxy)methyl)-9-methyl-10,15-dioxo-3,6-dioxa-9,14-diazaheptadecan-16-yl)carbamate A11.

TLC system: 10% MeOH in DCM; Rf: 0.45

LCMS (ESI): m/z 720.46 (M+H)⁺

### Synthesis of Compound 20

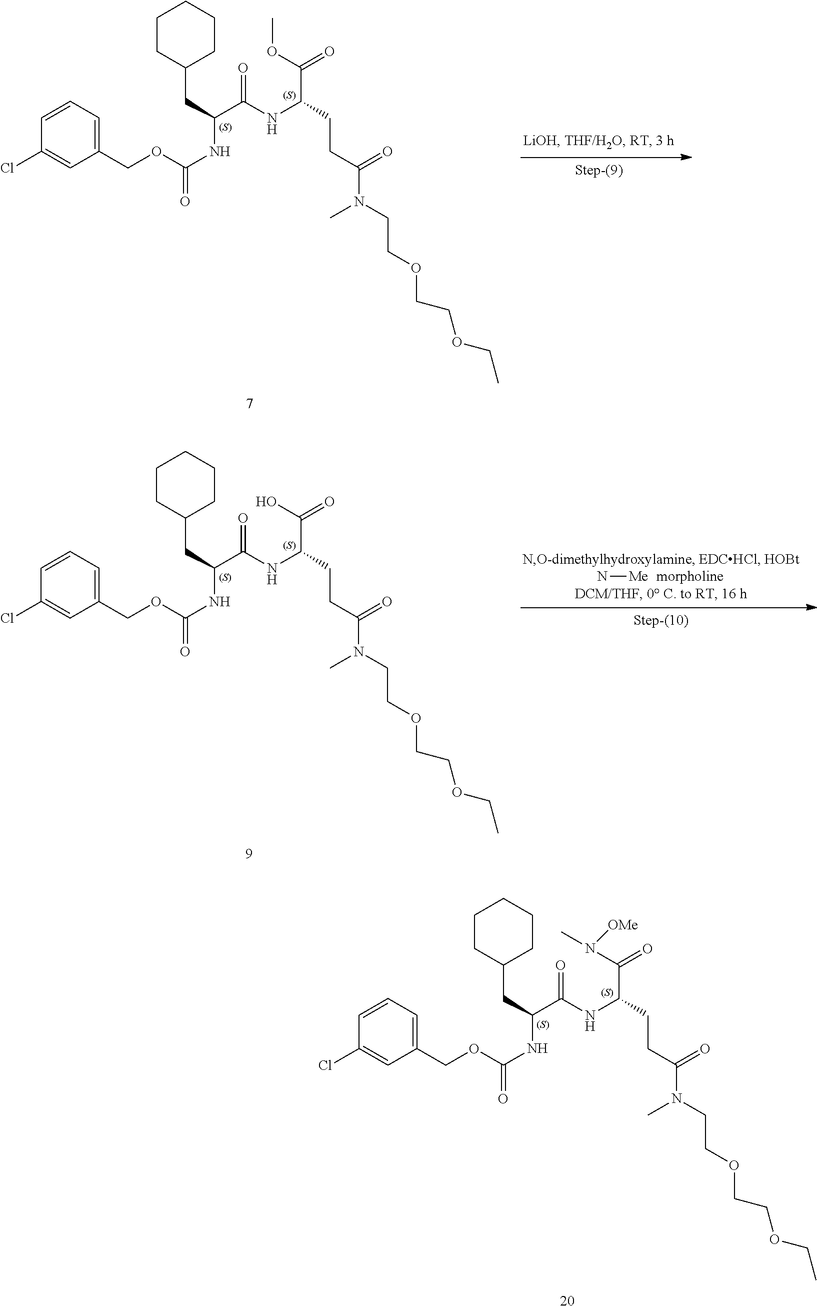

### N²—((S)-2-((((3-Chlorobenzyl)oxy)carbonyl)amino)-3-cyclohexylpropanoyl)-N⁵-(2-(2-ethoxyethoxy)ethyl)-N⁵-methyl-L-glutamine (9)

To a stirred solution of methyl N²—((S)-2-((((3-chlorobenzyl)oxy)carbonyl)amino)-3-cyclohexylpropanoyl)-N⁵-(2-(2-ethoxyethoxy)ethyl)-N⁵-methyl-L-glutaminate (7) (250 mg, 0.40916 mmol) in THE (2.5 mL) and water (2.5 mL), was added lithium hydroxide (30 mg, 1.2274 mmol) at RT and stirred at room temperature for 3 h. The progress of the reaction was monitored by TLC and LCMS. After 3 h, the reaction mixture completely distilled under reduced pressure, crude compound acidified with 1N HCl solution up to pH ˜3-4, and extracted with DCM (2×20 mL), combined organic layers were dried over sodium sulfate, and concentrated under reduced pressure to afford crude compound N²—((S)-2-((((3-chlorobenzyl)oxy)carbonyl)amino)-3-cyclohexylpropanoyl)-N⁵-(2-(2-ethoxyethoxy)ethyl)-N⁵-methyl-L-glutamine (9).

### 3-Chlorobenzyl ((13S,16S)-17-cyclohexyl-13-(methoxy(methyl)carbamoyl)-9-methyl-10,15-dioxo-3,6-dioxa-9,14-diazaheptadecan-16-yl)carbamate 20

To a stirred solution of N²—((S)-2-((((3-chlorobenzyl)oxy)carbonyl)amino)-3-cyclohexylpropanoyl)-N⁵-(2-(2-ethoxyethoxy)ethyl)-N⁵-methyl-L-glutamine (9) (250 mg, 0.41876 mmol) in THE (2.5 mL) and DCM (2.5 mL), was added EDC.HCl (95 mg, 0.5025 mmol), HOBT (67 mg, 0.50257 mmol), N-methylmorpholine (0.07 mL, 0.50257 mmol) and N,O-dimethylhydroxylamine (48 mg, 0.50257 mmol) subsequently at 0° C. The resulting solution was stirred at room temperature for 16 h. The progress of the reaction was monitored by TLC and LCMS. After 16 h, the reaction mixture was diluted with ice water (10 mL), extracted with DCM (2×20 mL). The combined organic layers were dried over sodium sulfate, filtered and evaporated under reduced pressure. The crude residue was purified by prep HPLC and afforded 3-Chlorobenzyl ((13S,16S)-17-cyclohexyl-13-(methoxy(methyl)carbamoyl)-9-methyl-10,15-dioxo-3,6-dioxa-9,14-diazaheptadecan-16-yl)carbamate 20.

TLC system: 10% Methanol in DCM; Rf: 0.6

LCMS (ESI): m/z 641.47 (M+H)⁺

### Synthesis of Compounds A18 and A13

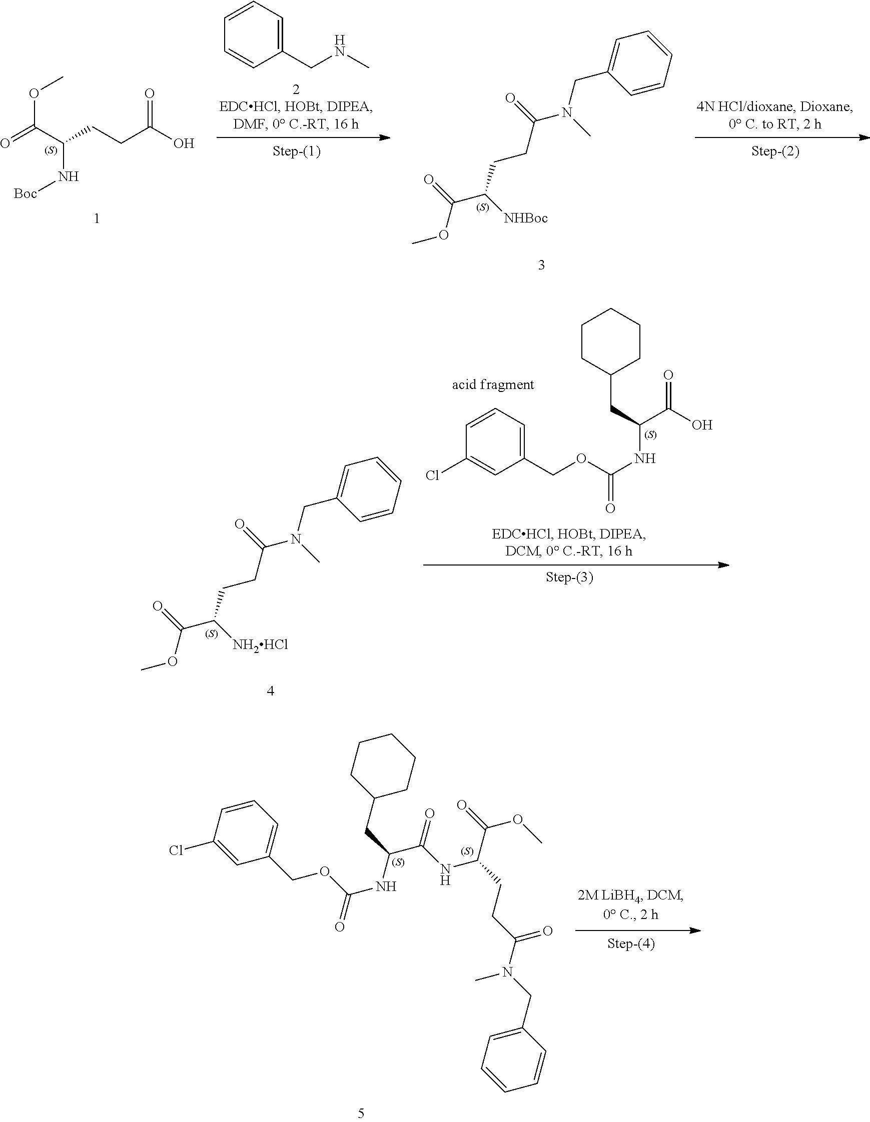

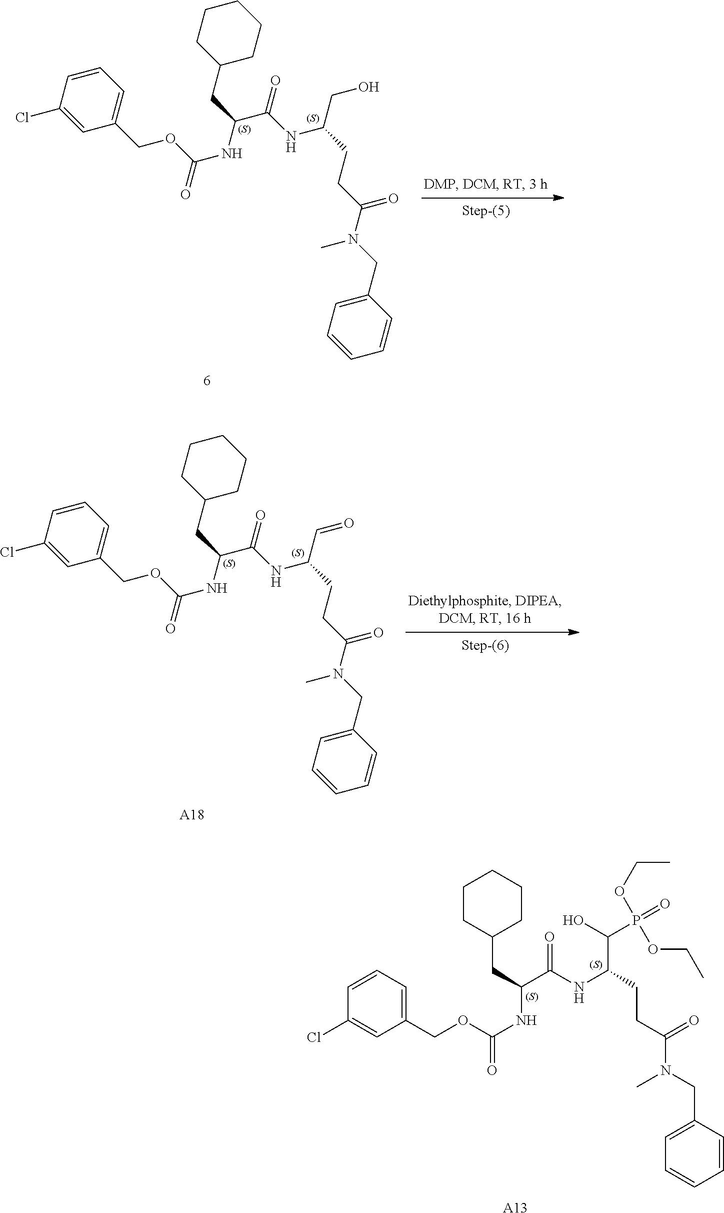

### Methyl N⁵-benzyl-N²-(tert-butoxycarbonyl)-N⁵-methyl-L-glutaminate (1)

At 0° C., to a stirred solution of (S)-4-((tert-butoxycarbonyl)amino)-5-methoxy-5-oxopentanoic acid (1) (2 g, 7.66 mmol) in DMF (20 mL) was added EDC.HCl (2.1 g, 11.49 mmol), HOBT (1.5 g, 11.49 mmol), DIPEA (4 mL, 22.98 mmol) and N-methyl-1-phenylmethanamine (2) (1.1 g, 9.19 mmol) sequentially then stirred at room temperature for 16 h. The progress of the reaction was monitored by TLC and LCMS. Reaction mixture was diluted with ice water (100 mL), extracted with EtOAc (2×55 mL), dried over anhydrous Na₂SO₄ and evaporated under reduced pressure. The crude residue was purified by silica gel column (grace normal phase) using 40% EtOAc in pet ether to afford methyl N⁵-benzyl-N²-(tert-butoxycarbonyl)-N⁵-methyl-L-glutaminate (3).

### Methyl N⁵-benzyl-N⁵-methyl-L-glutaminate hydrochloride (4)

To a stirred solution of methyl N⁵-benzyl-N²-(tert-butoxycarbonyl)-N⁵-methyl-L-glutaminate (3) (2 g, 5.49 mmol) in 1,4-dioxane (20 mL) was added 4M HCl in dioxane (10 mL) drop wise at 0° C. and stirred at RT for 2 h. The progress of the reaction was monitored by TLC. After consumption of starting material, the reaction mixture was evaporated under reduced pressure followed by trituration with diethyl ether to afford methyl N⁵-benzyl-N⁵-methyl-L-glutaminate hydrochloride (4). The product directly used for next step without further purification.

### Methyl N²—((S)-2-((((3-chlorobenzyl) oxy) carbonyl) amino)-3-cyclohexylpropanoyl)-N⁵-(2-ethoxyethyl)-N⁵-methyl-L-glutaminate (5)

To a stirred solution of (S)-2-((((3-chlorobenzyl)oxy)carbonyl)amino)-3-cyclohexylpropanoic acid (1 g, 2.95 mmol) in DMF (10 mL) at 0° C. was added EDC.HCl (847 mg, 4.437 mmol), HOBT (603 mg, 4.437 mmol), DIPEA (1.4 mL, 8.87 mmol) and methyl N⁵-benzyl-N⁵-methyl-L-glutaminate hydrochloride (1 g, 3.35 mmol) simultaneously and stirred at room temperature for 16 h. The progress of the reaction was monitored by TLC and LCMS. After 16 h, the reaction mixture was quenched with ice water (60 mL) and extracted with ethyl acetate (2×30 mL), dried over sodium sulfate and evaporated under reduced pressure. The crude residue was purified by silica gel column using grace NP by eluting with 3% methanol in DCM to afford methyl N⁵-benzyl-N²—((S)-2-((((3-chlorobenzyl) oxy) carbonyl) amino)-3-cyclohexylpropanoyl)-N⁵-methyl-L-glutaminate (5).

### 3-Chlorobenzyl ((S)-1-(((S)-5-(benzyl (methyl) amino)-1-hydroxy-5-oxopentan-2-yl) amino)-3-cyclohexyl-1-oxopropan-2-yl) carbamate (6)

To a stirred solution of methyl N⁵-benzyl-N²—((S)-2-((((3-chlorobenzyl)oxy)carbonyl)amino)-3-cyclohexylpropanoyl)-N⁵-methyl-L-glutaminate (5) (800 mg, 1.36 mmol) in DCM (15 mL) at 0° C. was added 2M LiBH₄ in THE (2 mL, 4.102 mmol) and reaction mixture stirred at RT for 2 h. The progress of the reaction was monitored by TLC and LCMS. Then the reaction mixture was quenched with water (30 mL) and extracted with DCM (2×20 mL). Combined organic layer was washed with brine solution, dried over Na₂SO₄ and concentrated to get crude residue. It was purified by silica gel column chromatography to afford 3-chlorobenzyl ((S)-1-(((S)-5-(benzyl (methyl) amino)-1-hydroxy-5-oxopentan-2-yl) amino)-3-cyclohexyl-1-oxopropan-2-yl) carbamate (6).

### 3-Chlorobenzyl ((S)-1-(((S)-5-(benzyl (methyl) amino)-1, 5-dioxopentan-2-yl) amino)-3-cyclohexyl-1-oxopropan-2-yl) carbamate A18

To a stirred solution of 3-chlorobenzyl ((S)-1-(((S)-5-(benzyl(methyl)amino)-1-hydroxy-5-oxopentan-2-yl)amino)-3-cyclohexyl-1-oxopropan-2-yl)carbamate (6) (200 mg, 0.359 mmol) in DCM (5 mL) at 0° C. was added Dess-Martin periodinane (456 mg, 1.077 mmol) and stirred at RT for 3 h. The progress of the reaction was monitored by TLC and LCMS. Reaction mixture was diluted with DCM (25 mL) and washed with sat. Hypo solution (3×20 mL) followed by saturated NaHCO₃ solution (3×20 mL). Organic layer was dried over anhydrous Na₂SO₄, filtered and concentrated to get crude residue. It was purified by prep HPLC to afford 3-chlorobenzyl ((S)-1-(((S)-5-(benzyl (methyl) amino)-1, 5-dioxopentan-2-yl) amino)-3-cyclohexyl-1-oxopropan-2-yl) carbamate A18.

TLC system: 10% MeOH in DCM; Rf: 0.4

LCMS (ESI): m/z 556.37. (M+H)⁺

### 3-Chlorobenzyl ((2S)-1-(((2S)-5-(benzyl (methyl) amino)-1-(diethoxyphosphoryl)-1-hydroxy-5-oxopentan-2-yl) amino)-3-cyclohexyl-1-oxopropan-2-yl) carbamate A13

To a stirred solution of 3-chlorobenzyl ((S)-1-(((S)-5-(benzyl(methyl)amino)-1,5-dioxopentan-2-yl)amino)-3-cyclohexyl-1-oxopropan-2-yl)carbamate (200 mg, 0.36 mmol) in DCM (5 mL) at 0° C. was added DIPEA (0.18 mL, 1.08 mmol) followed by diethylphosphite (0.2 mL, 1.08 mmol) then stirred at RT for 16 h. The progress of the reaction was monitored by TLC and LCMS. After 16 h, the reaction mixture was quenched with water (15 mL) and extracted with DCM (2×20 mL). Combined organic layer was dried over anhydrous Na₂SO₄, and evaporated to afford crude residue. It was purified prep HPLC to afforded 3-chlorobenzyl ((2S)-1-(((2S)-5-(benzyl (methyl) amino)-1-(diethoxyphosphoryl)-1-hydroxy-5-oxopentan-2-yl) amino)-3-cyclohexyl-1-oxopropan-2-yl) carbamate A13.

TLC system: 10% MeOH in DCM; Rf: 0.4

LCMS (ESI): m/z 694.2 (M+H)⁺

### Synthesis of Compounds A19 and A14

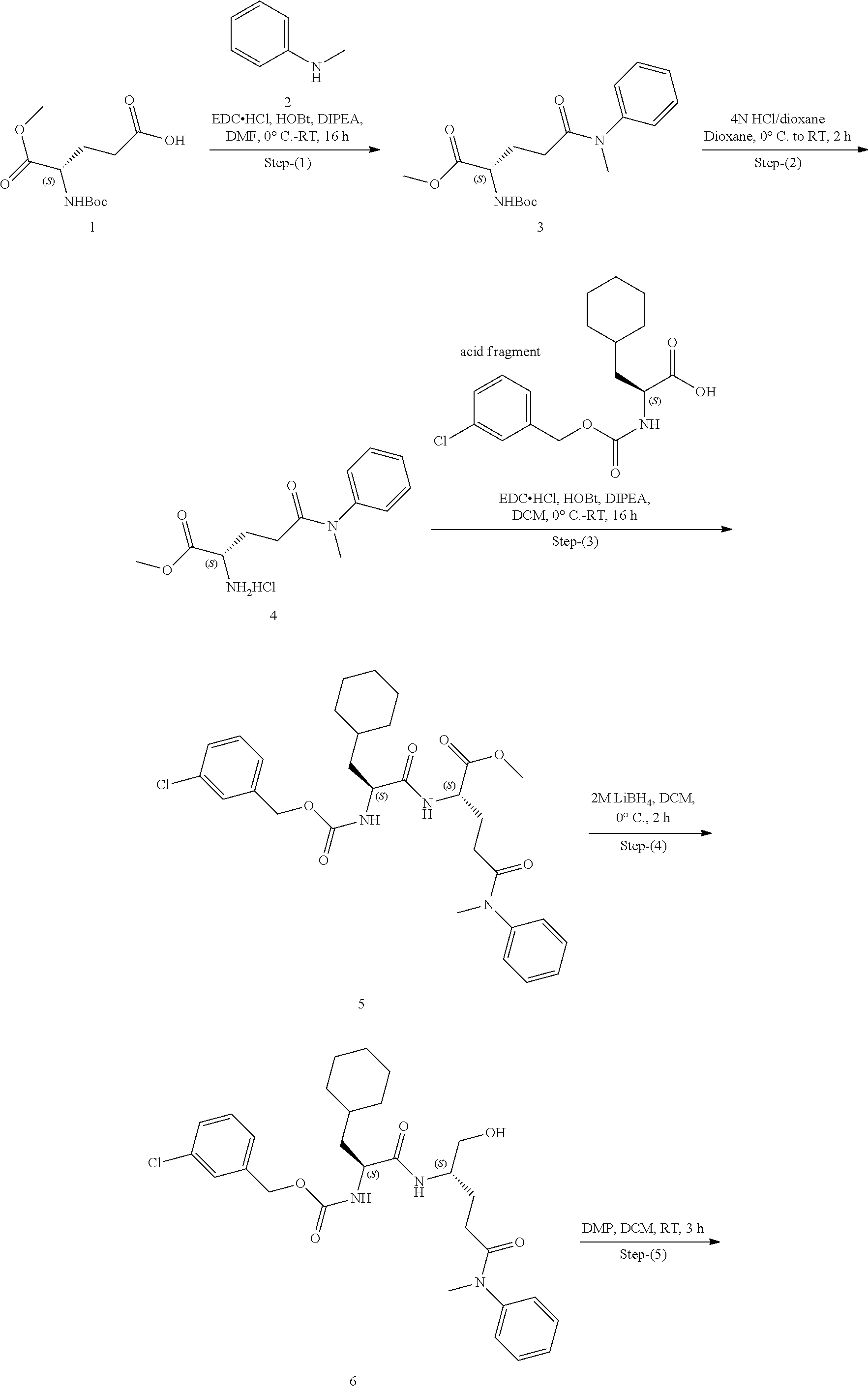

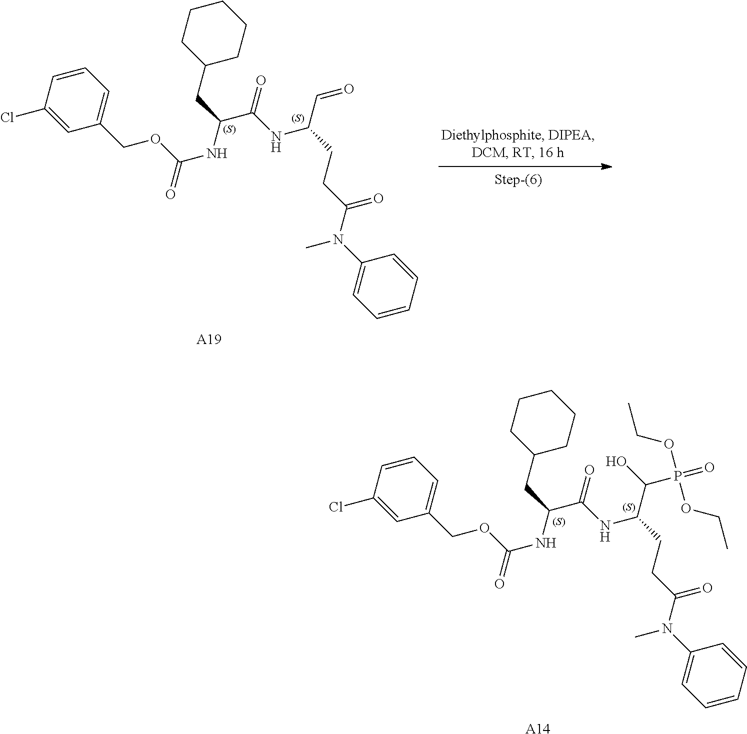

### Methyl N2-(tert-butoxycarbonyl)-N5-methyl-N5-phenyl-L-glutaminate (3)

At 0° C., to a stirred solution of (S)-4-((tert-butoxycarbonyl)amino)-5-methoxy-5-oxopentanoic acid (1) (2 g, 7.65 mmol) in DMF (20 mL) was added EDC.HCl (2.1 g, 11.494 mmol), HOBT (1.5 g, 11.494 mmol), DIPEA (4 mL, 22.98 mmol) and N-methylaniline (2) (819 mg, 7.66 mmol) and stirred at room temperature for 16 h. The progress of the reaction was monitored by TLC and LCMS. Reaction mixture was diluted with ice water (40 mL), extracted with ethyl acetate (2×40 mL), organic layer was dried over sodium sulfate and evaporated under reduced pressure to afford methyl N2-(tert-butoxycarbonyl)-N5-methyl-N5-phenyl-L-glutaminate (3).

### Methyl N5-methyl-N5-phenyl-L-glutaminate hydrochloride (4)

To a stirred solution of methyl N2-(tert-butoxycarbonyl)-N5-methyl-N5-phenyl-L-glutaminate (3) (1.4 g, 3.977 mmol) in 1, 4-dioxane (10 mL) was added 4N HCl in dioxane (10 mL) with drop wise at 0° C. and the reaction mixture was stirred at RT for 2 h. The progress of the reaction was monitored by TLC. After 2 h, reaction mixture was evaporated under reduced pressure to obtained crude compound. The resulting crude was triturated with diethyl ether to afford methyl N5-methyl-N5-phenyl-L-glutaminate hydrochloride (4).

### Methyl N2-((S)-2-((((3-chlorobenzyl) oxy) carbonyl) amino)-3-cyclohexylpropanoyl)-N5-methyl-N5-phenyl-L-glutaminate (5)

At 0° C., to a stirred solution of (S)-2-((((3-chlorobenzyl)oxy)carbonyl)amino)-3-cyclohexylpropanoic acid (1 g, 2.949 mmol) in DMF (10 mL) was added EDC.HCl (845 mg, 4.424 mmol), HOBT (597 mg, 4.424 mmol), DIPEA (1.5 mL, 8.849 mmol) and methyl N5-methyl-N5-phenyl-L-glutaminate hydrochloride (4) (892 mg, 4.424 mmol) and stirred at room temperature for 16 h. The progress of the reaction was monitored by TLC and LCMS. After 16 h, reaction mixture was quenched with ice water (30 mL), extracted with ethyl acetate (2×30 mL), the combined organic layer was dried over sodium sulfate and evaporated under reduced pressure. The crude residue was purified by silica gel column by eluting with 80% ethyl acetate in pet ether to afford methyl N2-((S)-2-((((3-chlorobenzyl)oxy)carbonyl)amino)-3-cyclohexylpropanoyl)-N5-methyl-N5-phenyl-L-glutaminate (5).

### 3-Chlorobenzyl ((S)-3-cyclohexyl-1-(((S)-1-hydroxy-5-(methyl (phenyl) amino)-5-oxopentan-2-yl) amino)-1-oxopropan-2-yl) carbamate (6)

To a stirred solution of methyl N2-((S)-2-((((3-chlorobenzyl)oxy)carbonyl)amino)-3-cyclohexylpropanoyl)-N5-methyl-N5-phenyl-L-glutaminate (5) (1.5 g, 2.62 mmol) in DCM (10 mL) was added 2M LiBH₄ in THE (1.7 mL, 2.62 mmol) at 0° C. and the reaction mixture stirred for 2 h at 0° C. The progress of the reaction was monitored by TLC and LCMS. After 2 h, reaction mixture was quenched with water (20 mL) and extracted with DCM (2×30 mL). Organic layer was washed with brine solution (30 mL), dried over Na₂SO₄ and concentrated to afford 3-chlorobenzyl ((S)-3-cyclohexyl-1-(((S)-1-hydroxy-5-(methyl (phenyl) amino)-5-oxopentan-2-yl) amino)-1-oxopropan-2-yl) carbamate (6).

### 3-Chlorobenzyl ((S)-3-cyclohexyl-1-(((S)-5-(methyl (phenyl) amino)-1, 5-dioxopentan-2-yl) amino)-1-oxopropan-2-yl) carbamate A19

To a stirred solution of 3-chlorobenzyl ((S)-3-cyclohexyl-1-(((S)-1-hydroxy-5-(methyl (phenyl) amino)-5-oxopentan-2-yl) amino)-1-oxopropan-2-yl) carbamate (6) (200 mg, 0.368 mmol) in DCM (5 mL) was added Dess-Martin periodinane (325 mg, 0.736 mmol) at 0° C. and stirred at RT for 3 h. The progress of the reaction was monitored by TLC and LCMS. Reaction mixture was diluted with DCM and was washed with sat. NaHCO₃ solution (3×20 mL) followed by sat. Hypo solution (3×20 mL). Organic layer was dried over anhydrous Na₂SO₄, filtered and concentrated to get crude compound. The crude compound was purified by prep HPLC to afford 3-chlorobenzyl ((S)-3-cyclohexyl-1-(((S)-5-(methyl (phenyl) amino)-1, 5-dioxopentan-2-yl) amino)-1-oxopropan-2-yl) carbamate A19.

TLC system: 10% Methanol in DCM; Rf: 0.5

LCMS (ESI): m/z 542.36 (M+H)⁺

### 3-Chlorobenzyl ((2S)-3-cyclohexyl-1-(((2S)-1-(diethoxyphosphoryl)-1-hydroxy-5-(methyl (phenyl) amino)-5-oxopentan-2-yl) amino)-1-oxopropan-2-yl) carbamate A14

To a stirred solution of 3-chlorobenzyl ((S)-3-cyclohexyl-1-(((S)-5-(methyl (phenyl) amino)-1, 5-dioxopentan-2-yl) amino)-1-oxopropan-2-yl) carbamate A19 (200 mg crude, 0.368 mmol) in DCM (10 mL) was added DIPEA (0.18 mL, 1.104 mmol) followed by added diethylphosphite (0.19 mL, 1.104 mmol) and the reaction mixture stirred at RT for 16 h. The progress of the reaction was monitored by TLC and LCMS. After 16 h, the reaction mixture quenched with ammonium chloride (15 mL) and extracted with DCM (2×15 mL). Combined organic layer was dried over anhydrous Na₂SO₄, and evaporated to afford crude residue. It was purified prep HPLC to afford 3-chlorobenzyl ((2S)-3-cyclohexyl-1-(((2S)-1-(diethoxyphosphoryl)-1-hydroxy-5-(methyl (phenyl) amino)-5-oxopentan-2-yl) amino)-1-oxopropan-2-yl) carbamate A14.

TLC system: 10% MeOH in DCM; R_{f}: 0.45

LCMS (ESI): m/z 680.2 (M+H)⁺

### Synthesis of Compounds A16 and A100

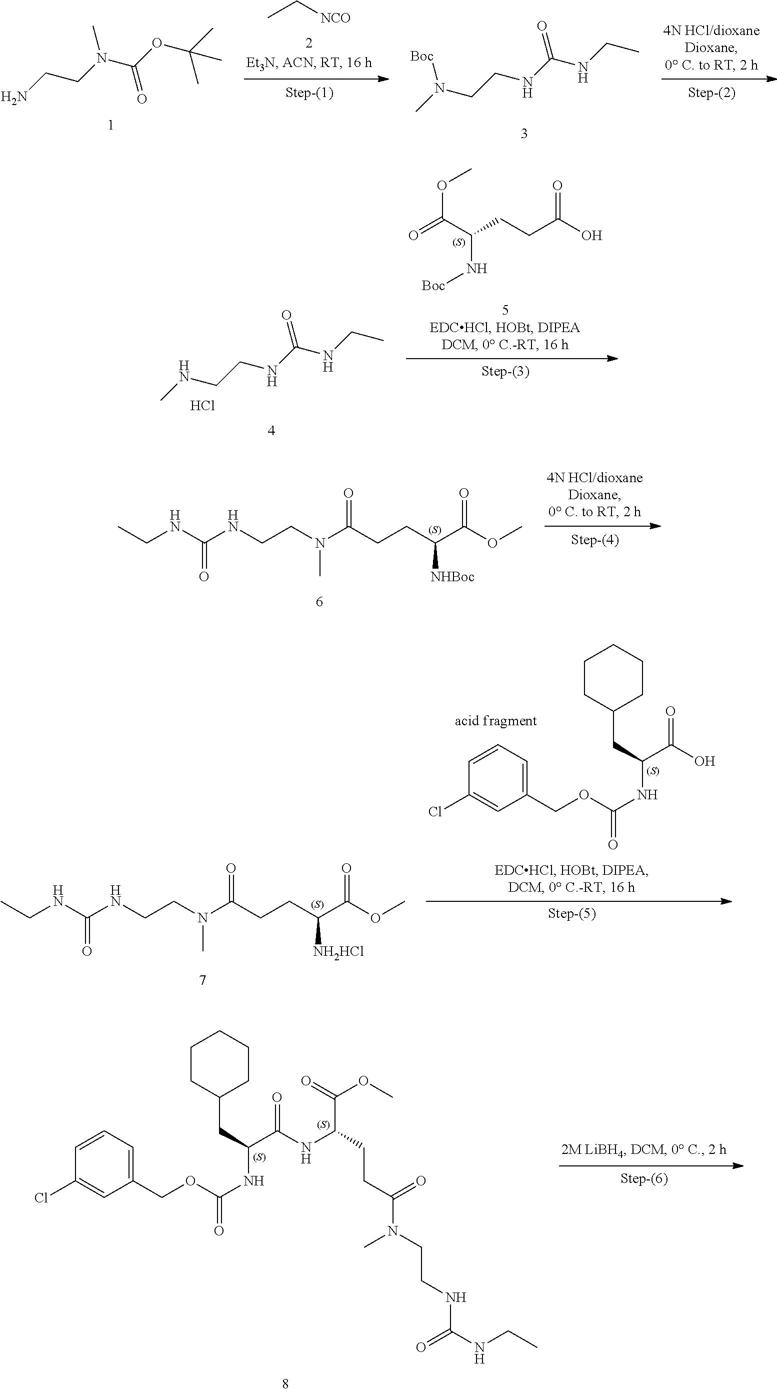

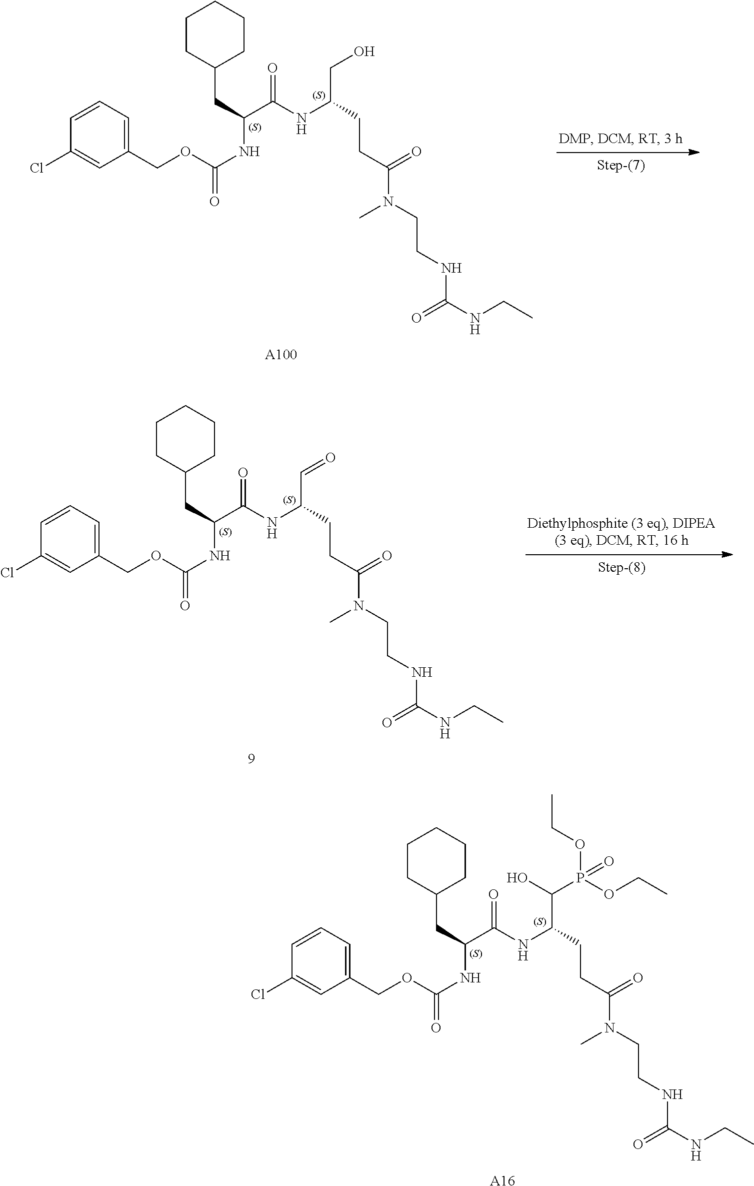

### Tert-Butyl (2-(3-ethylureido)ethyl)(methyl)carbamate (3)

To a stirred solution of tert-butyl (2-aminoethyl)(methyl)carbamate (1) (3 g, 17.22 mmol) in ACN (30 mL), were added triethylamine (4.9 mL, 34.43 mmol), isocyanatoethane (1.64 mL, 20.66 mmol) at 0° C. and stirred at RT for 16 h. After consumption of starting material, the reaction mixture was quenched with water (50 mL) and extracted with ethyl acetate (2×50 mL). The combine organic layer was washed with water (30 mL) and brine (50 mL) dried over anhydrous Na₂SO₄, evaporated under reduced pressure to afford crude compound tert-butyl (2-(3-ethylureido)ethyl)(methyl)carbamate (3).

### 1-Ethyl-3-(2-(methylamino)ethyl)urea hydrogen chloride (4)

To a stirred solution of tert-butyl (2-(3-ethylureido)ethyl)(methyl)carbamate (3) (3 g, 12.24 mmol) in 1,4-dioxane (10 mL) was added 4N HCl in dioxane (15 mL) with drop wise at 0° C. Reaction mixture was stirred at RT for 2 h. The progress of the reaction was monitored by TLC. After consumption of starting material, the reaction mixture was evaporated under reduced pressure to obtain crude compound. Resulting crude triturated with diethyl ether to afford 1-ethyl-3-(2-(methylamino)ethyl)urea hydrochloride (4).

### Methyl N2-(tert-butoxycarbonyl)-N5-(2-(3-ethylureido)ethyl)-N5-methyl-L-glutaminate (6)

To a stirred solution of (S)-4-((tert-butoxycarbonyl)amino)-5-methoxy-5-oxopentanoic acid (5) (3.4 g, 13.186 mmol) DMF (30 mL) added EDC.HCl (4.72 g, 24.724 mmol), HOBT (3.33 g, 24.724 mmol), DIPEA (9 mL, 49.449 mmol) and 1-ethyl-3-(2-(methylamino)ethyl)urea hydrogen chloride (4) (3 g, 16.483 mmol) at 0° C. simultaneously and stirred at room temperature for 16 h. Reaction mixture was diluted with ice water (50 mL), extracted with ethyl acetate (2×50 mL), dried over sodium sulfate and evaporated under reduced pressure. The crude residue was purified by grace NP, compound eluted at 80% Ethyl acetate in pet ether to afford methyl N2-(tert-butoxycarbonyl)-N5-(2-(3-ethylureido)ethyl)-N5-methyl-L-glutaminate (6).

### Methyl N5-(2-(3-ethylureido)ethyl)-N5-methyl-L-glutaminate hydrochloride (7)

To a stirred solution of methyl N2-(tert-butoxycarbonyl)-N5-(2-(3-ethylureido)ethyl)-N5-methyl-L-glutaminate (6) (1.8 g, 4.639 mmol) in 1,4-dioxane (10 mL) was added 4M HCl in dioxane (10 mL) with drop wise at 0° C. Reaction mixture was stirred at RT for 2 h. The progress of the reaction was monitored by TLC. After consumption of starting material, the reaction mixture was evaporated under reduced pressure to obtain crude compound, the resulting crude triturated with diethyl ether to afford methyl N5-(2-(3-ethylureido)ethyl)-N5-methyl-L-glutaminate hydrochloride (7).

### Methyl N2-((S)-2-((((3-chlorobenzyl)oxy)carbonyl)amino)-3-cyclohexylpropanoyl)-N5-(2-(3-ethylureido)ethyl)-N5-methyl-L-glutaminate (8)

To a stirred solution of (S)-2-((((3-chlorobenzyl)oxy)carbonyl)amino)-3-cyclohexylpropanoic acid (1 g, 2.949 mmol) DMF (10 mL), added EDC.HCl (0.84 g, 4.42 mmol), HOBt (0.59 g, 4.42 mmol), DIPEA (1.6 mL, 8.847 mmol) and methyl N5-(2-(3-ethylureido)ethyl)-N5-methyl-L-glutaminate hydrochloride (7) (1.14 g, 3.538 mmol) at 0° C. and reaction mixture was stirred at room temperature for 16 h. The progress of the reaction was monitored by TLC and LCMS. After 16 h, the reaction mixture was diluted with ice water (50 mL) and extracted with ethyl acetate (2×50 mL). Organic layer was dried over sodium sulfate and evaporated under reduced pressure. The crude residue was purified by grace NP, compound eluted at 10% methanol in dichloromethane to afford methyl N2-((S)-2-((((3-chlorobenzyl)oxy)carbonyl)amino)-3-cyclohexylpropanoyl)-N5-(2-(3-ethylureido)ethyl)-N5-methyl-L-glutaminate (8).

### 3-Chlorobenzyl ((12S,15S)-16-cyclohexyl-12-(hydroxymethyl)-8-methyl-4,9,14-trioxo-3,5,8,13-tetraazahexadecan-15-yl)carbamate A100

To a stirred solution of methyl N2-((S)-2-((((3-chlorobenzyl)oxy)carbonyl)amino)-3-cyclohexylpropanoyl)-N5-(2-(3-ethylureido)ethyl)-N5-methyl-L-glutaminate (8) (0.800 g, 1.313 mmol) in DCM (16 mL) was added 2M LiBH₄ in THE (1.9 mL, 3.94 mmol) at 0° C., reaction mixture stirred for 2 h at 0° C. The progress of the reaction was monitored by TLC and LCMS. Then the reaction mixture was quenched with water (20 mL) and extracted with DCM (2×20 mL). Organic layer was washed with brine solution (20 mL), dried over Na₂SO₄ and concentrated to get crude. Crude compound was purified by normal phase chromatography to afford pure 3-chlorobenzyl ((12S,15S)-16-cyclohexyl-12-(hydroxymethyl)-8-methyl-4,9,14-trioxo-3,5,8,13-tetraazahexadecan-15-yl)carbamate A100.

TLC system: 10% Methanol in DCM; Rf: 0.2

LCMS (ESI): m/z 582.55 (M+H)⁺

### 3-Chlorobenzyl ((12S,15S)-16-cyclohexyl-12-formyl-8-methyl-4,9,14-trioxo-3,5,8,13-tetraazahexadecan-15-yl)carbamate (9)

To a stirred solution of 3-chlorobenzyl ((12S,155S)-16-cyclohexyl-12-(hydroxymethyl)-8-methyl-4,9,14-trioxo-3,5,8,13-tetraazahexadecan-15-yl)carbamate A100 (200 mg, 0.344 mmol) in DCM (10 mL) was added Dess-Martin periodinane (291 mg, 0.688 mmol) at 0° C. and stirred at RT for 3 h. The progress of the reaction was monitored by TLC and LCMS. Reaction mixture was diluted with DCM (15 mL) and washed with sat. hypo solution (3×20 mL), sat. NaHCO₃ solution (3×20 mL). Organic layer was dried over anhydrous Na₂SO₄, filtered and concentrated to afford 3-chlorobenzyl((12S,15S)-16-cyclohexyl-12-formyl-8-methyl-4,9,14-trioxo-3,5,8,13-tetraazahexadecan-15-yl)carbamate (9).

### 3-Chlorobenzyl ((12S,15S)-16-cyclohexyl-12-((diethoxyphosphoryl)(hydroxy)methyl)-8-methyl-4,9,14-trioxo-3,5,8,13-tetraazahexadecan-15-yl)carbamate A16

To a stirred solution of 3-chlorobenzyl ((12S,15S)-16-cyclohexyl-12-formyl-8-methyl-4,9,14-trioxo-3,5,8,13-tetraazahexadecan-15-yl)carbamate (9) (250 mg, 0.4317 mmol) was dissolved in DCM (10 mL), were added Et₃N (0.23 mL, 1.29 mmol) and diethyl phosphite (178 mg, 1.29 mmol) at 0° C. and stirred at RT for 16 h. The progress of the reaction was monitored by TLC and LCMS. After 16 h, the reaction mixture was diluted H₂O (15 mL) and extracted with DCM (2×15 mL). The organic layer was dried over anhydrous Na₂SO₄, and filtered, crude compound purified by prep-HPLC afford 3-chlorobenzyl ((12S,15S)-16-cyclohexyl-12-((diethoxyphosphoryl)(hydroxy)methyl)-8-methyl-4,9,14-trioxo-3,5,8,13-tetraazahexadecan-15-yl)carbamate A16.

TLC system: 10% Methanol in DCM; Rf: 0.3

LCMS (ESI): m/z 718.52 (M+H)⁺

### Synthesis of Compounds A27 and A17

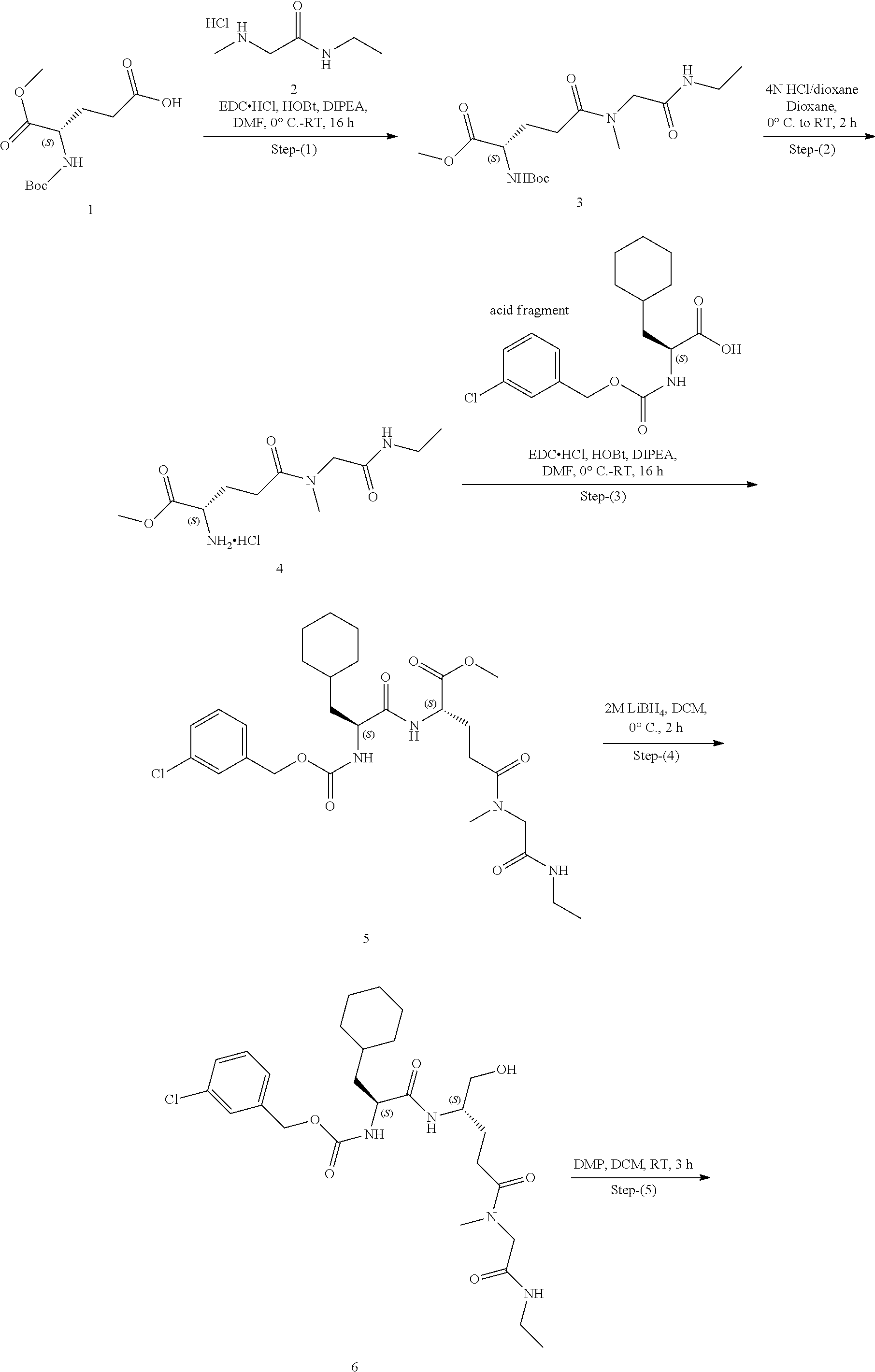

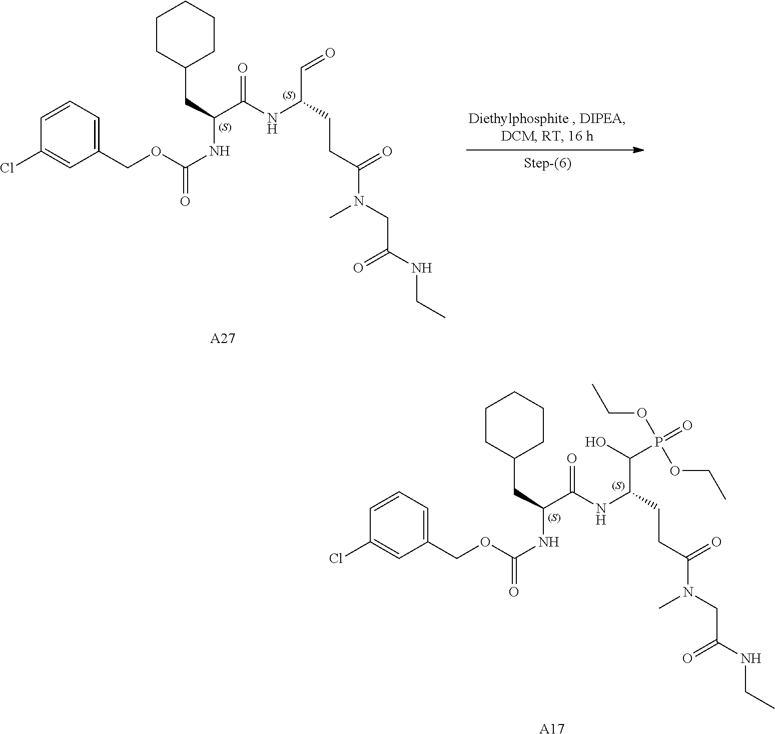

### Methyl N2-(tert-butoxycarbonyl)-N5-(2-(ethylamino)-2-oxoethyl)-N5-methyl-L-glutaminate (3)

To a stirred solution of (S)-4-((tert-butoxycarbonyl)amino)-5-methoxy-5-oxopentanoic acid (1) (1.0 g, 3.83 mmol) DMF (10 mL) added EDC.HCl (1.1 g, 5.74 mmol), HOBT (775 mg, 5.74 mmol), DIPEA (2.1 mL, 11.49 mmol) at 0° C. Then reaction mass was stirred for 15 min and then added N-ethyl-2-(methylamino)acetamide (2) (533 mg, 4.59 mmol) at 0° C. and stirred at room temperature for 16 h. The progress of the reaction was monitored by TLC. Reaction mixture was quenched with ice water (100 mL) and extracted with ethyl acetate (2×60 mL), combined organic layers were washed with brine solution (30 mL), dried over sodium sulfate and evaporated under reduced pressure. The crude residue was purified by normal phase chromatography with eluted 3% methanol in dichloromethane to afford methyl N2-(tert-butoxycarbonyl)-N5-(2-(ethylamino)-2-oxoethyl)-N5-methyl-L-glutaminate (3).

### Methyl N5-(2-(ethylamino)-2-oxoethyl)-N5-methyl-L-glutaminate hydrochloride (4)

To a stirred solution of methyl N2-(tert-butoxycarbonyl)-N5-(2-(ethylamino)-2-oxoethyl)-N5-methyl-L-glutaminate (3) (1.1 g, 3.06 mmol) in 1,4-dioxane (11 mL) was added 4 M HCl in dioxane (11 mL) with drop wise at 0° C. and the reaction mixture was stirred at RT for 3 h. The progress of the reaction was monitored by TLC. After 3 h, the reaction mixture was evaporated to dryness under reduced pressure to obtain crude compound, the resulting crude triturated with diethyl ether to afford methyl N5-(2-(ethylamino)-2-oxoethyl)-N5-methyl-L-glutaminate hydrochloride (4). This product was used to next step without further purification.

### Methyl N2-((S)-2-((((3-chlorobenzyl) oxy) carbonyl) amino)-3-cyclohexylpropanoyl)-N5-(2-(ethylamino)-2-oxoethyl)-N5-methyl-L-glutaminate (5)

To a stirred solution of (S)-2-((((3-chlorobenzyl)oxy)carbonyl)amino)-3-cyclohexylpropanoic acid (acid fragment) (1.0 g, 2.95 mmol) (synthesis reported in Compd-17 final report) in DMF (10 mL) added EDC.HCl (845 mg, 4.42 mmol), HOBT (596 mg, 4.42 mmol), DIPEA (1.6 mL, 8.85 mmol) at 0° C. Then reaction mass was stirred for 15 min was added methyl N5-(2-(ethylamino)-2-oxoethyl)-N5-methyl-L-glutaminate hydrochloride (870 mg, 2.95 mmol) at 0° C. stirred at room temperature for 16 h. The progress of the reaction was monitored by TLC and LCMS. After 16 h, the reaction mixture was quenched with ice water (60 mL) and extracted with ethyl acetate (2×100 mL). The organic layer was dried over sodium sulfate and evaporated under reduced pressure. The crude residue was purified by normal phase chromatography with eluted 5% methanol in dichloromethane to afford methyl N2-((S)-2-((((3-chlorobenzyl) oxy) carbonyl) amino)-3-cyclohexylpropanoyl)-N5-(2-(ethylamino)-2-oxoethyl)-N5-methyl-L-glutaminate (5).

### 3-Chlorobenzyl ((S)-3-cyclohexyl-1-(((S)-5-((2-(ethylamino)-2-oxoethyl) (methyl) amino)-1-hydroxy-5-oxopentan-2-yl) amino)-1-oxopropan-2-yl) carbonate (6)

To a stirred solution of methyl N2-((S)-2-((((3-chlorobenzyl)oxy)carbonyl)amino)-3-cyclohexylpropanoyl)-N5-(2-(ethylamino)-2-oxoethyl)-N5-methyl-L-glutaminate (5) (1.0 g, 1.72 mmol) in DCM (20 mL) was added 2M LiBH₄ in THE (2.5 mL, 5.17 mmol) at 0° C. reaction mixture stirred for 2 h at 0° C. The progress of the reaction was monitored by TLC and LCMS. The reaction mixture was quenched with water (20 mL) and extracted with DCM (2×20 mL). The combined organic layer was washed with brine solution (20 mL) and the organic layer was dried over Na₂SO₄ and concentrated to get crude compound. Crude compound was purified by normal phase chromatography to afford 3-chlorobenzyl ((S)-3-cyclohexyl-1-(((S)-5-((2-(ethylamino)-2-oxoethyl) (methyl) amino)-1-hydroxy-5-oxopentan-2-yl) amino)-1-oxopropan-2-yl) carbonate (6).

### 3-Chlorobenzyl ((S)-3-cyclohexyl-1-(((S)-5-((2-(ethylamino)-2-oxoethyl) (methyl) amino)-1,5-dioxopentan-2-yl)amino)-1-oxopropan-2-yl)carbamate A27

To a stirred solution of 3-chlorobenzyl ((S)-3-cyclohexyl-1-(((S)-5-((2-(ethylamino)-2-oxoethyl)(methyl)amino)-1-hydroxy-5-oxopentan-2-yl)amino)-1-oxopropan-2-yl)carbamate (6) (100 mg, 0.18 mmol) was dissolved in DCM (2 mL) was added Dess-Martin periodinane (153 mg, 0.36 mmol) at 0° C. and stirred at RT for 3 h. The progress of the reaction was monitored by TLC and LCMS. After 3 h, the reaction mixture was diluted with DCM (15 mL) and washed with sat. Hypo solution (3×20 mL) and sat. NaHCO₃ solution (3×20 mL). Organic layer was dried over anhydrous Na₂SO₄, filtered and concentrated to get crude compound, this crude was purified by prep HPLC to afford 3-chlorobenzyl((S)-3-cyclohexyl-1-(((S)-5-((2-(ethylamino)-2-oxoethyl)(methyl)amino)-1,5-dioxopentan-2-yl)amino)-1-oxopropan-2-yl)carbamate A27.

TLC system: 10% Methanol in DCM; Rf: 0.4

LCMS (ESI): m/z 551.2 (M+H)⁺

### 3-Chlorobenzyl ((2S)-3-cyclohexyl-1-(((2S)-1-(diethoxyphosphoryl)-5-((2-(ethylamino)-2-oxoethyl) (methyl) amino)-1-hydroxy-5-oxopentan-2-yl) amino)-1-oxopropan-2-yl) carbamate A17

To a stirred solution of 3-chlorobenzyl ((S)-3-cyclohexyl-1-(((S)-5-((2-(ethylamino)-2-oxoethyl)(methyl)amino)-1,5-dioxopentan-2-yl)amino)-1-oxopropan-2-yl)carbamate A27 (200 mg, 0.36 mmol) in DCM (5 mL) added DIPEA (0.23 mL, 1.09 mmol) followed by diethyl phosphite (0.13 mL, 1.09 mmol) at 0° C. then reaction mixture stirred at RT for 16 h. The progress of the reaction was monitored by TLC and LCMS. After 16 h, the reaction mixture quenched with water (15 mL) and extracted with DCM (2×20 mL). Combined organic layer was dried over anhydrous Na₂SO₄, and evaporated to afford crude residue. It was purified by prep HPLC to afford 3-chlorobenzyl((2S)-3-cyclohexyl-1-(((2S)-1-(diethoxyphosphoryl)-5-((2-(ethylamino)-2-oxoethyl) (methyl)amino)-1-hydroxy-5-oxopentan-2-yl) amino)-1-oxopropan-2-yl) carbamate A17.

TLC system: 10% Methanol in DCM; Rf: 0.4

LCMS (ESI): m/z 689.46 (M+H)⁺

### Synthesis of Compounds A46, A28, A21, and A101

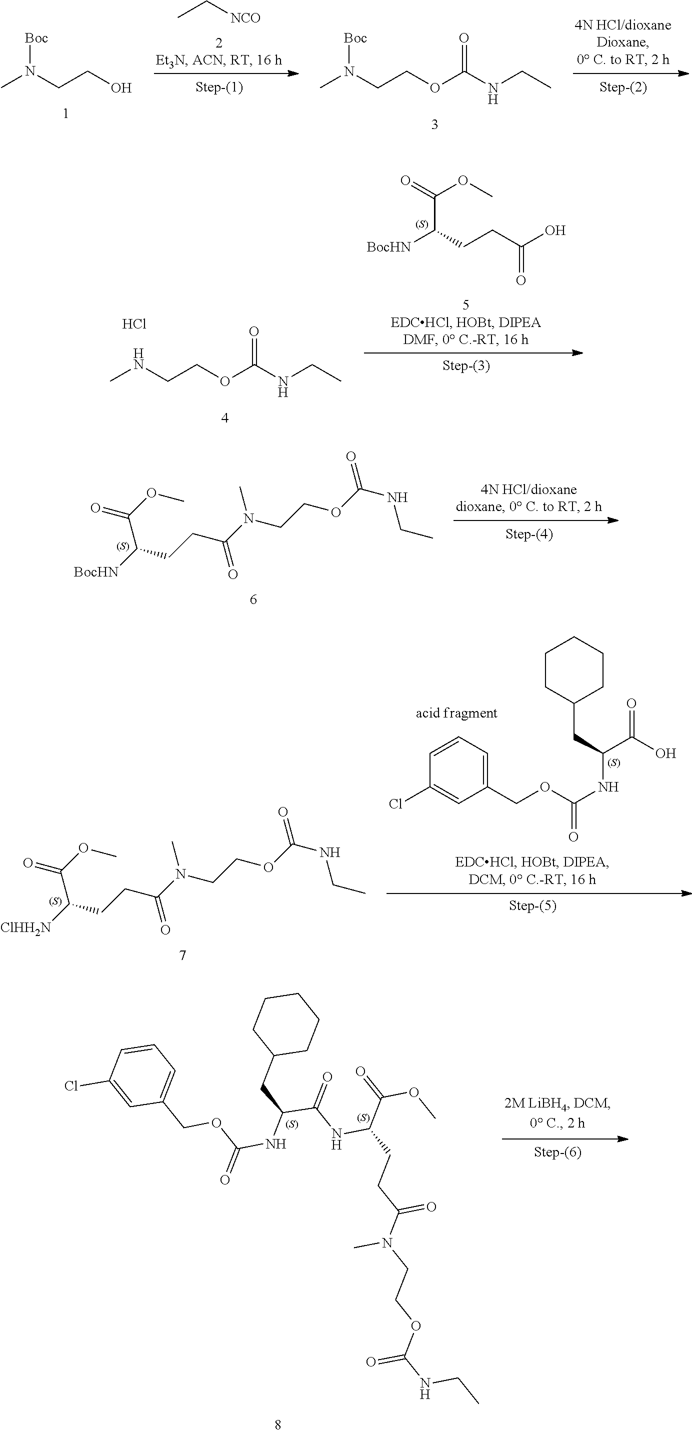

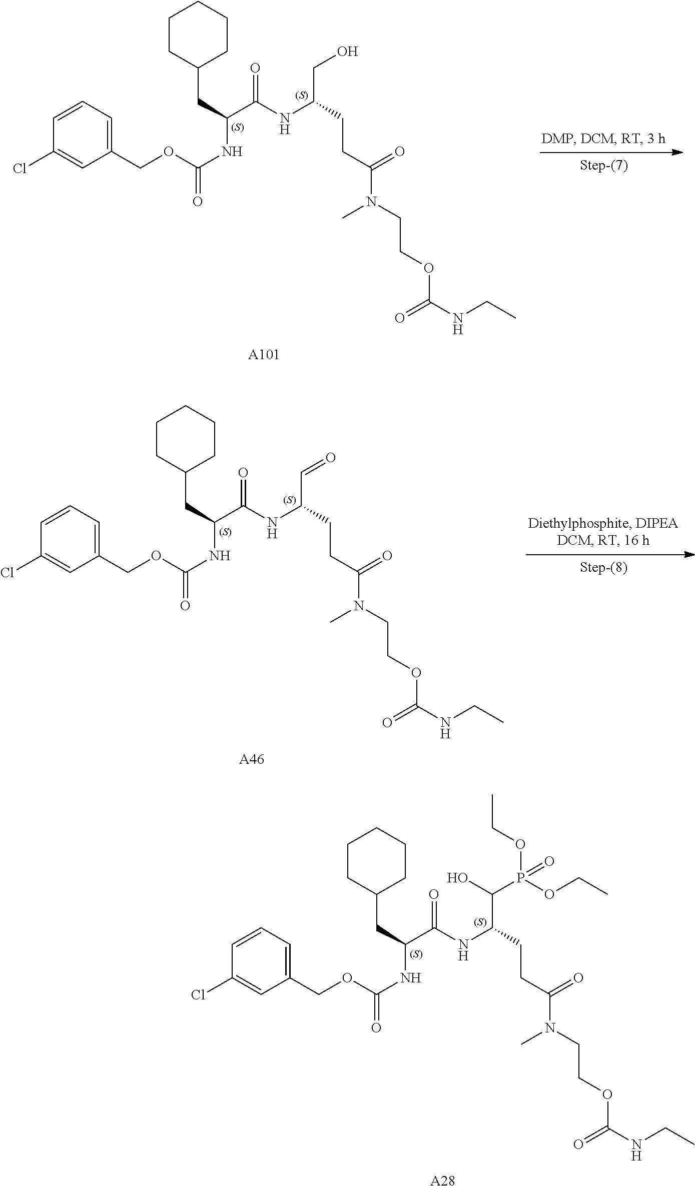

### Tert-Butyl (2-((ethylcarbamoyl)oxy)ethyl)(methyl)carbamate (3)

To a stirred solution of tert-butyl (2-hydroxyethyl)(methyl)carbamate (1) (2 g, 11.42 mmol) in ACN (20 mL), were added triethyl amine (3.29 mL, 22.85 mmol), isocyanatoethane (1.08 mL, 13.714 mmol) at 0° C. and stirred at RT for 16 h. After consumption of starting material, the reaction mixture was quenched with water (50 mL) and extracted with DCM (2×50 mL). Organic layer was washed with water (30 mL) and brine (50 mL), dried over anhydrous Na₂SO₄ and evaporated under reduced pressure to afford tert-butyl (2-((ethylcarbamoyl)oxy)ethyl)(methyl)carbamate (3).

### 2-(Methylamino)ethyl ethylcarbamate hydrogen chloride (4)

To a stirred solution of tert-butyl (2-((ethylcarbamoyl)oxy)ethyl)(methyl)carbamate (3) (2 g, 8.13 mmol) in 1,4-dioxane (20 mL) was added 4N HCl in dioxane (20 mL) dropwise at 0° C. Reaction mixture was stirred at RT for 2 h. The progress of the reaction was monitored by TLC. After consumption of starting material, the reaction mixture was evaporated under reduced pressure to obtain crude compound. Resulting crude triturated with diethyl ether to afford 2-(methylamino)ethyl ethylcarbamate hydrochloride (4.

### Methyl N2-(tert-butoxycarbonyl)-N5-(2-((ethylcarbamoyl)oxy)ethyl)-N5-methyl-L-glutaminate (6)

To a stirred solution of (S)-4-((tert-butoxycarbonyl)amino)-5-methoxy-5-oxopentanoic acid (5) (2 g, 7.662 mmol) DMF (20 mL) were added EDC.HCl (2.19 g, 11.49 mmol), HOBt (1.55 g, 11.494 mmol), DIPEA (4.2 mL, 22.98 mmol) and 2-(methylamino)ethyl ethylcarbamate hydrochloride (4) (1.67 g, 9.195 mmol) at 0° C. and reaction mixture was stirred at room temperature for 16 h. Reaction mixture was diluted with ice water (50 mL) and extracted with ethyl acetate (2×50 mL). Organic layer was dried over sodium sulfate and evaporated under reduced pressure. The crude residue was purified by grace NP, compound eluted at 80% ethyl acetate in pet ether to afford methyl N2-(tert-butoxycarbonyl)-N5-(2-((ethylcarbamoyl)-oxy)ethyl)-N5-methyl-L-glutaminate (6).

### Methyl N5-(2-((ethylcarbamoyl)oxy)ethyl)-N5-methyl-L-glutaminate hydrochloride (7)

To a stirred solution of methyl N2-(tert-butoxycarbonyl)-N5-(2-(3-ethylureido)ethyl)-N5-methyl-L-glutaminate (6) (2 g, 5.14 mmol) in 1,4-dioxane (10 mL) was added drop wise 4 M HCl in dioxane (10 mL) at 0° C. Reaction mixture was stirred at RT for 2 h. The progress of the reaction was monitored by TLC. After consumption of starting material, the reaction mixture was evaporated under reduced pressure to obtain crude compound. Resulting crude was triturated with diethyl ether to afford methyl N5-(2-((ethylcarbamoyl)oxy)ethyl)-N5-methyl-L-glutaminate hydrochloride (7).

### Methyl N2-((S)-2-((((3-chlorobenzyl)oxy)carbonyl)amino)-3-cyclohexylpropanoyl)-N5-(2-((ethylcarbamoyl)oxy)ethyl)-N5-methyl-L-glutaminate (8)

To a stirred solution of (S)-2-((((3-chlorobenzyl)oxy)carbonyl)amino)-3-cyclohexylpropanoic acid (1 g, 2.95 mmol) DMF (10 mL) were added EDC.HCl (0.84 g, 4.42 mmol), HOBt (0.59 g, 4.42 mmol), DIPEA (1.6 mL, 8.847 mmol) and methyl N5-(2-((ethylcarbamoyl)oxy)ethyl)-N5-methyl-L-glutaminate hydrochloride (7) (1.15 g, 3.53 mmol) at 0° C. and reaction mixture was stirred at room temperature for 16 h. Reaction mixture was diluted with ice water (30 mL) and extracted with ethyl acetate (2×30 mL). Organic layer was dried over sodium sulfate and evaporated under reduced pressure. The crude residue was purified by grace NP, compound eluted at 10% methanol in dichloromethane to afford methyl N2-((S)-2-((((3-chlorobenzyl)oxy)carbonyl)amino)-3-cyclohexylpropanoyl)-N5-(2-((ethylcarbamoyl)oxy)ethyl)-N5-methyl-L-glutaminate (8).

### 3-Chlorobenzyl ((12S,15S)-16-cyclohexyl-12-(hydroxymethyl)-8-methyl-4,9,14-trioxo-5-oxa-3,8,13-triazahexadecan-15-yl)carbamate A101

To a stirred solution of methyl N2-((S)-2-((((3-chlorobenzyl)oxy)carbonyl)amino)-3-cyclohexylpropanoyl)-N5-(2-((ethylcarbamoyl)oxy)ethyl)-N5-methyl-L-glutaminate (8) (0.100 g, 0.163 mmol) in DCM (5 mL) was added 2M LiBH₄ in THE (0.25 mL, 0.492 mmol) at 0° C. and reaction mixture was stirred for 2 h at 0° C. Reaction mixture was quenched with water (10 mL) and extracted with DCM (2×10 mL). Organic layer was washed with brine solution (10 mL), dried over Na₂SO₄ and concentrated. Resulting crude was purified by prep-HPLC to afford 3-chlorobenzyl ((12S,15S)-16-cyclohexyl-12-(hydroxymethyl)-8-methyl-4,9,14-trioxo-5-oxa-3,8,13-triazahexadecan-15-yl)carbamate A101.

TLC system: 10% Methanol in DCM; Rf: 0.2

LCMS (ESI): m/z 583.50 (M+H)⁺

### 3-Chlorobenzyl ((12S, 15S)-16-cyclohexyl-12-formyl-8-methyl-4, 9,14-trioxo-5-oxa-3,8,13-triazahexadecan-15-yl)carbamate A46

To a stirred solution of 3-chlorobenzyl ((12S,15S)-16-cyclohexyl-12-(hydroxymethyl)-8-methyl-4,9,14-trioxo-5-oxa-3,8,13-triazahexadecan-15-yl)carbamate A101 (350 mg, 0.601 mmol) was dissolved in DCM (10 mL) was added Dess-Martin periodinane (764 mg, 1.804 mmol) at 0° C. and stirred at RT for 3 h. The progress of the reaction was monitored by TLC and LCMS. Reaction mixture was diluted with DCM (10 mL) and washed with sat. Hypo solution (3×15 mL), sat. NaHCO₃ solution (3×15 mL). Organic layer was dried over anhydrous Na₂SO₄, filtered and concentrated to get crude compound. The crude compound was purified by prep HPLC to afford 3-chlorobenzyl ((12S, 15S)-16-cyclohexyl-12-formyl-8-methyl-4,9,14-trioxo-5-oxa-3,8,13-triazahexadecan-15-yl)carbamate A46.

TLC system: 70% EtOAC in hexane; Rf: 0.6

LCMS (ESI): m/z 581.60 (M+H)⁺

### 3-Chlorobenzyl ((12S,15S)-16-cyclohexyl-12-((diethoxyphosphoryl)(hydroxy)methyl)-8-methyl-4,9,14-trioxo-5-oxa-3,8,13-triazahexadecan-15-yl)carbamate A28

To a stirred solution of 3-chlorobenzyl ((12,155S)-16-cyclohexyl-12-formyl-8-methyl-4,9,14-trioxo-5-oxa-3,8,13-triazahexadecan-15-yl)carbamate A46 (300 mg, 0.517 mmol) in DCM (10 mL) were added DIPEA (0.3 mL, 1.55 mmol) and diethyl phosphite (214 mg, 1.55 mmol) at 0° C. and reaction mixture was stirred at RT for 16 h. Reaction mixture was diluted with H₂O (15 mL) and extracted with DCM (2×15 mL). Organic layer was dried over anhydrous Na₂SO₄ and evaporated. Resulting crude was purified by prep-HPLC to afford 3-chlorobenzyl ((12S,15S)-16-cyclohexyl-12-((diethoxyphosphoryl)(hydroxy)methyl)-8-methyl-4,9,14-trioxo-5-oxa-3,8,13-triazahexadecan-15-yl)carbamate A28.

TLC system: 10% Methanol in DCM; Rf: 0.3

LCMS (ESI): m/z 719.54 (M+H)⁺

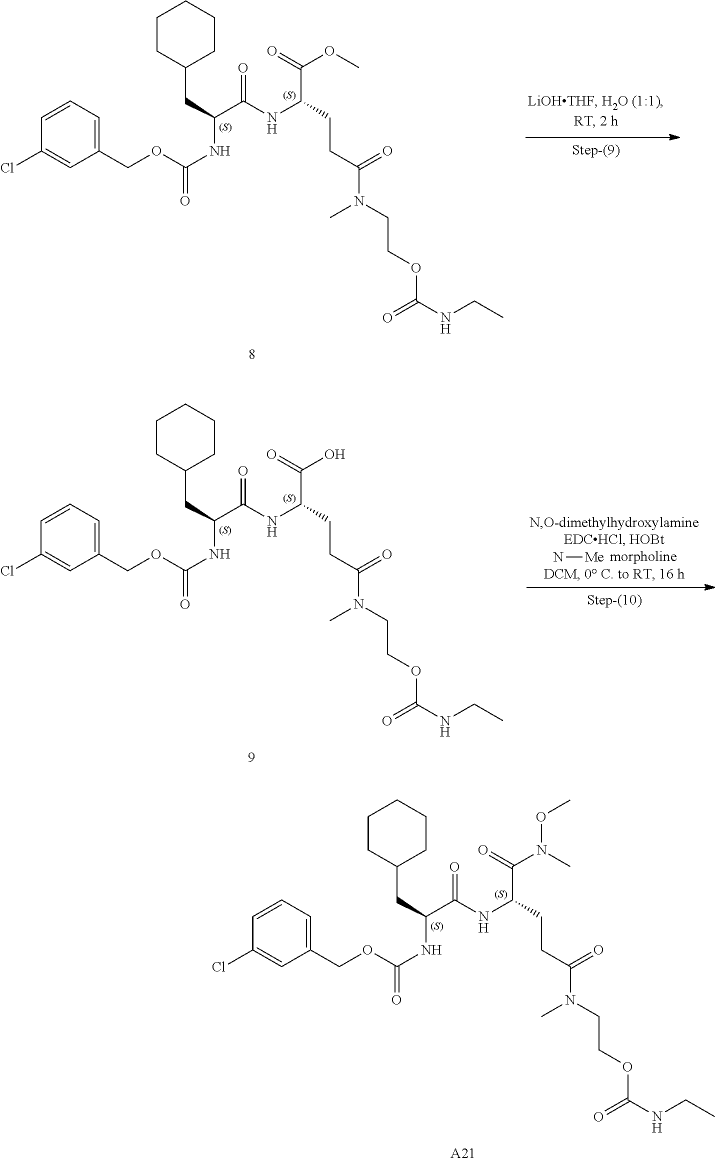

### N2-((S)-2-((((3-Chlorobenzyl)oxy)carbonyl)amino)-3-cyclohexylpropanoyl)-N5-(2-((ethylcarbamoyl)oxy)ethyl)-N5-methyl-L-glutamine (9)

To a stirred solution of methyl N2-((S)-2-((((3-chlorobenzyl)oxy)carbonyl)amino)-3-cyclohexylpropanoyl)-N5-(2-((ethylcarbamoyl)oxy)ethyl)-N5-methyl-L-glutaminate (8) (300 mg, 0.49 mmol) in THE (3 mL), methanol (2 mL) and water (1 mL) was added lithium hydroxide (0.023 g, 0.98 mmol) at RT and stirred at RT for 2 h. Solvent was evaporated under reduced pressure, crude compound was acidified with 2 N aq. HCl solution to pH ˜4, and extracted with ethyl acetate (2×10 mL). Organic layer dried over sodium sulfate, concentrated under reduced pressure to afford N2-((S)-2-((((3-chlorobenzyl)oxy)-carbonyl)amino)-3-cyclohexylpropanoyl)-N5-(2-((ethylcarbamoyl)oxy)ethyl)-N5-methyl-L-glutamine (9).

### 3-Chlorobenzyl ((12S,15S)-16-cyclohexyl-12-(methoxy(methyl)carbamoyl)-8-methyl-4,9,14-trioxo-5-oxa-3,8,13-triazahexadecan-15-yl)carbamate A21

To a stirred solution of N2-((S)-2-((((3-chlorobenzyl)oxy)carbonyl)amino)-3-cyclohexylpropanoyl)-N5-(2-((ethylcarbamoyl)oxy)ethyl)-N5-methyl-L-glutamine (9) (250 mg, 0.419 mmol) in tetrahydrofuran (5 mL) and DCM (5 mL) were added EDC.HCl (96 mg, 0.503 mmol), HOBt (67 mg, 0.503 mmol), N-methylmorpholine (50 mg, 0.503 mmol) and N,O-dimethylhydroxylamine (48 mg, 0.503 mmol) at 0° C. and reaction mixture was stirred at room temperature for 16 h. Reaction mixture was diluted with ice water (20 mL) and extracted with ethyl acetate (2×20 mL). Organic layer was dried over sodium sulfate and evaporated under reduced pressure. Resulting crude was purified by prep-HPLC to afford 3-chlorobenzyl ((12S,15S)-16-cyclohexyl-12-(methoxy(methyl)carbamoyl)-8-methyl-4,9,14-trioxo-5-oxa-3,8,13-triazahexadecan-15-yl)carbamate A21.

TLC system: 10% Methanol in DCM; Rf: 0.3

LCMS (ESI): m/z 640.60 (M+H)⁺

### Synthesis of A44, A31, and A32

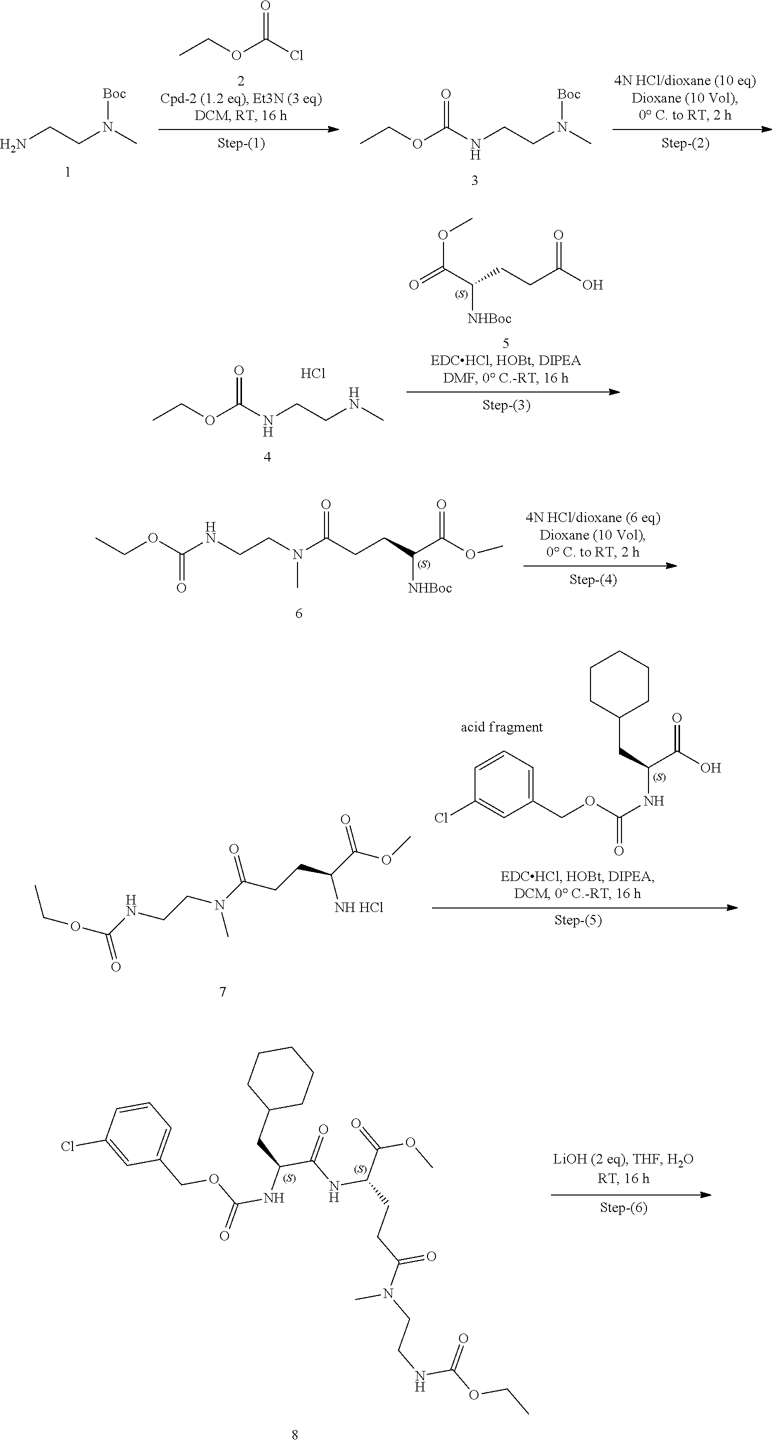

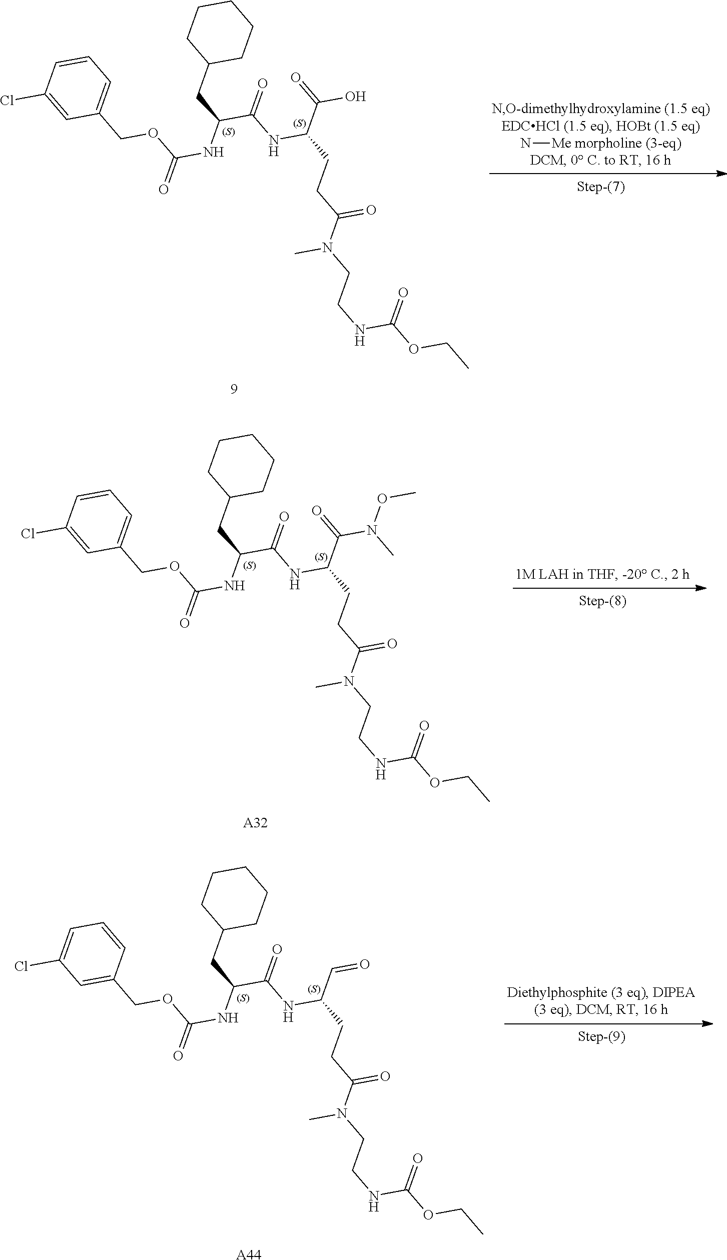

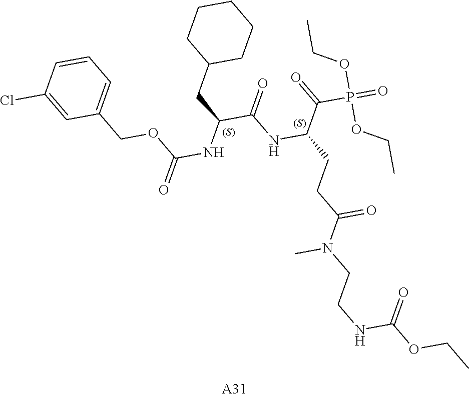

### Tert-butyl (2-((ethoxycarbonyl)amino)ethyl)(methyl)carbamate (3)

To a stirred solution of tert-butyl (2-aminoethyl)(methyl)carbamate (1) (3 g, 17.241 mmol) in DCM (30 mL) were added Et₃N (7.1 mL, 51.723 mmol) and ethyl carbonochloridate (2) (2.2 g, 20.689 mmol) at 0° C. drop wise simultaneously, and the reaction mixture was stirred at RT for 16 h. The progress of the reaction was monitored by TLC. The reaction mixture was diluted with ice water (50 mL), extracted with ethyl acetate (2×50 mL). Combined organic layers were washed with brine solution (30 mL), dried over sodium sulfate and evaporated under reduced pressure to get crude residue. Crude was purified by combi-flash, compound eluted at 10% Methanol in DCM to afford tert-butyl (2-((ethoxycarbonyl)amino)ethyl)(methyl)carbamate (3).

### Ethyl (2-(methylamino)ethyl)carbamate (4)

To a stirred solution tert-butyl (2-((ethoxycarbonyl)amino)ethyl)(methyl)carbamate (3) (1.8 g, 7.31 mmol) in 1,4-dioxane (20 mL) at 0° C. was added drop wise 4N HCl/dioxane (10 mL) and the reaction mixture was stirred at RT for 2 h. The progress of the reaction was monitored by TLC. After consumption of starting material, the reaction mixture was evaporated under reduced pressure to obtained crude compound, the resulting crude triturated with diethyl ether to afford ethyl (2-(methylamino)ethyl)carbamate (4).

### Methyl N2-(tert-butoxycarbonyl)-N5-(2-((ethoxycarbonyl)amino)ethyl)-N5-methyl-D-glutaminate (6)

To a stirred solution of ethyl (2-(methylamino)ethyl)carbamate (4) (2 g, 7.6628 mmol) in DMF (20 mL) were added EDC.HCl (2.1 g, 11.49 mmol), HOBT (1.5 g, 11.4942 mmol), DIPEA (2.6 mL, 22.98 mmol) and ethyl (2-(methylamino)ethyl)carbamate (2) (2.2 g, 9.19 mmol) at 0° C. and the reaction mixture was stirred at RT for 16 h. The progress of the reaction was monitored by TLC and LCMS. After 16 h, the reaction mixture was diluted with ice water (50 mL), extracted with ethyl acetate (2×50 mL). Combined organic layers were washed with brine solution (30 mL), dried over sodium sulfate and evaporated under reduced pressure to get crude residue. Crude was purified by combi-flash compound eluted at 10% Methanol in DCM to afford methyl N2-(tert-butoxycarbonyl)-N5-(2-((ethoxycarbonyl)amino)ethyl)-N5-methyl-L-glutaminate (3).

### Methyl N5-(2-((ethoxycarbonyl)amino)ethyl)-N5-methyl-L-glutaminate hydrochloride (7)

To a stirred solution of methyl N2-(tert-butoxycarbonyl)-N5-(2-((ethoxycarbonyl)amino)ethyl)-N5-methyl-L-glutaminate (6) (0.4 g, 1.028 mmol) in 1,4-dioxane (5 mL) was added 4N HCl in dioxane (5 mL) with drop wise at 0° C. and the reaction mixture was stirred at RT for 2 h. After consumption of starting material, the reaction mixture was evaporated under reduced pressure to obtained crude compound, the resulting crude triturated with diethyl ether to afford methyl N5-(2-((ethoxycarbonyl)amino)ethyl)-N5-methyl-L-glutaminate hydrochloride (7).

### Methyl N2-((S)-2-((((3-chlorobenzyl)oxy)carbonyl)amino)-3-cyclohexylpropanoyl)-N5-(2-((ethoxycarbonyl)amino)ethyl)-N5-methyl-L-glutaminate (8)

To a stirred solution of (S)-2-((((3-chlorobenzyl)oxy)carbonyl)amino)-3-cyclohexylpropanoic acid (Acid fragment) (1.2 g, 3.53 mmol) in DMF (12 mL) was added EDC.HCl (1.01 g, 5.30 mmol), HOBT (0.718 g, 5.30 mmol), DIPEA (1.2 mL, 10.61 mmol) and methyl N5-(2-((ethoxycarbonyl)amino)ethyl)-N5-methyl-L-glutaminate hydrochloride (7) (1.2 g, 4.24 mmol) at 0° C. simultaneously and stirred at room temperature for 16 h. After 16 h, reaction mixture was diluted with ice water (50 mL), extracted with ethyl acetate (2×50 mL). Combined organic layers were washed with brine solution (30 mL), dried over sodium sulfate and evaporated under reduced pressure to get crude residue. Crude was purified by combi-flash, compound eluted at 10% methanol in DCM to afford methyl N2-((S)-2-((((3-chlorobenzyl)oxy)carbonyl)amino)-3-cyclohexylpropanoyl)-N5-(2-((ethoxycarbonyl)amino)ethyl)-N5-methyl-L-glutaminate 8.

### N2-((S)-2-((((3-chlorobenzyl)oxy)carbonyl)amino)-3-cyclohexylpropanoyl)-N5-(2-((ethoxycarbonyl)amino)ethyl)-N5-methyl-L-glutamine (9)

To a stirred solution of methyl N2-((S)-2-((((3-chlorobenzyl)oxy)carbonyl)amino)-3-cyclohexylpropanoyl)-N5-(2-((ethoxycarbonyl)amino)ethyl)-N5-methyl-L-glutaminate (8) (1.5 g, 2.459 mmol) in THE (20 mL) and water (10 mL), was added lithium hydroxide (177 mg, 7.3770 mmol) at RT and stirred at room temperature for 3 h. The progress of the reaction was monitored by TLC and LCMS. After 3 h, the reaction mixture completely distilled under reduced pressure, crude compound acidified with 1N HCL solution up to pH ˜4, and extracted with ethyl acetate (2×10 mL), dried over sodium sulfate, concentrated under reduced pressure to afford crude N2-((S)-2-((((3-chlorobenzyl)oxy)carbonyl)amino)-3-cyclohexylpropanoyl)-N5-(2-((ethoxycarbonyl)amino)ethyl)-N5-methyl-L-glutamine (9).

### 3-Chlorobenzyl ((12S,15S)-16-cyclohexyl-12-(methoxy(methyl)carbamoyl)-8-methyl-4,9,14-trioxo-3-oxa-5,8,13-triazahexadecan-15-yl)carbamate A32

To a stirred solution of N2-((S)-2-((((3-chlorobenzyl)oxy)carbonyl)amino)-3-cyclohexylpropanoyl)-N5-(2-((ethoxycarbonyl)amino)ethyl)-N5-methyl-L-glutamine (9) (160 mg, 0.2684 mmol) in THE (3 mL) and DCM (3 mL), added EDC.HCl (61 mg, 0.3221 mmol), HOBT (43 mg, 0.3221 mmol), N-methylmorpholine (0.1 mL, 0.3221 mmol) and N,O-dimethylhydroxylamine (31 mg, 0.3221 mmol) at 0° C. simultaneously and stirred at room temperature for 16 h. The progress of the reaction was monitored by TLC and LCMS. Then the reaction mixture was diluted with ice water (15 mL), extracted with ethyl acetate (2×10 mL). Combined organic layers were washed with brine solution (30 mL), dried over sodium sulfate and evaporated under reduced pressure to get crude residue. Crude was purified by prep-HPLC purification afforded 3-chlorobenzyl ((12S,15S)-16-cyclohexyl-12-(methoxy(methyl)carbamoyl)-8-methyl-4,9,14-trioxo-3-oxa-5,8,13-triazahexadecan-15-yl)carbamate A32.

TLC system: 10% Methanol in DCM; Rf: 0.6

LCMS (ESI): m/z 640.67 (M+H)

### Ethyl ((5S,8S)-1-(3-chlorophenyl)-5-(cyclohexylmethyl)-8-formyl-12-methyl-3,6,11-trioxo-2-oxa-4,7,12-triazatetradecan-14-yl)carbamate A44

To a stirred solution of 3-chlorobenzyl ((12S,15S)-16-cyclohexyl-12-(methoxy(methyl)carbamoyl)-8-methyl-4,9,14-trioxo-3-oxa-5,8,13-triazahexadecan-15-yl)carbamate A32 (140 mg, 0.2187 mmol) in THE (2 mL) was added 2M LiAlH₄ in THE (0.2 mL, 0.6562 mmol) at −20° C. and reaction mixture was stirred at −20° C. for 2 h. The progress of the reaction was monitored by TLC and LCMS. After consumption of starting material, the reaction mixture was diluted with ice water (10 mL) extracted with ethyl acetate (2×10 mL), organic layer was washed with water (2×10 mL), and brine (10 mL). The combined organic layer was dried over sodium sulfate, filtered and evaporated under reduced pressure obtained crude. It was purified by prep-HPLC to ethyl ((5S,8S)-1-(3-chlorophenyl)-5-(cyclohexylmethyl)-8-formyl-12-methyl-3,6,11-trioxo-2-oxa-4,7,12-triazatetradecan-14-yl)carbamate A44.

TLC system: 10% Methanol in DCM; Rf: 0.3

LCMS (ESI): m/z 581.59 (M+H)⁺

### 3-Chlorobenzyl ((12S,15S)-16-cyclohexyl-12-((diethoxyphosphoryl)(hydroxy)methyl)-8-methyl-4,9,14-trioxo-3-oxa-5,8,13-triazahexadecan-15-yl)carbamate A31

To a stirred solution of ethyl ((5S,8S)-1-(3-chlorophenyl)-5-(cyclohexylmethyl)-8-formyl-12-methyl-3,6,11-trioxo-2-oxa-4,7,12-triazatetradecan-14-yl)carbamate A44 (200 mg, 0.3448 mmol) in DCM (2 mL) added DIPEA (0.2 mL, 1.0344 mmol) followed by added diethyl phosphite (0.1 mL, 1.0344 mmol) and the reaction mixture stirred at RT for 16 h. The progress of the reaction was monitored by TLC and LCMS. After 16 h, the reaction mixture diluted with ice water (15 mL) and extracted with DCM (2×20 mL). Combined organic layers were washed with brine solution (10 mL), dried over sodium sulfate and evaporated under reduced pressure to get crude residue. Crude was purified by purified prep HPLC to afford 3-chlorobenzyl ((12S,15S)-16-cyclohexyl-12-((diethoxyphosphoryl)(hydroxy)methyl)-8-methyl-4,9,14-trioxo-3-oxa-5,8,13-triazahexadecan-15-yl)carbamate A31.

TLC system: 10% Methanol in DCM; Rf: 0.3

LCMS (ESI): m/z 719.71 (M+H)⁺

### Synthesis of Compounds A33, A34, and A24

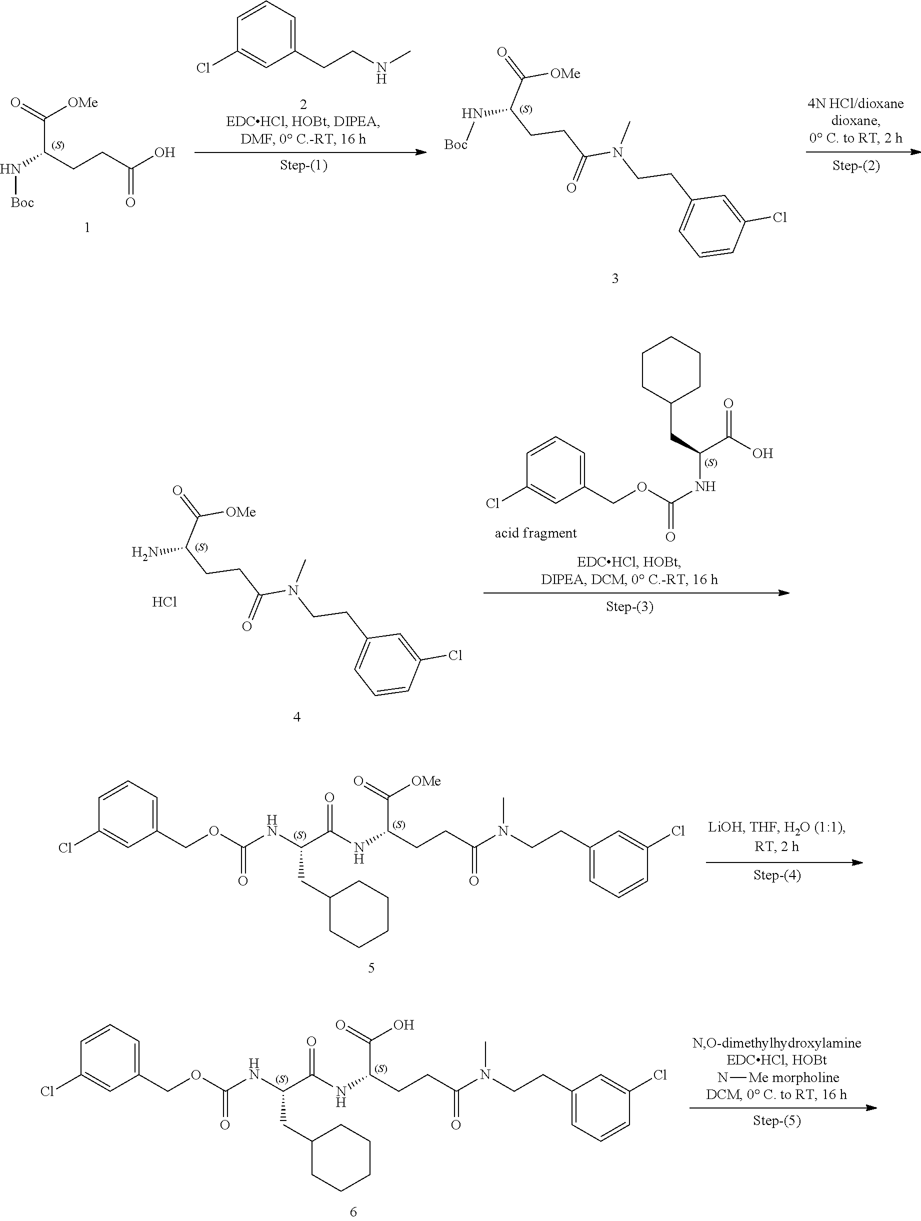

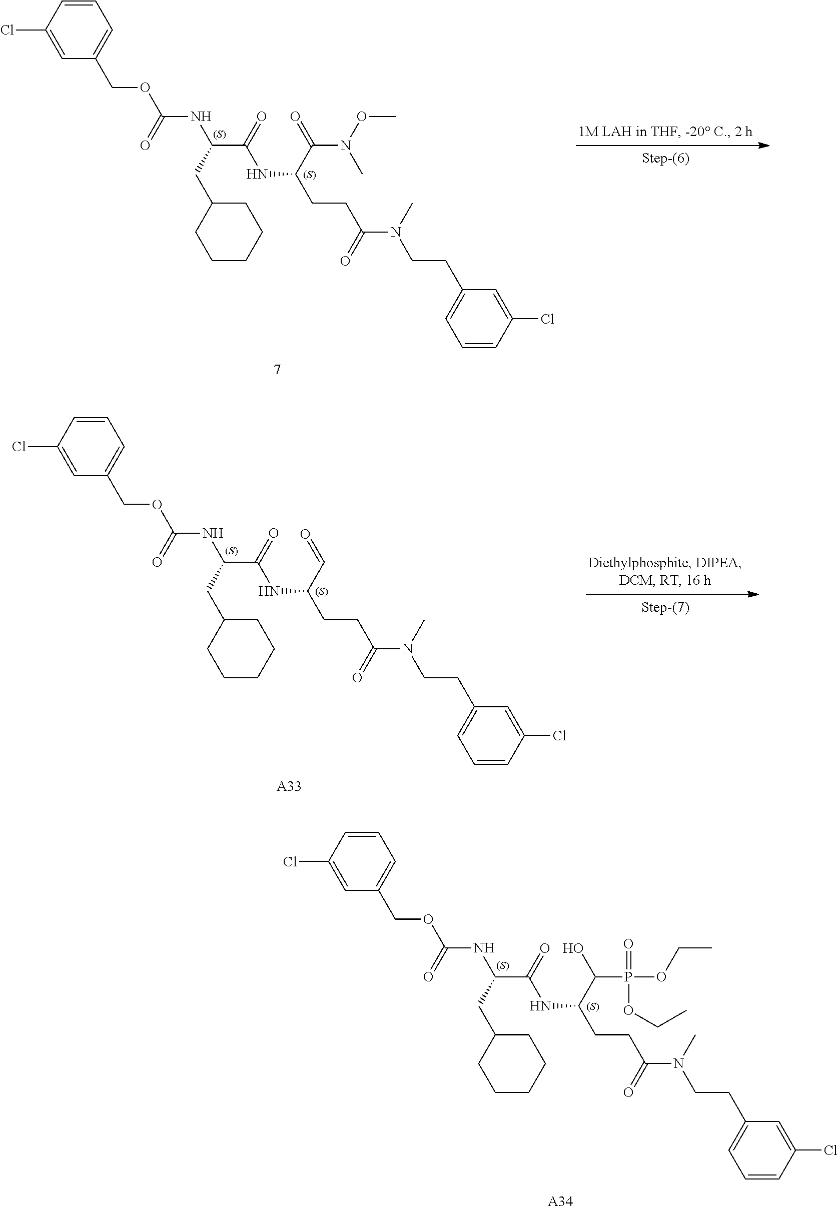

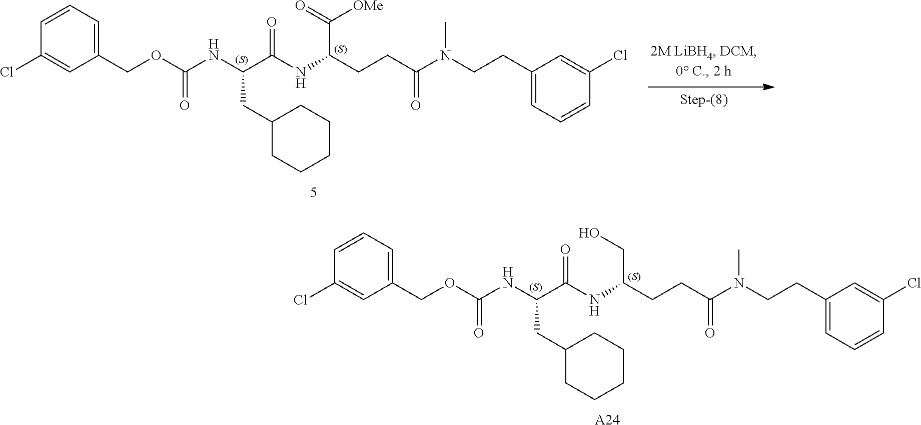

### Methyl N2-(tert-butoxycarbonyl)-N5-(3-chlorophenethyl)-N5-methyl-L-glutaminate (3)

To a stirred solution of (S)-4-((tert-butoxycarbonyl)amino)-5-methoxy-5-oxopentanoic acid (1) (3 g, 11.4942 mmol) in DMF (30 mL) were added EDC.HCl (3.29 g, 17.2413 mmol), HOBT (2.32 g, 17.2413 mmol), DIPEA (6.3 mL, 34.4827 mmol) and 2-(3-chlorophenyl)-N-methylethan-1-amine (2) (2.71 g, 13.7931 mmol) at 0° C. and the reaction mixture was stirred at RT for 16 h. The progress of the reaction was monitored by TLC and LCMS. After 16 h, the reaction mixture was diluted with ice water (50 mL), extracted with ethyl acetate (2×50 mL). The combined organic layer was dried over sodium sulfate, filtered and evaporated under reduced pressure. The crude residue was purified by combi-flash compound eluted at 60% Ethyl acetate in pet. ether to afford methyl N2-(tert-butoxycarbonyl)-N5-(3-chlorophenethyl)-N5-methyl-L-glutaminate (3).

### Methyl N5-(3-chlorophenethyl)-N5-methyl-L-glutaminate hydrochloride (4)

To a stirred solution of methyl N2-(tert-butoxycarbonyl)-N5-(3-chlorophenethyl)-N5-methyl-L-glutaminate (3) (2.2 g, 5.339 mmol) in 1,4-dioxane (20 mL) was added 4N HCl in dioxane (25 mL) with drop wise at 0° C. and the reaction mixture was stirred at RT for 2 h. The progress of the reaction was monitored by TLC and LCMS. After consumption of starting material, the reaction mixture was evaporated under reduced pressure to obtained crude compound, the resulting crude triturated with diethyl ether to afford methyl N5-(3-chlorophenethyl)-N5-methyl-L-glutaminate hydrochloride (4).

### Methyl N2-((S)-2-((((3-chlorobenzyl)oxy)carbonyl)amino)-3-cyclohexylpropanoyl)-N5-(3-chlorophenethyl)-N5-methyl-L-glutaminate (5)

To a stirred solution of (S)-2-((((3-chlorobenzyl)oxy)carbonyl)amino)-3-cyclohexylpropanoic acid (1.2 g, 3.5502 mmol) DMF (20 mL) added EDC.HCl (1.1 g, 5.3254 mmol), HOBT (0.718 g, 5.3254 mmol), DIPEA (1.96 mL, 10.6508 mmol) and methyl N5-(3-chlorophenethyl)-N5-methyl-L-glutaminate hydrogen chloride (4) (1.66 g, 5.3254 mmol) at 0° C. simultaneously and stirred at room temperature for 16 h. The progress of the reaction was monitored by TLC and LCMS. After 16 h, reaction mixture was diluted with ice water (30 mL), extracted with ethyl acetate (2×30 mL). The combined organic layer was dried over sodium sulfate, filtered and evaporated under reduced pressure. The crude residue was purified by combi-flash, compound eluted at 80% Ethyl acetate in pet ether to afford methyl N2-((S)-2-((((3-chlorobenzyl)oxy)carbonyl)amino)-3-cyclohexylpropanoyl)-N5-(3-chlorophenethyl)-N5-methyl-L-glutaminate (5).

### N2-((S)-2-((((3-chlorobenzyl)oxy)carbonyl)amino)-3-cyclohexylpropanoyl)-N5-(3-chlorophenethyl)-N5-methyl-L-glutamine (6)

To a stirred solution of methyl N2-((S)-2-((((3-chlorobenzyl)oxy)carbonyl)amino)-3-cyclohexylpropanoyl)-N5-(3-chlorophenethyl)-N5-methyl-L-glutaminate (5) (1.8 g, 2.8436 mmol) in THE (20 mL) and water (1 mL), was added lithium hydroxide (196 mg, 8.5308 mmol) at RT and stirred at room temperature for 3 h. The progress of the reaction was monitored by TLC and LCMS. After 3 h, the reaction mixture completely distilled under reduced pressure, crude compound acidified with 2N HCL solution up to pH ˜4, and extracted with ethyl acetate (2×10 mL), dried over sodium sulfate, concentrated under reduced pressure to afford crude compound N2-((S)-2-((((3-chlorobenzyl)oxy)carbonyl)amino)-3-cyclohexylpropanoyl)-N5-(3-chlorophenethyl)-N5-methyl-L-glutamine (6).

### 3-Chlorobenzyl ((S)-1-(((S)-5-((3-chlorophenethyl)(methyl)amino)-1-(methoxy(methyl)amino)-1,5-dioxopentan-2-yl)amino)-3-cyclohexyl-1-oxopropan-2-yl)carbamate (7)

To a stirred solution of N2-((S)-2-((((3-chlorobenzyl)oxy)carbonyl)amino)-3-cyclohexylpropanoyl)-N5-(3-chlorophenethyl)-N5-methyl-L-glutamine (6) (100 mg, 0.1615 mmol) in THE (5 mL) and DCM (5 mL), added EDC.HCl (37 mg, 0.1938 mmol), HOBT (26 mg, 0.1938 mmol), N-methylmorpholine (0.1 mL, 0.1938 mmol) and N,O-dimethylhydroxylamine (18 mg, 0.1938 mmol) at 0° C. simultaneously and stirred at room temperature for 16 h. The progress of the reaction was monitored by TLC and LCMS. Then the reaction mixture was diluted with ice water (10 mL), extracted with ethyl acetate (2×10 mL). The combined organic layer was dried over sodium sulfate, filtered and evaporated under reduced pressure. The crude residue was purified by combi-flash, compound eluted at 70% ethyl acetate in pet ether to afford 3-chlorobenzyl((S)-1-(((S)-5-((3-chlorophenethyl)(methyl)amino)-1-(methoxy(methyl)amino)-1,5-dioxopentan-2-yl)amino)-3-cyclohexyl-1-oxopropan-2-yl)carbamate (7).

TLC system: 10% Methanol in DCM; Rf: 0.6

LCMS (ESI): m/z 663.57 (M+H)⁺

### 3-Chlorobenzyl ((S)-1-(((S)-5-((3-chlorophenethyl)(methyl)amino)-1,5-dioxopentan-2-yl)amino)-3-cyclohexyl-1-oxopropan-2-yl)carbamate A33

To a stirred solution of 3-chlorobenzyl ((S)-1-(((S)-5-((3-chlorophenethyl)(methyl)amino)-1-(methoxy(methyl)amino)-1,5-dioxopentan-2-yl)amino)-3-cyclohexyl-1-oxopropan-2-yl)carbamate(7) (200 mg, 0.3021 mmol) in THE (10 mL) was added 2M LiAlH₄ in THE (0.6 mL, 0.6042 mmol) at −20° C. and reaction mixture was stirred at −20° C. for 2 h. The progress of the reaction was monitored by TLC and LCMS. After consumption of starting material, the reaction mixture was diluted with ice water (10 mL) extracted with ethyl acetate (2×10 mL), organic layer was washed with water (2×10 mL), and brine (10 mL). The combined organic layer was dried over sodium sulfate, filtered and evaporated under reduced pressure. It was purified by prep-HPLC to afforded 3-chlorobenzyl ((S)-1-(((S)-5-((3-chlorophenethyl)(methyl)amino)-1,5-dioxopentan-2-yl)amino)-3-cyclohexyl-1-oxopropan-2-yl)carbamate A33.

TLC system: 10% Methanol in DCM; Rf: 0.3

LCMS (ESI): m/z 604.19 (M+H)⁺

### 3-Chlorobenzyl ((2S)-1-(((2S)-5-((3-chlorophenethyl)(methyl)amino)-1-(diethoxyphosphoryl)-1-hydroxy-5-oxopentan-2-yl)amino)-3-cyclohexyl-1-oxopropan-2-yl)carbamate A34

To a stirred solution of 3-chlorobenzyl ((S)-1-(((S)-5-((3-chlorophenethyl)(methyl)amino)-1,5-dioxopentan-2-yl)amino)-3-cyclohexyl-1-oxopropan-2-yl)carbamate A33 (200 mg, 0.3316 mmol) in DCM (10 mL) added DIPEA (0.18 mL, 0.995 mmol) followed by added diethyl phosphite (0.1 mL, 0.995 mmol) and the reaction mixture stirred at RT for 16 h. The progress of the reaction was monitored by TLC and LCMS. After 16 h, the reaction mixture quenched with ammonium chloride (15 mL) and extracted with DCM (2×20 mL). Combined organic layer was dried over anhydrous Na₂SO₄, and evaporated to afford crude residue. It was purified prep HPLC to afford 3-chlorobenzyl ((2S)-1-(((2S)-5-((3-chlorophenethyl)(methyl)amino)-1-(diethoxyphosphoryl)-1-hydroxy-5-oxopentan-2-yl)amino)-3-cyclohexyl-1-oxopropan-2-yl)carbamate A34.

TLC system: 10% Methanol in DCM; Rf: 0.3

LCMS (ESI): m/z 719.54 (M+H)⁺

### 3-Chlorobenzyl ((S)-1-(((S)-5-((3-chlorophenethyl)(methyl)amino)-1-hydroxy-5-oxopentan-2-yl)amino)-3-cyclohexyl-1-oxopropan-2-yl)carbamate A24

To a stirred solution of methyl N2-((S)-2-((((3-chlorobenzyl)oxy)carbonyl)amino)-3-cyclohexylpropanoyl)-N5-(3-chlorophenethyl)-N5-methyl-L-glutaminate (5) (150 mg, 0.2369 mmol) in DCM (10 mL) was added 2M LiBH₄ in THE (0.23 mL, 0.4739 mmol) at 0° C. and the reaction mixture stirred for 3 h at 0° C. The progress of the reaction was monitored by TLC and LCMS. Then reaction mixture was quenched with water (10 mL) and extracted with DCM (2×10 mL). Organic layer was washed with brine solution, dried over Na₂SO₄ and concentrated to get crude compound. It was purified prep HPLC to afforded 3-chlorobenzyl ((S)-1-(((S)-5-((3-chlorophenethyl)(methyl)amino)-1-hydroxy-5-oxopentan-2-yl)amino)-3-cyclohexyl-1-oxopropan-2-yl)carbamate A24.

TLC system: 10% Methanol in DCM; Rf: 0.1

LCMS (ESI): m/z 606.6 (M+H)⁺

### Synthesis of Compounds A49, A102, and A35

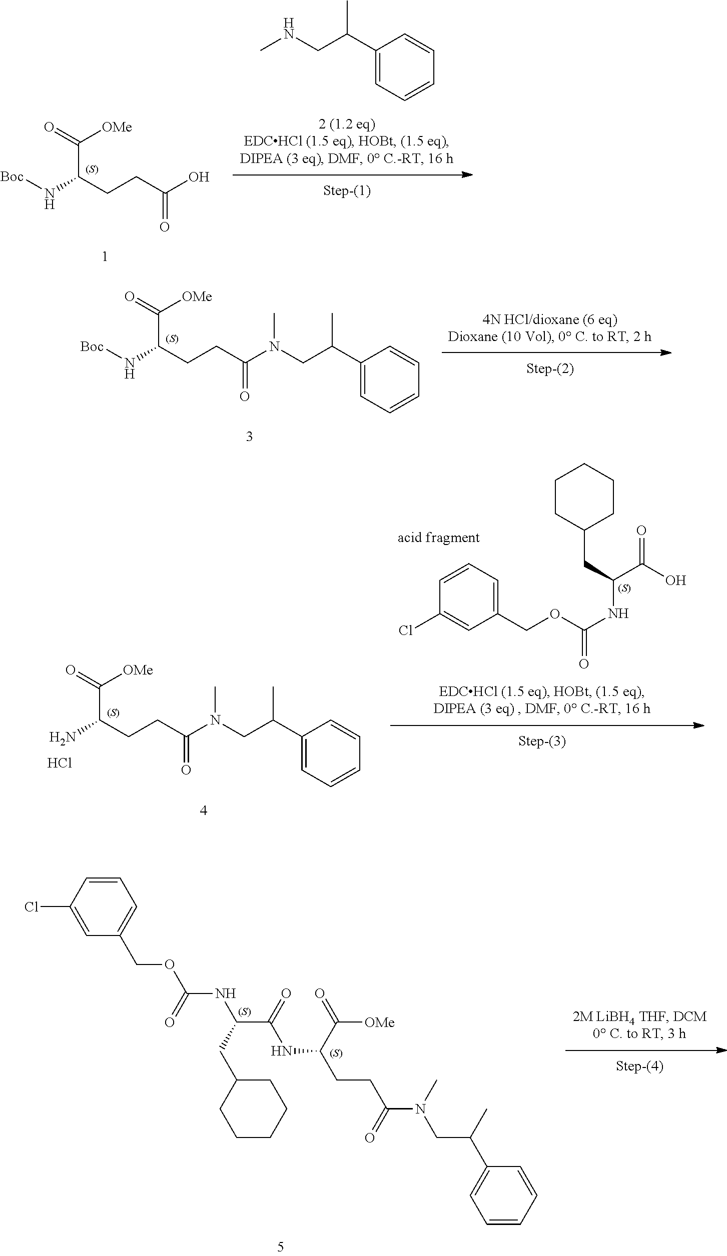

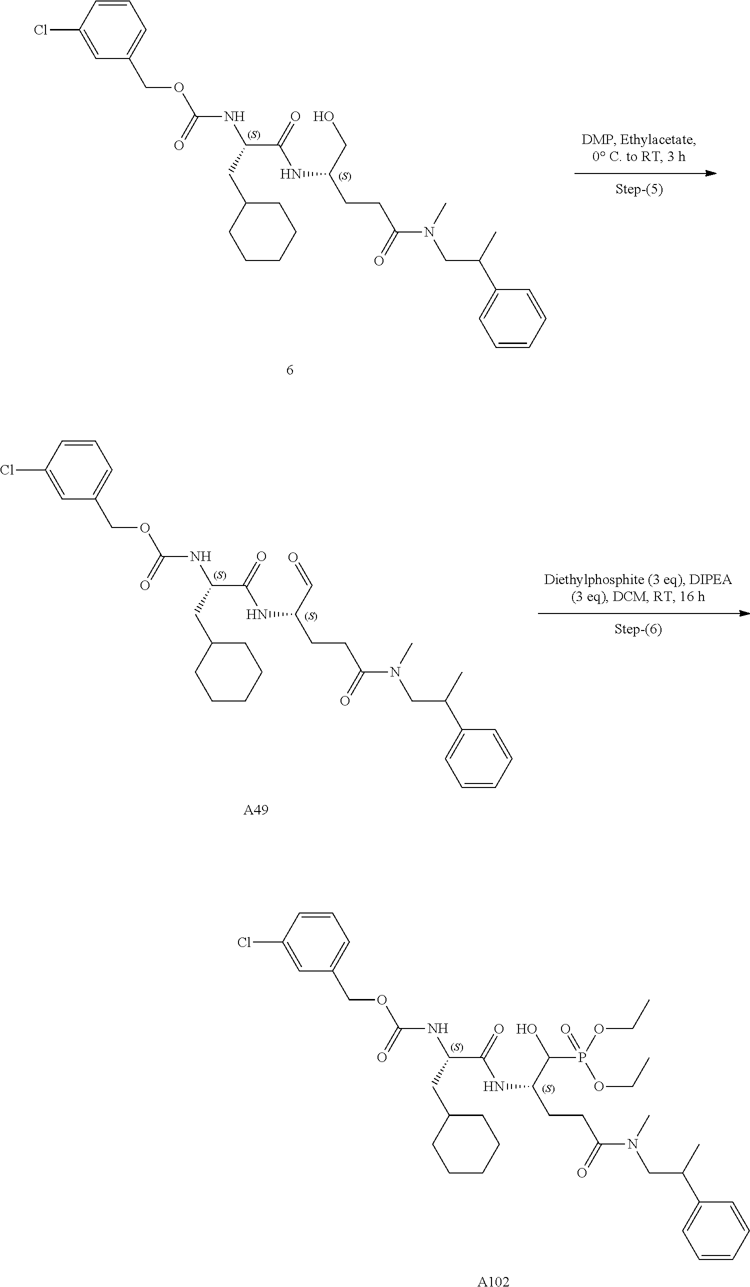

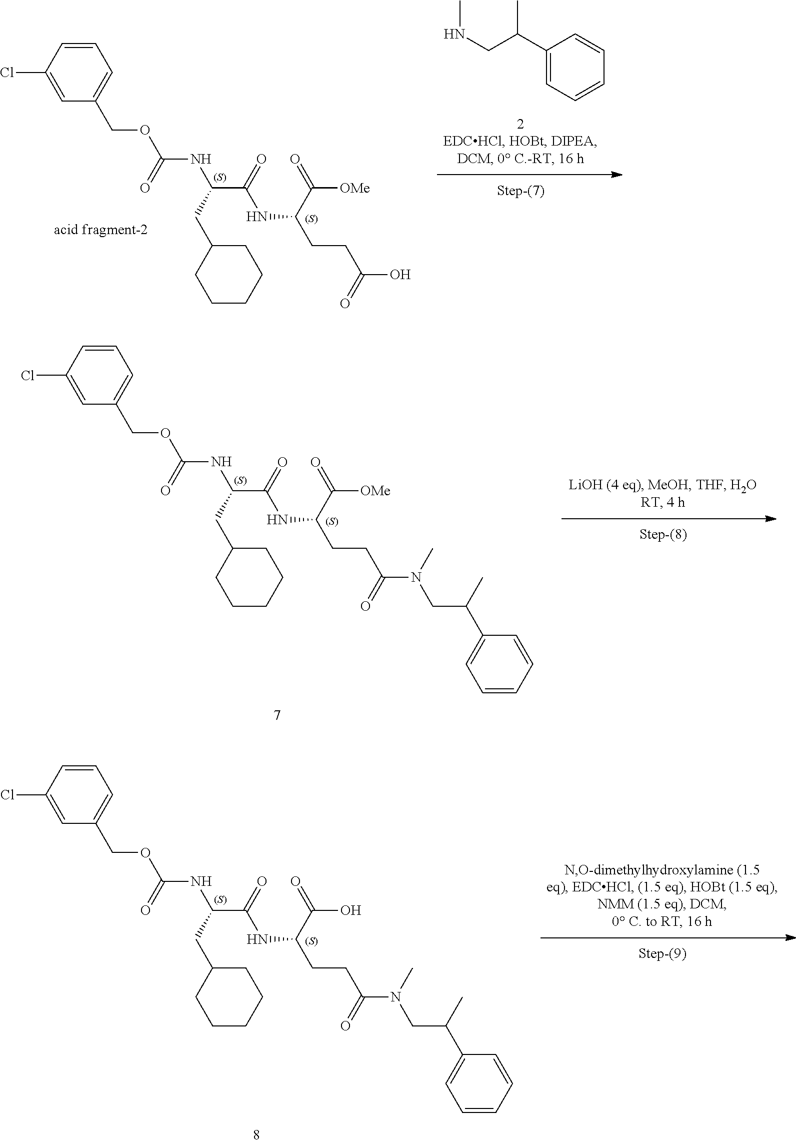

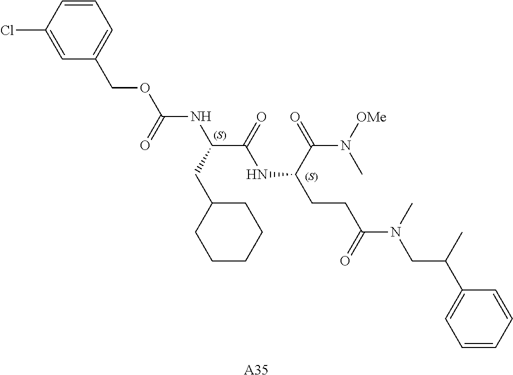

### Methyl N2-(tert-butoxycarbonyl)-N5-(2-(ethylamino)-2-oxoethyl)-N5-methyl-L-glutaminate (3)

To a stirred solution of (S)-4-((tert-butoxycarbonyl)amino)-5-methoxy-5-oxopentanoic acid (1) (2.0 g, 7.662 mmol) DMF (20 mL) added EDC.HCl (2.19 g, 11.494 mmol), HOBT (1.55 g, 11.494 mmol), DIPEA (3.2 mL, 22.989 mmol) at 0° C. Then reaction mass was stirred for 15 min and then added N-methyl-2-phenylpropan-1-amine (2) (1.37 g, 9.195 mmol) at 0° C. and stirred at room temperature for 16 h. The progress of the reaction was monitored by TLC and LCMS. After 16 h, Reaction mixture was quenched with ice water (100 mL) and extracted with ethyl acetate (2×60 mL), combined organic layers were washed with brine solution (30 mL), dried over sodium sulfate and evaporated under reduced pressure. The crude residue was purified by normal phase chromatography with eluted 3% methanol in dichloromethane to afford methyl N2-(tert-butoxycarbonyl)-N5-(2-(ethylamino)-2-oxoethyl)-N5-methyl-L-glutaminate (3).

### Methyl N5-methyl-N5-(2-phenylpropyl)-L-glutaminate hydrochloride (4)

To a stirred solution of methyl N2-(tert-butoxycarbonyl)-N5-(2-(ethylamino)-2-oxoethyl)-N5-methyl-L-glutaminate 3 (1.6 g, 4.08 mmol) in 1,4-dioxane (11 mL) was added 4 M HCl in dioxane (11 mL) with drop wise at 0° C. and the reaction mixture was stirred at RT for 2 h. The progress of the reaction was monitored by TLC. After 2 h, the reaction mixture was evaporated to dryness under reduced pressure to obtain crude compound, the resulting crude triturated with diethyl ether to afford methyl N5-methyl-N5-(2-phenylpropyl)-L-glutaminate hydrochloride (4).

### Methyl N2-((S)-2-((((3-chlorobenzyl)oxy)carbonyl)amino)-3-cyclohexylpropanoyl)-N5-methyl-N5-(2-phenylpropyl)-L-glutaminate (5)

To a stirred solution of (S)-2-((((3-chlorobenzyl)oxy)carbonyl)amino)-3-cyclohexylpropanoic acid (acid fragment) (300 mg, 0.884 mmol) (synthesis reported in Compd-17 final report) in DMF (10 mL) was added EDC.HCl (253 mg, 1.327 mmol), HOBT (179 mg, 1.327 mmol), DIPEA (0.36 mL, 2.65 mmol) at 0° C. Then reaction mass was stirred for 15 min was added methyl N5-methyl-N5-(2-phenylpropyl)-L-glutaminate hydrochloride (4) (348 mg, 1.06 mmol) at 0° C. stirred at room temperature for 16 h. The progress of the reaction was monitored by TLC and LCMS. After 16 h, the reaction mixture was quenched with ice water (60 mL) and extracted with ethyl acetate (2×50 mL). Combined organic layers were washed with brine solution (50 mL), dried over sodium sulfate and evaporated under reduced pressure to get crude. Crude residue was purified by normal phase chromatography with eluted 5% methanol in dichloromethane to afford methyl N2-((S)-2-((((3-chlorobenzyl)oxy)carbonyl)amino)-3-cyclohexylpropanoyl)-N5-methyl-N5-(2-phenylpropyl)-L-glutaminate (5).

### 3-Chlorobenzyl ((2S)-3-cyclohexyl-1-(((2S)-1-hydroxy-5-(methyl(2-phenylpropyl)amino)-5-oxopentan-2-yl)amino)-1-oxopropan-2-yl)carbamate (6)

To a stirred solution of methyl N2-((S)-2-((((3-chlorobenzyl)oxy)carbonyl)amino)-3-cyclohexylpropanoyl)-N5-methyl-N5-(2-phenylpropyl)-L-glutaminate (5) (800 mg, 1.305 mmol) in DCM (10 mL) was added 2M LiBH₄ in THE (1.9 mL, 3.915 mmol) at 0° C. reaction mixture stirred for 3 h at 0° C. The progress of the reaction was monitored by TLC and LCMS. The reaction mixture was quenched with water (20 mL) and extracted with DCM (2×20 mL). The combined organic layer was washed with brine solution (20 mL) and the organic layer was dried over Na₂SO₄ and concentrated to get crude compound. Crude compound was purified by normal phase chromatography to afford 3-chlorobenzyl ((2S)-3-cyclohexyl-1-(((2S)-1-hydroxy-5-(methyl(2-phenylpropyl)amino)-5-oxopentan-2-yl)amino)-1-oxopropan-2-yl)carbamate (6).

### 3-Chlorobenzyl ((2S)-3-cyclohexyl-1-(((2S)-5-(methyl(2-phenylpropyl)amino)-1,5-dioxopentan-2-yl)amino)-1-oxopropan-2-yl)carbamate A49

To a stirred solution of 3-chlorobenzyl ((2S)-3-cyclohexyl-1-(((2S)-1-hydroxy-5-(methyl(2-phenylpropyl)amino)-5-oxopentan-2-yl)amino)-1-oxopropan-2-yl)carbamate (6) (250 mg, 0.426 mmol) in ethyl acetate (4 mL) was added Dess-Martin periodinane (542 mg, 1.279 mmol) at 0° C. and stirred at RT for 3 h. The progress of the reaction was monitored by TLC and LCMS. After 3 h, the reaction mixture was diluted with ethyl acetate (15 mL) and washed with sat. Hypo solution (3×20 mL) and sat. NaHCO₃ solution (3×20 mL). The organic layer was washed with brine solution (20 mL) and dried over Na₂SO₄ and concentrated to get crude compound. Crude was purified by prep HPLC to afford 3-chlorobenzyl ((2S)-3-cyclohexyl-1-(((2S)-5-(methyl(2-phenylpropyl)amino)-1,5-dioxopentan-2-yl)amino)-1-oxopropan-2-yl)carbamate A49

TLC system: 10% Methanol in DCM; Rf: 0.4

LCMS (ESI): m/z 584.58 (M+H)⁺

### 3-Chlorobenzyl ((2S)-3-cyclohexyl-1-(((2S)-1-(diethoxyphosphoryl)-1-hydroxy-5-(methyl(2-phenylpropyl)amino)-5-oxopentan-2-yl)amino)-1-oxopropan-2-yl)carbamate A102

To a stirred solution of 3-chlorobenzyl ((2S)-3-cyclohexyl-1-(((2S)-5-(methyl(2-phenylpropyl)amino)-1,5-dioxopentan-2-yl)amino)-1-oxopropan-2-yl)carbamate A49 (220 mg, 0.377 mmol) in DCM (3 mL) added DIPEA (0.17 mL, 1.13 mmol) followed by diethylphosphite (0.2 mL, 1.13 mmol) at 0° C. then reaction mixture stirred at RT for 16 h. The progress of the reaction was monitored by TLC and LCMS. After 16 h, the reaction mixture quenched with water (15 mL) and extracted with DCM (2×20 mL). The combined organic layer was washed with brine solution (10 mL) and dried over Na₂SO₄ and concentrated to get crude residue. It was purified by prep HPLC to afford 3-chlorobenzyl ((2S)-3-cyclohexyl-1-(((2S)-1-(diethoxyphosphoryl)-1-hydroxy-5-(methyl(2-phenylpropyl)amino)-5-oxopentan-2-yl)amino)-1-oxopropan-2-yl)carbamate A102.

TLC system: 10% Methanol in DCM; Rf: 0.4

LCMS (ESI): m/z 722.62 (M+H)⁺

### Methyl N2-((S)-2-((((3-chlorobenzyl)oxy)carbonyl)amino)-3-cyclohexylpropanoyl)-N5-methyl-N5-(2-phenylpropyl)-L-glutaminate (7)

To a stirred solution of (S)-4-((S)-2-((((3-chlorobenzyl)oxy)carbonyl)amino)-3-cyclohexylpropanamido)-5-methoxy-5-oxopentanoic acid (acid fragment-2) (1 g, 2.074 mmol) in DMF (20 mL) were added EDC.HCl (594 mg, 3.1119 mmol), HOBT (420 mg, 3.1119 mmol), DIPEA (1.5 mL, 6.224 mmol) and N-methyl-2-phenylpropan-1-amine (1) (370 mg, 2.489 mmol) at 0° C. and the reaction mixture was stirred at RT for 16 h. The progress of the reaction was monitored by TLC and LCMS. The reaction mixture was diluted with ice water (50 mL), extracted with ethyl acetate (2×50 mL). Combined organic layers were washed with brine solution (50 mL), dried over Na₂SO₄ and concentrated to get crude residue. The crude residue was purified by combi-flash compound eluted at 5% methanol in DCM to afford methyl N2-((S)-2-((((3-chlorobenzyl)oxy)carbonyl)amino)-3-cyclohexylpropanoyl)-N5-methyl-N5-(2-phenylpropyl)-L-glutaminate (7).

### N2-((S)-2-((((3-chlorobenzyl)oxy)carbonyl)amino)-3-cyclohexylpropanoyl)-N5-methyl-N5-(2-phenylpropyl)-L-glutamine (8)

To a stirred solution of methyl N2-((S)-2-((((3-chlorobenzyl)oxy)carbonyl)amino)-3-cyclohexylpropanoyl)-N5-methyl-N5-(2-phenylpropyl)-L-glutaminate (7) (600 mg, 0.978 mmol) in THE (4 mL), MeOH (4 mL) and water (1 mL), was added lithium hydroxide (46 mg, 1.957 mmol) at room temperature and stirred at same for 3 h. The progress of the reaction was monitored by TLC and LCMS. After 3 h, the reaction mixture completely distilled under reduced pressure, crude compound acidified with 1N HCL solution up to pH ˜4, and extracted with ethyl acetate (2×10 mL). Combined organic layers were washed with brine solution (20 mL), dried over Na₂SO₄ and concentrated to get N2-((S)-2-((((3-chlorobenzyl)oxy)carbonyl)amino)-3-cyclohexylpropanoyl)-N5-methyl-N5-(2-phenylpropyl)-L-glutamine (8).

### 3-chlorobenzyl ((2S)-3-cyclohexyl-1-(((2S)-1-(methoxy(methyl)amino)-5-(methyl(2-phenylpropyl)amino)-1,5-dioxopentan-2-yl)amino)-1-oxopropan-2-yl)carbamate A35

To a stirred solution of get N2-((S)-2-((((3-chlorobenzyl)oxy)carbonyl)amino)-3-cyclohexylpropanoyl)-N5-methyl-N5-(2-phenylpropyl)-L-glutamine (8) (500 mg, 0.834 mmol) in THE (5 mL) and DCM (5 mL), added EDC.HCl (191 mg, 1.001 mmol), HOBT (135 mg, 1.001 mmol), N-methylmorpholine (0.1 mL, 1.001 mmol) and N,O-dimethylhydroxylamine (97 mg, 0.001 mmol) at 0° C. simultaneously and stirred at room temperature for 16 h. The progress of the reaction was monitored by TLC and LCMS. Then the reaction mixture was diluted with ice water (15 mL), extracted with ethyl acetate (2×10 mL). Combined organic layers were washed with brine solution (20 mL), dried over Na₂SO₄ and concentrated to get crude residue. Crude was purified by prep-HPLC purification afforded 3-chlorobenzyl ((2S)-3-cyclohexyl-1-(((2S)-1-(methoxy(methyl)amino)-5-(methyl(2-phenylpropyl)amino)-1,5-dioxopentan-2-yl)amino)-1-oxopropan-2-yl)carbamate A35.

TLC system: 10% Methanol in DCM; Rf: 0.6

LCMS (ESI): m/z 643.56 (M+H)

### Synthesis of Compounds A36, A15, A30, and A26

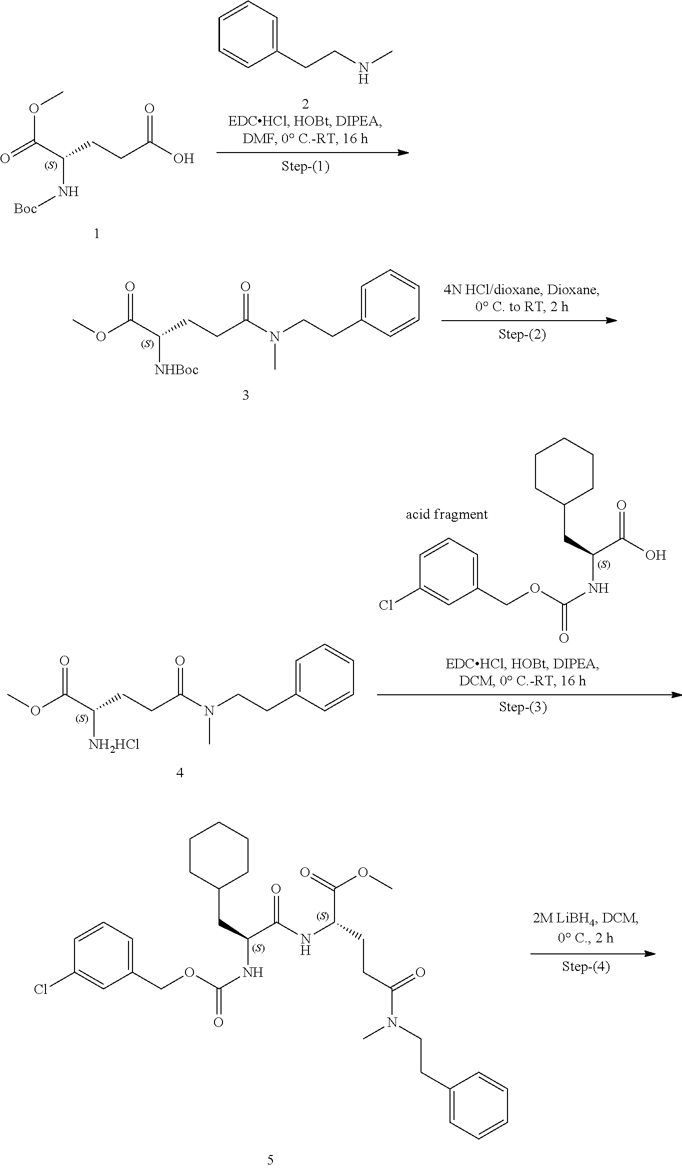

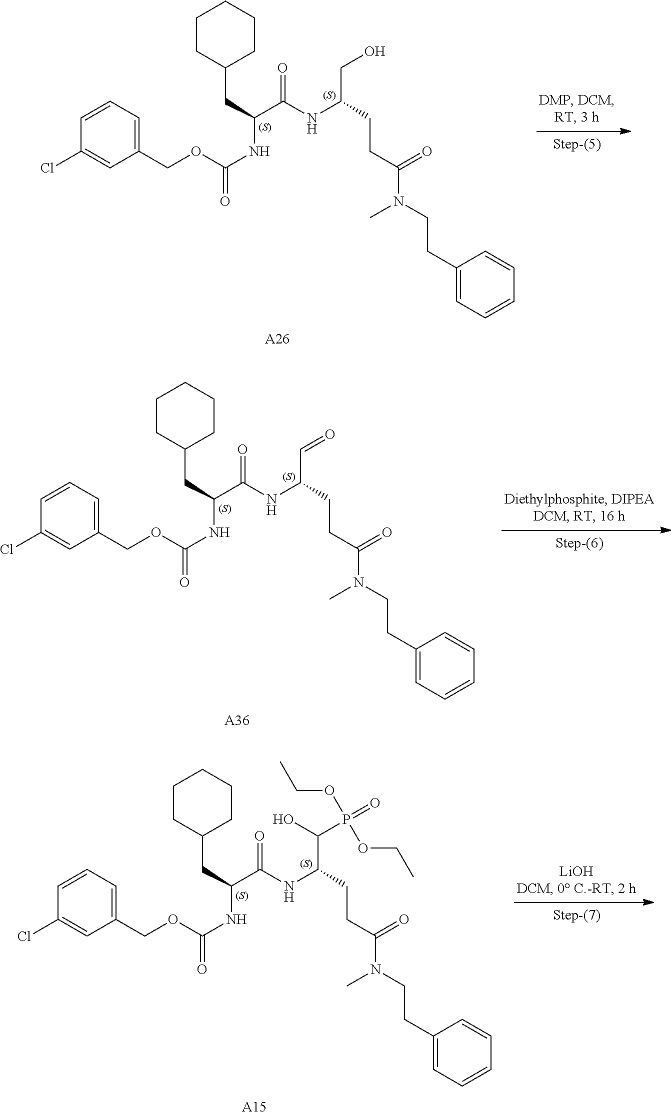

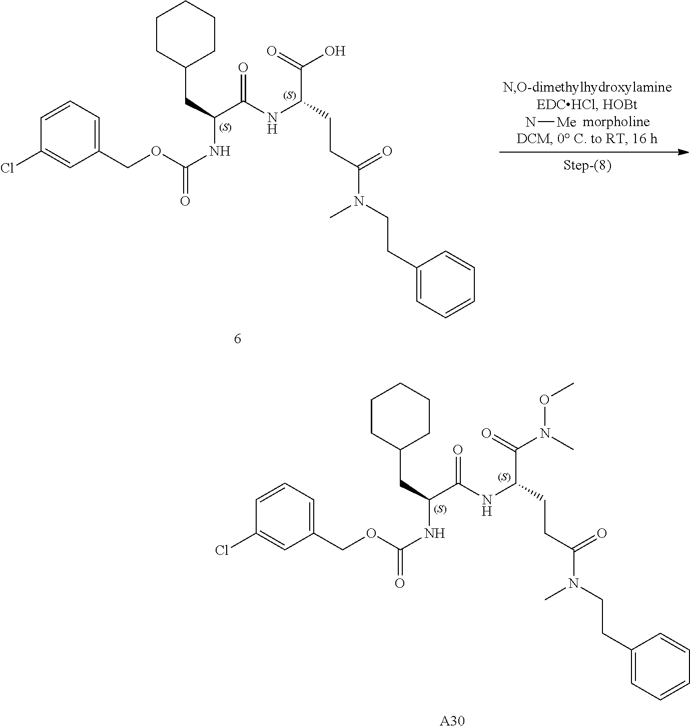

### Methyl N2-(tert-butoxycarbonyl)-N5-methyl-N5-phenethyl-L-glutaminate (2)

To a stirred solution of (S)-4-((tert-butoxycarbonyl)amino)-5-methoxy-5-oxopentanoic acid (1) (2×1 g, 0.3.831 mmol) in DMF (10 mL) was added HOBt (775 mg, 5.74 mmol), EDC.HCl (1.1 g, 5.74 mmol) and DIPEA (2.11 mL, 11.49 mmol) followed by N-methyl-2-phenylethan-1-amine (2) (569 mg, 4.214 mmol) at 0° C. Reaction mixture was stirred at room temperature for 16 h. The progress of the reaction was monitored by TLC. After consumption of starting material, the reaction mixture was quenched with water (10 mL) and extracted with EtOAc (2×100 mL). Combined organic layer was washed with brine solution (10 mL), dried over Na₂SO₄ and concentrated to get crude residue; Crude compound was purified by normal phase chromatography to afford methyl N²-(tert-butoxycarbonyl)-N⁵-methyl-N⁵-phenethyl-L-glutaminate (3).

### Methyl N⁵-methyl-N⁵-phenethyl-L-glutaminate hydrochloride (4)

To a stirred solution of methyl N2-(tert-butoxycarbonyl)-N5,N5-dimethyl-L-glutaminate (3) (2 g, 5.291 mmol) in 1,4-dioxane (10 mL) was added 4 M HCl in dioxane (10 mL) with drop wise at 0° C. Reaction mixture was stirred at RT for 2 h. The progress of the reaction was monitored by TLC. After consumption of starting material, the reaction mixture was evaporated under reduced pressure to obtain crude compound, the resulting crude triturated with diethyl ether to afford methyl N5-methyl-N5-phenethyl-L-glutaminate hydrochloride (4).

### Methyl N2-((S)-2-((((3-chlorobenzyl) oxy) carbonyl) amino)-3-cyclohexylpropanoyl)-N5, N5-dimethyl-L-glutaminate (5)

To a stirred solution of (S)-2-((((3-chlorobenzyl)oxy)carbonyl)amino)-3-cyclohexylpropanoic acid (acid fragment) (1 g, 2.942 mmol) (synthesis reported in Compd-17 final report) in DMF (10 mL) was added HOBt (597 mg, 4.42 mmol), EDC.HCl (844 mg, 4.42 mmol) and DIPEA (1.6 mL, 8.849 mmol) followed by methyl N5-methyl-N5-phenethyl-L-glutaminate hydrochloride (4) (1.1 g, 3.539 mmol) at 0° C. Reaction mixture was stirred at room temperature for 16 h. The progress of the reaction was monitored by TLC. After consumption of starting material, the reaction mixture was quenched with water (10 mL) and extracted with EtOAc (2×100 mL). Combined organic layer was washed with brine solution (10 mL), dried over Na₂SO₄ and concentrated to get crude; Crude compound was purified by normal phase chromatography to afford pure compound methyl N2-((S)-2-(((3-chlorobenzyl) oxy) carbonyl) amino)-3-cyclohexylpropanoyl)-N5, N5-dimethyl-L-glutaminate (5).

### 3-chlorobenzyl ((S)-3-cyclohexyl-1-(((S)-1-hydroxy-5-(methyl(phenethyl)amino)-5-oxopentan-2-yl)amino)-1-oxopropan-2-yl)carbamate A26

To a stirred solution of Methyl N2-((S)-2-(((3-chlorobenzyl) oxy) carbonyl) amino)-3-cyclohexylpropanoyl)-N5, N5-dimethyl-L-glutaminate (5) (600 mg, 1.00 mmol) in DCM (5 mL) was added 2M LiBH₄ in THE (1.5 mL, 3 mmol) at 0° C., reaction mixture stirred for 2 h at 0° C. Reaction mixture was quenched with water (20 mL) and extracted with DCM (2×20 mL). Organic layer was washed with brine solution, dried over Na₂SO₄ and concentrated to get crude. Crude compound was purified by normal phase chromatography to afford 3-chlorobenzyl ((S)-3-cyclohexyl-1-(((S)-1-hydroxy-5-(methyl(phenethyl)amino)-5-oxopentan-2-yl)amino)-1-oxopropan-2-yl)carbamate A26.

TLC system: 10% MeOH in DCM; Rf: 0.2

LCMS (ESI): m/z 572.14 (M+H)⁺

### 3-chlorobenzyl ((S)-3-cyclohexyl-1-(((S)-5-(methyl(phenethyl)amino)-1,5-dioxopentan-2-yl)amino)-1-oxopropan-2-yl)carbamate A36

To a stirred solution of 3-chlorobenzyl((S)-3-cyclohexyl-1-(((S)-1-hydroxy-5-(methyl(phenethyl) amino)-5-oxopentan-2-yl)amino)-1-oxopropan-2-yl)carbamate (A26) (400 mg, 0.705 mmol) was dissolved in DCM (5 mL) was added Dess-Martin periodinane (891 mg, 2.101 mmol) at 0° C. and stirred at RT for 3 h. Reaction mixture was diluted with DCM (10 mL) and washed with sat. Hypo solution (3×10 mL), sat. NaHCO₃ solution (3×20 mL). Organic layer was dried over anhydrous Na₂SO₄, filtered and concentrated to get crude compound. The crude compound was triturated with diethyl ether to afford 3-chlorobenzyl ((S)-3-cyclohexyl-1-(((S)-5-(methyl (phenethyl) amino)-1,5-dioxopentan-2-yl)amino)-1-oxopropan-2-yl)carbamate A36.

TLC system: 10% MeOH in DCM; Rf: 0.4

LCMS (ESI): m/z 570.57 (M+H)⁺

### 3-Chlorobenzyl ((2S)-3-cyclohexyl-1-(((2S)-1-(diethoxyphosphoryl)-1-hydroxy-5-(methyl(phenethyl)amino)-5-oxopentan-2-yl)amino)-1-oxopropan-2-yl)carbamate A15

To a stirred solution of 3-chlorobenzyl ((S)-3-cyclohexyl-1-(((S)-5-(methyl (phenethyl) amino)-1,5-dioxopentan-2-yl)amino)-1-oxopropan-2-yl)carbamate A36 (450 mg, 0.7908 mmol) was dissolved in DCM (10 mL) was added DIPEA (0.437 mL, 2.372 mmol) and diethyl phosphite (327 mg, 2.372 mmol) at 0° C. and stirred at RT for 16 h. React ion mixture was quenched with ice water (10 mL) extracted with DCM (3×20 mL). The combined organic layer was dried over anhydrous Na₂SO₄, filtered and concentrated to get crude compound. It was washed with n-pentane (2×20 mL) and purified by prep HPLC to afforded pure 3-chlorobenzyl ((2S)-3-cyclohexyl-1-(((2S)-1-(diethoxyphosphoryl)-1-hydroxy 5 (methyl (phenethyl) amino)-5-oxopentan-2-yl) amino)-1-oxopropan-2-yl)carbamate A15.

TLC system: 100% EtOAc; Rf: 0.3

LCMS (ESI): m/z 708.3 (M+H)⁺

### N2-((S)-2-((((3-chlorobenzyl)oxy)carbonyl)amino)-3-cyclohexylpropanoyl)-N5-methyl-N5-phenethyl-L-glutamine(6)

To a stirred solution of methyl methyl N2-((S)-2-((((3-chlorobenzyl)oxy)carbonyl)amino)-3-cyclohexylpropanoyl)-N5-methyl-N5-phenethyl-L-glutaminate (5) (500 mg, 0.834 mmol) in THE (5 mL) and water (2.5 mL), was added lithium hydroxide (60 mg, 2.504 mmol) at 0° C. and stirred at room temperature for 3 h. The progress of the reaction was monitored by TLC and LCMS. After 3 h, the reaction mixture completely distilled under reduced pressure, crude compound acidified with 2N HCL solution up to pH ˜4, and extracted with ethyl acetate (2×10 mL), dried over sodium sulfate, concentrated under reduced pressure to afford crude compound N2-((S)-2-((((3-chlorobenzyl)oxy)carbonyl)amino)-3-cyclohexylpropanoyl)-N5-methyl-N5-phenethyl-L-glutamine (6).

### 3-chlorobenzyl ((S)-3-cyclohexyl-1-(((S)-1-(methoxy(methyl)amino)-5-(methyl(phenethyl)amino)-1,5-dioxopentan-2-yl)amino)-1-oxopropan-2-yl)carbamate A30

To a stirred solution of N2-((S)-2-((((3-chlorobenzyl)oxy)carbonyl)amino)-3-cyclohexylpropanoyl)-N5-methyl-N5-phenethyl-L-glutamine (6) (500 mg, 0.8547 mmol) in THE (5 mL) and DCM (5 mL), added EDC.HCl (225 mg, 1.025 mmol), HOBT (159 mg, 1.025 mmol), N-methylmorpholine (0.129 mL, 1.025 mmol) and N,O-dimethylhydroxylamine (114 mg, 1.025 mmol) at 0° C. simultaneously and stirred at room temperature for 16 h. The progress of the reaction was monitored by TLC and LCMS. After 16 h, the reaction mixture was diluted with ice water (10 mL), extracted with ethyl acetate (2×10 mL). The combined organic layer was dried over sodium sulfate, filtered and evaporated under reduced pressure. The crude residue was purified by combi-flash, compound eluted at 70% ethyl acetate in pet ether followed by second purification using Prep-HPLC to afforded 3-chlorobenzyl ((S)-3-cyclohexyl-1-(((S)-1-(methoxy(methyl)amino)-5-(methyl (phenethyl)amino)-1,5-dioxopentan-2-yl)amino)-1-oxopropan-2-yl)carbamate A30.

TLC system: 10% Methanol in DCM; Rf: 0.5

LCMS (ESI): m/z 623.53 (M+H)⁺

### Synthesis of Compounds A66, A104, and A37

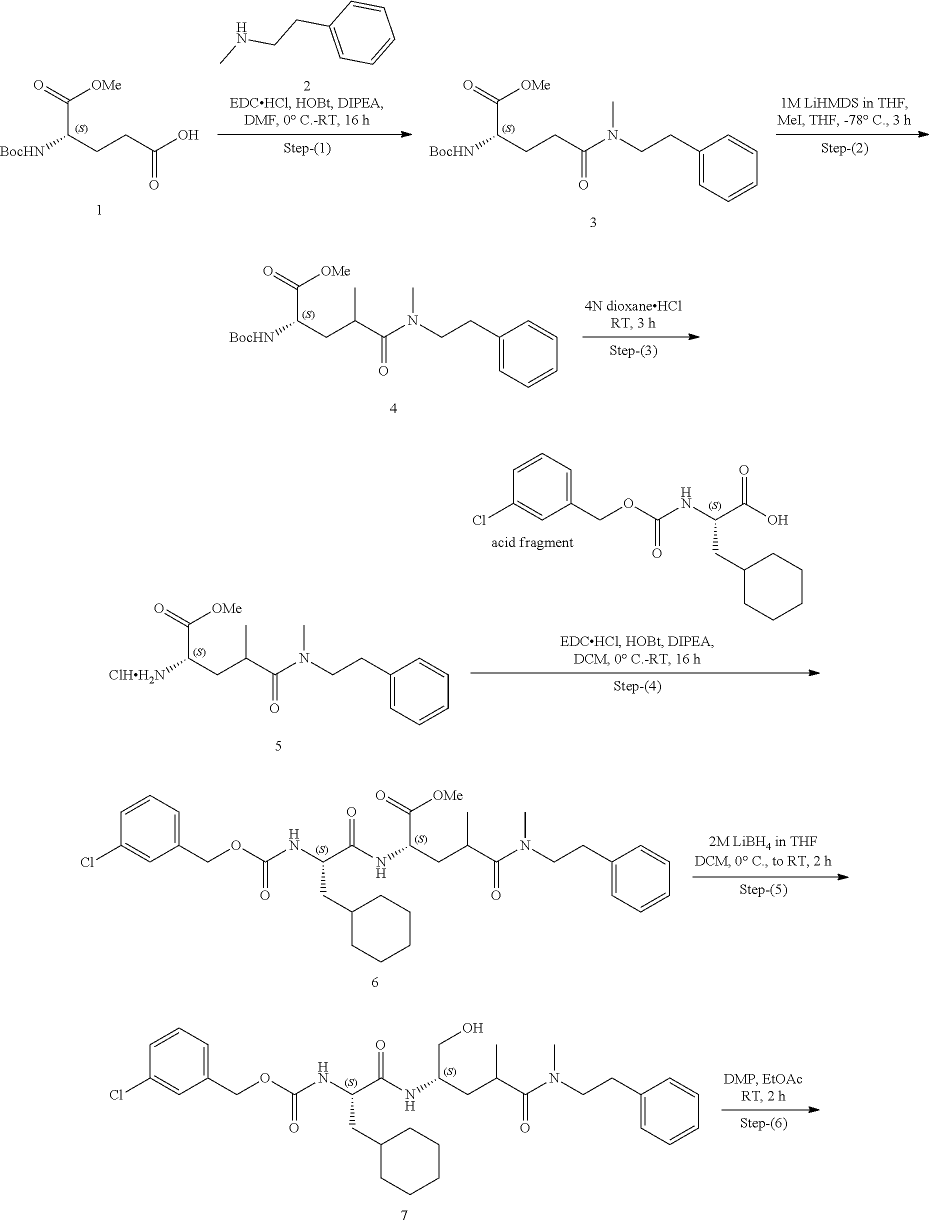

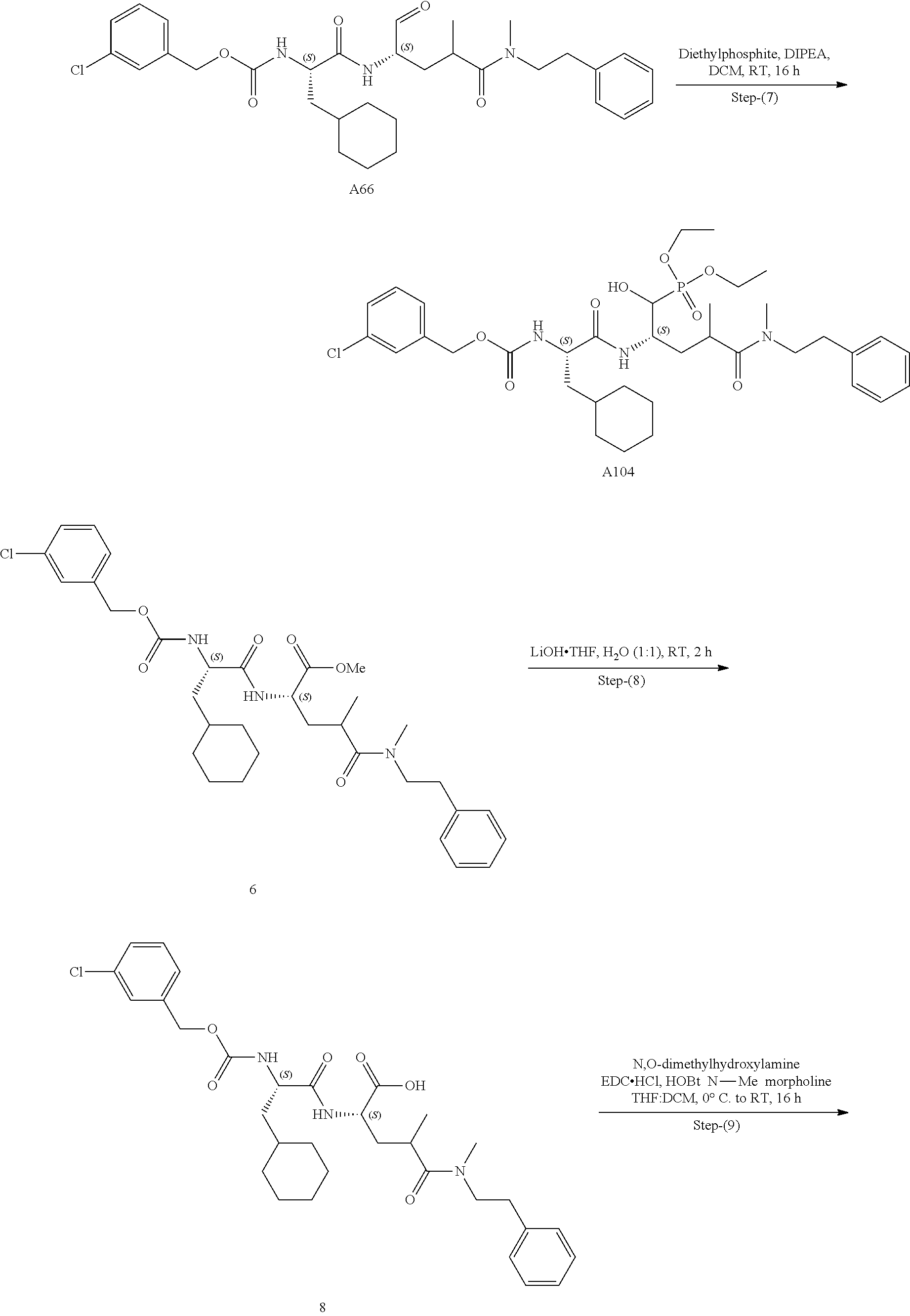

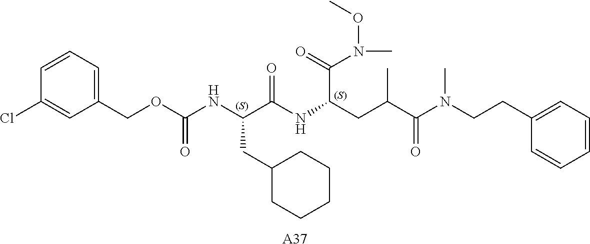

### Methyl N2-(tert-butoxycarbonyl)-N5-methyl-N5-phenethyl-L-glutaminate (3)

To a stirred solution of (S)-4-((tert-butoxycarbonyl)amino)-5-methoxy-5-oxopentanoic acid (1) (500 mg, 1.92 mmol) in DMF (10 mL) was added EDC.HCl (548 mg, 2.87 mmol), HOBT (387 mg, 2.87 mmol), DIPEA (0.9 mL, 5.73 mmol) and N-methyl-2-phenylethan-1-amine (2) (310 mg, 2.29 mmol) at 0° C. simultaneously and stirred at room temperature for 16 h. The progress of the reaction was monitored by TLC and LCMS. Reaction mixture was diluted with ice water (30 mL), extracted with ethyl acetate (2×30 mL), organic layer was dried over sodium sulfate and evaporated under reduced pressure. The crude residue was purified by silica gel column by eluting with 40% ethyl acetate in pet ether to afford methyl N2-(tert-butoxycarbonyl)-N5-methyl-N5-phenethyl-L-glutaminate (3).

### Methyl (2S)-2-((tert-butoxycarbonyl) amino)-4-methyl-5-(methyl(phenethyl)amino)-5-oxopentanoate (4)

To a stirred solution of methyl N2-(tert-butoxycarbonyl)-N5-methyl-N5-phenethyl-L-glutaminate (3) (1.5 g, 3.96 mmol) in THE (30 mL) added 1M LiHMDS (9.9 mL, 9.92 mmol) at −78° C. and stirred for 1 h then added methyl iodide (1 mL, 15.87 mmol) in THE and stirred at −78° C. for 3 h. The progress of the reaction was monitored by TLC and LCMS. After 3 h, the reaction mixture was quenched with sat. Ammonium chloride solution and extracted with ethyl acetate (2×50 mL), the combined organic layer was dried over sodium sulfate and evaporated under reduced pressure. The crude residue was purified by silica gel column by eluting with 50% ethyl acetate in pet ether to afford methyl (2S)-2-((tert-butoxycarbonyl)amino)-4-methyl-5-(methyl(phenethyl)amino)-5-oxopentanoate (4).

### Methyl (2S)-2-amino-4-methyl-5-(methyl (phenethyl) amino)-5-oxopentanoate hydrochloride (5)

To a stirred solution of methyl (2S)-2-((tert-butoxycarbonyl) amino)-4-methyl-5-(methyl (phenethyl) amino)-5-oxopentanoate (4) (1.5 g, 3.82 mmol) in 1, 4-dioxane (15 mL) was added 4N HCl in dioxane (15 mL) with drop wise at 0° C. and the reaction mixture was stirred at RT for 3 h. The progress of the reaction was monitored by TLC. After consumption of starting material, the reaction mixture was evaporated under reduced pressure to obtained crude compound, the resulting crude was triturated with diethyl ether to afford methyl (2S)-2-amino-4-methyl-5-(methyl(phenethyl)amino)-5-oxopentanoate hydrochloride (5).

### Methyl (2S)-2-((S)-2-((((3-chlorobenzyl) oxy) carbonyl) amino)-3-cyclohexylpropanamido)-4-methyl-5-(methyl (phenethyl) amino)-5-oxopentanoate (6)

To a stirred solution of (S)-2-((((3-chlorobenzyl)oxy)carbonyl)amino)-3-cyclohexylpropanoic acid (1.2 g, 3.53 mmol) DMF (15 mL) added EDC.HCl (1.01 g, 5.30 mmol), HOBT (0.71 g, 5.30 mmol), DIPEA (0.9 mL, 5.30 mmol) and methyl (2S)-2-amino-4-methyl-5-(methyl(phenethyl)amino)-5-oxopentanoate hydrochloride (5) (1.39 g, 4.24 mmol) at 0° C. simultaneously and stirred at room temperature for 16 h. The progress of the reaction was monitored by TLC and LCMS. Reaction mixture was diluted with ice water (30 mL), extracted with ethyl acetate (2×40 mL), the combined organic layer was dried over sodium sulfate and evaporated under reduced pressure. The crude residue was purified by grace NP, compound eluted at 70% Ethyl acetate in pet ether to afford methyl (2S)-2-((S)-2-((((3-chlorobenzyl) oxy) carbonyl) amino)-3-cyclohexylpropanamido)-4-methyl-5-(methyl (phenethyl) amino)-5-oxopentanoate (6).

### 3-Chlorobenzyl ((2S)-3-cyclohexyl-1-(((2S)-1-hydroxy-4-methyl-5-(methyl (phenethyl) amino)-5-oxopentan-2-yl) amino)-1-oxopropan-2-yl) carbamate (7)

At 0° C., to a stirred solution of methyl (2S)-2-((S)-2-((((3-chlorobenzyl) oxy) carbonyl) amino)-3-cyclohexylpropanamido)-4-methyl-5-(methyl (phenethyl) amino)-5-oxopentanoate (6) (300 mg, 0.489 mmol) in DCM (10 mL) was added 2M LiBH₄ in THE (0.5 mL, 0.978 mmol) and the reaction mixture was stirred at same temperature for 2 h. The progress of the reaction was monitored by TLC and LCMS. Reaction mixture was quenched with water (10 mL) and extracted with DCM (2×20 mL). Organic layer was washed with brine solution (20 mL), dried over Na₂SO₄ and concentrated to afford 3-chlorobenzyl ((2S)-3-cyclohexyl-1-(((2S)-1-hydroxy-4-methyl-5-(methyl (phenethyl) amino)-5-oxopentan-2-yl) amino)-1-oxopropan-2-yl) carbamate (7).

### 3-Chlorobenzyl ((2S)-3-cyclohexyl-1-(((2S)-4-methyl-5-(methyl (phenethyl) amino)-1, 5-dioxopentan-2-yl) amino)-1-oxopropan-2-yl)carbamate A66

To a stirred solution of 3-chlorobenzyl ((2S)-3-cyclohexyl-1-(((2S)-1-hydroxy-4-methyl-5-(methyl (phenethyl) amino)-5-oxopentan-2-yl) amino)-1-oxopropan-2-yl) carbamate (7) (190 mg, 0.324 mmol) was dissolved in ethyl acetate (5 mL) was added Dess-Martin periodinane (412 mg, 0.972 mmol) at 0° C. and stirred at RT for 2 h. The progress of the reaction was monitored by TLC and LCMS. Reaction mixture was filtered through celite bed and filtrate was washed with sat. NaHCO₃ solution (3×10 mL) followed by sat. Hypo solution (3×10 mL). Organic layer was dried over anhydrous Na₂SO₄, filtered and concentrated to get crude compound. The crude compound was purified by prep HPLC to afford 3-chlorobenzyl ((2S)-3-cyclohexyl-1-(((2S)-4-methyl-5-(methyl (phenethyl) amino)-1, 5-dioxopentan-2-yl) amino)-1-oxopropan-2-yl) carbamate A66.

TLC system: 70% Ethyl acetate in Pet ether; Rf: 0.5

LCMS (ESI): m/z 584.55 (M+H)⁺

### 3-Chlorobenzyl ((2S)-3-cyclohexyl-1-(((2S)-1-(diethoxyphosphoryl)-1-hydroxy-4-methyl-5-(methyl (phenethyl)amino)-5-oxopentan-2-yl)amino)-1-oxopropan-2-yl)carbamate A104

To a stirred solution of 3-chlorobenzyl ((2S)-3-cyclohexyl-1-(((2S)-4-methyl-5-(methyl (phenethyl) amino)-1, 5-dioxopentan-2-yl) amino)-1-oxopropan-2-yl) carbamate A66 (300 mg crude, 0.514 mmol) in DCM (10 mL) added DIPEA (0.25 mL, 1.543 mmol) followed by addition of diethylphosphite (0.2 mL, 1.543 mmol) and the reaction mixture stirred at RT for 16 h. The progress of the reaction was monitored by TLC and LCMS. After 16 h, the reaction mixture quenched with ammonium chloride (15 mL) and extracted with DCM (2×20 mL). Combined organic layer was dried over anhydrous Na₂SO₄, and evaporated to afford crude residue. It was purified prep HPLC to afford 3-chlorobenzyl ((2S)-3-cyclohexyl-1-(((2S)-1-(diethoxyphosphoryl)-1-hydroxy-4-methyl-5-(methyl (phenethyl) amino)-5-oxopentan-2-yl) amino)-1-oxopropan-2-yl) carbamate A104.

TLC system: 5% Methanol in dichloromethane; Rf: 0.4

LCMS (ESI): m/z 722.67 (M+H)⁺

### (2S)-2-((S)-2-((((3-chlorobenzyl) oxy) carbonyl) amino)-3-cyclohexylpropanamido)-4-methyl-5-(methyl (phenethyl) amino)-5-oxopentanoic acid (8)

To a stirred solution of methyl (2S)-2-((S)-2-((((3-chlorobenzyl)oxy)carbonyl)amino)-3-cyclohexylpropanamido)-4-methyl-5-(methyl(phenethyl)amino)-5-oxopentanoate (6) (300 mg, 0.489 mmol) in THE (5 mL) and water (5 mL) was added lithium hydroxide (23 mg, 0.978 mmol) at 0° C. and stirred at room temperature for 2 h. The progress of the reaction was monitored by TLC and LCMS. Reaction mixture completely distilled under reduced pressure, crude compound was acidified with 2N HCl solution up to pH ˜3 and extracted with ethyl acetate (2×20 mL), dried over sodium sulfate, concentrated under reduced pressure to afford (2S)-2-((S)-2-((((3-chlorobenzyl)oxy)carbonyl)amino)-3-cyclohexylpropanamido)-4-methyl-5-(methyl(phenethyl)amino)-5-oxopentanoic acid (8).

### 3-Chlorobenzyl ((2S)-3-cyclohexyl-1-(((2S)-1-(methoxy (methyl) amino)-4-methyl-5-(methyl (phenethyl) amino)-1, 5-dioxopentan-2-yl) amino)-1-oxopropan-2-yl) carbamate A37

At 0° C., to a stirred solution of (2S)-2-((S)-2-((((3-chlorobenzyl)oxy)carbonyl)amino)-3-cyclohexylpropanamido)-4-methyl-5-(methyl(phenethyl)amino)-5-oxopentanoic acid (8) (220 mg, 0.367 mmol) in THE (5 mL) and DCM (5 mL) was added EDC.HCl (105 mg, 0.55 mmol), HOBT (74 mg, 0.55 mmol), N-methylmorpholine (0.12 mL, 1.101 mmol) and N,O-dimethylhydroxylamine (42 mg, 0.44 mmol) simultaneously and stirred at room temperature for 16 h. The progress of the reaction was monitored by TLC and LCMS. Then the reaction mixture was quenched with ice water (20 mL) and extracted with ethyl acetate (2×20 mL). The combined organic layer was dried over sodium sulfate, filtered and evaporated under reduced pressure. The crude residue was purified by prep-HPLC to afforded 3-chlorobenzyl ((2S)-3-cyclohexyl-1-(((2S)-1-(methoxy(methyl) amino)-4-methyl-5-(methyl (phenethyl) amino)-1,5-dioxopentan-2-yl)amino)-1-oxopropan-2-yl)carbamate (A37.

TLC system: 5% Methanol in DCM; Rf: 0.4

LCMS (ESI): m/z 643.55 (M+H)⁺

### Synthesis of Compounds A38, A22, and A29

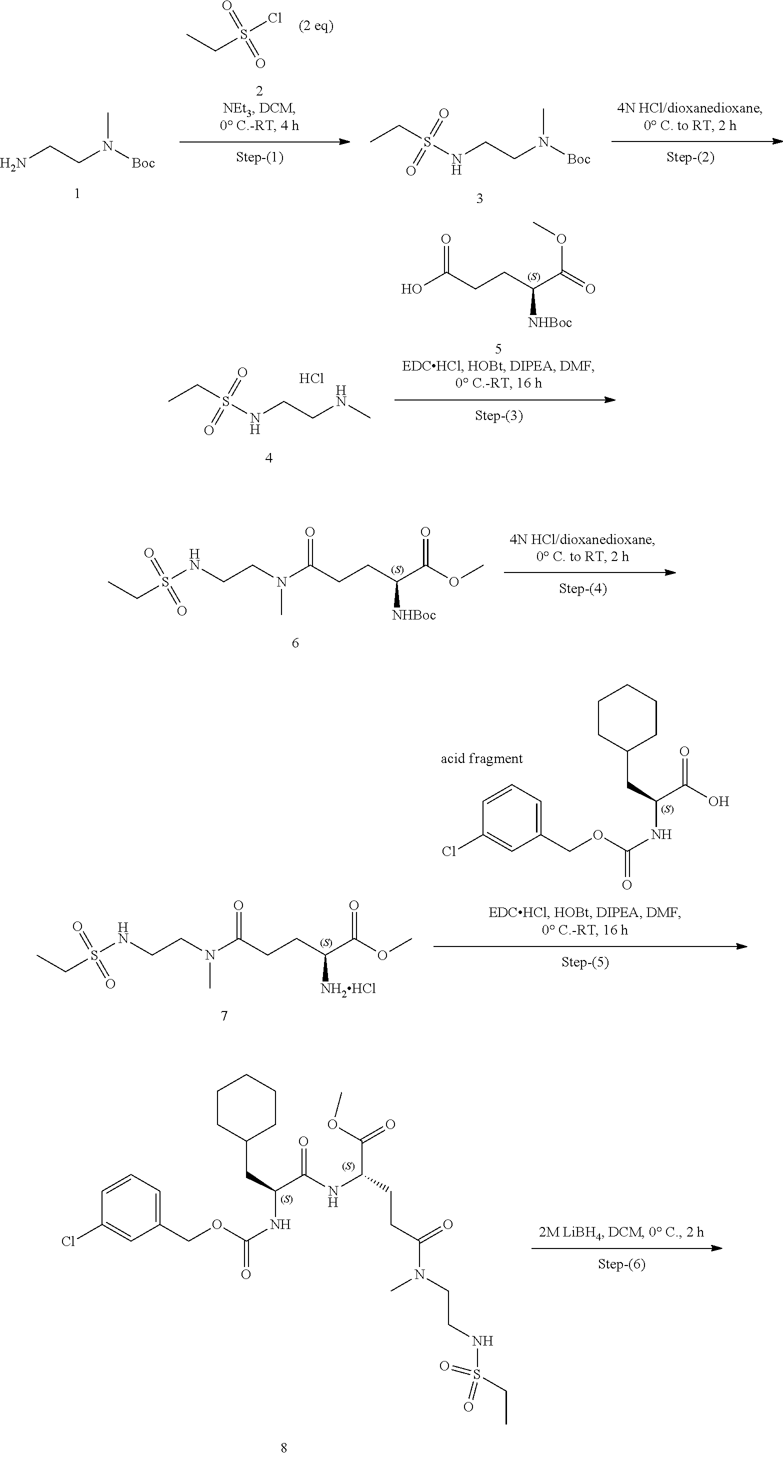

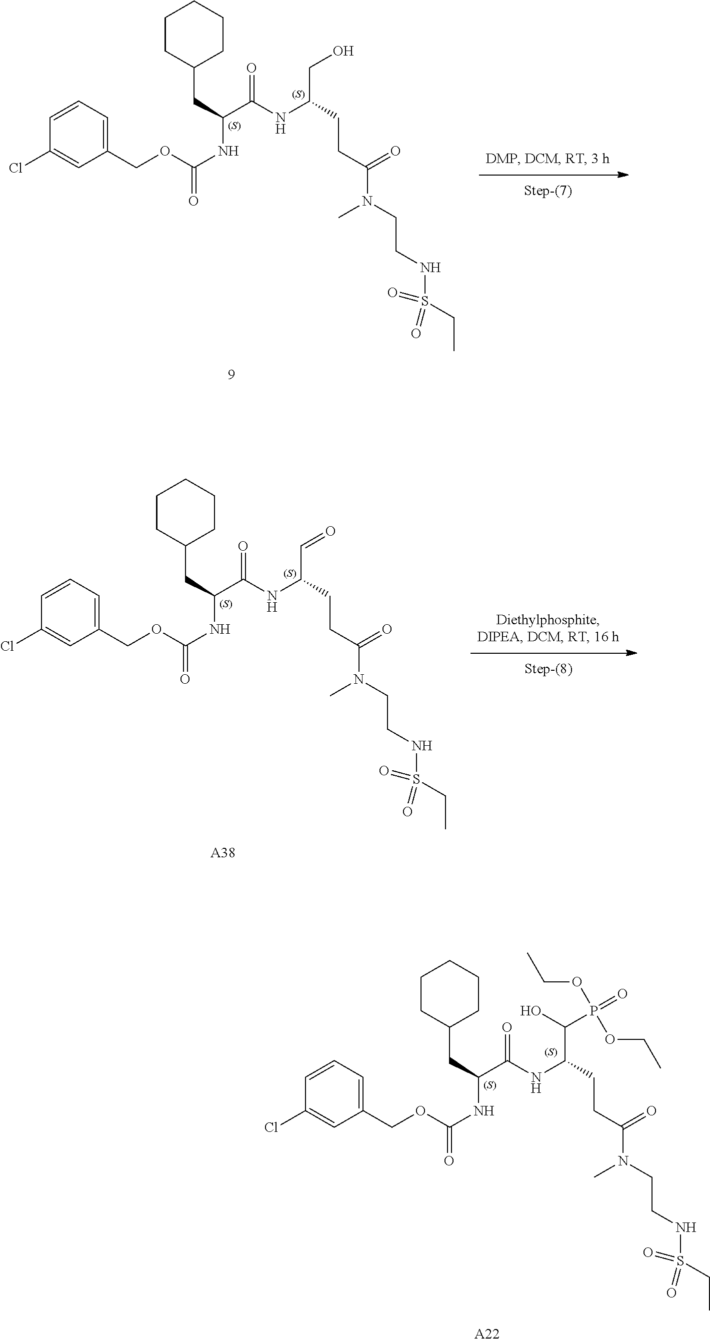

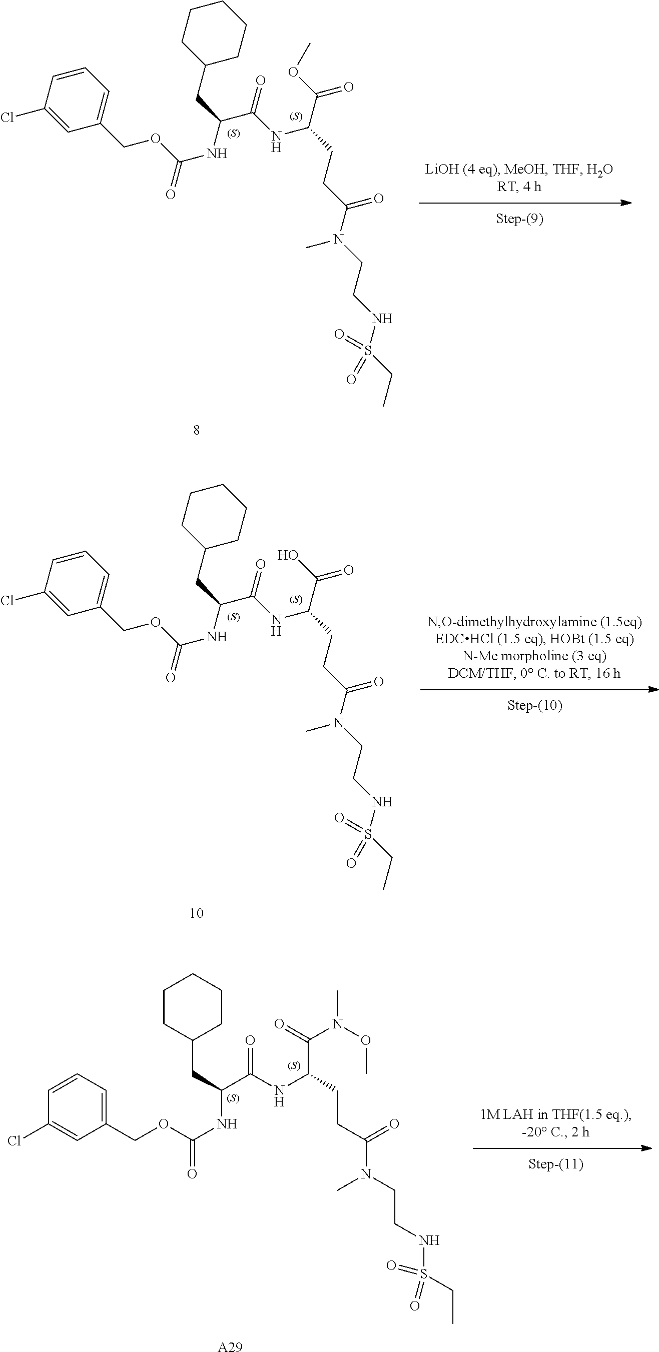

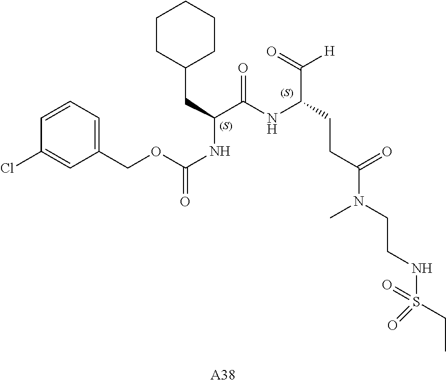

### Tert-butyl (2-(ethylsulfonamido)ethyl)(methyl)carbamate (3)

To a stirred solution of tert-butyl (2-aminoethyl)(methyl)carbamate (1) (4 g, 22.98 mmol) in DCM (40 mL) at 0° C. was added triethylamine (9.3 mL, 68.94 mmol) followed by ethanesulfonyl chloride (2) (3.5 g, 27.58 mmol) and the reaction mixture was stirred at RT for 16 h. The progress of the reaction was monitored by TLC. After consumption of starting material, the reaction mixture was quenched with ice water (50 mL) and extracted with DCM (2×50 mL). Combined organic layer was washed with brine solution (50 mL), dried over Na₂SO₄ and concentrated to afford tert-butyl (2-(ethylsulfonamido) ethyl) (methyl) carbamate (3).

### N-(2-(Methylamino)ethyl)ethanesulfonamide hydrochloride (4)

To a stirred solution of tert-butyl (2-(ethylsulfonamido) ethyl) (methyl) carbamate (3) (3.5 g, 13.15 mmol) in 1,4-dioxane (20 mL) at 0° C. was added 4N HCl in dioxane (20 mL) drop wise and the resulting reaction mixture was stirred for 3 h at RT. After consumption of starting material, the volatiles were removed under vacuo. Crude residue was triturated with diethylether (2×20 mL) to afford N-(2-(methylamino)ethyl)ethanesulfonamide hydrochloride (4).

### Methyl N²-(tert-butoxycarbonyl)-N⁵-(2-(ethylsulfonamido)ethyl)-N⁵-methyl-L-glutaminate (6)

To a stirred solution of (S)-4-((tert-butoxycarbonyl)amino)-5-methoxy-5-oxopentanoic acid (5) (2 g, 7.6628 mmol) in DMF (20 mL) at 0° C. was added HOBt (1.55 g, 11.494 mmol), EDC.HCl (2.19 g, 11.494 mmol) and DIPEA (4.16 mL, 22.988 mmol) followed by N-(2-(methylamino)ethyl)ethanesulfonamide (4) (1.85 g, 9.195 mmol) and the reaction mixture was stirred at room temperature for 16 h. The progress of the reaction was monitored by TLC and LCMS. After consumption of starting material, the reaction mixture was quenched with ice water (50 mL) and extracted with EtOAc (2×100 mL). Combined organic layers were washed with brine solution (50 mL), dried over Na₂SO₄ and concentrated to get crude residue. It was purified by normal phase silica chromatography to afford methyl N²-(tert-butoxycarbonyl)-N⁵-(2-(ethylsulfonamido)ethyl)-N⁵-methyl-L-glutaminate (6).

### Methyl N⁵-(2-(ethylsulfonamido)ethyl)-N⁵-methyl-L-glutaminate hydrochloride (7)

To a stirred solution of methyl N²-(tert-butoxycarbonyl)-N⁵-(2-(ethylsulfonamido)ethyl)-N⁵-methyl-L-glutaminate (6) (1.9 g, 4.645 mmol) in 1,4-dioxane (20 mL) at 0° C. was added 4N HCl in dioxane (20 mL) drop wise and the resulting reaction mixture was stirred for 2 h at RT. After consumption of starting material, the volatiles were removed under vacuo. Crude residue was triturated with diethylether twice (2×30 mL) to afford methyl N⁵-(2-(ethylsulfonamido)ethyl)-N⁵-methyl-L-glutaminate hydrochloride (7)

### Methyl N²—((S)-2-((((3-chlorobenzyl)oxy)carbonyl)amino)-3-cyclohexylpropanoyl)-N⁵-(2-(ethylsulfonamido)ethyl)-N⁵-methyl-L-glutaminate (8)

To a stirred solution of (S)-2-((((3-chlorobenzyl)oxy)carbonyl)amino)-3-cyclohexylpropanoic acid (acid fragment) (1.5 g, 4.427 mmol) in DMF (15 mL) at 0° C. was added HOBt (0.9 g, 6.636 mmol), EDC.HCl (1.26 g, 6.636 mmol) and DIPEA (2.3 mL, 13.272 mmol) followed by methyl N⁵-(2-(ethylsulfonamido)ethyl)-N⁵-methyl-L-glutaminate hydrochloride (7) (1.8 g, 5.309 mmol). Reaction mixture was stirred at room temperature for 16 h. The progress of the reaction was monitored by TLC and LCMS. After consumption of starting material, the reaction mixture was quenched with ice water (50 mL) and extracted with EtOAc (2×100 mL). Combined organic layers were washed with brine solution (50 mL), dried over Na₂SO₄ and concentrated to get crude. Crude compound was purified by normal phase silica chromatography to afford compound Methyl N²—((S)-2-((((3-chlorobenzyl)oxy)carbonyl)amino)-3-cyclohexylpropanoyl)-N⁵-(2-(ethylsulfonamido)ethyl)-N⁵-methyl-L-glutaminate (8).

### 3-Chlorobenzyl ((13S,16S)-17-cyclohexyl-13-(hydroxymethyl)-9-methyl-10,15-dioxo-3,6-dioxa-9,14-diazaheptadecan-16-yl)carbamate (8)

To a stirred solution of methyl N2-((S)-2-((((3-chlorobenzyl)oxy)carbonyl)amino)-3-cyclohexylpropanoyl)-N5-(2-(ethylsulfonamido)ethyl)-N5-methyl-L-glutaminate (8) (1 g, 1.58 mmol) in DCM (10 mL) at 0° C. was added 2M LiBH₄ in THE (2.3 mL, 4.75 mmol) then reaction mixture was stirred for 2 h at rt. Reaction mixture was quenched with ice water (50 mL) and extracted with DCM (2×20 mL). The combined organic layer was washed with brine solution (50 mL), dried over Na₂SO₄ and concentrated to get crude residue. It was purified by normal phase silica chromatography (100-200 mesh) to afford 3-chlorobenzyl ((S)-3-cyclohexyl-1-(((S)-5-((2-(ethylsulfonamido)ethyl)(methyl)amino)-1-hydroxy-5-oxopentan-2-yl)amino)-1-oxopropan-2-yl)carbamate (9).

### 3-Chlorobenzyl ((S)-3-cyclohexyl-1-(((S)-5-((2-(ethylsulfonamido)ethyl)(methyl)amino)-1,5-dioxopentan-2-yl)amino)-1-oxopropan-2-yl)carbamate A38

To a stirred solution of 3-chlorobenzyl ((S)-3-cyclohexyl-1-(((S)-5-((2-(ethylsulfonamido)ethyl)(methyl)amino)-1-hydroxy-5-oxopentan-2-yl)amino)-1-oxopropan-2-yl)carbamate (9) (150 mg, 0.2487 mmol) was dissolved in DCM (3 mL) at 0° C. was added Dess-Martin periodinane (263 mg, 0.62189 mmol) and stirred at RT for 3 h. Reaction mixture was diluted by DCM (10 mL) and washed with sat. Hypo solution (3×20 mL), sat. NaHCO₃ solution (3×20 mL). Organic layers were dried over anhydrous Na₂SO₄, filtered and concentrated to get the crude compound 3-chlorobenzyl ((S)-3-cyclohexyl-1-(((S)-5-((2 (ethylsulfonamido)ethyl) (methyl)amino)-1,5-dioxopentan-2-yl)amino)-1-oxopropan-2-yl)carbamate A38.

TLC system: 10% Methanol in DCM; Rf: 0.5

LCMS (ESI): m/z 601.26 (M+H)⁺

### 3-Chlorobenzyl((2S)-3-cyclohexyl-1-(((2S)-1-(diethoxyphosphoryl)-5-((2(ethylsulfonamido) ethyl)(methyl)amino)-1-hydroxy-5-oxopentan-2-yl)amino)-1-oxopropan-2-yl)carbamate A22

To a stirred solution of 3-chlorobenzyl ((S)-3-cyclohexyl-1-(((S)-5-((2 (ethylsulfonamido)ethyl)(methyl)amino)-1,5-dioxopentan-2-yl)amino)-1-oxopropan-2-yl)carbamate (180 mg, 0.299 mmol) in DCM (4 mL) was added DIPEA (0.16 mL, 0.898 mmol) and diethyl phosphite (0.120 mL, 0.898 mmol). Then reaction mixture was stirred at RT for 16 h (monitoring by TLC indicated complete disappearance of the starting material) and then reaction mixture was taken in DCM (50 mL) and washed with water (2×50 mL) and brine solution. Organic layer was dried over anhydrous Na₂SO₄, and evaporated to afford crude residue. It was purified by prep HPLC and afforded 3-Chlorobenzyl((2S)-3-cyclohexyl-1-(((2S)-1-(diethoxyphosphoryl)-5-((2(ethylsulfonamido) ethyl)(methyl)amino)-1-hydroxy-5-oxopentan-2-yl)amino)-1-oxopropan-2-yl)carbamate A22.

TLC system: 10% MeOH in DCM; Rf: 0.4

LCMS (ESI): m/z 739.28 (M+H)⁺

### N²—((S)-2-((((3-chlorobenzyl)oxy)carbonyl)amino)-3-cyclohexylpropanoyl)-N⁵-(2-(ethylsulfonamido)ethyl)-N⁵-methyl-L-glutamine (10)

To a stirred solution of methyl N²—((S)-2-((((3-chlorobenzyl)oxy)carbonyl)amino)-3-cyclohexylpropanoyl)-N⁵-(2-(ethylsulfonamido)ethyl)-N⁵-methyl-L-glutaminate (8) (300 mg, 0.4761 mmol) in MeOH (3.0 mL) and water (3.0 mL), was added lithium hydroxide (34 mg, 1.4285 mmol) at RT and stirred at room temperature for 3 h. The progress of the reaction was monitored by TLC and LCMS. After 3 h, the reaction mixture completely distilled under reduced pressure, crude compound acidified with 1 N HCl solution up to pH ˜3-4, and extracted with DCM (2×20 mL), combined organic layers were dried over sodium sulfate, and concentrated under reduced pressure to afford crude compound N²—((S)-2-((((3-chlorobenzyl)oxy)carbonyl)amino)-3-cyclohexylpropanoyl)-N⁵-(2-(ethylsulfonamido)ethyl)-N5-methyl-L-glutamine (10).

### 3-Chlorobenzyl ((S)-3-cyclohexyl-1-(((S)-5-((2-(ethylsulfonamido)ethyl)(methyl)amino)-1-(methoxy(methyl)amino)-1,5-dioxopentan-2-yl)amino)-1-oxopropan-2-yl)carbamate A29

To a stirred solution of N²—((S)-2-((((3-chlorobenzyl)oxy)carbonyl)amino)-3-cyclohexylpropanoyl)-N⁵-(2-(ethylsulfonamido)ethyl)-N5-methyl-L-glutamine (10) (200 mg, 0.3246 mmol) in THE (2.0 mL) and DCM (2.0 mL), was added EDC.HCl (93 mg, 0.48699 mmol), HOBT (65 mg, 0.48699 mmol), N-methylmorpholine (0.042 mL, 0.3896 mmol) and N,O-dimethylhydroxylamine (37 mg, 0.3896 mmol) subsequently at 0° C. The resulting solution was stirred at room temperature for 16 h. The progress of the reaction was monitored by TLC and LCMS. After 16 h, the reaction mixture was diluted with ice water (10 mL), extracted with DCM (2×20 mL). The combined organic layers were dried over sodium sulfate, filtered and evaporated under reduced pressure. The crude residue was purified by prep HPLC and afforded 3-chlorobenzyl ((S)-3-cyclohexyl-1-(((S)-5-((2-(ethylsulfonamido)ethyl)(methyl)amino)-1-(methoxy(methyl)amino)-1,5-dioxopentan-2-yl)amino)-1-oxopropan-2-yl)carbamate A29.

TLC system: 10% Methanol in DCM; Rf: 0.65

LCMS (ESI): m/z 660.59 (M+H)⁺

### 3-Chlorobenzyl ((S)-3-cyclohexyl-1-(((S)-5-((2-(ethylsulfonamido)ethyl)(methyl)amino)-1,5-dioxopentan-2-yl)amino)-1-oxopropan-2-yl)carbamate A38

To a stirred solution of 3-chlorobenzyl ((S)-3-cyclohexyl-1-(((S)-5-((2-(ethylsulfonamido)ethyl)(methyl)amino)-1-(methoxy(methyl)amino)-1,5-dioxopentan-2-yl)amino)-1-oxopropan-2-yl)carbamate A29 (150 mg, 0.22761 mmol) in THE (1.5 mL) was added 2.4M LiAlH₄ in THE (0.14 mL, 0.341 mmol) at −20° C. and the reaction mixture was allowed to RT and stirred for 2 h. The progress of the reaction was monitored by TLC and LCMS. After 2 h, the reaction mixture was diluted with ice water (10 mL) and extracted with ethyl acetate (2×20 mL), combined organic layers were washed brine solution (10 mL) and dried over sodium sulfate, filtered and evaporated under reduced pressure. The crude reaction mixture was purified by prep-HPLC to afforded 3-chlorobenzyl ((S)-3-cyclohexyl-1-(((S)-5-((2-(ethylsulfonamido)ethyl)(methyl)amino)-1,5-dioxopentan-2-yl)amino)-1-oxopropan-2-yl)carbamate A38.

TLC system: 10% Methanol in DCM; Rf: 0.5

LCMS (ESI): m/z 601.42 (M+H)⁺

### Synthesis of Compounds A105, A47, and A39

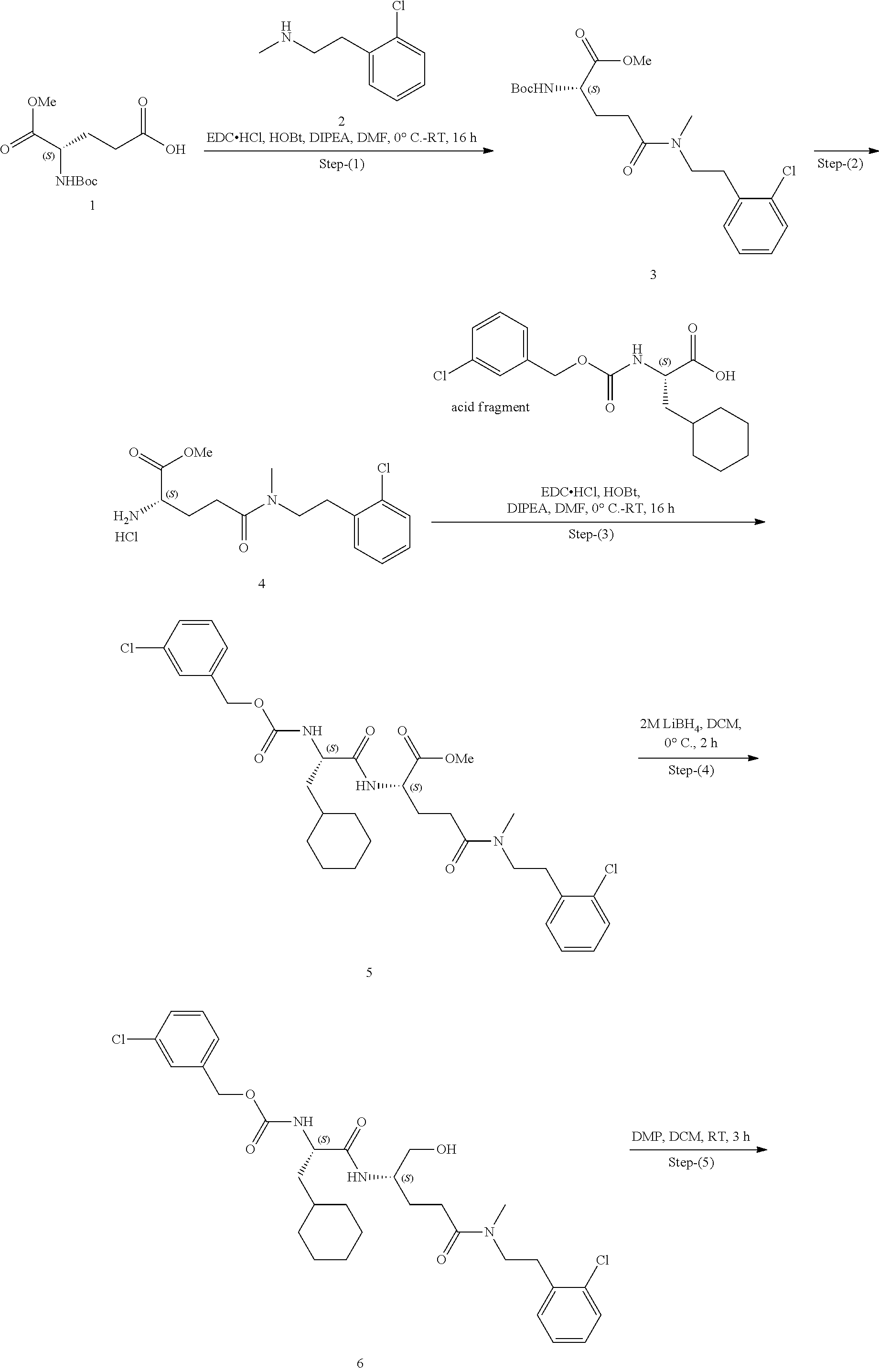

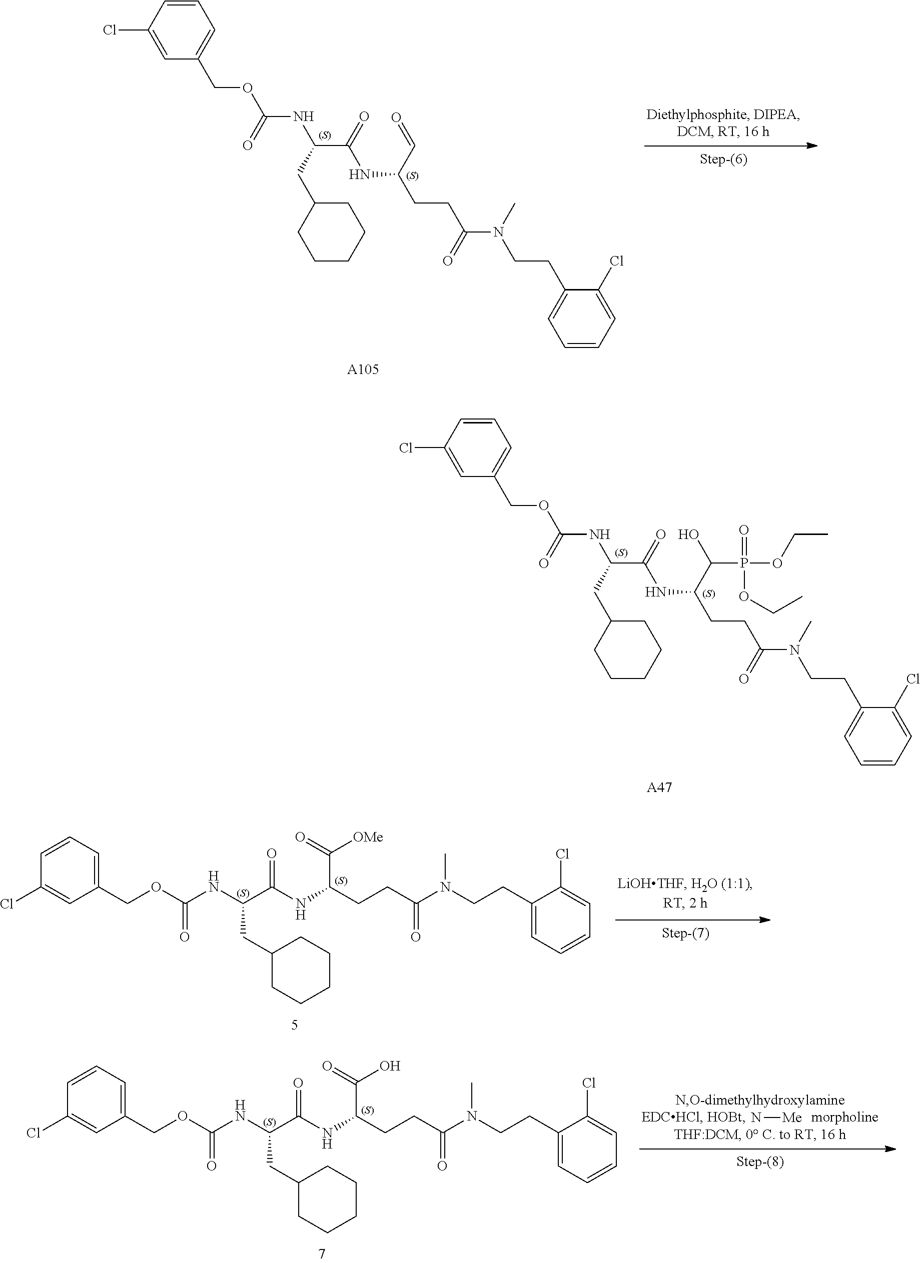

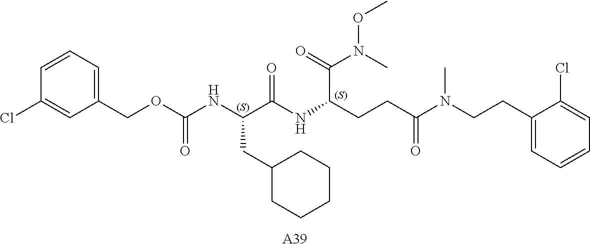

### Methyl N2-(tert-butoxycarbonyl)-N5-(2-chlorophenethyl)-N5-methyl-L-glutaminate (3)

To a stirred solution of (S)-4-((tert-butoxycarbonyl)amino)-5-methoxy-5-oxopentanoic acid (1) (2 g, 7.662 mmol) in DMF (20 mL) was added EDC.HCl (2.19 g, 11.494 mmol), HOBT (1.55 g, 11.494 mmol), DIPEA (4.2 mL, 22.98 mmol) and 2-(2-chlorophenyl)-N-methylethan-1-amine (2) (1.56 g, 9.195 mmol) at 0° C. simultaneously and stirred at room temperature for 16 h. The progress of the reaction was monitored by TLC and LCMS. After 16 h, the reaction mixture was diluted with ice water (50 mL), extracted with ethyl acetate (2×50 mL), dried over sodium sulfate and evaporated under reduced pressure. The crude residue was purified by silica gel column by eluting with 80% ethyl acetate in pet ether to afford methyl N2-(tert-butoxycarbonyl)-N5-(2-chlorophenethyl)-N5-methyl-L-glutaminate (3).

### Methyl N5-(2-chlorophenethyl)-N5-methyl-L-glutaminate hydrochloride (4)

To a stirred solution of methyl N2-(tert-butoxycarbonyl)-N5-(2-chlorophenethyl)-N5-methyl-L-glutaminate (3) (1 g, 2.427 mmol) in 1,4-dioxane (10 mL) was added 4N HCl in dioxane (10 mL) drop wise at 0° C. Reaction mixture was stirred at RT for 2 h. Progress of the reaction was monitored by TLC. After consumption of starting material, the reaction mixture was evaporated under reduced pressure to obtained crude compound, the resulting crude was triturated with diethyl ether to afford methyl N5-(2-chlorophenethyl)-N5-methyl-L-glutaminate hydrochloride (4).

### Methyl N2-((S)-2-((((3-chlorobenzyl)oxy)carbonyl)amino)-3-cyclohexylpropanoyl)-N5-(2-chlorophenethyl)-N5-methyl-L-glutaminate (5)

To a stirred solution of (S)-2-((((3-chlorobenzyl)oxy)carbonyl)amino)-3-cyclohexylpropanoic acid (1 g, 2.949 mmol) in DMF (10 mL) was added EDC.HCl (0.840 g, 4.424 mmol), HOBT (0.590 g, 4.424 mmol), DIPEA (1.6 mL, 8.849 mmol) and methyl N5-(2-chlorophenethyl)-N5-methyl-L-glutaminate hydrogen chloride (4) (1.23 g, 3.139 mmol) at 0° C. simultaneously and stirred at room temperature for 16 h. Progress of the reaction was monitored by TLC and LCMS. Reaction mixture was diluted with ice water (30 mL), extracted with ethyl acetate (2×30 mL), the combined organic layer was dried over sodium sulfate and evaporated under reduced pressure. The crude residue was purified by silica gel column by eluting with 80% ethyl acetate in pet ether to afford methyl N2-((S)-2-((((3-chlorobenzyl) oxy) carbonyl) amino)-3-cyclohexylpropanoyl)-N5-(2-chlorophenethyl)-N5-methyl-L-glutaminate (5).

### 3-Chlorobenzyl ((S)-1-(((S)-5-((2-chlorophenethyl)(methyl)amino)-1-hydroxy-5-oxopentan-2-yl)amino)-3-cyclohexyl-1-oxopropan-2-yl)carbamate (6)

To a stirred solution of methyl N2-((S)-2-((((3-chlorobenzyl) oxy) carbonyl) amino)-3-cyclohexylpropanoyl)-N5-(2-chlorophenethyl)-N5-methyl-L-glutaminate (5) (0.31 g, 0.489 mmol) in DCM (5 mL) was added 2M LiBH₄ in THE (0.73 mL, 1.468 mmol) at 0° C. and reaction mixture was stirred for 2 h at 0° C. The progress of the reaction was monitored by TLC and LCMS. Reaction mixture was quenched with water (10 mL) and extracted with DCM (2×15 mL). Organic layer was washed with brine solution (15 mL), dried over Na₂SO₄ and concentrated to get the crude product. The crude product was purified by silica gel column by eluting with 100% ethyl acetate in pet ether to afford 3-chlorobenzyl ((S)-1-(((S)-5-((2-chlorophenethyl) (methyl)amino)-1-hydroxy-5-oxopentan-2-yl)amino)-3-cyclohexyl-1-oxopropan-2-yl)carbamate (6).

### 3-Chlorobenzyl ((S)-1-(((S)-5-((2-chlorophenethyl)(methyl)amino)-1,5-dioxopentan-2-yl)amino)-3-cyclohexyl-1-oxopropan-2-yl)carbamate A105

To a stirred solution of 3-chlorobenzyl ((S)-1-(((S)-5-((2-chlorophenethyl) (methyl)amino)-1-hydroxy-5-oxopentan-2-yl)amino)-3-cyclohexyl-1-oxopropan-2-yl)carbamate (6) (150 mg, 0.247 mmol) in DCM (10 mL) was added Dess-Martin periodinane (314 mg, 0.741 mmol) at 0° C. and stirred at RT for 3 h. The progress of the reaction was monitored by TLC and LCMS. Reaction mixture was diluted with DCM (10 mL) and washed with sat. Hypo solution (3×20 mL), sat. NaHCO₃ solution (3×20 mL). Organic layer was dried over anhydrous Na₂SO₄, filtered and concentrated to get crude compound. The crude compound was purified by prep HPLC to afford 3-chlorobenzyl ((S)-1-(((S)-5-((2-chlorophenethyl) (methyl) amino)-1, 5-dioxopentan-2-yl) amino)-3-cyclohexyl-1-oxopropan-2-yl) carbamate A105.

TLC system: 5% Methanol in DCM; Rf: 0.5

LCMS (ESI): m/z 604.51 (M+H)⁺

### 3-Chlorobenzyl ((2S)-1-(((2S)-5-((2-chlorophenethyl)(methyl)amino)-1-(diethoxyphosphoryl)-1-hydroxy-5-oxopentan-2-yl)amino)-3-cyclohexyl-1-oxopropan-2-yl)carbamate A47

To a stirred solution of 3-chlorobenzyl ((S)-1-(((S)-5-((2-chlorophenethyl) (methyl) amino)-1, 5-dioxopentan-2-yl) amino)-3-cyclohexyl-1-oxopropan-2-yl) carbamate A105 (230 mg, 0.380 mmol) was dissolved in DCM (10 mL), were added DIPEA (0.21 mL, 1.141 mmol) and diethylphosphite (0.15 mL, 1.141 mmol) at 0° C. and stirred at RT for 16 h. The progress of the reaction was monitored by TLC and LCMS. Reaction mixture was diluted with H₂O (15 mL) and extracted with DCM (2×15 mL), dried over anhydrous Na₂SO₄, crude compound purified by prep-HPLC afford 3-chlorobenzyl ((2S)-1-(((2S)-5-((2-chlorophenethyl)(methyl)amino)-1-(diethoxyphosphoryl)-1-hydroxy-5-oxopentan-2-yl)amino)-3-cyclohexyl-1-oxopropan-2-yl)carbamate A47.

TLC system: 10% Methanol in DCM; Rf: 0.3

LCMS (ESI): m/z 719.54 (M+H)⁺

### N2-((S)-2-((((3-chlorobenzyl) oxy) carbonyl) amino)-3-cyclohexylpropanoyl)-N5-(2-chlorophenethyl)-N5-methyl-L-glutamine (6)

To a stirred solution of methyl N2-((S)-2-((((3-chlorobenzyl)oxy)carbonyl)amino)-3-cyclohexylpropanoyl)-N5-(2-chlorophenethyl)-N5-methyl-L-glutaminate (5) (300 mg, 0.473 mmol) in THE (3 mL) and water (3 mL), was added lithium hydroxide (0.022 g, 0.947 mmol) and stirred at room temperature for 2 h. The progress of the reaction was monitored by TLC and LCMS. Reaction mixture completely distilled under reduced pressure, crude compound acidified with 2N HCL solution up to pH ˜4, and extracted with ethyl acetate (2×10 mL), dried over sodium sulfate, concentrated under reduced pressure to afford crude compound N2-((S)-2-((((3-chlorobenzyl)oxy)carbonyl)amino)-3-cyclohexylpropanoyl)-N5-(2-chlorophenethyl)-N5-methyl-L-glutamine (6).

### 3-Chlorobenzyl ((S)-1-(((S)-5-((2-chlorophenethyl)(methyl)amino)-1-(methoxy(methyl)amino)-1,5-dioxopentan-2-yl)amino)-3-cyclohexyl-1-oxopropan-2-yl)carbamate A39

To a stirred solution of N2-((S)-2-((((3-chlorobenzyl)oxy)carbonyl)amino)-3-cyclohexylpropanoyl)-N5-(2-chlorophenethyl)-N5-methyl-L-glutamine (6) (250 mg, 0.403 mmol) in tetrahydrofuran (5 mL) and DCM (5 mL) was added EDC.HCl (92 mg, 0.484 mmol), HOBT (65 mg, 0.484 mmol), N-methylmorpholine (48 mg, 0.484 mmol) and N,O-dimethylhydroxylamine (47 mg, 0.484 mmol) at 0° C. simultaneously and stirred at room temperature for 16 h. The progress of the reaction was monitored by TLC and LCMS. Reaction mixture was diluted with ice water (20 mL), extracted with ethyl acetate (2×20 mL), dried over sodium sulfate and evaporated under reduced pressure. The crude residue was purified by prep-HPLC to offer 3-chlorobenzyl((S)-1-(((S)-5-((2-chlorophenethyl)(methyl)amino)-1-(methoxy(methyl)amino)-1,5-dioxopentan-2-yl)amino)-3-cyclohexyl-1-oxopropan-2-yl)carbamate A39.

TLC system: 10% Methanol in DCM; Rf: 0.4

LCMS (ESI): m/z 663.57 (M+H)⁺

### Synthesis of Compounds A48, A40, and A41

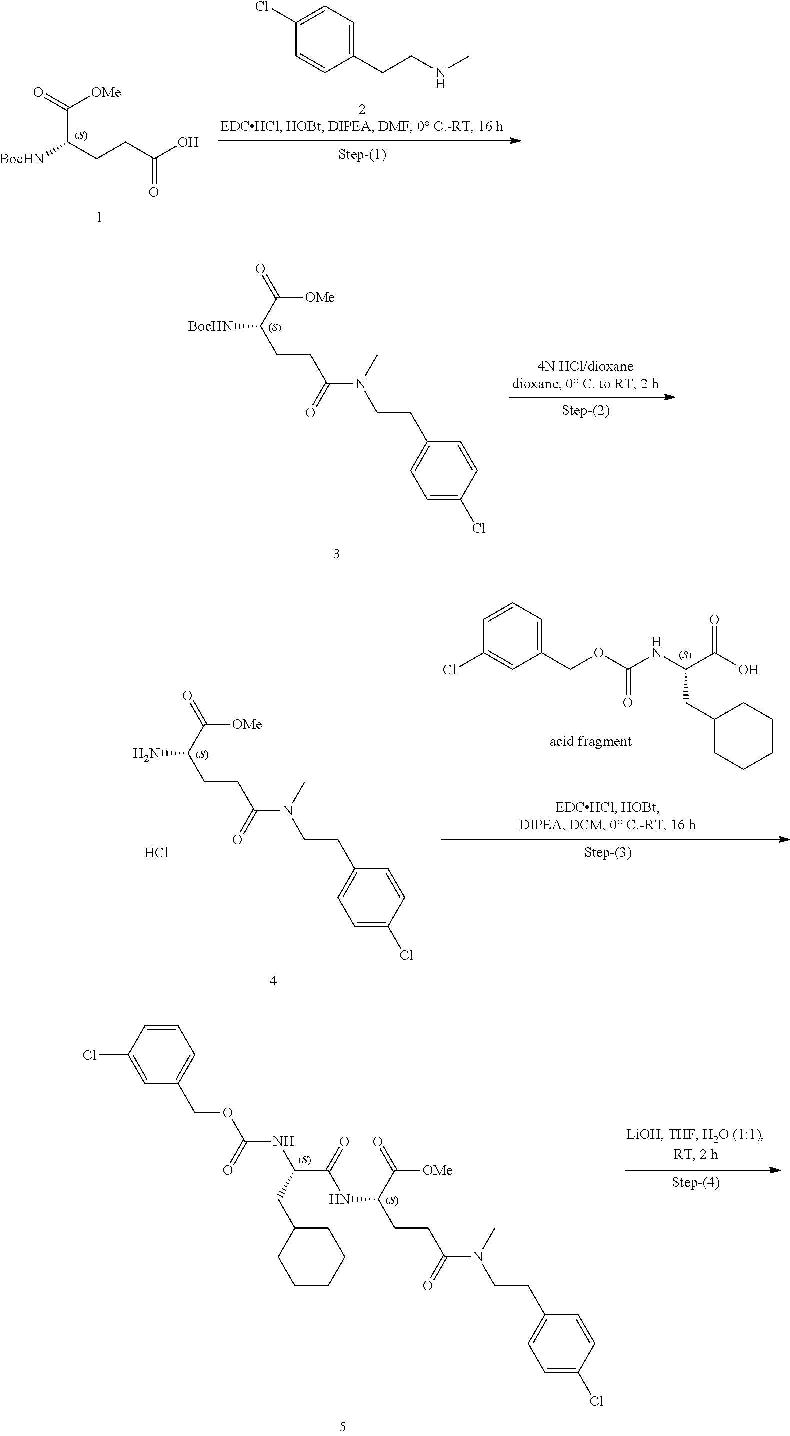

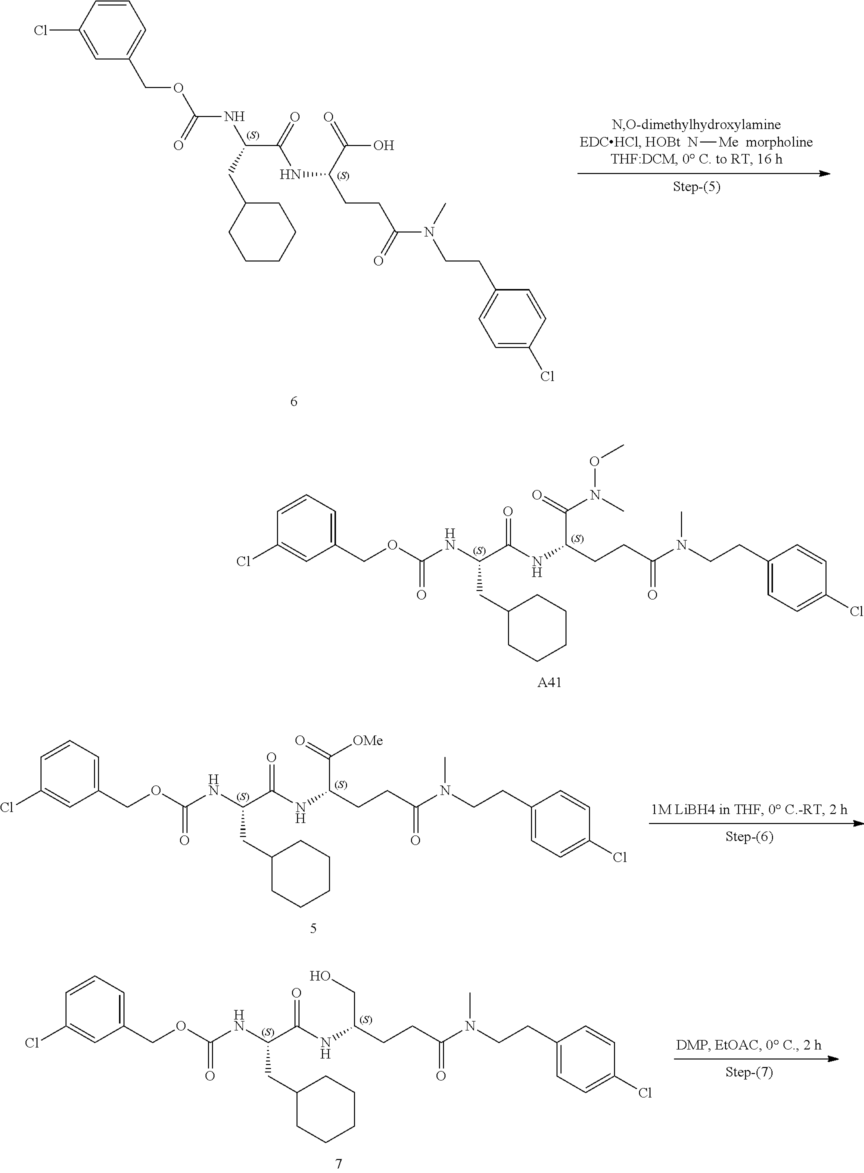

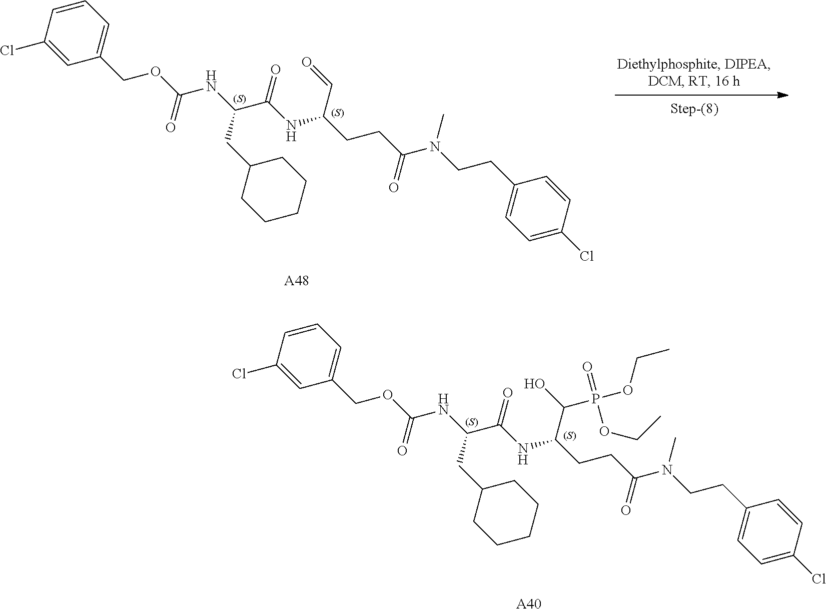

### Methyl N2-(tert-butoxycarbonyl)-N5-(4-chlorophenethyl)-N5-methyl-L-glutaminate (3)

To a stirred solution of (S)-4-((tert-butoxycarbonyl)amino)-5-methoxy-5-oxopentanoic acid (1) (2 g, 7.662 mmol) in DMF (20 mL) were added EDC.HCl (2.1 g, 11.49 mmol), HOBT (1.55 g, 11.49 mmol), DIPEA (4.2 mL, 22.98 mmol) and 2-(4-chlorophenyl)-N-methylethan-1-amine (2) (1.295 g, 7.662 mmol) at 0° C. and the reaction mixture was stirred at RT for 16 h. The progress of the reaction was monitored by TLC and LCMS. After 16 h, the reaction mixture was diluted with ice water (20 mL), extracted with ethyl acetate (3×20 mL). The combined organic layer was dried over sodium sulfate, filtered and evaporated under reduced pressure. The crude residue was purified by combi-flash compound eluted at 40% Ethyl acetate in pet ether to afford methyl methyl N2-(tert-butoxycarbonyl)-N5-(4-chlorophenethyl)-N5-methyl-L-glutaminate (3).

### Methyl N5-(4-chlorophenethyl)-N5-methyl-L-glutaminate hydrochloride (4)

To a stirred solution of methyl N2-(tert-butoxycarbonyl)-N5-(4-chlorophenethyl)-N5-methyl-L-glutaminate (3) (2 g, 4.854 mmol) in 1,4-dioxane (10 mL) was added 4N HCl in dioxane (10 mL) with drop wise at 0° C. and the reaction mixture was stirred at RT for 3 h. The progress of the reaction was monitored by TLC. After consumption of starting material, the reaction mixture was evaporated under reduced pressure to obtained crude compound, the resulting crude triturated with n-pentane (10 mL) to afford methyl N5-(4-chlorophenethyl)-N5-methyl-L-glutaminate hydrochloride (4).

### Methyl N2-((S)-2-((((3-chlorobenzyl) oxy) carbonyl) amino)-3-cyclohexylpropanoyl)-N5-(4-chlorophenethyl)-N5-methyl-L-glutaminate (5)

To a stirred solution of (S)-2-((((3-chlorobenzyl)oxy)carbonyl)amino)-3-cyclohexylpropanoic acid (acid fragment) (1.1 g, 3.53 mmol) in DMF (10 mL) added EDC.HCl (842 mg, 4.41 mmol), HOBt (595 mg, 4.41 mmol), DIPEA (1.6 mL, 8.82 mmol) and methyl N5-(4-chlorophenethyl)-N5-methyl-L-glutaminate hydrochloride (4) (1 g, 2.94 mmol) at 0° C. simultaneously and stirred at room temperature for 16 h. The progress of the reaction was monitored by TLC and LCMS. After 16 h, reaction mixture was diluted with ice water (20 mL), extracted with ethyl acetate (3×30 mL). The combined organic layer was dried over sodium sulfate, filtered and evaporated under reduced pressure. The crude residue was purified by combi-flash, compound eluted at 70% ethyl acetate in pet ether to afford methyl N2-((S)-2-((((3-chlorobenzyl)oxy)carbonyl)amino)-3-cyclohexylpropanoyl)-N5-(4-chlorophenethyl)-N5-methyl-L-glutaminate (5).

### N2-((S)-2-((((3-chlorobenzyl)oxy)carbonyl)amino)-3-cyclohexylpropanoyl)-N5-(4-chlorophenethyl)-N5-methyl-L-glutamine (6)

To a stirred solution of methyl N2-((S)-2-((((3-chlorobenzyl)oxy)carbonyl) amino)-3-cyclohexylpropanoyl)-N5-(4-chlorophenethyl)-N5-methyl-L-glutaminate (5) (300 mg, 0.473 mmol) in THE (2 mL) and water (2 mL) was added lithium hydroxide (34.1 mg, 1.42 mmol) at 0° C. and stirred at RT for 2 h. The progress of the reaction was monitored by TLC and LCMS. After 2 h, the reaction mixture completely distilled under reduced pressure, crude compound acidified with potassium bisulfate solution up to pH ˜4, and extracted with ethyl acetate (3×10 mL), dried over sodium sulfate, concentrated under reduced pressure to afford crude compound. The crude residue was purified by combi-flash, compound eluted at 90% ethyl acetate in pet ether to afford N2-((S)-2-((((3-chlorobenzyl)oxy) carbonyl) amino)-3-cyclohexylpropanoyl)-N5-(4-chlorophenethyl)-N5-methyl-L-glutamine(6).

### 3-Chlorobenzyl ((S)-1-(((S)-5-((4-chlorophenethyl)(methyl)amino)-1-(methoxy(methyl)amino)-1,5-dioxopentan-2-yl)amino)-3-cyclohexyl-1-oxopropan-2-yl)carbamate A41

To a stirred solution of N2-((S)-2-((((3-chlorobenzyl)oxy)carbonyl) amino)-3-cyclohexylpropanoyl)-N5-(4-chlorophenethyl)-N5-methyl-L-glutamine (6) (280 mg, 0.452 mmol) in THE (2.5 mL) and DCM (2.5 mL), added EDC.HCl (73 mg, 0.542 mmol), HOBt (103.5 mg, 0.542 mmol), N-methylmorpholine (0.1 mL, 0.542 mmol) and N,O-dimethylhydroxylamine (53.8 mg, 0.542 mmol) at 0° C. simultaneously and stirred at room temperature for 16 h. The progress of the reaction was monitored by TLC and LCMS. Then the reaction mixture was diluted with ice water (10 mL), extracted with ethyl acetate (3×15 mL). The combined organic layer was dried over sodium sulfate, filtered and evaporated under reduced pressure. The crude residue was purified by Prep-HPLC to afford 3-chlorobenzyl ((S)-1-(((S)-5-((4-chlorophenethyl)(methyl) amino)-1-(methoxy(methyl)amino)-1,5-dioxopentan-2-yl)amino)-3-cyclohexyl-1-oxopropan-2-yl)carbamate A41.

TLC system: 10% Methanol in DCM; Rf: 0.6

LCMS (ESI): m/z 663.57 (M+H)⁺

### 3-Chlorobenzyl ((S)-1-(((S)-5-((4-chlorophenethyl)(methyl)amino)-1-hydroxy-5-oxopentan-2-yl)amino)-3-cyclohexyl-1-oxopropan-2-yl)carbamate (7)

To a stirred solution of methyl N2-((S)-2-((((3-chlorobenzyl)oxy)carbonyl)amino)-3-cyclohexylpropanoyl)-N5-(4-chlorophenethyl)-N5-methyl-L-glutaminate (5) (400 mg, 0.631 mmol) in DCM (10 mL) was added 2M LiBH₄ in THE (0.90 mL, 1.893 mmol) at 0° C. and reaction mixture was stirred at room temperature for 2 h. The progress of the reaction was monitored by TLC and LCMS. After consumption of starting material, the reaction mixture was quenched with ammonium chloride solution (10 mL), extracted with DCM (2×10 mL). The combined organic layer was washed with water (2×10 mL), and brine (10 mL), organic layer was dried over sodium sulfate, filtered and evaporated under reduced pressure. The crude residue was purified by combi-flash, compound eluted at 5% methanol in DCM afforded to 3-chlorobenzyl ((S)-1-(((S)-5-((4-chlorophenethyl)(methyl)amino)-1-hydroxy-5-oxopentan-2-yl)amino)-3-cyclohexyl-1-oxopropan-2-yl)carbamate(7).

### 3-Chlorobenzyl ((S)-1-(((S)-5-((4-chlorophenethyl)(methyl)amino)-1,5-dioxopentan-2-yl)amino)-3-cyclohexyl-1-oxopropan-2-yl)carbamate A48

To a stirred solution of 3-chlorobenzyl((S)-1-(((S)-5-((4-chlorophenethyl) (methyl)amino)-1-hydroxy-5-oxopentan-2-yl)amino)-3-cyclohexyl-1-oxopropan-2-yl) carbamate (7) (150 mg, 0.244 mmol) was dissolved in Ethyl acetate (5 mL) was added Dess-Martin periodinane (315 mg, 0.743 mmol) at 0° C. and stirred at RT for 3 h. The progress of the reaction was monitored by TLC and LCMS. After 3 h, the reaction mixture was diluted with ethyl acetate (10 mL) and filtered filtrate was washed with sat. Hypo solution (3×10 mL), sat. NaHCO₃ solution (3×10 mL). Organic layer was dried over anhydrous Na₂SO₄, filtered and concentrated to get crude compound. The crude compound was purified by prep HPLC to afford 3-chlorobenzyl ((S)-1-(((S)-5-((4-chlorophenethyl) (methyl) amino)-1, 5-dioxopentan-2-yl) amino)-3-cyclohexyl-1-oxopropan-2-yl) carbamate A48.

TLC system: 70% Ethyl acetate in Pet ether; Rf: 0.4

LCMS (ESI): m/z 604.50 (M+H)⁺

### 3-Chlorobenzyl ((2S)-1-(((2S)-5-((4-chlorophenethyl)(methyl)amino)-1-(diethoxyphosphoryl)-1-hydroxy-5-oxopentan-2-yl)amino)-3-cyclohexyl-1-oxopropan-2-yl)carbamate A40

To a stirred solution of 3-chlorobenzyl ((S)-1-(((S)-5-((4-chlorophenethyl) (methyl) amino)-1, 5-dioxopentan-2-yl) amino)-3-cyclohexyl-1-oxopropan-2-yl) carbamate A48 (130 mg, 0.215 mmol) in DCM (10 mL) added DIPEA (0.119 mL, 0.646 mmol) followed by diethylphosphite (89 mg, 0.646 mmol) and the reaction mixture stirred at RT for 16 h. The progress of the reaction was monitored by TLC and LCMS. After 16 h, the reaction mixture was quenched with ice water (15 mL) and extracted with DCM (3×10 mL). Combined organic layer was dried over anhydrous Na₂SO₄, and evaporated to afford crude residue. It was purified by prep HPLC to afford 3-chlorobenzyl ((2S)-1-(((2S)-5-((4-chlorophenethyl)(methyl)amino)-1-(diethoxyphosphoryl)-1-hydroxy-5-oxopentan-2-yl) amino)-3-cyclohexyl-1-oxopropan-2-yl)carbamate A40.

TLC system: 50% Ethyl acetate in pet-ether (2 times); Rf: 0.3

LCMS (ESI): m/z 742.47 (M+H)⁺

### Synthesis of Compounds A56, A50, and A42

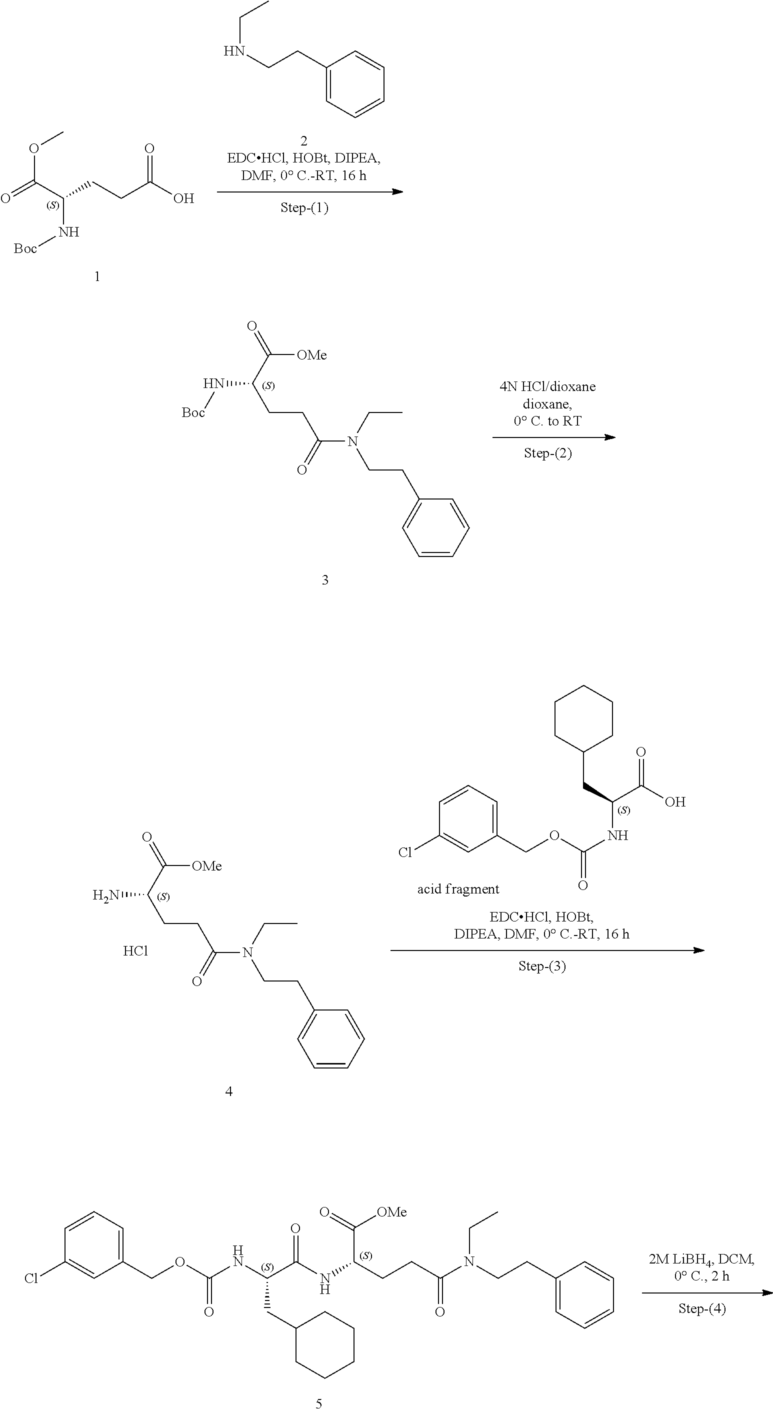

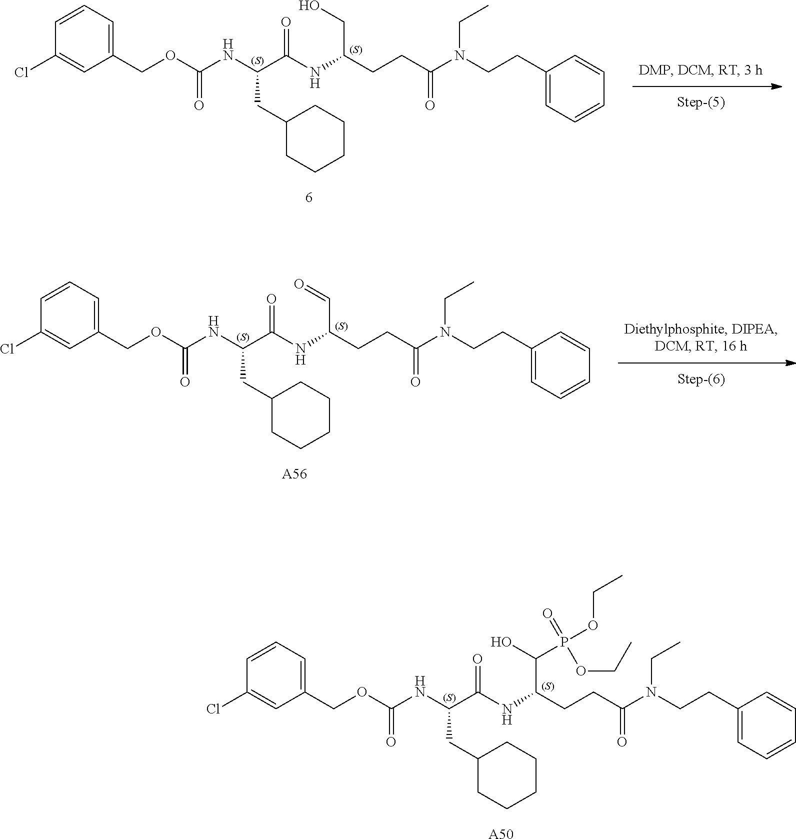

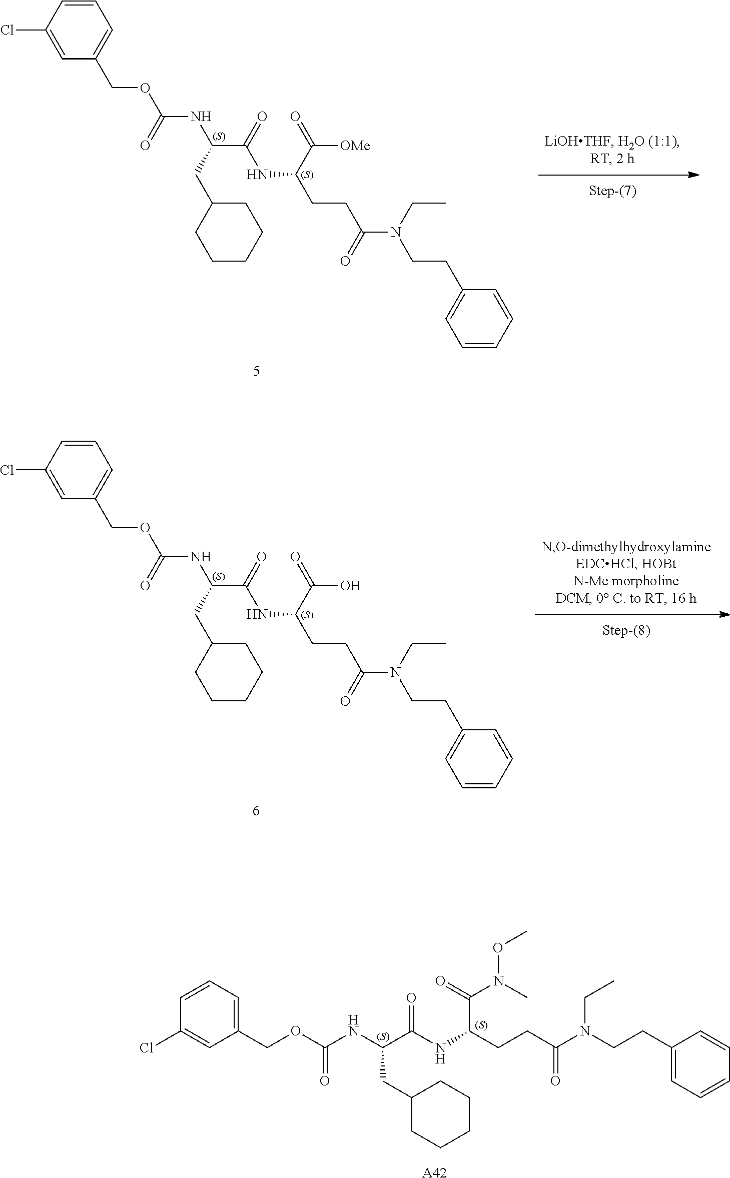

### Methyl N2-(tert-butoxycarbonyl)-N5-ethyl-N5-phenethyl-L-glutaminate (3)

To a stirred solution of (S)-4-((tert-butoxycarbonyl)amino)-5-methoxy-5-oxopentanoic acid (1) (2.5 g, 9.5785 mmol) in DMF (30 mL) were added EDC.HCl (2.74 g, 14.3678 mmol), HOBT (1.93 g, 14.3678 mmol), DIPEA (5.2 mL, 28.7356 mmol) and N-ethyl-2-phenylethan-1-amine (2) (2.12 g, 11.4942 mmol) at 0° C. and the reaction mixture was stirred at RT for 16 h. The progress of the reaction was monitored by TLC and LCMS. After 16 h, the reaction mixture was diluted with ice water (50 mL), extracted with ethyl acetate (2×50 mL). The combined organic layer was dried over sodium sulfate, filtered and evaporated under reduced pressure. The crude residue was purified by combi-flash compound eluted at 60% Ethyl acetate in pet ether to afford methyl N2-(tert-butoxycarbonyl)-N5-ethyl-N5-phenethyl-L-glutaminate (3).

### Methyl N5-ethyl-N5-phenethyl-L-glutaminate hydrochloride (4)

To a stirred solution of methyl N2-(tert-butoxycarbonyl)-N5-ethyl-N5-phenethyl-L-glutaminate (3) (2.2 g, 5.6122 mmol) in 1,4-dioxane (20 mL) was added 4N HCl in dioxane (25 mL) with drop wise at 0° C. and the reaction mixture was stirred at RT for 2 h. The progress of the reaction was monitored by TLC and LCMS. After consumption of starting material, the reaction mixture was evaporated under reduced pressure to obtained crude compound, the resulting crude triturated with diethyl ether to afford methyl N5-ethyl-N5-phenethyl-L-glutaminate hydrochloride (4).

### Methyl N2-((S)-2-((((3-chlorobenzyl)oxy)carbonyl)amino)-3-cyclohexylpropanoyl)-N5-ethyl-N5-phenethyl-L-glutaminate (5)

To a stirred solution of (S)-2-((((3-chlorobenzyl)oxy)carbonyl)amino)-3-cyclohexylpropanoic acid (acid fragment) (1.3 g, 3.8348 mmol) in DMF (20 mL) was added EDC.HCl (1.1 g, 5.7522 mmol), HOBT (0.776 g, 5.7522 mmol), DIPEA (2.1 mL, 10.6508 mmol) and methyl N5-ethyl-N5-phenethyl-L-glutaminate hydrochloride hydrogen chloride (4) (1.34 g, 4.6017 mmol) at 0° C. simultaneously and stirred at room temperature for 16 h. The progress of the reaction was monitored by TLC and LCMS. After 16 h, reaction mixture was diluted with ice water (30 mL), extracted with ethyl acetate (2×30 mL). The combined organic layer was dried over sodium sulfate, filtered and evaporated under reduced pressure. The crude residue was purified by combi-flash, compound eluted at 80% Ethyl acetate in pet ether to afford methyl N2-((S)-2-((((3-chlorobenzyl)oxy)carbonyl)amino)-3-cyclohexylpropanoyl)-N5-ethyl-N5-phenethyl-L-glutaminate (5).

### 3-Chlorobenzyl ((S)-3-cyclohexyl-1-(((S)-5-(ethyl(phenethyl)amino)-1-hydroxy-5-oxopentan-2-yl)amino)-1-oxopropan-2-yl)carbamate (6)

To a stirred solution of methyl N2-((S)-2-((((3-chlorobenzyl)oxy)carbonyl)amino)-3-cyclohexylpropanoyl)-N5-ethyl-N5-phenethyl-L-glutaminate (5) (500 mg, 0.8156 mmol) in DCM (10 mL) was added 2M LiBH₄ in THE (0.8 mL, 1.6313 mmol) at 0° C. and the reaction mixture stirred for 3 h at 0° C. The progress of the reaction was monitored by TLC and LCMS. Then reaction mixture was quenched with water (10 mL) and extracted with DCM (2×10 mL). Organic layer was washed with brine solution (10 mL), dried over Na₂SO₄ and concentrated to get crude compound. It was purified combi-flash, compound eluted at 80% ethyl acetate in pet ether to afford 3-chlorobenzyl ((S)-3-cyclohexyl-1-(((S)-5-(ethyl(phenethyl)amino)-1-hydroxy-5-oxopentan-2-yl)amino)-1-oxopropan-2-yl)carbamate (6).

### 3-Chlorobenzyl ((S)-3-cyclohexyl-1-(((S)-5-(ethyl(phenethyl)amino)-1,5-dioxopentan-2-yl)amino)-1-oxopropan-2-yl)carbamate A56

To a stirred solution of 3-chlorobenzyl ((S)-3-cyclohexyl-1-(((S)-5-(ethyl(phenethyl)amino)-1-hydroxy-5-oxopentan-2-yl)amino)-1-oxopropan-2-yl)carbamate (6) (300 mg, 0.5119 mmol) in DCM (10 mL) was added Dess-Martin periodinane (434 mg, 1.0238 mmol) at 0° C. and stirred at RT for 3 h. The progress of the reaction was monitored by TLC and LCMS. Reaction mixture was diluted with DCM (10 mL) and washed with sat. Hypo solution (3×10 mL), sat. NaHCO₃ solution (3×20 mL). Organic layer was dried over anhydrous Na₂SO₄, filtered and concentrated to get crude compound. The crude compound was prep HPLC to afford 3-chlorobenzyl ((S)-3-cyclohexyl-1-(((S)-5-(ethyl(phenethyl)amino)-1,5-dioxopentan-2-yl)amino)-1-oxopropan-2-yl)carbamate A56.

TLC system: 80% Ethyl acetate in Pet ether; Rf: 0.4

LCMS (ESI): m/z 584.61 (M+H)⁺

### 3-Chlorobenzyl ((2S)-3-cyclohexyl-1-(((2S)-1-(diethoxyphosphoryl)-5-(ethyl(phenethyl)amino)-1-hydroxy-5-oxopentan-2-yl)amino)-1-oxopropan-2-yl)carbamate A50

To a stirred solution of 3-chlorobenzyl ((S)-3-cyclohexyl-1-(((S)-5-(ethyl(phenethyl) amino)-1,5-dioxopentan-2-yl)amino)-1-oxopropan-2-yl)carbamate A56 (200 mg crude, 0.343 mmol) in DCM (5 mL) was added DIPEA (0.2 mL, 0.995 mmol) followed by diethylphosphite (0.13 mL, 0.6861 mmol) and the reaction mixture stirred at RT for 16 h. The progress of the reaction was monitored by TLC and LCMS. After 16 h, the reaction mixture quenched with ammonium chloride (15 mL) and extracted with DCM (2×20 mL). Combined organic layer was dried over anhydrous Na₂SO₄, and evaporated to afford crude residue. It was purified prep HPLC to afford 3-chlorobenzyl ((2S)-3-cyclohexyl-1-(((2S)-1-(diethoxyphosphoryl)-5-(ethyl(phenethyl)amino)-1-hydroxy-5-oxopentan-2-yl)amino)-1-oxopropan-2-yl) carbamate A50.

TLC system: 50% Ethyl acetate in Pet ether; Rf: 0.3

LCMS (ESI): m/z 722.67 (M+H)⁺

### N2-((S)-2-((((3-Chlorobenzyl)oxy)carbonyl)amino)-3-cyclohexylpropanoyl)-N5-ethyl-N5-phenethyl-L-glutamine (6)

To a stirred solution of methyl N2-((S)-2-((((3-chlorobenzyl)oxy)carbonyl)amino)-3-cyclohexylpropanoyl)-N5-ethyl-N5-phenethyl-L-glutaminate (5) (300 mg, 0.4893 mmol) in THE (5 mL) and water (5 mL), was added lithium hydroxide (35 mg, 1.4681 mmol) at RT and stirred at room temperature for 2 h. The progress of the reaction was monitored by TLC and LCMS. After 2 h, the reaction mixture completely distilled under reduced pressure, crude compound acidified with 2N HCL solution up to pH ˜4, and extracted with ethyl acetate (2×10 mL), dried over sodium sulfate, concentrated under reduced pressure to afford N2-((S)-2-((((3-chlorobenzyl)oxy)carbonyl)amino)-3-cyclohexylpropanoyl)-N5-ethyl-N5-phenethyl-L-glutamine (6).

### 3-Chlorobenzyl ((S)-3-cyclohexyl-1-(((S)-5-(ethyl(phenethyl)amino)-1-(methoxy(methyl)amino)-1,5-dioxopentan-2-yl)amino)-1-oxopropan-2-yl)carbamate A42

To a stirred solution of N2-((S)-2-((((3-chlorobenzyl)oxy)carbonyl)amino)-3-cyclohexylpropanoyl)-N5-ethyl-N5-phenethyl-L-glutamine (6) (150 mg, 0.2504 mmol) in THE (3 mL) and DCM (3 mL), added EDC.HCl (71 mg, 0.3756 mmol), HOBT (50 mg, 0.3756 mmol), N-methylmorpholine (0.1 mL, 0.7512 mmol) and N,O-dimethylhydroxylamine (30 mg, 0.3005 mmol) at 0° C. simultaneously and stirred at room temperature for 16 h. The progress of the reaction was monitored by TLC and LCMS. Then the reaction mixture was diluted with ice water (10 mL), extracted with ethyl acetate (2×10 mL). The combined organic layer was dried over sodium sulfate, filtered and evaporated under reduced pressure. The crude residue was purified by prep-HPLC to afforded 3-chlorobenzyl ((S)-3-cyclohexyl-1-(((S)-5-(ethyl (phenethyl) amino)-1-(methoxy(methyl)amino)-1,5-dioxopentan-2-yl)amino)-1-oxopropan-2-yl) carbamate A42.

TLC system: 10% Methanol in DCM; Rf: 0.6

LCMS (ESI): m/z 643.67 (M+H)⁺

### Synthesis of Compound A53, A106, and A43

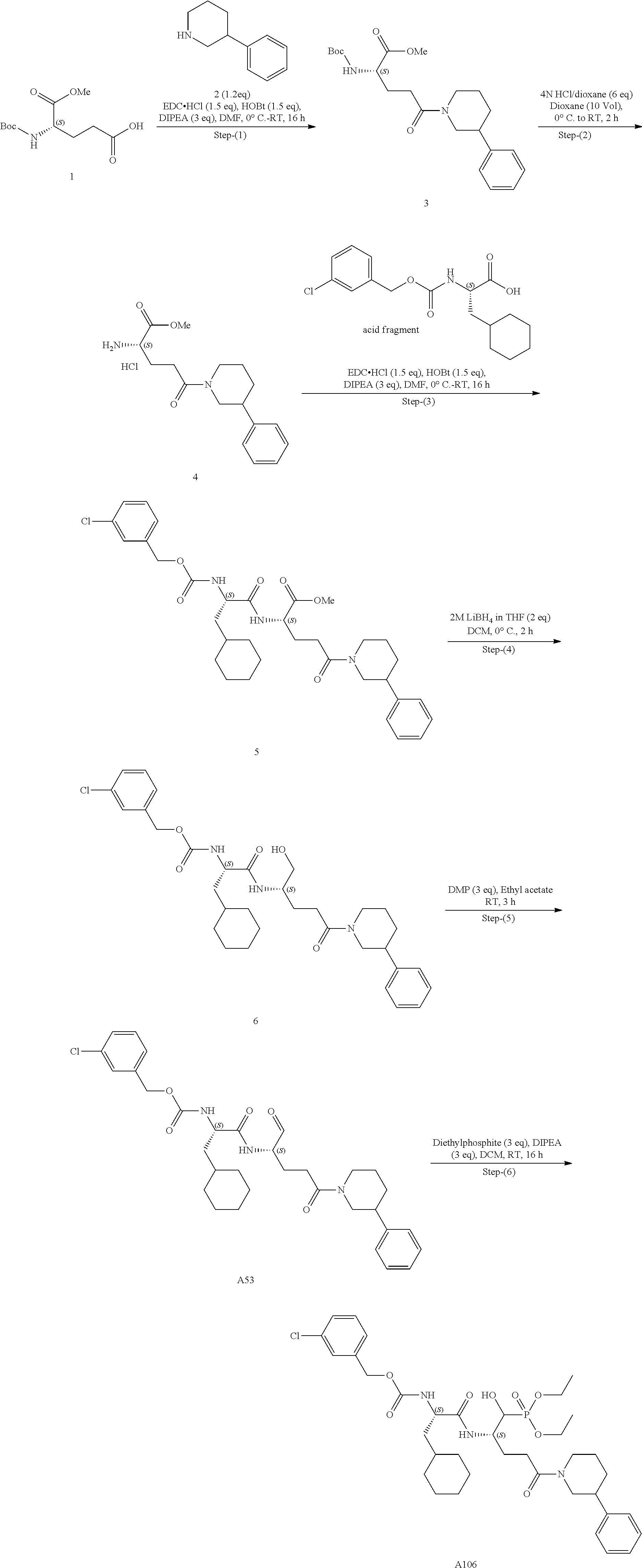

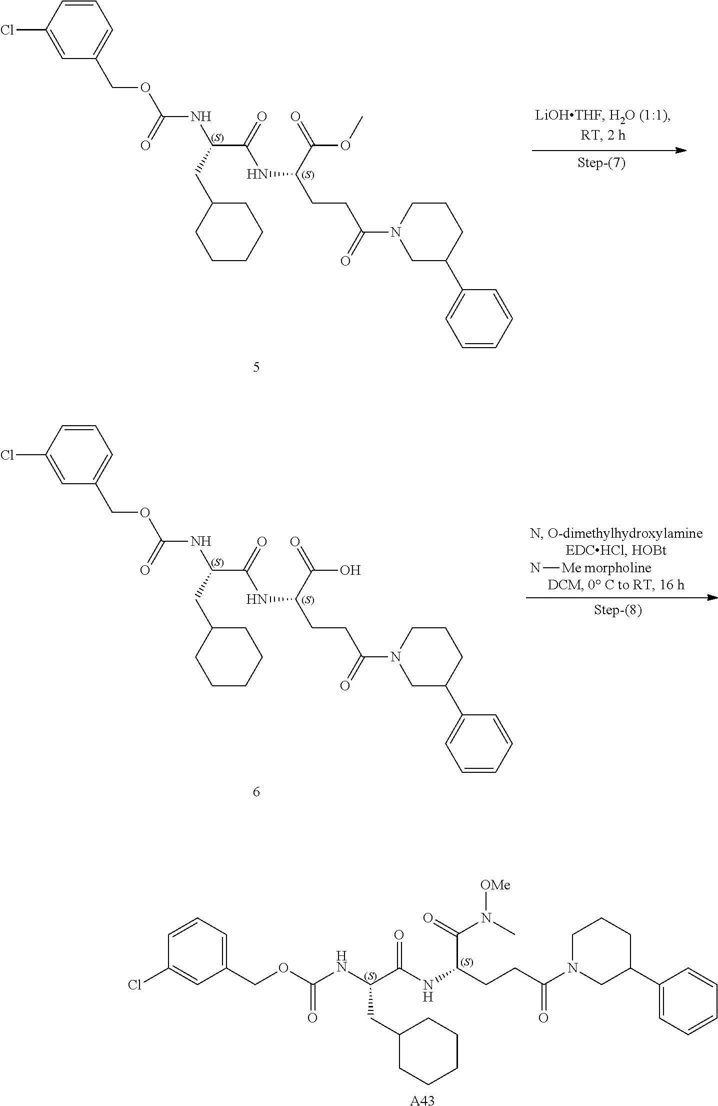

### Methyl (2S)-2-((tert-butoxycarbonyl)amino)-5-oxo-5-(3-phenylpiperidin-1-yl)pentanoate (3)

To a stirred solution of (S)-4-((tert-butoxycarbonyl)amino)-5-methoxy-5-oxopentanoic acid (1) (2.5 g, 9.57 mmol) in DMF (25 mL) was added HOBt (1.9 g, 14.3 mmol), EDC.HCl (2.7 g, 14.3 mmol) and TEA (4 mL, 28.7 mmol) followed by 3-phenylpiperidine (2) (1.85 g, 11.4 mmol) 0° C. Reaction mixture was stirred at room temperature for 16 h. The progress of the reaction was monitored by TLC and LCMS. After consumption of starting material, the reaction mixture was quenched with water (100 mL) and extracted with EtOAc (2×200 mL). Combined organic layers was washed with brine solution (100 mL), dried over Na₂SO₄ and concentrated to get crude residue. Crude compound was purified by normal phase chromatography to afford methyl (2S)-2-((tert-butoxycarbonyl)amino)-5-oxo-5-(3-phenylpiperidin-1-yl)pentanoate (3).

### Methyl (2S)-2-amino-5-oxo-5-(3-phenylpiperidin-1-yl)pentanoate hydrochloride (4)

To a stirred solution of methyl (2S)-2-((tert-butoxycarbonyl)amino)-5-oxo-5-(3-phenylpiperidin-1-yl)pentanoate (3) (2.6 g, 6.4 mmol) in dioxane (30 mL) was added 4 M HCl in dioxane (10 mL) drop wise at 0° C. Reaction mixture was stirred at RT for 2 h. The progress of the reaction was monitored by TLC. After consumption of starting material, the reaction mixture was evaporated under reduced pressure to obtain crude compound, the resulting crude triturated with diethyl ether to afford methyl (2S)-2-amino-5-oxo-5-(3-phenylpiperidin-1-yl)pentanoate hydrochloride (4).

### Methyl (2S)-2-((S)-2-((((3-chlorobenzyl)oxy)carbonyl)amino)-3-cyclohexylpropanamido)-5-oxo-5-(3-phenylpiperidin-1-yl)pentanoate (5)

To a stirred solution of (S)-2-((((3-chlorobenzyl)oxy)carbonyl)amino)-3-cyclohexylpropanoic acid (acid fragment) (1 g, 2.94 mmol) in DMF (10 mL) was added HOBT (595 mg, 4.35 mmol), EDC.HCl (845 mg, 4.35 mmol) and TEA (1.2 mL, 8.84 mmol) followed by methyl N5-methyl-N5-phenethyl-L-glutaminate hydrochloride (4) (1 g, 3.53 mmol) at 0° C. Reaction mixture was stirred at room temperature for 16 h. The progress of the reaction was monitored by TLC. After consumption of starting material, the reaction mixture was quenched with water (10 mL) and extracted with EtOAc (2×100 mL). Combined organic layer was washed with brine solution (50 mL), dried over Na₂SO₄ and concentrated to get crude; Crude compound was purified by normal phase chromatography to afford pure compound methyl (2S)-2-((S)-2-((((3-chlorobenzyl)oxy)carbonyl)amino)-3-cyclohexylpropanamido)-5-oxo-5-(3-phenylpiperidin-1-yl)pentanoate (5).

### 3-Chlorobenzyl ((2S)-3-cyclohexyl-1-(((2S)-1-hydroxy-5-oxo-5-(3-phenylpiperidin-1-yl)pentan-2-yl)amino)-1-oxopropan-2-yl)carbamate (6)

At 0° C., to a stirred solution of methyl (2S)-2-((S)-2-((((3-chlorobenzyl)oxy)carbonyl)amino)-3-cyclohexylpropanamido)-5-oxo-5-(3-phenylpiperidin-1-yl)pentanoate (5) (470 mg, 0.75 mmol) in DCM (10 mL) was added 2M LiBH₄ in THE (1.13 mL, 2.25 mmol) and the reaction mixture stirred for 2 h. The progress of the reaction was monitored by TLC and LCMS. After 2 h, the reaction mixture was quenched with sat. Ammonium chloride (10 mL) and extracted with ethyl acetate (2×20 mL). Organic layer was washed with brine solution (10 mL), dried over Na₂SO₄ and concentrated to get crude. Crude compound was purified by normal phase chromatography to afford pure 3-chlorobenzyl ((S)-3-cyclohexyl-1-(((S)-1-hydroxy-5-(methyl(phenethyl)amino)-5-oxopentan-2-yl)amino)-1-oxopropan-2-yl)carbamate (6).

### 3-Chlorobenzyl ((2S)-3-cyclohexyl-1-(((2S)-1,5-dioxo-5-(3-phenylpiperidin-1-yl)pentan-2-yl)amino)-1-oxopropan-2-yl)carbamate A53

To a stirred solution of 3-chlorobenzyl ((2S)-3-cyclohexyl-1-(((2S)-1-hydroxy-5-oxo-5-(3-phenylpiperidin-1-yl)pentan-2-yl)amino)-1-oxopropan-2-yl)carbamate (6) (180 mg, 0.30 mmol) in ethyl acetate (2 mL) was added Dess-Martin periodinane (383 mg, 0.90 mmol) at 0° C. and stirred at RT for 3 h. The progress of the reaction was monitored by TLC and LCMS. Reaction mixture was diluted with ethyl acetate (20 mL) and washed with sat. Hypo solution (3×10 mL), sat. NaHCO₃ solution (3×20 mL). Organic layer was dried over anhydrous Na₂SO₄, filtered and concentrated to get crude compound. Crude was purified by prep-HPLC to afford 3-chlorobenzyl ((2S)-3-cyclohexyl-1-(((2S)-1,5-dioxo-5-(3-phenylpiperidin-1-yl)pentan-2-yl)amino)-1-oxopropan-2-yl)carbamate A53.

TLC system: 70% EtOAc in pet ether; Rf: 0.5

LCMS (ESI): m/z 596.60 (M+H)⁺

### 3-Chlorobenzyl ((2S)-3-cyclohexyl-1-(((2S)-1-(diethoxyphosphoryl)-1-hydroxy-5-oxo-5-(3-phenylpiperidin-1-yl)pentan-2-yl)amino)-1-oxopropan-2-yl)carbamate A106

To a stirred solution of 3-chlorobenzyl ((2S)-3-cyclohexyl-1-(((2S)-1,5-dioxo-5-(3-phenylpiperidin-1-yl)pentan-2-yl)amino)-1-oxopropan-2-yl)carbamate A53 (200 mg, 0.336 mmol) in DCM (2 mL) was added DIPEA (0.17 mL, 1.008 mmol) and diethylphosphite (0.14 mL, 1.008 mmol) at 0° C. and stirred at RT for 16 h. The progress of the reaction was monitored by TLC and LCMS. Reaction mixture was quenched with ice water (10 mL) extracted with DCM (3×20 mL). Organic layer was dried over anhydrous Na₂SO₄, filtered and purified by prep HPLC afforded 3-chlorobenzyl ((2S)-3-cyclohexyl-1-(((2S)-1-(diethoxyphosphoryl)-1-hydroxy-5-oxo-5-(3-phenylpiperidin-1-yl)pentan-2-yl)amino)-1-oxopropan-2-yl)carbamate A106.

TLC system: 70% EtOAc in pet ether; Rf: 0.3

LCMS (ESI): m/z 734.67 (M+H)⁺

### (2S)-2-((S)-2-((((3-chlorobenzyl)oxy)carbonyl)amino)-3-cyclohexylpropanamido)-5-oxo-5-(3-phenylpiperidin-1-yl)pentanoic acid (6)

To a stirred solution of methyl (2S)-2-((S)-2-((((3-chlorobenzyl)oxy)carbonyl)amino)-3-cyclohexylpropanamido)-5-oxo-5-(3-phenylpiperidin-1-yl)pentanoate (5) (500 mg, 0.8 mmol) in THE (5 mL) and water (2 mL), was added lithium hydroxide (67 mg, 1.6 mmol) at RT and stirred at room temperature for 2 h. The progress of the reaction was monitored by TLC and LCMS. After 2 h, the reaction mixture was completely distilled under reduced pressure. Reaction mixture was acidified with 2N HCl solution to adjust pH ˜4, and extracted with ethyl acetate (2×20 mL), dried over sodium sulfate, concentrated under reduced pressure to afford (2S)-2-((S)-2-((((3-chlorobenzyl)oxy)carbonyl)amino)-3-cyclohexylpropanamido)-5-oxo-5-(3-phenylpiperidin-1-yl)pentanoic acid (6).

### 3-chlorobenzyl ((2S)-3-cyclohexyl-1-(((2S)-1-(methoxy(methyl)amino)-1,5-dioxo-5-(3-phenylpiperidin-1-yl)pentan-2-yl)amino)-1-oxopropan-2-yl)carbamate A43

To a stirred solution of (2S)-2-((S)-2-((((3-chlorobenzyl)oxy)carbonyl)amino)-3-cyclohexylpropanamido)-5-oxo-5-(3-phenylpiperidin-1-yl)pentanoic acid (6) (430 mg, 0.7 mmol) in THE (3 mL) and DCM (3 mL) was added EDC.HCl (200 mg, 1.05 mmol), HOBT (135 mg, 1.05 mmol), N-methylmorpholine (0.28 mL, 2.1 mmol) and N,O-dimethylhydroxylamine (137 mg, 1.4 mmol) at 0° C. simultaneously and stirred at room temperature for 16 h. The progress of the reaction was monitored by TLC and LCMS. Reaction mixture was diluted with ice water (10 mL), extracted with ethyl acetate (2×10 mL). The combined organic layer was dried over sodium sulfate, filtered and evaporated under reduced pressure. The crude residue was purified by prep-HPLC to afforded 3-chlorobenzyl ((2S)-3-cyclohexyl-1-(((2S)-1-(methoxy(methyl)amino)-1,5-dioxo-5-(3-phenylpiperidin-1-yl)pentan-2-yl)amino)-1-oxopropan-2-yl)carbamate A43.

TLC system: 10% Methanol in DCM; Rf: 0.6

LCMS (ESI): m/z 655.67 (M+H)⁺

### Synthesis of Compounds A45 and A54

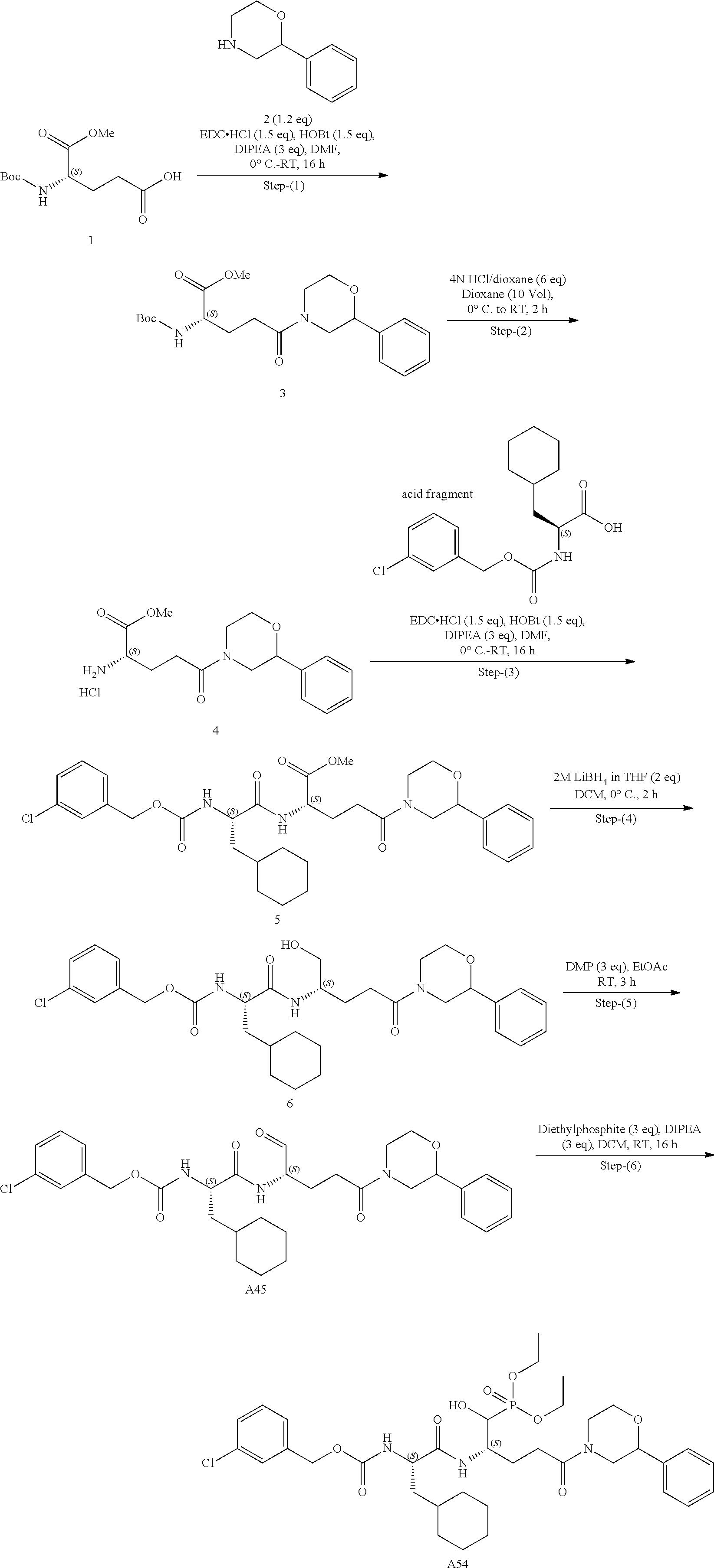

### Methyl (2S)-2-((tert-butoxycarbonyl)amino)-5-oxo-5-(2-phenylmorpholino)pentanoate (3)

To a stirred solution of (S)-4-((tert-butoxycarbonyl)amino)-5-methoxy-5-oxopentanoic acid (1) (2 g, 7.66 mmol) DMF (30 mL) added EDC.HCl (2.1 g, 11.49 mmol), HOBT (1.5 g, 11.49 mmol), Et₃N (3.3 mL, 22.98 mmol) at 0° C. Then reaction mass was stirred for 15 min and then added 2-phenylmorpholine (2) (1.8 g, 9.19 mmol) at 0° C. and stirred at room temperature for 16 h. The progress of the reaction was monitored by TLC and LCMS. After 16 h, the reaction mixture was quenched with ice water (100 mL) and extracted with ethyl acetate (2×60 mL), combined organic layers were washed with brine solution (50 mL), dried over sodium sulfate and evaporated under reduced pressure. The crude residue was purified by normal phase chromatography with eluted 2% methanol in dichloromethane to afford methyl (2S)-2-((tert-butoxycarbonyl)amino)-5-oxo-5-(2-phenylmorpholino)pentanoate (3).

### Methyl (2S)-2-amino-5-oxo-5-(2-phenylmorpholino)pentanoate hydrochloride (4)

To a stirred solution of methyl (2S)-2-((tert-butoxycarbonyl)amino)-5-oxo-5-(2-phenylmorpholino)pentanoate (3) (2.3 g, 5.89 mmol) in 1,4-dioxane (10 mL) was added 4 M HCl in dioxane (10 mL) with drop wise at 0° C. and the reaction mixture was stirred at RT for 2 h. The progress of the reaction was monitored by TLC and LCMS. After 2 h, the reaction mixture was evaporated to dryness under reduced pressure to obtain crude compound, the resulting crude triturated with diethyl ether to afford methyl (2S)-2-amino-5-oxo-5-(2-phenylmorpholino)pentanoate hydrochloride (4).

### Methyl (2S)-2-((S)-2-((((3-chlorobenzyl)oxy)carbonyl)amino)-3-cyclohexylpropanamido)-5-oxo-5-(2-phenylmorpholino)pentanoate (5)

To a stirred solution of (S)-2-((((3-chlorobenzyl)oxy)carbonyl)amino)-3-cyclohexylpropanoic acid (acid fragment) (1.4 g, 3.539 mmol) (synthesis reported in Compd-17 final report) in DMF (30 mL) added EDC.HCl (1.1 g, 6.19 mmol), HOBT (836 mg, 6.19 mmol), DIPEA (2.3 mL, 12.39 mmol) at 0° C. Then reaction mass was stirred for 15 min was methyl (2S)-2-amino-5-oxo-5-(2-phenylmorpholino)pentanoate hydrochloride (4) (1.6 g, 4.95 mmol) at 0° C. stirred at room temperature for 16 h. The progress of the reaction was monitored by TLC and LCMS. After 16 h, the reaction mixture was quenched with ice water (160 mL) and extracted with ethyl acetate (2×80 mL). Combined organic layers were washed with brine solution (80 mL), dried over sodium sulfate and evaporated under reduced pressure to get crude. Crude residue was purified by normal phase chromatography with eluted 70% EtOAc in Pet ether to methyl (2S)-2-((S)-2-((((3-chlorobenzyl)oxy)carbonyl)amino)-3-cyclohexylpropanamido)-5-oxo-5-(2-phenylmorpholino)pentanoate (5).

### 3-Chlorobenzyl ((2S)-3-cyclohexyl-1-(((2S)-1-hydroxy-5-oxo-5-(2-phenylmorpholino)pentan-2-yl)amino)-1-oxopropan-2-yl)carbamate (6)

To a stirred solution of methyl (2S)-2-((S)-2-((((3-chlorobenzyl)oxy)carbonyl)amino)-3-cyclohexylpropanamido)-5-oxo-5-(2-phenylmorpholino)pentanoate (5) (600 mg, 0.95 mmol) in DCM (10 mL) was added 2M LiBH₄ in THE (1.4 mL, 2.85 mmol) at 0° C. Reaction mixture stirred for 2 h at 0° C. The progress of the reaction was monitored by TLC and LCMS. The reaction mixture was quenched with water (20 mL) and extracted with DCM (2×20 mL). The combined organic layer was washed with brine solution (20 mL) and the organic layer was dried over Na₂SO₄ and concentrated to get crude compound. Crude compound was purified by normal phase chromatography to afford 3-chlorobenzyl ((2S)-3-cyclohexyl-1-(((2S)-1-hydroxy-5-oxo-5-(2-phenylmorpholino)pentan-2-yl)amino)-1-oxopropan-2-yl)carbamate (6).

### 3-Chlorobenzyl ((2S)-3-cyclohexyl-1-(((2S)-1,5-dioxo-5-(2-phenylmorpholino)pentan-2-yl)amino)-1-oxopropan-2-yl)carbamate A45

To a stirred solution of 3-chlorobenzyl ((2S)-3-cyclohexyl-1-(((2S)-1-hydroxy-5-oxo-5-(2-phenylmorpholino)pentan-2-yl)amino)-1-oxopropan-2-yl)carbamate (6) (700 mg, 1.17 mmol) was dissolved in ethyl acetate (12 mL) was added Dess-Martin periodinane (1.4 g, 3.505 mmol) at 0° C. and stirred at RT for 3 h. The progress of the reaction was monitored by TLC and LCMS. After 3 h, the reaction mixture was diluted with ethyl acetate (15 mL) and washed with sat. Hypo solution (3×20 mL) and sat. NaHCO₃ solution (3×20 mL). The organic layer was washed with brine solution (20 mL) and dried over Na₂SO₄ and concentrated to get crude compound. Crude was purified by prep HPLC to afford 3-chlorobenzyl ((2S)-3-cyclohexyl-1-(((2S)-1,5-dioxo-5-(2-phenylmorpholino)pentan-2-yl)amino)-1-oxopropan-2-yl)carbamate A45.

TLC system: 5% Methanol in DCM; Rf: 0.4

LCMS (ESI): m/z 598.57 (M+H)⁺

### 3-Chlorobenzyl ((2S)-3-cyclohexyl-1-(((2S)-1-(diethoxyphosphoryl)-1-hydroxy-5-oxo-5-(2-phenylmorpholino)pentan-2-yl)amino)-1-oxopropan-2-yl)carbamate A54

To a stirred solution of 3-chlorobenzyl ((2S)-3-cyclohexyl-1-(((2S)-1,5-dioxo-5-(2-phenylmorpholino)pentan-2-yl)amino)-1-oxopropan-2-yl)carbamate A45 (250 mg, 0.418 mmol) in DCM (3 mL) added DIPEA (0.2 mL, 1.25 mmol) followed by diethyl phosphite (0.2 mL, 1.25 mmol) at 0° C. then reaction mixture stirred at RT for 16 h. The progress of the reaction was monitored by TLC and LCMS. After 16 h, the reaction mixture quenched with water (15 mL) and extracted with DCM (2×20 mL) The combined organic layer was washed with brine solution (10 mL) and dried over Na₂SO₄ and concentrated to get crude residue, which was purified by prep-HPLC afforded 3-chlorobenzyl ((2S)-3-cyclohexyl-1-(((2S)-1-(diethoxyphosphoryl)-1-hydroxy-5-oxo-5-(2-phenylmorpholino)pentan-2-yl)amino)-1-oxopropan-2-yl)carbamate A54.

TLC system: 5% Methanol in DCM; Rf: 0.3

LCMS (ESI): m/z 736.66 (M+H)⁺

### Synthesis of Compounds A68, A51, and A52

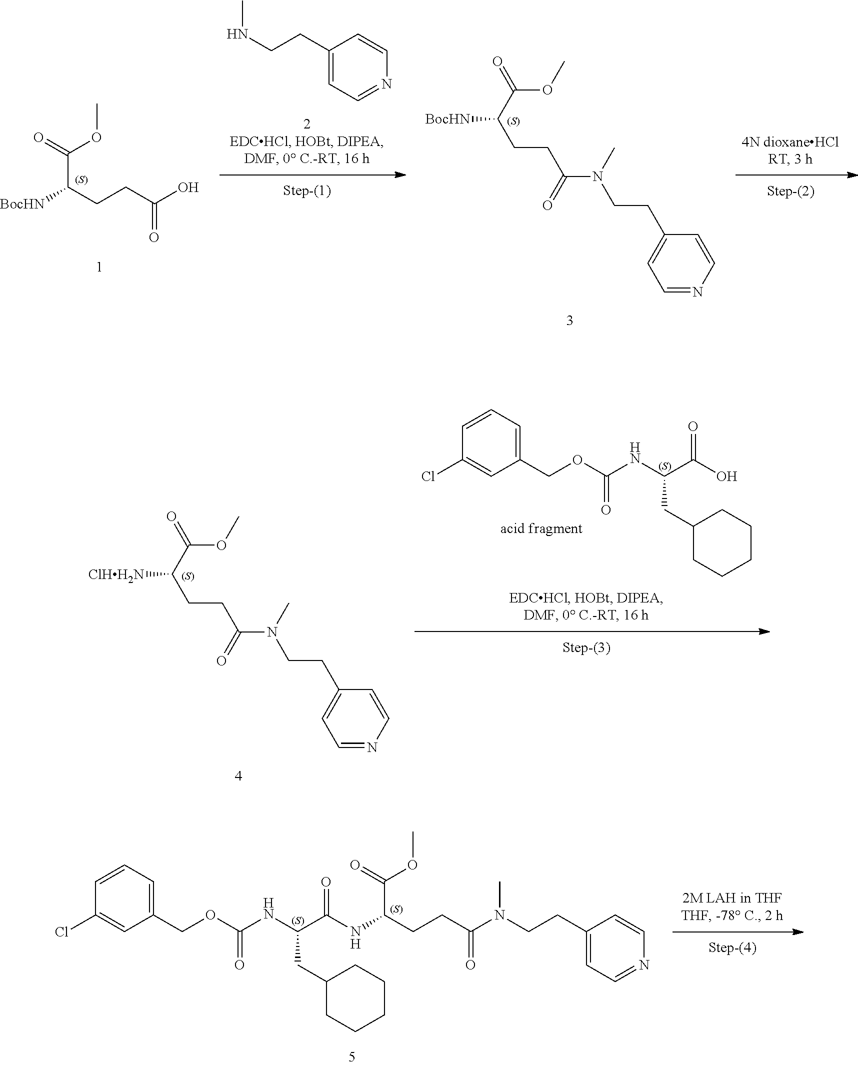

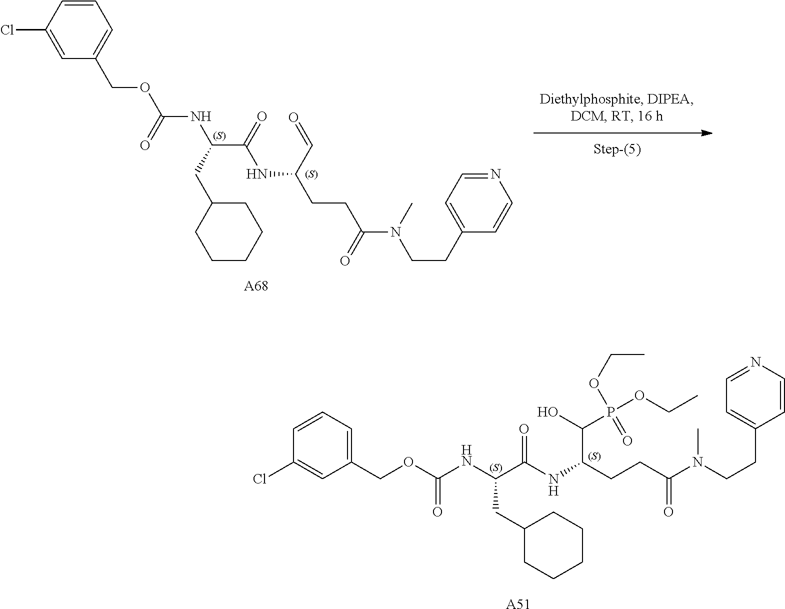

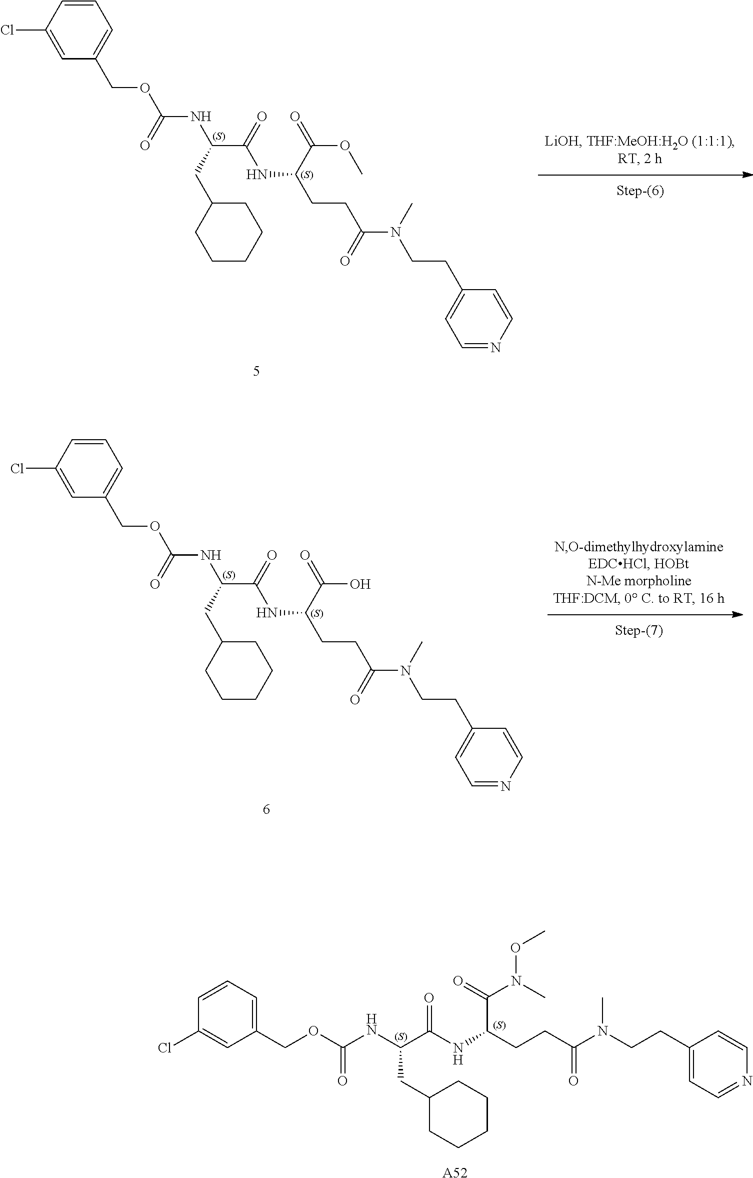

### Methyl N2-(tert-butoxycarbonyl)-N5-methyl-N5-(2-(pyridin-4-yl) ethyl)-L-glutaminate (3)

To a stirred solution of (S)-4-((tert-butoxycarbonyl)amino)-5-methoxy-5-oxopentanoic acid (1) (2 g, 7.66 mmol) in DMF (20 mL) was added EDC.HCl (2.19 g, 11.49 mmol), HOBT (1.56 g, 11.49 mmol), DIPEA (4.1 mL, 22.98 mmol) and N-methyl-2-(pyridin-4-yl)ethan-1-amine (2) (1.18 mL, 8.42 mmol) at 0° C. simultaneously and stirred at room temperature for 16 h. Reaction mixture was diluted with ice water (50 mL), extracted with ethyl acetate (2×40 mL), dried over sodium sulfate and evaporated under reduced pressure. The crude residue was purified by silica gel column by eluting with 4% methanol in dichloromethane to afford methyl N2-(tert-butoxycarbonyl)-N5-methyl-N5-(2-(pyridin-4-yl)ethyl)-L-glutaminate (3).

### Methyl N5-methyl-N5-(2-(pyridin-4-yl) ethyl)-L-glutaminate hydrochloride (4)

To a stirred solution of methyl N2-(tert-butoxycarbonyl)-N5-methyl-N5-(2-(pyridin-4-yl)ethyl)-L-glutaminate (3) (1.4 g, 3.693 mmol) in 1, 4-dioxane (15 mL) was added 4N HCl in dioxane (15 mL) with drop wise at 0° C. Reaction mixture was stirred at RT for 3 h. The progress of the reaction was monitored by TLC. After consumption of starting material, the reaction mixture was evaporated under reduced pressure to obtain crude compound. The resulting crude was triturated with diethyl ether to afford methyl N5-methyl-N5-(2-(pyridin-4-yl)ethyl)-L-glutaminate hydrochloride (4).

### Methyl N2-((S)-2-((((3-chlorobenzyl)oxy)carbonyl)amino)-3-cyclohexylpropanoyl)-N5-methyl-N5-(2-(pyridin-4-yl)ethyl)-L-glutaminate (5)

To a stirred solution of (S)-2-((((3-chlorobenzyl)oxy)carbonyl)amino)-3-cyclohexylpropanoic acid (acid fragment) (1.4 g, 4.32 mmol) in DMF (15 mL) was added EDC.HCl (1.23 g, 6.46 mmol), HOBT (0.86 g, 6.46 mmol), DIPEA (2.3 mL, 12.98 mmol) and methyl N5-methyl-N5-(2-(pyridin-4-yl)ethyl)-L-glutaminate hydrochloride (4) (1.3 g, 4.72 mmol) at 0° C. simultaneously and stirred at room temperature for 16 h. Reaction mixture was diluted with ice water (30 mL), extracted with ethyl acetate (2×40 mL), dried over sodium sulfate and evaporated under reduced pressure. The crude residue was purified by silica gel column by eluting with 3% methanol in dichloromethane to afford methyl N2-((S)-2-((((3-chlorobenzyl) oxy) carbonyl) amino)-3-cyclohexylpropanoyl)-N5-methyl-N5-(2-(pyridin-4-yl) ethyl)-L-glutaminate (5).

### 3-Chlorobenzyl ((S)-3-cyclohexyl-1-(((S)-5-(methyl (2-(pyridin-4-yl) ethyl) amino)-1, 5-dioxopentan-2-yl) amino)-1-oxopropan-2-yl) carbamate A68

To a stirred solution of methyl N2-((S)-2-((((3-chlorobenzyl) oxy) carbonyl) amino)-3-cyclohexylpropanoyl)-N5-methyl-N5-(2-(pyridin-4-yl) ethyl)-L-glutaminate (5) (100 mg, 0.166 mmol) in THE (2 mL) was added 2M LAH in THE (0.07 mL, 0.166 mmol) at −78° C. and the reaction mixture stirred at same temperature for 2 h. The progress of the reaction was monitored by TLC and LCMS. Then reaction mixture was quenched with sat. ammonium chloride solution (5 mL) and extracted with ethyl acetate (2×10 mL). Organic layer was washed with brine solution (15 mL), dried over Na₂SO₄ and concentrated to afford 3-chlorobenzyl ((S)-3-cyclohexyl-1-(((S)-5-(methyl (2-(pyridin-4-yl) ethyl) amino)-1, 5-dioxopentan-2-yl) amino)-1-oxopropan-2-yl) carbamate A68.

### 3-Chlorobenzyl ((2S)-3-cyclohexyl-1-(((2S)-1-(diethoxyphosphoryl)-1-hydroxy-5-(methyl (2-(pyridin-4-yl) ethyl) amino)-5-oxopentan-2-yl) amino)-1-oxopropan-2-yl) carbamate A51

To a stirred solution of 3-chlorobenzyl ((S)-3-cyclohexyl-1-(((S)-5-(methyl (2-(pyridin-4-yl) ethyl) amino)-1, 5-dioxopentan-2-yl) amino)-1-oxopropan-2-yl) carbamate A68 (69 mg, 0.121 mmol) in DCM (2 mL) was added DIPEA (0.06 mL, 0.363 mmol) followed by addition of diethylphosphite (0.05 mL, 0.363 mmol) and the reaction mixture stirred at RT for 16 h. The progress of the reaction was monitored by TLC and LCMS. After 16 h, the reaction mixture quenched with ammonium chloride (5 mL) and extracted with DCM (2×10 mL). Combined organic layer was dried over anhydrous Na₂SO₄, and evaporated to afford crude residue. It was purified prep HPLC to afford 3-chlorobenzyl ((2S)-3-cyclohexyl-1-(((2S)-1-(diethoxyphosphoryl)-1-hydroxy-5-(methyl(2-(pyridin-4-yl)ethyl)amino)-5-oxopentan-2-yl)amino)-1-oxopropan-2-yl)carbamate A51.

TLC system: 5% Methanol in dichloromethane; Rf: 0.4

LCMS (ESI): m/z 709.66 (M+H)⁺

### N2-((S)-2-((((3-chlorobenzyl)oxy)carbonyl)amino)-3-cyclohexylpropanoyl)-N5-methyl-N5-(2-(pyridin-4-yl)ethyl)-L-glutamine (6)

To a stirred solution of methyl N2-((S)-2-((((3-chlorobenzyl) oxy) carbonyl) amino)-3-cyclohexylpropanoyl)-N5-methyl-N5-(2-(pyridin-4-yl) ethyl)-L-glutaminate (5) (280 mg, 0.466 mmol) in mixture of solvents THE (3 mL), methanol (3 mL) and water (3 mL) was added lithium hydroxide (30 mg, 1.162 mmol) at 0° C. and stirred at room temperature for 2 h. The progress of the reaction was monitored by TLC and LCMS. Reaction mixture completely distilled under reduced pressure, crude compound was acidified with 2N HCl solution up to pH ˜3 and extracted with ethyl acetate (2×15 mL), dried over sodium sulfate, concentrated under reduced pressure to afford N2-((S)-2-((((3-chlorobenzyl)oxy)carbonyl)amino)-3-cyclohexylpropanoyl)-N5-methyl-N5-(2-(pyridin-4-yl)ethyl)-L-glutamine (6). 3-Chlorobenzyl ((S)-3-cyclohexyl-1-(((S)-1-(methoxy (methyl) amino)-5-(methyl (2-(pyridin-4-yl) ethyl) amino)-1, 5-dioxopentan-2-yl) amino)-1-oxopropan-2-yl)carbamate A52

To a stirred solution of N2-((S)-2-((((3-chlorobenzyl)oxy)carbonyl)amino)-3-cyclohexylpropanoyl)-N5-methyl-N5-(2-(pyridin-4-yl)ethyl)-L-glutamine (6) (230 mg, 0.391 mmol) in THE (5 mL) and DCM (5 mL) was added EDC.HCl (112 mg, 0.581 mmol), HOBT (79 mg, 0.581 mmol), N-methylmorpholine (0.17 mL, 1.173 mmol) and N,O-dimethylhydroxylamine (45 mg, 0.472 mmol) at 0° C. simultaneously and stirred at room temperature for 16 h. The progress of the reaction was monitored by TLC and LCMS. Then the reaction mixture was quenched with ice water (20 mL) and extracted with ethyl acetate (2×20 mL). The combined organic layer was dried over sodium sulfate, filtered and evaporated under reduced pressure. The crude residue was purified by prep-HPLC to afforded 3-chlorobenzyl ((S)-3-cyclohexyl-1-(((S)-1-(methoxy(methyl)amino)-5-(methyl(2-(pyridin-4-yl)ethyl)amino)-1,5-dioxopentan-2-yl)amino)-1-oxopropan-2-yl)carbamate A52.

TLC system: 10% Methanol in DCM; Rf: 0.5

LCMS (ESI): m/z 630.50 (M+H)⁺

### Synthesis of Compounds A107 and A55

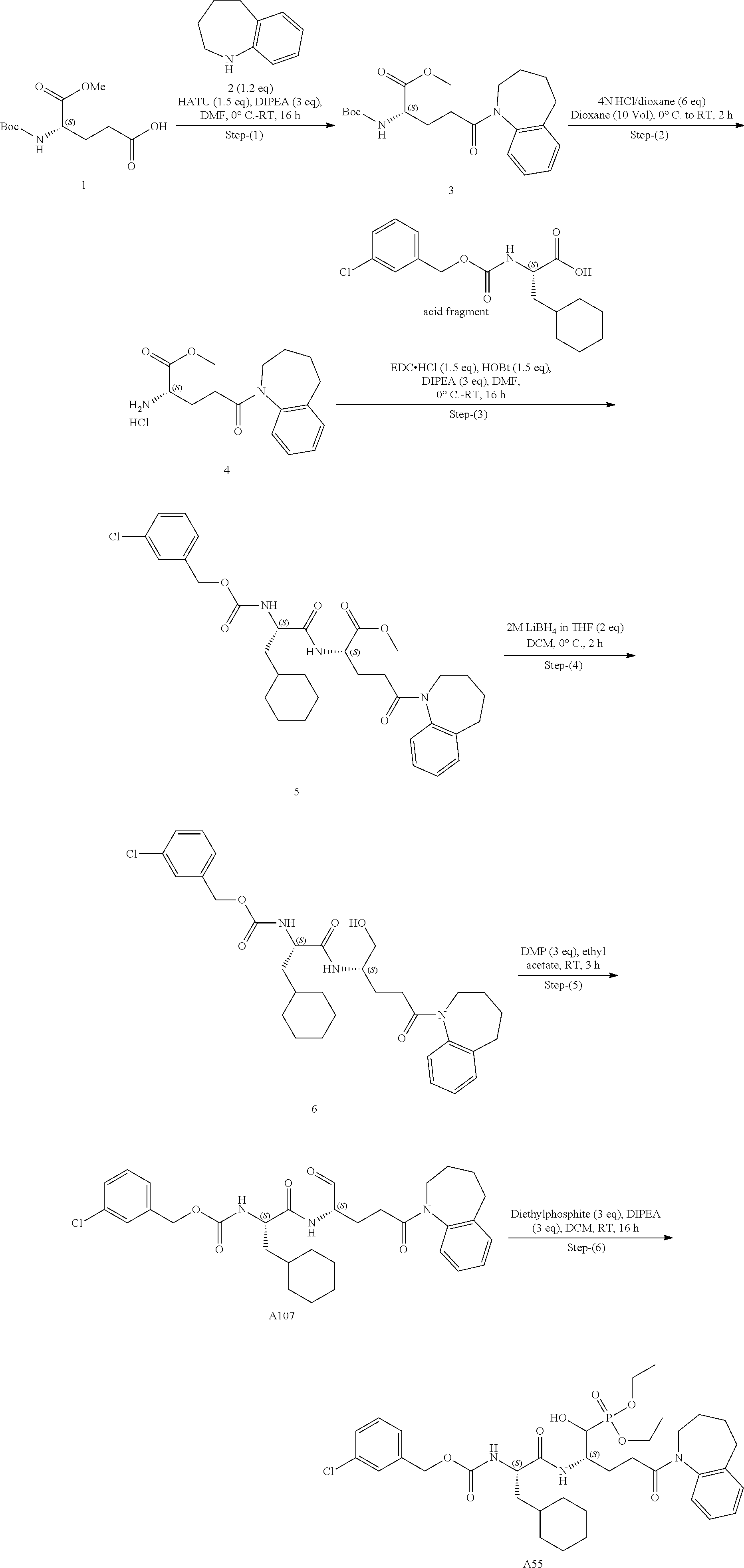

### Methyl (S)-2-((tert-butoxycarbonyl)amino)-5-oxo-5-(2,3,4,5-tetrahydro-1H-benzo[b]azepin-1-yl)pentanoate (3)

To a stirred solution of (S)-4-((tert-butoxycarbonyl)amino)-5-methoxy-5-oxopentanoic acid (1) (2 g, 7.66 mmol) DMF (20 mL) added HATU (5.8 g, 15.32 mmol), DIPEA (5.6 mL, 30.65 mmol) at 0° C. Then reaction mass was stirred for 15 min and then added 2,3,4,5-tetrahydro-1H-benzo[b]azepine (2) (1.3 g, 9.19 mmol) at 0° C. and stirred at room temperature for 16 h. The progress of the reaction was monitored by TLC and LCMS. Reaction mixture was quenched with ice water (100 mL) and extracted with ethyl acetate (2×60 mL), combined organic layers were washed with brine solution (50 mL), dried over sodium sulfate and evaporated under reduced pressure. The crude residue was purified by normal phase chromatography with eluted 4% methanol in dichloromethane to afford methyl (S)-2-((tert-butoxycarbonyl)amino)-5-oxo-5-(2,3,4,5-tetrahydro-1H-benzo[b]azepin-1-yl)pentanoate (3).

### Methyl (S)-2-amino-5-oxo-5-(2,3,4,5-tetrahydro-1H-benzo[b]azepin-1-yl)pentanoate hydrochloride (4)

To a stirred solution of methyl (S)-2-((tert-butoxycarbonyl)amino)-5-oxo-5-(2,3,4,5-tetrahydro-1H-benzo[b]azepin-1-yl)pentanoate (3) (520 mg, 1.33 mmol) in 1,4-dioxane (5 mL) was added 4 M HCl in dioxane (5 mL) with drop wise at 0° C. and the reaction mixture was stirred at RT for 2 h. The progress of the reaction was monitored by TLC. The reaction mixture was evaporated to dryness under reduced pressure to obtain crude compound, the resulting crude triturated with diethyl ether to afford methyl (S)-2-amino-5-oxo-5-(2,3,4,5-tetrahydro-1H-benzo[b]azepin-1-yl)pentanoate hydrochloride (4).

### Methyl (S)-2-((S)-2-((((3-chlorobenzyl)oxy)carbonyl)amino)-3-cyclohexylpropanamido)-5-oxo-5-(2,3,4,5-tetrahydro-1H-benzo[b]azepin-1-yl)pentanoate (5)

To a stirred solution of (S)-2-((((3-chlorobenzyl)oxy)carbonyl)amino)-3-cyclohexylpropanoic acid (acid fragment) (1.2 g, 3.53 mmol) (synthesis reported in Compd-17 final report) in DMF (30 mL) added EDC.HCl (1 g, 5.31 mmol), HOBT (716 mg, 5.31 mmol), DIPEA (2 mL, 10.61 mmol) at 0° C. Then reaction mass was stirred for 15 min, then added methyl (S)-2-amino-5-oxo-5-(2,3,4,5-tetrahydro-1H-benzo[b]azepin-1-yl)pentanoate (4) (1.38 g, 4.24 mmol) at 0° C. stirred at room temperature for 16 h. The progress of the reaction was monitored by TLC and LCMS. After 16 h, the reaction mixture was quenched with ice water (100 mL) and extracted with ethyl acetate (2×80 mL). Combined organic layers were washed with brine solution (80 mL), dried over sodium sulfate and evaporated under reduced pressure to get crude. Crude residue was purified by normal phase chromatography with eluted 60% EtOAc in Pet ether to afford methyl (S)-2-((S)-2-((((3-chlorobenzyl)oxy)carbonyl)amino)-3-cyclohexylpropanamido)-5-oxo-5-(2,3,4,5-tetrahydro-1H-benzo[b]azepin-1-yl)pentanoate (5).

### 3-Chlorobenzyl ((S)-3-cyclohexyl-1-(((S)-1-hydroxy-5-oxo-5-(2,3,4,5-tetrahydro-1H-benzo[b]azepin-1-yl)pentan-2-yl)amino)-1-oxopropan-2-yl)carbamate (6)

At 0° C., to a stirred solution of methyl (S)-2-((S)-2-((((3-chlorobenzyl)oxy)carbonyl)amino)-3-cyclohexylpropanamido)-5-oxo-5-(2,3,4,5-tetrahydro-1H-benzo[b]azepin-1-yl)pentanoate (5) (800 mg, 1.37 mmol) in DCM (5 mL) was added 2M LiBH₄ in THE (2 mL, 4.11 mmol) and stirred for 2 h at 0° C. The progress of the reaction was monitored by TLC and LCMS. The reaction mixture was quenched with water (20 mL) and extracted with DCM (2×20 mL). The combined organic layer was washed with brine solution (20 mL) and the organic layer was dried over Na₂SO₄ and concentrated to get crude compound. Crude compound was purified by normal phase chromatography to afford 3-chlorobenzyl ((S)-3-cyclohexyl-1-(((S)-1-hydroxy-5-oxo-5-(2,3,4,5-tetrahydro-1H-benzo[b]azepin-1-yl)pentan-2-yl)amino)-1-oxopropan-2-yl)carbamate (6).

### 3-Chlorobenzyl ((S)-3-cyclohexyl-1-(((S)-1,5-dioxo-5-(2,3,4,5-tetrahydro-1H-benzo[b]azepin-1-yl)pentan-2-yl)amino)-1-oxopropan-2-yl)carbamate A107

To a stirred solution of 3-chlorobenzyl ((S)-3-cyclohexyl-1-(((S)-1-hydroxy-5-oxo-5-(2,3,4,5-tetrahydro-1H-benzo[b]azepin-1-yl)pentan-2-yl)amino)-1-oxopropan-2-yl)carbamate (6) (500 mg, 0.85 mmol) in ethyl acetate (4 mL) was added Dess-Martin periodinane (1 g, 2.57 mmol) at 0° C. and stirred at RT for 3 h. The progress of the reaction was monitored by TLC and LCMS. After 3 h, the reaction mixture was diluted with ethyl acetate (20 mL) and washed with sat. Hypo solution (3×15 mL) and sat. NaHCO₃ solution (3×15 mL). The organic layer was washed with brine solution (25 mL) and dried over Na₂SO₄ and concentrated to get crude compound. Crude was purified by prep HPLC to afford 3-chlorobenzyl ((S)-3-cyclohexyl-1-(((S)-1,5-dioxo-5-(2,3,4,5-tetrahydro-1H-benzo[b]azepin-1-yl)pentan-2-yl)amino)-1-oxopropan-2-yl)carbamate A107.

TLC system: 10% Methanol in DCM; Rf: 0.4

LCMS (ESI): m/z 582.52 (M+H)⁺

### 3-Chlorobenzyl ((2S)-3-cyclohexyl-1-(((2S)-1-(diethoxyphosphoryl)-1-hydroxy-5-oxo-5-(2,3,4,5-tetrahydro-1H-benzo[b]azepin-1-yl)pentan-2-yl)amino)-1-oxopropan-2-yl)carbamate A55

To a stirred solution of 3-chlorobenzyl ((S)-3-cyclohexyl-1-(((S)-1,5-dioxo-5-(2,3,4,5-tetrahydro-1H-benzo[b]azepin-1-yl)pentan-2-yl)amino)-1-oxopropan-2-yl)carbamate A107 (400 mg, 0.68 mmol) in DCM (4 mL) was added DIPEA (0.38 mL, 2.06 mmol) followed by diethylphosphite (0.4 mL, 2.06 mmol) at 0° C. then reaction mixture stirred at RT for 16 h. The progress of the reaction was monitored by TLC and LCMS. After 16 h, the reaction mixture quenched with water (120 mL) and extracted with DCM (2×20 mL). The combined organic layer was washed with brine solution (10 mL) and dried over Na₂SO₄ and concentrated to get crude. Crude was purified by prep-HPLC afforded 3-chlorobenzyl ((2S)-3-cyclohexyl-1-(((2S)-1-(diethoxyphosphoryl)-1-hydroxy-5-oxo-5-(2,3,4,5-tetrahydro-1H-benzo[b]azepin-1-yl)pentan-2-yl)amino)-1-oxopropan-2-yl)carbamate A55.

TLC system: 5% Methanol in DCM; Rf: 0.2

LCMS (ESI): m/z 720.67 (M+H)⁺

### Synthesis of A57 and A113

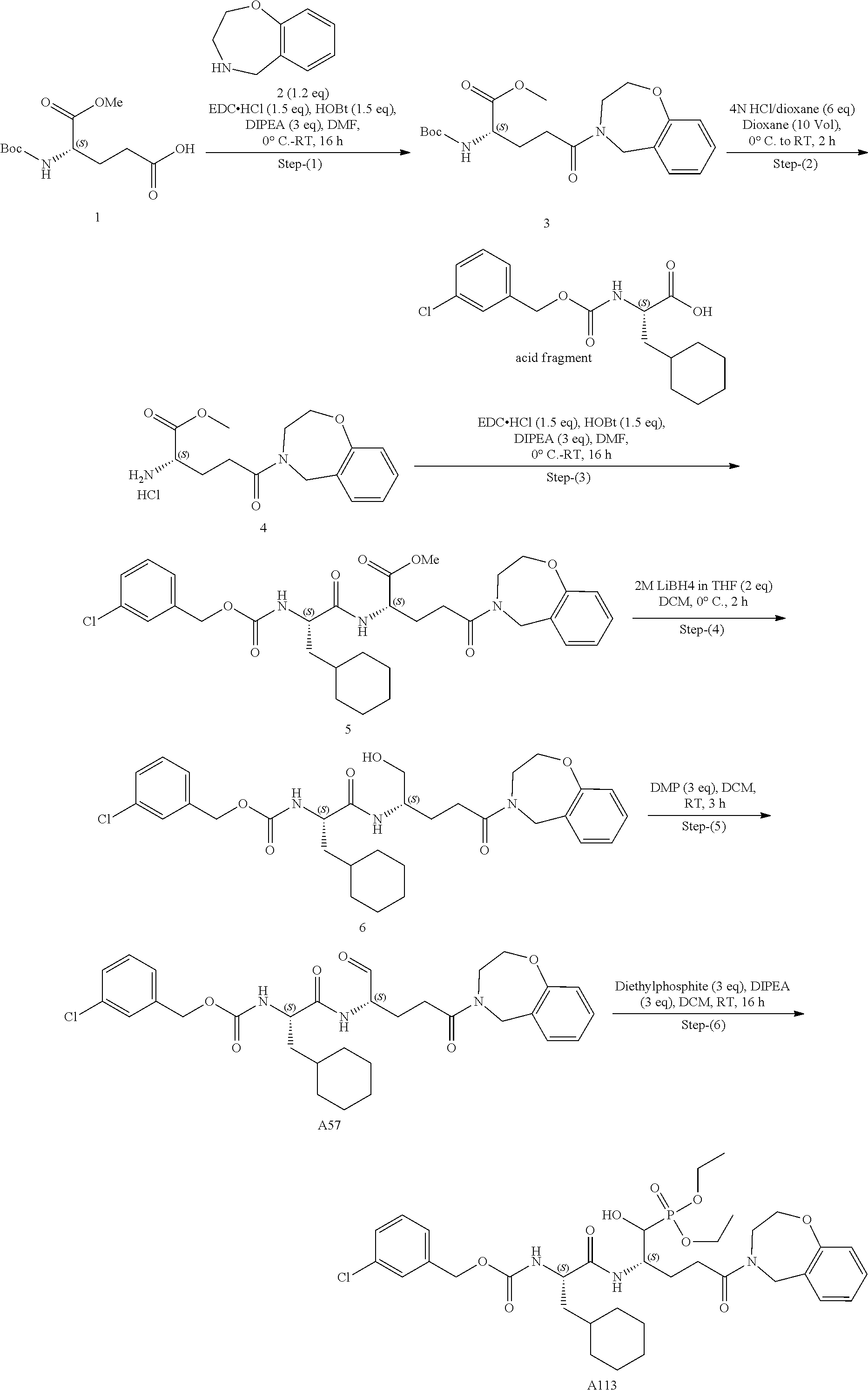

### Methyl (S)-2-((tert-butoxycarbonyl)amino)-5-(2,3-dihydrobenzo[f][1,4]oxazepin-4(5H)-yl)-5-oxopentanoate (3)

At 0° C., to a stirred solution of (S)-4-((tert-butoxycarbonyl)amino)-5-methoxy-5-oxopentanoic acid (1) (2.3 g, 8.821 mmol) DMF (30 mL) was added EDC.HCl (2.5 g, 13.231 mmol), HOBT (1.78 g, 13.231 mmol), DIPEA (4.6 mL, 26.463 mmol) and the reaction mass was stirred for 15 min. After 15 min, added 2,3,4,5-tetrahydrobenzo[f][1,4]oxazepine (2) (1.87 g, 10.134 mmol) at 0° C. and stirred at room temperature for 16 h. The progress of the reaction was monitored by TLC and LCMS. After 16 h, the reaction mixture was quenched with ice water (100 mL) and extracted with ethyl acetate (2×60 mL), combined organic layers were washed with brine solution (50 mL), dried over sodium sulfate and evaporated under reduced pressure. The crude residue was purified by normal phase chromatography with eluted 2% methanol in dichloromethane to afford methyl (S)-2-((tert-butoxycarbonyl)amino)-5-(2,3-dihydrobenzo[f][1,4]oxazepin-4(5H)-yl)-5-oxopentanoate (3).

### Methyl (S)-2-amino-5-(2,3-dihydrobenzo[f][1,4]oxazepin-4(5H)-yl)-5-oxopentanoate hydrochloride (4)

To a stirred solution of methyl (S)-2-((tert-butoxycarbonyl)amino)-5-(2,3-dihydrobenzo[f][1,4]oxazepin-4(5H)-yl)-5-oxopentanoate (3) (2.7 g, 6.887 mmol) in 1,4-dioxane (27 mL) was added 4 M HCl in dioxane (11 mL) with drop wise at 0° C. and the reaction mixture was stirred at RT for 2 h. The progress of the reaction was monitored by TLC and LCMS. After 2 h, the reaction mixture was evaporated to dryness under reduced pressure to obtain crude compound, the resulting crude triturated with diethyl ether to afford methyl (S)-2-amino-5-(2,3-dihydrobenzo[f][1,4]oxazepin-4(5H)-yl)-5-oxopentanoate hydrochloride (4).

### Methyl (S)-2-((S)-2-((((3-chlorobenzyl)oxy)carbonyl)amino)-3-cyclohexylpropanamido)-5-(2,3-dihydrobenzo[f][1,4]oxazepin-4(5H)-yl)-5-oxopentanoate (5)

At 0° C., to a stirred solution of (S)-2-((((3-chlorobenzyl)oxy)carbonyl)amino)-3-cyclohexylpropanoic acid (acid fragment) (1.2 g, 3.539 mmol) (synthesis reported in Compd-17 final report) in DMF (30 mL) added EDC.HCl (1.01 g, 5.308 mmol), HOBT (710 mg, 5.308 mmol), DIPEA (1.8 mL, 10.617 mmol) and the reaction mass was stirred for 15 min. After 15 min, added methyl (S)-2-amino-5-(2,3-dihydrobenzo[f][1,4]oxazepin-4(5H)-yl)-5-oxopentanoate hydrochloride (4) (1.38 g, 4.247 mmol) and stirred at room temperature for 16 h. The progress of the reaction was monitored by TLC and LCMS. After 16 h, the reaction mixture was quenched with ice water (150 mL) and extracted with ethyl acetate (2×80 mL). Combined organic layers were washed with brine solution (80 mL), the organic layer was dried over sodium sulfate and evaporated under reduced pressure to get crude. Crude residue was purified by normal phase chromatography with eluted 50% EtOAc in Pet ether to afford methyl (S)-2-((S)-2-((((3-chlorobenzyl)oxy)carbonyl)amino)-3-cyclohexylpropanamido)-5-(2,3-dihydrobenzo[f][1,4]oxazepin-4(5H)-yl)-5-oxopentanoate (5).

### 3-Chlorobenzyl ((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)carbamate (6)

At 0° C., to a stirred solution of methyl (S)-2-((S)-2-((((3-chlorobenzyl)oxy)carbonyl)amino)-3-cyclohexylpropanamido)-5-(2,3-dihydrobenzo[f][1,4]oxazepin-4(5H)-yl)-5-oxopentanoate (5) (500 mg, 0.815 mmol) in DCM (5 mL) was added 2M LiBH₄ in THE (0.8 mL, 1.63 mmol) and the reaction mixture was stirred for 2 h. The progress of the reaction was monitored by TLC and LCMS. The reaction mixture was quenched with water (20 mL) and extracted with DCM (2×20 mL). The combined organic layer was washed with brine solution (20 mL) and the organic layer was dried over Na₂SO₄ and concentrated to get crude compound. Crude compound was purified by normal phase chromatography to afford 3-chlorobenzyl ((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)carbamate (6).

### 3-Chlorobenzyl ((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)carbamate A57

At 0° C., to a stirred solution of 3-chlorobenzyl ((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)carbamate (6) (300 mg, 0.516 mmol) in ethyl acetate (4 mL) was added Dess-Martin periodinane (749 mg, 1.549 mmol) and stirred at RT for 3 h. The progress of the reaction was monitored by TLC and LCMS. After 3 h, the reaction mixture was diluted with ethyl acetate (15 mL) and washed with sat. Hypo solution (3×20 mL) and sat. NaHCO₃ solution (3×20 mL). The organic layer was washed with brine solution (20 mL) and dried over Na₂SO₄ and concentrated to get crude compound. Crude was purified by prep HPLC to afford 3-chlorobenzyl ((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)carbamate A57.

TLC system: 10% Methanol in DCM; Rf: 0.4

LCMS (ESI): m/z 584.37 (M+H)⁺

### Synthesis of A108 and A58

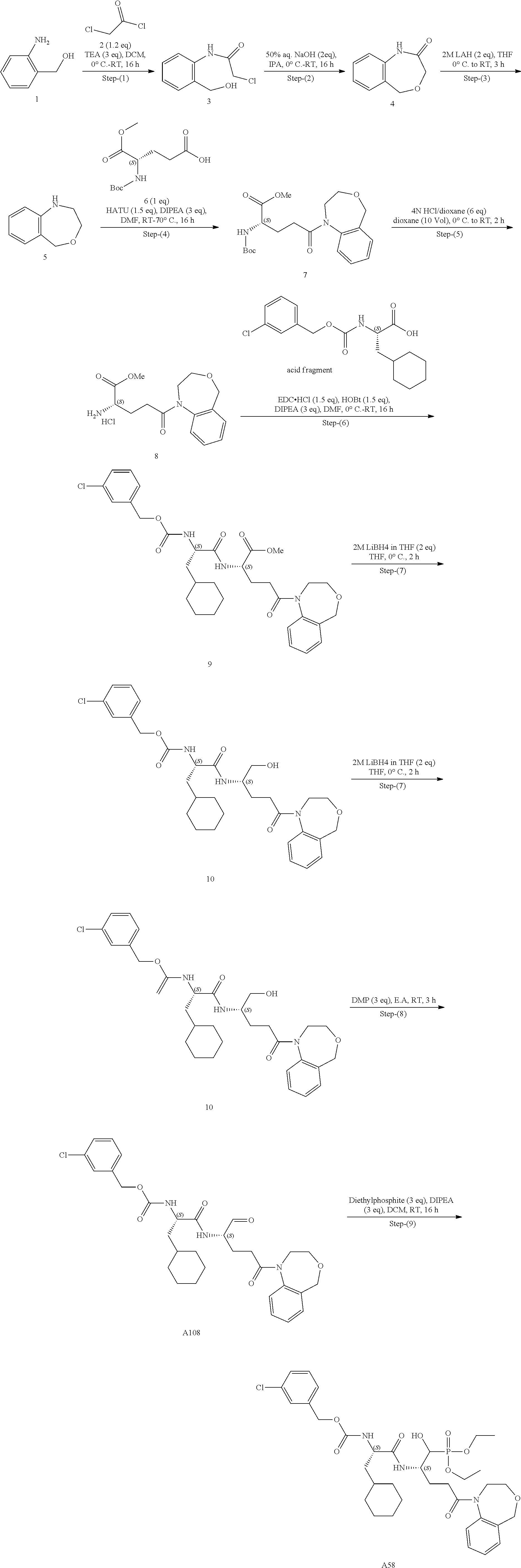

### 2-chloro-N-(2-(hydroxymethyl)phenyl)acetamide (3)

At 0° C., to a stirred solution of (2-aminophenyl) methanol (1) (10 g, 40.65 mmol) in DCM (50 mL) were added TEA (17.5 mL, 121.951 mmol) and stirred for 10 min. After 10 min, added 2-chloroacetyl chloride (2) (4 mL, 49.951 mmol) and stirred at RT for 16 h. The progress of the reaction was monitored by TLC and LCMS. After 16 h, the reaction mixture was diluted with ice water (50 mL), extracted with DCM (2×100 mL). The combined organic layer was dried over sodium sulfate, filtered and evaporated under reduced pressure. The crude residue was purified by combi-flash compound eluted at 20% ethyl acetate in pet ether to afford 2-chloro-N-(2-(hydroxymethyl)phenyl)acetamide (3).

### 1,5-dihydrobenzo[e][1,4]oxazepin-2(3H)-one (4)

To a stirred solution of 2-chloro-N-(2-(hydroxymethyl)phenyl)acetamide (3) (6.5 g, 32.663 mmol) in IPA (70 mL) was added 10% NaOH solution (5.2 mL, 65.32 mmol) at 0° C. and the reaction mixture was stirred at RT for 16 h. The progress of the reaction was monitored by TLC and LCMS. After 16 h, the reaction mixture was diluted with ice water (100 mL), extracted with ethyl acetate (2×100 mL). The combined organic layer was dried over sodium sulfate, filtered and evaporated under reduced pressure. The crude residue was purified by combi-flash compound eluted at 20% ethyl acetate in pet ether to afford 1,5-dihydrobenzo[e][1,4]oxazepin-2(3H)-one (4).

### 1,2,3,5-tetrahydrobenzo[e][1,4]oxazepine (5)

At 0° C., to a stirred solution of 1,5-dihydrobenzo[e][1,4]oxazepin-2(3H)-one (4) (2.2 g, 13.496 mmol) in THE (20 mL) was added 2M LAH solution (13.4 mL, 26.993 mmol) drop-wise and the reaction mixture was stirred at RT for 3 h. The progress of the reaction was monitored by TLC and LCMS. After 3 h, the reaction mixture was diluted with ice water (100 mL), extracted with ethyl acetate (2×100 mL). The combined organic layer was dried over sodium sulfate, filtered and evaporated under reduced pressure. The crude residue was purified by combi-flash compound eluted at 10% Ethyl acetate in pet ether to afforded 1,2,3,5-tetrahydrobenzo[e][1,4]oxazepine (5).

### Methyl (S)-2-((tert-butoxycarbonyl)amino)-5-(2,3-dihydrobenzo[e][1,4]oxazepin-1(5H)-yl)-5-oxopentanoate (7)

To a stirred solution of (S)-4-((tert-butoxycarbonyl)amino)-5-methoxy-5-oxopentanoic acid (6) (2 g, 3.831 mmol) in DMF (10 mL) were added HATU (2.95 g, 7.662 mmol), DIPEA (2.1 mL, 28.7356 mmol) and 1,2,3,5-tetrahydrobenzo[e][1,4]oxazepine (5) (685 mg, 4.597 mmol) at 0° C. and the reaction mixture was stirred at RT for 16 h. The progress of the reaction was monitored by TLC and LCMS. After 16 h, the reaction mixture was diluted with ice water (50 mL), extracted with ethyl acetate (2×50 mL). The combined organic layer was dried over sodium sulfate, filtered and evaporated under reduced pressure. The crude residue was purified by combi-flash compound eluted at 40% Ethyl acetate in pet ether to afford methyl (S)-2-((tert-butoxycarbonyl)amino)-5-(2,3-dihydrobenzo[e][1,4]oxazepin-1(5H)-yl)-5-oxopentanoate (7).

### Methyl (S)-2-amino-5-(2,3-dihydrobenzo[e][1,4]oxazepin-1(5H)-yl)-5-oxopentanoate hydrochloride (8)

To a stirred solution of methyl (S)-2-((tert-butoxycarbonyl)amino)-5-(2,3-dihydrobenzo[e][1,4]oxazepin-1(5H)-yl)-5-oxopentanoate (7) (1.3 g, 3.3165 mmol) in 1,4-dioxane (10 mL) was added 4N HCl in dioxane (10 mL) with drop wise at 0° C. and the reaction mixture was stirred at RT for 2 h. The progress of the reaction was monitored by TLC and LCMS. After consumption of starting material, the reaction mixture was evaporated under reduced pressure to obtained crude compound, the resulting crude triturated with diethyl ether to afford methyl (S)-2-amino-5-(2,3-dihydrobenzo[e][1,4]oxazepin-1(5H)-yl)-5-oxopentanoate hydrochloride (8).

### Methyl (S)-2-((S)-2-((((3-chlorobenzyl)oxy)carbonyl)amino)-3-cyclohexylpropanamido)-5-(2,3-dihydrobenzo[e][1,4]oxazepin-1(5H)-yl)-5-oxopentanoate (9)

At 0° C., to a stirred solution of (S)-2-((((3-chlorobenzyl)oxy)carbonyl)amino)-3-cyclohexylpropanoic acid (acid fragment) (1.2 g, 3.539 mmol) in DMF (20 mL) was added EDC.HCl (1.01 g, 5.309 mmol), HOBT (0.716 g, 5.309 mmol), DIPEA (1.9 mL, 10.619 mmol) and methyl (S)-2-amino-5-(2,3-dihydrobenzo[e][1,4]oxazepin-1(5H)-yl)-5-oxopentanoate hydrogen chloride (8) (1.24 g, 4.247 mmol) sequentially and stirred at room temperature for 16 h. The progress of the reaction was monitored by TLC and LCMS. After 16 h, reaction mixture was diluted with ice water (30 mL), extracted with ethyl acetate (2×50 mL). The combined organic layer was dried over sodium sulfate, filtered and evaporated under reduced pressure. The crude residue was purified by combi-flash, compound eluted at 60% Ethyl acetate in pet ether to afford methyl (S)-2-((S)-2-((((3-chlorobenzyl)oxy)carbonyl)amino)-3-cyclohexylpropanamido)-5-(2,3-dihydrobenzo[e][1,4]oxazepin-1(5H)-yl)-5-oxopentanoate (9).

### 3-Chlorobenzyl ((S)-3-cyclohexyl-1-(((S)-5-(2,3-dihydrobenzo[e][1,4]oxazepin-1(5H)-yl)-1-hydroxy-5-oxopentan-2-yl)amino)-1-oxopropan-2-yl)carbamate (10)

At 0° C., to a stirred solution of methyl (S)-2-((S)-2-((((3-chlorobenzyl)oxy)carbonyl)amino)-3-cyclohexylpropanamido)-5-(2,3-dihydrobenzo[e][1,4]oxazepin-1(5H)-yl)-5-oxopentanoate (9) (1.3 g, 2.1207 mmol) in THE (15 mL) was added 2M LiBH₄ in THE (2.1 mL, 4.241 mmol) and the reaction mixture stirred at same temperature for 2 h. The progress of the reaction was monitored by TLC and LCMS. Then reaction mixture was quenched with water (10 mL) and extracted with ethyl acetate (2×30 mL). Organic layer was washed with brine solution (10 mL), dried over Na₂SO₄ and concentrated to get crude compound. It was purified combi-flash, compound eluted at 80% Ethyl acetate in pet ether to afford 3-chlorobenzyl ((S)-3-cyclohexyl-1-(((S)-5-(2,3-dihydrobenzo[e][1,4]oxazepin-1(5H)-yl)-1-hydroxy-5-oxopentan-2-yl)amino)-1-oxopropan-2-yl)carbamate (10).

### 3-Chlorobenzyl ((S)-3-cyclohexyl-1-(((S)-5-(2,3-dihydrobenzo[e][1,4]oxazepin-1(5H)-yl)-1,5-dioxopentan-2-yl)amino)-1-oxopropan-2-yl)carbamate A108

To a stirred solution of 3-chlorobenzyl ((S)-3-cyclohexyl-1-(((S)-5-(2,3-dihydrobenzo[e][1,4]oxazepin-1(5H)-yl)-1-hydroxy-5-oxopentan-2-yl)amino)-1-oxopropan-2-yl)carbamate (10) (200 mg, 0.341 mmol) in ethyl acetate (10 mL) was added Dess-Martin periodinane (290 mg, 0.682 mmol) at 0° C. and stirred at RT for 3 h. Reaction mixture was diluted with DCM (10 mL) and washed with sat. Hypo solution (3×10 mL), sat. NaHCO₃ solution (3×20 mL). Organic layer was dried over anhydrous Na₂SO₄, filtered and concentrated to afford 3-chlorobenzyl ((S)-3-cyclohexyl-1-(((S)-5-(2,3-dihydrobenzo[e][1,4]oxazepin-1(5H)-yl)-1,5-dioxopentan-2-yl)amino)-1-oxopropan-2-yl)carbamate crude compound A108.

### 3-Chlorobenzyl ((2S)-3-cyclohexyl-1-(((2S)-1-(diethoxyphosphoryl)-5-(2,3-dihydrobenzo[e][1,4]oxazepin-1(5H)-yl)-1-hydroxy-5-oxopentan-2-yl)amino)-1-oxopropan-2-yl)carbamate A58

To a stirred solution of 3-chlorobenzyl ((S)-3-cyclohexyl-1-(((S)-5-(2,3-dihydrobenzo[e][1,4]oxazepin-1(5H)-yl)-1,5-dioxopentan-2-yl)amino)-1-oxopropan-2-yl)carbamate crude compound A108 (200 mg, 0.342 mmol) in DCM (10 mL) was added DIPEA (0.2 mL, 1.027 mmol) followed by added diethylphosphite (0.1 mL, 0.684 mmol) and the reaction mixture stirred at RT for 16 h. The progress of the reaction was monitored by TLC and LCMS. After 16 h, the reaction mixture quenched with ammonium chloride solution (15 mL) and extracted with DCM (2×20 mL). Combined organic layer was dried over anhydrous Na₂SO₄, and evaporated to afford crude residue. The crude residue was purified by prep HPLC to afford 3-chlorobenzyl ((2S)-3-cyclohexyl-1-(((2S)-1-(diethoxyphosphoryl)-5-(2,3-dihydrobenzo[e][1,4]oxazepin-1(5H)-yl)-1-hydroxy-5-oxopentan-2-yl)amino)-1-oxopropan-2-yl)carbamate A58.

TLC system: 100% Ethyl acetate; Rf: 0.3

LCMS (ESI): m/z 722.75 (M+H)⁺

### Example 2—Synthesis of Compounds of Formula (II)

### Synthesis of Compound B62

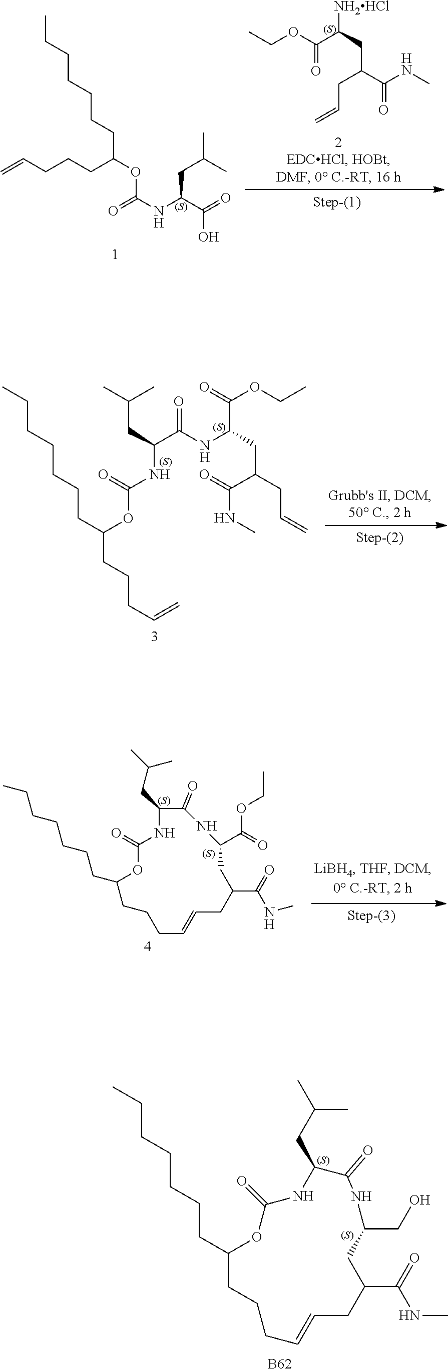

### Ethyl (2S)-2-((2S)-4-methyl-2-(((tridec-1-en-6-yloxy)carbonyl)amino)pentanamido)-4-(methylcarbamoyl)hept-6-enoate (3)

To a stirred solution of 2-benzyloct-7-enoic acid (1) (300 mg, 0.845 mmol) in DMF (3 mL) at 0° C. was added EDC.HCl (242 mg, 1.267 mmol), HOBt (172 mg, 1.267 mmol), DIPEA (0.4 mL, 2.53 mmol) and reaction mixture was stirred for 15 min. at same temperature then added ethyl (2S)-2-amino-4-(methylcarbamoyl)hept-6-enoate hydrochloride (2) (211 mg, 0.929 mmol) and stirred at RT for 16 h. After consumption of starting material, the reaction mixture was quenched with ice water (500 mL) and the resultant precipitate was filtered to afford pure compound Ethyl (2S)-2-((2S)-4-methyl-2-(((tridec-1-en-6-yloxy)carbonyl)amino)pentanamido)-4-(methylcarbamoyl)hept-6-enoate (3).

### Ethyl (4S,7S,E)-16-heptyl-4-isobutyl-9-(methylcarbamoyl)-2,5-dioxo-1-oxa-3,6-diazacyclohexadec-11-ene-7-carboxylate (4)

To a stirred solution of Ethyl (2S)-2-((2S)-4-methyl-2-(((tridec-1-en-6-yloxy)carbonyl)amino)pentanamido)-4-(methylcarbamoyl)hept-6-enoate (3) (300 mg, 0.530 mmol) in DCM (45 mL) was added Grubb's II (45 mg, 0.053 mmol) at rt and stirred at 50° C. for 2 h. After consumption of starting material reaction mixture was evaporated under reduced pressure to afford crude product; this crude was purified by normal phase chromatography with eluting 4% MeOH in DCM to afforded pure Ethyl (4S,7S,E)-16-heptyl-4-isobutyl-9-(methylcarbamoyl)-2,5-dioxo-1-oxa-3,6-diazacyclohexadec-11-ene-7-carboxylate (4).

### (4S,7S,E)-16-heptyl-7-(hydroxymethyl)-4-isobutyl-N-methyl-2,5-dioxo-1-oxa-3,6-diazacyclohexadec-11-ene-9-carboxamide B62

To a stirred solution of ethyl (4S,7S,E)-16-heptyl-4-isobutyl-9-(methylcarbamoyl)-2,5-dioxo-1-oxa-3,6-diazacyclohexadec-11-ene-7-carboxylate (4) (200 mg, 0.372 mmol) in DCM (2 mL) was added 2M LiBH₄ in THE (0.55 mL, 1.111 mmol) at 0° C., reaction mixture stirred for 2 h at rt. Reaction mixture was quenched with water (300 mL) and extracted with DCM (2×15 mL). Organic layer was washed with brine solution, dried over Na₂SO₄ and concentrated to get crude. Crude compound was purified by prep-HPLC to afford pure (4S,7S,E)-16-heptyl-7-(hydroxymethyl)-4-isobutyl-N-methyl-2,5-dioxo-1-oxa-3,6-diazacyclohexadec-11-ene-9-carboxamide B62.

TLC system: 10% MeOH in DCM; Rf: 0.3

LCMS (ESI): m/z 496.30 (M+H)⁺

### Synthesis of Compounds B63 and B64

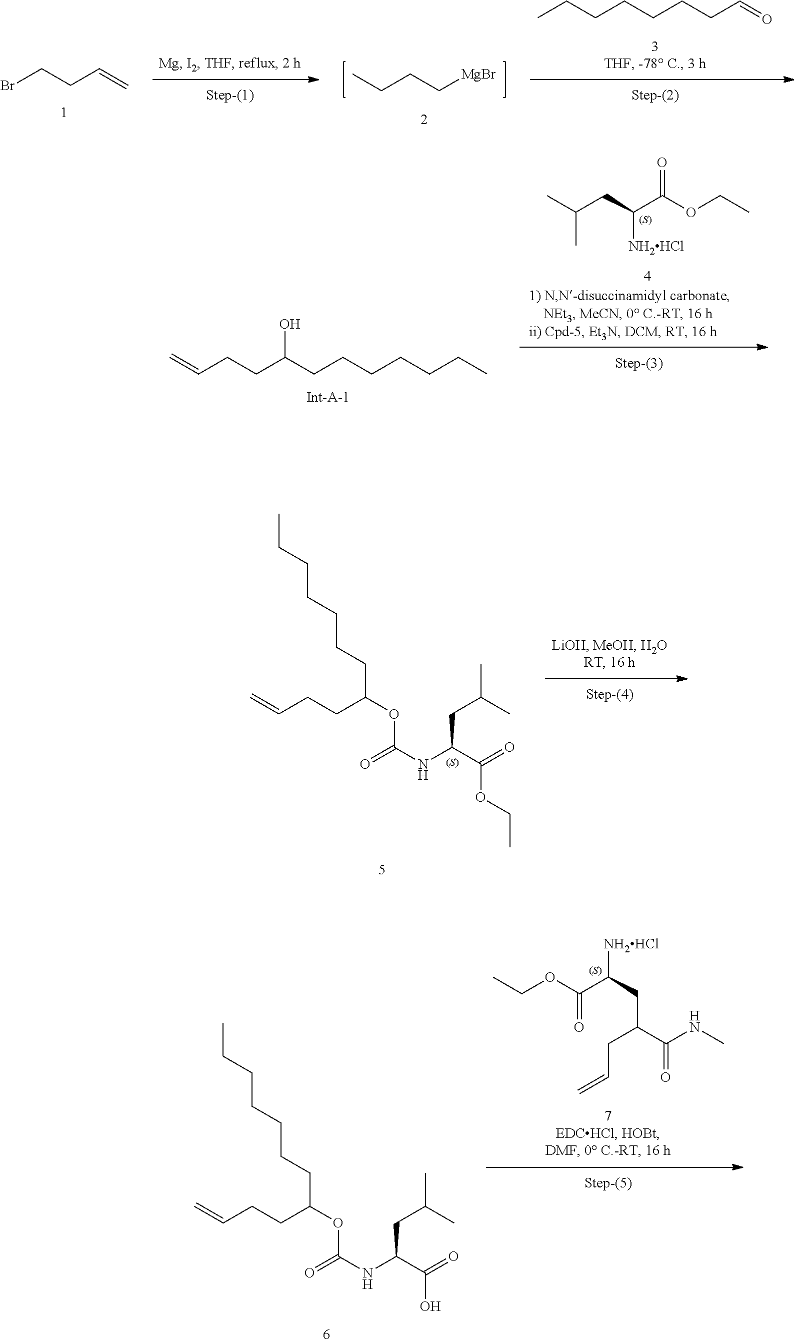

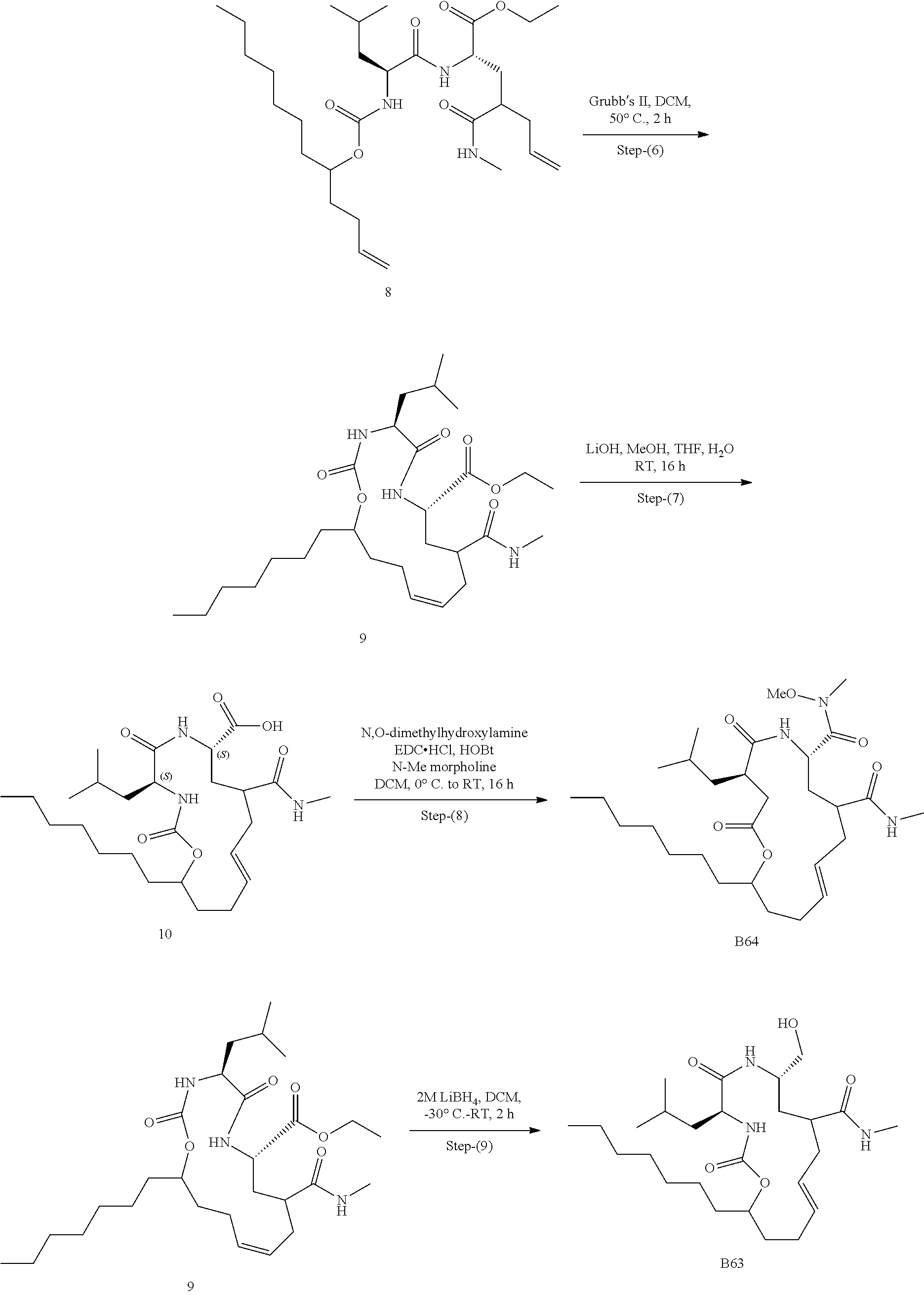

### Dodec-1-en-5-ol (Int-A-1)

To a dried magnesium turnings (1.7 g, 74.074 mmol) and catalytic iodine (189 mg, 074 mmol) in a three-neck RBF added dry diethyl ether (100 ml), was slowly added 4-bromobut-1-ene (10 g, 74.074 mmol) then 1,2-dibromo ethane (139 mg, 074 mmol) at RT. (Observed generation of exotherm and solvent reflux). Reaction mixture was stirred at RT for 2 h. The resultant heptylmagnesium bromide 2 15.07 g, 74.626 mmol) has been used directly in the next step.

To a stirred solution of octanal 3 (8 g, 62.5 mmol) in dry diethyl ether (40 mL) at −78° C. was slowly added above Grignard mixture heptylmagnesium bromide (2) (15.07 g, 74.626 mmol) drop wise. The reaction mixture was stirred at −78° C. for 2 h. The reaction mixture was quenched with sat. NH₄Cl (100 mL), extracted with EtOAc (3×250 mL), dried over anhydrous Na₂SO₄ and evaporated under vacuum. Obtained crude was purified by silica column eluting with 4% EtOAc and pet ether to afford dodec-1-en-5-ol Int-A-1.

### Ethyl ((dodec-1-en-5-yloxy)carbonyl)-L-leucinate (5)

To a stirred solution of dodec-1-en-5-ol Int-A-1 (1 g, 5.434 mmol) in acetonitrile (10 mL) added triethylamine (2.26 mL) at 0° C. and N,N-disuccinamidyl carbonate (1.87 g, 7.06 mmol) at 0° C. and stirred at room temperature for 16 h. Reaction mixture was evaporated under reduced pressure. Crude was diluted with ice water (25 mL), extracted with EtOAc (2×25 mL), dried over anhydrous Na₂SO₄ and evaporated under reduced pressure to obtain crude residue. It was taken in DCM (10 mL) added triethylamine (0.8 mL, 6.134 mmol)) and ethyl-L-leucinate hydrochloride (400 mg, 2.04 mmol) at 0° C. and stirred at room temperature for 16 h. The reaction mixture was quenched with ice water (50 mL) and extracted with DCM (50 mL×2). Organic layer was washed with brine solution (30 mL), dried over sodium sulfate and concentrated under reduced pressure. Crude was purified through silica gel (100-200) column chromatography by eluting with 1% EtOAc in pet ether to afford ethyl ((dodec-1-en-5-yloxy)carbonyl)-L-leucinate 5.

### ((dodec-1-en-5-yloxy)carbonyl)-L-leucine (6)

To a stirred solution of ethyl ((dodec-1-en-5-yloxy)carbonyl)-L-leucinate 5 (670 mg, 1.815 mmol) in dry THE (4 mL), methanol (2 mL) and water (1 mL), was added lithium hydroxide (132 mg, 5.49 mmol) at 0° C. and stirred at room temperature for 16 h. Reaction mixture distilled off under reduced pressure, crude compound acidified with 2N aq. HCl and adjusted pH ˜4, and extracted with EtOAc (2×200 mL), dried over anhydrous Na₂SO₄, and concentrated under reduced pressure to afford compound ((dodec-1-en-5-yloxy)carbonyl)-L-leucine (6).

### Ethyl (2S)-2-((2S)-2-(((dodec-1-en-5-yloxy)carbonyl)amino)-4-methylpentanamido)-4-(methylcarbamoyl)hept-6-enoate (8)

To a stirred solution of ((dodec-1-en-5-yloxy)carbonyl)-L-leucine (6) (2.5 g, 7.33 mmol) in DMF (35 mL), were added EDC.HCl (2.1 g, 10.99 mmol), HOBt (1.4 g, 10.99 mmol) and DIPEA (3.7 mL, 21.99 mmol) at 0° C. and stirred 10 min, was added ethyl (2S)-2-amino-4-(methylcarbamoyl)hept-6-enoate hydrochloride (7) (2.3 g, 8.78 mmol) at 0° C. and stirred at room temperature for 16 h. Reaction mixture was diluted with ice water (150 mL), extracted with ethyl acetate (2×150 mL), dried over anhydrous Na₂SO₄ and evaporated under reduced pressure. The crude residue was purified by column Grace normal phase with eluent 60% EtOAc in pet ether to afford ethyl (2S)-2-((2S)-2-(((dodec-1-en-5-yloxy)carbonyl)amino)-4-methylpentanamido)-4-(methylcarbamoyl)hept-6-enoate (8).

### Ethyl (4S,7S,Z)-15-heptyl-4-isobutyl-9-(methylcarbamoyl)-2,5-dioxo-1-oxa-3,6-diazacyclopentadec-11-ene-7-carboxylate (9)

To a stirred solution of ethyl (2S)-2-((2S)-2-(((dodec-1-en-5-yloxy)carbonyl)amino)-4-methylpentanamido)-4-(methylcarbamoyl)hept-6-enoate (8) (1.5 g, 2.722 mmol) in DCM (10 mL) was added Grubb's II catalyst (231 mg, 0.27 mmol) at room temperature and stirred at 50° C. for 2 h. Reaction mixture was quenched with water (25 mL) and extracted with DCM (2×50 mL). Combined organic layer were washed with brine solution (30 mL), dried over anhydrous Na₂SO₄ and concentrated under reduced pressure to afford crude residue. It was purified by column Grace normal phase using 60% of EtOAc in pet ether to afford ethyl (4S,7S,Z)-15-heptyl-4-isobutyl-9-(methylcarbamoyl)-2,5-dioxo-1-oxa-3,6-diazacyclopentadec-11-ene-7-carboxylate (9).

### (4S,7S,E)-15-heptyl-4-isobutyl-9-(methylcarbamoyl)-2,5-dioxo-1-oxa-3,6-diazacyclopentadec-11-ene-7-carboxylic acid (10)

To a stirred solution of ethyl (4S,7S,Z)-15-heptyl-4-isobutyl-9-(methylcarbamoyl)-2,5-dioxo-1-oxa-3,6-diazacyclopentadec-11-ene-7-carboxylate (9) (250 mg, 0.478 mmol) in THE (4 mL), methanol (2 mL) and water (1 mL), was added lithium hydroxide (45.8 mg, 1.912 mmol) at room temperature and stirred at same for 16 h. Reaction mixture completely distilled under reduced pressure, crude compound acidified with 2N HCl solution up to pH ˜4, and extracted with ethyl acetate (2×25 mL), dried over anhydrous Na₂SO₄, concentrated under reduced pressure to afford compound (4S,7S,E)-15-heptyl-4-isobutyl-9-(methylcarbamoyl)-2,5-dioxo-1-oxa-3,6-diazacyclopentadec-11-ene-7-carboxylic acid (10).

### (4S,7S,E)-15-heptyl-4-isobutyl-N⁷-methoxy-N⁷,N⁹-dimethyl-2,5-dioxo-1-oxa-3,6-diazacyclopentadec-11-ene-7,9-dicarboxamide B64

To a stirred solution of (4S,7S,E)-15-heptyl-4-isobutyl-9-(methylcarbamoyl)-2,5-dioxo-1-oxa-3,6-diazacyclopentadec-11-ene-7-carboxylic acid (10) (175 mg, 0.3535 mmol) in tetrahydrofuran (3 mL) and DCM (3 mL) added EDC.HCl (81 mg, 0.424 mmol), HOBT (57 mg, 0.4242 mmol), N-methylmorpholine (43 mg, 0.4242 mmol) and N,O-dimethylhydroxylamine (41 mg, 0.424 mmol) at 0° C. simultaneously and stirred at room temperature for 16 h. Reaction mixture was diluted with ice water (50 mL), extracted with EtOAc (2×50 mL), dried over anhydrous Na₂SO₄ and evaporated under reduced pressure. The crude residue was purified by prep HPLC to (4S,7S,E)-15-heptyl-4-isobutyl-N⁷-methoxy-N⁷,N⁹-dimethyl-2,5-dioxo-1-oxa-3,6-diazacyclopentadec-11-ene-7,9-dicarboxamide B64.

TLC system: 70% EtOAc in pet ether; Rf: 0.4

LCMS (ESI): m/z 539.32 (M+H)⁺

### (4S,7S,E)-15-heptyl-7-(hydroxymethyl)-4-isobutyl-N-methyl-2,5-dioxo-1-oxa-3,6-diazacyclopentadec-11-ene-9-carboxamide B63

To a stirred solution of ethyl (4S,7S,Z)-15-heptyl-4-isobutyl-9-(methylcarbamoyl)-2,5-dioxo-1-oxa-3,6-diazacyclopentadec-11-ene-7-carboxylate 9 (250 mg, 0.478 mmol) in dry DCM (5 mL) at −30° C. was added lithium borohydride (2 M in THF) (0.47 mL, 0.956 mmol) and stirred at same temperature for 2 h. Reaction mixture quenched with 2N aq. HCl and extracted with EtOAc (2×50 mL), dried over anhydrous Na₂SO₄ and evaporated under reduced pressure. The crude residue was purified by prep HPLC to afford pure (4S,7S,E)-15-heptyl-7-(hydroxymethyl)-4-isobutyl-N-methyl-2,5-dioxo-1-oxa-3,6-diazacyclopentadec-11-ene-9-carboxamide B63.

TLC system: 10% Methanol in dichloromethane; Rf: 0.5

LCMS (ESI): m/z 482.34 (M+H)⁺

### Synthesis of Compound B65

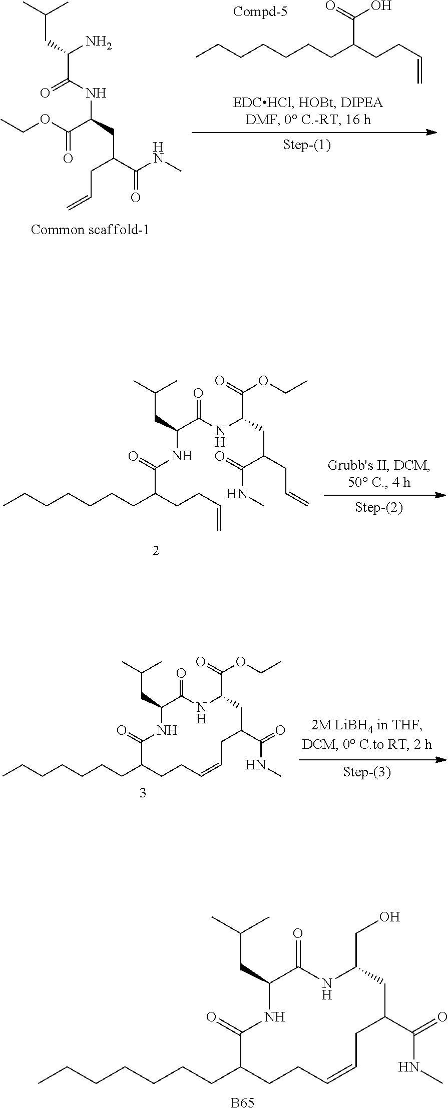

### Ethyl (2S)-2-((2S)-2-(2-(but-3-en-1-yl)nonanamido)-4-methylpentanamido)-4-(methylcarbamoyl)hept-6-enoate (2)

To a stirred solution of 2-(but-3-en-1-yl)nonanoic acid (1) (2.0 g, 6.13 mmol) (synthesis was reported in B63 & B64) in DMF (13 mL) at 0° C. was added EDC.HCl (1.7 g, 9.19 mmol), HOBt (1.2 g, 9.19 mmol) and DIPEA (3.3 mL, 18.39 mmol and stirred for 15 min, then added Ethyl (2S)-2-((S)-2-amino-4-methylpentanamido)-4-(methylcarbamoyl)hept-6-enoate (Common scaffold-1) (1.3 g, 6.13 mmol) at 0° C. and stirred at room temperature for 16 h. The progress of the reaction was monitored by TLC & LCMS. After consumption of starting material, reaction mixture was diluted with ice water (50 mL), extracted with EtOAc (2×70 mL), combined organic layer was washed with brine solution and dried over anhydrous Na₂SO₄, filtered and evaporated under reduced pressure. The crude residue was purified by silica gel (100-200) compound eluted using 1% methanol in DCM, to afford Ethyl (2S)-2-((2S)-2-(2-(but-3-en-1-yl)nonanamido)-4-methylpentanamido)-4-(methylcarbamoyl)hept-6-enoate (2).

### Ethyl (2S,5S,Z)-13-heptyl-2-isobutyl-7-(methylcarbamoyl)-3,14-dioxo-1,4-diazacyclotetradec-9-ene-5-carboxylate (3)

To a stirred solution of Ethyl (2S)-2-((2S)-2-(2-(but-3-en-1-yl)nonanamido)-4-methylpentanamido)-4-(methylcarbamoyl)hept-6-enoate (2) (1 g, 1.86 mmol) in DCM (150 mL) was added Grubb's II catalyst (157 mg, 0.18 mmol) at RT then heated to 50° C. for 2 h. The progress of the reaction was monitored by TLC & LCMS. After consumption of starting material, reaction mixture was cooled to RT then solvent was evaporated to afford crude residue. It was purified by Grace normal phase using 50% of EtOAc in pet ether to afford Ethyl (2S,5S,Z)-13-heptyl-2-isobutyl-7-(methylcarbamoyl)-3,14-dioxo-1,4-diazacyclotetradec-9-ene-5-carboxylate.

### (2S,5S,Z)-13-heptyl-5-(hydroxymethyl)-2-isobutyl-N-methyl-3,14-dioxo-1,4-diazacyclotetradec-9-ene-7-carboxamide B65

To a stirred solution of Ethyl (2S,5S,Z)-13-heptyl-2-isobutyl-7-(methylcarbamoyl)-3,14-dioxo-1,4-diazacyclotetradec-9-ene-5-carboxylate (3) (250 mg, 0.49 mmol) in DCM (5 mL) at 0° C. was slowly added drop wise 2M Lithium borohydride in THE (0.3 mL, 0.73 mmol). Then reaction mixture was stirred for 2 h at RT, The progress of the reaction was monitored by TLC & LCMS. After consumption of starting material, reaction mass was quenched with ice water (10 mL) and extracted with DCM (2×50 mL). Combined organic layer washed with brine solution then dried over anhydrous Na₂SO₄, filtered and evaporated under reduced pressure. The crude residue was purified by Prep HPLC, to afford (2S,5S,Z)-13-heptyl-5-(hydroxymethyl)-2-isobutyl-N-methyl-3,14-dioxo-1,4-diazacyclotetradec-9-ene-7-carboxamide B65.

TLC system: 5% MeOH in DCM; Rf: 0.2

LCMS (ESI): m/z 466.32 (M+H)⁺

### Synthesis of Compounds B1 and B66

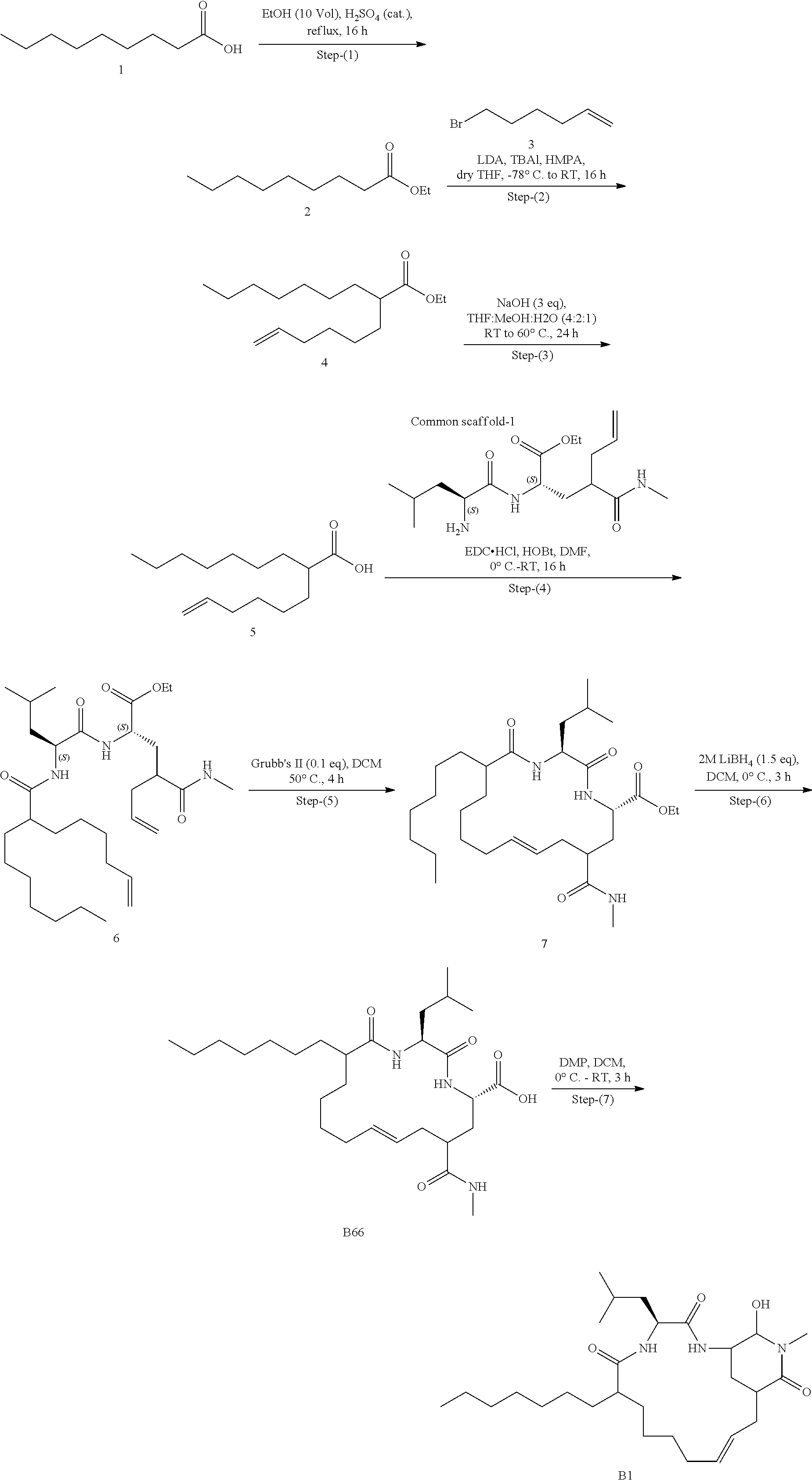

### Ethyl nonanoate (2)

To a stirred solution of nonanoic acid (1) (10 g, 63.29 mmol) in EtOH (100 mL) was added H₂SO₄ (2 mL) and heated to reflux for 16 h. After consumption of starting material, the reaction mixture was evaporated under reduced pressure to afford crude product; this crude was purified by column chromatography by using silica-gel (100-200) with eluting 2% EtOAc in hexane to afford ethyl nonanoate (2).

### Ethyl 2-(hex-5-en-1-yl)nonanoate (4)

At 0° C., to a stirred solution of freshly prepared LDA (n-Buli 15 mL, 37.63 mmol, DIPA 5.5 mL, 40.32 mmol in THF) was added ethyl nonanoate (2) (5 g, 26.88 mmol) at RT. Reaction mixture was cooled to −78° C. and stirred for 1 h then added TBAI (490 mg, 1.34 mmol), HMPA (5 mL) and 6-bromohex-1-ene (3) (7 g, 43.01 mmol) at same temperature. The reaction mixture was stirred at RT for 16 h. After consumption of starting material reaction mixture was quenched with sat. NH₄Cl solution (100 mL) and extracted with EtOAc (2×40 mL). Organic layer was washed with brine (50 mL) then dried over sodium sulfate and concentrated under reduced pressure to afford crude product; this crude was purified by column chromatography by using silica-gel (100-200) with eluting 2% EtOAc in hexane to afford pure compound ethyl 2-(hex-5-en-1-yl) nonanoate (4).

### 2-(Hex-5-en-1-yl)nonanoic acid (5)

To a stirred solution of ethyl 2-(hex-5-en-1-yl) nonanoate (4) (1 g, 3.73 mmol) in THF:methanol:water (3:2:1) (15 mL) was added NaOH (0.62 g, 11.19 mmol) and the mixture was heated to 60° C. for 24 h. After consumption of starting material, solvent was concentrated under reduced pressure to afford crude. The crude residue was acidified with 2N HCl and extracted with EtOAc (2×50 mL). Organic layer was washed with water (50 mL), brine (50 mL) dried over sodium sulfate then concentrated under reduced pressure to afford 2-(hex-5-en-1-yl)nonanoic acid (5). This product was used for next step without any purification.

### Ethyl (2S)-2-((2S)-2-(2-(hex-5-en-1-yl) nonanamido)-4-methylpentanamido)-4-(methylcarbamoyl) hept-6-enoate (7)

To a stirred solution of 2-(hex-5-en-1-yl)nonanoic acid (5) (0.5 g, 2.08 mmol) in DMF (10 mL) was added EDC.HCl (0.59 g, 3.12 mmol), HOBt (0.42 g, 3.12 mmol), DIPEA (1.1 mL, 6.24 mmol) at 0° C. and reaction mixture was stirred for 15 min at 0° C. then added ethyl (2S)-2-((S)-2-amino-4-methylpentanamido)-4-(methylcarbamoyl)hept-6-enoate (0.52 g, 2.29 mmol) at 0° C. and stirred for 16 h at rt. After consumption of starting material, the reaction mixture was quenched with ice water and extracted with EtOAc (2×30 mL). Organic layer was washed with brine (50 mL), dried over anhydrous Na₂SO₄ and concentrated under reduced pressure to afford crude product; this crude product was purified by column chromatography by using silica-gel (100-200) with eluting 40% EtOAc in hexane to afford pure compound ethyl (2S)-2-((2S)-2-(2-(hex-5-en-1-yl)nonanamido)-4-methylpentanamido)-4-(methylcarbamoyl)hept-6-enoate (6).

### Ethyl (2S,5S,E)-15-heptyl-2-isobutyl-7-(methylcarbamoyl)-3,16-dioxo-1,4-diazacyclohexadec-9-ene-5-carboxylate (7)

To a stirred solution of ethyl (2S)-2-((2S)-2-(2-(hex-5-en-1-yl) nonanamido)-4-methylpentanamido)-4-(methylcarbamoyl) hept-6-enoate (6) (0.2 g, 0.35 mmol) in DCM (30 mL) was added Grubb's II (0.03 g, 0.35 mmol) at rt and stirred at 50° C. for 4 h. After consumption of starting material reaction mixture was evaporated under reduced pressure to afford crude product; this crude was purified by normal phase chromatography with eluting 4% methanol in dichloromethane to afforded pure ethyl (2S,5S,E)-15-heptyl-2-isobutyl-7-(methylcarbamoyl)-3,16-dioxo-1,4-diazacyclohexadec-9-ene-5-carboxylate (7).

### (2S,5S,E)-15-heptyl-5-(hydroxymethyl)-2-isobutyl-N-methyl-3,16-dioxo-1,4-diazacyclohexadec-9-ene-7-carboxamide (8) B66

To a stirred solution of ethyl (2S, 5S, E)-15-heptyl-2-isobutyl-7-(methylcarbamoyl)-3, 16-dioxo-1, 4-diazacyclohexadec-9-ene-5-carboxylate (7) (500 mg, 0.934 mmol) in DCM (10 mL) was added 2M LiBH₄ in THE (1.4 mL, 2.08 mmol) at 0° C. and stirred for 3 h. After consumption of starting material reaction mixture was quenched with NH₄Cl (50 mL) and extracted with ethyl acetate (2×20 mL). Organic layers separated and dried over sodium sulfate then concentrated to afforded crude product. Crude product was purified by silica gel column chromatography by eluting with 5% MeOH in DCM to afford (2S,5S,E)-15-heptyl-5-(hydroxymethyl)-2-isobutyl-N-methyl-3,16-dioxo-1,4-diazacyclohexadec-9-ene-7-carboxamide (8) B66.

TLC system: 10% Methanol in DCM; Rf: 0.3

LCMS (ESI): m/z 494.32 (M+H)⁺

### (4S,E)-7-heptyl-18-hydroxy-4-isobutyl-17-methyl-2,5,17-triazabicyclo[13.3.1]nonadec-12-ene-3,6,16-trione B1

To a stirred solution of (2S,5S,E)-15-heptyl-5-(hydroxymethyl)-2-isobutyl-N-methyl-3,16-dioxo-1,4-diazacyclohexadec-9-ene-7-carboxamide (8) (100 mg, 0.202 mmol) in dichloromethane (5 mL) was added Dess-Martin periodinane (172 mg, 0.40 mmol) at 0° C. and reaction mixture was stirred at room temperature for 3 h. After consumption of starting material, added a solution of 10% aqueous sodium thiosulfate (20 mL), sat. NaHCO₃ solution (20 mL) and stirred for 15 min. The aqueous layer was separated, organic layer was washed with saturated aqueous NaHCO₃ (2×20 mL), water (2×20 mL), and brine (20 mL). Organic layer was dried over anhydrous Na₂SO₄ and concentrated to afford crude product; this crude was purified by prep HPLC to afforded (4S,E)-7-heptyl-18-hydroxy-4-isobutyl-17-methyl-2,5,17-triazabicyclo[13.3.1]nonadec-12-ene-3,6,16-trione B1.

TLC system: 10% Methanol in DCM; Rf: 0.1

LCMS (ESI): m/z 474.31 [M+H]⁺

### Synthesis of Compound B67

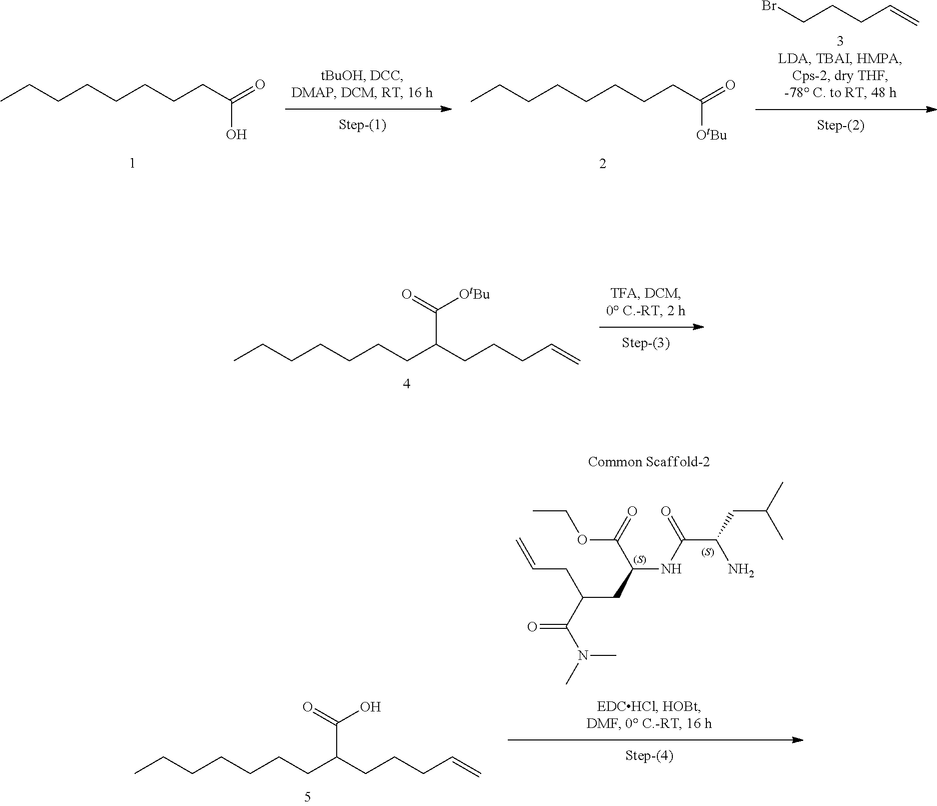

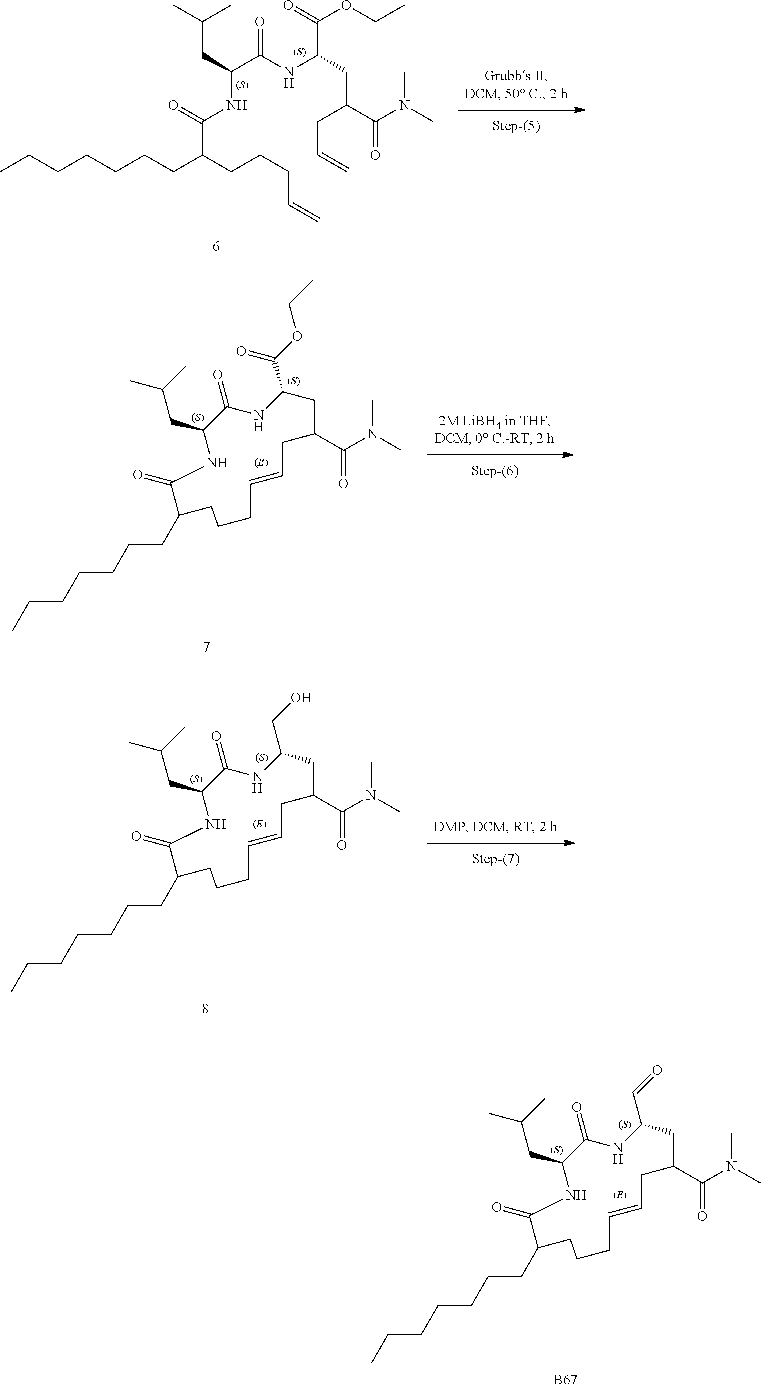

To a stirred solution of nonanoic acid (1) (50 g, 0.316 mmol) in DCM (500 mL) was added ^{t}BuOH (46.8 g, 632.9 mmol), DCC (65 g, 316.45 mmol), DMAP (7.7 g, 63.29 mmol) at RT and stirred for 16 h. After consumption of starting material, the reaction mixture was quenched with water (500 mL) and extracted with EtOAc (2×200 mL). Organic layer was washed with water (50 mL), brine (50 mL) dried over anhydrous Na₂SO₄, filtered and evaporated under reduced pressure to afford crude product. It was purified by silica gel (100-200 mesh) column chromatography by eluting with 100% pet ether to afford tert-butyl nonanoate (2).

### tert-butyl 2-(pent-4-en-1-yl)nonanoate (4)

To a stirred solution of freshly prepared LDA (n-BuLi 26 mL, 65.42 mmol), DIPA (10 mL, 70.09 mmol in THE (40 mL) at 0° C. was added tert-butyl nonanoate (2) (10 g, 46.72 mmol) at RT. Reaction mixture was cooled to −78° C. and stirred for 1 h then added TBAI (860 mg, 2.358 mmol), HMPA (10 mL) and 5-bromopent-1-ene (3) (8 mL, 74.76 mmol) at same temperature. The reaction mixture was stirred at RT for 16 h. After consumption of starting material reaction mixture was quenched with sat. NH₄Cl solution (150 mL) and extracted with EtOAc (2×50 mL). Combined organic layer was washed with brine (50 mL) then dried over anhydrous Na₂SO₄ and concentrated under reduced pressure to afford crude product; this crude was purified by silica gel (100-200 mesh) chromatography eluting with 2% EtOAc in hexane to afford tert-butyl 2-(pent-4-en-1-yl)nonanoate (4).

### 2-(Pent-4-en-1-yl) nonanoic acid (5)

To a stirred solution of tert-butyl 2-(pent-4-en-1-yl)nonanoate (4) (8 g, 28.36 mmol) in DCM (80 mL) was added TFA (80 mL, 10 vol) at 0° C. and mixture was stirred at RT for 4 h. After consumption of starting material, solvent was evaporated under reduced pressure to afford crude 2-(pent-4-en-1-yl) nonanoic acid (5) (5 g, 22.12 mmol). This crude was used for next step without any purification.

### Ethyl (2S)-4-(dimethylcarbamoyl)-2-((2S)-4-methyl-2-(2-(pent-4-en-1-yl) nonanamido) pentanamido) hept-6-enoate (6)

To a stirred solution of 2-(pent-4-en-1-yl) nonanoic acid (5) (900 mg, 4.22 mmol) in DMF (10 mL) at 0° C. was added EDC.HCl (968 mg, 5.07 mmol), HOBt (684 mg, 5.07 mmol), DIPEA (2.3 mL, 12.67 mmol) and reaction mixture was stirred for 15 min at same temperature then added ethyl (2S)-2-((S)-2-amino-4-methylpentanamido)-4-(dimethylcarbamoyl)hept-6-enoate (1.5 g, 4.22 mmol) and stirred at RT for 16 h. After consumption of starting material, the reaction mixture was quenched with ice water (50 mL) and extracted with EtOAc (2×30 mL). Organic layer was washed with brine (50 mL), dried over anhydrous Na₂SO₄ and concentrated under reduced pressure to afford crude product; this crude product was purified by silica gel (100-200 mesh) column chromatography eluting with 40% EtAOc in hexane to afford pure compound ethyl (2S)-4-(dimethylcarbamoyl)-2-((2S)-4-methyl-2-(2-(pent-4-en-1-yl) nonanamido) pentanamido) hept-6-enoate (6).

### Ethyl (2S,5S,E)-7-(dimethylcarbamoyl)-14-heptyl-2-isobutyl-3,15-dioxo-1,4-diazacyclopentadec-9-ene-5-carboxylate (7)

To a stirred solution of ethyl (2S)-4-(dimethylcarbamoyl)-2-((2S)-4-methyl-2-(2-(pent-4-en-1-yl) nonanamido) pentanamido) hept-6-enoate (6) (1.2 g, 2.12 mmol) in DCM (50 mL) was added Grubb's II (0.18 g, 0.21 mmol) at rt and stirred at 50° C. for 2 h. After consumption of starting material, reaction mixture was evaporated under reduced pressure to afford crude product; this crude was purified by normal phase chromatography with eluting 5% methanol in dichloromethane to afforded pure ethyl (2S,5S,E)-7-(dimethylcarbamoyl)-14-heptyl-2-isobutyl-3,15-dioxo-1,4-diazacyclopentadec-9-ene-5-carboxylate (7).

### (2S,5S,E)-14-heptyl-5-(hydroxymethyl)-2-isobutyl-N,N-dimethyl-3,15-dioxo-1,4-diazacyclopentadec-9-ene-7-carboxamide (8)

To a stirred solution of ethyl (2S,5S,E)-7-(dimethylcarbamoyl)-14-heptyl-2-isobutyl-3,15-dioxo-1,4-diazacyclopentadec-9-ene-5-carboxylate (7) (450 mg, 0.79 mmol) in DCM (10 mL) was added 2 M LiBH₄ in THE (1.19 mL, 2.38 mmol) at 0° C. and stirred at room temperature for 4 h. Reaction mixture quenched with 2N HCl and extracted with EtOAc (2×50 mL), dried over anhydrous Na₂SO₄ and evaporated under reduced pressure. The crude residue was purified by Grace normal phase using 5% MeOH/DCM to afford (2S,5S,E)-14-heptyl-5-(hydroxymethyl)-2-isobutyl-N,N-dimethyl-3,15-dioxo-1,4-diazacyclopentadec-9-ene-7-carboxamide (8).

### ((2S, 5S, E)-5-formyl-14-heptyl-2-isobutyl-N,N-dimethyl-3, 15-dioxo-1, 4-diazacyclopentadec-9-ene-7-carboxamide B67

To a stirred solution of (2S,5S,E)-14-heptyl-5-(hydroxymethyl)-2-isobutyl-N,N-dimethyl-3,15-dioxo-1,4-diazacyclopentadec-9-ene-7-carboxamide (8) (50 mg, 0.10 mmol) in DCM (2 mL) at 0° C. was added Dess-Martin periodinane (85 mg, 0.2 mmol and stirred at room temperature for 2 h. Reaction mixture was quenched with ice water (20 mL), extracted with DCM (2×20 mL), organic layer washed with sat. Hypo solution (3×20 mL), sat.NaHCO₃ (2×30 mL) and dried over anhydrous Na₂SO₄ and evaporated under reduced pressure. The crude residue was triturated with n-pentane to afford (2S,5S,E)-5-formyl-14-heptyl-2-isobutyl-N,N-dimethyl-3,15-dioxo-1,4-diazacyclopentadec-9-ene-7-carboxamide B67.

TLC system: 10% Methanol in DCM; Rf: 0.3

### Synthesis of Compounds B10 and B11

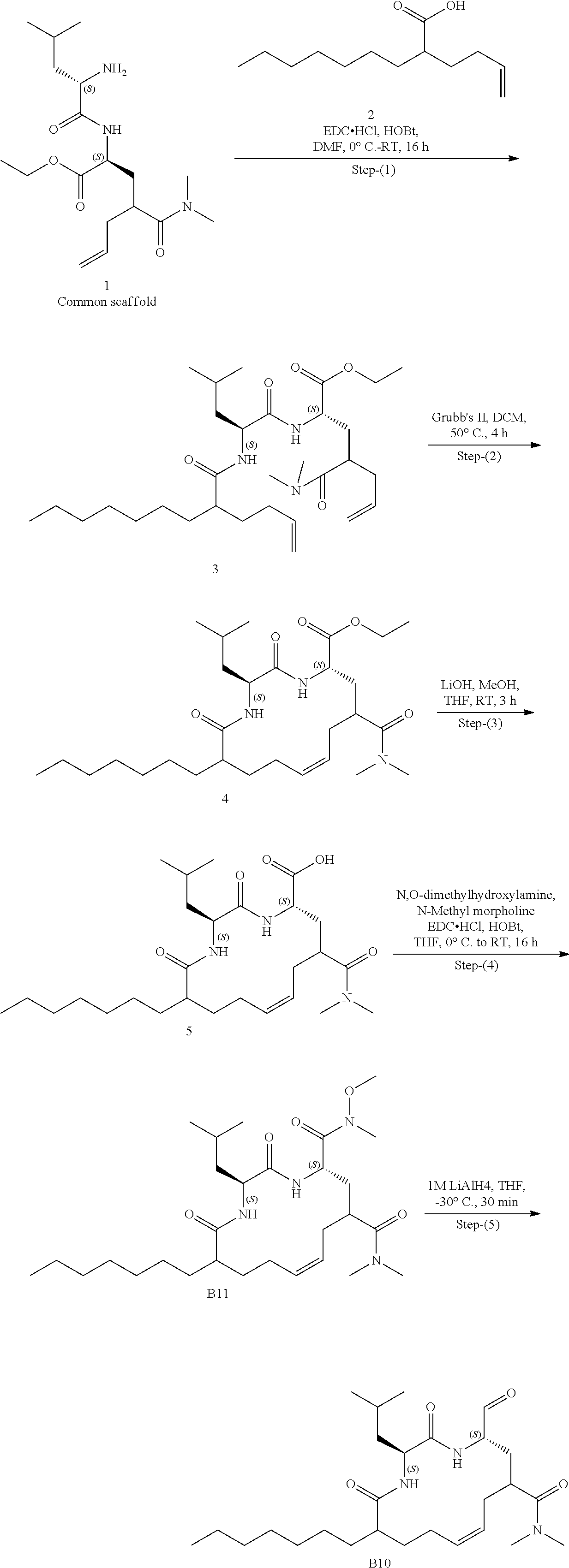

### Ethyl (2S)-2-((2S)-2-(2-(but-3-en-1-yl)nonanamido)-4-methylpentanamido)-4-(dimethylcarbamoyl)hept-6-enoate (3)

To a stirred solution of 2-(but-3-en-1-yl)nonanoic acid (2) (2 g, 9.43 mmol) in DMF (30 mL) was added EDC.HCl (2.72 g, 14.15 mmol), HOBt (1.91 g, 14.15 mmol), DIPEA (5.2 mL, 28.30 mmol) at 0° C. and reaction mixture was stirred for 15 min at same temperature then added Ethyl (2S)-2-((S)-2-amino-4-methylpentanamido)-4-(dimethylcarbamoyl)hept-6-enoate (3.68 g, 10.37 mmol) at 0° C. and stirred at RT for 16 h. After consumption of starting material, the reaction mixture was quenched with ice water (100 mL) and extracted with EtOAc (2×50 mL). Combined organic layer was washed with brine (100 mL), dried over anhydrous Na₂SO₄ and concentrated under reduced pressure to afford crude product; this crude product was purified by silica gel (100-200 mesh) column chromatography eluting with 60% EtAOc in hexane to afford Ethyl (2S)-2-((2S)-2-(2-(but-3-en-1-yl)nonanamido)-4-methylpentanamido)-4-(dimethylcarbamoyl)hept-6-enoate (3).

### Ethyl (2S,5S,Z)-7-(dimethylcarbamoyl)-13-heptyl-2-isobutyl-3,14-dioxo-1,4-diazacyclotetradec-9-ene-5-carboxylate (4)

To a stirred solution of Ethyl (2S)-2-((2S)-2-(2-(but-3-en-1-yl)nonanamido)-4-methylpentanamido)-4-(dimethylcarbamoyl)hept-6-enoate (3) (1 g, 1.82 mmol) in DCM (100 mL) was added Grubb's II (0.154 g, 0.18 mmol) at rt and stirred at 50° C. for 4 h. After consumption of starting material, reaction mixture was evaporated under reduced pressure to afford crude product; this crude was purified by normal phase chromatography with eluting 4% methanol in dichloromethane to afforded ethyl (2S,5S,Z)-7-(dimethylcarbamoyl)-13-heptyl-2-isobutyl-3,14-dioxo-1,4-diazacyclotetradec-9-ene-5-carboxylate (4).

### (2S,5S,Z)-7-(dimethylcarbamoyl)-13-heptyl-2-isobutyl-3,14-dioxo-1,4-diazacyclotetradec-9-ene-5-carboxylic acid (5)

To a stirred solution of ethyl (2S,5S,Z)-7-(dimethylcarbamoyl)-13-heptyl-2-isobutyl-3,14-dioxo-1,4-diazacyclotetradec-9-ene-5-carboxylate (4) (500 mg, 0.95 mmol) in THE (4 mL), MeOH (4 mL) and H₂O (1 mL), was added LiOH (0.046 g, 1.92 mmol) at rt and stirred at room temperature for 4 h. Reaction mixture was distilled off under reduced pressure, crude compound acidified to pH ˜4 with 2N HCl, and extracted with EtOAc (2×10 mL), dried over Na₂SO₄, concentrated under reduced pressure to afford (2S,5S,Z)-7-(dimethylcarbamoyl)-13-heptyl-2-isobutyl-3,14-dioxo-1,4-diazacyclotetradec-9-ene-5-carboxylic acid (5)

### (2S,5S,Z)-13-heptyl-2-isobutyl-N⁵-methoxy-N⁵,N⁷,N⁷-trimethyl-3,14-dioxo-1,4-diazacyclotetradec-9-ene-5,7-dicarboxamide (B11

To a stirred solution of (2S,5S,Z)-7-(dimethylcarbamoyl)-13-heptyl-2-isobutyl-3,14-dioxo-1,4-diazacyclotetradec-9-ene-5-carboxylic acid (5) (400 mg, 0.81 mmol) in THE (5 mL) and DCM (5 mL) at 0° C. was added EDC.HCl (186 mg, 0.97 mmol), HOBT (131 mg, 0.97 mmol), N-methylmorpholine (98 mg, 0.97 mmol) and N,O-dimethyl hydroxylamine (94 mg, 0.97 mmol) sequentially and stirred at room temperature for 16 h. Reaction mixture was diluted with ice water (20 mL), extracted with EtOAc (2×20 mL), dried over anhydrous Na₂SO₄ and evaporated under reduced pressure. The crude residue was purified by grace RP, by using 60% MeCN in 0.1% formic acid in water as eluent to afford (2S,5S,Z)-13-heptyl-2-isobutyl-N-methoxy-N⁵,N⁷,N⁷-trimethyl-3,14-dioxo-1,4-diazacyclotetradec-9-ene-5,7-dicarboxamide B11.

TLC system: 10% Methanol in DCM; Rf: 0.3

LCMS (ESI): m/z 537.62 [M+H]⁺

### (2S,5S,Z)-5-formyl-13-heptyl-2-isobutyl-N,N-dimethyl-3,14-dioxo-1,4-diazacyclotetradec-9-ene-7-carboxamide B10

To a stirred solution of (2S,5S,Z)-13-heptyl-2-isobutyl-N-methoxy-N⁵,N⁷,N⁷-trimethyl-3,14-dioxo-1,4-diazacyclotetradec-9-ene-5,7-dicarboxamide B11 (50 mg, 0.09 mmol) in THE (2 mL) was added 2M LiAlH₄ (0.05 mL, 0.09 mmol) at −30° C. and reaction mixture was stirred at −30° C. for 1 h. After consumption of starting material, diluted with ice water (5 mL) extracted with EtOAc (2×5 mL), organic layer was washed with water (2×5 mL), and brine (5 mL) and dried over anhydrous Na2SO4. Organic layer was filtered and evaporated under reduced pressure to afford (2S,5S,Z)-5-formyl-13-heptyl-2-isobutyl-N,N-dimethyl-3,14-dioxo-1,4-diazacyclotetradec-9-ene-7-carboxamide B11.

TLC system: 10% Methanol in DCM; Rf: 0.2

LCMS (ESI): m/z 478.31 [M+H]⁺

### Synthesis of Compounds B12 and B15

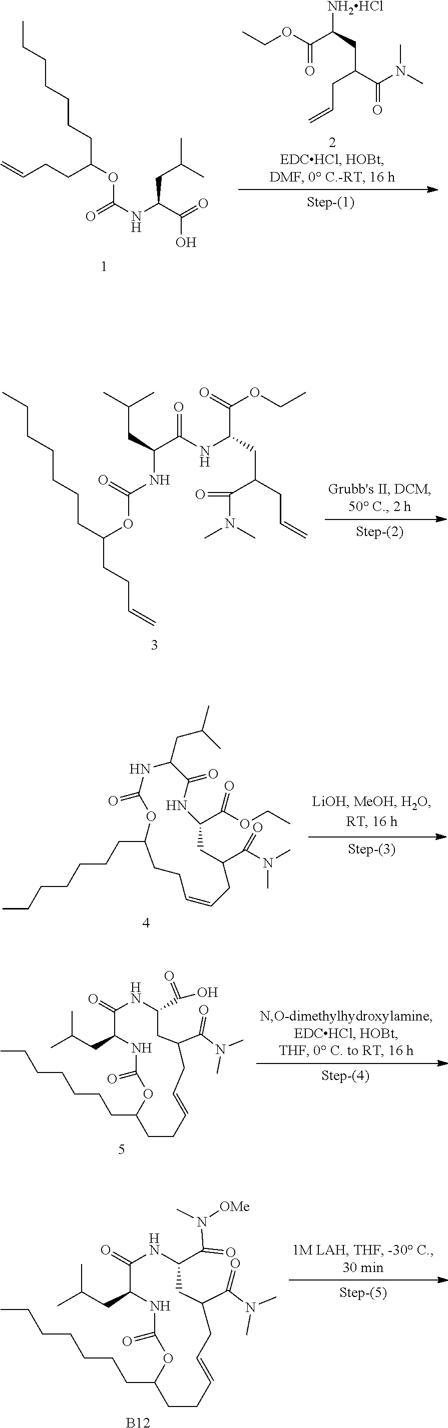

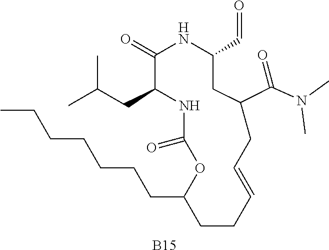

### Ethyl (2S)-2-((2S)-2-(((dodec-1-en-5-yloxy)carbonyl)amino)-4-methylpentanamido)-4-(methylcarbamoyl)hept-6-enoate (3)

To a stirred solution of ((dodec-1-en-5-yloxy)carbonyl)-L-leucine (1) (1.5 g, 4.39 mmol) in DMF (40 mL), were added EDC.HCl (1.26 g, 6.59 mmol), HOBt (0.89 g, 6.59 mmol) and Et₃N (3 mL, 21.99 mmol) at 0° C. and stirred for 10 min, then added ethyl (2S)-2-amino-4-(dimethylcarbamoyl)hept-6-enoate hydrochloride (2) (1.46 g, 5.27 mmol) at 0° C. and stirred at room temperature for 16 h. Reaction mixture was diluted with ice water (150 mL), extracted with EtOAc (2×150 mL) organic layer dried over anhydrous Na₂SO₄ and evaporated under reduced pressure. The crude residue was purified by Grace normal phase using 60% EtOAc in pet ether to afford ethyl (2S)-2-((2S)-2-(((dodec-1-en-5-yloxy)carbonyl)amino)-4-methylpentanamido)-4-(methylcarbamoyl)hept-6-enoate (3).

### Ethyl (4S,7S,Z)-9-(dimethylcarbamoyl)-15-heptyl-4-isobutyl-2,5-dioxo-1-oxa-3,6-diazacyclopentadec-11-ene-7-carboxylate (4)

To a stirred solution of ethyl (2S)-2-((2S)-2-(((dodec-1-en-5-yloxy)carbonyl)amino)-4-methylpentanamido)-4-(methylcarbamoyl)hept-6-enoate (3) (2 g, 3.539 mmol) in DCM (10 mL) was added Grubb's II catalyst (300 mg, 0.353 mmol) then heated to 50° C. for 2 h. Reaction mixture was quenched with water (25 mL) and extracted with DCM (2×50 mL). Combined organic layer were washed with brine solution (30 mL), dried over anhydrous Na₂SO₄ and concentrated under reduced pressure to afford crude residue. It was purified by Grace normal phase using 60% of EtOAc in pet ether to afford ethyl (4S, 7S, Z)-9-(dimethylcarbamoyl)-15-heptyl-4-isobutyl-2,5-dioxo-1-oxa-3,6-diazacyclopentadec-11-ene-7-carboxylate (4).

### (4S,7S,E)-9-(dimethylcarbamoyl)-15-heptyl-4-isobutyl-2,5-dioxo-1-oxa-3,6-diazacyclopentadec-11-ene-7-carboxylic acid (5)

To a stirred solution of ethyl (4S,7S,Z)-9-(dimethylcarbamoyl)-15-heptyl-4-isobutyl-2,5-dioxo-1-oxa-3,6-diazacyclopentadec-11-ene-7-carboxylate (4) (1.1 g, 2.048 mmol) in THE (8 mL), methanol (2 mL) and water (1 mL), was added lithium hydroxide (196 mg, 8.193 mmol) at room temperature and stirred at same for 16 h. Reaction mixture completely distilled under reduced pressure, crude compound acidified with 2N HCl solution up to pH ˜4, and extracted with EtOAc (2×25 mL), dried over anhydrous Na₂SO₄, concentrated under reduced pressure to afford 9-(dimethylcarbamoyl)-15-heptyl-4-isobutyl-2,5-dioxo-1-oxa-3,6-diazacyclopentadec-11-ene-7-carboxylic acid (5).

### (4S,7S,E)-15-heptyl-4-isobutyl-N⁷-methoxy-N⁷,N⁹,N⁹-trimethyl-2,5-dioxo-1-oxa-3,6-diazacyclopentadec-11-ene-7,9-dicarboxamide B12

To a stirred solution of 9-(dimethylcarbamoyl)-15-heptyl-4-isobutyl-2,5-dioxo-1-oxa-3,6-diazacyclopentadec-11-ene-7-carboxylic acid 5 (150 mg, 0.29 mmol) in THE (3 mL) and DCM (3 mL) added EDC.HCl (84 mg, 0.44 mmol), HOBT (59 mg, 0.44 mmol) N,O-dimethylhydroxylamine (34 mg, 0.35 mmol) at 0° C. simultaneously and stirred at room temperature for 16 h. Reaction mixture was diluted with ice water (20 mL), extracted with ethyl acetate (2×25 mL), dried over anhydrous Na₂SO₄ and evaporated under reduced pressure. The crude residue was purified by prep afforded (4S,7S,E)-15-heptyl-4-isobutyl-N⁷-methoxy-N⁷,N⁹,N⁹-trimethyl-2,5-dioxo-1-oxa-3,6-diazacyclopentadec-11-ene-7,9-dicarboxamide B12.

TLC system: 10% Methanol/DCM; Rf: 0.2

LCMS (ESI): m/z 553.47 (M+H)⁺

### (4S,7S,E)-7-formyl-15-heptyl-4-isobutyl-N,N-dimethyl-2,5-dioxo-1-oxa-3,6-diazacyclopentadec-11-ene-9-carboxamide B15

To a stirred solution of (4S,7S,E)-15-heptyl-4-isobutyl-N7-methoxy-N7,N9,N9-trimethyl-2,5-dioxo-1-oxa-3,6-diazacyclopentadec-11-ene-7,9-dicarboxamide B12 (60 mg, 0.108 mmol) in DCM (5 mL) at −30° C. added lithium borohydride (2 M in THF) (0.1 mL, 0.217 mmol) and stirred at same temperature for 2 h. Reaction mixture quenched with 2N HCl and extracted with EtOAc (2×20 mL), dried over Na₂SO₄ and evaporated under reduced pressure. The crude residue was purified by prep HPLC to afford (4S,7S,E)-7-formyl-15-heptyl-4-isobutyl-N,N-dimethyl-2,5-dioxo-1-oxa-3,6-diazacyclopentadec-11-ene-9-carboxamide B15.

TLC system: 10% MeOH/DCM; Rf: 0.5

LCMS (ESI): m/z 494.31 (M+H)⁺

### Synthesis of Compounds B13 and B16

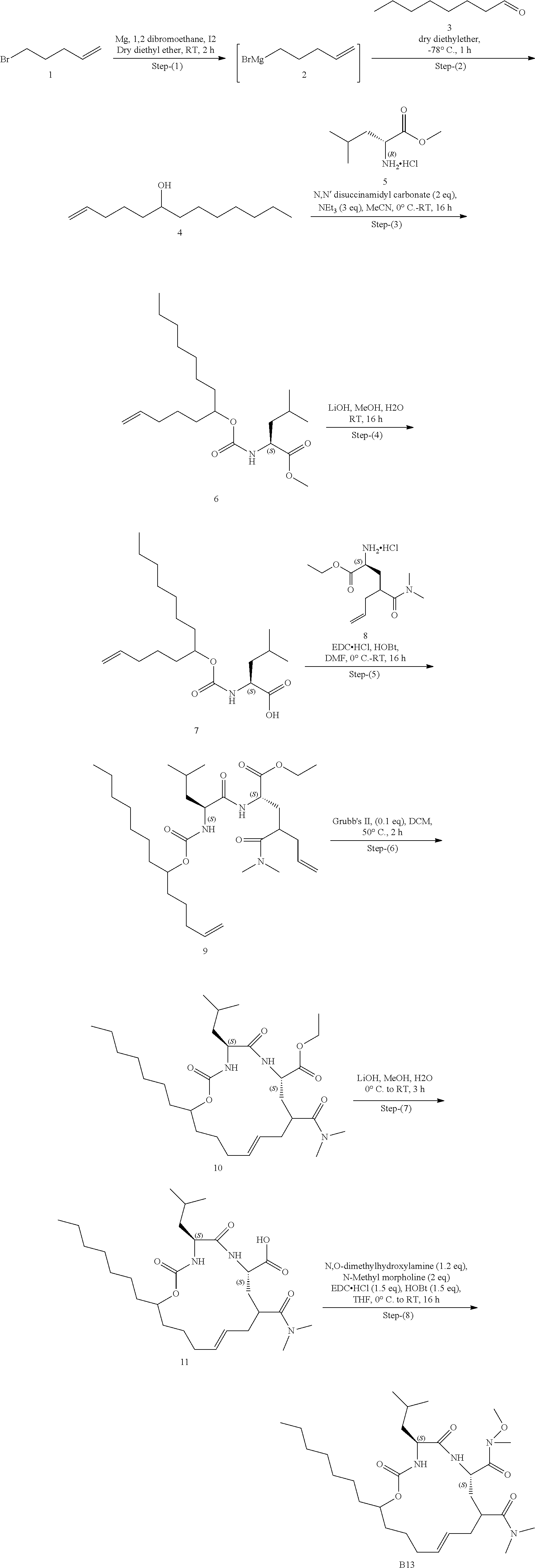

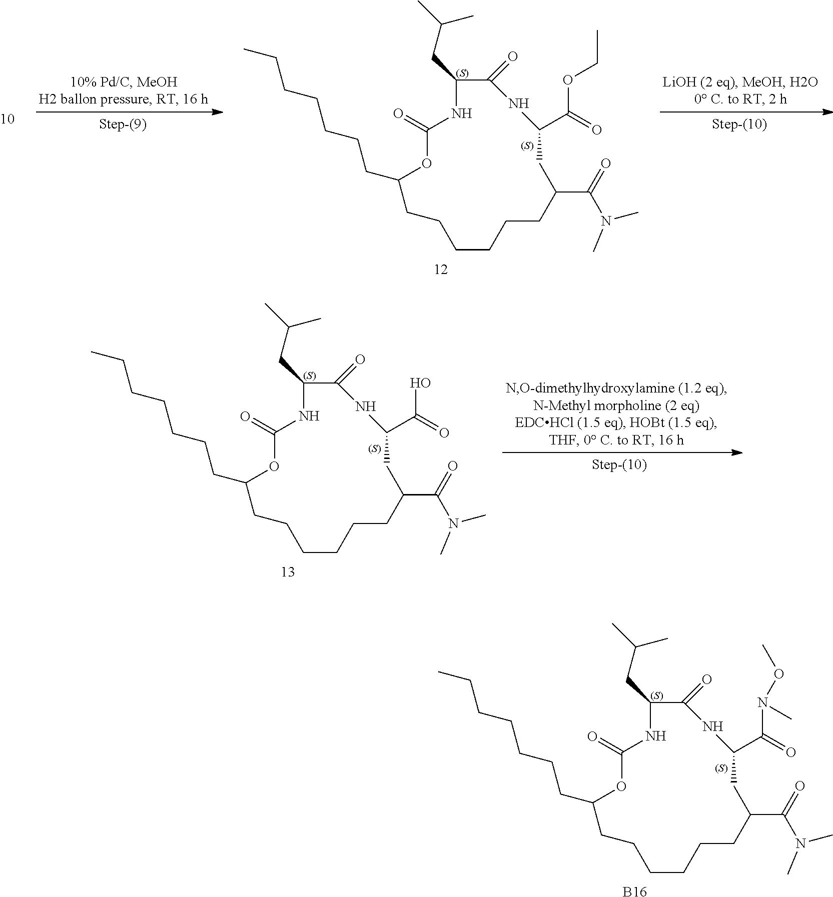

### Tridec-1-en-6-ol (4)

To a dried magnesium turnings (0.805 g, 33.55 mmol) and catalytic iodine (189 mg, 074 mmol) in a multi-neck RBF added dry diethyl ether (100 mL), 1,2-dibromo ethane (139 mg, 074 mmol) and 5-bromopent-1-ene 1 (4.3 g, 33.5 mmol) at RT. While maintaining reaction at room temperature self-vigorous reflux observed. Reaction mixture was stirred at RT for 2 h and the resultant Grignard mixture was used as such in the next step.

To a stirred solution of octanal 3 (5 g, 33.55 mmol) in dry diethyl ether (40 mL) at −78° C. was added slowly added above freshly prepared pent-4-en-1-ylmagnesium bromide (2) (5.81 g, 33.58 mmol) drop wise. The reaction mixture was stirred at same temperature for 1 h. The reaction mixture was quenched with sat. NH₄Cl (100 mL), extracted with EtOAc (2×200 mL), dried over anhydrous Na₂SO₄ and evaporated under vacuum. Obtained crude was purified by silica gel (100-200 mesh) column eluting with 2% EtOAc and petether to afford tridec-1-en-6-ol-4. Methyl ((tridec-1-en-6-yloxy)carbonyl)-L-leucinate (6)

To a stirred solution of tridec-1-en-6-ol-4 (4 g, 20.20 mmol) in acetonitrile (80 mL) added NEt₃ (13.8 mL) at rt and N,N-disuccinamidyl carbonate (10.7 g, 40.40 mmol) at rt and stirred at room temperature for 16 h. Reaction mixture was evaporated under reduced pressure. Crude was diluted with ice water (100 mL), extracted with EtOAc (2×50 mL), dried over anhydrous Na₂SO₄ and evaporated under reduced pressure obtained crude. The crude residue was taken in DCM (120 mL) added NEt₃ (12.4 mL, 88.49 mmol)) and methyl L-leucinate hydrochloride (6.4 g, 35.39 mmol) at 0° C. and stirred at room temperature for 16 h. The reaction mixture was quenched with ice water (100 mL) and extracted with DCM (100 mL×2). Organic layer was washed with brine solution (60 mL), dried over anhydrous Na₂SO₄ and concentrated under reduced pressure. Crude was purified through 100-200 silica gel column chromatography by eluting with 2% EtOAc in pet ether to afford methyl ((tridec-1-en-6-yloxy)carbonyl)-L-leucinate 6.

### ((Tridec-1-en-6-yloxy)carbonyl)-L-leucine (7)

To a stirred solution of methyl ((tridec-1-en-6-yloxy)carbonyl)-L-leucinate 6 (10 g, 27.10 mmol) in THE (60 mL), methanol (60 mL) and water (30 mL), was added lithium hydroxide (1.9 g, 81.30 mmol) at rt and stirred at room temperature for 2 h. Reaction mixture completely distilled under reduced pressure, crude compound acidified with 2N HCl solution up to pH ˜4, and extracted with EtOAc (2×300 mL), dried over anhydrous Na₂SO₄, concentrated under reduced pressure to afford compound ((tridec-1-en-6-yloxy)carbonyl)-L-leucine 7.

### Ethyl (2S)-4-(dimethylcarbamoyl)-2-((2S)-4-methyl-2-(((tridec-1-en-6-yloxy)carbonyl)amino)pentanamido)hept-6-enoate (9)

To a stirred solution of ((tridec-1-en-6-yloxy)carbonyl)-L-leucine 7 (3 g, 7.33 mmol) in DMF (35 mL), were added EDC.HCl (2.4 g, 12.67 mmol), HOBT (1.7 g, 12.67 mmol) and DIPEA (4.4 mL, 25.35 mmol) at 0° C. and stirred 10 min, was added ethyl (2S)-2-amino-4-(dimethylcarbamoyl)hept-6-enoate hydrochloride (8) (2.3 g, 8.45 mmol) at 0° C. and stirred at room temperature for 16 h. Reaction mixture was diluted with ice water (150 mL), extracted with EtOAc (2×150 mL), dried over anhydrous Na₂SO₄ and evaporated under reduced pressure. The crude residue was purified by column Grace normal phase with eluent 60% EtOAc in pet ether to afford ethyl (2S)-4-(dimethylcarbamoyl)-2-((2S)-4-methyl-2-(((tridec-1-en-6-yloxy)carbonyl)amino)pentanamido)hept-6-enoate 9.

### Ethyl (4S,7S,E)-9-(dimethylcarbamoyl)-16-heptyl-4-isobutyl-2,5-dioxo-1-oxa-3,6-diazacyclohexadec-11-ene-7-carboxylate (10)

To a stirred solution of ethyl (2S)-4-(dimethylcarbamoyl)-2-((2S)-4-methyl-2-(((tridec-1-en-6-yloxy)carbonyl)amino)pentanamido)hept-6-enoate 9 (1 g, 1.727 mmol) in DCM (100 mL) was added Grubb's II catalyst (146 mg, 0.172 mmol) at room temperature and stirred at 50° C. for 2 h. Reaction mixture was quenched with water (25 mL) and extracted with DCM (2×50 mL). Combined organic layers were washed with brine solution (30 mL), dried over anhydrous Na₂SO₄ and concentrated under reduced pressure to afford crude. Crude was purified by Grace normal Phase with eluent 3% methanol in DCM to afford ethyl (4S,7S,E)-9-(dimethylcarbamoyl)-16-heptyl-4-isobutyl-2,5-dioxo-1-oxa-3,6-diazacyclohexadec-11-ene-7-carboxylate 10.

### (4S,7S,E)-9-(Dimethylcarbamoyl)-16-heptyl-4-isobutyl-2,5-dioxo-1-oxa-3,6-diazacyclohexadec-11-ene-7-carboxylic acid (11)

To a stirred solution of ethyl (4S,7S,E)-9-(dimethylcarbamoyl)-16-heptyl-4-isobutyl-2,5-dioxo-1-oxa-3,6-diazacyclohexadec-11-ene-7-carboxylate (10) (700 mg, 1.26 mmol) in THE (5 mL), methanol (5 mL) and water (2.5 mL), was added lithium hydroxide (91 mg, 3.81 mmol) at room temperature and stirred at same for 3 h. Reaction mixture completely distilled under reduced pressure, crude compound acidified with 2N HCl solution up to pH ˜4, and extracted with EtOAc (2×25 mL), dried over anhydrous Na₂SO₄, concentrated under reduced pressure to afford compound (4S,7S,E)-9-(dimethylcarbamoyl)-16-heptyl-4-isobutyl-2,5-dioxo-1-oxa-3,6-diazacyclohexadec-11-ene-7-carboxylic acid (11).

### (4S,7S,E)-16-Heptyl-4-isobutyl-N⁷-methoxy-N⁷,N⁹,N⁹-trimethyl-2,5-dioxo-1-oxa-3,6-diazacyclohexadec-11-ene-7,9-dicarboxamide B13

To a stirred solution of (4S,7S,E)-9-(dimethylcarbamoyl)-16-heptyl-4-isobutyl-2,5-dioxo-1-oxa-3,6-diazacyclohexadec-11-ene-7-carboxylic acid 11 (700 mg, 1.338 mmol) in THE (7 mL) and DCM (7 mL) added EDC.HCl (306 m g, 1.606 mmol), HOBT (218 mg, 1.606 mmol), N-methylmorpholine (162 mg, 1.606 mmol) and N,O-dimethylhydroxylamine (156 mg, 1.606 mmol) at 0° C. simultaneously and stirred at room temperature for 16 h. Reaction mixture was diluted with ice water (50 mL), extracted with EtOAc (2×25 mL), dried over anhydrous Na₂SO₄ and evaporated under reduced pressure. The crude residue was purified by prep-HPLC to (4S,7S,E)-16-heptyl-4-isobutyl-N⁷-methoxy-N⁷,N⁹,N⁹-trimethyl-2,5-dioxo-1-oxa-3,6-diazacyclohexadec-11-ene-7,9-dicarboxamide B13.

TLC system: 100% EtOAc; Rf: 0.4

LCMS (ESI): m/z 567.36 (M+H)⁺

### Ethyl (4S,7S)-9-(dimethylcarbamoyl)-16-heptyl-4-isobutyl-2,5-dioxo-1-oxa-3,6-diazacyclohexadecane-7-carboxylate (12)

To a stirred solution of ethyl (4S,7S,E)-9-(dimethylcarbamoyl)-16-heptyl-4-isobutyl-2,5-dioxo-1-oxa-3,6-diazacyclohexadec-11-ene-7-carboxylate 10 (900 mg, 1.633 mmol) in MeOH (9 mL) added 10% Pd/C (100 mg) and stirred at H₂ atmosphere for 16 h. After consumption of starting material. Reaction mixture filtered through the celite bed washed with MeOH (100 mL) the filtrate was evaporated under reduced pressure to afford crude ethyl (4S,7S)-9-(dimethylcarbamoyl)-16-heptyl-4-isobutyl-2,5-dioxo-1-oxa-3,6-diazacyclohexadecane-7-carboxylate. (4S,7S)-9-(dimethylcarbamoyl)-16-heptyl-4-isobutyl-2,5-dioxo-1-oxa-3,6-diazacyclohexadecane-7-carboxylic acid (13)

To a stirred solution of ethyl (4S,7S)-9-(dimethylcarbamoyl)-16-heptyl-4-isobutyl-2,5-dioxo-1-oxa-3,6-diazacyclohexadecane-7-carboxylate 12 (800 mg, 1.44 mmol) in THE (5 mL), methanol (5 mL) and water (2.5 mL), was added lithium hydroxide (104 mg, 4.336 mmol) at room temperature and stirred at same for 2 h. Reaction mixture completely distilled under reduced pressure, crude compound acidified with 2N HCL solution up to pH ˜4, and extracted with ethyl acetate (2×25 mL), dried over anhydrous Na₂SO₄, concentrated under reduced pressure to afford compound (4S,7S)-9-(dimethylcarbamoyl)-16-heptyl-4-isobutyl-2,5-dioxo-1-oxa-3,6-diazacyclohexadecane-7-carboxylic acid 13.

### (4S,7S)-16-Heptyl-4-isobutyl-N⁷-methoxy-N⁷,N⁹,N⁹-trimethyl-2,5-dioxo-1-oxa-3,6-diazacyclohexadecane-7,9-dicarboxamide B16

To a stirred solution of (4S,7S)-9-(dimethylcarbamoyl)-16-heptyl-4-isobutyl-2,5-dioxo-1-oxa-3,6-diazacyclohexadecane-7-carboxylic acid (13) (750 mg, 1.42 mmol) in THE (3.75 mL) and DCM (3.75 mL) at 0° C. added EDC.HCl (327 mg, 1.714 mmol), HOBT (233 mg, 1.714 mmol), N-methylmorpholine (0.173 mg, 1.714 mmol) and N,O-dimethylhydroxylamine (167 mg, 1.714 mmol) and stirred at room temperature for 16 h. Reaction mixture was diluted with ice water (75 mL), extracted with EtOAc (2×50 mL), dried over anhydrous Na₂SO₄ and evaporated under reduced pressure. The crude residue was purified by prep HPLC to (4S,7S)-16-heptyl-4-isobutyl-N⁷-methoxy-N⁷,N⁹,N⁹-trimethyl-2,5-dioxo-1-oxa-3,6-diazacyclohexadecane-7,9-dicarboxamide B16.

TLC system: 70% EtOAc in pet ether; Rf: 0.4

LCMS (ESI): m/z 539.32 (M+H)⁺

### Synthesis of Compounds B20 and B14

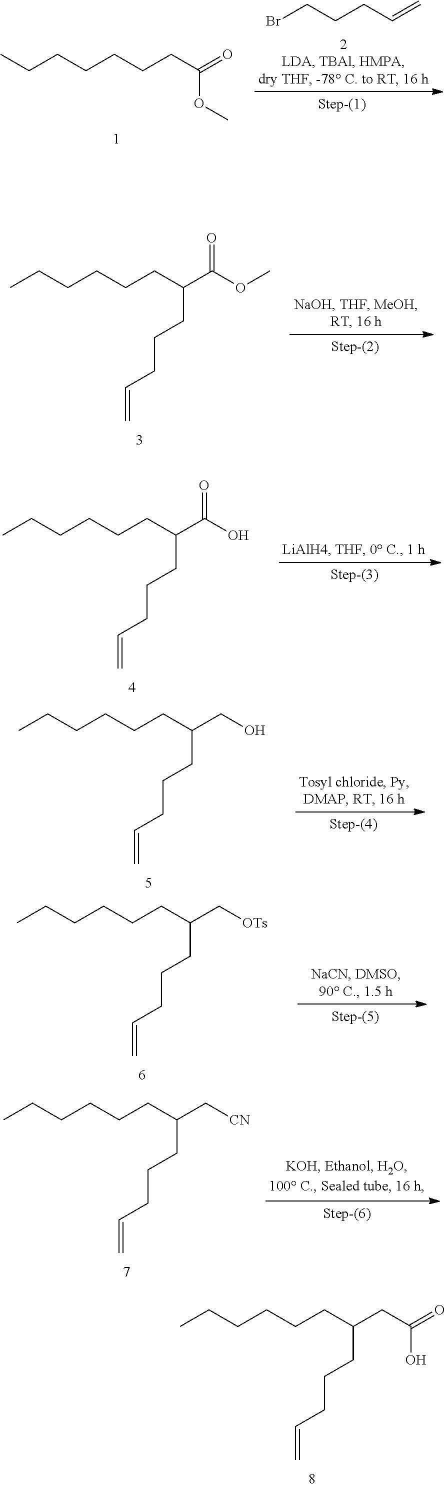

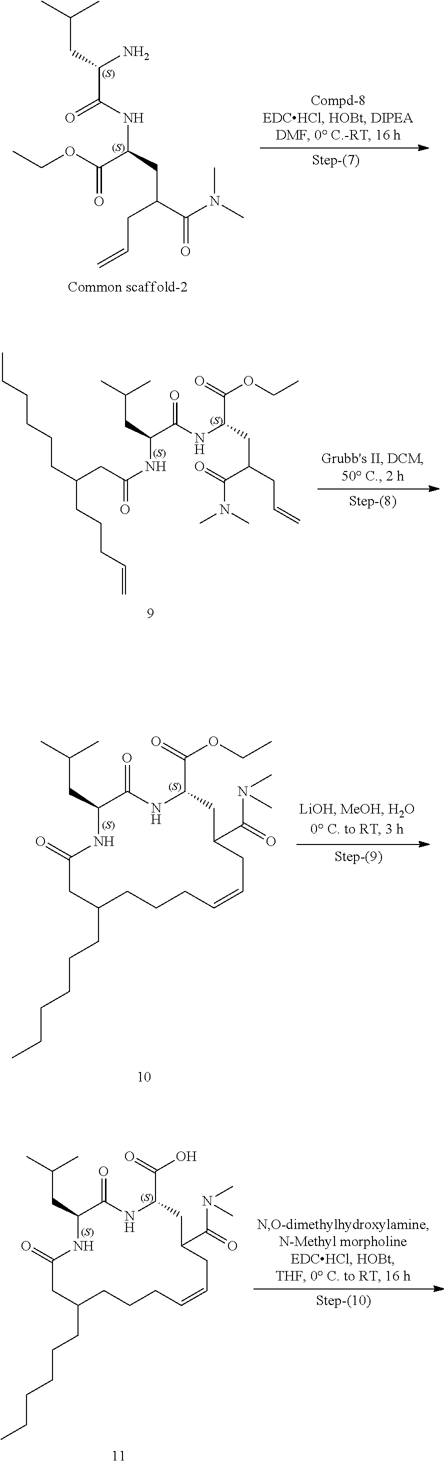

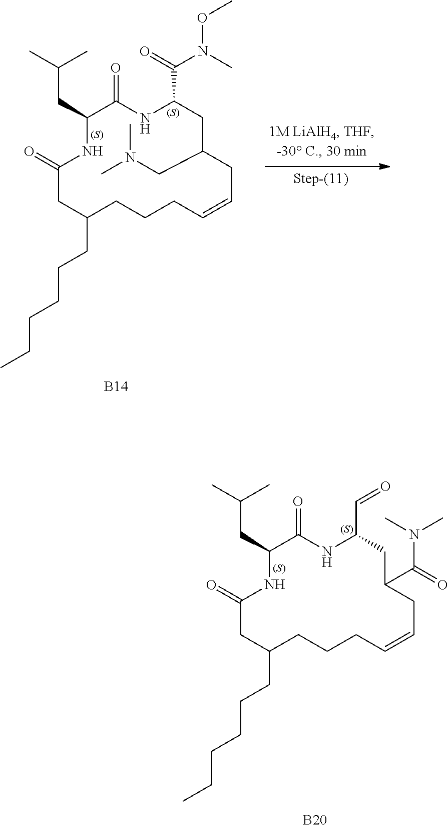

### Methyl 2-(pent-4-en-1-yl)octanoate (3)

To a stirred solution of freshly prepared LDA (n-BuLi 70 mL, 176.94 mmol), DIPA (27 mL, 189.58 mmol in THF (100 mL) at 0° C. was added methyl octanoate (1) (20 g, 126.39 mmol) at RT. Reaction mixture was cooled to −78° C. and stirred for 1 h then added TBAI (2.3 g, 6.32 mmol), HMPA (20 mL) and 5-bromopent-1-ene (2) (24 mL, 202.22 mmol) at same temperature. The reaction mixture was stirred at RT for 16 h. After consumption of starting material reaction mixture was quenched with sat. NH₄Cl solution (150 mL) and extracted with EtOAc (2×150 mL). Combined organic layer was washed with brine (150 mL) then dried over anhydrous Na₂SO₄ and concentrated under reduced pressure to afford crude product; this crude was purified by silica gel (100-200 mesh) chromatography eluting with 100% pet ether to afford Methyl 2-(pent-4-en-1-yl)octanoate (3)

### 2-(pent-4-en-1-yl)octanoic acid (4)

To a stirred solution of methyl 2-(pent-4-en-1-yl)octanoate (3) (12 g, 53.09 mmol) in methanol (50 mL), THE (50 mL), water (10 mL), was added NaOH (8.49 g, 212.38 mmol) at 0° C. and stirred at room temperature for 4 h. Reaction mixture distilled off under reduced pressure, crude compound acidified to pH ˜ using 2N HCl and extracted with EtOAc (2×500 mL), dried over anhydrous Na₂SO₄, concentrated under reduced pressure to afford 2-(pent-4-en-1-yl)octanoic acid (4). This product was used for next step without any purification.

### 2-(pent-4-en-1-yl)octan-1-ol (5)

To a stirred solution of 2-(pent-4-en-1-yl)octanoic acid (4) (8 g, 37.73 mmol) in THE (200 mL) was added 2M LiAlH₄ in THE (94 mL, 188.67 mmol) at −0° C. and reaction mixture was stirred at −0° C. for 3 h, after consumption of starting material, diluted with ice water (100 mL) extracted with EtOAc (2×100 mL), combined organic layer was washed with brine (100 mL), dried over anhydrous Na₂SO₄ and concentrated to afford crude product 2-(pent-4-en-1-yl)octan-1-ol (5).

### 2-(pent-4-en-1-yl)octyl 4-methylbenzenesulfonate (6)

To a stirred solution of 2-(pent-4-en-1-yl)octan-1-ol (5) (3 g, 15.15 mmol) in pyridine (30 mL), was added tosyl chloride (6.35 g, 33.33 mmol), DMAP (4.06 g, 33.33 mmol) at RT and stirred for 16 h. After consumption of starting material, the reaction mixture was quenched with water (200 mL) and extracted with EtOAc (2×200 mL). Combined organic layer was washed with water (100 mL), brine (100 mL) dried over anhydrous Na₂SO₄, evaporated under reduced pressure to afford crude product 2-(pent-4-en-1-yl)octyl 4-methylbenzenesulfonate (6).

### 3-(pent-4-en-1-yl)nonanenitrile (7)

To a stirred solution of 2-(pent-4-en-1-yl)octyl 4-methylbenzenesulfonate (6) (3 g, 8.52 mmol) in DMSO (30 mL), was added NaCN (626 mg, 12.78 mmol) at rt and stirred at 90° C. for 1 h. After consumption of starting material, reaction mixture was quenched with water (100 mL), extracted with ether (2×100 mL), dried over sodium sulphate, evaporated under reduced pressure to afford crude product 3-(pent-4-en-1-yl)nonanenitrile (7).

### 3-(pent-4-en-1-yl)nonanoic acid (8)

To a stirred solution of 3-(pent-4-en-1-yl)nonanenitrile (7) (2 g, 9.66 mmol) in EtOH (20 mL), was added KOH (5.41 mg, 96.62 mmol) at rt and stirred at 100° C. for 16 h. After consumption of starting material, reaction mixture was acidified with 1N HCl, extracted with ether (2×50 mL), dried over anhydrous Na₂SO₄, evaporated under reduced pressure to afford crude product 3-(pent-4-en-1-yl)nonanoic acid (8).

### Ethyl (2S)-4-(dimethylcarbamoyl)-2-((2S)-4-methyl-2-(3-(pent-4-en-1-yl)nonanamido)pentanamido)hept-6-enoate (9)

To a stirred solution of compound-(8) (1.1 g, 4.86 mmol) in DMF (30 mL) at 0° C. was added EDC.HCl (1.39 g, 7.30 mmol), HOBt (985 mg, 7.30 mmol), DIPEA (2.7 mL, 14.60 mmol) and reaction mixture was stirred for 15 min at 0° C. then added ethyl (2S)-2-((S)-2-amino-4-methylpentanamido)-4-(dimethylcarbamoyl)hept-6-enoate (1.9 g, 5.35 mmol) at 0° C. and stirred at RT for 16 h. After consumption of starting material, the reaction mixture was quenched with ice water (100 mL) and extracted with EtOAc (2×50 mL). Combined organic layer was washed with brine (100 mL), dried over anhydrous Na₂SO₄ and concentrated under reduced pressure to afford crude product; this crude product was purified by silica gel (100-200 mesh) column chromatography eluting with 70% EtOAc in hexane to afford Ethyl (2S)-4-(dimethylcarbamoyl)-2-((2S)-4-methyl-2-(3-(pent-4-en-1-yl)nonanamido)pentanamido)hept-6-enoate (9).

### Ethyl (2S,5S,Z)-7-(dimethylcarbamoyl)-14-hexyl-2-isobutyl-3,16-dioxo-1,4-diazacyclohexadec-9-ene-5-carboxylate (10)

To a stirred solution of ethyl (2S)-4-(dimethylcarbamoyl)-2-((2S)-4-methyl-2-(3-(pent-4-en-1-yl)nonanamido)pentanamido)hept-6-enoate (9) (1 g, 1.77 mmol) in DCM (20 mL) was added Grubb's II (150 mg, 0.177 mmol) at rt and stirred at 50° C. for 2 h. After consumption of starting material, reaction mixture was evaporated under reduced pressure to afford crude product; this crude was purified by normal phase chromatography with eluting 4% methanol in dichloromethane to afforded Ethyl (2S,5S,Z)-7-(dimethylcarbamoyl)-14-hexyl-2-isobutyl-3,16-dioxo-1,4-diazacyclohexadec-9-ene-5-carboxylate (10).

### (2S,5S,Z)-7-(dimethylcarbamoyl)-14-hexyl-2-isobutyl-3,16-dioxo-1,4-diazacyclohexadec-9-ene-5-carboxylic acid (11)

To a stirred solution of Ethyl (2S,5S,Z)-7-(dimethylcarbamoyl)-14-hexyl-2-isobutyl-3,16-dioxo-1,4-diazacyclohexadec-9-ene-5-carboxylate (10) (400 mg, 0.74 mmol) in methanol (5 mL) and water (1 mL), was added lithium hydroxide (34 mg, 1.49 mmol) at rt and stirred at room temperature for 3 h. Reaction mixture completely distilled under reduced pressure, crude compound acidified to pH ˜4 using 2N HCl solution and extracted with EtOAc (2×20 mL), dried over anhydrous Na₂SO₄, concentrated under reduced pressure to afford compound (2S,5S,Z)-7-(dimethylcarbamoyl)-14-hexyl-2-isobutyl-3,16-dioxo-1,4-diazacyclohexadec-9-ene-5-carboxylic acid (11).

### (2S,5S,Z)-14-hexyl-2-isobutyl-N5-methoxy-N⁵,N⁷,N⁷-trimethyl-3,16-dioxo-1,4-diazacyclohexadec-9-ene-5,7-dicarboxamide B14

To a stirred solution of (2S,5S,Z)-7-(dimethylcarbamoyl)-14-hexyl-2-isobutyl-3,16-dioxo-1,4-diazacyclohexadec-9-ene-5-carboxylic acid (11) (350 mg, 0.69 mmol) in THE (10 mL) and DCM (10 mL) at 0° C. was added EDC.HCl (158 mg, 0.82 mmol), HOBT (111 mg, 0.82 mmol), N-methylmorpholine (83 mg, 0.82 mmol) and N,O-dimethylhydroxylamine (80 mg, 0.82 mmol) sequentially and stirred at room temperature for 16 h. Reaction mixture was diluted with ice water (20 mL), extracted with EtOAc (2×25 mL), dried over anhydrous Na₂SO₄ and evaporated under reduced pressure. The crude residue was purified by grace RP, using at 60% MeCN in 0.1% formic acid in water to afford (2S,5S,Z)-14-hexyl-2-isobutyl-N5-methoxy-N⁵,N⁷,N⁷-trimethyl-3,16-dioxo-1,4-diazacyclohexadec-9-ene-5,7-dicarboxamide B14.

TLC system: 10% MeOH in DCM; Rf: 0.3

LCMS (ESI): m/z 551.49 [M+H]⁺

### (2S,5S,Z)-5-formyl-14-hexyl-2-isobutyl-N,N-dimethyl-3,16-dioxo-1,4-diazacyclohexadec-9-ene-7-carboxamide B20

To a stirred solution of (2S,5S,Z)-14-hexyl-2-isobutyl-N^{M}-methoxy-N⁵,N⁷,N⁷-trimethyl-3,16-dioxo-1,4-diazacyclohexadec-9-ene-5,7-dicarboxamide B14 (50 mg, 0.09 mmol) in THE (2 mL) was added 1M LiAlH₄ (0.05 mL, 0.09 mmol) at −30° C. and reaction mixture was stirred at −30° C. for 30 min. After consumption of starting material, diluted with ice water (10 mL) extracted with EtOAc (2×10 mL), organic layer was washed with water (2×10 mL), brine (10 mL) and dried over anhydrous Na₂SO₄ and concentrated to afforded (2S,5S,Z)-5-formyl-14-hexyl-2-isobutyl-N,N-dimethyl-3,16-dioxo-1,4-diazacyclohexadec-9-ene-7-carboxamide B20.

TLC system: 10% MeOH in DCM; Rf: 0.1

LCMS (ESI): m/z 492.45 [M+H]⁺

### Synthesis of Compounds B17, B7, B8, and B9

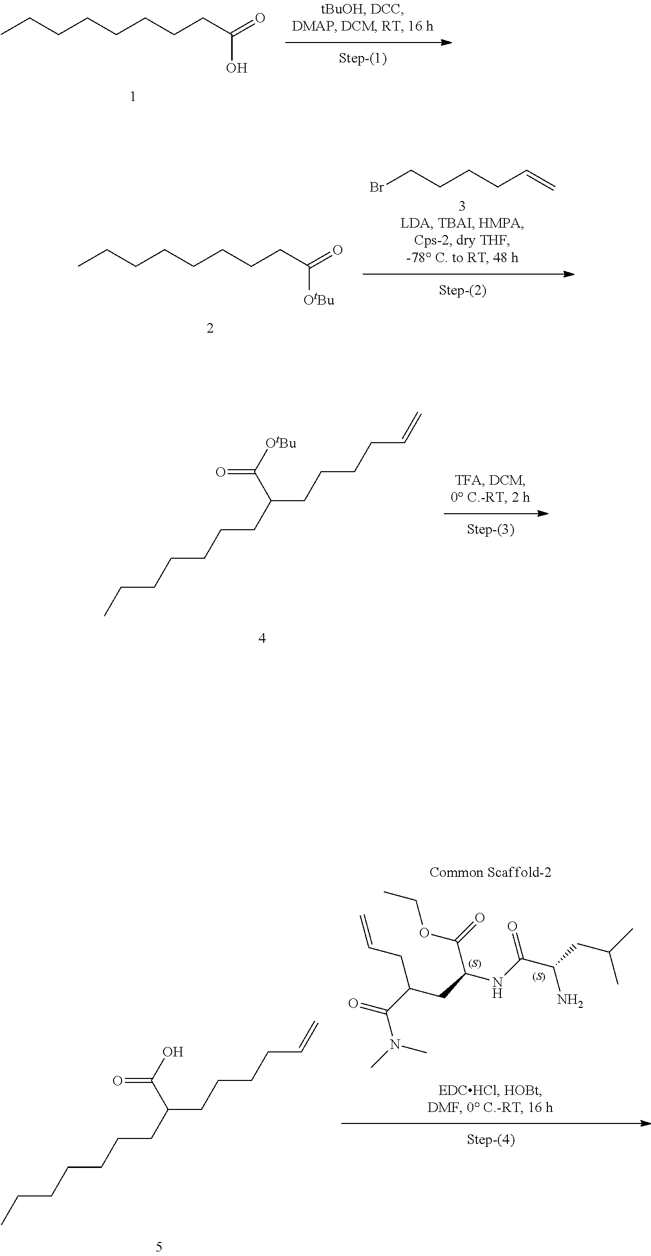

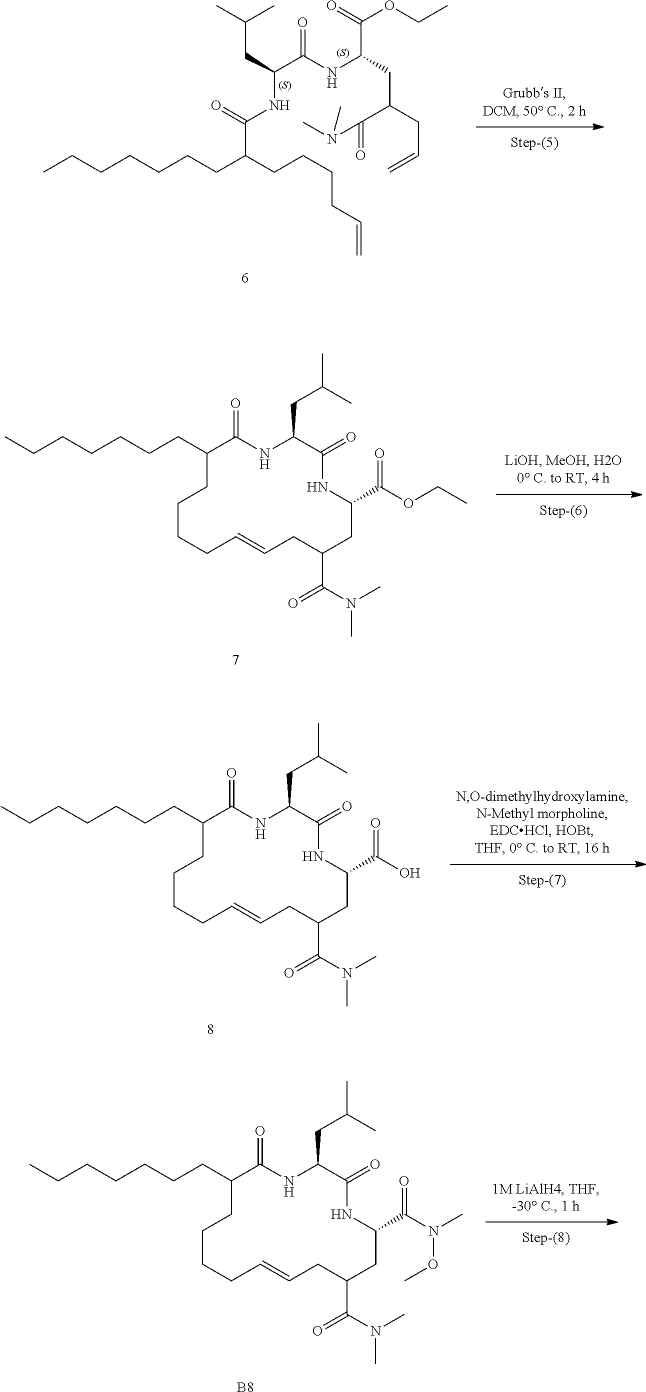

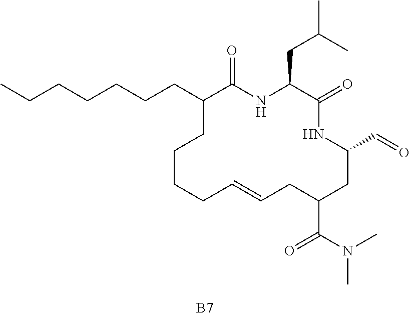 [SMILES: CCCCCCCC1CCCC/C=C/CC(C(=O)N(C)C)C[C@@H](C=O)NC(=O)[C@H](CC(C)C)NC1=O ; "B7"]

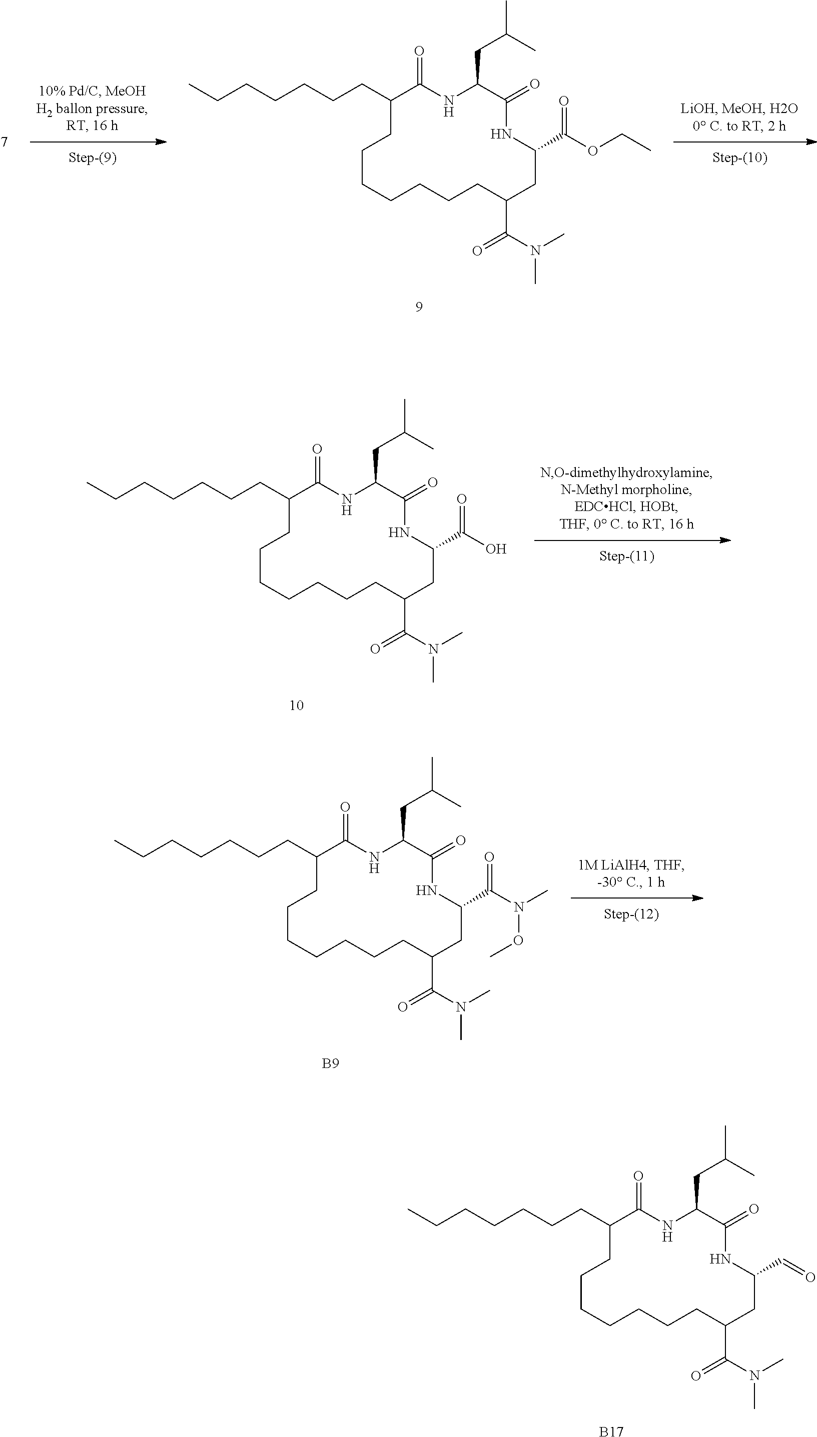

### Tert-butyl nonanoate (2)

To a stirred solution of nonanoic acid (1) (50 g, 0.316 mmol) in DCM (500 mL) was added ^{t}BuOH (46.8 g, 632.9 mmol), DCC (65 g, 316.45 mmol), DMAP (7.7 g, 63.29 mmol) at RT and stirred for 16 h. After consumption of starting material, the reaction mixture was quenched with water (500 mL) and extracted with EtOAc (2×200 mL). Organic layer was washed with water (50 mL), brine (50 mL) dried over anhydrous Na₂SO₄, filtered and evaporated under reduced pressure to afford crude product, this crude was purified by silica (100-200 mesh) column chromatography by eluting with pet ether to afford tert-butyl nonanoate (2).

### tert-butyl 2-(hex-5-en-1-yl)nonanoate (4)

To a stirred solution of freshly prepared LDA (n-BuLi 33 mL, 66.03 mmol), DIPA (10 mL, 70.75 mmol in THE (30 mL) at 0° C. was added tert-butyl nonanoate (2) (10 g, 47.16 mmol) at RT. Reaction mixture was cooled to −78° C. and stirred for 1 h then added TBAI (860 mg, 2.358 mmol), HMPA (10 mL) and 6-bromohex-1-ene (3) (12 g, 75.47 mmol) at same temperature. The reaction mixture was stirred at RT for 16 h. After consumption of starting material reaction mixture was quenched with sat. NH₄Cl solution (150 mL) and extracted with EtOAc (2×50 mL). Organic layer was washed with brine (50 mL) then dried over anhydrous Na₂SO₄ and concentrated under reduced pressure to afford crude product; this crude was purified by silica gel (100-200 mesh) chromatography eluting with 2% EtOAc in hexane to afford tert-butyl 2-(hex-5-en-1-yl)nonanoate (4).

### 2-(hex-5-en-1-yl)nonanoic acid (5)

To a stirred solution of tert-butyl 2-(hex-5-en-1-yl)nonanoate (4) (15 g, 50.67 mmol) in DCM (150 mL) was added TFA (34 mL, 304.05 mmol) at 0° C. and mixture was stirred at RT for 2 h. After consumption of starting material. The solvent was concentrated under reduced pressure to afford crude compound (5) 2-(hex-5-en-1-yl)nonanoic acid (5). This product was used for next step without any purification.

### Ethyl (2S)-4-(dimethylcarbamoyl)-2-((2S)-2-(2-(hex-5-en-1-yl) nonanamido)-4-methylpentanamido) hept-6-enoate (6)

To a stirred solution of 2-(hex-5-en-1-yl)nonanoic acid (5) (3 g, 12.5 mmol) in DMF (30 mL) was added EDC.HCl (3.5 g, 18.75 mmol), HOBt (2.5 g, 18.75 mmol), DIPEA (6.5 mL, 37.5 mmol) at 0° C. and reaction mixture was stirred for 15 min at 0° C. then added ethyl (2S)-2-((S)-2-amino-4-methylpentanamido)-4-(dimethylcarbamoyl)hept-6-enoate (4.4 g, 12.5 mmol) at 0° C. and stirred at RT for 16 h. After consumption of starting material, the reaction mixture was quenched with ice water (100 mL) and extracted with EtOAc (2×50 mL). Organic layer was washed with brine (100 mL), dried over anhydrous Na₂SO₄ and concentrated under reduced pressure to afford crude product; this crude product was purified by silica gel (100-200 mesh) column chromatography eluting with 40% EtAOc in hexane to afford pure compound ethyl (2S)-4-(dimethylcarbamoyl)-2-((2S)-2-(2-(hex-5-en-1-yl)nonanamido)-4-methylpentanamido)hept-6-enoate (6).

### Ethyl (2S,5S,E)-7-(dimethylcarbamoyl)-15-heptyl-2-isobutyl-3,16-dioxo-1, 4-diazacyclohexadec-9-ene-5-carboxylate (7)

To a stirred solution of ethyl (2S)-4-(dimethylcarbamoyl)-2-((2S)-2-(2-(hex-5-en-1-yl)nonanamido)-4-methylpentanamido)hept-6-enoate (6) (1 g, 1.733 mmol) in DCM (100 mL) was added Grubb's II (0.147 g, 0.173 mmol) at rt and stirred at 50° C. for 2 h. After consumption of starting material, reaction mixture was evaporated under reduced pressure to afford crude product; this crude was purified by normal phase chromatography with eluting 4% methanol in dichloromethane to afforded pure ethyl (2S,5S,E)-7-(dimethylcarbamoyl)-15-heptyl-2-isobutyl-3,16-dioxo-1,4-diazacyclohexadec-9-ene-5-carboxylate (7).

### (2S, 5S, E)-7-(dimethylcarbamoyl)-15-heptyl-2-isobutyl-3, 16-dioxo-1, 4-diazacyclohexadec-9-ene-5-carboxylic acid (8)

To a stirred solution of ethyl (2S,5S,E)-7-(dimethylcarbamoyl)-15-heptyl-2-isobutyl-3,16-dioxo-1,4-diazacyclohexadec-9-ene-5-carboxylate (7) (700 mg, 1.275 mmol) in THE (5 mL), methanol (5 mL) and water (2.5 mL), was added lithium hydroxide (0.061 g, 81.30 mmol) at rt and stirred at room temperature for 4 h. Reaction mixture completely distilled under reduced pressure, crude compound acidified with 2N HCL solution up to pH ˜4, and extracted with ethyl acetate (2×100 mL), dried over sodium sulfate, concentrated under reduced pressure to afford compound (2S,5S,E)-7-(dimethylcarbamoyl)-15-heptyl-2-isobutyl-3,16-dioxo-1,4-diazacyclohexadec-9-ene-5-carboxylic acid (8).

### (2S,5S,E)-15-heptyl-2-isobutyl-N⁵-methoxy-N⁵,N⁷,N7-trimethyl-3,16-dioxo-1,4-diazacyclohexadec-9-ene-5,7-dicarboxamide B8

To a stirred solution of (2S,5S,E)-7-(dimethylcarbamoyl)-15-heptyl-2-isobutyl-3,16-dioxo-1,4-diazacyclohexadec-9-ene-5-carboxylic acid (8) (700 mg, 1.343 mmol) in tetrahydrofuran (3.5 mL) and DCM (3.5 mL) added EDC.HCl (307 mg, 1.612 mmol), HOBT (219 mg, 1.612 mmol), N-methylmorpholine (162 mg, 1.612 mmol) and N,O-dimethylhydroxylamine (144 mg, 1.612 mmol) at 0° C. simultaneously and stirred at room temperature for 16 h. Reaction mixture was diluted with ice water (50 mL), extracted with ethyl acetate (2×25 mL), dried over sodium sulfate and evaporated under reduced pressure. The crude residue was purified by prep-HPLC to afford (2S,5S,E)-15-heptyl-2-isobutyl-N⁵-methoxy-N⁵,N⁷,N⁷-trimethyl-3,16-dioxo-1,4-diazacyclohexadec-9-ene-5,7-dicarboxamide B8.

TLC system: 10% Methanol in DCM; Rf: 0.1

LCMS (ESI): m/z 565.38 [M+H]⁺

### (2S, 5S, E)-5-formyl-15-heptyl-2-isobutyl-N, N-dimethyl-3, 16-dioxo-1, 4-diazacyclohexadec-9-ene-7-carboxamide B7

To a stirred solution of (2S,5S,E)-15-heptyl-2-isobutyl-N⁵-methoxy-N⁵,N⁷,N⁷-trimethyl-3,16-dioxo-1,4-diazacyclohexadec-9-ene-5,7-dicarboxamide B8 (100 mg, 0.177 mmol) in THE (5 mL) was added 1M LiAlH₄ (0.17 mL, 0.177 mmol) at −30° C. and reaction mixture was stirred at −30° C. for 1 h. After consumption of starting material, diluted with ice water (50 mL) extracted with ethyl acetate (2×25 mL), organic layer was washed with water (2×20 mL), and brine (20 mL). Organic layer was dried over anhydrous sodium sulfate and concentrated to afford crude product; this crude was purified by prep HPLC to afforded (2S,5S,E)-5-formyl-15-heptyl-2-isobutyl-N,N-dimethyl-3,16-dioxo-1,4-diazacyclohexadec-9-ene-7-carboxamide B7.

TLC system: 10% Methanol in DCM; Rf: 0.1

LCMS (ESI): m/z 506.35 [M+H]⁺

### ethyl (2S,5S)-7-(dimethylcarbamoyl)-15-heptyl-2-isobutyl-3,16-dioxo-1,4-diazacyclohexadecane-5-carboxylate

To a stirred solution of ethyl (2S,5S,E)-7-(dimethylcarbamoyl)-15-heptyl-2-isobutyl-3,16-dioxo-1,4-diazacyclohexadec-9-ene-5-carboxylate (7) (1 g, 1.82 mmol) in MeOH (10 mL) added 10% Pd/C (100 mg) and stirred at H₂ atmosphere for 16 h. After consumption of starting material, reaction mixture was filtered through celite bed and washed with MeOH (100 mL) the filtrate mL's was evaporated under reduced pressure to afford crude ethyl (2S,5S)-7-(dimethylcarbamoyl)-15-heptyl-2-isobutyl-3,16-dioxo-1,4-diazacyclohexadecane-5-carboxylate (9).

### (2S,5S)-7-(dimethylcarbamoyl)-15-heptyl-2-isobutyl-3,16-dioxo-1,4-diazacyclohexadecane-5-carboxylic acid (10)

To a stirred solution of ethyl (2S,5S)-7-(dimethylcarbamoyl)-15-heptyl-2-isobutyl-3,16-dioxo-1,4-diazacyclohexadecane-5-carboxylate (9) (800 mg, 0.202 mmol) in THE (3 mL), methanol (3 mL) and water (2 mL), was added lithium hydroxide (0.104 g, 4.355 mmol) at rt and stirred at room temperature for 4 h. Reaction mixture completely distilled under reduced pressure, crude compound acidified with 2N HCL solution up to pH ˜4, and extracted with EtOAc (2×100 mL), dried over Na₂SO₄, concentrated under reduced pressure to afford compound (2S,5S)-7-(dimethylcarbamoyl)-15-heptyl-2-isobutyl-3,16-dioxo-1,4-diazacyclohexadecane-5-carboxylic acid 10.

### (2S,5S)-15-heptyl-2-isobutyl-N⁵-methoxy-N⁵,N⁷,N⁷-trimethyl-3,16-dioxo-1,4-diazacyclohexadecane-5,7-dicarboxamide B9

To a stirred solution of (2S,5S)-7-(dimethylcarbamoyl)-15-heptyl-2-isobutyl-3,16-dioxo-1,4-diazacyclohexadecane-5-carboxylic acid (10) (700 mg, 1.338 mmol) in THE (3.5 mL) and DCM (3.5 mL) at 0° C. was added EDC.HCl (306 mg, 1.606 mmol), HOBT (218 mg, 1.606 mmol), N-methylmorpholine (162 mg, 1.606 mmol) and N,O-dimethylhydroxylamine (156 mg, 1.60 mmol) then stirred at room temperature for 16 h. Reaction mixture was diluted with ice water (50 mL), extracted with EtOAc (2×25 mL), dried over Na₂SO₄ and evaporated under reduced pressure. The crude residue was purified by prep HPLC to afford (2S,5S)-15-heptyl-2-isobutyl-N⁵-methoxy-N⁵,N⁷,N⁷-trimethyl-3,16-dioxo-1,4-diazacyclohexadecane-5,7-dicarboxamide B9.

TLC system: 10% Methanol in DCM; Rf: 0.1

LCMS (ESI): m/z 567.5 [M+H]⁺

### ((2S,5S)-5-formyl-15-heptyl-2-isobutyl-N,N-dimethyl-3,16-dioxo-1,4-diazacyclohexadecane-7-carboxamide B17

To a stirred solution of (2S,5S)-15-heptyl-2-isobutyl-N⁵-methoxy-N⁵,N⁷,N⁷-trimethyl-3,16-dioxo-1,4-diazacyclohexadecane-5,7-dicarboxamide B9 (200 mg, 0.35 mmol) in THE (5 mL) was added 1M LiAlH₄ (0.3 mL, 0.35 mmol) at −30° C. and reaction mixture was stirred at −30° C. for 1 h. After consumption of starting material, diluted with ice water (50 mL) extracted with EtOAc (2×25 mL), organic layer was washed with water (2×20 mL), brine (20 mL) and dried over anhydrous Na₂SO₄ and concentrated to afford crude product; this crude was purified by prep HPLC to afforded (2S,5S)-5-formyl-15-heptyl-2-isobutyl-N,N-dimethyl-3,16-dioxo-1,4-diazacyclohexadecane-7-carboxamide B17.

TLC system: 10% Methanol in DCM; Rf: 0.1

LCMS (ESI): m/z 508.48 [M+H]⁺

### Synthesis of Compounds B19 and B18

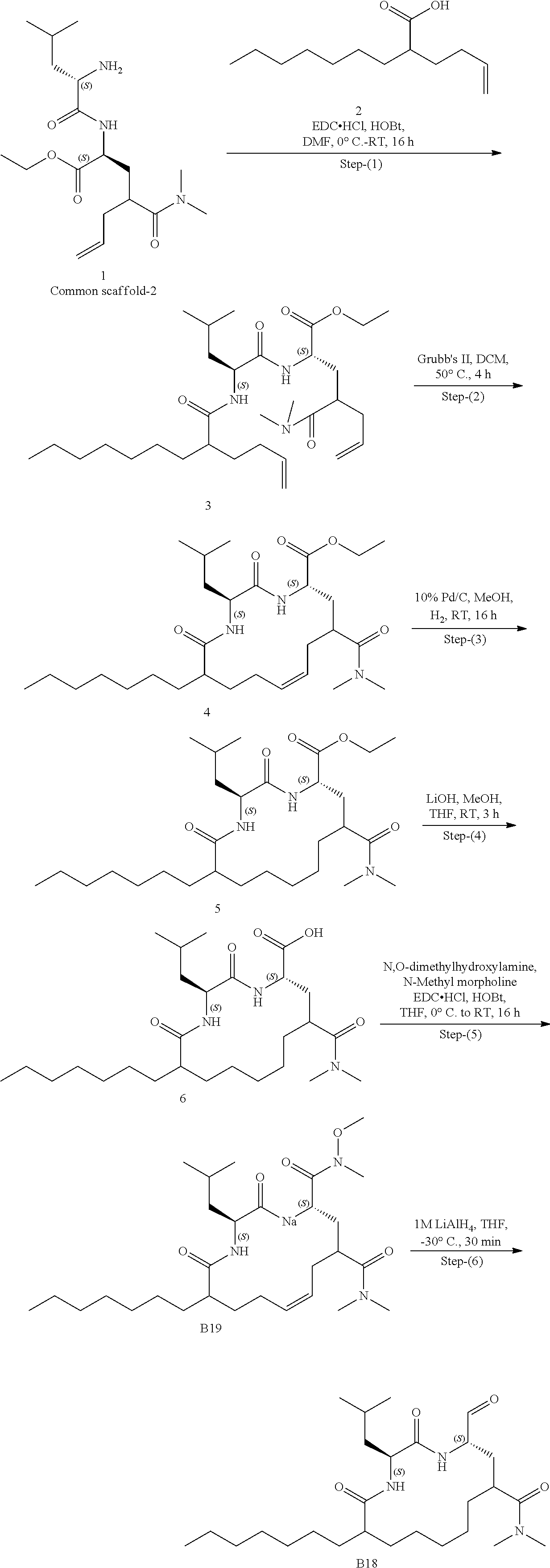

### Ethyl (2S)-2-((2S)-2-(2-(but-3-en-1-yl)nonanamido)-4-methylpentanamido)-4-(dimethylcarbamoyl)hept-6-enoate (3)

To a stirred solution of 2-(but-3-en-1-yl)nonanoic acid (2) (2 g, 9.43 mmol) in DMF (30 mL) at 0° C. was added EDC.HCl (2.72 g, 14.15 mmol), HOBt (1.91 g, 14.15 mmol), DIPEA (5.2 mL, 28.30 mmol) and reaction mixture was stirred for 15 min then added Ethyl (2S)-2-((S)-2-amino-4-methylpentanamido)-4-(dimethylcarbamoyl)hept-6-enoate (3.68 g, 10.37 mmol) at 0° C. and stirred at RT for 16 h. After consumption of starting material, the reaction mixture was quenched with ice water (100 mL) and extracted with EtOAc (2×50 mL). Organic layer was washed with brine (100 mL), dried over anhydrous Na₂SO₄ and concentrated under reduced pressure to afford crude product. It was purified by silica gel (100-200 mesh) column chromatography using 60% EtAOc in hexane as eluent to afford pure compound Ethyl (2S)-2-((2S)-2-(2-(but-3-en-1-yl)nonanamido)-4-methylpentanamido)-4-(dimethylcarbamoyl)hept-6-enoate (3).

### Ethyl (2S,5S,Z)-7-(dimethylcarbamoyl)-13-heptyl-2-isobutyl-3,14-dioxo-1,4-diazacyclotetradec-9-ene-5-carboxylate (4)

To a stirred solution of ethyl (2S)-2-((2S)-2-(2-(but-3-en-1-yl)nonanamido)-4-methylpentanamido)-4-(dimethylcarbamoyl)hept-6-enoate (3) (1 g, 1.82 mmol) in DCM (100 mL) was added Grubb's II (0.154 g, 0.18 mmol) at rt and stirred at 50° C. for 4 h. After consumption of starting material, reaction mixture was evaporated under reduced pressure to afford crude residue. It was purified by normal phase chromatography with eluting 4% methanol in dichloromethane to afforded Ethyl (2S,5S,Z)-7-(dimethylcarbamoyl)-13-heptyl-2-isobutyl-3,14-dioxo-1,4-diazacyclotetradec-9-ene-5-carboxylate (4).

### Methyl (2S,5S)-7-(dimethylcarbamoyl)-13-heptyl-2-isobutyl-3,14-dioxo-1,4-diazacyclotetradecane-5-carboxylate (5)

To a stirred solution of ethyl (2S,5S,Z)-7-(dimethylcarbamoyl)-13-heptyl-2-isobutyl-3,14-dioxo-1,4-diazacyclotetradec-9-ene-5-carboxylate (4) (300 mg, 0.57 mmol) in MeOH (5 mL) added 10% Pd/C (150 mg) and stirred at H₂ atmosphere for 16 h. After consumption of starting material, reaction mixture was filtered through celite bed and washed with MeOH (50 mL) the filtrate mL's was evaporated under reduced pressure to afford crude methyl (2S,5S)-7-(dimethylcarbamoyl)-13-heptyl-2-isobutyl-3,14-dioxo-1, 4-diazacyclotetradecane-5-carboxylate (5).

### (2S,5S)-7-(dimethylcarbamoyl)-13-heptyl-2-isobutyl-3,14-dioxo-1,4-diazacyclotetradecane-5-carboxylic acid (6)

To a stirred solution of ethyl (2S,5S)-7-(dimethylcarbamoyl)-13-heptyl-2-isobutyl-3,14-dioxo-1,4-diazacyclotetradecane-5-carboxylate (5) (300 mg, 0.57 mmol) in MeOH (5 mL) and H₂O (1 mL), was added LiOH (0.027 g, 1.14 mmol) at rt and stirred at room temperature for 2 h. Reaction mixture was distilled under reduced pressure, crude compound acidified to pH ˜4 with 2N HCl and extracted with EtOAc (2×10 mL), dried over anhydrous Na₂SO₄, evaporated under reduced pressure to afford (2S,5S)-7-(dimethylcarbamoyl)-13-heptyl-2-isobutyl-3,14-dioxo-1,4-diazacyclotetradecane-5-carboxylic acid (6).

### (2S,5S)-13-heptyl-2-isobutyl-N⁵-methoxy-N⁵,N⁷,N⁷-trimethyl-3,14-dioxo-1,4-diazacyclotetradecane-5,7-dicarboxamide B19

To a stirred solution of (2S,5S)-7-(dimethylcarbamoyl)-13-heptyl-2-isobutyl-3,14-dioxo-1,4-diazacyclotetradecane-5-carboxylic acid (6) (250 mg, 0.50 mmol) in THE (5 mL) and DCM (5 mL) added EDC.HCl (116 mg, 0.61 mmol), HOBT (82 mg, 0.61 mmol), N-methylmorpholine (61 mg, 0.61 mmol) and N,O-dimethylhydroxylamine (58 mg, 0.61 mmol) at 0° C. simultaneously and stirred at room temperature for 16 h. Reaction mixture was diluted with ice water (20 mL), extracted with EtOAc (2×20 mL), dried over anhydrous Na₂SO₄ and evaporated under reduced pressure. The crude residue was purified by grace RP by using 60% MeCN in 0.1% formic acid in water, to afford (2S,5S)-13-heptyl-2-isobutyl-N5-methoxy-N⁵,N⁷,N⁷-trimethyl-3,14-dioxo-1,4-diazacyclotetradecane-5,7-dicarboxamide B19

TLC system: 10% MeOH in DCM; Rf: 0.3

LCMS (ESI): m/z 539.49 [M+H]⁺

### (2S,5S)-5-formyl-13-heptyl-2-isobutyl-N,N-dimethyl-3,14-dioxo-1,4-diazacyclotetradecane-7-carboxamide B18

To a stirred solution of (2S,5S)-13-heptyl-2-isobutyl-N⁵-methoxy-N⁵,N⁷,N⁷-trimethyl-3,14-dioxo-1,4-diazacyclotetradecane-5,7-dicarboxamide B19 (50 mg, 0.09 mmol) in THE (5 mL) was added 2M LiAlH₄ (0.05 mL, 0.09 mmol) at −30° C. and reaction mixture was stirred at −30° C. for 30 min. After consumption of starting material, diluted with ice water (5 mL) extracted with EtOAc (2×5 mL), combined organic layer was washed with water (2×5 mL), and brine (5 mL) and dried over anhydrous Na₂SO₄ and concentrated to afford (2S,5S)-5-formyl-13-heptyl-2-isobutyl-N,N-dimethyl-3,14-dioxo-1,4-diazacyclotetradecane-7-carboxamide B18.

TLC system: 10% MeOH in DCM; Rf: 0.2

LCMS (ESI): m/z 480.41 [M+H]⁺

### Synthesis of Compound B22 and B68

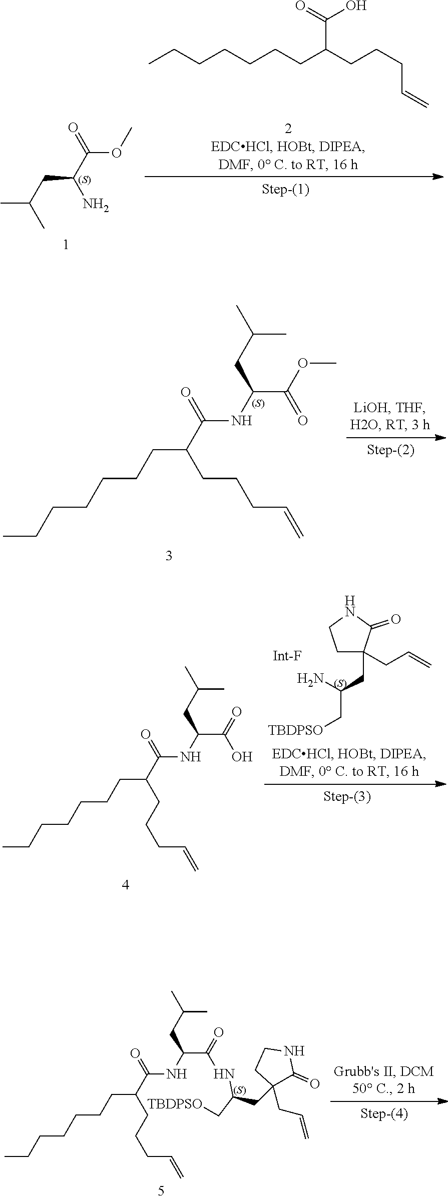

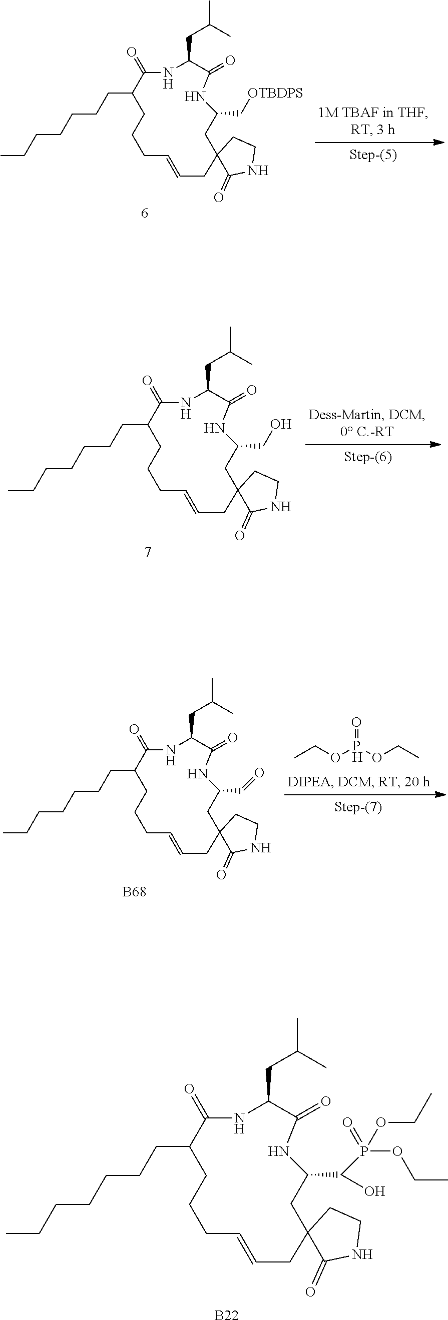

### Methyl (2-(pent-4-en-1-yl) nonanoyl)-L-leucinate (3)

To a stirred solution of 2-(pent-4-en-1-yl)nonanoic acid (2) (3.5 g, 15.48 mmol) in DMF (40 mL) at 0° C. was added EDC.HCl (3.4 g, 18.58 mmol), HOBt (2.4 g, 18.58 mmol), DIPEA (9.9 mL, 46.44 mmol) and reaction mixture was stirred for 15 min. at 0° C. then added methyl L-leucinate (3.62 g, 18.5 mmol) at 0° C. and stirred at RT for 16 h. After consumption of starting material, the reaction mixture was quenched with ice water (100 mL) and extracted with EtOAc (2×50 mL). Organic layer was washed with brine (100 mL), dried over anhydrous Na₂SO₄ and concentrated under reduced pressure to afford crude product; this crude product was purified by silica gel (100-200 mesh) column chromatography eluting with 5% EtAOc in hexane to afford methyl (2-(pent-4-en-1-yl)nonanoyl)-L-leucinate (3).

### (2-(pent-4-en-1-yl) nonanoyl)-L-leucine (4)

To a stirred solution of methyl (2-(pent-4-en-1-yl)nonanoyl)-L-leucinate (3) (2 g, 5.66 mmol) in THE (30 mL), water (10 mL) was added LiOH (0.27 g, 11.33 mmol) at rt and stirred at room temperature for 3 h. Reaction mixture was distilled off under reduced pressure, crude residue was acidified to pH ˜4 using 2N HCl solution up and extracted with EtOAc (2×50 mL), dried over anhydrous sodium sulfate, concentrated under reduced pressure to afford compound (2-(pent-4-en-1-yl)nonanoyl)-L-leucine (4).

### N-((2S)-1-(((2S)-1-(3-allyl-2-oxopyrrolidin-3-yl)-3-((tert-butyldiphenylsilyl) oxy) propan-2-yl) amino)-4-methyl-1-oxopentan-2-yl)-2-(pent-4-en-1-yl) nonanamide (5)

To a stirred solution of (2-(pent-4-en-1-yl)nonanoyl)-L-leucine (4) (388 mg, 1.14 mmol) in DMF (10 mL) at 0° C. was added EDC.HCl (328 mg, 1.71 mmol), HOBt (232 mg, 1.71 mmol), DIPEA (0.6 mL, 3.43 mmol) and reaction mixture was stirred for 15 min at 0° C. then added 3-allyl-3-((S)-2-amino-3-((tert-butyldiphenylsilyl)oxy)propyl)pyrrolidin-2-one (500 mg, 1.14 mmol) at 0° C. and stirred at RT for 16 h. After consumption of starting material, the reaction mixture was quenched with ice water (50 mL) and extracted with EtOAc (2×40 mL). Organic layer was washed with brine (100 mL), dried over anhydrous Na₂SO₄ and concentrated under reduced pressure to afford crude product; this crude product was purified by normal chromatography eluting with 5% MeOH in dichloromethane to afford N-((2S)-1-(((2S)-1-(3-allyl-2-oxopyrrolidin-3-yl)-3-((tert-butyldiphenylsilyl)oxy)propan-2-yl)amino)-4-methyl-1-oxopentan-2-yl)-2-(pent-4-en-1-yl)nonanamide (5).

### (7S,10S,E)-7-(((tert-butyldiphenylsilyl) oxy) methyl)-13-heptyl-10-isobutyl-2, 8, 11-triazaspiro [4.14] nonadec-17-ene-1, 9, 12-trione (6)

To a stirred solution of N-((2S)-1-(((2S)-1-(3-allyl-2-oxopyrrolidin-3-yl)-3-((tert-butyldiphenylsilyl)oxy)propan-2-yl)amino)-4-methyl-1-oxopentan-2-yl)-2-(pent-4-en-1-yl)nonanamide (5) (550 mg, 0.72 mmol) in DCM (50 mL) was added Grubb's II (61 mg, 0.1 mmol) at room temperature and stirred at 50° C. for 2 h. After consumption of starting material, reaction mixture was evaporated under reduced pressure to afford crude product; this crude was purified by normal phase chromatography with eluting 4% methanol in dichloromethane to afforded pure (7S,10S,E)-7-(((tert-butyldiphenylsilyl)oxy)methyl)-13-heptyl-10-isobutyl-2,8,11-triazaspiro[4.14]nonadec-17-ene-1,9,12-trione (6).

### (7S, 10S, E)-13-heptyl-7-(hydroxymethyl)-10-isobutyl-2, 8, 11-triazaspiro [4.14] nonadec-17-ene-1, 9, 12-trione (7)

To a stirred solution of (7S,10S,E)-7-(((tert-butyldiphenylsilyl)oxy)methyl)-13-heptyl-10-isobutyl-2,8,11-triazaspiro[4.14]nonadec-17-ene-1,9,12-trione (6) (300 mg, 0.41 mmol) in THE (5 mL) was added 1M TBAF in THE (0.8 mL, 0.82 mmol) at rt and stirred at room temperature for 3 h. Reaction mixture was distilled off under reduced pressure, crude compound diluted with water (20 mL) and extracted with EtOAc (2×50 mL), dried over anhydrous sodium sulfate, concentrated under reduced pressure to afford crude product. It was purified by normal phase chromatography with eluting 8% MeOH in DCM to afford (7S,10S,E)-13-heptyl-7-(hydroxymethyl)-10-isobutyl-2,8,11-triazaspiro[4.14]nonadec-17-ene-1,9,12-trione.

### (7S, 10S, E)-13-heptyl-10-isobutyl-1, 9, 12-trioxo-2, 8, 11-triazaspiro [4.14] nonadec-17-ene-7-carbaldehyde B68

To a stirred solution of (7S,10S,E)-13-heptyl-7-(hydroxymethyl)-10-isobutyl-2,8,11-triazaspiro[4.14]nonadec-17-ene-1,9,12-trione (7) (150 mg, 0.305 mmol) was dissolved in DCM (10 mL) at 0° C. was added Dess-Martin periodinane (2583 mg, 1.8 mmol) followed by NaHCO₃ (77 mg, 0.91 mmol) and stirred at RT for 3 h. Reaction mixture was taken in DCM (30 mL) and washed with sat. Hypo solution (3×20 mL), sat. NaHCO₃ solution (3×20 mL). Organic layer was dried over anhydrous Na₂SO₄, filtered and concentrated to get crude (7S, 10S, E)-13-heptyl-10-isobutyl-1,9,12-trioxo-2,8,11-triazaspiro[4.14]nonadec-17-ene-7-carbaldehyde B68 It was used into next step without any purification.

TLC system: 10% MeOH in DCM Rf: 0.3

LCMS (ESI): m/z 490.48 [M+H]⁺

### Diethyl ((1R)-((7S,10S,E)-13-heptyl-10-isobutyl-1,9,12-trioxo-2,8,11-triazaspiro[4.14]nonadec-17-en-7-yl)(hydroxy)methyl)phosphonate B22

To a stirred solution of (7S,10S,E)-13-hexyl-10-isobutyl-1,9,12-trioxo-2,8,11-triazaspiro[4.14]nonadec-17-ene-7-carbaldehyde (B68) (120 mg, 0.24 mmol) in DCM (3 mL) was added DIPEA (0.12 mL, 0.73 mmol) and diethyl phosphite (0.14 mL, 0.73 mmol). Then reaction mixture was stirred at RT for 16 h (monitoring by TLC indicated complete disappearance of the starting material) and reaction mixture was taken in DCM (20 mL) and washed with water (2×30 mL) and brine solution. Organic layer was dried over anhydrous Na₂SO₄, and evaporated to afford crude residue. It was purified by prep HPLC and afforded diethyl ((1R)-((7S,10S,E)-13-heptyl-10-isobutyl-1,9,12-trioxo-2,8,11-triazaspiro[4.14]nonadec-17-en-7-yl)(hydroxy)methyl)phosphonate as formic acid salt B22.

TLC system: 10% MeOH in DCM; Rf: 0.1

LCMS (ESI): m/z 628.60[M+H]⁺

### Synthesis of Compounds B23 and B69

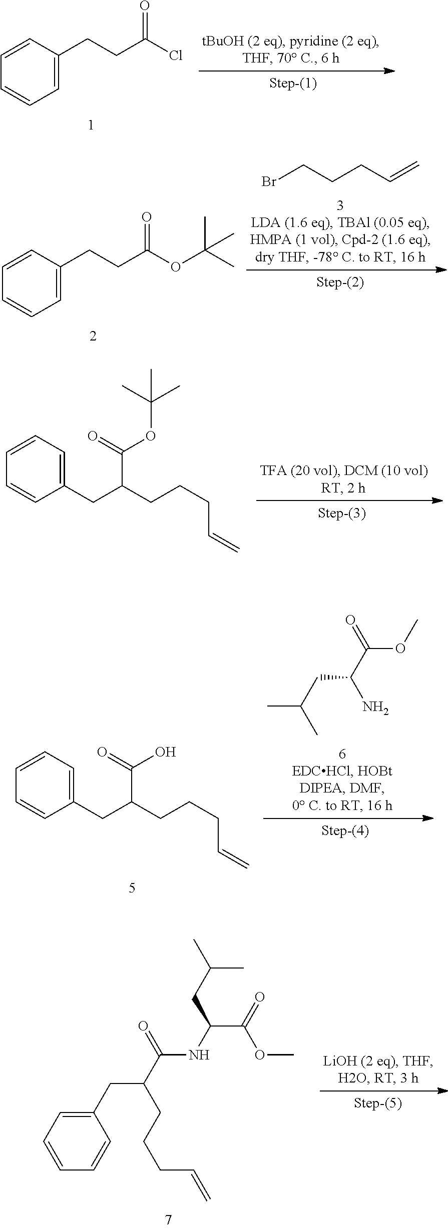

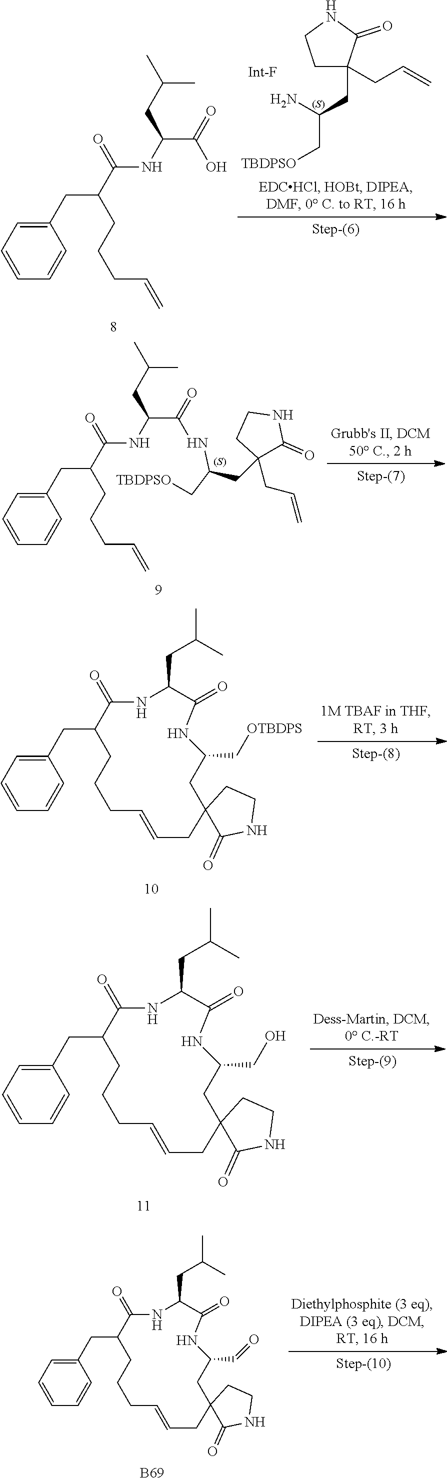

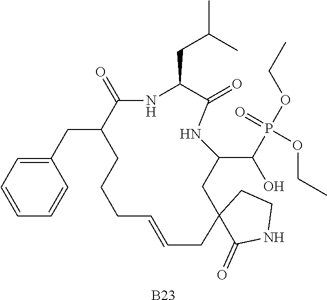

### Tert-butyl 3-phenylpropanoate (2)

To a stirred solution of 3-phenylpropanoyl chloride (1) (10 g, 59.5238 mmol) in THF (100 mL) cooled to 0° C. and added pyridine (9.4 mL, 119.047 mmol) and followed by t-BuOH (4.4 mL, 119.047 mmol) and then reaction mixture was stirred at 70° C. for 6 h. The progress of the reaction was monitored by TLC and LCMS. After 6 h, the reaction mixture quenched with water (30 mL) and extracted with ethyl acetate (2×100 mL). Combined organic layer was washed with brine solution, dried over Na₂SO₄ and concentrated to get crude. Crude compound was purified by column chromatography to afford pure compound tert-butyl 3-phenylpropanoate 2.

### Tert-butyl 2-benzylhept-6-enoate (4)

To a stirred solution of tert-butyl 3-phenylpropanoate (5 g, 24.2718 mmol) in THE (50 mL), was added 2M LDA (19.4 mL, 38.834 mmol). Reaction mixture was cooled to −78° C. and stirred for 1 h then added TBAI (447 mg, 1.2135 mmol), HMPA (5 mL) and 5-bromopent-1-ene (3) (3.6 mL, 11.65 mmol) at same temperature. The reaction mixture was stirred at RT for 16 h. The progress of the reaction was monitored by TLC and LCMS. After 16 h, the reaction mixture was quenched with sat. NH₄Cl solution (100 mL) and extracted with EtOAc (2×50 mL). Combined organic layer was washed with brine (50 mL) then dried over sodium sulfate and concentrated under reduced pressure to afford crude product. The crude product was purified by column chromatography by using silica-gel (100-200 mesh) by eluting 2% ethyl acetate in hexane to afford tert-butyl 2-benzylhept-6-enoate (4).

### 2-benzylhept-6-enoic acid (5)

To a stirred solution of tert-butyl 2-benzylhept-6-enoate (4) (3 g, 10.948 mmol) in DCM (30 mL) was added TFA (15 mL) at 0° C. and the reaction mixture was stirred at RT for 3 h. The progress of the reaction was monitored by TLC and LCMS. After 3 h, the solvents were concentrated under reduced pressure to afford crude compound 2-benzylhept-6-enoic acid (5). This product was used for next step without any purification. Methyl (2-benzylhept-6-enoyl)-L-leucinate (7)

To a stirred solution of 2-benzylhept-6-enoic acid (5) (1.5 g, 6.8807 mmol) in DMF (20 mL) was added EDC.HCl (1.97 g, 10.3211 mmol), HOBt (1.4 g, 10.3211 mmol), DIPEA (3.77 mL, 46.44 mmol) at 0° C. and reaction mixture was stirred for 15 min at 0° C. then added methyl D-leucinate hydrochloride (6) (1.5 g, 8.2568 mmol) at 0° C. and stirred at RT for 16 h. The reaction mixture was stirred at RT for 16 h. The progress of the reaction was monitored by TLC and LCMS. After 16 h, the reaction mixture was quenched with ice water (100 mL) and extracted with EtOAc (2×50 mL). The combined organic layer was washed with brine (100 mL), dried over anhydrous Na₂SO₄ and concentrated under reduced pressure to afford crude product; this crude product was purified by silica gel (100-200 mesh) column chromatography eluting with 5% EtOAc in hexane to afford methyl (2-benzylhept-6-enoyl)-L-leucinate (7).

### (2-Benzylhept-6-enoyl)-L-leucine (8)

To a stirred solution of methyl (2-benzylhept-6-enoyl)-L-leucinate (7) (1.5 g, 4.3478 mmol) in THE (20 mL), water (10 mL) was added lithium hydroxide (313 mg, 13.0434 mmol) at RT and continued to stir at RT for 3 h. The progress of the reaction was monitored by TLC and LCMS. After 3 h, the reaction mixture completely distilled under reduced pressure, crude compound acidified with 2N HCl solution up to pH ˜2, and extracted with ethyl acetate (2×50 mL), dried over sodium sulfate, concentrated under reduced pressure to afford compound (2-benzylhept-6-enoyl)-L-leucine (8).

### N-((2S)-1-(((2S)-1-(3-Allyl-2-oxopyrrolidin-3-yl)-3-((tert-butyldiphenylsilyl)oxy)propan-2-yl)amino)-4-methyl-1-oxopentan-2-yl)-2-benzylhept-6-enamide (9)

To a stirred solution of (2-benzylhept-6-enoyl)-L-leucine (8) (1 g, 3.0211 mmol) in DMF (20 mL) was added EDC.HCl (865 mg, 4.5317 mmol), HOBt (611 mg, 4.5317 mmol), DIPEA (1.67 mL, 9.0634 mmol) at 0° C. and reaction mixture was stirred for 15 min at 0° C. then added 3-allyl-3-((S)-2-amino-3-((tert-butyldiphenylsilyl)oxy)propyl)pyrrolidin-2-one (1.4 g, 3.3232 mmol) at 0° C. and stirred at RT for 16 h. The progress of the reaction was monitored by TLC and LCMS. Then reaction mixture was quenched with ice water (50 mL) and extracted with EtOAc (2×50 mL). The combined organic layer was washed with brine (100 mL), dried over anhydrous Na₂SO₄ and concentrated under reduced pressure to afford crude product; this crude product was purified by normal chromatography eluting with 30% hexane in ethyl acetate to afford N-((2S)-1-(((2S)-1-(3-allyl-2-oxopyrrolidin-3-yl)-3-((tert-butyldiphenylsilyl)oxy)propan-2-yl)amino)-4-methyl-1-oxopentan-2-yl)-2-benzylhept-6-enamide (9).

### (7S, 10S)-13-Benzyl-7-(((tert-butyldiphenylsilyl)oxy)methyl)-10-isobutyl-2,8,11-triazaspiro[4.14]nonadec-17-ene-1,9,12-trione (10)

To a stirred solution of N-((2S)-1-(((2S)-1-(3-allyl-2-oxopyrrolidin-3-yl)-3-((tert-butyldiphenylsilyl)oxy)propan-2-yl)amino)-4-methyl-1-oxopentan-2-yl)-2-benzylhept-6-enamide (9) (2×600 mg, 0.801 mmol) in DCM (40 mL) was added Grubb's II catalyst (68 mg, 0.08 mmol) at RT and stirred at 50° C. for 2 h. The progress of the reaction was monitored by TLC and LCMS. After 2 h, reaction mixture was evaporated under reduced pressure to afford crude product; this crude was purified by normal phase chromatography with eluting 4% methanol in dichloromethane to afford (7S,10S)-13-benzyl-7-(((tert-butyldiphenylsilyl)oxy)methyl)-10-isobutyl-2,8,11-triazaspiro[4.14]nonadec-17-ene-1,9,12-trione (10).

### (7S,10S)-13-Benzyl-7-(hydroxymethyl)-10-isobutyl-2,8,11-triazaspiro[4.14]nonadec-17-ene-1,9,12-trione (11)

To a stirred solution of (7S,10S)-13-benzyl-7-(((tert-butyldiphenylsilyl)oxy)methyl)-10-isobutyl-2,8,11-triazaspiro[4.14]nonadec-17-ene-1,9,12-trione (10) (800 mg, 1.109 mmol) in THE (20 mL) was added 1M TBAF in THE (1.16 mL, 1.166 mmol) at RT and stirred at room temperature for 3 h. The progress of the reaction was monitored by TLC and LCMS. After 3 h, the reaction mixture completely distilled under reduced pressure, crude compound diluted with water and extracted with ethyl acetate (2×80 mL), dried over sodium sulfate, concentrated under reduced pressure to afford crude product; this crude was purified by normal phase chromatography with eluting 8% methanol in dichloromethane to afford (7S,10S)-13-benzyl-7-(hydroxymethyl)-10-isobutyl-2,8,11-triazaspiro[4.14]nonadec-17-ene-1,9,12-trione (11).

### (7S, 10S)-13-Benzyl-10-isobutyl-1, 9, 12-trioxo-2, 8, 11-triazaspiro [4.14] nonadec-17-ene-7-carbaldehyde B69

To a stirred solution of (7S, 10S)-13-benzyl-7-(hydroxymethyl)-10-isobutyl-2,8,11-triazaspiro[4.14]nonadec-17-ene-1,9,12-trione (11) (180 mg, 0.305 mmol) in DCM (15 mL) at 0° C. was added Dess-Martin periodinane (237 mg, 0.559 mmol) followed by NaHCO₃ (93 mg, 1.118 mmol) and stirred at RT for 3 h. The progress of the reaction was monitored by TLC and LCMS. After 3 h, the reaction mixture was diluted by DCM (30 mL) and washed with sat. Hypo solution (3×20 mL), sat. NaHCO₃ solution (3×20 mL). Organic layer was dried over anhydrous Na₂SO₄, filtered and concentrated to get crude (7S, 10S)-13-benzyl-10-isobutyl-1, 9, 12-trioxo-2, 8, 11-triazaspiro [4.14] nonadec-17-ene-7-carbaldehyde (B69). It was used for the next step without any purification.

### Diethyl (((7S,10S)-13-benzyl-10-isobutyl-1,9,12-trioxo-2,8,11-triazaspiro[4.14]nonadec-17-en-7-yl)(hydroxy)methyl)phosphonate B23

To a stirred solution of (7S,10S)-13-benzyl-10-isobutyl-1,9,12-trioxo-2,8,11-triazaspiro[4.14]nonadec-17-ene-7-carbaldehyde (B69) (100 mg, 0.207 mmol) in DCM (5 mL) was added DIPEA (0.12 mL, 0.623 mmol) and diethyl phosphite (86 mg, 0.623 mmol) and stirred at RT for 16 h. The progress of the reaction was monitored by TLC and LCMS. After 16 h, the reaction mixture was diluted by DCM (20 mL) and washed with water (2×30 mL) and brine solution (20 mL). Organic layer was dried over anhydrous Na₂SO₄, and evaporated to afford crude residue. It was purified by prep HPLC and afforded diethyl (((7S,10S)-13-benzyl-10-isobutyl-1,9,12-trioxo-2,8,11-triazaspiro[4.14]nonadec-17-en-7-yl)(hydroxy)methyl)phosphonate as formic acid salt B23

TLC system: 10% Methanol in DCM; Rf: 0.1

LCMS (ESI): m/z 620.48[M+H]⁺

### Synthesis of B70 and B71

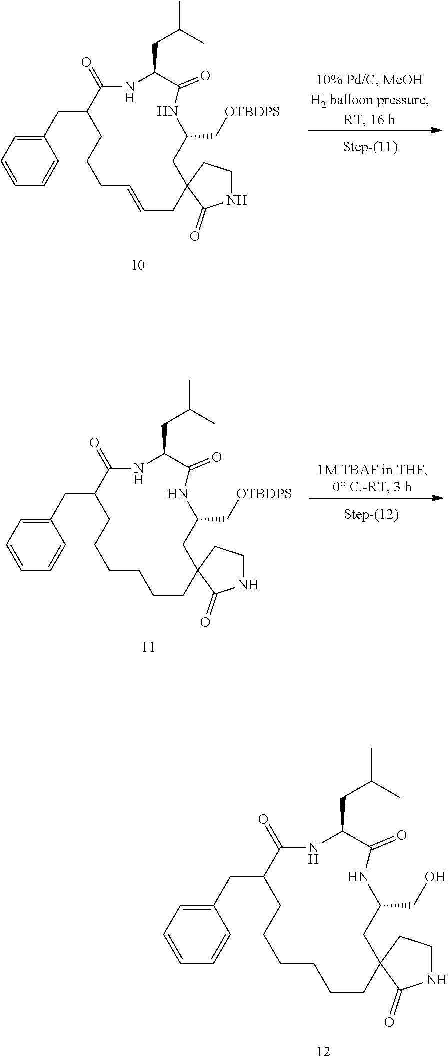

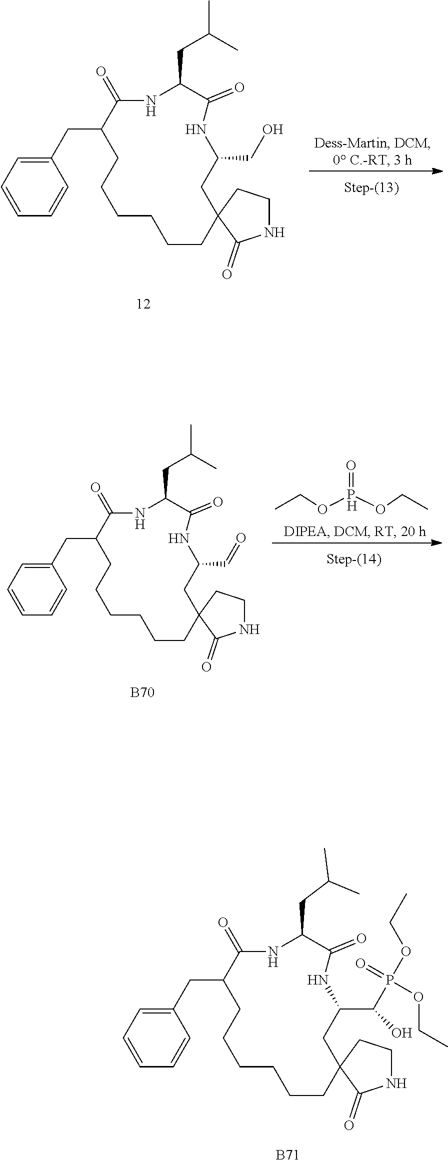

### (7S,10S)-13-Benzyl-7-(((tert-butyldiphenylsilyl)oxy)methyl)-10-isobutyl-2,8,11-triazaspiro[4.14]nonadecane-1,9,12-trione (11)

To a stirred solution of (7S,10S)-13-benzyl-7-(((tert-butyldiphenylsilyl)oxy)methyl)-10-isobutyl-2,8,11-triazaspiro[4.14]nonadec-17-ene-1,9,12-trione (10) (500 mg, 0.6934 mmol) in MeOH (5 mL) added 10% Pd/C (200 mg) and stirred at H₂ atmosphere for 16 h. The progress of the reaction was monitored by TLC and LCMS. Then the reaction mixture was filtered through celite bed and washed with MeOH (2×30 mL), the filtrate was evaporated under reduced pressure to afford crude (7S,10S)-13-benzyl-7-(((tert-butyldiphenylsilyl)oxy)methyl)-10-isobutyl-2,8,11-triazaspiro[4.14]nonadecane-1,9,12-trione (11).

### (7S,10S)-13-benzyl-7-(hydroxymethyl)-10-isobutyl-2,8,11-triazaspiro[4.14]nonadecane-1,9,12-trione (12)

To a stirred solution of (7S,10S)-13-benzyl-7-(((tert-butyldiphenylsilyl)oxy)methyl)-10-isobutyl-2,8,11-triazaspiro[4.14]nonadecane-1,9,12-trione (11) (300 mg, 0.5547 mmol) in THE (10 mL) was added 1M TBAF in THE (0.8 mL, 0.8321 mmol) at RT and stirred at RT for 3 h. The progress of the reaction was monitored by TLC and LCMS. After 3 h, the reaction mixture completely distilled under reduced pressure, crude compound diluted with water (30 mL) and extracted with ethyl acetate (2×50 mL), the combined organic layer was dried over sodium sulfate, concentrated under reduced pressure to afford crude product; this crude was purified by normal phase chromatography with eluting 8% methanol in dichloromethane to afford (7S,10S)-13-benzyl-7-(hydroxymethyl)-10-isobutyl-2,8,11-triazaspiro[4.14]nonadecane-1,9,12-trione (12).

### (7S,10S)-13-benzyl-10-isobutyl-1,9,12-trioxo-2,8,11-triazaspiro[4.14]nonadecane-7-carbaldehyde B70

To a stirred solution of (7S,10S)-13-benzyl-7-(hydroxymethyl)-10-isobutyl-2,8,11-triazaspiro[4.14]nonadecane-1,9,12-trione (12) (50 mg, 0.103 mmol) in DCM (5 mL) at 0° C. was added Dess-Martin periodinane (87 mg, 0.559 mmol) and stirred at RT for 3 h. The progress of the reaction was monitored by TLC and LCMS. After 3 h, the reaction mixture was diluted by DCM (30 mL) and washed with sat. Hypo solution (3×20 mL), sat. NaHCO₃ solution (3×20 mL). Organic layer was dried over anhydrous Na₂SO₄, filtered and concentrated to get crude (7S,10S)-13-benzyl-10-isobutyl-1,9,12-trioxo-2,8,11-triazaspiro[4.14]nonadecane-7-carbaldehyde B70. It was used into next step without any purification.

### Diethyl ((1S)-((7S,10S)-13-benzyl-10-isobutyl-1,9,12-trioxo-2,8,11-triazaspiro[4.14]nonadecan-7-yl)(hydroxy)methyl)phosphonate B71

To a stirred solution of (7S,10S)-13-benzyl-10-isobutyl-1,9,12-trioxo-2,8,11-triazaspiro[4.14]nonadecane-7-carbaldehyde B70 (50 mg, 0.1039 mmol) in DCM (3 mL) was added DIPEA (0.12 mL, 0.207 mmol) and diethyl phosphite (0.1 mL, 0.3105 mmol) and the reaction mixture was stirred at RT for 16 h. The progress of the reaction was monitored by TLC and LCMS. After 16 h, reaction mixture was diluted by DCM (30 mL) and washed with water (2×10 mL) and brine solution. Organic layer was dried over anhydrous Na₂SO₄ and evaporated to afford crude residue. It was purified by prep HPLC and afforded diethyl ((1S)-((7S,10S)-13-benzyl-10-isobutyl-1,9,12-trioxo-2,8,11-triazaspiro[4.14]nonadecan-7-yl)(hydroxy)methyl)phosphonate B71

TLC system: 10% Methanol in DCM; Rf: 0.1

LCMS (ESI): m/z 622.57[M+H]⁺

### Synthesis of Compounds B24 and B21

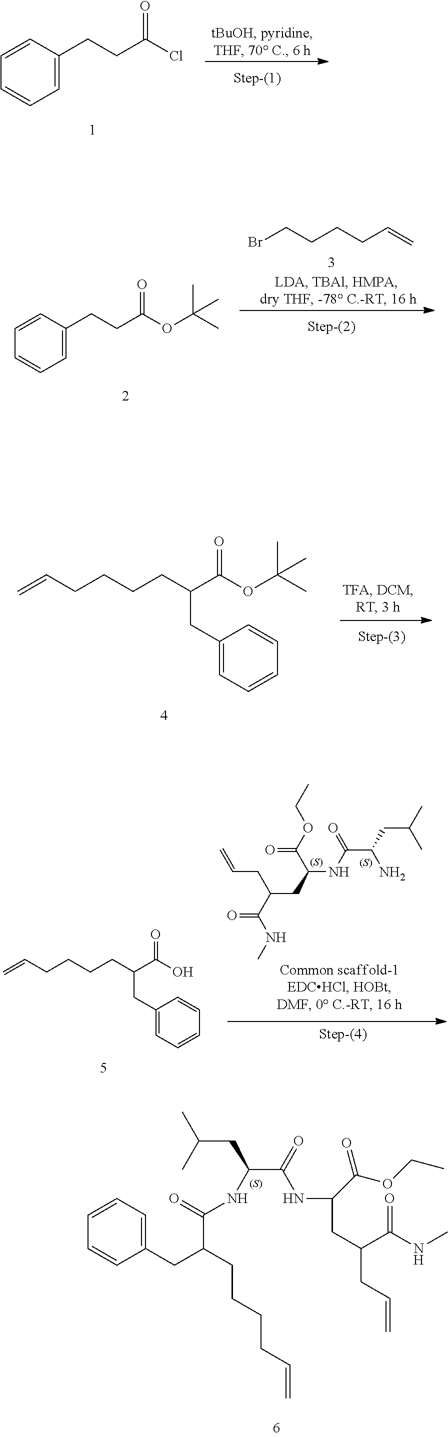

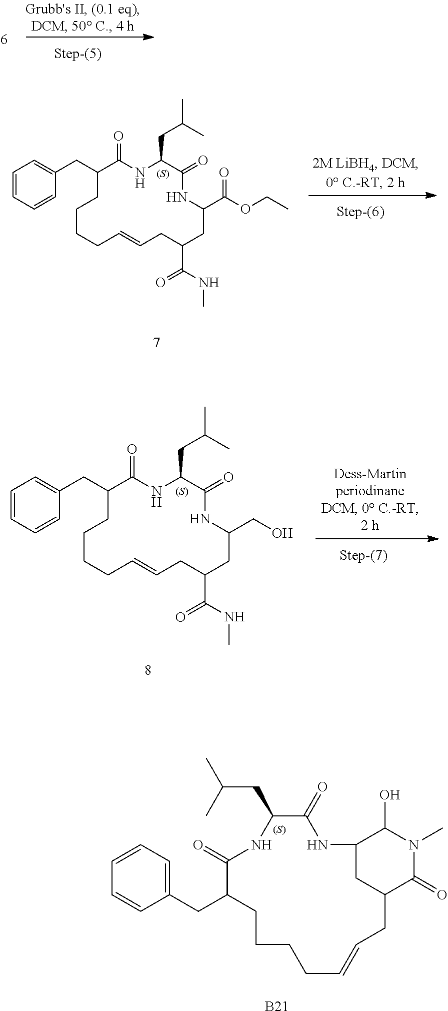

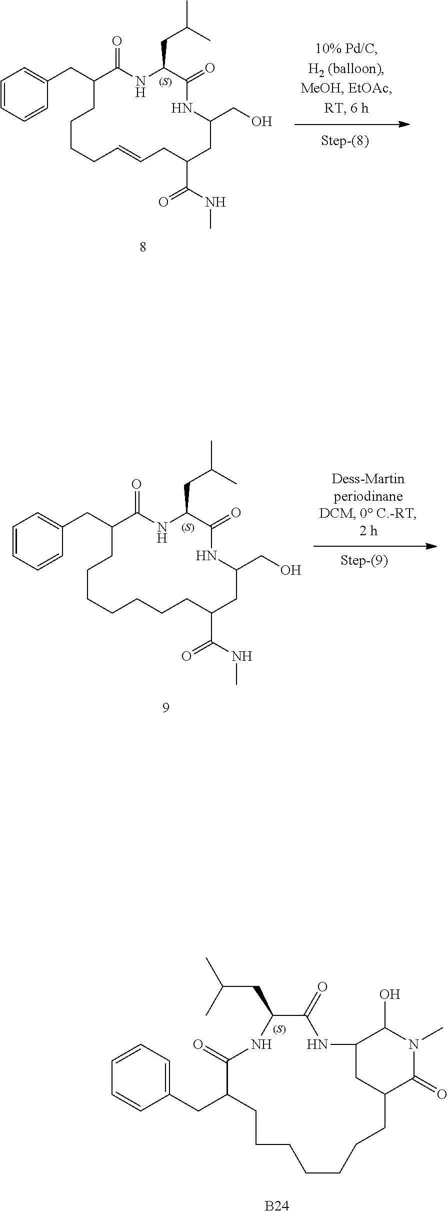

### tert-butyl 3-phenylpropanoate (2)

To a stirred solution of 3-phenylpropanoyl chloride (1) (10 g, 59.5238 mmol) in THE (100 mL) was cooled to 0° C. and added pyridine (9.4 mL, 119.047 mmol) followed by ^{t}BuOH (4.4 mL, 119.0 mmol) and reaction mixture was heated to 70° C. for 6 h. After consumption of starting material, the reaction mixture quenched with water (30 mL) and extracted with EtOAc (2×100 mL). Organic layer was washed with brine solution, dried over Na₂SO₄ and concentrated to get crude. Crude compound was purified by column chromatography to afford pure compound tert-butyl 3-phenylpropanoate (2).

### tert-butyl 2-benzyloct-7-enoate (4)

To a stirred solution of tert-butyl 3-phenylpropanoate (2 g, 9.70 mmol) in THE (20 mL), was added 2M LDA (7.7 mL, 15.53 mmol). Reaction mixture was cooled to −78° C. and stirred for 1 h then added TBAI (179 mg, 4.85 mmol), HMPA (2 mL) and 6-bromohex-1-ene (3) (1.9 g, 11.65 mmol) was added at same temperature. The reaction mixture was stirred at RT for 16 h. After consumption of starting material reaction mixture was quenched with sat. NH₄Cl solution (100 mL) and extracted with EtOAc (2×30 mL). Organic layer was washed with brine (50 mL) then dried over anhydrous Na₂SO₄ and concentrated under reduced pressure to afford crude product; this crude was purified by column chromatography by using silica-gel (100-200) with eluting 2% EtOAc in hexane to afford pure compound tert-butyl 2-benzyloct-7-enoate (4).

### 2-Benzyloct-7-enoic acid (5)

To a stirred solution of tert-butyl 2-benzyloct-7-enoate (4) (2 g, 8.33 mmol) in DCM (20 mL) was added TFA (10 mL) at 0° C. to RT and the mixture was stirred for 3 h. After consumption of starting material. The solvent was concentrated under reduced pressure to afford crude compound (5) 2-benzyloct-7-enoic acid (5). This product was used for next step without any purification.

### Ethyl 2-((2S)-2-(2-benzyloct-7-enamido)-4-methylpentanamido)-4-(methylcarbamoyl) hept-6-enoate (6)

To a stirred solution of 2-benzyloct-7-enoic acid (5) (1.4 g, 6.034 mmol) in DMF (14 mL) was added EDC.HCl (1.7 g, 9.051 mmol), HOBt (1.2 g, 9.051 mmol), DIPEA (3 mL, 18.10 mmol) at 0° C. and reaction mixture was stirred for 15 min at 0° C. then added ethyl (2S)-2-((S)-2-amino-4-methylpentanamido)-4-(methylcarbamoyl)hept-6-enoate (2.4 g, 7.24 mmol) at 0° C. and stirred for 16 h at rt. After consumption of starting material, the reaction mixture was quenched with ice water (100 Ml) and extracted with EtOAc (2×50 mL). Organic layer was washed with brine (100 mL), dried over anhydrous Na₂SO₄ and concentrated under reduced pressure to afford crude product; this crude product was purified by column chromatography by using silica-gel (100-200) with eluting 40% EtOAc in hexane to afford pure compound ethyl 2-((2S)-2-(2-benzyloct-7-enamido)-4-methylpentanamido)-4-(methylcarbamoyl)hept-6-enoate (6).

### Ethyl (2S,E)-15-benzyl-2-isobutyl-7-(methylcarbamoyl)-3,16-dioxo-1,4-diazacyclohexadec-9-ene-5-carboxylate (7)

To a stirred solution of ethyl (2S)-4-(dimethylcarbamoyl)-2-((2S)-2-(2-(hex-5-en-1-yl)nonanamido)-4-methylpentanamido)hept-6-enoate (6) (1 g, 1.733 mmol) in DCM (100 mL) was added Grubb's II (0.147 g, 0.173 mmol) at rt and stirred at 50° C. for 4 h. After consumption of starting material reaction mixture was evaporated under reduced pressure to afford crude product; this crude was purified by normal phase chromatography with eluting 4% methanol in dichloromethane to afforded pure ethyl (2S,5S,E)-7-(dimethylcarbamoyl)-15-heptyl-2-isobutyl-3,16-dioxo-1,4-diazacyclohexadec-9-ene-5-carboxylate (7).

### (2S,E)-15-benzyl-5-(hydroxymethyl)-2-isobutyl-N-methyl-3,16-dioxo-1,4-diazacyclohexadec-9-ene-7-carboxamide (8)

To a stirred solution of ethyl (2S,E)-15-benzyl-2-isobutyl-7-(methylcarbamoyl)-3,16-dioxo-1,4-diazacyclohexadec-9-ene-5-carboxylate (7) (1.5 g, 2.84 mmol) in DCM (15 mL) was added 2M LiBH₄ in THE (4.2 mL, 8.538 mmol) at 0° C., reaction mixture stirred at rt for 2 h. Reaction mixture was quenched with water (70 mL) and extracted with DCM (2×30 mL). Organic layer was washed with brine solution, dried over Na₂SO₄ and concentrated to get crude. Crude compound was purified by normal phase chromatography to afford pure (2S,E)-15-benzyl-5-(hydroxymethyl)-2-isobutyl-N-methyl-3,16-dioxo-1,4-diazacyclohexadec-9-ene-7-carboxamide (8).

### (4S, E)-7-benzyl-18-hydroxy-4-isobutyl-17-methyl-2, 5, 17-triazabicyclo [13.3.1] nonadec-12-ene-3, 6, 16-trione B21

To a stirred solution of (2S,E)-15-benzyl-5-(hydroxymethyl)-2-isobutyl-N-methyl-3,16-dioxo-1,4-diazacyclohexadec-9-ene-7-carboxamide (8) (200 mg, 0.412 mmol) was dissolved in DCM (5 mL) was added Dess-Martin periodinane (349 mg, 0.824 mmol) at 0° C. and stirred at RT for 2 h. Reaction mixture was diluted with DCM and washed with sat hypo solution (3×20 mL) followed by sat NaHCO₃ solution (3×20 mL). Organic layer was dried over Na₂SO₄ and concentrated to get crude. Crude compound was purified by prep HPLC to afford pure (4S,E)-7-benzyl-18-hydroxy-4-isobutyl-17-methyl-2,5,17-triazabicyclo[13.3.1]nonadec-12-ene-3,6,16-trione B21.

TLC system: 10% Methanol in DCM; Rf: 0.4

LCMS (ESI): m/z 484.3 (M+H)⁺

### (2S)-15-benzyl-5-(hydroxymethyl)-2-isobutyl-N-methyl-3,16-dioxo-1,4-diazacyclohexadecane-7-carboxamide (9)

To a stirred solution of (2S,E)-15-benzyl-5-(hydroxymethyl)-2-isobutyl-N-methyl-3,16-dioxo-1,4-diazacyclohexadec-9-ene-7-carboxamide 8 (250 mg, 0515 mmol) in MeOH (2.5 mL) added 10% Pd/C (150 mg) and stirred at H₂ atmosphere for 16 h. After consumption of starting material, reaction mixture was filtered through the celite bed washed with MeOH (100 mL), the filtrate mL's was evaporated under reduced pressure to afford crude (2S)-15-benzyl-5-(hydroxymethyl)-2-isobutyl-N-methyl-3,16-dioxo-1,4-diazacyclohexadecane-7-carboxamide (9).

### (4S)-7-benzyl-18-hydroxy-4-isobutyl-17-methyl-2,5,17-triazabicyclo[13.3.1]nonadecane-3,6,16-trione B24

To a stirred solution of (2S)-15-benzyl-5-(hydroxymethyl)-2-isobutyl-N-methyl-3,16-dioxo-1,4-diazacyclohexadecane-7-carboxamide (9) (200 mg, 0.410 mmol) was dissolved in DCM (2 mL) was added dessmartinperiodinane (348 mg, 0.281 mmol) at 0° C. and stirred at RT for 3 h. Reaction mixture was diluted with DCM and washed with sat hypo solution (3×20 mL) followed by sat NaHCO₃ solution (3×20 mL). Organic layer was dried over Na₂SO₄ and concentrated to get crude. Crude compound was purified by prep HPLC to afford pure (4S)-7-benzyl-18-hydroxy-4-isobutyl-17-methyl-2,5,17-triazabicyclo[13.3.1]nonadecane-3,6,16-trione B24.

TLC system: 10% Methanol in DCM; Rf: 0.4

LCMS (ES 1): m/z 486.44 (M+H)⁺

### Synthesis of Compounds B29, B25, and B26

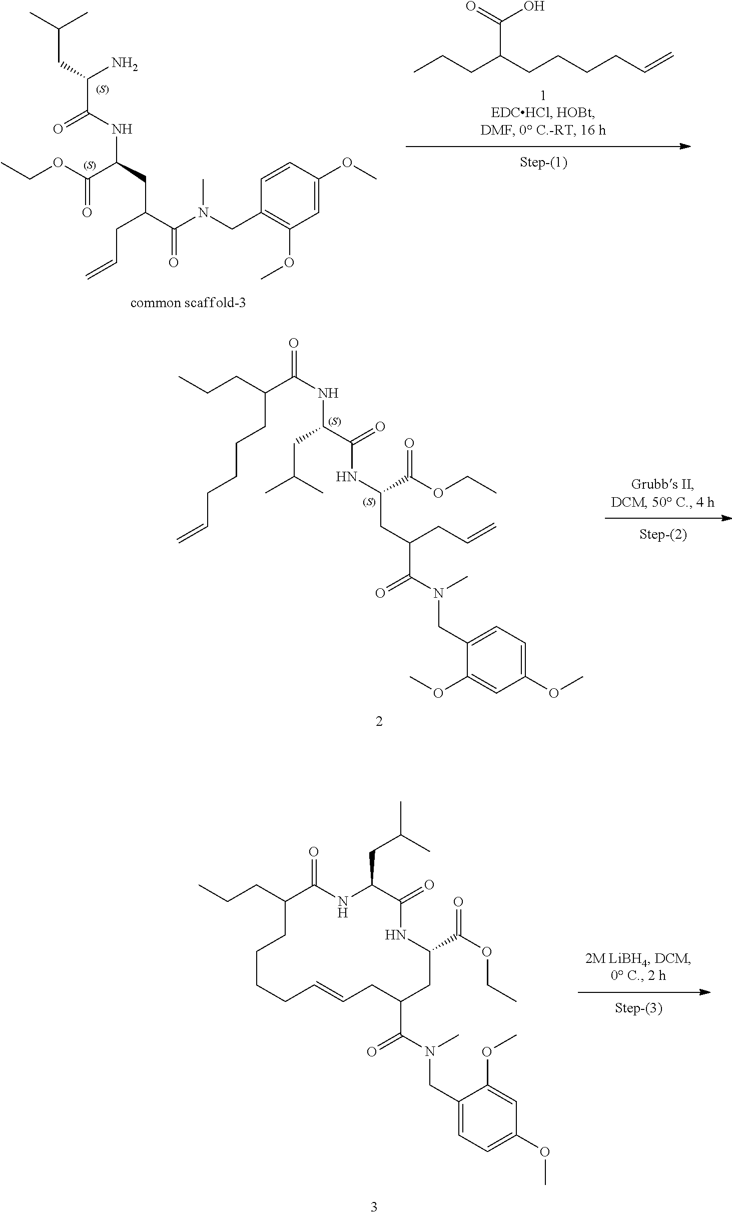

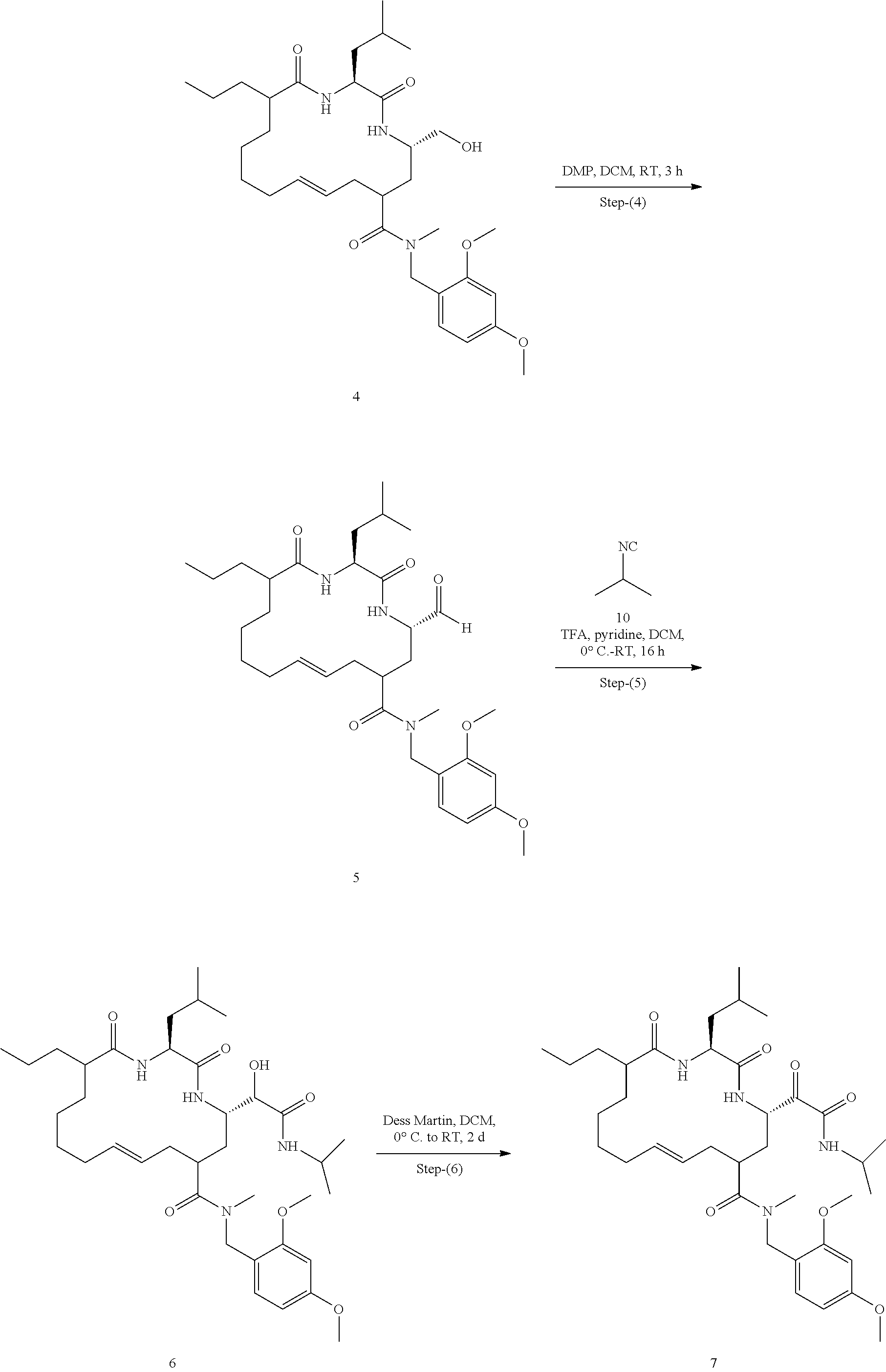

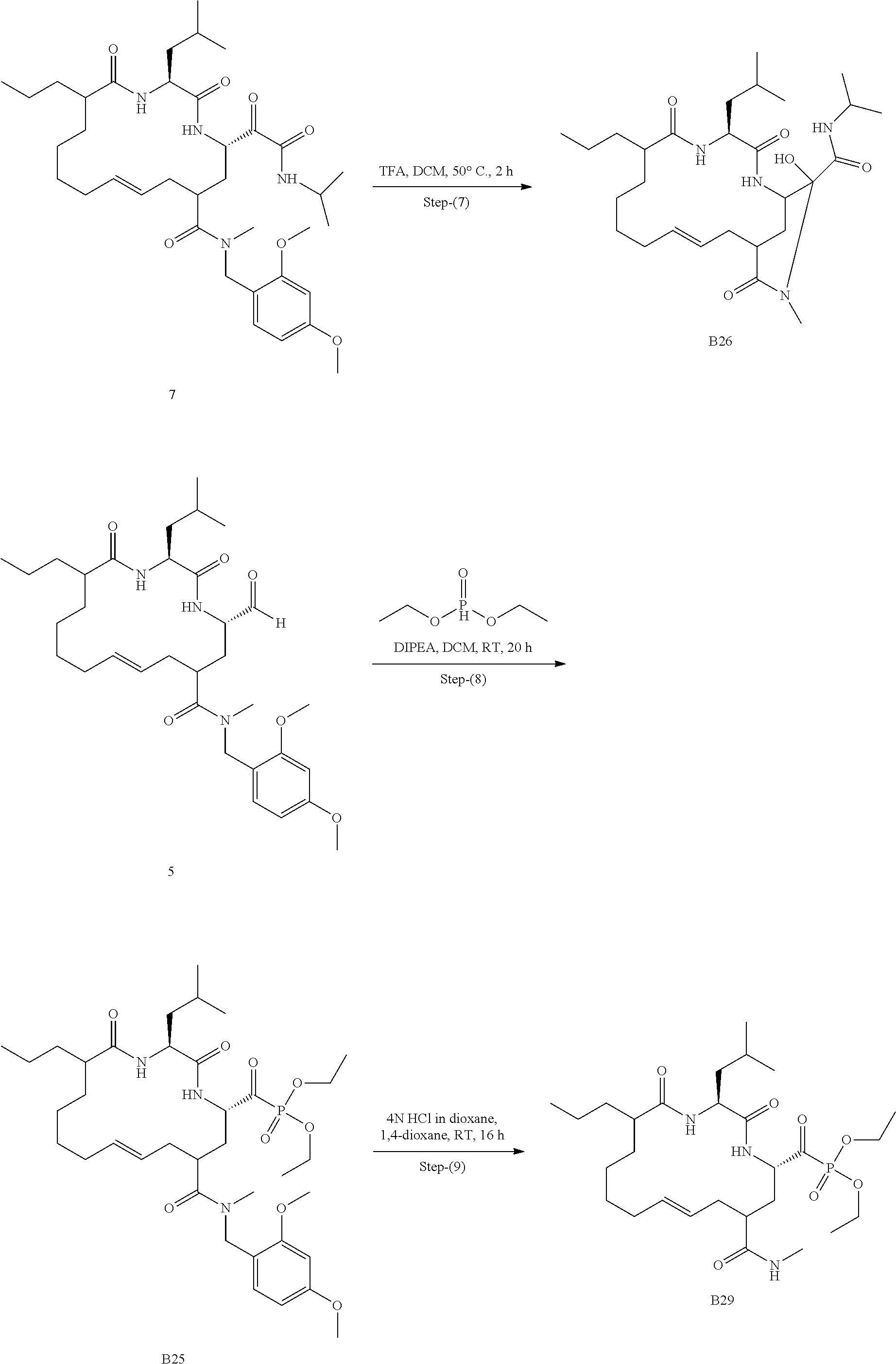

### Ethyl (2S)-4-((2, 4-dimethoxybenzyl)(methyl)carbamoyl)-2-((2S)-4-methyl-2-(2-propyloct-7-enamido)pentanamido)hept-6-enoate (2)

To a stirred solution of 2-propyloct-7-enoic acid (1) (400 mg, 2.17 mmol) in DMF (10 mL) was added EDC.HCl (622 mg, 3.26 mmol), HOBt (440 mg, 3.26 mmol), DIPEA (1.2 mL, 6.52 mmol) at 0° C. and reaction mixture was stirred for 15 min at same temperature then added ethyl (2S)-2-((S)-2-amino-4-methylpentanamido)-4-((2,4-dimethoxybenzyl)(methyl)carbamoyl)hept-6-enoate (1.37 g, 2.61 mmol) (Synthesis reported in common scaffold-3) at 0° C. and stirred at RT for 16 h. After consumption of starting material, the reaction mixture was quenched with ice water (50 mL) and extracted with EtOAc (2×50 mL). Combined organic layer was washed with brine (100 mL), dried over anhydrous Na₂SO₄ and concentrated under reduced pressure to afford crude product; this crude product was purified by silica gel (100-200 mesh) column chromatography eluting with 60% EtAOc in hexane to afford ethyl (2S)-4-((2,4-dimethoxybenzyl)(methyl)carbamoyl)-2-((2S)-4-methyl-2-(2-propyloct-7-enamido)pentanamido)hept-6-enoate (2).

### Ethyl (2S, 5S, E)-7-((2, 4-dimethoxybenzyl)(methyl)carbamoyl)-2-isobutyl-3,16-dioxo-15-propyl-1,4-diazacyclohexadec-9-ene-5-carboxylate (3)

To a stirred solution of ethyl (2S)-4-((2,4-dimethoxybenzyl)(methyl)carbamoyl)-2-((2S)-4-methyl-2-(2-propyloct-7-enamido)pentanamido)hept-6-enoate (2) (400 mg, 0.61 mmol) in DCM (10 mL) was added Grubb's II (51 mg, 0.61 mmol) at rt and stirred at 50° C. for 4 h. After consumption of starting material, reaction mixture was evaporated under reduced pressure to afford crude product; this crude was purified by reverse phase chromatography with eluting 40% ACN in 0.1% formic acid in water, to afforded ethyl (2S,5S,E)-7-((2,4-dimethoxybenzyl)(methyl)carbamoyl)-2-isobutyl-3,16-dioxo-15-propyl-1,4-diazacyclohexadec-9-ene-5-carboxylate (3).

### (2S, 5S, E)-N-(2, 4-dimethoxybenzyl)-5-(hydroxymethyl)-2-isobutyl-N-methyl-3, 16-dioxo-15-propyl-1, 4-diazacyclohexadec-9-ene-7-carboxamide

To a stirred solution of ethyl (2S,5S,E)-7-((2,4-dimethoxybenzyl)(methyl)carbamoyl)-2-isobutyl-3,16-dioxo-15-propyl-1,4-diazacyclohexadec-9-ene-5-carboxylate (3) (300 mg, 0.47 mmol) in DCM (3 mL) at 0° C. added lithium borohydride (2M in THF) (0.7 mL, 0.14 mmol) and stirred at room temperature for 2 h. Reaction mixture quenched with ice water (50 mL) and extracted with EtOAc (2×30 mL), dried over anhydrous Na₂SO₄ and evaporated under reduced pressure. The crude residue was purified by Grace normal phase using 5% MeOH/DCM to afford (2S, 5S, E)-N-(2,4-dimethoxybenzyl)-5-(hydroxymethyl)-2-isobutyl-N-methyl-3,16-dioxo-15-propyl-1,4-diazacyclohexadec-9-ene-7-carboxamide (4).

### (2S, 5S, E)-N-(2, 4-dimethoxybenzyl)-5-formyl-2-isobutyl-N-methyl-3, 16-dioxo-15-propyl-1, 4-diazacyclohexadec-9-ene-7-carboxamide (5)

To a stirred solution of (2S,5S,E)-N-(2,4-dimethoxybenzyl)-5-(hydroxymethyl)-2-isobutyl-N-methyl-3,16-dioxo-15-propyl-1,4-diazacyclohexadec-9-ene-7-carboxamide (4) (400 mg, 0.68 mmol) in DCM (4 mL) at 0° C. was added Dess-Martin periodinane (577 mg, 1.36 mmol and stirred at room temperature for 16 h. Reaction mixture was diluted with ice water (50 mL), extracted with DCM (2×30 mL), organic layer washed with sat. NaHCO₃ (2×30 mL) and hypo solution (2×30 mL) dried over anhydrous Na₂SO₄ and evaporated under reduced pressure. The crude residue was afforded (2S,5S,E)-N-(2,4-dimethoxybenzyl)-5-formyl-2-isobutyl-N-methyl-3,16-dioxo-15-propyl-1,4-diazacyclohexadec-9-ene-7-carboxamide (5). The residue was directly used by next step.

### (2S, 5S, E)-N-(2, 4-dimethoxybenzyl)-5-((R)-1-hydroxy-2-(isopropylamino)-2-oxoethyl)-2-isobutyl-N-methyl-3, 16-dioxo-15-propyl-1, 4-diazacyclohexadec-9-ene-7-carboxamide (6)

To a stirred solution of (2S,5S,E)-N-(2,4-dimethoxybenzyl)-5-formyl-2-isobutyl-N-methyl-3,16-dioxo-15-propyl-1,4-diazacyclohexadec-9-ene-7-carboxamide (5) (0.200 g, 0.34 mmol) in DCM (2 mL) at 0° C. was added pyridine (0.1 mL, 1.367 mmol), 2-isocyanopropane (10) (0.05 mL, 0.68 mmol) and TFA (0.05 mL, 0.683 mmol) and stirred at rt for 16 h. Reaction mixture was concentrated under reduced pressure to get the crude residue was quenched with ice water (2×50 mL) and extracted with DCM (2×20 mL), dried over anhydrous Na₂SO₄, concentrated under reduced pressure to get the crude residue was purified by (100-200) column chromatography to afford compound (2S,5S,E)-N-(2,4-dimethoxybenzyl)-5-((R)-1-hydroxy-2-(isopropylamino)-2-oxoethyl)-2-isobutyl-N-methyl-3,16-dioxo-15-propyl-1,4-diazacyclohexadec-9-ene-7-carboxamide (6).

### (2S, 5S, E)-N-(2, 4-dimethoxybenzyl)-2-isobutyl-5-(2-(isopropylamino)-2-oxoacetyl)-N-methyl-3, 16-dioxo-15-propyl-1, 4-diazacyclohexadec-9-ene-7-carboxamide (7)

To a stirred solution of (2S,5S,E)-N-(2,4-dimethoxybenzyl)-5-((R)-1-hydroxy-2-(isopropylamino)-2-oxoethyl)-2-isobutyl-N-methyl-3,16-dioxo-15-propyl-1,4-diazacyclohexadec-9-ene-7-carboxamide (7) (100 mg, 0.148 mmol) in DCM (10 mL) at 0° C. was added Dess-Martin periodinane (126 mg, 0.297 mmol) and stirred at room temperature for 16 h. Reaction mixture was diluted with ice water (50 mL), extracted with DCM (2×30 mL), organic layer washed with sat.NaHCO₃ and hypo solution (2×40 mL) and dried over anhydrous Na₂SO₄ and evaporated under reduced pressure. The crude residue was purified by silica gel (100-200 mesh) column purification to afforded (2S, 5S, E)-N-(2, 4-dimethoxybenzyl)-2-isobutyl-5-(2-(isopropylamino)-2-oxoacetyl)-N-methyl-3, 16-dioxo-15-propyl-1, 4-diazacyclohexadec-9-ene-7-carboxamide 7.

### (4S, E)-18-hydroxy-4-isobutyl-N-isopropyl-17-methyl-3,6,16-trioxo-7-propyl-2,5,17-triazabicyclo[13.3.1]nonadec-12-ene-18-carboxamide B26

To a stirred solution of (2S,5S,E)-N-(2,4-dimethoxybenzyl)-2-isobutyl-5-(2-(isopropylamino)-2-oxoacetyl)-N-methyl-3,16-dioxo-15-propyl-1,4-diazacyclohexadec-9-ene-7-carboxamide (7) (200 mg, 0.29 mmol) in DCM (2 mL) was added TFA (1 mL) and slowly heated to 50° C. for 3 h. Solvent was evaporated under reduced pressure to afford crude compound. Crude residue was basified with aq. NaHCO₃ (10 mL) and extracted with EtOAc (2×30 mL), dried over anhydrous Na₂SO₄ and evaporated under reduced pressure to get the crude residue submitted by prep-HPLC to afford (4S,E)-18-hydroxy-4-isobutyl-N-isopropyl-17-methyl-3,6,16-trioxo-7-propyl-2,5,17-triazabicyclo[13.3.1]nonadec-12-ene-18-carboxamide B26.

TLC system: 10% Methanol in dichloromethane; Rf: 0.5

LCMS (ESI): m/z 521.3 (M+H)⁺

### Diethyl (((2S,5S,E)-7-((2,4-dimethoxybenzyl)(methyl)carbamoyl)-2-isobutyl-3,16-dioxo-15-propyl-1,4-diazacyclohexadec-9-en-5-yl)(hydroxy)methyl)phosphonate B25

To a stirred solution of (2S,5S,E)-N-(2,4-dimethoxybenzyl)-5-formyl-2-isobutyl-N-methyl-3,16-dioxo-15-propyl-1,4-diazacyclohexadec-9-ene-7-carboxamide (200 mg, 0.34 mmol) in DCM (5 mL) added DIPEA (0.2 mL, 1.02 mmol) followed by added diethyl phosphite (0.13 mL, 1.02 mmol) at ° C., then reaction mixture stirred at RT for 16 h and then reaction mixture quenched with ammonium chloride (15 mL) and extracted with DCM (2×20 mL). Combined organic layer dried over anhydrous Na₂SO₄, and evaporated to afford crude residue. It was purified prep HPLC to afforded Diethyl (((2S,5S,E)-7-((2,4-dimethoxybenzyl)(methyl)carbamoyl)-2-isobutyl-3,16-dioxo-15-propyl-1,4-diazacyclohexadec-9-en-5-yl)(hydroxy)methyl)phosphonate B25.

TLC system: 5% Methanol in DCM; Rf: 0.4

LCMS (ESI): m/z 724.3 (M+H)⁺

### Diethyl (hydroxy ((2S, 5S, E)-2-isobutyl-7-(methylcarbamoyl)-3, 16-dioxo-15-propyl-1, 4-diazacyclohexadec-9-en-5-yl) methyl) phosphonate B29

To a stirred solution of diethyl (((2S,5S,E)-7-((2,4-dimethoxybenzyl)(methyl)carbamoyl)-2-isobutyl-3,16-dioxo-15-propyl-1,4-diazacyclohexadec-9-en-5-yl)(hydroxy)methyl)phosphonate B25 (90 mg, 0.12 mmol) in 1,4 dioxane (2 mL) was added 4N dioxane.HCl (4 mL) and stirred for 16 h. Solvent was evaporated under reduced pressure to afford crude compound. Crude residue was basified with aq. NaHCO₃ (10 mL) and extracted with EtOAc (2×20 mL), dried over anhydrous Na₂SO₄ and evaporated under reduced pressure to get the crude residue submitted by prep-HPLC to afford diethyl (hydroxy((2S,5S,E)-2-isobutyl-7-(methylcarbamoyl)-3,16-dioxo-15-propyl-1,4-diazacyclohexadec-9-en-5-yl)methyl)phosphonate B29.

TLC system: 10% Methanol in dichloromethane; Rf: 0.3

LCMS (ESI): m/z 521.3 (M+H)⁺

### Synthesis of Compounds B27 and B28

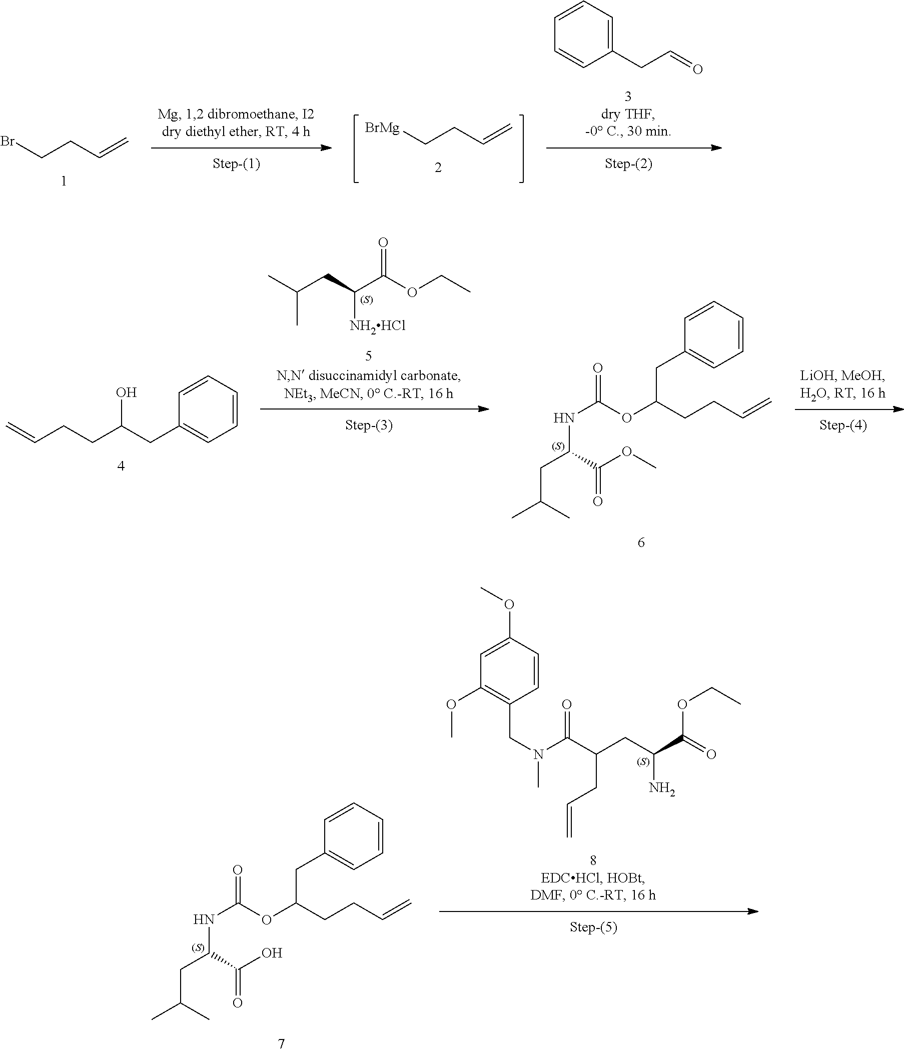

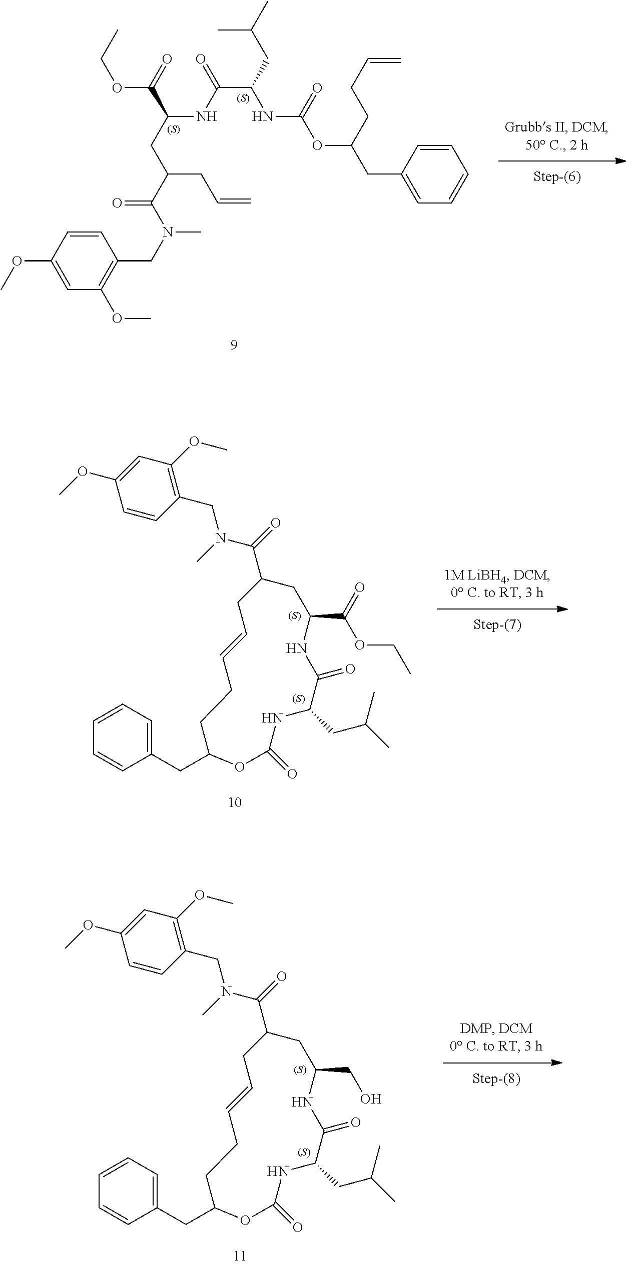

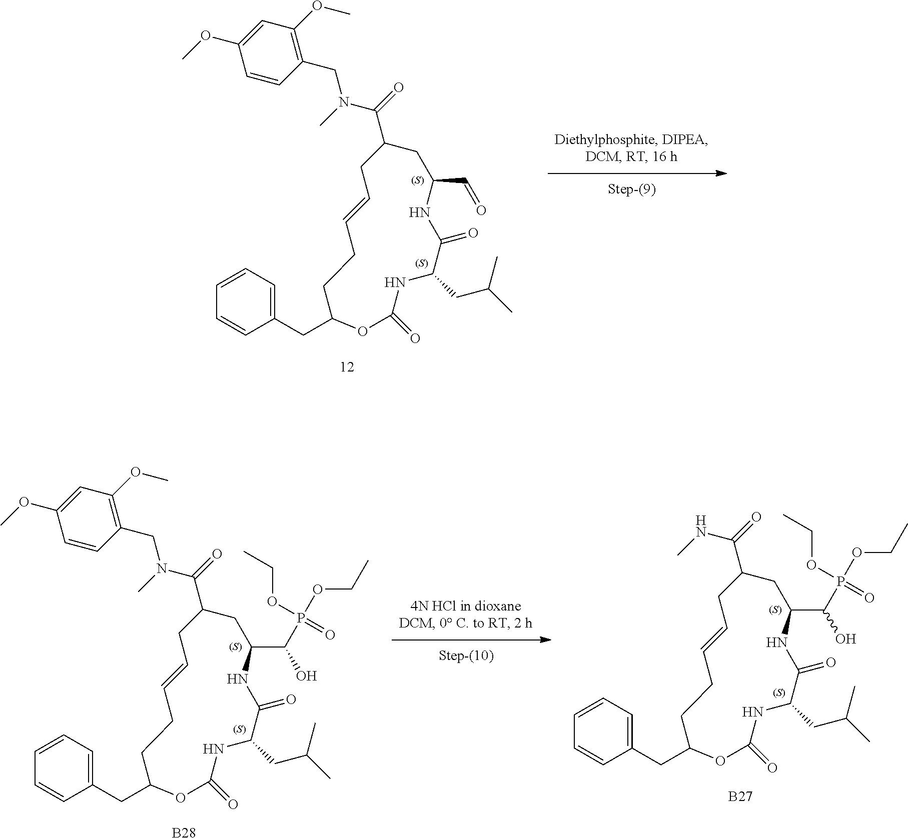

### 1-phenylhex-5-en-2-ol (4)

To a dried magnesium turnings (2.57 g, 107.46 mmol) and catalytic iodine (150 mg, 4.477 mmol) in a three neck RBF added dry diethyl ether (170 mL), 1,2-dibromo ethane (170 mg, 0.89 mmol) and 4-bromobut-1-ene (1) (12 g, 89.55 mmol) at RT (observed exothermic and refluxed the reaction) reaction was stirred at room temperature for 4 h. The resultant Grignard mixture (2) was used as such in the next step.

To a stirred solution of 2-phenylacetaldehyde (3) (5 g, 41.66 mmol) in dry diethyl ether (50 mL) at −78° C. was slowly added to above freshly prepared but-3-en-1-ylmagnesium bromide (2) (13 g, 83.33 mmol) drop wise. The reaction mixture was stirred at same temperature for 30 min. The reaction mixture was quenched with sat. NH₄Cl (100 mL), extracted with EtOAc (2×200 mL), dried over anhydrous Na₂SO₄ and evaporated under vacuum. Obtained crude was purified by silica gel (100-200 mesh) column eluting with 2% EtOAc and pet ether to afford 1-phenylhex-5-en-2-ol (4).

### Methyl(((1-phenylhex-5-en-2-yl)oxy)carbonyl)-L-leucinate (6)

To a stirred solution of 1-phenylhex-5-en-2-ol (4) (2.5 g, 14.204 mmol) in acetonitrile (50 mL) added NEt₃ (5.9 mL) at rt and N,N-disuccinamidyl carbonate (9 g, 35.511 mmol) and stirred at room temperature for 16 h. Reaction mixture was evaporated under reduced pressure. Crude was diluted with ice water (100 mL), extracted with EtOAc (2×50 mL), dried over anhydrous Na₂SO₄ and evaporated under reduced pressure obtained crude. The crude residue was taken in DCM (120 mL) added NEt₃ (6.2 mL, 44.86 mmol)) and methyl L-leucinate hydrochloride (3.09 g, 17.03 mmol) at 0° C. and stirred at room temperature for 16 h. The reaction mixture was quenched with ice water (100 mL) and extracted with DCM (2×100 mL). Combined organic layer was washed with brine (60 mL), dried over anhydrous Na₂SO₄ and concentrated under reduced pressure. Crude was purified through 100-200 silica gel column chromatography by eluting with 2% EtOAc in pet ether to afford Methyl(((1-phenylhex-5-en-2-yl)oxy)carbonyl)-L-leucinate (6).

### (((1-phenylhex-5-en-2-yl)oxy)carbonyl)-L-leucine (7)

To a stirred solution of methyl (((1-phenylhex-5-en-2-yl)oxy)carbonyl)-L-leucinate (6) (3.2 g, 9.22 mmol) in THE (15 mL), MeOH (10 mL) and H₂O (4 mL) was added LiOH (0.88 g, 36.88 mmol) at rt and stirred at room temperature for 16 h. Reaction mixture was distilled off under reduced pressure, crude compound acidified to pH ˜4 with 2N HCl and extracted with EtOAc (2×200 mL), dried over anhydrous Na₂SO₄, concentrated under reduced pressure to afford (((1-phenylhe5-en-2-yl)oxy)carbonyl)-L-leucine (7).

### Ethyl (2S)-4-((2,4-dimethoxybenzyl)(methyl)carbamoyl)-2-((2S)-4-methyl-2-((((1-phenylhex-5-en-2-yl)oxy)carbonyl)amino)pentanamido)hept-6-enoate (9)

To a stirred solution of (((1-phenylhex-5-en-2-yl)oxy)carbonyl)-L-leucine (7) (2 g, 6.006 mmol) in DMF (40 mL) were added EDC.HCl (1.7 g, 9.009 mmol), HOBT (1.2 g, 9.009 mmol) and Et₃N (2.5 mL, 18.01 mmol) at 0° C. and stirred for 10 min, was added Ethyl (2S)-2-amino-4-((2,4-dimethoxybenzyl)(methyl)carbamoyl)hept-6-enoate (8) (2.4 g, 6.60 mmol) (Synthesis was reported in common scarfold-3) at 0° C. and stirred at room temperature for 16 h. Reaction mixture was diluted with ice water (150 mL), extracted with EtOAc (2×150 mL), dried over anhydrous Na₂SO₄ and evaporated under reduced pressure. The crude residue was purified by column Grace normal phase with eluent 60% EtOAc in pet ether to afford ethyl (2S)-4-((2,4-dimethoxybenzyl)(methyl)carbamoyl)-2-((2S)-4-methyl-2-((((1-phenylhex-5-en-2-yl)oxy)carbonyl)amino)pentanamido)hept-6-enoate (9).

### Ethyl (4S,7S,E)-15-benzyl-9-((2,4-dimethoxybenzyl)(methyl)carbamoyl)-4-isobutyl-2,5-dioxo-1-oxa-3,6-diazacyclopentadec-11-ene-7-carboxylate (10)

To a stirred solution of afford Ethyl (2S)-4-((2,4-dimethoxybenzyl)(methyl)carbamoyl)-2-((2S)-4-methyl-2-((((1-phenylhex-5-en-2-yl)oxy)carbonyl)amino)pentanamido)hept-6-enoate (9) (2.3 g, 3.318 mmol) in DCM (45 mL) was added Grubb's II catalyst (420 mg, 0.497 mmol) at room temperature and stirred at 50° C. for 3 h. Reaction mixture was quenched with water (50 mL) and extracted with DCM (2×50 mL). Combined organic layer were washed with brine solution (30 mL), dried over anhydrous Na₂SO₄ and concentrated under reduced pressure to afford crude. Crude was purified by Grace normal Phase with eluent 3% MeOH in DCM to afford Ethyl (4S,7S,E)-15-benzyl-9-((2,4-dimethoxybenzyl)(methyl)carbamoyl)-4-isobutyl-2,5-dioxo-1-oxa-3,6-diazacyclopentadec-11-ene-7-carboxylate (10).

### (4S,7S,E)-15-benzyl-N-(2,4-dimethoxybenzyl)-7-(hydroxymethyl)-4-isobutyl-N-methyl-2,5-dioxo-1-oxa-3,6-diazacyclopentadec-11-ene-9-carboxamide (11)

To a stirred solution of Ethyl (4S,7S,E)-15-benzyl-9-((2,4-dimethoxybenzyl)(methyl)carbamoyl)-4-isobutyl-2,5-dioxo-1-oxa-3,6-diazacyclopentadec-11-ene-7-carboxylate (10) (1.2 g, 1.92 mmol) in DCM (15 mL) was added 2M LiBH₄ in THE (1.9 mL, 3.802 mmol) at 0° C. reaction mixture stirred at rt for 2 h. Reaction mixture was quenched with water (70 mL) and extracted with DCM (2×30 mL). Organic layer was washed with brine solution, dried over Na₂SO₄ and concentrated to get crude. Crude compound was purified by normal phase chromatography to afford pure (4S,7S,E)-15-benzyl-N-(2,4-dimethoxybenzyl)-7-(hydroxymethyl)-4-isobutyl-N-methyl-2,5-dioxo-1-oxa-3,6-diazacyclopentadec-11-ene-9-carboxamide (11).

### (4S,7S,E)-15-benzyl-N-(2,4-dimethoxybenzyl)-7-formyl-4-isobutyl-N-methyl-2,5-dioxo-1-oxa-3,6-diazacyclopentadec-11-ene-9-carboxamide (12)

To a stirred solution of (4S,7S,E)-15-benzyl-N-(2,4-dimethoxybenzyl)-7-(hydroxymethyl)-4-isobutyl-N-methyl-2,5-dioxo-1-oxa-3,6-diazacyclopentadec-11-ene-9-carboxamide (11) (250 mg, 0.401 mmol) was dissolved in DCM (5 mL) was added Dess-Martin periodinane (340 mg, 0.802 mmol) at 0° C. and stirred at RT for 3 h. Reaction mixture was diluted with DCM and washed with sat hypo solution (3×20 mL) followed by sat NaHCO₃ solution (3×20 mL). Organic layer was dried over Na₂SO₄ and concentrated to afford (4S,7S,E)-15-benzyl-N-(2,4-dimethoxybenzyl)-7-formyl-4-isobutyl-N-methyl-2,5-dioxo-1-oxa-3,6-diazacyclopentadec-11-ene-9-carboxamide (12).

### Diethyl ((1S)-((4S,7S,E)-15-benzyl-9-((2,4-dimethoxybenzyl)(methyl)carbamoyl)-4-isobutyl-2,5-dioxo-1-oxa-3,6-diazacyclopentadec-11-en-7-yl)(hydroxy)methyl)phosphonate B28

To a stirred solution of (4S,7S,E)-15-benzyl-N-(2,4-dimethoxybenzyl)-7-formyl-4-isobutyl-N-methyl-2,5-dioxo-1-oxa-3,6-diazacyclopentadec-11-ene-9-carboxamide (12) (230 mg, 0.37 mmol) in DCM (4 mL) was added DIPEA (0.2 mL, 1.11 mmol) and diethyl phosphite (0.14 mL, 0.11 mmol). Then reaction mixture was stirred at RT for 16 h (monitoring by TLC indicated complete disappearance of the starting material) and reaction mixture was taken in to DCM (20 mL) and washed with water (2×30 mL) and brine solution. Organic layer was dried over anhydrous Na₂SO₄, and evaporated to afford crude residue. It was purified by prep HPLC and afforded diethyl ((1S)-((4S,7S,E)-15-benzyl-9-((2,4-dimethoxybenzyl)(methyl)carbamoyl)-4-isobutyl-2,5-dioxo-1-oxa-3,6-diazacyclopentadec-11-en-7-yl)(hydroxy)methyl)phosphonate B28

TLC system: 5% MeOH in DCM; Rf: 0.4

LCMS (ESI): m/z 760.53 (M+H)⁺

### Diethyl (((4S,7S,E)-15-benzyl-4-isobutyl-9-(methylcarbamoyl)-2,5-dioxo-1-oxa-3,6-diazacyclopentadec-11-en-7-yl)(hydroxy)methyl)phosphonate B27

To a stirred solution of diethyl ((1S)-((4S,7S,E)-15-benzyl-9-((2,4-dimethoxybenzyl)(methyl)carbamoyl)-4-isobutyl-2,5-dioxo-1-oxa-3,6-diazacyclopentadec-11-en-7-yl)(hydroxy)methyl)phosphonate B28 (220 mg, 0.28 mmol) in DCM (4 mL) at 0° C. was added 4N HCl in dioxane (1.1 mL) (5 vol.) drop wise and the resulting reaction mixture was stirred for 2 h at RT. After consumption of starting material, the volatiles were removed under vacuum, residue was neutralized with sat. NaHCO₃, extracted with EtOAc (3×50 mL) and evaporated to yield the title compound after purification by HPLC.

TLC system: 10% MeOH in DCM; Rf: 0.35

LCMS (ESI): m/z 610.3 (M+H)^{+I}

### Synthesis of Compounds B31, B38, and B30

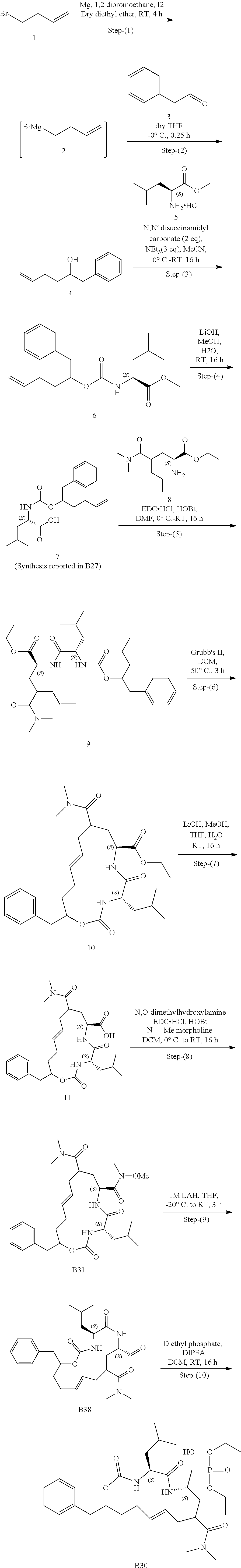

### Ethyl (2S)-4-(dimethylcarbamoyl)-2-((2S)-4-methyl-2-((((1-phenylhex-5-en-2-yl)oxy)carbonyl)amino)pentanamido)hept-6-enoate (9)

To a stirred solution of (((1-phenylhex-5-en-2-yl)oxy)carbonyl)-L-leucine (7) (4 g, 12.012 mmol) in DMF (60 mL), were added EDC.HCl (3.4 g, 18.018 mmol), HOBT (2.4 g, 18.018 mmol) and Et₃N (5 mL, 36.036 mmol) at 0° C. and continued to stirred at 0° C. for 10 min, was added ethyl (2S)-2-amino-4-(dimethylcarbamoyl)hept-6-enoate (8) (3.19 g, 13.213 mmol) (Synthesis was reported in common scarfold-3) at 0° C. and the reaction mixture was stirred at room temperature for 16 h. The progress of the reaction was monitored by TLC and LCMS. After 16 h, the reaction mixture was diluted with ice water (150 mL), extracted with EtOAc (2×150 mL). The organic layer was dried over anhydrous Na₂SO₄ and evaporated under reduced pressure. The crude residue was purified by column Grace normal phase with eluent 60% EtOAc in pet. ether to afford ethyl (2S)-4-(dimethylcarbamoyl)-2-((2S)-4-methyl-2-((((1-phenylhex-5-en-2-yl)oxy)carbonyl)amino)pentanamido) hept-6-enoate (9).

### Ethyl (4S,7S)-15-benzyl-9-(dimethylcarbamoyl)-4-isobutyl-2,5-dioxo-1-oxa-3,6-diazacyclopentadec-11-ene-7-carboxylate (10)

To a stirred solution ethyl (2S)-4-(dimethylcarbamoyl)-2-((2S)-4-methyl-2-((((1-phenylhex-5-en-2-yl)oxy)carbonyl)amino)pentanamido) hept-6-enoate (9) (500 mg, 0.897 mmol) in DCM (10 mL) was added Grubb's II catalyst (113 mg, 0.134 mmol) at RT and then the reaction mixture was stirred at 50° C. for 3 h. The progress of the reaction was monitored by TLC and LCMS. After 3 h, the reaction mixture was quenched with water (20 mL) and extracted with DCM (2×25 mL). Combined organic layer were washed with brine solution (20 mL), dried over anhydrous Na₂SO₄ and concentrated under reduced pressure to afford crude. Crude was purified by Grace normal Phase with eluent 3% MeOH in DCM to afford ethyl (4S,7S)-15-benzyl-9-(dimethylcarbamoyl)-4-isobutyl-2,5-dioxo-1-oxa-3,6-diazacyclopentadec-11-ene-7-carboxylate (10).

### (4S,7S)-15-benzyl-9-(dimethylcarbamoyl)-4-isobutyl-2,5-dioxo-1-oxa-3,6-diazacyclopentadec-11-ene-7-carboxylic acid (11)

To a stirred solution of ethyl (4S,7S)-15-benzyl-9-(dimethylcarbamoyl)-4-isobutyl-2,5-dioxo-1-oxa-3,6-diazacyclopentadec-11-ene-7-carboxylate (10) (2.6 g, 4.914 mmol) in THE (12 mL), MeOH (6 mL) and H₂O (4 mL) was added LiOH (0.47 g, 19.659 mmol) and the reaction mixture was stirred at RT for 16 h. The progress of the reaction was monitored by TLC and LCMS. The reaction mixture was distilled off under reduced pressure, crude compound acidified to pH ˜4 with 2N HCl and extracted with EtOAc (2×100 mL). The combined organic layer was dried over anhydrous Na₂SO₄, concentrated under reduced pressure to afford (4S,7S)-15-benzyl-9-(dimethylcarbamoyl)-4-isobutyl-2,5-dioxo-1-oxa-3,6-diazacyclopentadec-11-ene-7-carboxylic acid 11.

### (4S,7S)-15-Benzyl-4-isobutyl-N7-methoxy-N7,N9,N9-trimethyl-2,5-dioxo-1-oxa-3,6-diazacyclopentadec-11-ene-7,9-dicarboxamide B31

To a stirred solution of (4S,7S)-15-benzyl-9-(dimethylcarbamoyl)-4-isobutyl-2,5-dioxo-1-oxa-3,6-diazacyclopentadec-11-ene-7-carboxylic acid 11 (1 g, 1.9801 mmol) in THE (10 mL) and DCM (10 mL) were added Et₃N (0.83 mL, 5.94 mmol), EDC.HCl (0.56 g, 2.97 mmol) and HOBT (0.4 g, 2.97 mmol) at 0° C. and the reaction mixture was stirred at 0° C. for another 10 min, Then N,O-dimethyl hydroxylamine (0.38 g, 3.96 mmol) was added at 0° C. and the reaction mixture was brought to RT and stirred for another 16 h. The progress of the reaction was monitored by TLC and LCMS. After 16 h, reaction mixture was diluted with ice water (50 mL), extracted with EtOAc (2×100 mL), dried over anhydrous Na₂SO₄ and evaporated under reduced pressure. The crude residue was purified by Grace reverse phase by eluting with 40% 0.1% Formic acid in water and acetonitrile to afford (4S,7S)-15-benzyl-4-isobutyl-N7-methoxy-N7,N9,N9-trimethyl-2,5-dioxo-1-oxa-3,6-diazacyclopentadec-11-ene-7,9-dicarboxamide B31. 100 mg of B31 was submitted to prep-HPLC purification to afford (4S,7S,E)-15-benzyl-4-isobutyl-N7-methoxy-N7,N9,N9-trimethyl-2,5-dioxo-1-oxa-3,6-diazacyclopentadec-11-ene-7,9-dicarboxamide B31.

TLC system: 10% Methanol in DCM; Rf: 0.4

LCMS (ESI): m/z 549.35 (M+H)⁺

### (4S,7S)-15-Benzyl-7-formyl-4-isobutyl-N,N-dimethyl-2,5-dioxo-1-oxa-3,6-diazacyclopentadec-11-ene-9-carboxamide B38

To a stirred solution of (4S,7S)-15-benzyl-4-isobutyl-N7-methoxy-N7,N9,N9-trimethyl-2,5-dioxo-1-oxa-3,6-diazacyclopentadec-11-ene-7,9-dicarboxamide B31 (80 mg, 0.147 mmol) in THE (2 mL) was added 1M LAH in THE (0.17 mL, 0.176 mmol) at −20° C. and stirred up to RT for 3 h. The progress of the reaction was monitored by TLC and LCMS. After 3 h, the reaction mixture was quenched with sat. ammonium chloride (2 mL), extracted with ethyl acetate (2×20 mL), washed with water (2×10 mL) and brine solution (10 mL). The combined organic layer was dried over anhydrous Na₂SO₄ and evaporated to afford crude residue. The crude was purified by prep HPLC to afforded (4S,7S)-15-benzyl-7-formyl-4-isobutyl-N,N-dimethyl-2,5-dioxo-1-oxa-3,6-diazacyclopentadec-11-ene-9-carboxamide B38.

TLC system: 5% Methanol in DCM; Rf: 0.4

LCMS (ESI): m/z 484.48 (M+H)⁻

### Diethyl (((4S,7S)-15-benzyl-9-(dimethylcarbamoyl)-4-isobutyl-2,5-dioxo-1-oxa-3,6-diazacyclopentadec-11-en-7-yl)(hydroxy)methyl)phosphonate B30

To a stirred solution of (4S,7S)-15-benzyl-7-formyl-4-isobutyl-N,N-dimethyl-2,5-dioxo-1-oxa-3,6-diazacyclopentadec-11-ene-9-carboxamide B38 (180 mg, 0.37 mmol) in DCM (3 mL) was added DIPEA (0.2 mL, 1.11 mmol) and diethyl phosphite (0.15 mL, 1.11 mmol). Then reaction mixture was stirred at RT for 16 h. The progress of the reaction was monitored by TLC and LCMS. After 16 h, the reaction mixture was diluted by DCM (20 mL), washed with water (2×30 mL) and brine solution. The organic layer was dried over anhydrous Na₂SO₄, and evaporated to afford crude residue. The crude was purified by prep HPLC to afford diethyl (((4S,7S)-15-benzyl-9-(dimethylcarbamoyl)-4-isobutyl-2,5-dioxo-1-oxa-3,6-diazacyclopentadec-11-en-7-yl)(hydroxy)methyl)phosphonate B30

TLC system: 10% Methanol in DCM; Rf: 0.35

LCMS (ESI): m/z 624.48 (M+H)⁺

### Synthesis of Compound B64 and B32-B37

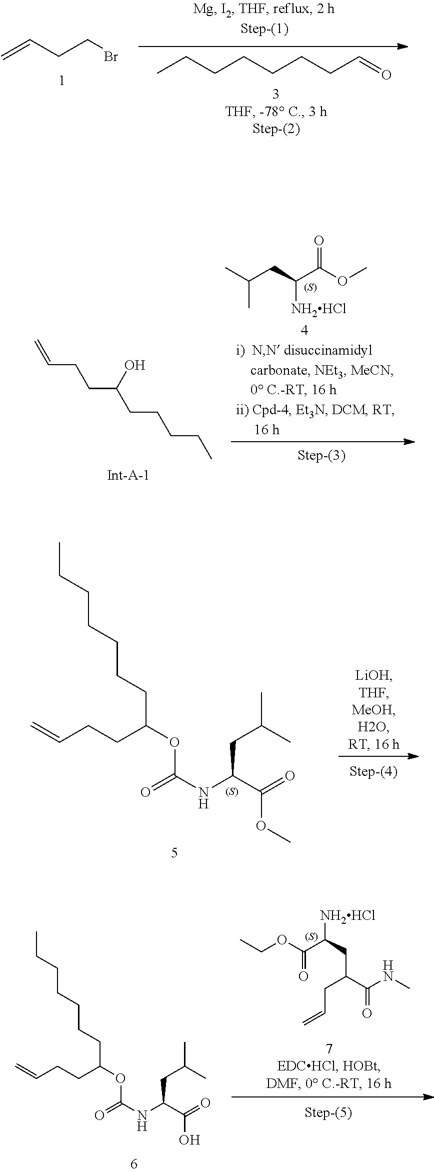

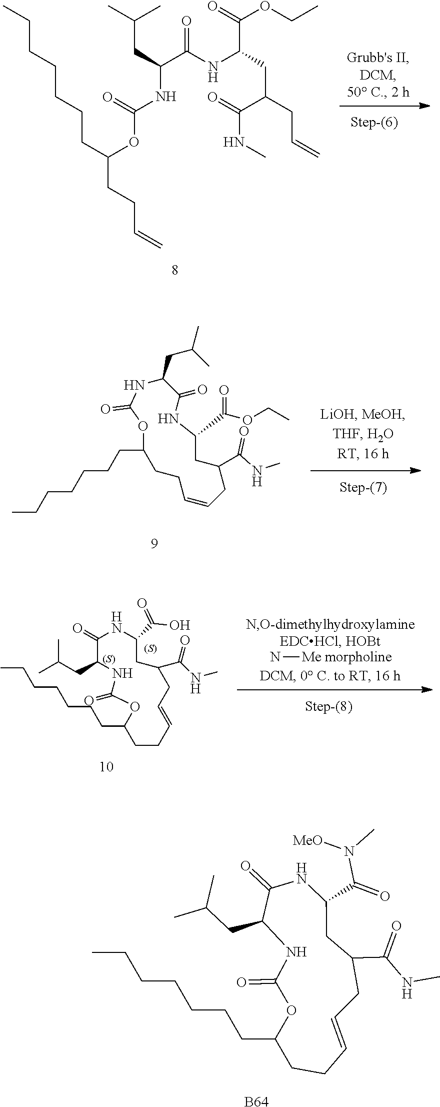

### Dodec-1-en-5-ol (Int-A-1)

To a dried magnesium turning (1.8 g, 78.12 mmol) and catalytic iodine (189 mg, 074 mmol) in a three-neck RBF added dry diethyl ether (100 mL), was slowly added 4-bromobut-1-ene (1) (10 g, 74.074 mmol) then 1,2-dibromo ethane (139 mg, 074 mmol) at RT. (Observed generation of exotherm and solvent reflux). Reaction mixture was stirred at RT for 2 h. The in situ generated heptylmagnesium bromide (2) (15.07 g, 74.626 mmol) has been used directly for the reaction. To a stirred solution of octanal (3) (12 g, 93.75 mmol) in dry diethyl ether (40 mL) at −78° C. was slowly added the freshly prepared heptylmagnesium bromide (2) (15.07 g, 74.626 mmol) drop wise. The reaction mixture was stirred at −78° C. for 2 h. The reaction mixture was quenched with sat. NH₄Cl (100 mL), extracted with EtOAc (3×250 mL), dried over anhydrous Na₂SO₄ and evaporated under vacuum. Obtained crude was purified by silica column eluting with 2% EtOAc and pet ether to afford dodec-1-en-5-ol Int-A-1.

### Methyl ((dodec-1-en-5-yloxy)carbonyl)-L-leucinate (5)

To a stirred solution of dodec-1-en-5-ol Int-A-1 (12 g, 65.57 mmol) in acetonitrile (120 mL) at 0° C. was added triethylamine (27 mL, 196.72 mmol) and N,N-disuccinamidyl carbonate (41.9 g, 163.93 mmol) and stirred at room temperature for 16 h. After 16 h, the reaction mixture was evaporated to dryness under reduced pressure. The crude was diluted with ice water (100 mL), extracted with EtOAc (2×55 mL), dried over anhydrous Na₂SO₄ and evaporated under reduced pressure to obtain crude residue (5 g, 15.38 mmol). It was taken in DCM (100 mL) added triethylamine (6.4 mL, 46.15 mmol)) and methyl-L-leucinate hydrochloride (4) (5.5 g, 30.76 mmol) at 0° C. and stirred at room temperature for 16 h. The progress of the reaction was monitored by TLC. The reaction mixture was quenched with ice water (50 mL) and extracted with DCM (2×50 mL). Organic layer was washed with brine solution (30 mL), dried over sodium sulfate and concentrated under reduced pressure. Crude was purified through silica gel (100-200 mesh) column chromatography by eluting with 1% EtOAc in pet ether to afford methyl ((dodec-1-en-5-yloxy)carbonyl)-L-leucinate (5).

### ((Dodec-1-en-5-yloxy)carbonyl)-L-leucine (6)

To a stirred solution of methyl ((dodec-1-en-5-yloxy)carbonyl)-L-leucinate (5) (5 g, 14.08 mmol) in dry THE (40 mL), methanol (20 mL) and water (10 mL), was added lithium hydroxide (1 g, 42.25 mmol) at 0° C. and stirred at room temperature for 16 h. The progress of the reaction was monitored by TLC. Reaction mixture distilled off under reduced pressure, crude compound acidified with 2N aq. HCl and adjusted pH ˜4, and extracted with EtOAc (2×200 mL), dried over anhydrous Na₂SO₄, and concentrated under reduced pressure to afford compound ((dodec-1-en-5-yloxy)carbonyl)-L-leucine (6).

### Ethyl (2S)-2-((2S)-2-(((dodec-1-en-5-yloxy)carbonyl)amino)-4-methylpentanamido)-4-(methylcarbamoyl)hept-6-enoate (8)

To a stirred solution of ((dodec-1-en-5-yloxy)carbonyl)-L-leucine (6) (2.5 g, 7.33 mmol) in DMF (35 mL), were added EDC.HCl (2.1 g, 10.99 mmol), HOBt (1.4 g, 10.99 mmol) and DIPEA (3.7 mL, 21.99 mmol) at 0° C. and stirred 10 min, was added ethyl (2S)-2-amino-4-(methylcarbamoyl)hept-6-enoate hydrochloride (7) (2.3 g, 8.78 mmol) at 0° C. and stirred at room temperature for 16 h. The progress of the reaction was monitored by TLC. Reaction mixture was diluted with ice water (150 mL), extracted with ethyl acetate (2×150 mL), dried over anhydrous Na₂SO₄ and evaporated under reduced pressure. The crude residue was purified by column Grace normal phase with eluent 60% EtOAc in pet ether to afford ethyl (2S)-2-((2S)-2-(((dodec-1-en-5-yloxy)carbonyl)amino)-4-methylpentanamido)-4-(methylcarbamoyl)hept-6-enoate (8).

### Ethyl (4S,7S)-15-heptyl-4-isobutyl-9-(methylcarbamoyl)-2,5-dioxo-1-oxa-3,6-diazacyclopentadec-11-ene-7-carboxylate (9)

To a stirred solution of ethyl (2S)-2-((2S)-2-(((dodec-1-en-5-yloxy)carbonyl)amino)-4-methylpentanamido)-4-(methylcarbamoyl)hept-6-enoate (8) (1.5 g, 2.722 mmol) in DCM (10 mL) was added Grubb's II catalyst (231 mg, 0.27 mmol) at room temperature and stirred at 50° C. for 2 h. The progress of the reaction was monitored by TLC. After 2 h, the reaction mixture was quenched with water (25 mL) and extracted with DCM (2×50 mL). Combined organic layer were washed with brine solution (30 mL), dried over anhydrous Na₂SO₄ and concentrated under reduced pressure to afford crude residue. It was purified by column Grace normal phase using of 4% methanol/DCM to afford ethyl (4S,7S,Z)-15-heptyl-4-isobutyl-9-(methylcarbamoyl)-2,5-dioxo-1-oxa-3,6-diazacyclopentadec-11-ene-7-carboxylate (9).

### (4S,7S)-15-Heptyl-4-isobutyl-9-(methylcarbamoyl)-2,5-dioxo-1-oxa-3,6-diazacyclopentadec-11-ene-7-carboxylic acid (10)

To a stirred solution of ethyl (4S,7S)-15-heptyl-4-isobutyl-9-(methylcarbamoyl)-2,5-dioxo-1-oxa-3,6-diazacyclopentadec-11-ene-7-carboxylate (9) (2 g, 3.82 mmol) in THE (10 mL), methanol (10 mL) and water (5 mL), was added lithium hydroxide (275 mg, 11.47 mmol) at room temperature and stirred at same for 16 h. Reaction mixture completely distilled under reduced pressure, crude compound acidified with 2N HCl solution up to pH ˜4, and extracted with ethyl acetate (2×55 mL), dried over anhydrous Na₂SO₄, concentrated under reduced pressure to afford compound (4S,7S)-15-heptyl-4-isobutyl-9-(methylcarbamoyl)-2,5-dioxo-1-oxa-3,6-diazacyclopentadec-11-ene-7-carboxylic acid (10).

### (4S,7S)-15-Heptyl-4-isobutyl-N⁷-methoxy-N⁷,N⁹-dimethyl-2,5-dioxo-1-oxa-3,6-diazacyclopentadec-11-ene-7,9-dicarboxamide B64

To a stirred solution of (4S,7S)-15-heptyl-4-isobutyl-9-(methylcarbamoyl)-2,5-dioxo-1-oxa-3,6-diazacyclopentadec-11-ene-7-carboxylic acid (10) (2 g, 4.04 mmol) in tetrahydrofuran (10 mL) and DCM (10 mL) added EDC.HCl (926 mg, 4.84 mmol), HOBT (659 mg, 4.84 mmol), N-methylmorpholine (0.5 mL, 4.84 mmol) and N,O-dimethylhydroxylamine (472 mg, 4.84 mmol) at 0° C. simultaneously and stirred at room temperature for 16 h. The progress of the reaction was monitored by TLC. After 16 h, the reaction mixture was diluted with ice water (100 mL), extracted with DCM (2×50 mL), dried over anhydrous Na₂SO₄ and evaporated under reduced pressure. The crude residue was purified by prep HPLC to (4S,7S)-15-heptyl-4-isobutyl-N⁷-methoxy-N⁷,N⁹-dimethyl-2,5-dioxo-1-oxa-3,6-diazacyclopentadec-11-ene-7,9-dicarboxamide B64, which was subjected to chiral separation to yield B32 to B37

TLC system: 10% Methanol/DCM; R_{f}: 0.4

LCMS (ESI): m/z 539.32 (M+H)⁺

### Chiral HPLC Purification of B32 to B37

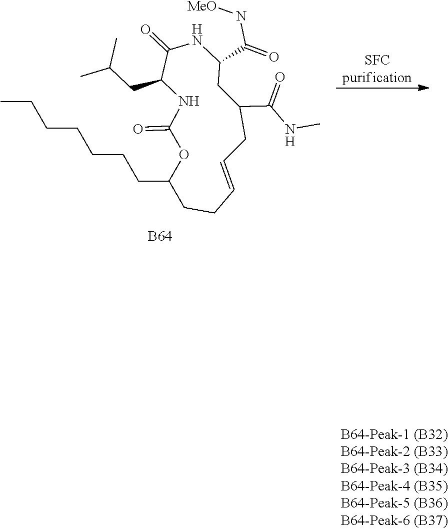 [SMILES: "B64" ; CCCCCCCC1CC/C=C/CC(C(=O)NC)C[C@@H](C(=O)NOC)NC(=O)[C@H](CC(C)C)NC(=O)O1]

SFC purification of 400 mg by Preparative SFC Conditions Column/dimensions: CHIRALPAK-IC (30×250 mm), 5μ % CO₂: 85% % Co solvent: 15% (0.2% 7M methanolic ammonia in methanol), Total Flow: 95.0 g/min Back Pressure: 100 bar Temperature: 35 OC UV: MAX PLOT Solubility: Methanol

### Synthesis of Compound B42

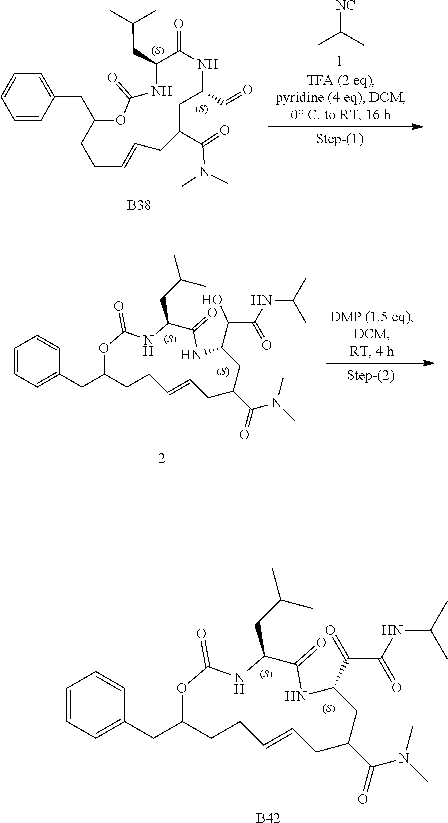

### (4S,7S)-15-benzyl-7-(1-hydroxy-2-(isopropylamino)-2-oxoethyl)-4-isobutyl-N,N-dimethyl-2,5-dioxo-1-oxa-3,6-diazacyclopentadec-11-ene-9-carboxamide (2)

To the stirred solution of (4S,7S)-15-benzyl-7-formyl-4-isobutyl-N,N-dimethyl-2,5-dioxo-1-oxa-3,6-diazacyclopentadec-11-ene-9-carboxamide B38 (380 mg, 0.783 mmol) in DCM (5 mL) was added pyridine (0.25 mL, 3.032 mmol) and 2-isocyanopropane (0.12 mL, 1.567 mmol). Then after 5 min added TFA (0.04 mL, 1.567 mmol) at 0° C. and reaction mixture was stirred at RT for 16 h. The progress of the reaction was monitored by TLC and LCMS. After 16 h, the reaction mixture was quenched with aq. 1N HCl (4 mL) and extracted with DCM (2×30 mL) and washed with brine solution (20 mL). Organic layer was dried over anhydrous Na₂SO₄, and evaporated to afford crude (4S,7S)-15-benzyl-7-(1-hydroxy-2-(isopropylamino)-2-oxoethyl)-4-isobutyl-N,N-dimethyl-2,5-dioxo-1-oxa-3,6-diazacyclopentadec-11-ene-9-carboxamide 2

### (4S,7S)-15-benzyl-4-isobutyl-7-(2-(isopropylamino)-2-oxoacetyl)-N,N-dimethyl-2,5-dioxo-1-oxa-3,6-diazacyclopentadec-11-ene-9-carboxamide B42

To a stirred solution of (4S,7S)-15-benzyl-7-(1-hydroxy-2-(isopropylamino)-2-oxoethyl)-4-isobutyl-N,N-dimethyl-2,5-dioxo-1-oxa-3,6-diazacyclopentadec-11-ene-9-carboxamide (2) (250 mg, 0.437 mmol) in DCM (4 mL) was added Dess-Martin periodinane (167 mg, 0.655 mmol) at RT for 4 h. The progress of the reaction was monitored by TLC and LCMS. After 4 h, reaction mixture was diluted with DCM (20 mL), washed with water (2×30 mL) and brine solution (20 mL). Organic layer was dried over anhydrous Na₂SO₄, and evaporated to afford crude residue. Crude residue was purified by prep HPLC and afforded (4S,7S)-15-benzyl-4-isobutyl-7-(2-(isopropylamino)-2-oxoacetyl)-N,N-dimethyl-2,5-dioxo-1-oxa-3,6-diazacyclopentadec-11-ene-9-carboxamide B42

TLC system: 10% Methanol in DCM; Rf: 0.1

LCMS (ESI): m/z 571.58 (M+H)⁺

### Synthesis of B44, B45, and B43

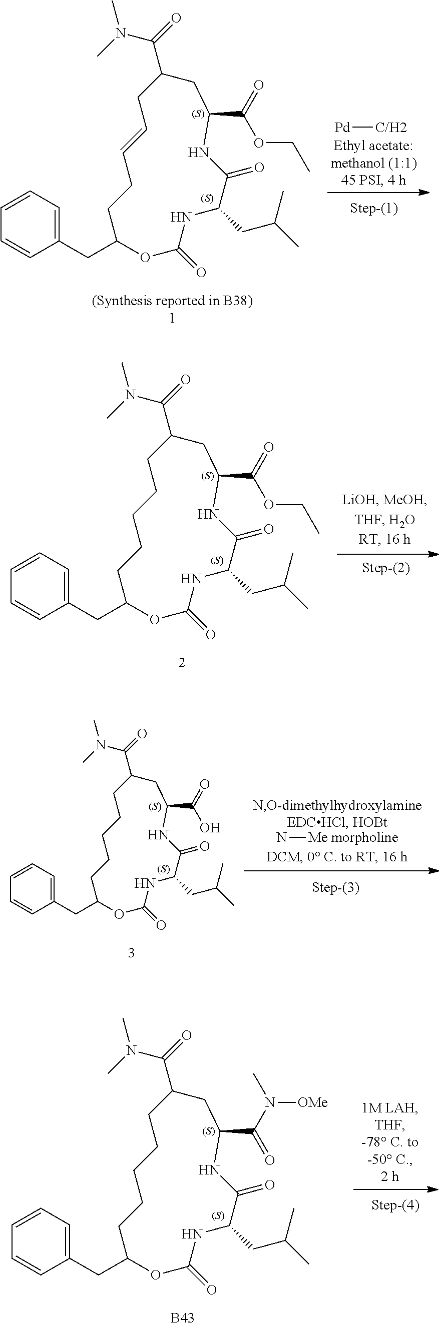

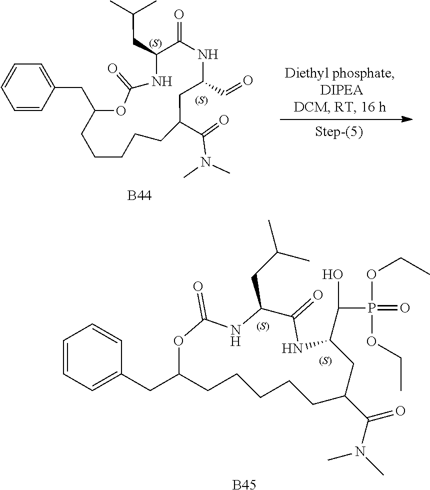

### Ethyl (4S,7S)-15-benzyl-9-(dimethylcarbamoyl)-4-isobutyl-2,5-dioxo-1-oxa-3,6-diazacyclopentadecane-7-carboxylate (1)

To a stirred solution of ethyl (4S,7S)-15-benzyl-9-(dimethylcarbamoyl)-4-isobutyl-2,5-dioxo-1-oxa-3,6-diazacyclopentadec-11-ene-7-carboxylate (1) (1.6 g, 3.024 mmol) in methanol (25 mL) and ethyl acetate (25 mL) was treated with 10% w/w of 10% Pd/carbon (160 mg) and stirred under hydrogen atmosphere at 45 psi in parashaker for 4 h. After 4 h, the reaction mixture was filtered through celite using methanol (2×50 mL) and concentrated under reduced pressure. Crude compound was purified through combi flash column chromatography using 5% methanol in dichloromethane to afford ethyl (4S,7S)-15-benzyl-9-(dimethylcarbamoyl)-4-isobutyl-2,5-dioxo-1-oxa-3,6-diazacyclopentadecane-7-carboxylate (2).

### (4S,7S)-15-benzyl-9-(dimethylcarbamoyl)-4-isobutyl-2,5-dioxo-1-oxa-3,6-diazacyclopentadecane-7-carboxylic acid (2)

To a stirred solution of ethyl (4S,7S)-15-benzyl-9-(dimethylcarbamoyl)-4-isobutyl-2,5-dioxo-1-oxa-3,6-diazacyclopentadecane-7-carboxylate 2 (1.6 g, 3.024 mmol) in THF (8 mL), MeOH (4 mL) and H₂O (2 mL) was added LiOH (0.29 g, 12.098 mmol) at rt for 16 h. Reaction mixture was distilled off under reduced pressure, crude compound acidified to pH ˜4 with 2N HCl and extracted with EtOAc (2×100 mL), dried over anhydrous Na₂SO₄, concentrated under reduced pressure to afford (4S,7S)-15-benzyl-9-(dimethylcarbamoyl)-4-isobutyl-2,5-dioxo-1-oxa-3,6-diazacyclopentadecane-7-carboxylic acid 3.

### (4S,7S)-15-benzyl-4-isobutyl-N7-methoxy-N7,N9,N9-trimethyl-2,5-dioxo-1-oxa-3,6-diazacyclopentadecane-7,9-dicarboxamide B43

To a stirred solution of (4S,7S)-15-benzyl-9-(dimethylcarbamoyl)-4-isobutyl-2,5-dioxo-1-oxa-3,6-diazacyclopentadecane-7-carboxylic acid 3 (150 mg, 0.298 mmol) in THE (4 mL) and DCM (4 mL) were added N-methylmorpholine (0.13 mL, 0.894 mmol), EDC.HCl (85 mg, 0.447 mmol) and HOBT (60 mg, 0.447 mmol) at 0° C. and for 10 min, was added N,O-dimethylhydroxylamine (43 mg, 0.447 mmol) at 0° C. and stirred at room temperature for 16 h. Reaction mixture was diluted with ice water (20 mL), extracted with EtOAc (2×20 mL), dried over anhydrous Na₂SO₄ and evaporated under reduced pressure. The crude residue was purified by Grace reverse phase by eluting with 40% 0.1% Formic acid in water and acetonitrile to afford crude, which was purified by prep-HPLC purification (4S,7S)-15-benzyl-4-isobutyl-N7-methoxy-N7,N9,N9-trimethyl-2,5-dioxo-1-oxa-3,6-diazacyclopentadecane-7,9-dicarboxamide B43.

TLC system: 10% Methanol in DCM; Rf: 0.4

LCMS (ESI): m/z 547.3 (M+H)⁺

### (4S,7S)-15-benzyl-7-formyl-4-isobutyl-N,N-dimethyl-2,5-dioxo-1-oxa-3,6-diazacyclopentadecane-9-carboxamide B44

To a stirred solution of (4S,7S)-15-benzyl-4-isobutyl-N7-methoxy-N7,N9,N9-trimethyl-2,5-dioxo-1-oxa-3,6-diazacyclopentadecane-7,9-dicarboxamide B43 (150 mg, 0.274 mmol) in THE (2 mL) was added 2.4M LAH in THE (0.17 mL, 0.41 mmol) at −78° C. and stirred up to −50° C. for 2 h. Reaction was monitoring by TLC. Reaction mixture was quenched with sat. ammonium chloride solution (10 mL), extracted with ethyl acetate (2×20 mL), washed with water (2×10 mL) and brine solution (10 mL). Organic layer was dried over anhydrous Na₂SO₄ and evaporated to afford crude residue. It was purified by prep HPLC to afforded (4S,7S)-15-benzyl-7-formyl-4-isobutyl-N,N-dimethyl-2,5-dioxo-1-oxa-3,6-diazacyclopentadecane-9-carboxamide B44

TLC system: 5% Methanol in DCM; Rf: 0.3

LCMS (ESI): m/z 488.63 (M+H)⁺

### Diethyl (((4S,7S)-15-benzyl-9-(dimethylcarbamoyl)-4-isobutyl-2,5-dioxo-1-oxa-3,6-diazacyclopentadecan-7-yl)(hydroxy)methyl)phosphonate B45

To a stirred solution of (4S,7S)-15-benzyl-7-formyl-4-isobutyl-N,N-dimethyl-2,5-dioxo-1-oxa-3,6-diazacyclopentadecane-9-carboxamide B44 (280 mg, 0.574 mmol) in DCM (4 mL) was added DIPEA (0.3 mL, 1.72 mmol) and diethylphosphite (0.26 mL, 1.72 mmol). Then reaction mixture was stirred at RT for 16 h. Reaction mixture was taken in to DCM (20 mL), washed with water (2×30 mL) and brine solution. Organic layer was dried over anhydrous Na₂SO₄, and evaporated to afford crude residue. It was purified by prep HPLC and afforded Diethyl (((4S,7S)-15-benzyl-9-(dimethylcarbamoyl)-4-isobutyl-2,5-dioxo-1-oxa-3,6-diazacyclopentadecan-7-yl)(hydroxy) methyl)phosphonate B45

TLC system: 10% Methanol in DCM; Rf: 0.4

LCMS (ESI): m/z 626.70 (M+H)⁺

LCMS (ESI): m/z 401.43 (M+H)⁺

### Synthesis of Compounds A114 and A116

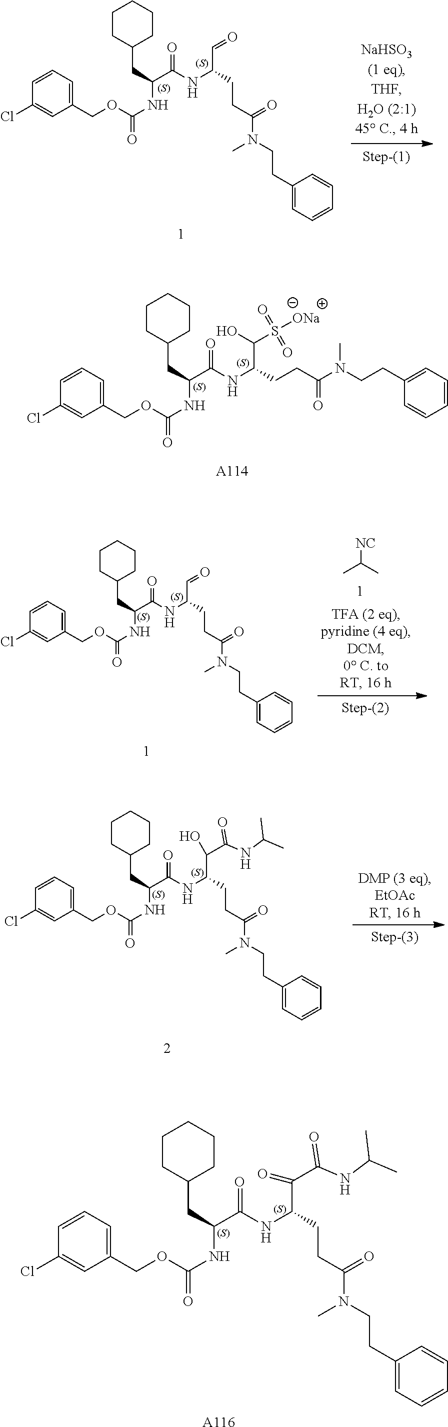

### Sodium (2S)-2-((S)-2-((((3-chlorobenzyl) oxy) carbonyl) amino)-3-cyclohexylpropanamido)-1-hydroxy-5-(methyl (phenethyl) amino)-5-oxopentane-1-sulfonate (Compound A114)

To a stirred solution of 3-chlorobenzyl ((S)-3-cyclohexyl-1-(((S)-5-(methyl(phenethyl)amino)-1,5-dioxopentan-2-yl)amino)-1-oxopropan-2-yl)carbamate (1) (120 mg, 0.210 mmol) was dissolved in THF/H₂O (4/1) (2.5 mL) was added NaHSO₃ (21.93 mg, 0.210 mmol) at 0° C. and stirred at 45° C. for 4 h. Reaction mixture was cooled to RT and concentrated to get crude compound. It was purified by trituration of ethyl acetate in n-pentane for 5 times at −78° C., DCM in n-pentane for 4 times at −78° C., washed with n-pentane for 3 times afforded pure sodium (2S)-2-((S)-2-((((3-chlorobenzyl)oxy)carbonyl)amino)-3-cyclohexylpropanamido)-1-hydroxy-5-(methyl(phenethyl)amino)-5-oxopentane-1-sulfonate (Compound A114) TLC system: 15% MeOH/DCM Rf: 0.3 LCMS (ESI): m/z 652.42[M+H]+M-Na

### 3-Chlorobenzyl ((2S)-3-cyclohexyl-1-(((3S)-2-hydroxy-1-(isopropylamino)-6-(methyl(phenethyl)amino)-1,6-dioxohexan-3-yl)amino)-1-oxopropan-2-yl)carbamate (2)

To a stirred solution of 3-chlorobenzyl ((S)-3-cyclohexyl-1-(((S)-5-(methyl(phenethyl)amino)-1,5-dioxopentan-2-yl)amino)-1-oxopropan-2-yl)carbamate (1) (700 mg, 1.230 mmol) was dissolved in DCM (15 mL) added Pyridine (0.3 mL, 4.92 mmol), 2-isocyanopropane (1) (145 mg, 2460 mmol) sequentially at 0° C. stirred for 10 min added TFA (0.1 mL, 2.460 mmol) at 0° C. and stirred at RT for 16 h. The progress of the reaction was monitored by TLC and LCMS. Reaction mixture was quenched with ice water (100 mL), extracted with EtOAc (2×55 mL), the combined organic layers was washed with 1N HCl (3×20 mL) brine solution (3×20 mL) organic layers was dried over anhydrous Na₂SO₄ and evaporated under reduced pressure. The crude residue 3-chlorobenzyl((2S)-3-cyclohexyl-1-(((3S)-2-hydroxy-1-(isopropylamino)-6-(methyl(phenethyl)amino)-1,6-dioxohexan-3-yl)amino)-1-oxopropan-2-yl)carbamate (2)

TLC system: 100% Ethyl acetate Rf: 0.3 LCMS (ESI): m/z 657.66[M+H]

### 3-Chlorobenzyl ((S)-3-cyclohexyl-1-(((S)-1-(isopropylamino)-6- (methyl (phenethyl) amino)-1, 2, 6-trioxohexan-3-yl) amino)-1-oxopropan-2-yl)carbamate (Compound A116)

To a stirred solution of 3-chlorobenzyl((2S)-3-cyclohexyl-1-(((3S)-2-hydroxy-1-(isopropyl amino)-6-(methyl(phenethyl)amino)-1,6-dioxohexan-3-yl)amino)-1-oxopropan-2-yl)carbamate (2) (270 mg, 0.411 mmol) in EtOAc (10 mL) at 0° C. was added Dess-Martin periodinane (523 mg, 1.23 mmol) and stirred at RT for 16 h. The progress of the reaction was monitored by TLC and LCMS. Reaction mixture was filter through celite pad and washed with Ethyl acetate (25 mL) and filtrate was washed with hypo solution (3×20 mL) followed by saturated NaHCO₃ solution (3×20 mL). Organic layer was dried over anhydrous Na₂SO₄, filtered and concentrated to get crude residue. It was purified by prep HPLC to afford 3-chlorobenzyl ((S)-3-cyclohexyl-1-(((S)-1-(isopropylamino)-6-(methyl(phenethyl)amino)-1,2,6-trioxohexan-3-yl)amino)-1-oxopropan-2-yl)carbamate (Compound A116). TLC system: 100% Ethyl acetate Rf: 0.4

LCMS (ESI): m/z 655.49. (M+H)⁺

### Synthesis of Compounds A70 and A59

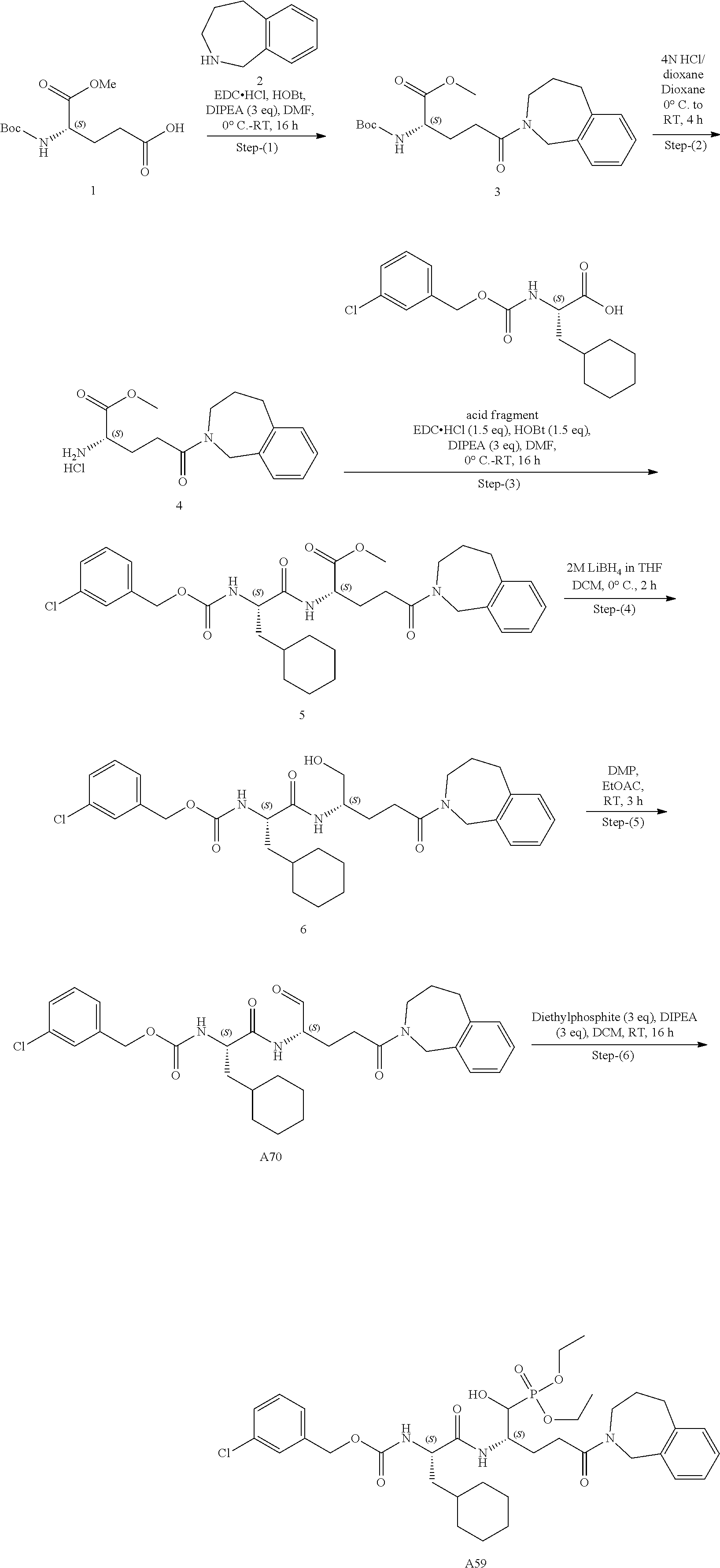

### Methyl (S)-2-((tert-butoxycarbonyl)amino)-5-oxo-5-(1,3,4,5-tetrahydro-2H-benzo[c]azepin-2-yl)pentanoate (3)

At 0° C., to a stirred solution of (S)-4-((tert-butoxycarbonyl)amino)-5-methoxy-5-oxopentanoic acid (1) (2 g, 7.66 mmol) in DMF (20 mL) was added EDC.HCl (2.1 g, 11.49 mmol), HOBT (1.5 g, 11.49 mmol), DIPEA (4 mL, 22.98 mmol) and 2,3,4,5-tetrahydro-1H-benzo[c]azepine (2) (1.2 g, 8.429 mmol) sequentially then stirred at room temperature for 16 h. The progress of the reaction was monitored by TLC and LCMS. Reaction mixture was diluted with ice water (100 mL), extracted with EtOAc (2×55 mL), dried over anhydrous Na₂SO₄ and evaporated under reduced pressure. The crude residue was purified by silica gel column (combiflash normal phase) using 60% EtOAc in pet ether to afford methyl (S)-2-((tert-butoxycarbonyl) amino)-5-oxo-5-(1,3,4,5-tetrahydro-2H-benzo[c]azepin-2-yl)pentanoate (3). TLC system: 70% EtOAc in pet ether Rf: 0.3 LCMS (ESI): m/z 391.58 [M+H]⁺

### Methyl (S)-2-amino-5-oxo-5-(1,3,4,5-tetrahydro-2H-benzo[c]azepin-2-yl)pentanoate hydrochloride (4)

To a stirred solution of methyl (S)-2-((tert-butoxycarbonyl) amino)-5-oxo-5-(1,3,4,5-tetrahydro-2H-benzo[c]azepin-2-yl)pentanoate (3) (1.5 g, 3.84 mmol) in 1,4-dioxane (10 mL) was added 4M HCl in dioxane (15 mL) drop wise at 0° C. and stirred at RT for 4 h. The progress of the reaction was monitored by TLC. After consumption of starting material, the reaction mixture was evaporated under reduced pressure followed by trituration with n-pentane to afford methyl(S)-2-amino-5-oxo-5-(1,3,4,5-tetrahydro-2H-benzo[c]azepin-2-yl)pentanoate hydrochloride (4) which was directly used in the next step. TLC system: 100% EtOAc Rf: 0.1 LCMS (ESI): m/z 291.35 (M+H)⁺

### Methyl (S)-2-((S)-2-((((3-chlorobenzyl)oxy)carbonyl)amino)-3-cyclohexylpropanamido)-5-oxo-5-(1,3,4,5-tetrahydro-2H-benzo[c]azepin-2-yl)pentanoate (5)

To a stirred solution of (S)-2-((((3-chlorobenzyl)oxy)carbonyl)amino)-3-cyclohexyl propanoic acid (1 g, 2.949 mmol) in DMF (10 mL) at 0° C. was added EDC.HCl (845 mg, 3.539 mmol), HOBT (570 mg, 4.424 mmol), DIPEA (2 mL, 8.456 mmol) and methyl(S)-2-amino-5-oxo-5-(1,3,4,5-tetrahydro-2H-benzo[c]azepin-2-yl)pentanoate hydrochloride (1.026 g, 3.533 mmol) simultaneously and stirred at room temperature for 16 h. The progress of the reaction was monitored by TLC and LCMS. After 16 h, the reaction mixture was quenched with ice water (60 mL) and extracted with ethyl acetate (2×30 mL), dried over sodium sulfate and evaporated under reduced pressure. The crude residue was purified by silica gel column using grace NP by eluting with 3% methanol in DCM to afford methyl (S)-2-((S)-2-((((3-chlorobenzyl) oxy) carbonyl)amino)-3-cyclohexylpropanamido)-5-oxo-5-(1,3,4,5-tetrahydro-2H-benzo[c]azepin-2-yl)pentanoate (5). TLC system: 60% Ethyl acetate in pet-ether Rf: 0.4 LCMS (ESI): m/z 612.21 [M+H]

### 3-Chlorobenzyl ((S)-3-cyclohexyl-1-(((S)-1-hydroxy-5-oxo-5-(1,3,4,5-tetrahydro-2H-benzo[c]azepin-2-yl)pentan-2-yl)amino)-1-oxopropan-2-yl)carbamate (6)

To a stirred solution of methyl (S)-2-((S)-2-((((3-chlorobenzyl) oxy) carbonyl)amino)-3-cyclohexylpropanamido)-5-oxo-5-(1,3,4,5-tetrahydro-2H-benzo[c]azepin-2-yl)pentanoate (5) (500 mg, 0.818 mmol) in DCM (5 mL) at 0° C. was added 2M LiBH₄ in THE (1.2 mL, 2.45 mmol) and reaction mixture stirred at RT for 2 h. The progress of the reaction was monitored by TLC and LCMS. Then the reaction mixture was quenched with ammonium chloride solution (15 mL) and extracted with DCM (2×20 mL). Combined organic layer was washed with brine solution (20 mL), dried over Na₂SO₄ and concentrated to get crude residue. It was purified by silica gel column chromatography to afford 3-chlorobenzyl((S)-3-cyclohexyl-1-(((S)-1-hydroxy-5-oxo-5-(1,3,4,5-tetrahydro-2H-benzo[c] azepin-2-yl)pentan-2-yl)amino)-1-oxopropan-2-yl)carbamate TLC system: 100% Ethyl acetate Rf: 0.2 LCMS (ESI): m/z 584.68 (M+H)⁺

### 3-Chlorobenzyl ((S)-3-cyclohexyl-1-(((S)-1,5-dioxo-5-(1,3,4,5-tetrahydro-2H-benzo[c]azepin-2-yl)pentan-2-yl)amino)-1-oxopropan-2-yl)carbamate (Compound A70)

To a stirred solution of 3-chlorobenzyl((S)-3-cyclohexyl-1-(((S)-1-hydroxy-5-oxo-5-(1,3,4,5-tetrahydro-2H-benzo[c]azepin-2-yl)pentan-2-yl)amino)-1-oxopropan-2-yl)carbamate (6) (100 mg, 0.171 mmol) in EtOAc (5 mL) at 0° C. was added Dess-Martin periodinane (218 mg, 0.514 mmol) and stirred at RT for 3 h. The progress of the reaction was monitored by TLC and LCMS. Reaction mixture was filter through celite pad and washed with Ethyl acetate (25 mL) and filtrate was washed with hypo solution (3×20 mL) followed by saturated NaHCO₃ solution (3×20 mL). Organic layer was dried over anhydrous Na₂SO₄, filtered and concentrated to get crude residue. It was purified by prep HPLC to afford 3-chlorobenzyl ((S)-3-cyclohexyl-1-(((S)-1,5-dioxo-5-(1,3,4,5-tetrahydro-2H-benzo[c]azepin-2-yl)pentan-2-yl)amino)-1-oxopropan-2-yl)carbamate (Compound A70). TLC system: 10% MeOH in DCM Rf: 0.4 LCMS (ESI): m/z 582.42. (M+H)⁺

### 3-Chlorobenzyl ((2S)-3-cyclohexyl-1-(((2S)-1-(diethoxyphosphoryl)-1-hydroxy-5-oxo-5-(1,3,4,5-tetrahydro-2H-benzo[c]azepin-2-yl)pentan-2-yl)amino)-1-oxopropan-2-yl)carbamate (Compound A59)

To a stirred solution of 3-chlorobenzyl ((S)-3-cyclohexyl-1-(((S)-1,5-dioxo-5-(1,3,4,5-tetrahydro-2H-benzo[c]azepin-2-yl)pentan-2-yl)amino)-1-oxopropan-2-yl)carbamate (130 mg, 0.223 mmol) in DCM (5 mL) at 0° C. was added DIPEA (0.123 mL, 0.671 mmol) followed by diethylphosphite (92 mg, 0.669 mmol) then stirred at RT for 16 h. The progress of the reaction was monitored by TLC and LCMS. After 16 h, the reaction mixture was quenched with water (15 mL) and extracted with DCM (2×20 mL). Combined organic layer was dried over anhydrous Na₂SO₄, and evaporated to afford crude residue. It was purified prep HPLC to afford 3-chlorobenzyl ((2S)-3-cyclohexyl-1-(((2S)-1-(diethoxyphosphoryl)-1-hydroxy-5-oxo-5-(1,3,4,5-tetrahydro-2H-benzo[c]azepin-2-yl)pentan-2-yl)amino)-1-oxopropan-2-yl)carbamate (Compound A59). TLC system: 60% Ethyl acetate in pet-ether Rf: 0.4 LCMS (ESI): m/z 720.5 (M+H)⁺

### Compounds A118 and A60

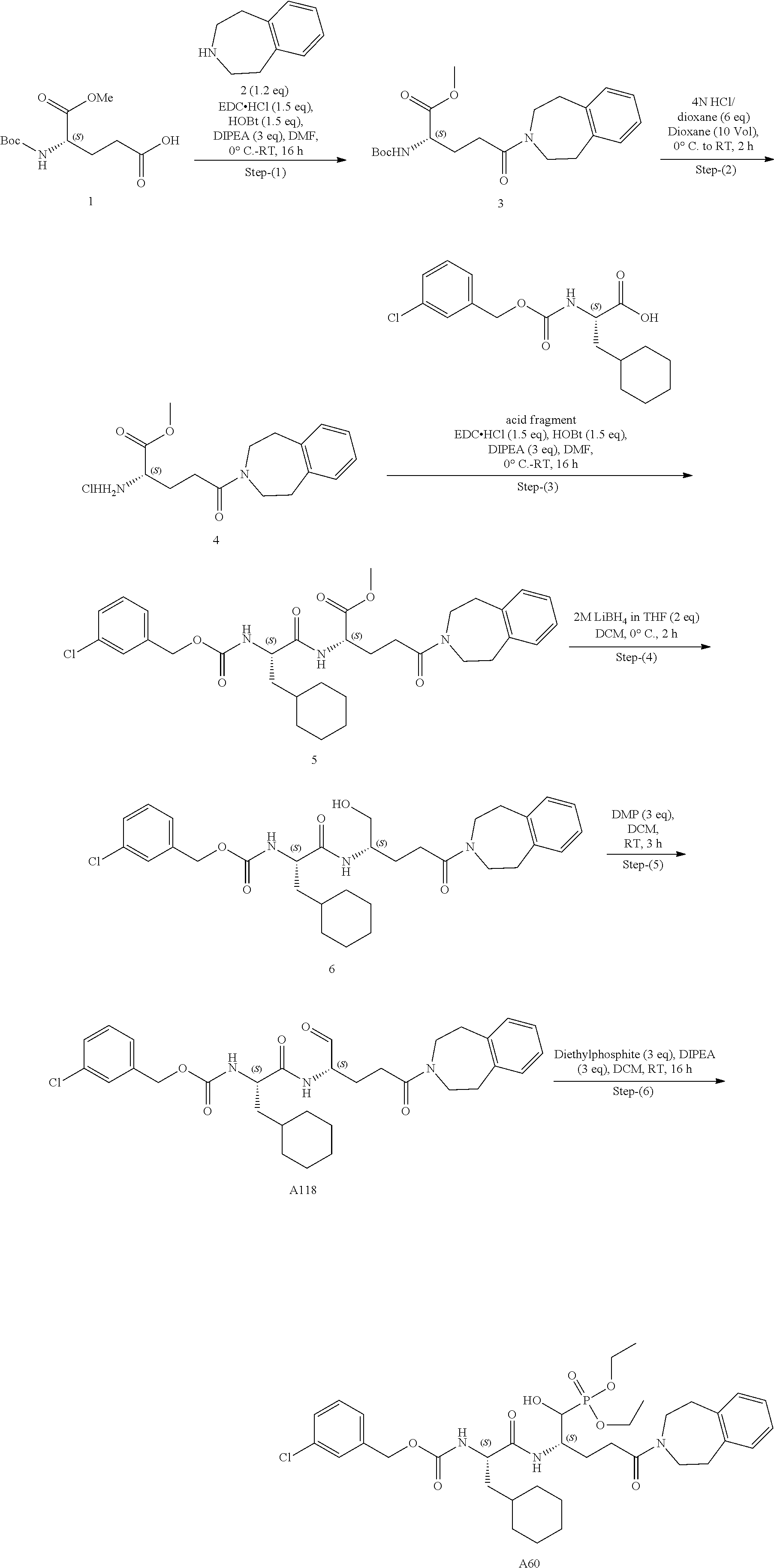

### Methyl (S)-2-((tert-butoxycarbonyl)amino)-5-oxo-5-(1,2,4,5-tetrahydro-3H-benzo[d]azepin-3-yl)pentanoate (3)

To a stirred solution of (S)-4-((tert-butoxycarbonyl)amino)-5-methoxy-5-oxopentanoic acid (1) (2 g, 7.662 mmol) in DMF (20 mL) were added EDC.HCl (2.19 g, 11.494 mmol), HOBt (0.716 g, 11.494 mmol), DIPEA (4.2 mL, 22.988 mmol) and 2,3,4,5-tetrahydro-1H-benzo[d]azepine (2) (1.35 g, 9.195 mmol) at 0° C. simultaneously and stirred at room temperature for 16 h. The progress of the reaction was monitored by TLC and LCMS. After 16 h, the reaction mixture was diluted with ice water (50 mL), extracted with ethyl acetate (2×50 mL). The combined organic layer was dried over sodium sulfate, filtered and evaporated under reduced pressure. The crude residue was purified by combi-flash compound eluted at 40% Ethyl acetate in pet ether to afford methyl (S)-2-((tert-butoxycarbonyl)amino)-5-oxo-5-(1,2,4,5-tetrahydro-3H-benzo[d]azepin-3-yl)pentanoate (3). TLC system: 50% Ethyl acetate in Pet ether Rf: 0.6

LCMS (ESI): m/z 391.58 [M+H]⁺

### Methyl (S)-2-amino-5-oxo-5-(1,2,4,5-tetrahydro-3H-benzo[d]azepin-3-yl)pentanoate hydrochloride (4)

To a stirred solution of methyl (S)-2-((tert-butoxycarbonyl)amino)-5-oxo-5-(1,2,4,5-tetrahydro-3H-benzo[d]azepin-3-yl)pentanoate (3) (1.8 g, 4.615 mmol) in 1,4-dioxane (20 mL) was added 4N HCl in dioxane (20 mL) with drop wise at 0° C. and the reaction mixture was stirred at RT for 2 h. The progress of the reaction was monitored by TLC and LCMS. After consumption of starting material, the reaction mixture was evaporated under reduced pressure to obtained crude compound, the resulting crude triturated with diethyl ether to afford methyl (S)-2-amino-5-oxo-5-(1,2,4,5-tetrahydro-3H-benzo[d]azepin-3-yl)pentanoate hydrochloride (4). TLC system: 10% Methanol in DCM Rf: 0.2

LCMS (ESI): m/z 291.28 [M+H]⁺

### Methyl (S)-2-((S)-2-((((3-chlorobenzyl)oxy)carbonyl)amino)-3-cyclohexylpropanamido)-5-oxo-5-(1,2,4,5-tetrahydro-3H-benzo[d]azepin-3-yl)pentanoate (5)

To a stirred solution of (S)-2-((((3-chlorobenzyl)oxy)carbonyl)amino)-3-cyclohexylpropanoic acid (acid fragment) (1.1 g, 3.244 mmol) DMF (15 mL) added EDC.HCl (1.23 g, 6.489 mmol), HOBT (0.716 g, 6.489 mmol), DIPEA (1.8 mL, 9.734 mmol) and methyl (S)-2-amino-5-oxo-5-(1,2,4,5-tetrahydro-3H-benzo[d]azepin-3-yl)pentanoate hydrochloride (4) (1.13 g, 3.893 mmol) at 0° C. simultaneously and stirred at room temperature for 16 h. The progress of the reaction was monitored by TLC and LCMS. After 16 h, reaction mixture was diluted with ice water (30 mL), extracted with ethyl acetate (2×50 mL). The combined organic layer was dried over sodium sulfate, filtered and evaporated under reduced pressure. The crude residue was purified by combi-flash, compound eluted at 60% Ethyl acetate in pet ether to afford methyl (S)-2-((S)-2-((((3-chlorobenzyl)oxy)carbonyl)amino)-3-cyclohexylpropanamido)-5-oxo-5-(1,2,4,5-tetrahydro-3H-benzo[d]azepin-3-yl)pentanoate (5). TLC system: 50% Ethyl acetate in Pet ether Rf: 0.5 LCMS (ESI): m/z 612.61 [M+H]⁺

### 3-Chlorobenzyl ((S)-3-cyclohexyl-1-(((S)-1-hydroxy-5-oxo-5-(1,2,4,5-tetrahydro-3H-benzo[d]azepin-3-yl)pentan-2-yl)amino)-1-oxopropan-2-yl)carbamate (6)

To a stirred solution of methyl (S)-2-((S)-2-((((3-chlorobenzyl)oxy)carbonyl)amino)-3-cyclohexylpropanamido)-5-oxo-5-(1,2,4,5-tetrahydro-3H-benzo[d]azepin-3-yl)pentanoate (5) (500 mg, 0.818 mmol) in THE (15 mL) was added 2M LiBH₄ in THE (0.8 mL, 1.636 mmol) at 0° C. and the reaction mixture stirred for 2 h at 0° C. The progress of the reaction was monitored by TLC and LCMS. Then reaction mixture was quenched with water (10 mL) and extracted with ethyl acetate (2×30 mL). Organic layer was washed with brine solution (10 mL), dried over Na₂SO₄ and concentrated to get crude compound. It was purified combi-flash, compound eluted at 80% Ethyl acetate in pet ether to afford 3-chlorobenzyl ((S)-3-cyclohexyl-1-(((S)-1-hydroxy-5-oxo-5-(1,2,4,5-tetrahydro-3H-benzo[d]azepin-3-yl)pentan-2-yl)amino)-1-oxopropan-2-yl)carbamate (6). TLC system: 80% Ethyl acetate in Pet ether Rf: 0.2 LCMS (ESI): m/z 584.59 (M+H)⁺

### 3-Chlorobenzyl ((S)-3-cyclohexyl-1-(((S)-1,5-dioxo-5-(1,2,4,5-tetrahydro-3H-benzo[d]azepin-3-yl)pentan-2-yl)amino)-1-oxopropan-2-yl)carbamate (Compound A118)

To a stirred solution of 3-chlorobenzyl ((S)-3-cyclohexyl-1-(((S)-1-hydroxy-5-oxo-5-(1,2,4,5-tetrahydro-3H-benzo[d]azepin-3-yl)pentan-2-yl)amino)-1-oxopropan-2-yl)carbamate (6) (200 mg, 0.342 mmol) was dissolved in DCM (10 mL) was added Dess-Martin periodinane (290 mg, 0.684 mmol) at 0° C. and stirred at RT for 3 h. Reaction mixture was diluted with DCM (20 mL) and washed with sat. Hypo solution (3×10 mL), sat. NaHCO₃ solution (3×20 mL). Organic layer was dried over anhydrous Na₂SO₄, filtered and concentrated to afford 3-chlorobenzyl ((S)-3-cyclohexyl-1-(((S)-1,5-dioxo-5-(1,2,4,5-tetrahydro-3H-benzo[d]azepin-3-yl)pentan-2-yl)amino)-1-oxopropan-2-yl)carbamate(Compound A118). TLC system: 80% Ethyl acetate in Pet ether Rf: 0.4 LCMS (ESI): m/z 582.52 (M+H)⁺

### 3-Chlorobenzyl ((2S)-3-cyclohexyl-1-(((2S)-1-(diethoxyphosphoryl)-1-hydroxy-5-oxo-5-(1,2,4,5-tetrahydro-3H-benzo[d]azepin-3-yl)pentan-2-yl)amino)-1-oxopropan-2-yl)carbamate (Compound A60)

To a stirred solution of 3-chlorobenzyl ((S)-3-cyclohexyl-1-(((S)-1,5-dioxo-5-(1,2,4,5-tetrahydro-3H-benzo[d]azepin-3-yl)pentan-2-yl)amino)-1-oxopropan-2-yl)carbamate (Compound A118) (250 mg crude, 0.429 mmol) in DCM (10 mL) added DIPEA (0.2 mL, 1.288 mmol) followed by added diethyl phosphite (0.1 mL, 0.859 mmol) and the reaction mixture stirred at RT for 16 h. The progress of the reaction was monitored by TLC and LCMS. After 16 h, the reaction mixture quenched with ammonium chloride (15 mL) and extracted with DCM (2×20 mL). Combined organic layer was dried over anhydrous Na₂SO₄, and evaporated to afford crude residue. It was purified by prep HPLC to afford 3-chlorobenzyl ((2S)-3-cyclohexyl-1-(((2S)-1-(diethoxyphosphoryl)-1-hydroxy-5-oxo-5-(1,2,4,5-tetrahydro-3H-benzo[d]azepin-3-yl)pentan-2-yl)amino)-1-oxopropan-2-yl)carbamate (Compound A60). TLC system: 80% Ethyl acetate in pet ether Rf: 0.3 LCMS (ESI): m/z 720.62 (M+H)⁺

### Synthesis of Compounds A65 and A115

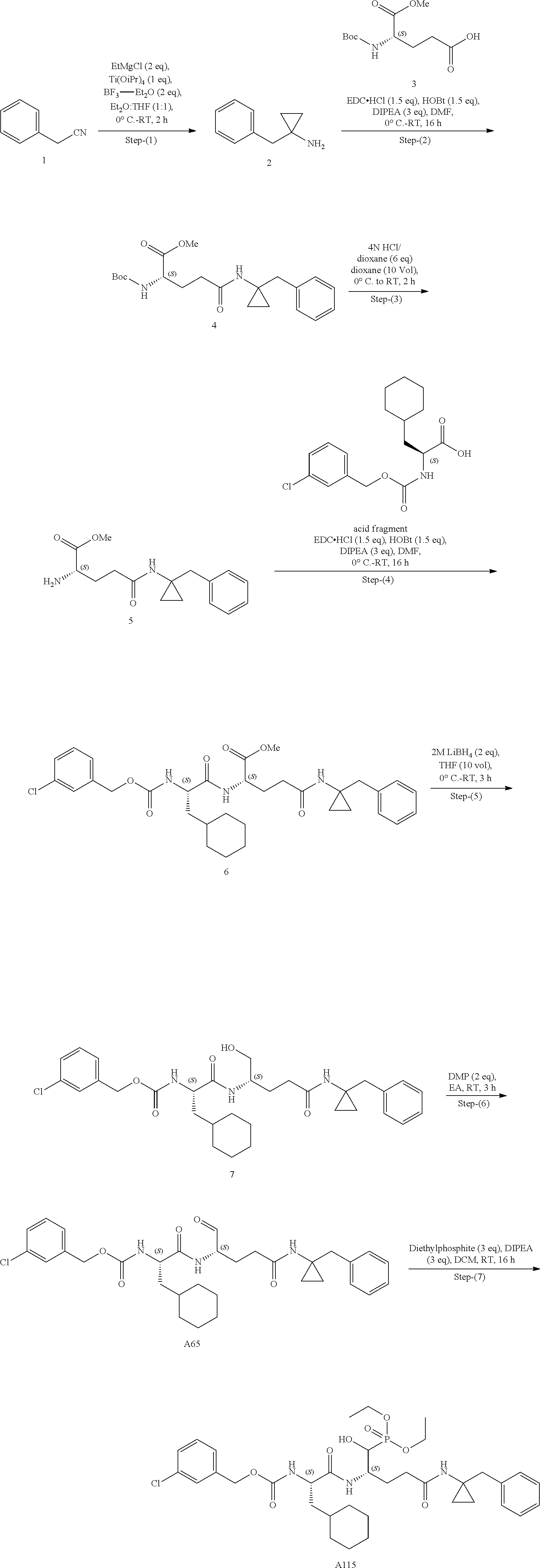

### 1-benzylcyclopropan-1-amine (2)

To a stirred solution of 2-phenylacetonitrile (1) (2 g, 17.094 mmol) in Et₂O:THF (1:1) (20 mL) were added Titanium isopropoxide (5.14 g, 18.119 mmol) and followed by added 2M Ethyl magnesium chloride in THE (17 mL, 34.188 mmol) slowly drop wise for 10 min at 0° C. Then the reaction mixture stirred at RT for 1 h and then added BF₃-Et₂O (4.8 mL, 34.188 mmol) slowly at 0° C. for 15 min (exothermic occurred) and stirred at room temperature for 1 h. The progress of the reaction was monitored by TLC and LCMS. After 1 h, the reaction mixture was poured in 10% NaOH solution (100 mL) white precipitate formed. The reaction mixture filtered through celite bed and washed with ethyl acetate (2×50 mL) and filtrate washed with brine solution (100 ml) and combined organic layer was dried over sodium sulfate, filtered and evaporated under reduced pressure. The crude residue was purified by combi-flash compound eluted at 70% Ethyl acetate in pet ether to afford 1-benzylcyclopropan-1-amine (2). TLC system: 80% Ethyl acetate in Pet ether Rf: 0.3 LCMS (ESI): m/z 148.11 [M+H]⁺

### Methyl N5-(1-benzylcyclopropyl)-N2-(tert-butoxycarbonyl)-L-glutaminate (4)

To a stirred solution of (S)-4-((tert-butoxycarbonyl)amino)-5-methoxy-5-oxopentanoic acid (3) (1 g, 1.915 mmol) in DMF (10 mL) were added EDC.HCl (731 mg, 3.831 mmol), HOBT (517 mg, 3.831 mmol), DIPEA (1 mL, 5.747 mmol) and 1-benzylcyclopropan-1-amine (2) (337 mg, 2.298 mmol) at 0° C. simultaneously and stirred at room temperature for 16 h. The progress of the reaction was monitored by TLC and LCMS. After 16 h, the reaction mixture was diluted with ice water (50 mL), extracted with ethyl acetate (2×50 mL). The combined organic layer was dried over sodium sulfate, filtered and evaporated under reduced pressure. The crude residue was purified by combi-flash compound eluted at 40% Ethyl acetate in pet ether to afford methyl N5-(1-benzylcyclopropyl)-N2-(tert-butoxycarbonyl)-L-glutaminate (4). TLC system: 50% Ethyl acetate in Pet ether Rf: 0.5 LCMS (ESI): m/z=413 [M+Na]⁺

### Methyl N5-(1-benzylcyclopropyl)-L-glutaminate hydrochloride (5)

To a stirred solution of methyl N5-(1-benzylcyclopropyl)-N2-(tert-butoxycarbonyl)-L-glutaminate (4) (1.0 g, 2.564 mmol) in 1,4-dioxane (10 mL) was added 4N HCl in dioxane (20 mL) with drop wise at 0° C. and the reaction mixture was stirred at RT for 2 h. The progress of the reaction was monitored by TLC and LCMS. After consumption of starting material, the reaction mixture was evaporated under reduced pressure to obtained crude compound, the resulting crude triturated with diethyl ether to afford methyl N5-(1-benzylcyclopropyl)-L-glutaminate hydrochloride (5).

TLC system: 10% Methanol in DCM Rf: 0.2 LCMS (ESI): m/z 291.28 [M+H]⁺

### Methyl N5-(1-benzylcyclopropyl)-N2-((S)-2-((((3-chlorobenzyl)oxy)carbonyl)amino)-3-cyclohexylpropanoyl)-L-glutaminate (6)

To a stirred solution of (S)-2-((((3-chlorobenzyl)oxy)carbonyl)amino)-3-cyclohexylpropanoic acid (acid fragment) (1.0 g, 2.949 mmol) DMF (10 mL) added EDC.HCl (1.12 g, 5.899 mmol), HOBT (796 mg, 5.899 mmol), DIPEA (1.6 mL, 8.849 mmol) and methyl N5-(1-benzylcyclopropyl)-L-glutaminate hydrochloride (5) (855 mg, 3.539 mmol) at 0° C. simultaneously and stirred at room temperature for 16 h. The progress of the reaction was monitored by TLC and LCMS. After 16 h, reaction mixture was diluted with ice water (30 mL), extracted with ethyl acetate (2×50 mL). The combined organic layer was dried over sodium sulfate, filtered and evaporated under reduced pressure. The crude residue was purified by combi-flash, compound eluted at 60% Ethyl acetate in pet ether to afford methyl N5-(1-benzylcyclopropyl)-N2-((S)-2-((((3-chlorobenzyl)oxy)carbonyl)amino)-3-cyclohexylpropanoyl)-L-glutaminate (6). TLC system: 80% Ethyl acetate in Pet ether Rf: 0.5 LCMS (ESI): m/z 612.88 [M+H]⁺

### 3-Chlorobenzyl ((S)-1-(((S)-5-((1-benzylcyclopropyl)amino)-1-hydroxy-5-oxopentan-2-yl)amino)-3-cyclohexyl-1-oxopropan-2-yl)carbamate (7)

To a stirred solution of methyl N5-(1-benzylcyclopropyl)-N2-((S)-2-((((3-chlorobenzyl)oxy)carbonyl)amino)-3-cyclohexylpropanoyl)-L-glutaminate (6) (700 mg, 1.143 mmol) in THE (10 mL) was added 2M LiBH₄ in THE (1.14 mL, 2.287 mmol) at 0° C. and the reaction mixture stirred for 2 h at 0° C. The progress of the reaction was monitored by TLC and LCMS. Then reaction mixture was quenched with water (10 mL) and extracted with ethyl acetate (2×30 mL). Organic layer was washed with brine solution (10 mL), dried over Na₂SO₄ and concentrated to get crude compound. It was purified combi-flash, compound eluted at 80% Ethyl acetate in pet ether to afford 3-chlorobenzyl ((S)-1-(((S)-5-((1-benzylcyclopropyl)amino)-1-hydroxy-5-oxopentan-2-yl)amino)-3-cyclohexyl-1-oxopropan-2-yl)carbamate (7) TLC system: 5% Methanol in DCM Rf: 0.2 LCMS (ESI): m/z 584.53 (M+H)⁺

### 3-Chlorobenzyl ((S)-1-(((S)-5-((1-benzylcyclopropyl)amino)-1,5-dioxopentan-2-yl)amino)-3-cyclohexyl-1-oxopropan-2-yl)carbamate (Compound A65)

To a stirred solution of 3-chlorobenzyl ((S)-1-(((S)-5-((1-benzylcyclopropyl)amino)-1-hydroxy-5-oxopentan-2-yl)amino)-3-cyclohexyl-1-oxopropan-2-yl)carbamate (7) (200 mg, 0.343 mmol) was dissolved in ethyl acetate (10 mL) was added Dess-Martin periodinane (290 mg, 0.686 mmol) at 0° C. and stirred at RT for 3 h. Reaction mixture was diluted with ethyl acetate (10 mL) and washed with sat. Hypo solution (3×10 mL), sat. NaHCO₃ solution (3×20 mL). Organic layer was dried over anhydrous Na₂SO₄, filtered and concentrated to afford 3-chlorobenzyl ((S)-1-(((S)-5-((1-benzylcyclopropyl) amino)-1,5-dioxopentan-2-yl)amino)-3-cyclohexyl-1-oxopropan-2-yl)carbamate (Compound A65).

TLC system: 80% Ethyl acetate in Pet ether Rf: 0.4

### 3-Chlorobenzyl ((2S)-1-(((2S)-5-((1-benzylcyclopropyl)amino)-1-(diethoxyphosphoryl)-1-hydroxy-5-oxopentan-2-yl)amino)-3-cyclohexyl-1-oxopropan-2-yl)carbamate (Compound A115)

To a stirred solution of 3-chlorobenzyl ((S)-1-(((S)-5-((1-benzylcyclopropyl)amino)-1,5-dioxopentan-2-yl)amino)-3-cyclohexyl-1-oxopropan-2-yl)carbamate (Compound A65) (200 mg crude, 0.344 mmol) in DCM (10 mL) added DIPEA (0.2 mL, 1.032 mmol) followed by added diethyl phosphite (0.14 mL, 1.032 mmol) and the reaction mixture stirred at RT for 16 h. The progress of the reaction was monitored by TLC and LCMS. After 16 h, the reaction mixture quenched with Sat. ammonium chloride (15 mL) and extracted with DCM (2×20 mL). Combined organic layer was dried over anhydrous Na₂SO₄, and evaporated to afford crude residue. It was purified by prep HPLC to afford 3-chlorobenzyl ((2S)-1-(((2S)-5-((1-benzylcyclopropyl)amino)-1-(diethoxyphosphoryl)-1-hydroxy-5-oxopentan-2-yl)amino)-3-cyclohexyl-1-oxopropan-2-yl)carbamate (Compound A115) lid. TLC system: 80% Ethyl acetate in Pet ether Rf: 0.3

LCMS (ESI): m/z 722.75 (M+H)⁺

### Synthesis of Compounds A179 and A117

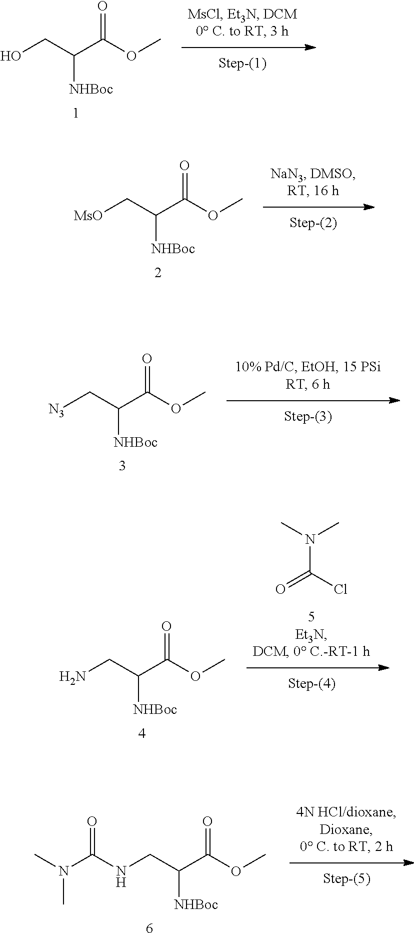

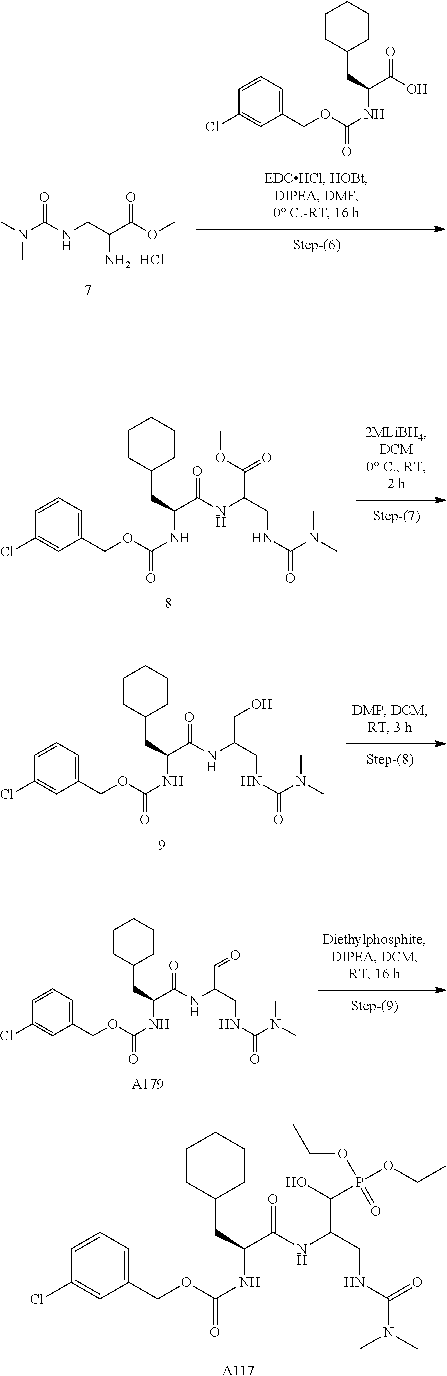

### Methyl N-(tert-butoxycarbonyl)-O-(methylsulfonyl)serinate

To a stirred solution of Methyl (tert-butoxycarbonyl)serinate (1) (24.0 g, 109.53 mmol) in DCM (240 mL) was added triethylamine (15.8 mL, 109.53 mmol) and mesyl chloride (12.48 g, 109.53 mmol) at 0° C. and reaction mixture was stirred at room temperature for 3 h. After consumption of starting material, the reaction mixture was quenched with ice water (500 mL) and extracted with Dichloromethane (2×400 mL). Combined organic layer were washed with brine solution (400 mL), dried over sodium sulfate and concentrated under reduced pressure afford to Methyl N-(tert-butoxycarbonyl)-O-(methylsulfonyl)serinate (2). TLC system: 40% Ethyl acetate in pet ether Rf: 0.5

LCMS (ESI): m/z 298.09 (M+H)⁺

### Methyl 3-azido-2-((tert-butoxycarbonyl)amino)propanoate

To a stirred solution of (Methyl N-(tert-butoxycarbonyl)-O-(methylsulfonyl)serinate (2) (15.0 g, 25.2 mmol) in DMSO (150 mL) was added sodium azide (8.2 g, 63.13 mmol) and stirred at room temperature for 16 h. After consumption of starting material, the reaction mixture was quenched with ice water (200 mL) and extracted with Diethyl ether (2×200 mL). The organic layer dried over sodium sulfate and concentrated under reduced pressure afford to crude was purified through 230-400 silica gel column chromatography by eluting with 20% ethyl acetate in pet ether to afford pure compound methyl 3-azido-2-((tert-butoxycarbonyl)amino)propanoate (3). TLC system: 20% Ethyl acetate in pet ether Rf: 0.8 LCMS (ESI): m/z 245.12 (M+H)⁺

### Methyl 3-amino-2-((tert-butoxycarbonyl)amino)propanoate

To a stirred solution of methyl 3-azido-2-((tert-butoxycarbonyl)amino)propanoate (3) (3.5 g, 14.33 mmol) in ethanol (35 mL) was treated with 10% Pd/C (660 mg), and under hydrogen atmosphere (15 psi) at RT for 6 h. After consumption of starting material, reaction mixture was filtered through celite and washed the celite bed with methanol (100 mL) and concentrated under reduced pressure. Crude was purified through 230-400 silica gel column chromatography by eluting with 10% methanol in DCM to afford pure compound afford methyl 3-amino-2-((tert-butoxycarbonyl)amino)propanoate (4). TLC system: 10% methanol in Dichloromethane Rf: 0.6 LCMS (ESI): m/z 219.30 (M+H)⁺

### Methyl-2-((tert-butoxycarbonyl)amino)-3-(3,3-diemethylureido)propanoate

To a stirred solution of methyl 3-amino-2-((tert-butoxycarbonyl)amino)propanoate (4) (0.650 g, 2.98 mmol) in DCM (65 mL) was added triethylamine (0.86 mL) and dimethylcarbamic chloride (5) (0.382 mg, 3.577 mmol) at 0° C. and stirred at room temperature for 1 h. After consumption of starting material, the reaction mixture was quenched with ice water (50 mL) and extracted with Dichloromethane (2×40 mL), dried over sodium sulfate and concentrated under reduced pressure afford to methyl-2-((tert-butoxycarbonyl)amino)-3-(3,3-diemethylureido)propanoate (6) TLC system: 10% methanol in Dichloromethane Rr: 0.8 LCMS (ESI): m/z 290.34 (M+H)⁺

### Methyl 2-amino-3-(3,3-dimethylureido)propanonate hydrochloride

To a stirred solution methyl-2-((tert-butoxycarbonyl)amino)-3-(3,3-diemethylureido)propanoate (6) (0.400 g, 16.406 mmol) in Dioxane (4 mL) was added 4N HCl/dioxane (2 mL) at 0° C. and stirred at room temperature for 2 h. After consumption of starting material (checked by TLC), reaction mixture was concentrated directly to afford methyl 2-amino-3-(3,3-dimethylureido)propanonate hydrochloride (7). TLC system: 15% methanol in Dichloromethane Rr: 0.2

LCMS (ESI): m/z 190.23 (M+H)⁺

### Methyl 2-((S)-2-((((3-chlorobenzyl)oxy)carbonyl)amino)-3-cyclohexylpropanamido)-3-(3,3-dimethylureido)propanoate

To a stirred solution of (S)-2-((((3-chlorobenzyl)oxy)carbonyl)amino)-3-cyclohexylpropanoic acid (acid fragment) (0.370 g, 1.091 mmol) in DMF (40 mL) was added EDC HCl (0.312 g, 1.637 mmol), HOBt (0.221 g, 1.637 mmol) and DIPEA (0.57 mL, 3.274 mmol) at 0° C. and stirred for 10 minutes. Then added methyl 2-amino-3-(3,3-dimethylureido)propanonate hydrochloride (7) (0.247 g, 1.309 mmol) at 0° C. and stirred for 16 h. After consumption of starting material, the reaction mixture was quenched with ice water (50 mL) and extracted with Ethyl acetate (2×40 mL). dried over sodium sulfate and concentrated under reduced pressure to afford crude compound, which was purified through 230-400 silica gel column chromatography by eluting with 10% methanol in Dichloromethane to afford pure compound afford methyl 2-((S)-2-((((3-chlorobenzyl)oxy)carbonyl)amino)-3-cyclohexylpropanamido)-3-(3,3-dimethylureido)propanoate (8). TLC system: 10% methanol in Dichloromethane Rr: 0.4 LCMS (ESI): m/z 511.45 (M+H)⁺

### 3-Chlorobenzyl ((2S)-3-cyclohexyl-1-((1-(3,3-dimethylureido)-3-hydroxypropan-2-yl)amino)-1-oxopropan-2-yl)carbamate

To a stirred solution of methyl 2-((S)-2-((((3-chlorobenzyl)oxy)carbonyl)amino)-3-cyclohexylpropanamido)-3-(3,3-dimethylureido)propanoate (8) (0.400 g, 7.843 mmol) in DCM (20 mL) was added 2M LiBH₄ in THE (0.78 mL) at 0° C. and stirred at 2 h. After consumption of starting material, the reaction mixture was quenched with sat.NH₄Cl solution (10 mL) and extracted with DCM (2×40 mL) dried over sodium sulfate and concentrated under reduced pressure to afford crude. The crude was purified through 230-400 silica gel column chromatography by eluting with 10% methanol in Dichloromethane to afford 3-chlorobenzyl ((2S)-3-cyclohexyl-1-((1-(3,3-dimethylureido)-3-hydroxypropan-2-yl)amino)-1-oxopropan-2-yl)carbamate (9). TLC system: 10% Methanol in dichloromethane Rr: 0.4 LCMS (ESI): m/z 483.45 (M+H)⁺

### 3-Chlorobenzyl ((2S)-3-cyclohexyl-1-((1-(3,3-dimethylureido)-3-oxopropan-2-yl)amino)-1-oxopropan-2-yl)carbamate

To a stirred solution of 3-chlorobenzyl ((2S)-3-cyclohexyl-1-((1-(3,3-dimethylureido)-3-hydroxypropan-2-yl)amino)-1-oxopropan-2-yl)carbamate (9) (0.170 g, 3.526 mmol) in DCM (20 mL) was added DMP (0.170 g, 3.526 mmol) at 0° C. and stirred at 3 h. After consumption of starting material, the reaction mixture was quenched with 10% sodium thiosulphate solution (20 mL) and extracted with sat NaHCO₃ solution and DCM (2×40 mL) dried over sodium sulfate and concentrated under reduced pressure to afford crude 3-chlorobenzyl ((2S)-3-cyclohexyl-1-((1-(3,3-dimethylureido)-3-oxopropan-2-yl)amino)-1-oxopropan-2-yl)carbamate (Compound A179). TLC system: 5% Methanol in dichloromethane Rr: 0.4 LCMS (ESI): m/z 483.45 (M+H)⁺

### 3-Chlorobenzyl ((2S)-3-cyclohexyl-1-((1-(diethoxyphosphoryl)-3-(3,3-dimethylureido)-1-hydroxypropan-2-yl)amino)-1-oxopropan-2-yl)carbamate (Compound A117)

To a stirred solution of 3-chlorobenzyl ((2S)-3-cyclohexyl-1-((1-(3,3-dimethylureido)-3-oxopropan-2-yl)amino)-1-oxopropan-2-yl)carbamate (Compound A179) (0.100 g, 0.2079 mmol) in DCM (10 mL) was added Diethylphosphite (0.0861 g, 0.6237 mmol) and DIPEA (0.0806 g, 0.6237 mmol) stirred at RT for 16 h. After consumption of starting material, the reaction mixture was quenched with ice water (50 mL) and extracted with DCM (2×40 mL), dried over sodium sulfate and concentrated under reduced pressure to afford the crude compound. The crude was purified by Prep.HPLC to afford 3-chlorobenzyl ((2S)-3-cyclohexyl-1-((1-(diethoxyphosphoryl)-3-(3,3-dimethylureido)-1-hydroxypropan-2-yl)amino)-1-oxopropan-2-yl)carbamate (Compound A117). LCMS (ESI): m/z 619.58 (M+H)

### Synthesis of Compounds A75 and A120

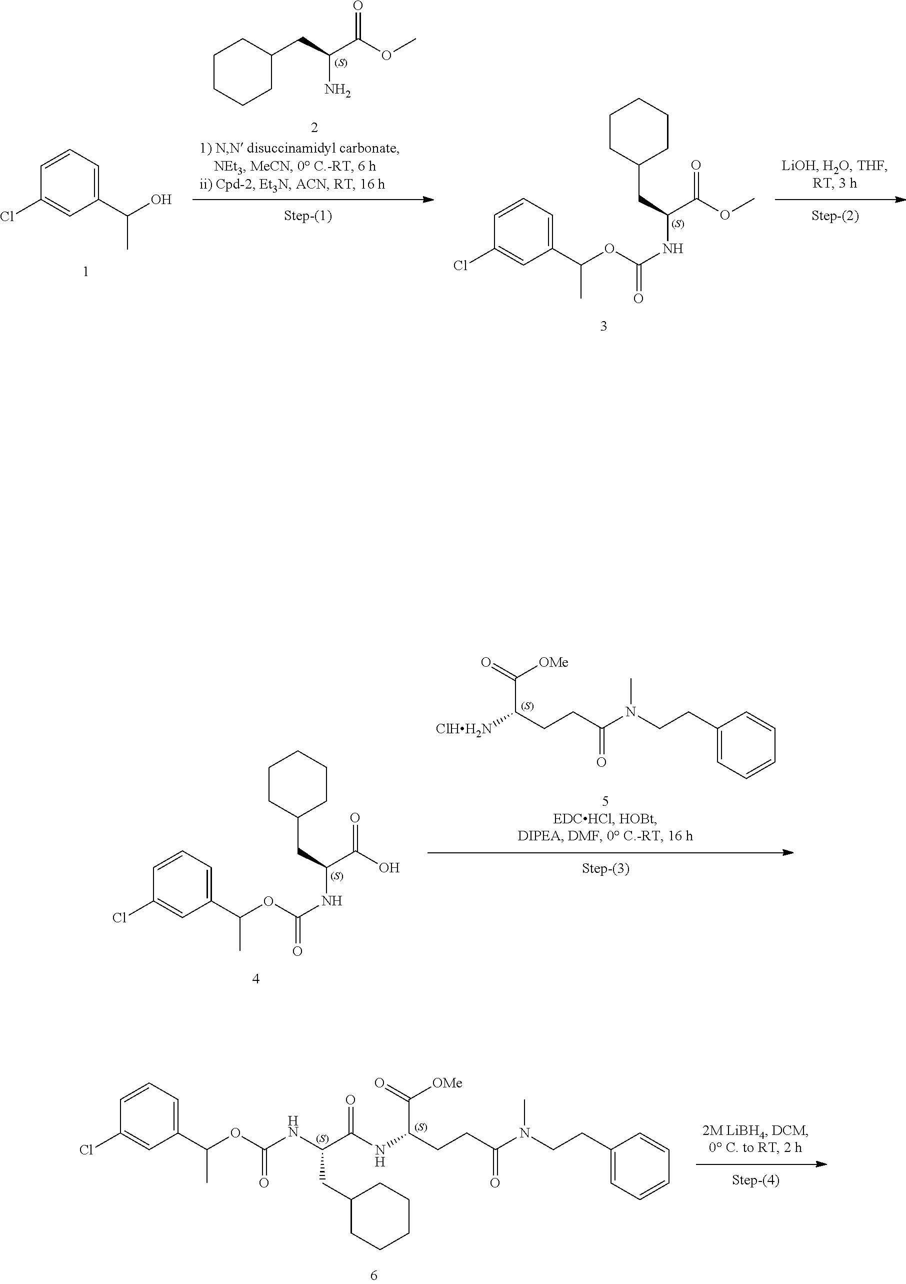

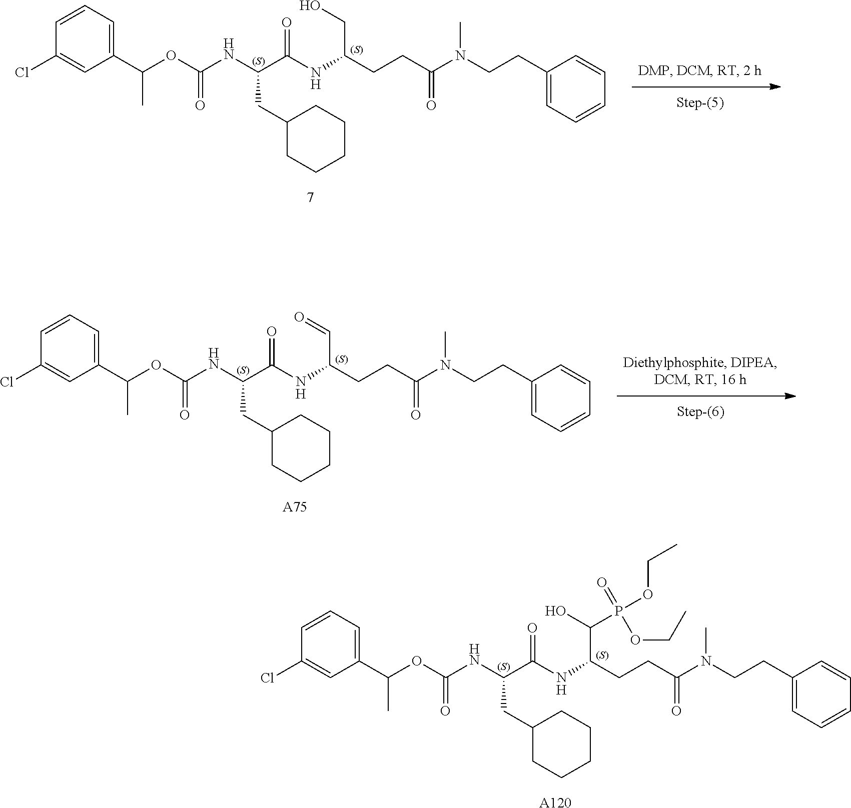

### Methyl (2S)-2-(((1-(3-chlorophenyl)ethoxy)carbonyl)amino)-3-cyclohexylpropanoate (3)

To a stirred solution of 1-(3-chlorophenyl)ethan-1-ol (1) (2 g, 12.82 mmol) in ACN (20 mL) was added N,N′disuccinamidyl carbonate (8.2 g, 32.18 mmol), followed by triethylamine (5.34 mL, 38.48 mmol) at room temperature and stirred for 3 h. The progress of the reaction was monitored by TLC. The reaction mass was used directly in the subsequent reaction.

In another RB flask, methyl (S)-2-amino-3-cyclohexylpropanoate (2) (2.96 g, 16.24 mmol) was taken in ACN (20 mL), and treated with triethylamine (5.34 mL, 38.48 mmol). The resulting reaction mixture was stirred for 5 min, then added above prepared reaction mass drop-wise and the reaction mixture stirred at room temperature for 16 h. After 16 h, the reaction mixture was quenched with ice water (100 mL) and extracted with ethyl acetate (2×100 mL), combined organic layers were washed with brine solution (50 mL), dried over sodium sulfate and evaporated under reduced pressure. The crude residue was purified by normal phase chromatography to afford methyl (2S)-2-(((1-(3-chlorophenyl)ethoxy)carbonyl)amino)-3-cyclohexylpropanoate (3). TLC system: 15% EtOAc/Pet ether Rt: 0.45

LCMS (ESI): m/z 390.32 [M+Na]+

### (2S)-2-(((1-(3-Chlorophenyl)ethoxy)carbonyl)amino)-3-cyclohexylpropanoic acid (4)

To a stirred solution of methyl (2S)-2-(((1-(3-chlorophenyl)ethoxy)carbonyl)amino)-3-cyclohexylpropanoate (3) (2 g, 5.66 mmol) in THE (20 mL) and water (10 mL), was added lithium hydroxide (407 mg, 16.90 mmol) at RT and stirred at room temperature for 3 h. The progress of the reaction was monitored by TLC and LCMS. After 3 h, the reaction mixture completely distilled under reduced pressure, crude compound acidified with aq. 1N HCl solution up to pH ˜4, and extracted with dichloromethane (2×30 mL), dried over sodium sulfate, concentrated under reduced pressure to afford crude (2S)-2-(((1-(3-Chlorophenyl)ethoxy)carbonyl)amino)-3-cyclohexylpropanoic acid (4). TLC system: 5% MeOH/DCM Rt: 0.1 LCMS (ESI): m/z 376.28 [M+Na]+

### Methyl N²-((2S)-2-(((1-(3-chlorophenyl)ethoxy)carbonyl)amino)-3-cyclohexylpropanoyl)-N⁵-methyl-N⁵-phenethyl-L-glutaminate (6)

At 0° C., to a stirred solution of (2S)-2-(((1-(3-chlorophenyl)ethoxy)carbonyl)amino)-3-cyclohexylpropanoic acid (4) (1 g, 2.834 mmol) in DMF (20 mL) was added EDC.HCl (811 mg, 4.24 mmol), HOBT (573 mg, 4.243 mmol), DIPEA (1.51 mL, 8.493 mmol) and the reaction mass was stirred for 15 min. After 15 min, added methyl N⁵-methyl-N⁵-phenethyl-L-glutaminate hydrochloride (5) (0.99 g, 4.41 mmol) and stirred at room temperature for 16 h. The progress of the reaction was monitored by TLC and LCMS. After 16 h, the reaction mixture was quenched with ice water (150 mL) and extracted with ethyl acetate (2×80 mL). Combined organic layers were washed with brine solution (80 mL), the organic layer was dried over sodium sulfate and evaporated under reduced pressure to get crude. Crude residue was purified by normal phase chromatography with eluted 60% EtOAc in Pet ether to afford methyl N²-((2S)-2-(((1-(3-chlorophenyl)ethoxy)carbonyl)amino)-3-cyclohexylpropanoyl)-N⁵-methyl-N⁵-phenethyl-L-glutaminate (6). TLC system: 5% MeOH/DCM Rf: 0.6 LCMS (ESI): m/z 614.56 [M+H]⁺

### 1-(3-Chlorophenyl)ethyl ((S)-3-cyclohexyl-1-(((S)-1-hydroxy-5-(methyl(phenethyl)amino)-5-oxopentan-2-yl)amino)-1-oxopropan-2-yl)carbamate (7)

At 0° C., to a stirred solution of methyl N²-((2S)-2-(((1-(3-chlorophenyl)ethoxy)carbonyl)amino)-3-cyclohexylpropanoyl)-N⁵-methyl-N⁵-phenethyl-L-glutaminate (6) (500 mg, 0.815 mmol) in DCM (5 mL) was added 2M LiBH₄ in THE (0.61 mL, 1.22 mmol) and the reaction mixture was stirred at RT for 2 h. The progress of the reaction was monitored by TLC and LCMS. The reaction mixture was quenched with water (20 mL) and extracted with DCM (2×20 mL). The combined organic layer was washed with brine solution (20 mL) and the organic layer was dried over Na₂SO₄ and concentrated to get crude compound. Crude compound was purified by normal phase chromatography to afford 1-(3-chlorophenyl)ethyl ((S)-3-cyclohexyl-1-(((S)-1-hydroxy-5-(methyl(phenethyl)amino)-5-oxopentan-2-yl)amino)-1-oxopropan-2-yl)carbamate (7). TLC system: 5% MeOH/DCM Rr: 0.45 LCMS (ESI): m/z 586.64 (M+H)⁺

### 1-(3-Chlorophenyl)ethyl ((S)-3-cyclohexyl-1-(((S)-5-(methyl(phenethyl)amino)-1,5-dioxopentan-2-yl)amino)-1-oxopropan-2-yl)carbamate (Compound A75)

At 0° C., to a stirred solution of 1-(3-chlorophenyl)ethyl ((S)-3-cyclohexyl-1-(((S)-1-hydroxy-5-(methyl(phenethyl)amino)-5-oxopentan-2-yl)amino)-1-oxopropan-2-yl)carbamate (7) (200 mg, 0.341 mmol) in DCM (4 mL) was added Dess-Martin periodinane (289 mg, 0.681 mmol) and stirred at RT for 3 h. The progress of the reaction was monitored by TLC and LCMS. After 3 h, the reaction mixture was diluted with ethyl acetate (15 mL) and washed with sat. NaHCO₃ solution (3×20 mL). The organic layer was washed with brine solution (20 mL) and dried over Na₂SO₄ and concentrated to get crude 1-(3-chlorophenyl)ethyl ((S)-3-cyclohexyl-1-(((S)-5-(methyl(phenethyl)amino)-1,5-dioxopentan-2-yl)amino)-1-oxopropan-2-yl)carbamate (Compound A75). TLC system: 80% EtOAc/Pet ether Rf: 0.4 LCMS (ESI): m/z 584.42 (M+H)⁺

### 1-(3-chlorophenyl)ethyl ((2S)-3-cyclohexyl-1-(((2S)-1-(diethoxyphosphoryl)-1-hydroxy-5-(methyl(phenethyl)amino)-5-oxopentan-2-yl)amino)-1-oxopropan-2-yl)carbamate (Compound A120)

To a stirred solution of 1-(3-chlorophenyl)ethyl ((S)-3-cyclohexyl-1-(((S)-5-(methyl(phenethyl)amino)-1,5-dioxopentan-2-yl)amino)-1-oxopropan-2-yl)carbamate (Compound A75) (190 mg, 0.33 mmol) was dissolved in DCM (2 mL) was added DIPEA (0.19 mL, 1.132 mmol) and diethyl phosphite (0.156 mL, 1.132 mmol) at 0° C. and stirred at RT for 16 h. Reaction mixture was quenched with ice water (10 mL) extracted with DCM (3×20 mL). Organic layer was dried over anhydrous Na₂SO₄, filtered and purified by prep HPLC to afford pure 1-(3-chlorophenyl)ethyl ((2S)-3-cyclohexyl-1-(((2S)-1-(diethoxyphosphoryl)-1-hydroxy-5-(methyl(phenyl)amino)-5-oxopentan-2-yl)amino)-1-oxopropan-2-yl)carbamate (Compound A120). TLC system: 80% EtOAc in pet ether Rf: 0.3 LCMS (ESI): m/z 722.50 (M+H)⁺

### Synthesis of Compounds A61, A121, and A122

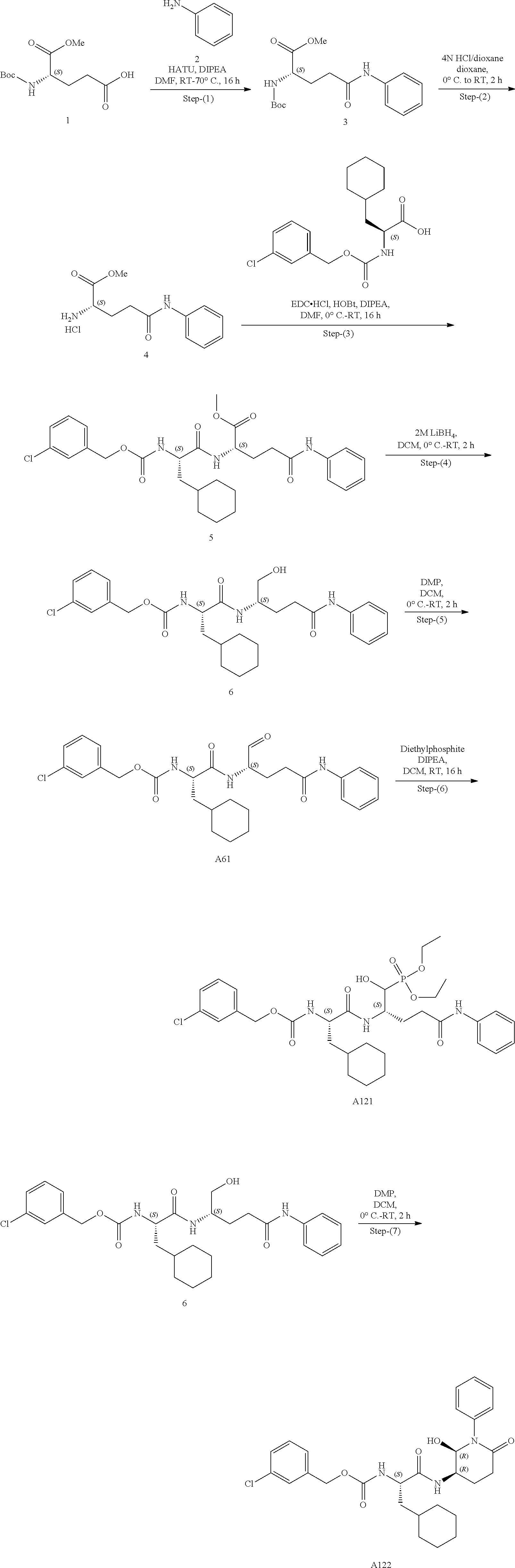

### Methyl N2-(tert-butoxycarbonyl)-N5-phenyl-L-glutaminate(3)

At 0° C., to a stirred solution of (S)-4-((tert-butoxycarbonyl)amino)-5-methoxy-5-oxopentanoic acid (1) (2 g, 7.662 mmol) in DMF (40 mL) was added HATU (1.8 g, 11.494 mmol), DIPEA (4.6 mL, 26.463 mmol) and the reaction mass was stirred for 15 min. After 15 min, added Aniline (2) (1.06 mL, 11.494 mmol) at 0° C. and stirred at room temperature for 16 h. The progress of the reaction was monitored by TLC and LCMS. After 16 h, the reaction mixture was quenched with ice water (100 mL) and extracted with ethyl acetate (2×100 mL), combined organic layers were washed with brine solution (50 mL), dried over sodium sulfate and evaporated under reduced pressure. The crude residue was purified by normal phase chromatography with eluted 100% ethyl acetate to afford methyl N2-(tert-butoxycarbonyl)-N5-phenyl-L-glutaminate (3). TLC system: 5% MeOH in DCM Rf: 0.6 LCMS (ESI): m/z 359.34 [M+Na]+

### Methyl N5-phenyl-L-glutaminate hydrochloride

To a stirred solution of methyl N2-(tert-butoxycarbonyl)-N5-phenyl-L-glutaminate (3) (2.5 g, 7.142 mmol) in 1,4-dioxane (30 mL) was added 4N HCl in dioxane (15 mL) drop wise at 0° C. and the reaction mixture was stirred at RT for 2 h. The progress of the reaction was monitored by TLC and LCMS. After 2 h, the reaction mixture was evaporated to dryness under reduced pressure to obtain crude compound, the resulting crude triturated with diethyl ether to afford methyl N5-phenyl-L-glutaminate hydrochloride (4). TLC system: 10% MeOH in DCM Rf: 0.2 LCMS (ESI): m/z 237.23 (M+H)⁺

### Methyl N2-((S)-2-((((3-chlorobenzyl)oxy)carbonyl)amino)-3-cyclohexylpropanoyl)-N5-phenyl-L-glutaminate

At 0° C., to a stirred solution of (S)-2-((((3-chlorobenzyl)oxy)carbonyl)amino)-3-cyclohexylpropanoic acid (acid fragment) (1.15 g, 3.392 mmol) (synthesis reported in Compd-17 final report) in DMF (20 mL) was added EDC.HCl (945 mg, 5.08 mmol), HOBT (680 mg, 5.08 mmol), DIPEA (1.72 mL, 10.17 mmol) and the reaction mass was stirred for 15 min. After 15 min, added methyl N5-phenyl-L-glutaminate hydrochloride (4) (0.99 g, 4.41 mmol) and stirred at room temperature for 16 h. The progress of the reaction was monitored by TLC and LCMS. After 16 h, the reaction mixture was quenched with ice water (150 mL) and extracted with ethyl acetate (2×80 mL). Combined organic layers were washed with brine solution (80 mL), the organic layer was dried over sodium sulfate and evaporated under reduced pressure to get crude. Crude residue was purified by normal phase chromatography with eluted 60% EtOAc in Pet ether to afford methyl N2-((S)-2-((((3-chlorobenzyl)oxy)carbonyl)amino)-3-cyclohexylpropanoyl)-N5-phenyl-L-glutaminate (5). TLC system: 70% EtOAc/Pet ether Rf: 0.4 LCMS (ESI): m/z 558.85 (M+H)⁺

### 3-Chlorobenzyl ((S)-3-cyclohexyl-1-(((S)-1-hydroxy-5-oxo-5-(phenylamino)pentan-2-yl)amino)-1-oxopropan-2-yl)carbamate (6)

At 0° C., to a stirred solution of methyl N2-((S)-2-((((3-chlorobenzyl)oxy)carbonyl)amino)-3-cyclohexylpropanoyl)-N5-phenyl-L-glutaminate (5) (800 mg, 1.34 mmol) in DCM (8 mL) was added 2M LiBH₄ in THE (2.01 mL, 4.02 mmol) and the reaction mixture was stirred at RT for 2 h. The progress of the reaction was monitored by TLC and LCMS. The reaction mixture was quenched with water (20 mL) and extracted with EtOAc (2×20 mL). The combined organic layer was washed with brine solution (20 mL) and the organic layer was dried over Na₂SO₄ and concentrated to get crude compound. Crude compound was purified by normal phase chromatography to afford 3-chlorobenzyl ((S)-3-cyclohexyl-1-(((S)-1-hydroxy-5-oxo-5-(phenylamino)pentan-2-yl)amino)-1-oxopropan-2-yl)carbamate (6). TLC system: 80% EtOAc/Pet ether Rf: 0.3 LCMS (ESI): m/z 530.43 (M+H)⁺

### 3-Chlorobenzyl ((S)-3-cyclohexyl-1-(((S)-1,5-dioxo-5-(phenylamino)pentan-2-yl)amino)-1-oxopropan-2-yl)carbamate (Compound A61)

At 0° C., to a stirred solution of 3-chlorobenzyl ((S)-3-cyclohexyl-1-(((S)-1-hydroxy-5-oxo-5-(phenylamino)pentan-2-yl)amino)-1-oxopropan-2-yl)carbamate (6) (200 mg, 0.378 mmol) in ethyl acetate (5 mL) was added Dess-Martin periodinane (480 mg, 1.134 mmol) and stirred at RT for 3 h. The progress of the reaction was monitored by TLC and LCMS. After 3 h, the reaction mixture was diluted with ethyl acetate (15 mL) and washed with sat. NaHCO₃ solution (3×20 mL). The organic layer was washed with brine solution (20 mL) and dried over Na₂SO₄ and concentrated to get crude compound. Crude was purified by prep HPLC to afford 3-chlorobenzyl ((S)-3-cyclohexyl-1-(((S)-1,5-dioxo-5-(phenylamino)pentan-2-yl)amino)-1-oxopropan-2-yl)carbamate (Compound A61). TLC system: 80% EtOAc/Pet ether Rf: 0.4 LCMS (ESI): m/z 528.42 (M+H)⁺

### 3-Chlorobenzyl ((2S)-3-cyclohexyl-1-(((2S)-1-(diethoxyphosphoryl)-1-hydroxy-5-oxo-5-(phenylamino)pentan-2-yl)amino)-1-oxopropan-2-yl)carbamate (Compound A121)

To a stirred solution of 3-chlorobenzyl ((S)-3-cyclohexyl-1-(((S)-1,5-dioxo-5-(phenylamino)pentan-2-yl)amino)-1-oxopropan-2-yl)carbamate (Compound A61) (199 mg, 0.377 mmol) was dissolved in DCM (2 mL) was added DIPEA (0.19 mL, 1.132 mmol) and diethyl phosphite (0.156 ml, 1.132 mmol) at 0° C. and stirred at RT for 16 h. Reaction mixture was quenched with ice water (10 mL) extracted with DCM (3×20 mL). Organic layer was dried over anhydrous Na₂SO₄, filtered and purified by prep HPLC to afforded pure 3-chlorobenzyl ((2S)-3-cyclohexyl-1-(((2S)-1-(diethoxyphosphoryl)-1-hydroxy-5-oxo-5-(phenylamino)pentan-2-yl)amino)-1-oxopropan-2-yl)carbamate (Compound A121). TLC system: 80% EtOAc in pet ether Rf: 0.3 LCMS (ESI): m/z 666.87 (M+H)⁺

### 3-Chlorobenzyl ((S)-3-cyclohexyl-1-(((2R,3R)-2-hydroxy-6-oxo-1-phenylpiperidin-3-yl)amino)-1-oxopropan-2-yl)carbamate (Compound A122)

At 0° C., to a stirred solution of 3-chlorobenzyl ((S)-3-cyclohexyl-1-(((S)-1-hydroxy-5-oxo-5-(phenylamino)pentan-2-yl)amino)-1-oxopropan-2-yl)carbamate (6) (250 mg, 0.472 mmol) in ethyl acetate (5 mL) was added Dess-Martin periodinane (601 mg, 1.417 mmol) and stirred at RT for 3 h. The progress of the reaction was monitored by TLC and LCMS. After 3 h, the reaction mixture was diluted with ethyl acetate (15 mL) and washed with sat. NaHCO₃ solution (3×20 mL). The organic layer was washed with brine solution (20 mL) and dried over Na₂SO₄ and concentrated to get crude compound. Crude was purified by prep HPLC to afford 3-chlorobenzyl ((S)-3-cyclohexyl-1-(((2S,3R)-2-hydroxy-6-oxo-1-phenylpiperidin-3-yl)amino)-1-oxopropan-2-yl)carbamate (Compound A122). TLC system: 70% EtOAc/Pet ether Rf: 0.4 LCMS (ESI): m/z 582.52 (M+H)⁺

### Synthesis of Compounds A167, A123, and A171

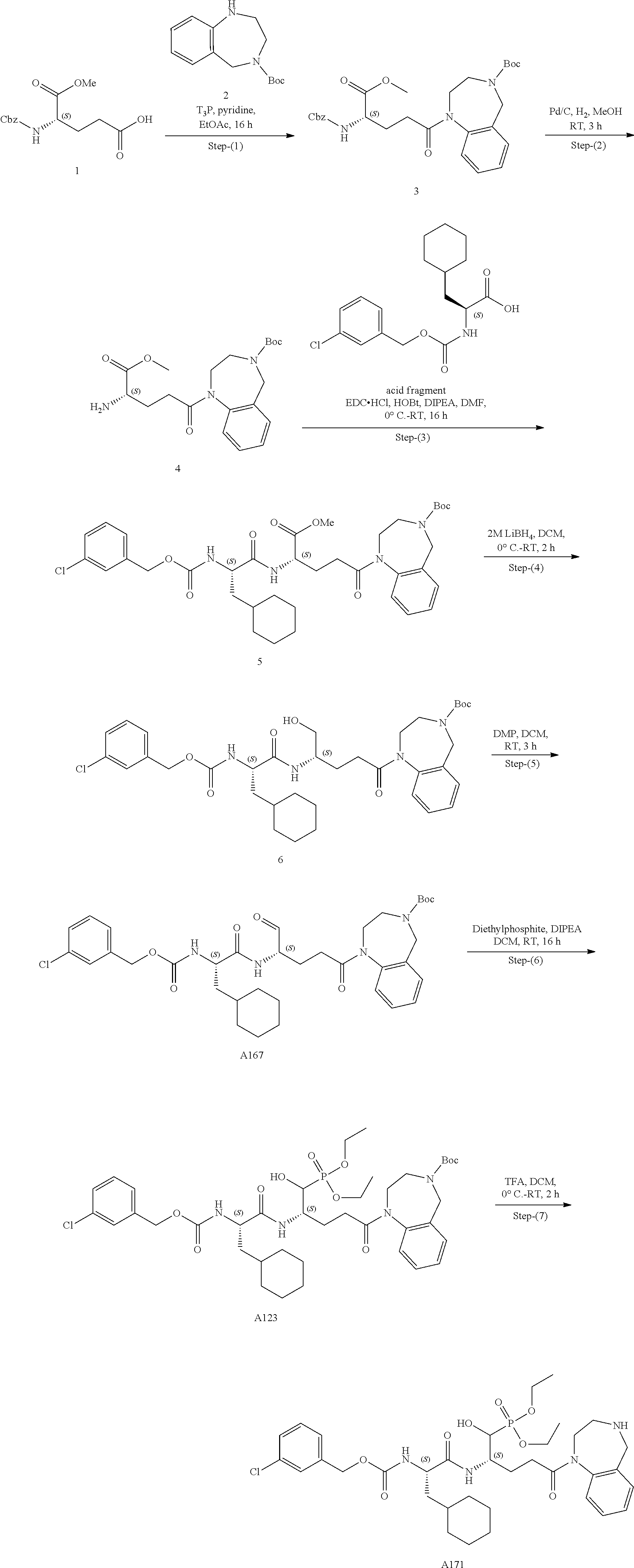

### tert-Butyl (S)-1-(4-(((benzyloxy)carbonyl)amino)-5-methoxy-5-oxopentanoyl)-1,2,3,5-tetrahydro-4H-benzo[e][1,4]diazepine-4-carboxylate (3)

A mixture of acid (1) (2 g, 8.064 mmol), amine (2) (2.85 g, 9.6774 mmol), and pyridine (2 mL, 1 vol) in 40 mL of EtOAc at 0° C. was treated with T₃P (4.74 mL, 50 wt % in EtOAc, 16.129 mmol). The resulting mixture was stirred at RT for 16 h. The progress of the reaction was monitored by TLC and LCMS. After 16 h, reaction mixture was quenched with 1N HCl (20 mL) and added water (50 mL), extracted with ethyl acetate (2×50 mL), the combined organic layer was dried over sodium sulfate, filtered and evaporated under reduced pressure. The crude residue was purified by silica gel column by eluting with 50% ethyl acetate in pet ether to afford tert-butyl (S)-1-(4-(((benzyloxy)carbonyl)amino)-5-methoxy-5-oxopentanoyl)-1,2,3,5-tetrahydro-4H-benzo[e][1,4]diazepine-4-carboxylate (3). TLC system: 5% MeOH in DCM R_{f}: 0.55 LCMS (ESI): m/z 548.41 (M+Na)+

### tert-Butyl (S)-1-(4-amino-5-methoxy-5-oxopentanoyl)-1,2,3,5-tetrahydro-4H-benzo[e][1,4]diazepine-4-carboxylate (4)

To a stirred solution of tert-butyl (S)-1-(4-(((benzyloxy)carbonyl)amino)-5-methoxy-5-oxopentanoyl)-1,2,3,5-tetrahydro-4H-benzo[e][1,4]diazepine-4-carboxylate (3) in MeOH (40 mL) was added 10% Pd/C (1.8 g, 50% by wet) at RT and the reaction mixture was stirred at RT for 3 h. The progress of the reaction was monitored by TLC and LCMS. After 3 h, reaction mixture was filtered through celite and evaporated under reduced pressure to get the crude tert-butyl (S)-1-(4-amino-5-methoxy-5-oxopentanoyl)-1, 2,3,5-tetrahydro-4H-benzo[e][1,4]diazepine-4-carboxylate (4). TLC system: 5% Methanol in DCM Rr: 0.2 LCMS (ESI): m/z 392.49 (M+H)⁺

### tert-Butyl 1-((S)-4-((S)-2-((((3-chlorobenzyl)oxy)carbonyl)amino)-3-cyclohexylpropanamido)-5-methoxy-5-oxopentanoyl)-1,2,3,5-tetrahydro-4H-benzo[e][1,4]diazepine-4-carboxylate (5)

At 0° C., to a stirred solution of (S)-2-((((3-chlorobenzyl)oxy)carbonyl)amino)-3-cyclohexylpropanoic acid (1 g, 2.949 mmol) in DMF (20 mL) was added EDC.HCl (845 mg, 4.424 mmol), HOBT (597 mg, 4.424 mmol), DIPEA (1.5 mL, 8.849 mmol) and tert-butyl (S)-1-(4-amino-5-methoxy-5-oxopentanoyl)-1,2,3,5-tetrahydro-4H-benzo[e][1,4]diazepine-4-carboxylate (4) (1.38 g, 3.539 mmol) and stirred at room temperature for 16 h. The progress of the reaction was monitored by TLC and LCMS. After 16 h, reaction mixture was quenched with ice water (30 mL), extracted with ethyl acetate (2×30 mL), the combined organic layer was dried over sodium sulfate and evaporated under reduced pressure. The crude residue was purified by silica gel column by eluting with 40% ethyl acetate in pet ether to afford tert-Butyl 1-((S)-4-((S)-2-((((3-chlorobenzyl)oxy)carbonyl)amino)-3-cyclohexylpropanamido)-5-methoxy-5-oxopentanoyl)-1,2,3,5-tetrahydro-4H-benzo[e][1,4]diazepine-4-carboxylate (5). TLC system: 5% Methanol in DCM Rr: 0.4 LCMS (ESI): m/z 713.52 (M+H)⁺

### tert-Butyl 1-((S)-4-((S)-2-((((3-chlorobenzyl)oxy)carbonyl)amino)-3-cyclohexylpropanamido)-5-hydroxypentanoyl)-1, 2,3,5-tetrahydro-4H-benzo[e][1,4]diazepine-4-carboxylate (6)

To a stirred solution of tert-Butyl 1-((S)-4-((S)-2-((((3-chlorobenzyl)oxy)carbonyl)amino)-3-cyclohexylpropanamido)-5-methoxy-5-oxopentanoyl)-1,2,3,5-tetrahydro-4H-benzo[e][1,4]diazepine-4-carboxylate (5) (600 mg, 0.8426 mmol) in DCM (10 mL) was added 2M LiBH₄ in THE (0.63 mL, 1.264 mmol) at 0° C. and the reaction mixture stirred for 2 h at RT. The progress of the reaction was monitored by TLC and LCMS. After 2 h, reaction mixture was quenched with water (20 mL) and extracted with DCM (2×30 mL). Organic layer was washed with brine solution (30 mL), and combined organic layer was dried over Na₂SO₄ and concentrated to afford the crude tert-Butyl 1-((S)-4-((S)-2-((((3-chlorobenzyl)oxy)carbonyl)amino)-3-cyclohexylpropanamido)-5-hydroxypentanoyl)-1,2,3,5-tetrahydro-4H-benzo[e][1,4]diazepine-4 carboxylate (6). TLC system: 5% MeOH in DCM Rr 0.3 LCMS (ESI): m/z 707.47 (M+Na)+

### tert-Butyl 1-((S)-4-((S)-2-((((3-chlorobenzyl)oxy)carbonyl)amino)-3-cyclohexylpropanamido)-5-oxopentanoyl)-1,2,3,5-tetrahydro-4H-benzo[e][1,4]diazepine-4-carboxylate (Compound A167)

To a stirred solution of tert-Butyl 1-((S)-4-((S)-2-((((3-chlorobenzyl)oxy)carbonyl)amino)-3-cyclohexylpropanamido)-5-hydroxypentanoyl)-1,2,3,5-tetrahydro-4H-benzo[e][1,4]diazepine-4 carboxylate (6) (250 mg, 0.3654 mmol) in DCM (5 mL) was added Dess-Martin periodinane (464 mg, 1.0964 mmol) at 0° C. and stirred at RT for 3 h. The progress of the reaction was monitored by TLC and LCMS. Reaction mixture was diluted with DCM (50 mL) and washed with sat. NaHCO₃ solution (3×20 mL) followed by sat. Hypo solution (3×20 mL). Organic layer was dried over anhydrous Na₂SO₄, filtered and concentrated to get crude compound tert-Butyl 1-((S)-4-((S)-2-((((3-chlorobenzyl)oxy)carbonyl)amino)-3-cyclohexylpropanamido)-5-oxopentanoyl)-1,2,3,5-tetrahydro-4H-benzo[e][1,4]diazepine-4-carboxylate (Compound A167) which was used directly in the next step. TLC system: 5% Methanol in DCM Rr: 0.5 LCMS (ESI): m/z 683.43 (M+H)⁺

### tert-Butyl 1-((4S)-4-((S)-2-((((3-chlorobenzyl)oxy)carbonyl)amino)-3-cyclohexylpropanamido)-5-(diethoxyphosphoryl)-5-hydroxypentanoyl)-1,2,3,5-tetrahydro-4H-benzo[e][1,4]diazepine-4-carboxylate (Compound A123)

To a stirred solution of tert-Butyl 1-((S)-4-((S)-2-((((3-chlorobenzyl)oxy)carbonyl)amino)-3-cyclohexyl propanamido)-5-oxopentanoyl)-1,2,3,5-tetrahydro-4H-benzo[e][1,4]diazepine-4-carboxylate (Compound A167) (260 mg, 0.3812 mmol) in DCM (5 mL) was added DIPEA (0.2 mL, 1.143 mmol) followed by diethylphosphite (0.14 mL, 1.143 mmol) and the reaction mixture stirred at RT for 16 h. The progress of the reaction was monitored by TLC and LCMS. After 16 h, the reaction mixture quenched with ammonium chloride (15 mL) and extracted with DCM (2×15 mL). Combined organic layer was dried over anhydrous Na₂SO₄, and evaporated to afford crude residue. It was purified prep HPLC to afford tert-Butyl 1-((4S)-4-((S)-2-((((3-chlorobenzyl)oxy)carbonyl)amino)-3-cyclohexylpropanamido)-5-(diethoxy phosphoryl)-5-hydroxypentanoyl)-1,2,3,5-tetrahydro-4H-benzo[e][1,4]diazepine-4-carboxylate (Compound A123). TLC system: 5% MeOH in DCM Rr: 0.45 LCMS (ESI): m/z 821.63 (M+H)⁺

### 3-Chlorobenzyl ((2S)-3-cyclohexyl-1-(((2S)-1-(diethoxyphosphoryl)-1-hydroxy-5-oxo-5-(2,3,4,5-tetrahydro-1H-benzo[e][1,4]diazepin-1-yl)pentan-2-yl)amino)-1-oxopropan-2-yl)carbamate (Compound A171)

To a stirred solution of tert-butyl 1-((4S)-4-((S)-2-((((3-chlorobenzyl)oxy)carbonyl)amino)-3-cyclohexylpropanamido)-5-(diethoxyphosphoryl)-5-hydroxypentanoyl)-1,2,3,5-tetrahydro-4H-benzo[e][1,4]diazepine-4-carboxylate (Compound A123) (50 mg, 0.0609 mmol) in 1,4-dioxane (2 mL) was added 4N HCl in dioxane (0.5 mL) with drop wise at 0° C. and the reaction mixture was stirred at RT for 2 h. The progress of the reaction was monitored by TLC and LCMS. After consumption of starting material, the reaction mixture was evaporated under reduced pressure to obtained crude residue. It was purified SFC to afford 3-chlorobenzyl ((2S)-3-cyclohexyl-1-(((2S)-1-(diethoxyphosphoryl)-1-hydroxy-5-oxo-5-(2,3,4,5-tetrahydro-1H-benzo[e][1,4]diazepin-1-yl)pentan-2-yl)amino)-1-oxopropan-2-yl)carbamate (Compound A171). TLC system: 10% MeOH in DCM Rr: 0.3 LCMS (ESI): m/z 721.90 (M+H)⁺

### Synthesis of Compound A134

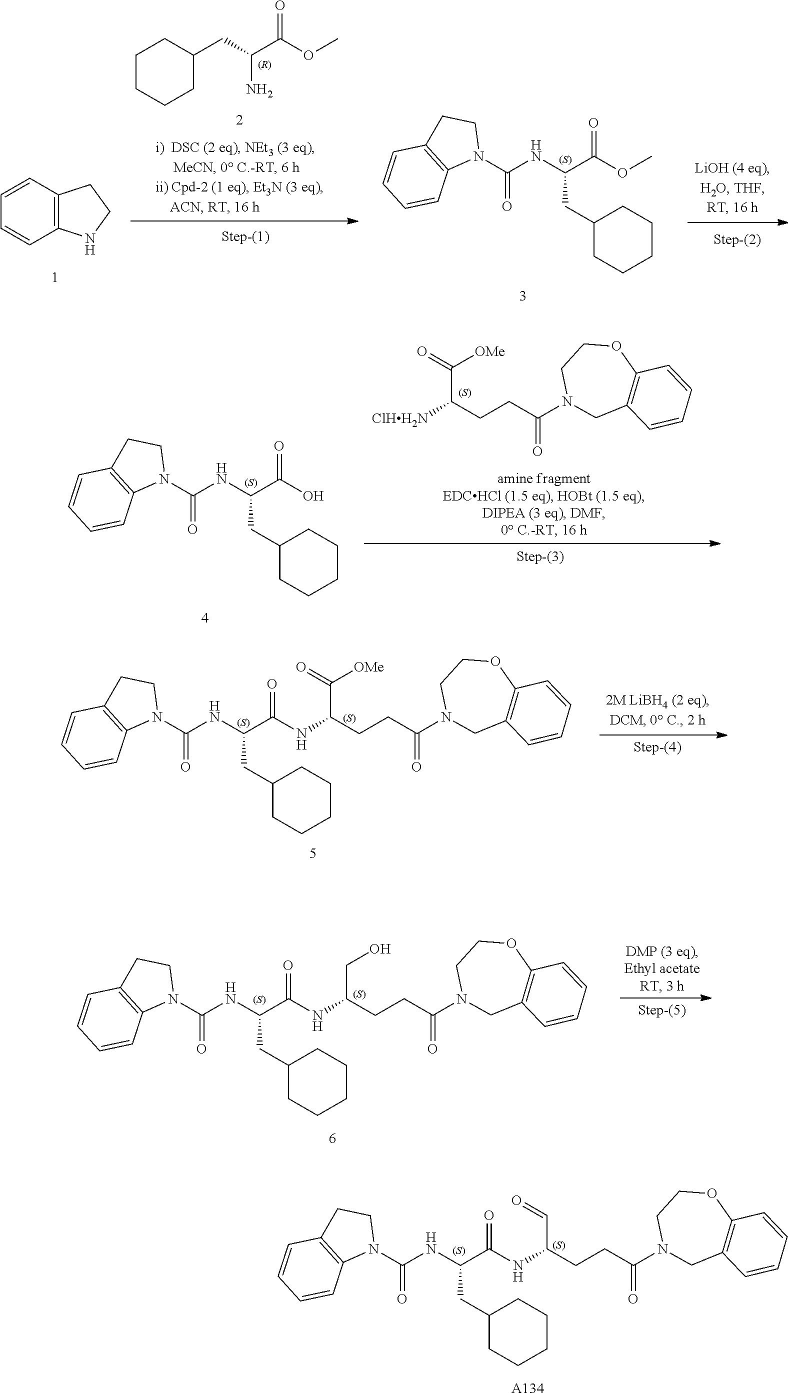

### Methyl (S)-3-cyclohexyl-2-(indoline-1-carboxamido)propanoate (3)

To a stirred solution of indoline (1) (3 g, 25.21 mmol) in ACN (30 mL) was added N,N′disuccinamidyl carbonate (12.9 g, 50.42 mmol), followed by triethylamine (4.7 mL, 0.327 mmol) at room temperature and stirred for 3 h. The progress of the reaction was monitored by TLC. The reaction mass was used directly in the subsequent reaction.

In another RB flask, methyl (S)-2-amino-3-cyclohexylpropanoate (2) (4 g, 21.73 mmol) was taken in ACN (20 mL), and treated with triethylamine (9.1 mL, 65.21 mmol). The resulting reaction mixture was stirred for 5 min, then added above prepared reaction mass drop-wise and the reaction mixture stirred at room temperature for 16 h. After 16 h, the reaction mixture was quenched with ice water (100 mL) and extracted with ethyl acetate (2×100 mL), combined organic layers were washed with brine solution (50 mL), dried over sodium sulfate and evaporated under reduced pressure. The crude residue was purified by normal phase chromatography to afford methyl (S)-3-cyclohexyl-2-(indoline-1-carboxamido)propanoate (3). TLC system: 50% EtOAc/Pet ether Rt: 0.45 LCMS (ESI): m/z 331.34 [M+H]⁺

### (S)-3-cyclohexyl-2-(indoline-1-carboxamido)propanoic acid (4)

To a stirred solution of methyl (S)-3-cyclohexyl-2-(indoline-1-carboxamido)propanoate (3) (2 g, 6.06 mmol) in THE (20 mL) and water (10 mL), was added lithium hydroxide (436 mg, 18.18 mmol) at RT and stirred at room temperature for 3 h. The progress of the reaction was monitored by TLC and LCMS. After 3 h, the reaction mixture completely distilled under reduced pressure, crude compound acidified with aq. 1N HCl solution up to pH ˜4, and extracted with dichloromethane (2×30 mL), dried over sodium sulfate, concentrated under reduced pressure to afford crude (S)-3-cyclohexyl-2-(indoline-1-carboxamido)propanoic acid (4). TLC system: 5% MeOH/DCM Rf: 0.1 LCMS (ESI): m/z 317.49 [M+H]⁺

### Methyl (S)-2-((S)-3-cyclohexyl-2-(indoline-1-carboxamido)propanamido)-5-(2,3-dihydrobenzo[f][1,4]oxazepin-4(5H)-yl)-5-oxopentanoate (5)

At 0° C., to a stirred solution of (S)-3-cyclohexyl-2-(indoline-1-carboxamido)propanoic acid (4) (600 mg, 1.89 mmol) in DMF (20 mL) was added EDC.HCl (543 mg, 2.84 mmol), HOBT (384 mg, 2.84 mmol), DIPEA (1.1 mL, 5.67 mmol) and the reaction mass was stirred for 15 min. After 15 min, added methyl N⁵-methyl-N⁵-phenethyl-L-glutaminate hydrochloride (Amine fragment) (622 mg, 1.89 mmol) and stirred at room temperature for 16 h. The progress of the reaction was monitored by TLC and LCMS. After 16 h, the reaction mixture was quenched with ice water (150 mL) and extracted with ethyl acetate (2×80 mL). Combined organic layers were washed with brine solution (80 mL), the organic layer was dried over sodium sulfate and evaporated under reduced pressure to get crude. Crude residue was purified by normal phase chromatography with eluted 60% EtOAc in Pet ether to afford methyl (S)-2-((S)-3-cyclohexyl-2-(indoline-1-carboxamido)propanamido)-5-(2,3-dihydrobenzo[f][1,4]oxazepin-4(5H)-yl)-5-oxopentanoate (5).

TLC system: 5% MeOH/DCM Rf: 0.6 LCMS (ESI): m/z 591.17 [M+H]⁺

### N—((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)indoline-1-carboxamide (6)

At 0° C., to a stirred solution of N—((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)indoline-1-carboxamide (5) (100 mg, 0.16 mmol) in DCM (5 mL) was added 2M LiBH₄ in THE (0.14 mL, 0.34 mmol) and the reaction mixture was stirred at RT for 2 h. The progress of the reaction was monitored by TLC and LCMS. The reaction mixture was quenched with water (20 mL) and extracted with DCM (2×20 mL). The combined organic layer was washed with brine solution (20 mL) and the organic layer was dried over Na₂SO₄ and concentrated to get crude compound. Crude compound was purified by normal phase chromatography to afford N—((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)indoline-1-carboxamide (6). TLC system: 5% MeOH/DCM Rr: 0.45 LCMS (ESI): m/z 563.48 (M+H)⁺

### N—((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)indoline-1-carboxamide (Compound A134)

To a stirred solution of N—((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)indoline-1-carboxamide (6) (150 mg, 0.266 mmol) in ethyl acetate (5 mL) was added Dess-Martin periodinane (334 mg, 0.8 mmol) at 0° C. and stirred at RT for 3 h. The progress of the reaction was monitored by TLC and LCMS. Reaction mixture was diluted with ethyl acetate (50 mL) and washed with sat. NaHCO₃ solution (3×20 mL) followed by sat. Hypo solution (3×20 mL). Organic layer was dried over anhydrous Na₂SO₄, filtered and concentrated to get N—((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)indoline-1-carboxamide (Compound A134). TLC system: 5% Methanol in DCM Rr: 0.5 LCMS (ESI): m/z 561.45 (M+H)⁺

### Synthesis of Compounds A74 and A119

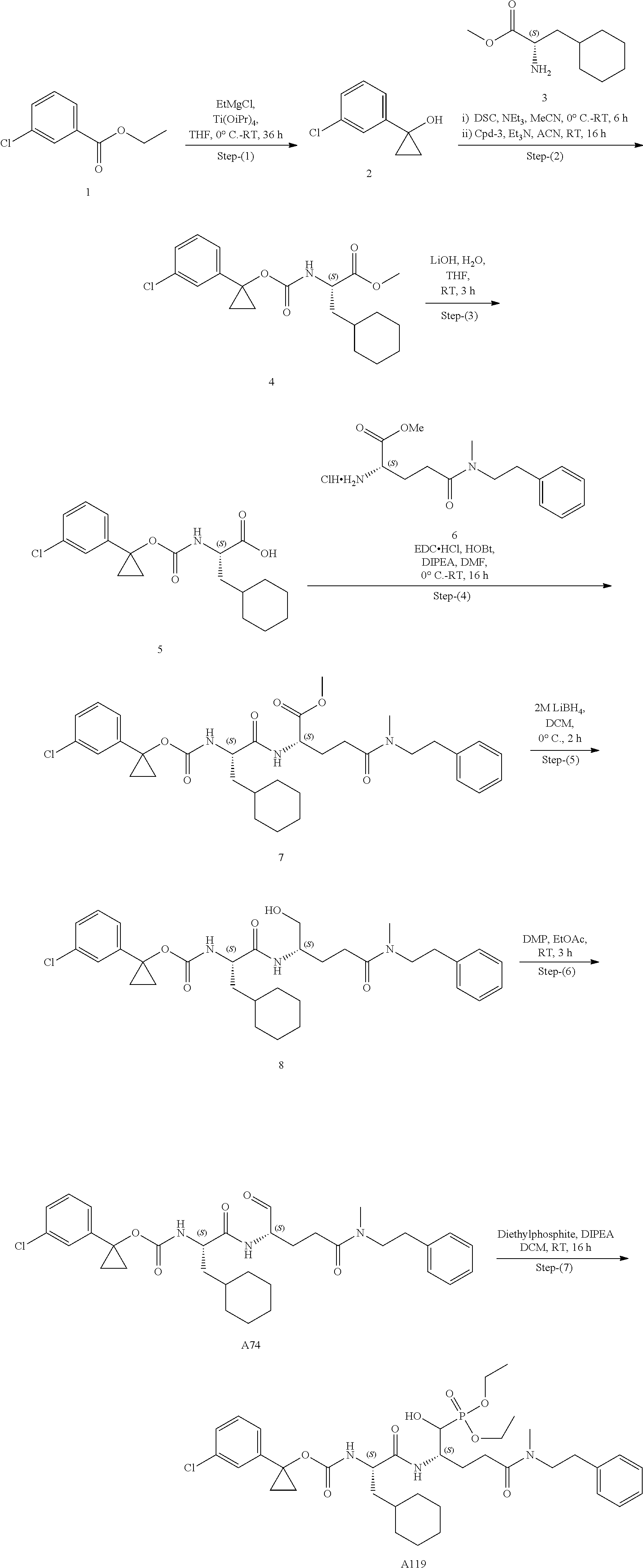

### 1-(3-Chlorophenyl) cyclopropan-1-ol (2)

To a stirred solution of 2-phenylacetonitrile (1) (5 g, 29.41 mmol) in THF (60 mL) were added Titanium isopropoxide (11.69 g, 41.17 mmol) and followed by added 2M Ethyl magnesium chloride in THF (37 mL, 73.52 mmol) slowly drop wise for 30 min at 0° C. Then the reaction mixture stirred at RT for 36 h. The progress of the reaction was monitored by TLC and LCMS. The reaction mixture was quenched with sat ammonium chloride solution (50 mL), extracted with ethyl acetate (3×40 mL), washed with brine solution (100 mL), dried over sodium sulfate, filtered and evaporated under reduced pressure. The crude residue was purified by combi-flash compound eluted at 15% Ethyl acetate in pet ether to afford 1-(3-chlorophenyl) cyclopropan-1-ol (2). TLC system: 20% Ethyl acetate in Pet ether Rf: 0.3 LCMS (ESI): m/z 151.18 [M−OH]⁺

### Methyl (S)-2-(((1-(3-chlorophenyl) cyclopropoxy) carbonyl) amino)-3-cyclohexylpropanoate (4)

To a stirred solution of 1-(3-chlorophenyl)cyclopropan-1-ol (2) (1.4 g, 8.33 mmol) in ACN (20 mL) was added N,N′disuccinamidyl carbonate (3.19 g, 12.49 mmol), followed by triethylamine (2.8 mL, 24.99 mmol) at room temperature and stirred for 6 h. The progress of the reaction was monitored by TLC. The reaction mass was used directly in the subsequent reaction.

In another RB flask, methyl (S)-2-amino-3-cyclohexylpropanoate (3) (2.70 g, 12.82 mmol) was taken in ACN (20 mL), and treated with triethylamine (3.5 mL, 24.27 mmol). The resulting reaction mixture was stirred for 5 min, then added above prepared reaction mass drop-wise and the reaction mixture stirred at room temperature for 16 h. After 16 h, the reaction mixture was quenched with ice water (100 mL) and extracted with ethyl acetate (2×50 mL), combined organic layers were washed with brine solution (50 mL), dried over sodium sulfate and evaporated under reduced pressure. The crude residue was purified by normal phase chromatography to afford methyl (S)-2-(((1-(3-chlorophenyl) cyclopropoxy) carbonyl) amino)-3-cyclohexylpropanoate (4). TLC system: 20% Ethyl acetate in Pet ether Rf: 0.6

LCMS (ESI): m/z=380.44 [M+H]⁺

### (S)-2-(((1-(3-chlorophenyl) cyclopropoxy) carbonyl) amino)-3-cyclohexylpropanoic acid (5)

To a stirred solution of methyl (S)-2-(((1-(3-chlorophenyl)cyclo propoxy) carbonyl)amino)-3-cyclohexyl propanoate (4) (1.3 g, 3.43 mmol) in THE (20 mL), water (10 mL) was added lithium hydroxide (246 mg, 10.29 mmol) at RT and stirred at room temperature for 3 h. The progress of the reaction was monitored by TLC and LCMS. After 3 h, the reaction mixture completely distilled under reduced pressure, crude compound acidified with aq. 1N HCl solution up to pH ˜3 and extracted with ethyl acetate (2×30 mL), dried over sodium sulfate, concentrated under reduced pressure to afford crude (S)-2-(((1-(3-chlorophenyl) cyclopropoxy) carbonyl) amino)-3-cyclohexylpropanoic acid (5). TLC system: 5% Methanol in DCM Rr: 0.1 LCMS (ESI): m/z 366.43 [M+H]⁺

### Methyl N2-((S)-2-(((1-(3-chlorophenyl) cyclopropoxy) carbonyl) amino)-3-cyclohexylpropanoyl)-N5-methyl-N5-phenethyl-L-glutaminate (7)

To a stirred solution of (S)-2-(((1-(3-chlorophenyl) cyclopropoxy) carbonyl)amino)-3-cyclohexylpropanoic acid (5) (1.0 g, 2.73 mmol) DMF (10 mL) added EDC.HCl (0.78 g, 4.10 mmol), HOBT (0.55 g, 4.10 mmol), DIPEA (1.5 mL, 8.21 mmol) and methyl N5-methyl-N5-phenethyl-L-glutaminate hydrochloride (6) (1.03 g, 3.28 mmol) at 0° C. simultaneously and stirred at room temperature for 16 h. The progress of the reaction was monitored by TLC and LCMS. After 16 h, reaction mixture was diluted with ice water (30 mL), extracted with ethyl acetate (2×50 mL). The combined organic layer was dried over sodium sulfate, filtered and evaporated under reduced pressure. The crude residue was purified by combi-flash, compound eluted at 60% Ethyl acetate in pet ether to afford methyl N2-((S)-2-(((1-(3-chlorophenyl)cyclopropoxy)carbonyl)amino)-3-cyclohexyl propanoyl)-N5-methyl-N5-phenethyl-L-glutaminate (7). TLC system: 80% Ethyl acetate in Pet ether Rf: 0.5 LCMS (ESI): m/z 627.47 [M+H]⁺

### 1-(3-Chlorophenyl) cyclopropyl ((S)-3-cyclohexyl-1-(((S)-1-hydroxy-5-(methyl (phenethyl) amino)-5-oxopentan-2-yl) amino)-1-oxopropan-2-yl) carbamate (8)

To a stirred solution of methyl N2-((S)-2-(((1-(3-chlorophenyl) cyclopropoxy)carbonyl)amino)-3-cyclohexylpropanoyl)-N5-methyl-N5-phenethyl-L-glutaminate (7) (1 g, 1.60 mmol) in THE (10 mL) was added 2M LiBH₄ in THE (2.4 mL, 4.80 mmol) at 0° C. and the reaction mixture stirred for 2 h at 0° C. The progress of the reaction was monitored by TLC and LCMS. Then reaction mixture was quenched with water (10 mL) and extracted with ethyl acetate (2×30 mL). Organic layer was washed with brine solution (20 mL), dried over Na₂SO₄ and concentrated to get crude compound. It was triturated with diethyl ether to afford 1-(3-chlorophenyl) cyclopropyl ((S)-3-cyclohexyl-1-(((S)-1-hydroxy-5-(methyl (phenethyl) amino)-5-oxopentan-2-yl) amino)-1-oxopropan-2-yl) carbamate (8). TLC system: 100% Ethyl acetate Rf: 0.2 LCMS (ESI): m/z 598.60 (M+H)⁺

### 1-(3-Chlorophenyl) cyclopropyl ((S)-3-cyclohexyl-1-(((S)-5-(methyl (phenethyl) amino)-1, 5-dioxopentan-2-yl) amino)-1-oxopropan-2-yl) carbamate (Compound A74)

To a stirred solution of 1-(3-chlorophenyl) cyclopropyl ((S)-3-cyclohexyl-1-(((S)-1-hydroxy-5-(methyl(phenethyl)amino)-5-oxopentan-2-yl)amino)-1-oxopropan-2-yl) carbamate (8) (150 mg, 0.25 mmol) was dissolved in ethyl acetate (10 mL) was added Dess-Martin periodinane (319 mg, 0.75 mmol) at 0° C. and stirred at RT for 3 h. Reaction mixture was diluted with ethyl acetate (10 mL) and washed with sat. Hypo solution (3×10 mL), sat. NaHCO₃ solution (3×20 mL). Organic layer was dried over anhydrous Na₂SO₄, filtered and concentrated to afford crude product, this residue was purified by normal phase chromatography by eluting 3% methanol in dichloromethane to afford 1-(3-chlorophenyl)cyclopropyl((S)-3-cyclohexyl-1-(((S)-5-methyl(phenethyl)amino)-1,5-dioxopentan-2-yl) amino)-1-oxopropan-2-yl) carbamate (Compound A74). TLC system: 5% Methanol in DCM Rf: 0.4 LCMS (ESI): m/z 596.43 (M+H)⁺

### 1-(3-Chlorophenyl) cyclopropyl ((2S)-3-cyclohexyl-1-(((2S)-1-(diethoxyphosphoryl)-1-hydroxy-5-(methyl (phenethyl) amino)-5-oxopentan-2-yl) amino)-1-oxopropan-2-yl) carbamate (Compound A119)

To a stirred solution of 1-(3-chlorophenyl) cyclopropyl ((S)-3-cyclohexyl-1-(((S)-5-methyl (phenethyl) amino)-1,5-dioxopentan-2-yl) amino)-1-oxopropan-2-yl) carbamate (Compound A74) (400 mg crude, 0.67 mmol) in DCM (10 mL) added DIPEA (0.37 mL, 2.01 mmol) followed by added diethyl phosphite (0.3 mL, 2.01 mmol) and the reaction mixture stirred at RT for 16 h. The progress of the reaction was monitored by TLC and LCMS. After 16 h, the reaction mixture quenched with Sat. ammonium chloride (15 mL) and extracted with DCM (2×20 mL). Combined organic layer was dried over anhydrous Na₂SO₄, and evaporated to afford crude residue. It was purified by prep HPLC to afford 1-(3-chlorophenyl) cyclopropyl ((2S)-3-cyclohexyl-1-(((2S)-1-(diethoxyphosphoryl)-1-hydroxy-5-(methyl (phenethyl) amino)-5-oxopentan-2-yl) amino)-1-oxopropan-2-yl) carbamate (Compound A119). TLC system: 5% Methanol in DCM Rr: 0.1 LCMS (ESI): m/z 734.78 [M+H]⁺

Other specific compounds disclosed herein were synthesized using the reaction schemes as described above, with use of the appropriate starting materials and reagents.

### Example 3: Data on Selected Compounds

### In Vitro Antiviral Assays

Norovirus Antiviral Assays:

Cell-based antiviral assays: The antiviral effects of inhibitors were examined in the Norwalk virus replicon harboring cells (HG23 cells). Briefly, confluent and semi-confluent cells were incubated with medium containing DMSO (<0.1%) or each compound (up to 100 μM) for 48 h. After the incubation, total RNA was extracted and viral genome was quantitated with real-time quantitative RT-PCR (qRT-PCR). The EC₅₀ values were determined by GraphPadPrism software. In addition to Norwalk virus replicon, the CPE (cytopathic effect) antiviral activities of the inhibitors were determined using FCoV (feline coronavirus), MERS-CoV (Middle East respiratory syndrome-related coronavirus), SARS-CoV (severe acute respiratory syndrome-related coronavirus), human coronavirus 229E, murine norovirus, and human rhinovirus.

Viral protease assays: The antiviral activities of inhibitors were determined by FRET (Fluorescence Resonance Energy Transfer) assay. Purified viral protease was incubated with the protease substrate peptide (Edans-DFHLQ/GP-Dabcyl) and inhibitor, and IC₅₀ values were subsequently determined by the fluorescence signals.

The Noro-Norwalk FRET protease assay was performed in 50 mM HEPES-Na pH 8, 50 mM NaCl, 0.4 mM EDTA, 4% glycerol, and 6 mM DTT. A self-quenching peptide substrate [5-FAM]-EPDFHLQGPEDLAKK-[TAMRA] was custom synthesized by Anaspec. Compounds were diluted in 3-fold serial dilutions to final concentrations of 200 μM to 10.2 nM. Noro protease was added to a final concentration of 1-7 μM, depending on the enzyme activity level, and the peptide substrate was added to a final concentration of 10 μM. The assay was incubated 90 minutes at 37° C. and read in a Perkin Elmer Envision with excitation at 473 nm and emission measurement at 519 nm.

The SARS2 FRET protease assay was performed in 20 mM HEPES-Na pH 7, 120 mM NaCl, 0.4 mM EDTA, 0.01% Triton, 5% glycerol, and 4 mM DTT. A self-quenching peptide substrate 5-FAM-TSA VLQ SGF RKK (5TAMRA)-NH2 was custom synthesized by Anaspec. Compounds were diluted in 3-fold serial dilutions to final concentrations of 20 μM to 1.0 nM. SARS2 protease was added to a final concentration of 25 nM, depending on the enzyme activity level, and the peptide substrate was added to a final concentration of 1.3 μM. The assay was incubated 30 minutes at 30° C. and read in a Perkin Elmer Envision with excitation at 473 nm and emission measurement at 519 nm.

Potency against Norovirus protease: IC₅₀ values are reported in Tables C and D.

TABLE C

| | | |
|---|---|---|
| | HuNV | Norwalk |
| Compound No. | GGII.4 IC₅₀ | GGI.1 IC₅₀ |

| A1 | >200 | μM | >200 | μM |
|---|---|---|---|---|
| A2 | 127.3 | μM | 169.3 | μM |
| A6 | 18.7 | μM | 2.0 | μM |
| A7 | 11.1 | μM | 1.6 | μM |
| A8 | >200 | μM | >200 | μM |
| A9 | 154.0 | μM | 14.0 | μM |
| A10 | 16.9 | μM | 3.6 | μM |
| A11 | 114.4 | μM | 133.2 | μM |
| A12 | >200 | μM | 121.6 | μM |
| A13 | >200 | μM | 166.0 | μM |
| A14 | 70.1 | μM | 50.9 | μM |
| A15 | 138.8 | μM | >200 | |
| A16 | >200 | μM | 81.9 | μM |
| A18 | 14.5 | μM | 2.7 | μM |
| A19 | 3.3 | μM | 1.0 | μM |
| A22 | >200 | μM | 142.6 | μM |
| A24 | 123.1 | μM | >200 | μM |
| A25 | 39.4 | μM | 17.2 | μM |
| A27 | 11.3 | μM | 2.7 | μM |
| A33 | >200 | μM | 117.2 | μM |
| A36 | 26.9 | μM | 5.2 | μM |
| A38 | 30.6 | μM | 10.1 | μM |
| A39 | 100.3 | μM | 118.6 | μM |
| A40 | 198.8 | μM | 116.8 | μM |
| A43 | >200 | μM | 135.3 | μM |
| A44 | 28.6 | μM | 10.7 | μM |
| A45 | 18.8 | μM | 11.0 | μM |
| A46 | 28.0 | μM | 8.6 | μM |
| A48 | 63.0 | μM | 8.7 | μM |
| A49 | 76.7 | μM | 15.0 | μM |
| A50 | 95.7 | μM | 97.4 | μM |
| A53 | 82.5 | μM | 6.7 | μM |
| A54 | 161.0 | μM | 36.3 | μM |
| A56 | 105.5 | μM | 9.3 | μM |
| A57 | 2.6 | μM | 2.3 | μM |
| A58 | 140.6 | μM | 37.1 | μM |
| A68 | 16.7 | μM | 4.6 | μM |
| A105 | 154.8 | μM | 28.2 | μM |
| A107 | 200.1 | μM | 39.6 | μM |
| A108 | 13.4 | μM | 8.0 | μM |
| A114 | 12.1 | μM | 5.2 | μM |
| A118 | 74.2 | μM | 6.6 | μM |

TABLE D

| | | |
|---|---|---|
| | HuNV | Norwalk |
| Compound No. | GGII.4 IC₅₀ | GGI.1 IC₅₀ |

| | | | | |
|---|---|---|---|---|
| B1 | 96.9 | μM | 96.9 | μM |
| B4 | 148.7 | μM | 148.7 | μM |
| B25 | 176.6 | μM | 176.6 | μM |
| B27 | 57.9 | μM | 57.9 | μM |
| B37 | >200 | μM | >200 | μM |
| B43 | 105.0 | μM | 105.0 | μM |

Results of a SARS-CoV-2 protease inhibition assay are presented in Table E below.

TABLE E

| Compound No. | IC₅₀ (μM) |
|---|---|
| A1 | >200 |
| A2 | >200 |
| A3 | >200 |
| A4 | >200 |
| A5 | >200 |
| A6 | >200 |
| A7 | 3.2 |
| A8 | 89.8 |
| A9 | >200 |
| A10 | 0.98 |
| A11 | >200 |
| A12 | 5.4 |
| A13 | >200 |
| A14 | >200 |
| A15 | >200 |
| A16 | >200 |
| A17 | 169.0 |
| A18 | >200 |
| A19 | 40.3 |
| A20 | >200 |
| A21 | >200 |
| A22 | 2.3 |
| A23 | >200 |
| A24 | >200 |
| A25 | >200 |
| A26 | 14.7 |
| A27 | 136.9 |
| A28 | >200 |
| A29 | >200 |
| A30 | >200 |
| A31 | 46.0 |
| A32 | >200 |
| A33 | >200 |
| A34 | >200 |
| A35 | >200 |
| A36 | >200 |
| A37 | >200 |
| A38 | 36.0 |
| A39 | 5.3 |
| A40 | >200 |
| A41 | 30.0 |
| A42 | >200 |
| A43 | 185.4 |
| A44 | >200 |
| A45 | >200 |
| A46 | >200 |
| A47 | >200 |
| A48 | 31.0 |
| A49 | >200 |
| A50 | 9.7 |
| A51 | >200 |
| A52 | >200 |
| A53 | 16.7 |
| A54 | 19.0 |
| A55 | >200 |
| A56 | >200 |
| A100 | >200 |
| A101 | >200 |
| A102 | >200 |
| A104 | >200 |
| A105 | >200 |
| A106 | >200 |
| A110 | >200 |
| A111 | >200 |
| A112 | >200 |
| B1 | >200 |
| B4 | 155.5 |
| B7 | >200 |
| B9 | >200 |
| B10 | 105.3 |
| B11 | >200 |
| B12 | >200 |
| B13 | >200 |
| B14 | >200 |
| B15 | >200 |
| B16 | >200 |
| B17 | >200 |
| B18 | >200 |
| B19 | >200 |
| B20 | >200 |
| B21 | 89.8 |
| B22 | >200 |
| B23 | 46.5 |
| B24 | 188.34 |
| B25 | >200 |
| B26 | >200 |
| B27 | >200 |
| B28 | >200 |
| B29 | >200 |
| B30 | >200 |
| B31 | >200 |
| B32 | >200 |
| B33 | >200 |
| B34 | >200 |
| B35 | >200 |
| B36 | >200 |
| B37 | >200 |
| B38 | 136.9 |
| B41 | >200 |
| B42 | >200 |
| B43 | >200 |
| B44 | 185.4 |
| B45 | >200 |
| B63 | 30.0 |
| B64 | >200 |
| B65 | >200 |
| B66 | >200 |

All references provided herein are incorporated herein in its entirety by reference. As used herein, all abbreviations, symbols and conventions are consistent with those used in the contemporary scientific literature. See, e.g., Janet S. Dodd, ed., The ACS Style Guide: A Manual for Authors and Editors, 2nd Ed., Washington, D.C.: American Chemical Society, 1997.

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

What is claimed is:

1. A compound having a structure of Formula (I), or a pharmaceutically acceptable salt thereof: 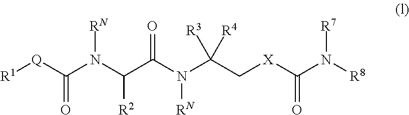
wherein
X is NR^{N}, O, or CR⁵R⁶;
Q is O, NR^{O}, or a bond;
each R^{N} is independently H or C₁₋₆alkyl;
each R^{O} is independently H or C₁₋₆alkyl;
R¹ is C₁₋₈alkyl, C₁₋₁₂alkylene-C₆₋₁₀aryl, 5- to 12-membered heterocycle having 1 to 3 ring heteroatoms selected from N, O, and S, or C₅₋₈carbocyclyl, and
the C₁₋₁₂alkylene is optionally substituted with a C₃₋₅ carbocycle,
the C₆₋₁₀aryl is optionally substituted with 1-3 halo,
the 5- to 12-membered heterocycle is optionally substituted with 1-3 substituents independently selected from COO—C₁₋₆alkyl, C₁₋₆alkylene-C₆₋₁₀aryl, and SO₂—C₁₋₆alkyl, and
the C₅₋₈carbocyclyl is optionally substituted with C₆₋₁₀aryl or C₆₋₁₀aryl substituted with 1-3 halo; or
R^{O} and R¹ together with the nitrogen to which they are attached form a 5- to 12-membered heterocycle having 1-3 ring heteroatoms selected from N, O, and S, and optionally substituted with 1-3 halo;
R² is C₁₋₆alkyl or C₁₋₆alkylene-C₅₋₈carbocyclyl, wherein C₅₋₈carbocyclyl is optionally substituted with 1-3 substituents independently selected from C₁₋₆alkyl and halo;
R³ is H or C₁₋₆alkyl;
R⁴ is C₁₋₆alkylene-OH, C₁₋₆alkylene-OH substituted with PO(OCH₂CH₂)₂ or SO₃H, CHO, C(O)-(4-8 membered heterocycle having 1-3 ring heteroatoms selected from N, O, and S, CONR^{A}R^{B}, or C(O)—C(O)NR^{N}—Y¹—X¹-A, wherein A is C₅₋₈carbocyclyl, 4-12-membered heterocycle having 1-3 ring heteroatoms selected from N, O, and S, C₆₋₁₀aryl, or 5-8-membered heteroaryl having 1-3 ring heteroatoms selected from N, O, and S, and the carbocyclyl, heterocyclyl, aryl, or heteroaryl is optionally substituted with 1-2 substituents independently selected from halo, C₁₋₆alkyl, and COO—C₁₋₆alkyl;
Y¹ is C₁₋₆alkylene optionally substituted with 1-3 substituents independently selected from halo, OH, NR^{N}R^{N}, and C₁₋₆alkoxy;
X¹ is null, NR^{N}R^{N}, C(O), SO₂, or OC(O);
R⁵ and R⁶ are each independently H or C₁₋₆alkyl;
R⁷ is C₁₋₆alkyl, C₁₋₆alkylene-O—C₁₋₆alkyl, C₁₋₆alkylene-O—C₁₋₆alkylene-O—C₁₋₆alkyl, C₁₋₆alkylene-CONH—C₁₋₆alkyl, C₀₋₆alkylene-CON(CH₃)₂, C₁₋₆alkylene-NHCONH—C₁₋₆alkyl, C₁₋₆alkylene-OCONH—C₁₋₆alkyl, C₁₋₆alkylene-NHCOO—C₁₋₆alkyl, C₁₋₆alkylene-NHSO₂—C₁₋₆alkyl, C₀₋₆alkylene-C₆₋₁₀aryl and the C₀₋₆alkylene is optionally substituted with C₃₋₅carbocyclyl, or C₁₋₆alkylene-5-8 membered heteroaryl having 1-3 ring heteroatoms selected from N, O, and S, wherein C₆₋₁₀aryl and 5-8 membered heteroaryl are optionally substituted with 1-3 substituents independently selected from C₁₋₆alkoxy and halo;
R⁸ is H or C₁₋₆alkyl; or
R⁴ and R⁸ together with the atoms to which they are attached form a 5- to 8-membered heterocycle having 1-3 ring heteroatoms selected from N, O, and S, optionally substituted with 1-3 R^{C}; or
R⁶ and R⁸ together with the atoms to which they are attached form a 5- to 8-membered heterocycle having 1-3 ring heteroatoms selected from N, O, and S, optionally substituted with CN, C₁₋₆alkylene-O—C₁₋₆alkyl, C₁₋₆alkylene-O—C₁₋₆alkylene-O—C₁₋₆alkyl, C₀₋₆alkylene-C₆₋₁₀aryl, or a 6-membered heterocycle having 1-3 ring heteroatoms selected from N, O, and S, and R⁷ can be H; or
R⁷ and R⁸ together with the nitrogen to which they are attached form a 5-12 membered heterocycle having 1-3 ring heteroatoms selected from N, O, and S, optionally substituted with halo, C₆₋₁₀aryl, or COO—C₁₋₆ alkyl;
R^{A} and R^{B} are each independently H, C₁₋₆alkyl, or C₁₋₆alkoxy; and
each R^{C} is independently OH or CONH(C₁₋₆alkyl).

2. The compound or salt of claim 1, wherein X is O.

3. The compound or salt of claim 1, wherein X is CR⁵R⁶.

4. The compound or salt of claim 3, wherein X is CH₂.

5. The compound or salt of claim 1, wherein X is NR^{N}.

6. The compound or salt of any one of claims 1 to 5, wherein each R^{N} is H.

7. The compound or salt of any one of claims 1 to 6, wherein R¹ is C₁₋₆alkyl.

8. The compound or salt of any one of claims 1 to 6, wherein R¹ is C₁₋₆alkylene-C₆₋₁₀aryl.

9. The compound or salt of claim 8, wherein C₁₋₆alkylene is substituted with a 3-5 membered carbocycle.

10. The compound or salt of claim 9 wherein C₆₋₁₀aryl of R¹ is substituted with 1-3 halo.

11. The compound or salt of claim 10, wherein R¹ is

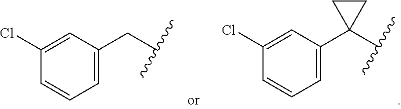 [SMILES: *Cc1cccc(Cl)c1 ; *C1(c2cccc(Cl)c2)CC1]

12. The compound or salt of claim 10, wherein R¹ is

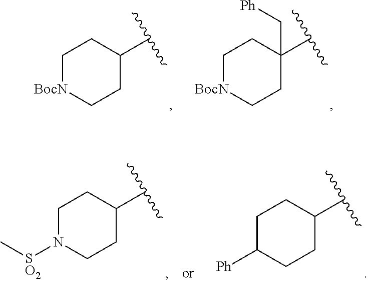 [SMILES: *C1CCN(C(=O)OC(C)(C)C)CC1 ; *C1(Cc2ccccc2)CCN(C(=O)OC(C)(C)C)CC1 ; *C1CCN(S(C)(=O)=O)CC1 ; *C1CCC(c2ccccc2)CC1]

13. The compound or salt of any of claims 1 to 12, wherein Q is a bond.

14. The compound or salt of any of claims 1 to 12, wherein Q is O.

15. The compound or salt of any of claims 1 to 12, wherein Q is NR^{O}.

16. The compound or salt of claim 15, wherein R^{O} and R¹ together with the nitrogen to which they are attached form a 5- to 12-membered heterocycle optionally substituted with 1-3 halo.

17. The compound or salt of claim 16, wherein R¹-Q- is

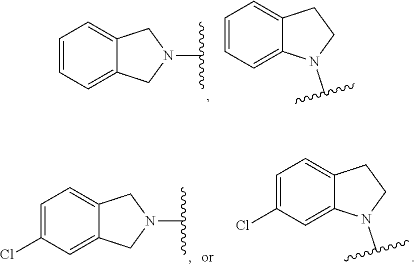

18. The compound or salt of any one of claims 1 to 17, wherein R² is C₁₋₆alkyl.

19. The compound or salt of claim 18, wherein R² is isobutyl.

20. The compound or salt of any one of claims 1 to 17, wherein R² is C₁₋₆alkylene-C₅₋₈carbocyclyl.

21. The compound or salt of claim 20, wherein C₅₋₈carbocyclyl is substituted with 1-3 substituents selected from C₁₋₆ alkyl and halo.

22. The compound or salt of claim 20, wherein R² is

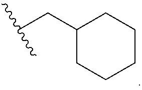 [SMILES: *CC1CCCCC1]

23. The compound or salt of any one of claims 1 to 22, wherein R³ is H.

24. The compound or salt of any one of claims 1 to 23, having a structure of Formula (IA): 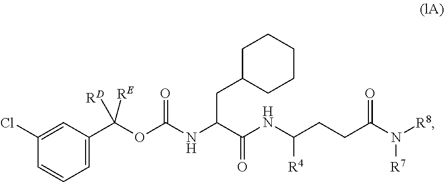
wherein R^{D} is H and R^{E} is H or C₁₋₆ alkyl or R^{D} and R^{E} together with the carbon to which they are attached form a 3-5 membered carbocycle.

25. The compound or salt of claim 24, wherein R^{D} and R^{E} are each H.

26. The compound or salt of claim 24, wherein R^{D} and R^{E} together with the carbon to which they are attached form a 3 membered carbocycle.

27. The compound or salt of any one of claims 1 to 26, wherein R⁴ is C₁₋₆alkylene-OH substituted with PO(OCH₂CH₂)₂.

28. The compound or salt of any one of claims 1 to 26, wherein R⁴ is CHO.

29. The compound or salt of any one of claims 1 to 26, wherein R⁴ is C(O)-(4-8 membered heterocycle having 1-3 ring heteroatoms selected from N, O, and S.

30. The compound or salt of any one of claims 1 to 26, wherein R⁴ is CONR^{A}R^{B}.

31. The compound or salt of any one of claims 1 to 30, wherein R⁴ is C(O)—C(O)NR^{N}—Y¹—X¹-A.

32. The compound or salt of claim 31, wherein Y¹ is C₁₋₆alkylene.

33. The compound or salt of claim 31 or 32, wherein X¹ is null.

34. The compound or salt of claim 31 or 32, wherein X¹ is NR^{N}R^{N}, C(O), SO₂, or OC(O).

35. The compound or salt of any one of claims 31 to 34, wherein A is C₅₋₈carbocyclyl or C₆₋₁₀aryl, and optionally substituted with 1-2 substituents independently selected from halo, C₁₋₆alkyl, and COO—C₁₋₆alkyl.

36. The compound or salt of any one of claims 31 to 34, wherein A is 4-12-membered heterocycle having 1-3 ring heteroatoms selected from N, O, and S, or 5-8-membered heteroaryl having 1-3 ring heteroatoms selected from N, O, and S, optionally substituted with 1-2 substituents independently selected from halo, C₁₋₆alkyl, and COO—C₁₋₆alkyl.

37. The compound or salt of claim 36, wherein A comprises pyridyl.

38. The compound or salt of any one of claims 30 to 37, wherein R^{A} is C₁₋₆alkyl.

39. The compound or salt of claim 38, wherein R^{A} is methyl.

40. The compound or salt of any one of claims 30 to 39, wherein R^{B} is C₁₋₆alkoxy.

41. The compound or salt of claim 40, wherein R^{B} is methoxy.

42. The compound or salt of any one of claims 1 to 26, wherein R⁴ and R⁸ together with the atoms to which they are attached form a 6-membered heterocycle, optionally substituted with 1-3 R^{C}.

43. The compound or salt of claim 42, wherein the 6-membered heterocycle is substituted with 1 or 2 R^{C}.

44. The compound or salt of claim 43 or 43, wherein the 6-membered heterocycle is substituted with OH.

45. The compound or salt of any one of claims 1 to 23, wherein R⁶ and R⁸ together with the atoms to which they are attached form a 5-membered heterocycle.

46. The compound or salt of claim 45, wherein the heterocycle is substituted with CN, C₁₋₆alkylene-O—C₁₋₆alkyl, C₁₋₆ alkylene-O—C₁₋₆alkylene-O—C₁₋₆alkyl, C₀₋₆alkylene-C₆₋₁₀aryl, or a 6-membered heterocycle.

47. The compound of claim 45 or 46, wherein R⁷ is H.

48. The compound or salt of any one of claims 1 to 44, wherein R⁷ is C₁₋₆alkyl.

49. The compound or salt of claim 48, wherein R⁷ is methyl.

50. The compound or salt of claim 48, wherein R⁷ is C₅alkyl.

51. The compound or salt of any one of claims 1 to 44, wherein R⁷ is C₁₋₆alkylene-O—C₁₋₆alkyl or C₁₋₆alkylene-O—C₁₋₆ alkylene-O—C₁₋₆alkyl.

52. The compound or salt of any one of claims 1 to 51, wherein R⁷ is C₁₋₆alkylene-O—C₁₋₆alkyl.

53. The compound or salt of claim 52, wherein R⁷ is C₂alkylene-O—C₂alkyl.

54. The compound or salt of any one of claims 1 to 44, wherein R⁷ is C₁₋₆alkylene-CONH—C₁₋₆alkyl, C₁₋₆alkylene-NHCONH—C₁₋₆alkyl, C₁₋₆alkylene-OCONH—C₁₋₆alkyl, C₁₋₆alkylene-NHCOO—C₁₋₆alkyl, or C₁₋₆alkylene-NHSO₂—C₁₋₆alkyl.

55. The compound or salt of any one of claims 1 to 44, wherein R⁷ is C₁₋₆alkylene-CONH—C₁₋₆alkyl.

56. The compound or salt of claim 55, wherein R⁷ is C₁alkylene-CONH—C₂alkyl.

57. The compound or salt of any one of claims 1 to 44, wherein R⁷ is C₀₋₆alkylene-C₆₋₁₀aryl or C₁₋₆alkylene-5-8 membered heteroaryl, and the aryl or heteroaryl is optionally substituted with 1-3 substituents.

58. The compound or salt of claim 57, wherein R⁷ is C₀₋₆alkylene-C₆₋₁₀aryl.

59. The compound or salt of claim 58, wherein R⁷ is C₁alkylene-C₆aryl.

60. The compound or salt of claim 58, wherein R⁷ is C₂alkylene-C₆aryl.

61. The compound or salt of any one of claims 57 to 60, wherein C₆₋₁₀aryl is substituted with 1 substituent selected from C₁₋₆alkoxy and halo.

62. The compound or salt of claim 46, wherein C₆₋₁₀aryl is substituted with chloro.

63. The compound or salt of claim 57, wherein R⁷ is C₂alkylene-pyridyl, optionally substituted with 1-3 substituents.

64. The compound or salt of any one of claims 1 to 62, wherein R⁸ is C₁₋₆alkyl.

65. The compound or salt of claim 64, wherein R⁸ is methyl.

66. The compound or salt of any one of claims 1 to 41, wherein R⁷ and R⁸ together with the nitrogen to which they are attached form a 5-12 membered heterocycle.

67. The compound or salt of claim 66, wherein R⁷ and R⁸ together with the nitrogen to which they are attached form

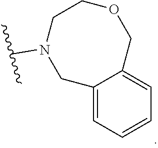 [SMILES: *N1CCOCc2ccccc2C1]

68. A compound having a structure of Formula (II), or a pharmaceutically acceptable salt thereof: 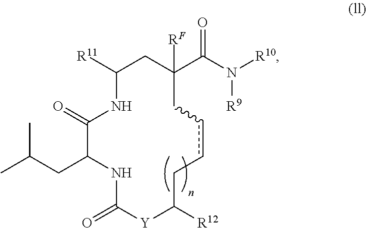
wherein
Y is O or a bond;
R^{F} is H, or
R^{F} and R¹⁰ together with the atoms to which they are attached form a five-membered heterocycle;
R⁹ is C₁₋₆alkyl, or C₁₋₆alkylene-C₆₋₁₀aryl, wherein C₆₋₁₀aryl is optionally substituted with 1-3 substituents independently selected from C₁₋₃alkoxy and halo;
R¹⁰ is H or C₁₋₆alkyl;
R¹¹ is C₁₋₆alkylene-OH, C₁₋₆alkylene-OH substituted with PO(OCH₂CH₂)₂, CHO, or (CO)₁₋₂NR¹³R¹⁴; or
R¹⁰ and R¹¹ together with the atoms to which they are attached form a six-membered heterocycle optionally substituted with 1-3 substituents independently selected from OH and CONR¹³R¹⁴;
R¹² is H, C₁₋₆alkyl, or C₁₋₆alkylene-C₆₋₁₀aryl, wherein C₆₋₁₀aryl is optionally substituted with 1-3 substituents independently selected from C₁₋₃alkoxy and halo;
R¹³ and R¹⁴ are each independently H, C₁₋₆alkyl, or C₁₋₆alkoxy; [symbol]
indicates a single or a double bond; and
n is 1-3.

69. The compound or salt of claim 68, wherein Y is O.

70. The compound or salt of claim 68, wherein Y is a bond.

71. The compound or salt of any one of claims 68 to 70, wherein n is 1.

72. The compound or salt of any one of claims 68 to 70, wherein n is 2.

73. The compound or salt of any one of claims 68 to 70, wherein n is 3.

74. The compound or salt of any one of claims 68 to 73, wherein R^{F} is H.

75. The compound or salt of any one of claims 68 to 73, wherein R^{F} and R¹⁰ together with the atoms to which they are attached form a five-membered heterocycle.

76. The compound or salt of any one of claims 68 to 75, having a structure of Formula (IIA), (IIB), (IIC), or (IID):

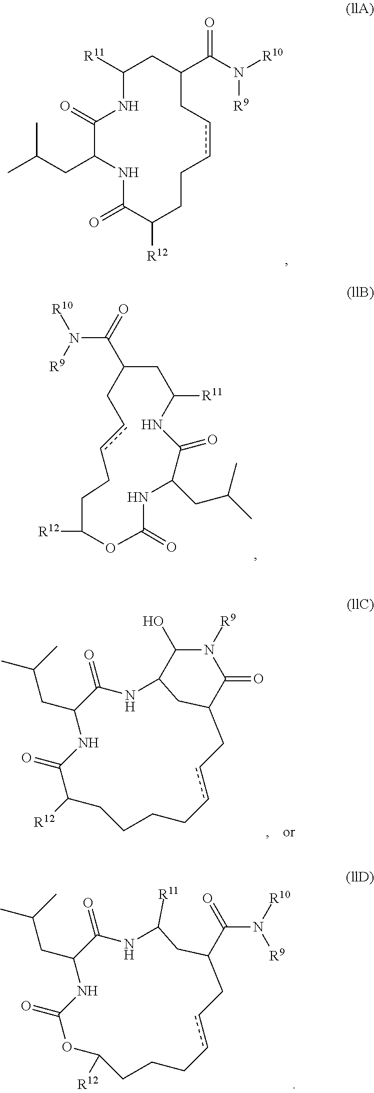

77. The compound or salt of any one R⁹ is methyl

78. R¹⁰ is methyl.

79. The compound or salt of any one of claims 68 to 74, wherein R¹⁰ and R¹¹ together with the atoms to which they are attached form a six-membered heterocycle.

80. The compound or salt of claim 79, wherein six-membered heterocycle is substituted with 1-3 substituents independently selected from OH and CONR¹³R¹⁴.

81. The compound or salt of claim 79, wherein six-membered heterocycle is substituted with OH.

82. The compound or salt of any one of claims 68 to 77, wherein R¹¹ is C₁₋₆alkylene-OH substituted with PO(OCH₂CH₂)₂.

83. The compound or salt of any one of claims 68 to 77, wherein R¹¹ is CHO.

84. The compound or salt of any one of claims 68 to 77, wherein R¹¹ is CONR¹³R¹⁴.

85. The compound or salt of claim 84, wherein R¹³ is C₁₋₆alkyl.

86. The compound or salt of claim 85, wherein R¹³ is methyl.

87. The compound or salt of any one of claims 84 to 86, wherein R¹⁴ is C₁₋₆alkoxy.

88. The compound or salt of claim 87, wherein R¹⁴ is methoxy.

89. The compound or salt of any one of claims 68 to 88, wherein R¹² is C₁₋₆alkyl.

90. The compound or salt of claim 71, wherein R¹² is C₇alkyl.

91. The compound or salt of any one of claims 68 to 88, wherein R¹² is C₁₋₆alkylene-C₆₋₁₀aryl.

92. The compound or salt of any one of claims 68 to 91, wherein [symbol] indicates a single bond.

93. The compound or salt of any one of claims 68 to 91, wherein [symbol] indicates a double bond.

94. The compound or salt of claim 93, wherein the double bond is cis.

95. The compound or salt of claim 93, wherein the double bond is trans.

96. A compound as recited in Table A, or a pharmaceutically acceptable salt thereof.

97. A compound as recited in Table B, or a pharmaceutically acceptable salt thereof.

98. A compound selected from A6, A8, A10, A15, A18, A27, A57, B22, or B37, or a pharmaceutically acceptable salt thereof.

99. A pharmaceutical formulation comprising the compound or salt of any one of claims 1 to 98 and a pharmaceutically acceptable excipient.

100. A method for treating or preventing a viral infection in a host, comprising administering to the host a therapeutic amount of the compound or salt of any one of claims 1 to 98.

101. The method of claim 100, wherein the viral infection is coronavirus infection, calicivirus infection, or picornavirus infection.

102. The method of claim 101, wherein the viral infection is a calicivirus infection.

103. The method of claim 102, wherein the calicivirus infection is a norovirus infection.

104. The method of claim 103, wherein the viral infection is a coronavirus infection.

105. The method of claim 104, wherein the coronavirus infection is severe acute respiratory syndrome (SARS), Middle East respiratory syndrome (MERS), or Coronavirus disease 2019 (COVID-19).

106. The method of claim 103, wherein the viral infection is a picornavirus infection.

107. The method of claim 102, wherein the picornavirus infection is rhinovirus infection.

108. The method of claim 107 wherein the rhinovirus infection is a rhinovirus A, rhinovirus B, or rhinovirus C infection.

109. The method of claim 101, wherein the coronavirus is an alpha coronavirus.

110. The method of claim 101, wherein the coronavirus is a beta coronavirus.
